(19) United States
(12) Patent Application Publication
(10) Pub. No.: US 2024/0018133 A1
(43) Pub. Date: Jan. 18, 2024

(54) MODULATORS OF THE INTEGRATED STRESS PATHWAY

(71) Applicants: Calico Life Sciences LLC, South San Francisco, CA (US); AbbVie Inc., North Chicago, IL (US)

(72) Inventors: Kathleen Ann Martin, San Francisco, CA (US); Carmela Sidrauski, Saratoga, CA (US); Jennifer M. Frost, Gurnee, IL (US); Yunsong Tong, Libertyville, IL (US); Xiangdong Xu, Vernon Hills, IL (US); SeungWon Chung, Libertyville, IL (US); Qingwei Zhang, Libertyville, IL (US); Lei Shi, Vernon Hills, IL (US); Kathleen J. Murauski, Libertyville, IL (US); Michael J. Dart, Highland Park, IL (US); John T. Randolph, Libertyville, IL (US); Hanae Benelkebir, Nottingham (GB)

(21) Appl. No.: 18/462,918

(22) Filed: Sep. 7, 2023

Related U.S. Application Data
(63) Continuation of application No. 16/863,737, filed on Apr. 30, 2020, now abandoned.
(60) Provisional application No. 62/840,945, filed on Apr. 30, 2019.

(51) Int. Cl. C07D 413/12; C07D 209/08; C07D 265/36; C07D 307/83; C07D 307/85; C07D 311/24; C07D 317/68; C07D 319/20; C07D 405/12; C07D 405/14; C07D 417/12; C07D 493/04; C07D 493/08

(52) U.S. Cl. CPC C07D 413/12; C07D 209/08; C07D 265/36; C07D 307/83; C07D 307/85; C07D 311/24; C07D 317/68; C07D 319/20; C07D 405/12; C07D 405/14; C07D 417/12; C07D 493/04; C07D 493/08; A61K 45/06

(57) ABSTRACT

Provided herein are compounds, compositions, and methods useful for modulating the integrated stress response (ISR) and for treating related diseases, disorders and conditions.

### CROSS REFERENCE TO RELATED APPLICATIONS

This application is a continuation of U.S. application Ser. No. 16/863,737, filed on Apr. 30, 2020, which claims the benefit of, and priority to, U.S. Provisional Application No. 62/840,945, filed on Apr. 30, 2019, the contents of each of which are hereby incorporated by reference in their entireties.

### BACKGROUND

In metazoa, diverse stress signals converge at a single phosphorylation event at serine 51 of a common effector, the translation initiation factor eIF2α. This step is carried out by four eIF2α kinases in mammalian cells: PERK, which responds to an accumulation of unfolded proteins in the endoplasmic reticulum (ER), GCN2 to amino acid starvation and UV light, PKR to viral infection and metabolic stress, and HRI to heme deficiency. This collection of signaling pathways has been termed the “integrated stress response” (ISR), as they converge on the same molecular event. eIF2α phosphorylation results in an attenuation of translation with consequences that allow cells to cope with the varied stresses (Wek, R. C. et al, Biochem Soc Trans (2006) 34(Pt 1):7-11).

eIF2 (which is comprised of three subunits, α, β and γ) binds GTP and the initiator Met-tRNA to form the ternary complex (eIF2-GTP-Met-tRNAᵢ), which, in turn, associates with the 40S ribosomal subunit scanning the 5′UTR of mRNAs to select the initiating AUG codon. Upon phosphorylation of its α-subunit, eIF2 becomes a competitive inhibitor of its GTP-exchange factor (GEF), eIF2B (Hinnebusch, A. G. and Lorsch, J. R. Cold Spring Harbor Perspect Biol (2012) 4(10)). The tight and nonproductive binding of phosphorylated eIF2 to eIF2B prevents loading of the eIF2 complex with GTP, thus blocking ternary complex formation and reducing translation initiation (Krishnamoorthy, T. et al, Mol Cell Biol (2001) 21(15):5018-5030). Because eIF2B is less abundant than eIF2, phosphorylation of only a small fraction of the total eIF2 has a dramatic impact on eIF2B activity in cells.

eIF2B is a complex molecular machine, composed of five different subunits, eIF2B1 through eIF2B5. eIF2B5 catalyzes the GDP/GTP exchange reaction and, together with a partially homologous subunit eIF2B3, constitutes the “catalytic core” (Williams, D. D. et al, J Biol Chem (2001) 276:24697-24703). The three remaining subunits (eIF2B1, eIF2B2, and eIF2B4) are also highly homologous to one another and form a “regulatory sub-complex” that provides binding sites for eIF2B's substrate eIF2 (Dev, K. et al, Mol Cell Biol (2010) 30:5218-5233). The exchange of GDP with GTP in eIF2 is catalyzed by its dedicated guanine nucleotide exchange factor (GEF) eIF2B. eIF2B exists as a decamer (B1₂B2₂ B3₂ B4₂ B5₂) or dimer of two pentamers in cells (Gordiyenko, Y. et al, Nat Commun (2014) 5:3902; Wortham, N.C. et al, FASEB J (2014) 28:2225-2237). Molecules such as ISRIB interact with and stabilize the eIF2B dimer conformation, thereby enhancing intrinsic GEF activity and making cells less sensitive to the cellular effects of phosphorylation of eIF2α (Sidrauski, C. et al, eLife (2015) e07314; Sekine, Y. et al, Science (2015) 348:1027-1030). As such, small molecule therapeutics that can modulate eIF2B activity may have the potential to attenuate the PERK branch of the UPR and the overall ISR, and therefore may be used in the prevention and/or treatment of various diseases, such as a neurodegenerative disease, a leukodystrophy, cancer, an inflammatory disease, a musculoskeletal disease, or a metabolic disease.

### SUMMARY OF THE INVENTION

The present disclosure is directed, at least in part, to compounds, compositions, and methods for the modulation of eIF2B (e.g., activation of eIF2B) and the attenuation of the ISR signaling pathway. In some embodiments, disclosed herein is an eIF2B modulator (e.g., an eIF2B activator) comprising a compound of Formula (I) or a pharmaceutically acceptable salt, solvate, hydrate, tautomer, N-oxide, or stereoisomer thereof. In other embodiments, disclosed herein are methods of using a compound of Formula (I) or a pharmaceutically acceptable salt, solvate, hydrate, tautomer, N-oxide, or stereoisomer thereof for the treatment of a disease or disorder, e.g., a neurodegenerative disease, a leukodystrophy, cancer, an inflammatory disease, a musculoskeletal disease, a metabolic disease, or a disease or disorder associated with impaired function of eIF2B or components in the ISR pathway (e.g., eIF2 pathway).

For example, disclosed herein is a compound of Formula (I):

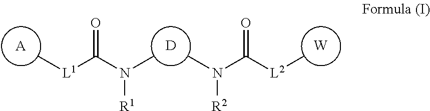

or a pharmaceutically acceptable salt, solvate, hydrate, tautomer, N-oxide, or stereoisomer thereof, wherein:
D is a bridged bicyclic cycloalkyl, bridged bicyclic heterocyclyl, or cubanyl, wherein each bridged bicyclic cycloalkyl, bridged bicyclic heterocyclyl, or cubanyl is optionally substituted on one or more available carbons with 1-4 R^{X}; and wherein if the bridged bicyclic heterocyclyl contains a substitutable nitrogen moiety, the substitutable nitrogen may be optionally substituted by R^{N1};
L¹ is a bond, C₁-C₆ alkylene, 2-7 membered heteroalkylene, —NR^{N2}—, or —O—, wherein C₁-C₆ alkylene or 2-7 membered heteroalkylene is optionally substituted with 1-5 R^{L1};
L² is a bond, C₁-C₆ alkylene, or 2-7 membered heteroalkylene, wherein C₁-C₆ alkylene or 2-7 membered heteroalkylene is optionally substituted with 1-5 R^{L2};
R¹ is hydrogen or C₁-C₆ alkyl;
R² is hydrogen or C₁-C₆ alkyl;
W is a 8-10 membered, partially unsaturated, fused bicyclic ring moiety comprising a 5-6 membered heterocyclyl fused to a phenyl or 5-6-membered heteroaryl; wherein the heterocyclyl may be optionally substituted on one or more available carbons with 1-4 R^{W1}; and wherein the phenyl or heteroaryl may optionally be substituted on one or more available unsaturated carbons with 1-4 R^{W2}; and wherein if the heterocyclyl contains a substitutable nitrogen moiety, the substitutable nitrogen may optionally be substituted with R^{N3};
A is phenyl or 5-6-membered heteroaryl, wherein phenyl or 5-6-membered heteroaryl is optionally substituted on one or more available carbons with 1-5 R^{Y}; and wherein if the 5-6-membered heteroaryl contains a substitutable nitrogen moiety, the substitutable nitrogen may be optionally substituted by R^{N4};
each R^{L1} is independently selected from the group consisting of hydrogen, C₁-C₆ alkyl, hydroxy-C₁-C₆ alkyl, halo-C₁-C₆ alkyl, amino-C₁-C₆ alkyl, cyano-C₁-C₆ alkyl, oxo, halo, cyano, —OR^{A}, —NR^{B}R^{C}, —NR^{B}C(O)R^{D}, —C(O)NR^{B}R^{C}, —C(O)R^{D}, —C(O)OH, —C(O)OR^{D}, —SR^{E}, —S(O)R^{D}, and —S(O)₂R^{D};
each R^{L2} is independently selected from the group consisting of hydrogen, C₁-C₆ alkyl, hydroxy-C₁-C₆ alkyl, halo-C₁-C₆ alkyl, amino-C₁-C₆ alkyl, cyano-C₁-C₆ alkyl, oxo, halo, cyano, —OR^{A}, —NR^{B}R^{C}, —N^{B}C(O)R^{D}, —C(O)NR^{B}R^{C}, —C(O)R^{D}, —C(O)OH, —C(O)OR^{D}, —SR^{E}, —S(O)R^{D}, and —S(O)₂R^{D};
R^{N1} is selected from the group consisting of hydrogen, C₁-C₆ alkyl, hydroxy-C₂-C₆ alkyl, halo-C₂-C₆ alkyl, amino-C₂-C₆ alkyl, cyano-C₂-C₆ alkyl, —C(O)NR^{B}R^{C}, —C(O)R^{D}, —C(O)OR^{D}, and —S(O)₂R^{D};
R^{N2} is selected from the group consisting of hydrogen, C₁-C₆ alkyl, hydroxy-C₂-C₆ alkyl, halo-C₂-C₆ alkyl, amino-C₂-C₆ alkyl, cyano-C₂-C₆ alkyl, —C(O)NR^{B}R^{C}, —C(O)R^{D}, —C(O)OR^{D}, and —S(O)₂R^{D};
R^{N3} is selected from the group consisting of hydrogen, C₁-C₆ alkyl, hydroxy-C₂-C₆ alkyl, C₁-C₆ alkyl-C₁-C₆ cycloalkyl, C₁-C₆ alkenyl, —C(O)—C₁-C₆ alkyl, —C(O)—C₁-C₆ cycloalkyl, C₁-C₆ alkyl-CO₂H, C₁-C₆ alkyl-CO₂—C₁-C₆ alkyl, —C(O)—C₁-C₃ alkyl-O—C₁-C₃ alkyl-O—C₁-C₃ alkyl, —C(O)-phenyl, —C(O)-heteroaryl, —C(O)-heterocyclyl, —S—C₁-C₆ alkyl, —S(O)₂—C₁-C₆ alkyl, —S(O)₂-phenyl, —S(O)₂-heteroaryl, —C(O)NR^{B}R^{C} and —C(O)OR^{D};
   wherein C₁-C₆ alkyl, hydroxy-C₂-C₆ alkyl, C₁-C₆ alkyl-C₁-C₆ cycloalkyl, C₁-C₆ alkenyl, C(O)—C₁-C₆ alkyl, —C(O)—C₁-C₆ cycloalkyl, C₁-C₆ alkyl-CO₂H, C₁-C₆ alkyl-CO₂—C₁-C₆ alkyl, —C(O)-heterocyclyl, —S—C₁-C₆ alkyl and —S(O)₂—C₁-C₆ alkyl may optionally be substituted by one or more substituents each independently selected from the group consisting of fluoro, hydroxyl, C₁-C₆ alkoxy, C₁-C₆ alkyl (optionally substituted by one, two or three fluorine atoms) and S(O)_{w}C₁₋₆ alkyl (wherein w is 0, 1 or 2); and
   wherein —C(O)-phenyl, —C(O)-heteroaryl, —S(O)₂-phenyl and —S(O)₂-heteroaryl may optionally be substituted by one or more substituents each independently selected from the group consisting of halogen, hydroxyl, C₁-C₆ alkyl (optionally substituted by one, two or three fluorine atoms), C₁-C₆ alkoxy (optionally substituted by one, two or three fluorine atoms), S(O₂)NR^{B}R^{C} and SO₂F;
R^{N4} is selected from the group consisting of hydrogen, C₁-C₆ alkyl, hydroxy-C₂-C₆ alkyl, halo-C₂-C₆ alkyl, amino-C₂-C₆ alkyl, cyano-C₂-C₆ alkyl, C₃-C₆ cycloalkyl, phenyl, 5-6-membered heteroaryl, —C(O)NR^{B}R^{C}, —C(O)R^{D}, —C(O)OR^{D}, and —S(O)₂R^{D};
   wherein C₃-C₆ cycloalkyl, phenyl, and 5-6-membered heteroaryl may optionally be substituted by one or more substituents each independently selected from the group consisting of halo, C₁-C₆ alkyl (optionally substituted by one, two or three fluorine atoms), and C₁-C₆ alkoxy (optionally substituted by one, two or three fluorine atoms).
each R^{W1} is independently selected from the group consisting of hydrogen, C₁-C₆ alkyl (optionally substituted by —CO₂H), hydroxy-C₁-C₆ alkyl, hydroxy-C₂-C₆ alkyl-O—, halo-C₁-C₆ alkyl, amino-C₁-C₆ alkyl, cyano-C₁-C₆ alkyl, oxo, C═NOH, halo, cyano, —OR^{A}, —NR^{B}R^{C}, —NR^{B}R^{CC}, —R^{B}C(O)R^{D}, —C(O)NR^{B}R^{C}, —C(O)R^{D}, —C(O)OH, —C(O)OR^{D}, —SR^{E}, —S(O)R^{D}, and —S(O)₂R^{D};
each R^{W2} is independently selected from the group consisting of hydrogen, C₁-C₆ alkyl, hydroxy-C₁-C₆ alkyl, hydroxy-C₂-C₆ alkyl-O—, halo-C₁-C₆ alkyl, halo-C₁-C₆ alkoxy, amino-C₁-C₆ alkyl, cyano-C₁-C₆ alkyl, halo, cyano, —OR^{A}, —NR^{B}R^{C}, —N^{B}C(O)R^{D}, —C(O)NR^{B}R^{C}, —C(O)R^{D}, —C(O)OH, —C(O)OR^{D}, —S(R^{F})ₘ, —S(O)R^{D}, and —S(O)₂R^{D}; or
2 R^{W2} groups on adjacent atoms, together with the atoms to which they are attached, form a 3-7-membered fused cycloalkyl, 3-7-membered fused heterocyclyl, fused aryl, or 5-6 membered fused heteroaryl, each of which is optionally substituted with 1-5 R^{X};
each R^{X} is independently selected from the group consisting of hydrogen, C₁-C₆ alkyl, hydroxy-C₁-C₆ alkyl, halo-C₁-C₆ alkyl, amino-C₁-C₆ alkyl, cyano-C₁-C₆ alkyl, oxo, halo, cyano, —OR^{A}, —NR^{B}R^{C}, —N^{B}C(O)R^{D}, —C(O)NR^{B}R^{C}, —C(O)R^{D}, —C(O)OH, —C(O)OR^{D}, —SR^{E}, —S(O)R^{D}, and —S(O)₂R^{D};
each R^{Y} is independently selected from the group consisting of hydrogen, C₁-C₆ alkyl, hydroxy-C₁-C₆ alkyl, halo-C₁-C₆ alkyl, halo-C₁-C₆ alkoxy, amino-C₁-C₆ alkyl, cyano-C₁-C₆ alkyl, C₃-C₆ cycloalkyl, 3-7 membered heterocyclyl, halo-C₁-C₆ alkyl-3-7 membered heterocyclyl, halo, cyano, —OR^{A}, —NR^{B}R^{C}, —N^{B}C(O)R^{D}, —C(O)NR^{B}R^{C}, —C(O)R^{D}, —C(O)OH, —C(O)OR^{D}, —S(R^{F})ₘ, —S(O)R^{D}, —S(O)₂R^{D}, and G¹; or
2 R^{Y} groups on adjacent atoms, together with the atoms to which they are attached form a 3-7-membered fused cycloalkyl, 3-7-membered fused heterocyclyl, fused aryl, or 5-6 membered fused heteroaryl, each of which is optionally substituted with 1-5 R^{X};
each G¹ is independently 3-7-membered cycloalkyl, 3-7-membered heterocyclyl, aryl, or 5-6-membered heteroaryl, wherein each 3-7-membered cycloalkyl, 3-7-membered heterocyclyl, aryl, or 5-6-membered heteroaryl is optionally substituted with 1-3 R^{Z};
each R^{Z} is independently selected from the group consisting of C₁-C₆ alkyl, hydroxy-C₁-C₆ alkyl, halo-C₁-C₆ alkyl, halo, cyano, —OR^{A}, —NR^{B}R^{C}, —N^{B}C(O)R^{D}, —C(O)NR^{B}R^{C}, —C(O)R^{D}, —C(O)OH, —C(O)OR^{D}, and —S(O)₂R^{D};
R^{A} is, at each occurrence, independently hydrogen, C₁-C₆ alkyl, halo-C₁-C₆ alkyl, —C(O)NR^{B}R^{C}, —C(O)R^{D}, or —C(O)OR^{D};
each of R^{B} and R^{C} is independently hydrogen or C₁-C₆ alkyl;
R^{B} and R^{C} together with the atom to which they are attached form a 3-7-membered heterocyclyl ring optionally substituted with 1-3 R^{Z};
each R^{CC} is independently selected from the group consisting of hydroxy-C₁-C₆ alkyl, halo-C₁-C₆ alkyl, C₁-C₆ alkyl-CO₂H, C₁-C₆ alkyl-CO₂—C₁-C₆ alkyl, C(O) C₁-C₆ alkyl, S(O)₂—C₁-C₆ alkyl, 3-6-membered cycloalkyl and 4-6-membered heterocyclyl; wherein 3-6-membered cycloalkyl and 4-6-membered heterocyclyl may optionally be substituted by one or more substituents each independently selected from the group consisting of C₁-C₆ alkyl, hydroxy-C₁-C₆ alkyl, halo-C₁-C₆ alkyl, hydroxyl, halo and —C(O)OH;
each R^{D} is independently C₁-C₆ alkyl or halo-C₁-C₆ alkyl;
each R^{E} is independently hydrogen, C₁-C₆ alkyl, or halo-C₁-C₆ alkyl;
each R^{F} is independently hydrogen, C₁-C₆ alkyl, or halo; and
m is 1 when RF is hydrogen or C₁-C₆ alkyl, or 5 when R^{F} is halo.

In some embodiments, the compound of Formula (I) is a compound of Formula (I-a):

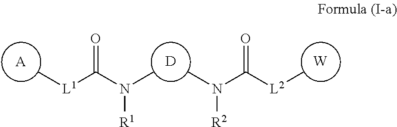

or a pharmaceutically acceptable salt, solvate, hydrate, tautomer, N-oxide, or stereoisomer thereof, wherein:
D is bicyclo[1.1.1]pentanyl or bicyclo[2.2.2]octanyl, each of which is optionally substituted with 1-4 R^{X} groups;
L¹ is selected from the group consisting of a bond and CH₂O—*, wherein “-*” indicates the attachment point to A;
L² is a bond;
R¹ is selected from the group consisting of hydrogen and CH₃;
R² is selected from the group consisting of hydrogen and CH₃;
A is phenyl, pyrazinyl or pyridyl, each of which is optionally substituted with 1-5 R^{Y}, groups;
W is a benzo[d][1,3]dioxole, 3,4-dihydro-2H-benzo[b][1,4]oxazine, chromane, chromene, chroman-4-one, chroman-4-ol, chroman-4-one oxime, 2H-benzo[b][1,4]oxazin-3(4H)-one, 2,3-dihydrobenzo[b][1,4]dioxine, indoline, 2,3-dihydrobenzofuran, benzofuran-3(2H)-one, 4H-chromen-4-ol or 4H-chromen-4-one moiety; wherein each of which is attached to L² through a carbon atom, and wherein each of which is optionally substituted on one or more available aromatic carbon atoms with 1-4 R^{W2} groups; and wherein 3,4-dihydro-2H-benzo[b][1,4]oxazine, 2H-benzo[b][1,4]oxazin-3(4H)-one, and indoline may be optionally substituted on an available nitrogen atom with hydrogen or CH₃;
each R^{W2} is independently selected from the group consisting of hydrogen, chloro, fluoro, CHF₂, CF₃, CH₃, CH₂CH₃, CH(CH₃)₂, OCH₃, OCHF₂, OCF₃, OCH₂CF₃, OCH(CH₃)₂, and CN; or
2 R^{W2} groups on adjacent carbons, together with the atoms to which they are attached form a 1,3-dioxolanyl ring, which is optionally substituted with 1-2 fluorine atoms;
each R^{X} is independently fluoro, oxo, OH, OCH₃, C(O)OH, or C(O)OCH₃; and
each R^{Y} is independently chloro, fluoro, CHF₂, CF₃, CH₃, CH₂CH₃, CH(CH₃)₂, OCH₃, OCHF₂, OCF₃, OCH₂CF₃, OCH(CH₃)₂, or CN; or
2 R^{Y} groups on adjacent atoms, together with the atoms to which they are attached form a 1,3-dioxolanyl ring, which is optionally substituted with 1-2 fluorine atoms.

In some embodiments, the compound of Formula (I) is a compound of Formula (I-b):

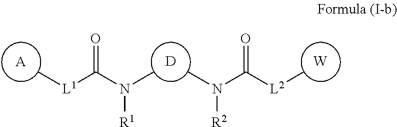

or a pharmaceutically acceptable salt, solvate, hydrate, tautomer, N-oxide, or stereoisomer thereof, wherein:
D is bicyclo[1.1.1]pentanyl or bicyclo[2.2.2]octanyl, each of which is optionally substituted with 1-4 R^{X} groups;
L¹ is selected from the group consisting of a bond and CH₂O—*, wherein “-*” indicates the attachment point to A;
L² is CH₂—*, wherein “-*” indicates the attachment point to W;
R¹ is selected from the group consisting of hydrogen and CH₃;
R² is selected from the group consisting of hydrogen and CH₃;
A is phenyl, pyrazinyl or pyridyl, each of which is optionally substituted with 1-5 R^{Y} groups;
W is an indoline or tetrahydroisoquinoline moiety; wherein indoline or tetrahydroisoquinoline is attached to L² through a nitrogen atom, and wherein indoline or tetrahydroisoquinoline is optionally substituted on one or more available unsaturated carbon atoms with 1-4 R^{W2} groups;
each R^{W2} is independently selected from the group consisting of hydrogen, chloro, fluoro, CHF₂, CF₃, CH₃, CH₂CH₃, CH(CH₃)₂, OCH₃, OCHF₂, OCF₃, OCH₂CF₃, OCH(CH₃)₂, and CN; or
2 R^{W2} groups on adjacent carbons, together with the atoms to which they are attached form a 1,3-dioxolanyl ring, which is optionally substituted with 1-2 fluorine atoms;
each R^{X} is independently fluoro, oxo, OH, OCH₃, C(O)OH, or C(O)OCH₃; and
each R^{Y} is independently chloro, fluoro, CHF₂, CF₃, CH₃, CH₂CH₃, CH(CH₃)₂, OCH₃, OCHF₂, OCF₃, OCH₂CF₃, OCH(CH₃)₂, or CN; or
2 R^{Y} groups on adjacent atoms, together with the atoms to which they are attached form a 1,3-dioxolanyl ring, which is optionally substituted with 1-2 fluorine atoms.

In some embodiments, a compound disclosed herein, or a pharmaceutically acceptable salt thereof is formulated as a pharmaceutically acceptable composition comprising a disclosed compound and a pharmaceutically acceptable carrier.

In some embodiments, a compound disclosed herein is selected from a compound set forth in Table 1 or a pharmaceutically acceptable salt, solvate, hydrate, tautomer, N-oxide or stereoisomer thereof.

In another aspect, the present invention features a method of treating a neurodegenerative disease, a leukodystrophy, a cancer, an inflammatory disease, an autoimmune disease, a viral infection, a skin disease, a fibrotic disease, a hemoglobin disease, a kidney disease, a hearing loss condition, an ocular disease, a musculoskeletal disease, a metabolic disease, or a mitochondrial disease, or a disease or disorder associated with impaired function of eIF2B or components in the ISR pathway (e.g., eIF2 pathway) in a subject, wherein the method comprises administering a compound of Formula (I) or a pharmaceutically acceptable salt, solvate, hydrate, tautomer, N-oxide or stereoisomer thereof, or a composition thereof, to a subject.

In some embodiments, the method comprises the treatment of a neurodegenerative disease. In some embodiments, the neurodegenerative disease comprises a leukodystrophy, a leukoencephalopathy, a hypomyelinating or demyelinating disease, an intellectual disability syndrome, a cognitive impairment, a glial cell dysfunction, or a brain injury. In some embodiments, the neurodegenerative disease comprises vanishing white matter disease, childhood ataxia with CNS hypo myelination, Alzheimer's disease, amyotrophic lateral sclerosis, Creutzfeldt-Jakob disease, frontotemporal dementia, Gerstmann-Straussler-Scheinker disease, Huntington's disease, dementia, kuru, multiple sclerosis, Parkinson's disease, or a prion disease. In some embodiments, the neurodegenerative disease comprises vanishing white matter disease.

In some embodiments, the method comprises the treatment of cancer. In some embodiments, the cancer comprises pancreatic cancer, breast cancer, multiple myeloma, or a cancer of the secretory cells.

In some embodiments, the method comprises the treatment of an inflammatory disease. In some embodiments, the inflammatory disease comprises postoperative cognitive dysfunction, arthritis, systemic lupus erythematosus (SLE), myasthenia gravis, diabetes), Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, Behcet's disease, Crohn's disease, ulcerative colitis, bullous pemphigoid, sarcoidosis, ichthyosis, Graves' ophthalmopathy, inflammatory bowel disease, Addison's disease, vitiligo, acne vulgaris, celiac disease, chronic prostatitis, pelvic inflammatory disease, reperfusion injury, sarcoidosis, transplant rejection, interstitial cystitis, atherosclerosis, or atopic dermatitis.

In some embodiments, the method comprises the treatment of a musculoskeletal disease. In some embodiments, the musculoskeletal disease comprises muscular dystrophy, multiple sclerosis, amyotropic lateral sclerosis, primary lateral sclerosis, progressive muscular atrophy, progressive bulbar palsy, pseudobulbar palsy, spinal muscular atrophy, progressive spinobulbar muscular atrophy, spinal cord spasticity, spinal muscle atrophy, myasthenia gravis, neuralgia, fibromyalgia, Machado-Joseph disease, cramp fasciculation syndrome, Freidrich's ataxia, a muscle wasting disorder), an inclusion body myopathy, motor neuron disease, or paralysis.

In some embodiments, the method comprises the treatment of a metabolic disease. In some embodiments, the metabolic disease comprises non-alcoholic steatohepatitis (NASH), non-alcoholic fatty liver disease (NAFLD), liver fibrosis, obesity, heart disease, atherosclerosis, arthritis, cystinosis, diabetes, phenylketonuria, proliferative retinopathy, or Kearns-Sayre disease.

In some embodiments, the method comprises the treatment of a mitochondrial disease. In some embodiments, the mitochondrial disease is associated with, or is a result of, or is caused by mitochondrial dysfunction, one or more mitochondrial protein mutations, or one or more mitochondrial DNA mutations. In some embodiments, the mitochondrial disease is a mitochondrial myopathy. In some embodiments, the mitochondrial disease is selected from the group consisting of Barth syndrome, chronic progressive external ophthalmoplegia (cPEO), Kearns-Sayre syndrome (KSS), Leigh syndrome (e.g., MILS, or maternally inherited Leigh syndrome), mitochondrial DNA depletion syndromes (MDDS, e.g., Alpers syndrome), mitochondrial encephalomyopathy (e.g., mitochondrial encephalomyopathy, lactic acidosis, and stroke-like episodes (MELAS)), mitochondrial neurogastrointestinal encephalomyopathy (MNGIE), myoclonus epilepsy with ragged red fibers (MERRF), neuropathy, ataxia, retinitis pigmentosa (NARP), Leber's hereditary optic neuropathy (LHON and Pearson syndrome.

In another aspect, the present invention features a method of treating a disease or disorder related to modulation (e.g., a decrease) in eIF2B activity or level, modulation (e.g., a decrease) of eIF2α activity or level, modulation (e.g., an increase) in eIF2α phosphorylation, modulation (e.g., an increase) of phosphorylated eIF2α pathway activity, or modulation (e.g., an increase) of ISR activity in a subject, wherein the method comprises administering a compound of Formula (I) or a pharmaceutically acceptable salt, solvate, hydrate, tautomer, N-oxide or stereoisomer thereof, or a composition thereof, to a subject. In some embodiments, the disease may be caused by a mutation to a gene or protein sequence related to a member of the eIF2 pathway (e.g., the eIF2α signaling pathway or ISR pathway).

In another aspect, the present invention features a method of treating cancer in a subject, the method comprising administering to the subject a compound of Formula (I) in combination with an immunotherapeutic agent.

### DETAILED DESCRIPTION

The present invention features compounds, compositions, and methods comprising a compound of Formula (I) or a pharmaceutically acceptable salt, solvate, hydrate, tautomer, N-oxide or stereoisomer thereof for use, e.g., in the modulation (e.g., activation) of eIF2B and the attenuation of the ISR signaling pathway.

### Definitions

### Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

The abbreviations used herein have their conventional meaning within the chemical and biological arts. The chemical structures and formulae set forth herein are constructed according to the standard rules of chemical valency known in the chemical arts.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, N Y, 1962); and Wilen, Tables of Resolving Agents and Optical Resolutions p. 268 (E. L. Eliel, Ed., Univ. of Notre Dame Press, Notre Dame, IN 1972). The invention additionally encompasses compounds described herein as individual isomers substantially free of other isomers, and alternatively, as mixtures of various isomers.

As used herein a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. The term “enantiomerically pure” or “pure enantiomer” denotes that the compound comprises more than 75% by weight, more than 80% by weight, more than 85% by weight, more than 90% by weight, more than 91% by weight, more than 92% by weight, more than 93% by weight, more than 94% by weight, more than 95% by weight, more than 96% by weight, more than 97% by weight, more than 98% by weight, more than 99% by weight, more than 99.5% by weight, or more than 99.9% by weight, of the enantiomer. In certain embodiments, the weights are based upon total weight of all enantiomers or stereoisomers of the compound.

In the compositions provided herein, an enantiomerically pure compound can be present with other active or inactive ingredients. For example, a pharmaceutical composition comprising enantiomerically pure R-compound can comprise, for example, about 90% excipient and about 10% enantiomerically pure R-compound. In certain embodiments, the enantiomerically pure R-compound in such compositions can, for example, comprise, at least about 95% by weight R-compound and at most about 5% by weight S-compound, by total weight of the compound. For example, a pharmaceutical composition comprising enantiomerically pure S-compound can comprise, for example, about 90% excipient and about 10% enantiomerically pure S-compound. In certain embodiments, the enantiomerically pure S-compound in such compositions can, for example, comprise, at least about 95% by weight S-compound and at most about 5% by weight R-compound, by total weight of the compound. In certain embodiments, the active ingredient can be formulated with little or no excipient or carrier.

Compound described herein may also comprise one or more isotopic substitutions. For example, H may be in any isotopic form, including ¹H, ²H (D or deuterium), and ³H (T or tritium); C may be in any isotopic form, including ¹²C, ¹³C, and ¹⁴C; O may be in any isotopic form, including ¹⁶O and ¹⁸O; and the like.

The articles “a” and “an” may be used herein to refer to one or to more than one (i.e. at least one) of the grammatical objects of the article. By way of example “an analogue” means one analogue or more than one analogue.

When a range of values is listed, it is intended to encompass each value and sub-range within the range. For example “C₁-C₆ alkyl” is intended to encompass, C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆ alkyl.

The following terms are intended to have the meanings presented therewith below and are useful in understanding the description and intended scope of the present invention.

“Alkyl” refers to a radical of a straight-chain or branched saturated hydrocarbon group having from 1 to 20 carbon atoms (“C₁-C₂₀ alkyl”). In some embodiments, an alkyl group has 1 to 12 carbon atoms (“C₁-C₁₂ alkyl”). In some embodiments, an alkyl group has 1 to 8 carbon atoms (“C₁-C₈ alkyl”). In some embodiments, an alkyl group has 1 to 6 carbon atoms (“C₁-C₆ alkyl”). In some embodiments, an alkyl group has 1 to 5 carbon atoms (“C₁-C₈ alkyl”). In some embodiments, an alkyl group has 1 to 4 carbon atoms (“C₁-C₄ alkyl”). In some embodiments, an alkyl group has 1 to 3 carbon atoms (“C₁-C₃ alkyl”). In some embodiments, an alkyl group has 1 to 2 carbon atoms (“C₁-C₂ alkyl”). In some embodiments, an alkyl group has 1 carbon atom (“C₁ alkyl”). In some embodiments, an alkyl group has 2 to 6 carbon atoms (“C₂-C₆ alkyl”). Examples of C₁-C₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₅) and the like. Each instance of an alkyl group may be independently optionally substituted, i.e., unsubstituted (an “unsubstituted alkyl”) or substituted (a “substituted alkyl”) with one or more substituents; e.g., for instance from 1 to 5 substituents, 1 to 3 substituents, or 1 substituent. In certain embodiments, the alkyl group is unsubstituted C₁₋₁₀ alkyl (e.g., —CH₃). In certain embodiments, the alkyl group is substituted C₁₋₆ alkyl. Common alkyl abbreviations include Me (—CH₃), Et (—CH₂CH₃), iPr (—CH(CH₃)₂), nPr (—CH₂CH₂CH₃), n-Bu (—CH₂CH₂CH₂CH₃), or i-Bu (—CH₂CH(CH₃)₂).

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred in the present invention. The term “alkenylene,” by itself or as part of another substituent, means, unless otherwise stated, a divalent radical derived from an alkene. An alkylene group may be described as, e.g., a C₁-C₆-membered alkylene, wherein the term “membered” refers to the non-hydrogen atoms within the moiety.

“Alkenyl” refers to a radical of a straight-chain or branched hydrocarbon group having from 2 to 20 carbon atoms, one or more carbon-carbon double bonds, and no triple bonds (“C₂-C₂₀ alkenyl”). In some embodiments, an alkenyl group has 2 to 10 carbon atoms (“C₂-C₁₀ alkenyl”). In some embodiments, an alkenyl group has 2 to 8 carbon atoms (“C₂-C₅ alkenyl”). In some embodiments, an alkenyl group has 2 to 6 carbon atoms (“C₂-C₆ alkenyl”). In some embodiments, an alkenyl group has 2 to 5 carbon atoms (“C₂-C₅ alkenyl”). In some embodiments, an alkenyl group has 2 to 4 carbon atoms (“C₂-C₄ alkenyl”). In some embodiments, an alkenyl group has 2 to 3 carbon atoms (“C₂-C₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂-C₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂-C₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Each instance of an alkenyl group may be independently optionally substituted, i.e., unsubstituted (an “unsubstituted alkenyl”) or substituted (a “substituted alkenyl”) with one or more substituents e.g., for instance from 1 to 5 substituents, 1 to 3 substituents, or 1 substituent. In certain embodiments, the alkenyl group is unsubstituted C₂₋₁₀ alkenyl. In certain embodiments, the alkenyl group is substituted C₂₋₆ alkenyl.

“Aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆-C₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). An aryl group may be described as, e.g., a C₆-C₁₀-membered aryl, wherein the term “membered” refers to the non-hydrogen ring atoms within the moiety. Aryl groups include, but are not limited to, phenyl, naphthyl, indenyl, and tetrahydronaphthyl. Each instance of an aryl group may be independently optionally substituted, i.e., unsubstituted (an “unsubstituted aryl”) or substituted (a “substituted aryl”) with one or more substituents. In certain embodiments, the aryl group is unsubstituted C₆-C₁₄ aryl. In certain embodiments, the aryl group is substituted C₆-C₁₄ aryl.

In certain embodiments, an aryl group is substituted with one or more of groups selected from halo, C₁-C₈ alkyl, halo-C₁-C₈ alkyl, haloxy-C₁-C₈ alkyl, cyano, hydroxy, alkoxy C₁-C₈ alkyl, and amino.

Examples of representative substituted aryls include the following

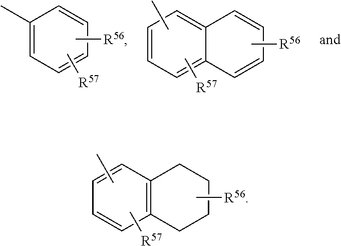

wherein one of R⁵⁶ and R⁵⁷ may be hydrogen and at least one of R⁵⁶ and R⁵⁷ is each independently selected from C₁-C₈ alkyl, halo-C₁-C₈ alkyl, 4-10 membered heterocyclyl, alkanoyl, alkoxy-C₁-C₈ alkyl, heteroaryloxy, alkylamino, arylamino, heteroarylamino, NR⁵⁸COR⁵⁹, NR⁵⁸SOR⁵⁹NR⁵⁸SO₂R⁵⁹, C(O)Oalkyl, C(O)Oaryl, CONR⁵⁸R⁵⁹, CONR⁵⁸OR⁵⁹, NR⁵⁸R⁵⁹, SO₂NR⁵⁸R⁵⁹, S-alkyl, S(O)-alkyl, S(O)₂-alkyl, S-aryl, S(O)-aryl, S(O₂)-aryl; wherein R⁵⁸ and R⁵⁹ are each independently selected from hydrogen or C₁-C₆ alkyl; or R⁵⁶ and R⁵⁷ may be joined to form a cyclic ring (saturated or unsaturated) from 5 to 8 atoms, optionally containing one or more heteroatoms selected from the group N, O, or S.

Other representative aryl groups having a fused heterocyclyl group include the following:

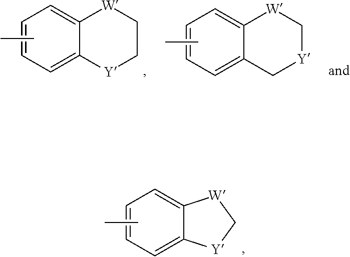

wherein each W′ is selected from C(R⁶⁶)₂, NR⁶⁶, O, and S; and each Y′ is selected from carbonyl, NR⁶⁶, O and S; and R⁶⁶ is independently hydrogen, C₁-C₈ alkyl, C₃-C₁₀ cycloalkyl, 4-10 membered heterocyclyl, C₆-C₁₀ aryl, and 5-10 membered heteroaryl.

An “arylene” and a “heteroarylene,” alone or as part of another substituent, mean a divalent radical derived from an aryl and heteroaryl, respectively. Non-limiting examples of heteroaryl groups include pyridinyl, pyrimidinyl, thiophenyl, thienyl, furanyl, indolyl, benzoxadiazolyl, benzodioxolyl, benzodioxanyl, thianaphthanyl, pyrrolopyridinyl, indazolyl, quinolinyl, quinoxalinyl, pyridopyrazinyl, quinazolinonyl, benzoisoxazolyl, imidazopyridinyl, benzofuranyl, benzothienyl, benzothiophenyl, phenyl, naphthyl, biphenyl, pyrrolyl, pyrazolyl, imidazolyl, pyrazinyl, oxazolyl, isoxazolyl, thiazolyl, furylthienyl, pyridyl, pyrimidyl, benzothiazolyl, purinyl, benzimidazolyl, isoquinolyl, thiadiazolyl, oxadiazolyl, pyrrolyl, diazolyl, triazolyl, tetrazolyl, benzothiadiazolyl, isothiazolyl, pyrazolopyrimidinyl, pyrrolopyrimidinyl, benzotriazolyl, benzoxazolyl, or quinolyl. The examples above may be substituted or unsubstituted and divalent radicals of each heteroaryl example above are non-limiting examples of heteroarylene.

“Halo” or “halogen,” independently or as part of another substituent, mean, unless otherwise stated, a fluorine (F), chlorine (Cl), bromine (Br), or iodine (I) atom. The term “halide” by itself or as part of another substituent, refers to a fluoride, chloride, bromide, or iodide atom. In certain embodiments, the halo group is either fluorine or chlorine.

Additionally, terms such as “haloalkyl” are meant to include monohaloalkyl and polyhaloalkyl. For example, the term “halo-C₁-C₆ alkyl” includes, but is not limited to, fluoromethyl, difluoromethyl, trifluoromethyl, 2,2,2-trifluoroethyl, 4-chlorobutyl, 3-bromopropyl, and the like.

The term “heteroalkyl,” by itself or in combination with another term, means, unless otherwise stated, a non-cyclic stable straight or branched chain, or combinations thereof, including at least one carbon atom and at least one heteroatom selected from the group consisting of O, N, P, Si, and S, and wherein the nitrogen and sulfur atoms may optionally be oxidized, and the nitrogen heteroatom may optionally be quaternized. The heteroatom(s) O, N, P, S, and Si may be placed at any interior position of the heteroalkyl group or at the position at which the alkyl group is attached to the remainder of the molecule. Exemplary heteroalkyl groups include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)₂, —S(O)—CH₃, —S(O)₂—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, and —O—CH₂—CH₃. Up to two or three heteroatoms may be consecutive, such as, for example, —CH₂—NH—OCH₃ and —CH₂—O—Si(CH₃)₃. Where “heteroalkyl” is recited, followed by recitations of specific heteroalkyl groups, such as —CH₂O, —NR^{B}R^{C}, or the like, it will be understood that the terms heteroalkyl and —CH₂O or —NR^{B}R^{C} are not redundant or mutually exclusive. Rather, the specific heteroalkyl groups are recited to add clarity. Thus, the term “heteroalkyl” should not be interpreted herein as excluding specific heteroalkyl groups, such as —CH₂O, —NR^{B}R^{C}, or the like.

Similarly, the term “heteroalkylene,” by itself or as part of another substituent, means, unless otherwise stated, a divalent radical derived from heteroalkyl, as exemplified, but not limited by, —CH₂O— and —CH₂CH₂O—. A heteroalkylene group may be described as, e.g., a 2-7-membered heteroalkylene, wherein the term “membered” refers to the non-hydrogen atoms within the moiety. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— may represent both —C(O)₂R′— and —R′C(O)₂—.

“Heteroaryl” refers to a radical of a 5-10 membered monocyclic or bicyclic 4n+2 aromatic ring system (e.g., having 6 or 10 π electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl). A heteroaryl group may be described as, e.g., a 6-10-membered heteroaryl, wherein the term “membered” refers to the non-hydrogen ring atoms within the moiety.

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Each instance of a heteroaryl group may be independently optionally substituted, i.e., unsubstituted (an “unsubstituted heteroaryl”) or substituted (a “substituted heteroaryl”) with one or more substituents. In certain embodiments, the heteroaryl group is unsubstituted 5-14 membered heteroaryl. In certain embodiments, the heteroaryl group is substituted 5-14 membered heteroaryl.

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl.

Examples of representative heteroaryls include the following formulae:

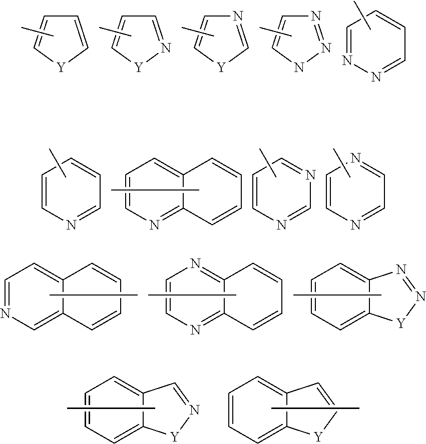

wherein each Y is selected from carbonyl, N, NR⁶⁵, O, and S; and R⁶⁵ is independently hydrogen, C₁-C₈ alkyl, C₃-C₁₀ cycloalkyl, 4-10 membered heterocyclyl, C₆-C₁₀ aryl, and 5-10 membered heteroaryl.
“Cycloalkyl” refers to a radical of a non-aromatic cyclic hydrocarbon group having from 3 to 10 ring carbon atoms (“C₃-C₁₀ cycloalkyl”) and zero heteroatoms in the non-aromatic ring system. In some embodiments, a cycloalkyl group has 3 to 8 ring carbon atoms (“C₃-C₈cycloalkyl”). In some embodiments, a cycloalkyl group has 3 to 6 ring carbon atoms (“C₃-C₆ cycloalkyl”). In some embodiments, a cycloalkyl group has 3 to 6 ring carbon atoms (“C₃-C₆ cycloalkyl”). In some embodiments, a cycloalkyl group has 5 to 10 ring carbon atoms (“C₅-C₁₀ cycloalkyl”). A cycloalkyl group may be described as, e.g., a C₄-C₇-membered cycloalkyl, wherein the term “membered” refers to the non-hydrogen ring atoms within the moiety. Exemplary C₃-C₆ cycloalkyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃-C₈ cycloalkyl groups include, without limitation, the aforementioned C₃-C₆ cycloalkyl groups as well as cycloheptyl (C₇), cycloheptenyl (C₇), cycloheptadienyl (C₇), cycloheptatrienyl (C₇), cyclooctyl (C₈), cyclooctenyl (C₈), cubanyl (C₈), bicyclo[1.1.1]pentanyl (C₅), bicyclo[2.2.2]octanyl (C₈), bicyclo[2.1.1]hexanyl (C₆), bicyclo[3.1.1]heptanyl (C₇), and the like. Exemplary C₃-C₁₀ cycloalkyl groups include, without limitation, the aforementioned C₃-C₈ cycloalkyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the cycloalkyl group is either monocyclic (“monocyclic cycloalkyl”) or contain a fused, bridged or spiro ring system such as a bicyclic system (“bicyclic cycloalkyl”) and can be saturated or can be partially unsaturated. “Cycloalkyl” also includes ring systems wherein the cycloalkyl ring, as defined above, is fused with one or more aryl groups wherein the point of attachment is on the cycloalkyl ring, and in such instances, the number of carbons continue to designate the number of carbons in the cycloalkyl ring system. Each instance of a cycloalkyl group may be independently optionally substituted, i.e., unsubstituted (an “unsubstituted cycloalkyl”) or substituted (a “substituted cycloalkyl”) with one or more substituents. In certain embodiments, the cycloalkyl group is unsubstituted C₃-C₁₀ cycloalkyl. In certain embodiments, the cycloalkyl group is a substituted C₃-C₁₀ cycloalkyl.

In some embodiments, “cycloalkyl” is a monocyclic, saturated cycloalkyl group having from 3 to 10 ring carbon atoms (“C₃-C₁₀ cycloalkyl”). In some embodiments, a cycloalkyl group has 3 to 8 ring carbon atoms (“C₃-C₈ cycloalkyl”). In some embodiments, a cycloalkyl group has 3 to 6 ring carbon atoms (“C₃-C₆ cycloalkyl”). In some embodiments, a cycloalkyl group has 5 to 6 ring carbon atoms (“C₅-C₆ cycloalkyl”). In some embodiments, a cycloalkyl group has 5 to 10 ring carbon atoms (“C₅-C₁₀ cycloalkyl”). Examples of C₅-C₆ cycloalkyl groups include cyclopentyl (C₅) and cyclohexyl (C₅). Examples of C₃-C₆ cycloalkyl groups include the aforementioned C₅-C₆ cycloalkyl groups as well as cyclopropyl (C₃) and cyclobutyl (C₄). Examples of C₃-C₈ cycloalkyl groups include the aforementioned C₃-C₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is unsubstituted C₃-C₁₀ cycloalkyl. In certain embodiments, the cycloalkyl group is substituted C₃-C₁₀ cycloalkyl.

“Heterocyclyl” or “heterocyclic” refers to a radical of a 3- to 10-membered non-aromatic ring system having ring carbon atoms and 1 to 4 ring heteroatoms, wherein each heteroatom is independently selected from nitrogen, oxygen, sulfur, boron, phosphorus, and silicon (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more cycloalkyl groups wherein the point of attachment is either on the cycloalkyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. A heterocyclyl group may be described as, e.g., a 3-7-membered heterocyclyl, wherein the term “membered” refers to the non-hydrogen ring atoms, i.e., carbon, nitrogen, oxygen, sulfur, boron, phosphorus, and silicon, within the moiety. Each instance of heterocyclyl may be independently optionally substituted, i.e., unsubstituted (an “unsubstituted heterocyclyl”) or substituted (a “substituted heterocyclyl”) with one or more substituents. In certain embodiments, the heterocyclyl group is unsubstituted 3-10 membered heterocyclyl. In certain embodiments, the heterocyclyl group is substituted 3-10 membered heterocyclyl.

In some embodiments, a heterocyclyl group is a 5-10 membered non-aromatic ring system having ring carbon atoms and 1-4 ring heteroatoms, wherein each heteroatom is independently selected from nitrogen, oxygen, sulfur, boron, phosphorus, and silicon (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has one ring heteroatom selected from nitrogen, oxygen, and sulfur.

Exemplary 3-membered heterocyclyl groups containing one heteroatom include, without limitation, azirdinyl, oxiranyl, thiorenyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

Particular examples of heterocyclyl groups are shown in the following illustrative examples:

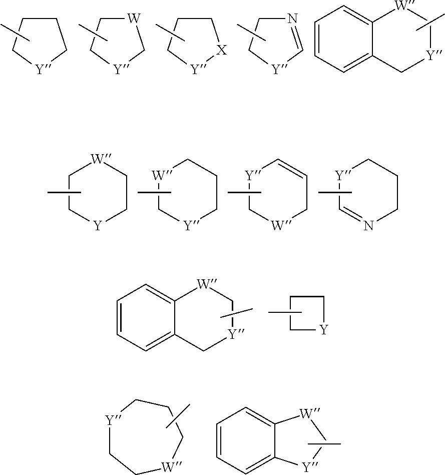

wherein each W″ is selected from CR⁶⁷, C(R⁶⁷)₂, NR⁶⁷, O, and S; and each Y″ is selected from NR⁶⁷, O, and S; and R⁶⁷ is independently hydrogen, C₁-C₈ alkyl, C₃-C₁₀ cycloalkyl, 4-10 membered heterocyclyl, C₆-C₁₀ aryl, and 5-10-membered heteroaryl. These heterocyclyl rings may be optionally substituted with one or more groups selected from the group consisting of acyl, acylamino, acyloxy, alkoxy, alkoxycarbonyl, alkoxycarbonylamino, amino, substituted amino, aminocarbonyl (e.g., amido), aminocarbonylamino, aminosulfonyl, sulfonylamino, aryl, aryloxy, azido, carboxyl, cyano, cycloalkyl, halogen, hydroxy, keto, nitro, thiol, —S-alkyl, —S-aryl, —S(O)-alkyl, —S(O)-aryl, —S(O)₂-alkyl, and —S(O)₂-aryl. Substituting groups include carbonyl or thiocarbonyl which provide, for example, lactam and urea derivatives.

“Nitrogen-containing heterocyclyl” group means a 4- to 7-membered non-aromatic cyclic group containing at least one nitrogen atom, for example, but without limitation, morpholine, piperidine (e.g. 2-piperidinyl, 3-piperidinyl and 4-piperidinyl), pyrrolidine (e.g. 2-pyrrolidinyl and 3-pyrrolidinyl), azetidine, pyrrolidone, imidazoline, imidazolidinone, 2-pyrazoline, pyrazolidine, piperazine, and N-alkyl piperazines such as N-methyl piperazine. Particular examples include azetidine, piperidone and piperazone.

“Amino” refers to the radical —NR⁷⁰R⁷¹, wherein R⁷⁰ and R⁷¹ are each independently hydrogen, C₁-C₈ alkyl, C₃-C₁₀ cycloalkyl, 4-10 membered heterocyclyl, C₆-C₁₀ aryl, and 5-10-membered heteroaryl. In some embodiments, amino refers to NH₂.

“Cyano” refers to the radical —CN.

“Hydroxy” refers to the radical —OH.

Alkyl, alkenyl, alkynyl, cycloalkyl, heterocyclyl, aryl, and heteroaryl groups, as defined herein, are optionally substituted (e.g., “substituted” or “unsubstituted” alkyl, “substituted” or “unsubstituted” alkenyl, “substituted” or “unsubstituted” alkynyl, “substituted” or “unsubstituted” cycloalkyl, “substituted” or “unsubstituted” heterocyclyl, “substituted” or “unsubstituted” aryl or “substituted” or “unsubstituted” heteroaryl group). In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, such as any of the substituents described herein that result in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

A “counterion” or “anionic counterion” is a negatively charged group associated with a cationic quaternary amino group in order to maintain electronic neutrality. Exemplary counterions include halide ions (e.g., F⁻, Cl⁻, Br⁻, I⁻), NO₃⁻, ClO₄⁻, OH⁻, H₂PO₄⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), and carboxylate ions (e.g., acetate, ethanoate, propanoate, benzoate, glycerate, lactate, tartrate, glycolate, and the like).

The term “pharmaceutically acceptable salts” is meant to include salts of the active compounds that are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, e.g., Berge et al, Journal of Pharmaceutical Science 66: 1-19 (1977)). Certain specific compounds of the present invention contain both basic and acidic functionalities that allow the compounds to be converted into either base or acid addition salts. Other pharmaceutically acceptable carriers known to those of skill in the art are suitable for the present invention. Salts tend to be more soluble in aqueous or other protonic solvents that are the corresponding free base forms. In other cases, the preparation may be a lyophilized powder in a first buffer, e.g., in 1 mM-50 mM histidine, 0.1%-2% sucrose, 2%-7% mannitol at a pH range of 4.5 to 5.5, that is combined with a second buffer prior to use.

Thus, the compounds of the present invention may exist as salts, such as with pharmaceutically acceptable acids. The present invention includes such salts. Examples of such salts include hydrochlorides, hydrobromides, sulfates, methanesulfonates, nitrates, maleates, acetates, citrates, fumarates, tartrates (e.g., (+)-tartrates, (−)-tartrates, or mixtures thereof including racemic mixtures), succinates, benzoates, and salts with amino acids such as glutamic acid. These salts may be prepared by methods known to those skilled in the art.

The neutral forms of the compounds are preferably regenerated by contacting the salt with a base or acid and isolating the parent compound in the conventional manner. The parent form of the compound differs from the various salt forms in certain physical properties, such as solubility in polar solvents.

In addition to salt forms, the present invention provides compounds, which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

As used herein, the term “salt” refers to acid or base salts of the compounds used in the methods of the present invention. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts.

Certain compounds of the present invention possess asymmetric carbon atoms (optical or chiral centers) or double bonds; the enantiomers, racemates, diastereomers, tautomers, geometric isomers, stereoisomeric forms that may be defined, in terms of absolute stereochemistry, as (R)- or (S)- or, as (D)- or (L)- for amino acids, and individual isomers are encompassed within the scope of the present invention. The compounds of the present invention do not include those which are known in art to be too unstable to synthesize and/or isolate. The present invention is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this invention may exist in tautomeric forms, all such tautomeric forms of the compounds being within the scope of the invention.

The terms “treating” or “treatment” refers to any indicia of success in the treatment or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. For example, certain methods herein treat cancer (e.g. pancreatic cancer, breast cancer, multiple myeloma, cancers of secretory cells), neurodegenerative diseases (e.g. Alzheimer's disease, Parkinson's disease, frontotemporal dementia), leukodystrophies (e.g., vanishing white matter disease, childhood ataxia with CNS hypo-myelination), postsurgical cognitive dysfunction, traumatic brain injury, stroke, spinal cord injury, intellectual disability syndromes, inflammatory diseases, musculoskeletal diseases, metabolic diseases, or diseases or disorders associated with impaired function of eIF2B or components in a signal transduction or signaling pathway including the ISR and decreased eIF2 pathway activity). For example certain methods herein treat cancer by decreasing or reducing or preventing the occurrence, growth, metastasis, or progression of cancer or decreasing a symptom of cancer; treat neurodegeneration by improving mental wellbeing, increasing mental function, slowing the decrease of mental function, decreasing dementia, delaying the onset of dementia, improving cognitive skills, decreasing the loss of cognitive skills, improving memory, decreasing the degradation of memory, decreasing a symptom of neurodegeneration or extending survival; treat vanishing white matter disease by reducing a symptom of vanishing white matter disease or reducing the loss of white matter or reducing the loss of myelin or increasing the amount of myelin or increasing the amount of white matter; treat childhood ataxia with CNS hypo-myelination by decreasing a symptom of childhood ataxia with CNS hypo-myelination or increasing the level of myelin or decreasing the loss of myelin; treat an intellectual disability syndrome by decreasing a symptom of an intellectual disability syndrome, treat an inflammatory disease by treating a symptom of the inflammatory disease; treat a musculoskeletal disease by treating a symptom of the musculoskeletal disease; or treat a metabolic disease by treating a symptom of the metabolic disease. Symptoms of a disease, disorder, or condition described herein (e.g., cancer, a neurodegenerative disease, a leukodystrophy, an inflammatory disease, a musculoskeletal disease, a metabolic disease, or a condition or disease associated with impaired function of eIF2B or components in a signal transduction pathway including the eIF2 pathway, eIF2α phosphorylation. or ISR pathway) would be known or may be determined by a person of ordinary skill in the art. The term “treating” and conjugations thereof, include prevention of an injury, pathology, condition, or disease (e.g. preventing the development of one or more symptoms of a disease, disorder, or condition described herein).

An “effective amount” is an amount sufficient to accomplish a stated purpose (e.g. achieve the effect for which it is administered, treat a disease, reduce enzyme activity, increase enzyme activity, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

A “reduction” of a symptom or symptoms (and grammatical equivalents of this phrase) means decreasing of the severity or frequency of the symptom(s), or elimination of the symptom(s).

The term “associated” or “associated with” in the context of a substance or substance activity or function associated with a disease (e.g., a disease or disorder described herein, e.g., cancer, a neurodegenerative disease, a leukodystrophy, an inflammatory disease, a musculoskeletal disease, a metabolic disease, or a disease or disorder associated with impaired function of eIF2B or components in a signal transduction pathway including the eIF2 pathway, eIF2α phosphorylation. or ISR pathway) means that the disease is caused by (in whole or in part), or a symptom of the disease is caused by (in whole or in part) the substance or substance activity or function. For example, a symptom of a disease or condition associated with an impaired function of the eIF2B may be a symptom that results (entirely or partially) from a decrease in eIF2B activity (e.g. decrease in eIF2B activity or levels, increase in eIF2α phosphorylation or activity of phosphorylated eIF2α or reduced eIF2 activity or increase in activity of phosphorylated eIF2α signal transduction or the ISR signalling pathway). As used herein, what is described as being associated with a disease, if a causative agent, could be a target for treatment of the disease. For example, a disease associated with decreased eIF2 activity or eIF2 pathway activity, may be treated with an agent (e.g., compound as described herein) effective for increasing the level or activity of eIF2 or eIF2 pathway or a decrease in phosphorylated eIF2α activity or the ISR pathway. For example, a disease associated with phosphorylated eIF2α may be treated with an agent (e.g., compound as described herein) effective for decreasing the level of activity of phosphorylated eIF2α or a downstream component or effector of phosphorylated eIF2α. For example, a disease associated with eIF2α may be treated with an agent (e.g., compound as described herein) effective for increasing the level of activity of eIF2 or a downstream component or effector of eIF2.

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects.

“Contacting” is used in accordance with its plain ordinary meaning and refers to the process of allowing at least two distinct species (e.g. chemical compounds including biomolecules, or cells) to become sufficiently proximal to react, interact or physically touch. It should be appreciated, however, that the resulting reaction product can be produced directly from a reaction between the added reagents or from an intermediate from one or more of the added reagents which can be produced in the reaction mixture. The term “contacting” may include allowing two species to react, interact, or physically touch, wherein the two species may be a compound as described herein and a protein or enzyme (e.g. eIF2B, eIF2α, or a component of the eIF2 pathway or ISR pathway). In some embodiments contacting includes allowing a compound described herein to interact with a protein or enzyme that is involved in a signaling pathway (e.g. eIF2B, eIF2α, or a component of the eIF2 pathway or ISR pathway).

As defined herein, the term “inhibition”, “inhibit”, “inhibiting” and the like in reference to a protein-inhibitor (e.g., antagonist) interaction means negatively affecting (e.g., decreasing) the activity or function of the protein relative to the activity or function of the protein in the absence of the inhibitor. In some embodiments, inhibition refers to reduction of a disease or symptoms of disease. In some embodiments, inhibition refers to a reduction in the activity of a signal transduction pathway or signaling pathway. Thus, inhibition includes, at least in part, partially or totally blocking stimulation, decreasing, preventing, or delaying activation, or inactivating, desensitizing, or down-regulating signal transduction or enzymatic activity or the amount of a protein. In some embodiments, inhibition refers to a decrease in the activity of a signal transduction pathway or signaling pathway (e.g., eIF2B, eIF2α, or a component of the eIF2 pathway, pathway activated by eIF2α phosphorylation, or ISR pathway). Thus, inhibition may include, at least in part, partially or totally decreasing stimulation, decreasing or reducing activation, or inactivating, desensitizing, or down-regulating signal transduction or enzymatic activity or the amount of a protein increased in a disease (e.g. eIF2B, eIF2α, or a component of the eIF2 pathway or ISR pathway, wherein each is associated with cancer, a neurodegenerative disease, a leukodystrophy, an inflammatory disease, a musculoskeletal disease, or a metabolic disease). Inhibition may include, at least in part, partially or totally decreasing stimulation, decreasing or reducing activation, or deactivating, desensitizing, or down-regulating signal transduction or enzymatic activity or the amount of a protein (e.g. eIF2B, eIF2α, or component of the eIF2 pathway or ISR pathway) that may modulate the level of another protein or increase cell survival (e.g., decrease in phosphorylated eIF2α pathway activity may increase cell survival in cells that may or may not have an increase in phosphorylated eIF2α pathway activity relative to a non-disease control or decrease in eIF2α pathway activity may increase cell survival in cells that may or may not have an increase in eIF2α pathway activity relative to a non-disease control).

As defined herein, the term “activation”, “activate”, “activating” and the like in reference to a protein-activator (e.g. agonist) interaction means positively affecting (e.g. increasing) the activity or function of the protein (e.g. eIF2B, eIF2α, or component of the eIF2 pathway or ISR pathway) relative to the activity or function of the protein in the absence of the activator (e.g. compound described herein). In some embodiments, activation refers to an increase in the activity of a signal transduction pathway or signaling pathway (e.g. eIF2B, eIF2α, or component of the eIF2 pathway or ISR pathway). Thus, activation may include, at least in part, partially or totally increasing stimulation, increasing or enabling activation, or activating, sensitizing, or up-regulating signal transduction or enzymatic activity or the amount of a protein decreased in a disease (e.g. level of eIF2B, eIF2α, or component of the eIF2 pathway or ISR pathway associated with cancer, a neurodegenerative disease, a leukodystrophy, an inflammatory disease, a musculoskeletal disease, or a metabolic disease). Activation may include, at least in part, partially or totally increasing stimulation, increasing or enabling activation, or activating, sensitizing, or up-regulating signal transduction or enzymatic activity or the amount of a protein (e.g., eIF2B, eIF2α, or component of the eIF2 pathway or ISR pathway) that may modulate the level of another protein or increase cell survival (e.g., increase in eIF2α activity may increase cell survival in cells that may or may not have a reduction in eIF2α activity relative to a non-disease control).

The term “modulation” refers to an increase or decrease in the level of a target molecule or the function of a target molecule. In some embodiments, modulation of eIF2B, eIF2α, or a component of the eIF2 pathway or ISR pathway may result in reduction of the severity of one or more symptoms of a disease associated with eIF2B, eIF2α, or a component of the eIF2 pathway or ISR pathway (e.g., cancer, a neurodegenerative disease, a leukodystrophy, an inflammatory disease, a musculoskeletal disease, or a metabolic disease) or a disease that is not caused by eIF2B, eIF2α, or a component of the eIF2 pathway or ISR pathway but may benefit from modulation of eIF2B, eIF2α, or a component of the eIF2 pathway or ISR pathway (e.g., decreasing in level or level of activity of eIF2B, eIF2α or a component of the eIF2 pathway).

The term “modulator” as used herein refers to modulation of (e.g., an increase or decrease in) the level of a target molecule or the function of a target molecule. In embodiments, a modulator of eIF2B, eIF2α, or component of the eIF2 pathway or ISR pathway is an anti-cancer agent. In embodiments, a modulator of eIF2B, eIF2α, or component of the eIF2 pathway or ISR pathway is a neuroprotectant. In embodiments, a modulator of eIF2B, eIF2α, or component of the eIF2 pathway or ISR pathway is a memory enhancing agent. In embodiments, a modulator of eIF2B, eIF2α, or component of the eIF2 pathway or ISR pathway is a memory enhancing agent (e.g., a long term memory enhancing agent). In embodiments, a modulator of eIF2B, eIF2α, or component of the eIF2 pathway or ISR pathway is an anti-inflammatory agent. In some embodiments, a modulator of eIF2B, eIF2α, or component of the eIF2 pathway or ISR pathway is a pain-relieving agent.

“Patient” or “subject in need thereof refers to a living organism suffering from or prone to a disease or condition that can be treated by administration of a compound or pharmaceutical composition, as provided herein. Non-limiting examples include humans, other mammals, bovines, rats, mice, dogs, monkeys, goat, sheep, cows, deer, and other non-mammalian animals. In some embodiments, a patient is human. In some embodiments, a patient is a domesticated animal. In some embodiments, a patient is a dog. In some embodiments, a patient is a parrot. In some embodiments, a patient is livestock animal. In some embodiments, a patient is a mammal. In some embodiments, a patient is a cat. In some embodiments, a patient is a horse. In some embodiments, a patient is bovine. In some embodiments, a patient is a canine. In some embodiments, a patient is a feline. In some embodiments, a patient is an ape. In some embodiments, a patient is a monkey. In some embodiments, a patient is a mouse. In some embodiments, a patient is an experimental animal. In some embodiments, a patient is a rat. In some embodiments, a patient is a hamster. In some embodiments, a patient is a test animal. In some embodiments, a patient is a newborn animal. In some embodiments, a patient is a newborn human. In some embodiments, a patient is a newborn mammal. In some embodiments, a patient is an elderly animal. In some embodiments, a patient is an elderly human. In some embodiments, a patient is an elderly mammal. In some embodiments, a patient is a geriatric patient.

“Disease”, “disorder” or “condition” refers to a state of being or health status of a patient or subject capable of being treated with a compound, pharmaceutical composition, or method provided herein. In some embodiments, the compounds and methods described herein comprise reduction or elimination of one or more symptoms of the disease, disorder, or condition, e.g., through administration of a compound of Formula (I) or a pharmaceutically acceptable salt thereof.

The term “signaling pathway” as used herein refers to a series of interactions between cellular and optionally extra-cellular components (e.g. proteins, nucleic acids, small molecules, ions, lipids) that conveys a change in one component to one or more other components, which in turn may convey a change to additional components, which is optionally propagated to other signaling pathway components.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

As used herein, the term “administering” means oral administration, administration as a suppository, topical contact, intravenous, parenteral, intraperitoneal, intramuscular, intralesional, intrathecal, intracranial, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arterial, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies (e.g., anti-cancer agent, chemotherapeutic, or treatment for a neurodegenerative disease). The compound of the invention can be administered alone or can be coadministered to the patient. Coadministration is meant to include simultaneous or sequential administration of the compound individually or in combination (more than one compound or agent). Thus, the preparations can also be combined, when desired, with other active substances (e.g. to reduce metabolic degradation).

The term “eIF2B” as used herein refers to the heteropentameric eukaryotic translation initiation factor 2B. eIF2B is composed of five subunits: eIF2B1, eIF2B2, eIF2B3, eIF2B4 and eIF2B5. eIF2B1 refers to the protein associated with Entrez gene 1967, OMIM 606686, Uniprot Q14232, and/or RefSeq (protein) NP_001405. eIF2B2 refers to the protein associated with Entrez gene 8892, OMIM 606454, Uniprot P49770, and/or RefSeq (protein) NP_055054. eIF2B3 refers to the protein associated with Entrez gene 8891, OMIM 606273, Uniprot Q9NR50, and/or RefSeq (protein) NP_065098. eIF2B4 refers to the protein associated with Entrez gene 8890, OMIM 606687, Uniprot Q9UI10, and/or RefSeq (protein) NP_751945. eIF2B5 refers to the protein associated with Entrez gene 8893, OMIM 603945, Uniprot Q13144, and/or RefSeq (protein) NP_003898.

The terms “eIF2alpha,” “eIF2α,” or “eIF2α” are interchangeable and refer to the protein “eukaryotic translation initiation factor 2 alpha subunit eIF2S1”. In embodiments, “eIF2alpha”, “eIF2α” or “eIF2α” refer to the human protein. Included in the terms “eIF2alpha”, “eIF2α” or “eIF2α” are the wild type and mutant forms of the protein. In embodiments, “eIF2alpha”, “eIF2α” or “eIF2α” refer to the protein associated with Entrez Gene 1965, OMIM 603907, UniProt P05198, and/or RefSeq (protein) NP_004085. In embodiments, the reference numbers immediately above refer to the protein and associated nucleic acids known as of the date of filing of this application.

### Compounds

Disclosed herein, for example, is a compound of Formula (I):

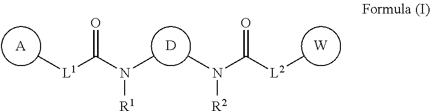

or a pharmaceutically acceptable salt, solvate, hydrate, tautomer, N-oxide, or stereoisomer thereof, wherein:
D is a bridged bicyclic cycloalkyl, bridged bicyclic heterocyclyl, or cubanyl, wherein each bridged bicyclic cycloalkyl, bridged bicyclic heterocyclyl, or cubanyl is optionally substituted on one or more available carbons with 1-4 R^{X}; and wherein if the bridged bicyclic heterocyclyl contains a substitutable nitrogen moiety, the substitutable nitrogen may be optionally substituted by R^{N1};
L¹ is a bond, C₁-C₆ alkylene, 2-7 membered heteroalkylene, —NR^{N2}—, or —O—, wherein C₁-C₆ alkylene or 2-7 membered heteroalkylene is optionally substituted with 1-5 R^{L1};
L² is a bond, C₁-C₆ alkylene, or 2-7 membered heteroalkylene, wherein C₁-C₆ alkylene or 2-7 membered heteroalkylene is optionally substituted with 1-5 R^{L2};
R¹ is hydrogen or C₁-C₆ alkyl;
R² is hydrogen or C₁-C₆ alkyl;
W is a 8-10 membered, partially unsaturated, fused bicyclic ring moiety comprising a 5-6 membered heterocyclyl fused to a phenyl or 5-6-membered heteroaryl; wherein the heterocyclyl may be optionally substituted on one or more available carbons with 1-4 R^{W1}; and wherein the phenyl or heteroaryl may optionally be substituted on one or more available unsaturated carbons with 1-4 R^{W2}; and wherein if the heterocyclyl contains a substitutable nitrogen moiety, the substitutable nitrogen may optionally be substituted with R^{N3};
A is phenyl or 5-6-membered heteroaryl, wherein phenyl or 5-6-membered heteroaryl is optionally substituted on one or more available carbons with 1-5 R^{Y}; and wherein if the 5-6-membered heteroaryl contains a substitutable nitrogen moiety, the substitutable nitrogen may be optionally substituted by R^{N4};
each R^{L1} is independently selected from the group consisting of hydrogen, C₁-C₆ alkyl, hydroxy-C₁-C₆ alkyl, halo-C₁-C₆ alkyl, amino-C₁-C₆ alkyl, cyano-C₁-C₆ alkyl, oxo, halo, cyano, —OR^{A}, —NR^{B}R^{C}, —NR^{B}C(O)R^{D}, —C(O)NR^{B}R^{C}, —C(O)R^{D}, —C(O)OH, —C(O)OR^{D}, —SR^{E}, —S(O)R^{D}, and —S(O)₂R^{D};
each R^{L2} is independently selected from the group consisting of hydrogen, C₁-C₆ alkyl, hydroxy-C₁-C₆ alkyl, halo-C₁-C₆ alkyl, amino-C₁-C₆ alkyl, cyano-C₁-C₆ alkyl, oxo, halo, cyano, —OR^{A}, —NR^{B}R^{C}, —NR^{B}C(O)R^{D}, —C(O)NR^{B}R^{C}, —C(O)R^{D}, —C(O)OH, —C(O)OR^{D}, —SR^{E}, —S(O)R^{D}, and —S(O)₂R^{D};
R^{N1} is selected from the group consisting of hydrogen, C₁-C₆ alkyl, hydroxy-C₂-C₆ alkyl, halo-C₂-C₆ alkyl, amino-C₂-C₆ alkyl, cyano-C₂-C₆ alkyl, —C(O)NR^{B}R^{C}, —C(O)R^{D}, —C(O)OR^{D}, and —S(O)₂R^{D};
R^{N2} is selected from the group consisting of hydrogen, C₁-C₆ alkyl, hydroxy-C₂-C₆ alkyl, halo-C₂-C₆ alkyl, amino-C₂-C₆ alkyl, cyano-C₂-C₆ alkyl, —C(O)NR^{B}R^{C}, —C(O)R^{D}, —C(O)OR^{D}, and —S(O)₂R^{D};
R^{N3} is selected from the group consisting of hydrogen, C₁-C₆ alkyl, hydroxy-C₂-C₆ alkyl, C₁-C₆ alkyl-C₁-C₆ cycloalkyl, C₁-C₆ alkenyl, —C(O)—C₁-C₆ alkyl, —C(O)—C₁-C₆ cycloalkyl, C₁-C₆ alkyl-CO₂H, C₁-C₆ alkyl-CO₂—C₁-C₆ alkyl, —C(O)—C₁-C₃ alkyl-O—C₁-C₃ alkyl-O—C₁-C₃ alkyl, —C(O)-phenyl, —C(O)-heteroaryl, —C(O)-heterocyclyl, —S—C₁-C₆ alkyl, —S(O)₂—C₁-C₆ alkyl, —S(O)₂-phenyl, —S(O)₂-heteroaryl, —C(O)NR^{B}R^{C} and —C(O)OR^{D};
   wherein C₁-C₆ alkyl, hydroxy-C₂-C₆ alkyl, C₁-C₆ alkyl-C₁-C₆ cycloalkyl, C₁-C₆ alkenyl, C(O)—C₁-C₆ alkyl, —C(O)—C₁-C₆ cycloalkyl, C₁-C₆ alkyl-CO₂H, C₁-C₆ alkyl-CO₂—C₁-C₆ alkyl, —C(O)-heterocyclyl, —S—C₁-C₆ alkyl and —S(O)₂—C₁-C₆ alkyl may optionally be substituted by one or more substituents each independently selected from the group consisting of fluoro, hydroxyl, C₁-C₆ alkoxy, C₁-C₆ alkyl (optionally substituted by one, two or three fluorine atoms) and S(O)_{w}C₁₋₆ alkyl (wherein w is 0, 1 or 2); and
   wherein —C(O)-phenyl, —C(O)-heteroaryl, —S(O)₂-phenyl and —S(O)₂-heteroaryl may optionally be substituted by one or more substituents each independently selected from the group consisting of halogen, hydroxyl, C₁-C₆ alkyl (optionally substituted by one, two or three fluorine atoms), C₁-C₆ alkoxy (optionally substituted by one, two or three fluorine atoms), S(O₂)NR^{B}R^{C} and SO₂F;
R^{N4} is selected from the group consisting of hydrogen, C₁-C₆ alkyl, hydroxy-C₂-C₆ alkyl, halo-C₂-C₆ alkyl, amino-C₂-C₆ alkyl, cyano-C₂-C₆ alkyl, C₃-C₆ cycloalkyl, phenyl, 5-6-membered heteroaryl, —C(O)NR^{B}R^{C}, —C(O)R^{D}, —C(O)OR^{D}, and —S(O)₂R^{D};
   wherein C₃-C₆ cycloalkyl, phenyl, and 5-6-membered heteroaryl may optionally be substituted by one or more substituents each independently selected from the group consisting of halo, C₁-C₆ alkyl (optionally substituted by one, two or three fluorine atoms), and C₁-C₆ alkoxy (optionally substituted by one, two or three fluorine atoms).
each R^{W1} is independently selected from the group consisting of hydrogen, C₁-C₆ alkyl (optionally substituted by —CO₂H), hydroxy-C₁-C₆ alkyl, hydroxy-C₂-C₆ alkyl-O—, halo-C₁-C₆ alkyl, amino-C₁-C₆ alkyl, cyano-C₁-C₆ alkyl, oxo, C═NOH, halo, cyano, —OR^{A}, —NR^{B}R^{C}, —NR^{B}R^{CC}, —R^{B}C(O)R^{D}, —C(O)NR^{B}R^{C}, —C(O)R^{D}, —C(O)OH, —C(O)OR^{D}, —SR^{E}, —S(O)R^{D}, and —S(O)₂R^{D};
each R^{W2} is independently selected from the group consisting of hydrogen, C₁-C₆ alkyl, hydroxy-C₁-C₆ alkyl, hydroxy-C₂-C₆ alkyl-O—, halo-C₁-C₆ alkyl, halo-C₁-C₆ alkoxy, amino-C₁-C₆ alkyl, cyano-C₁-C₆ alkyl, halo, cyano, —OR^{A}, —NR^{B}R^{C}, —N^{B}C(O)R^{D}, —C(O)NR^{B}R^{C}, —C(O)R^{D}, —C(O)OH, —C(O)OR^{D}, —S(R^{F})ₘ, —S(O)R^{D}, and —S(O)₂R^{D}; or
2 R^{W2} groups on adjacent atoms, together with the atoms to which they are attached, form a 3-7-membered fused cycloalkyl, 3-7-membered fused heterocyclyl, fused aryl, or 5-6 membered fused heteroaryl, each of which is optionally substituted with 1-5 R^{X};
each R^{X} is independently selected from the group consisting of hydrogen, C₁-C₆ alkyl, hydroxy-C₁-C₆ alkyl, halo-C₁-C₆ alkyl, amino-C₁-C₆ alkyl, cyano-C₁-C₆ alkyl, oxo, halo, cyano, —OR^{A}, —NR^{B}R^{C}, —N^{B}C(O)R^{D}, —C(O)NR^{B}R^{C}, —C(O)R^{D}, —C(O)OH, —C(O)OR^{D}, —SR^{E}, —S(O)R^{D}, and —S(O)₂R^{D};
each R^{Y} is independently selected from the group consisting of hydrogen, C₁-C₆ alkyl, hydroxy-C₁-C₆ alkyl, halo-C₁-C₆ alkyl, halo-C₁-C₆ alkoxy, amino-C₁-C₆ alkyl, cyano-C₁-C₆ alkyl, C₃-C₆ cycloalkyl, 3-7 membered heterocyclyl, halo-C₁-C₆ alkyl-3-7 membered heterocyclyl, halo, cyano, —OR^{A}, —NR^{B}R^{C}, —N^{B}C(O)R^{D}, —C(O)NR^{B}R^{C}, —C(O)R^{D}, —C(O)OH, —C(O)OR^{D}, —S(R^{F})ₘ, —S(O)R^{D}, —S(O)₂R^{D}, and G¹; or
2 R^{Y} groups on adjacent atoms, together with the atoms to which they are attached form a 3-7-membered fused cycloalkyl, 3-7-membered fused heterocyclyl, fused aryl, or 5-6 membered fused heteroaryl, each of which is optionally substituted with 1-5 R^{X};
each G¹ is independently 3-7-membered cycloalkyl, 3-7-membered heterocyclyl, aryl, or 5-6-membered heteroaryl, wherein each 3-7-membered cycloalkyl, 3-7-membered heterocyclyl, aryl, or 5-6-membered heteroaryl is optionally substituted with 1-3 R^{Z};
each R^{Z} is independently selected from the group consisting of C₁-C₆ alkyl, hydroxy-C₁-C₆ alkyl, halo-C₁-C₆ alkyl, halo, cyano, —OR^{A}, —NR^{B}R^{C}, —N^{B}C(O)R^{D}, —C(O)NR^{B}R^{C}, —C(O)R^{D}, —C(O)OH, —C(O)OR^{D}, and —S(O)₂R^{D};
R^{A} is, at each occurrence, independently hydrogen, C₁-C₆ alkyl, halo-C₁-C₆ alkyl, —C(O)NR^{B}R^{C}, —C(O)R^{D}, or —C(O)OR^{D};
each of R^{B} and R^{C} is independently hydrogen or C₁-C₆ alkyl;
R^{B} and R^{C} together with the atom to which they are attached form a 3-7-membered heterocyclyl ring optionally substituted with 1-3 R^{Z};
each R^{CC} is independently selected from the group consisting of hydroxy-C₁-C₆ alkyl, halo-C₁-C₆ alkyl, C₁-C₆ alkyl-CO₂H, C₁-C₆ alkyl-CO₂—C₁-C₆ alkyl, C(O) C₁-C₆ alkyl, S(O)₂—C₁-C₆ alkyl, 3-6-membered cycloalkyl and 4-6-membered heterocyclyl; wherein 3-6-membered cycloalkyl and 4-6-membered heterocyclyl may optionally be substituted by one or more substituents each independently selected from the group consisting of C₁-C₆ alkyl, hydroxy-C₁-C₆ alkyl, halo-C₁-C₆ alkyl, hydroxyl, halo and —C(O)OH;
each R^{D} is independently C₁-C₆ alkyl or halo-C₁-C₆ alkyl;
each R^{E} is independently hydrogen, C₁-C₆ alkyl, or halo-C₁-C₆ alkyl;
each R^{F} is independently hydrogen, C₁-C₆ alkyl, or halo; and
m is 1 when R^{F} is hydrogen or C₁-C₆ alkyl, or 5 when R^{F} is halo.

In some embodiments, D is a bridged bicyclic cycloalkyl or bridged bicyclic heterocyclyl, wherein each bridged bicyclic cycloalkyl or bridged bicyclic heterocyclyl may optionally be substituted by 1-4 R^{X}. For example, in some embodiments D is a bridged bicyclic 5-8 membered cycloalkyl or a bridged bicyclic 5-8-membered heterocyclyl, wherein each bridged bicyclic 5-8-membered cycloalkyl or bridged bicyclic 5-8-membered heterocyclyl may optionally be substituted by 1-4 R^{X}.

For example, in some embodiments D is bicyclo[1.1.1]pentane, bicyclo[2.2.1]heptane, bicyclo[2.1.1]hexane, bicyclo[2.2.2]octane, bicyclo[3.2.1]octane, 7-oxabicyclo[2.2.1]heptane, 2-oxabicyclo[2.2.2]octane, or 2-azabicyclo[2.2.2]octane, each of which may optionally be substituted by 1-4 R^{X} groups. In some embodiments, or example, D is

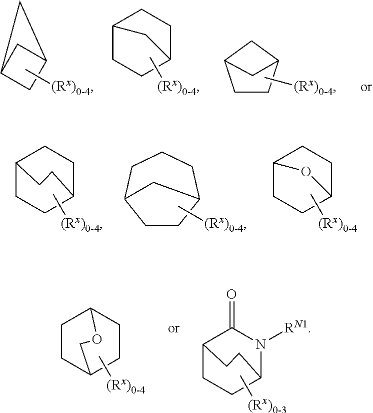

In further embodiments, D is

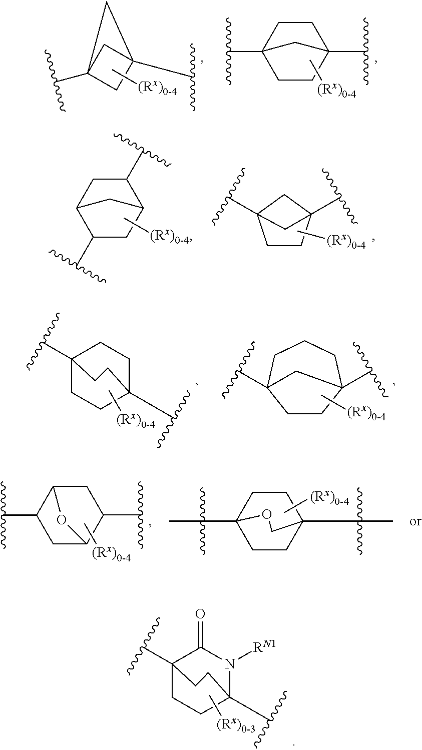

For example, in certain embodiments D is

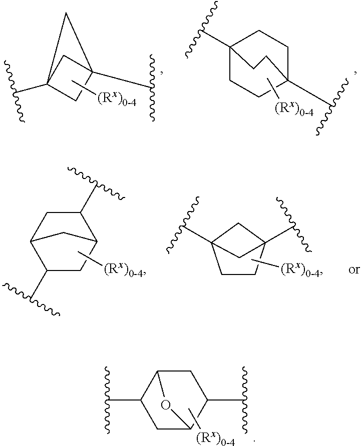

In some embodiments D is substituted with 0 R^{X}. For example, in certain embodiments D is

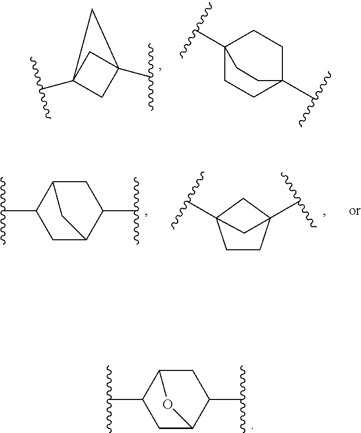 [SMILES: *C12CC(*)(C1)C2 ; *C12CCC(*)(CC1)CC2 ; *C1CC2CC1CC2* ; *C12CCC(*)(C1)C2 ; *C1CC2OC1CC2*]

In some embodiments, D is

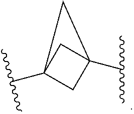 [SMILES: *C12CC(*)(C1)C2]

In other embodiments, D is

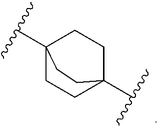 [SMILES: *C12CCC(*)(CC1)CC2]

In some embodiments, D is substituted with 1 or 2 R^{X}. For example, in certain embodiments D is

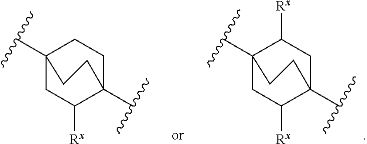

In some embodiments, each R^{X} is independently selected from the group consisting of oxo, —OH, —C(O)OH, —C(O)OR^{D}, halo, and hydroxy-C₁-C₆ alkyl.

In some embodiments, L¹ is a bond, 2-7 membered heteroalkylene, —NR^{N2}—, or —O—, wherein 2-7 membered heteroalkylene is optionally substituted by 1-5 R^{L1}. In other embodiments, L¹ is a bond, 2-7 membered heteroalkylene, —NR^{N2}—, or —O—, wherein 2-7 membered heteroalkylene is substituted by 0 R^{L1}. For example, in certain embodiments L¹ is selected from a bond and CH₂O—*, wherein “-*” indicates the attachment point to A.

In some embodiments, R¹ is hydrogen or CH₃. In other embodiments, R² is hydrogen or CH₃.

In some embodiments, A is selected from the group consisting of phenyl, pyrazinyl, isoxazolyl, pyrimidinyl, oxazolyl, thiazolyl and pyridyl, each of which is optionally substituted with 1-2 R^{Y} groups; or A is pyrazolyl optionally substituted by R^{N4}. For example, in certain embodiments A is selected from the group consisting of:

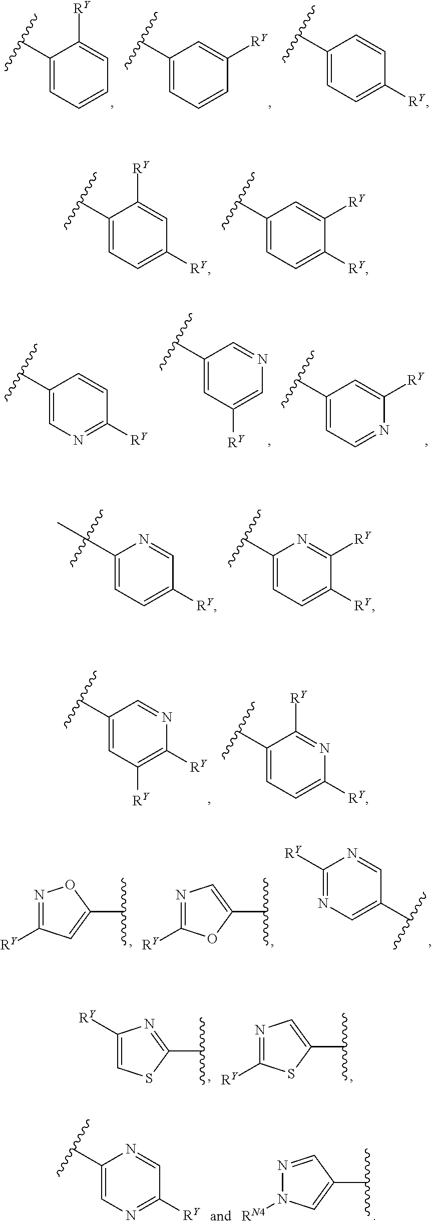

In some embodiments, each R^{Y} is independently selected from the group consisting of hydrogen, chloro, fluoro, CHF₂, CF₃, CH₃, CH₂CH₃, CH(CH₃)₂,

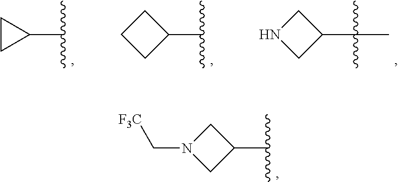

OCH₃, OCHF₂, OCF₃, OCH₂CF₃, OCH(CH₃)₂, and CN. In other embodiments, 2 R^{Y} on adjacent carbons, together with the atoms to which they are attached form a 1,3-dioxolanyl ring, which is optionally substituted with 1-2 R^{X}. In certain embodiments, each R^{X} is independently fluoro.

In further embodiments, R^{N4} is selected from the group consisting of hydrogen, phenyl (optionally substituted by one or more halo atoms), pyridyl (optionally substituted by CF₃), and cyclobutyl (optionally substituted by OCF₃).

In some embodiments, L² is a bond or C₁-C₆ alkylene, wherein C₁-C₆ alkylene is optionally substituted by 1-5 R^{L2}. In other embodiments, L² is a bond or C₁-C₆ alkylene, wherein C₁-C₆ alkylene is optionally substituted by 0 R^{L2}. For example, in certain embodiments L² is selected from a bond or —CH₂—. In further embodiments, L² is a bond.

In some embodiments, W is represented by Formula (W-a):

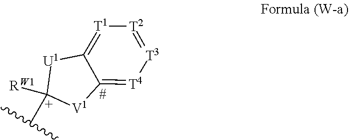

wherein:
T¹ is nitrogen or C(R^{W2});
T² is nitrogen or C(R^{W2});
T³ is nitrogen or C(R^{W2});
T⁴ is nitrogen or C(R^{W2});
wherein no more than two of T¹, T², T³, and T⁴ may be nitrogen;
U¹ is selected from the group consisting of a bond, —O—, —CO—, —NR^{N3}—, and —S(O)_{w}— (wherein w is 0, 1, or 2);
V¹ is selected from the group consisting of ⁺—O—^{#}, ⁺—C(R^{V11}R^{V12})—^{#}, ⁺—C(R^{V11}R^{V12})—C(O)—^{#}, ⁺—C(R^{V11}R^{V12})—C(═N—OH)—^{#}, ⁺—C(R^{V11}R^{V12})—C(R^{V13}R^{V14})—^{#}, ⁺—C(R^{V15}R^{V16})—O—^{#}, ⁺—C(R^{V15}R^{V16})—NR^{N3}—^{#}, ⁺—C(O)—NR^{N3}—^{#}, ⁺—NR^{N3}—^{#}, ⁺—O—C(R^{V15}R^{V16})—^{#}, ⁺—NR^{N3}—C(R^{V15}R^{V16})—^{#}, ⁺—NR^{N3}C(O)—^{#}, ⁺—C(O)—O—^{#}, ⁺—C(O)—^{#}, ⁺—C(R^{V15}R^{V16})—S(O)_{w}—^{#}, ⁺—S(O)_{w}—C(R^{V15}R^{V16})—^{#} (wherein w is 0, 1, or 2) and

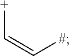

wherein the “⁺-” and “-^{#}” indicate the attachment points of V¹ as indicated in Formula (W-a);
wherein if V¹ is ⁺—O—^{#}, ⁺—NR^{N3}—^{#}, or ⁺—C(R^{V11}R^{V12})—^{#}; U¹ is not a bond;
R^{V11} and R^{V12} are each independently selected from the group consisting of hydrogen, C₁-C₆ alkyl, hydroxy-C₁-C₆ alkyl, halo-C₁-C₆ alkyl, amino-C₁-C₆ alkyl, cyano-C₁-C₆ alkyl, halo, cyano, —OR^{A}, —NR^{B}R^{C}, —NR^{B}R^{CC}, —NR^{B}C(O)R^{D}, —C(O)NR^{B}R^{C}, —C(O)R^{D}, —C(O)OH, —C(O)OR^{D}, —SR^{E}, —S(O)R^{D}, and —S(O)₂R^{D};
R^{V13} and R^{V14} are each independently selected from the group consisting of hydrogen, C₁-C₆ alkyl, hydroxy-C₁-C₆ alkyl, halo-C₁-C₆ alkyl, amino-C₁-C₆ alkyl, cyano-C₁-C₆ alkyl, halo, cyano, —OR^{A}, —NR^{B}R^{C}, —NR^{B}R^{CC}, —NR^{B}C(O)R^{D}, —C(O)NR^{B}R^{C}, —C(O)R^{D}, —C(O)OH, —C(O)OR^{D}, —SR^{E}, —S(O)R^{D}, and —S(O)₂R^{D};
R^{V15} and R^{V16} are each independently selected from the group consisting of hydrogen, C₁-C₆ alkyl, hydroxy-C₂-C₆ alkyl, halo-C₂-C₆ alkyl, amino-C₂-C₆ alkyl, cyano-C₂-C₆ alkyl, —C(O)NR^{B}R^{C}, —C(O)R^{D}, —C(O)OH, and —C(O)OR^{D}; and
R^{W1} is selected from the group consisting of hydrogen and C₁-C₆ alkyl.

In other embodiments, W is represented by Formula (W-a-1), Formula (W-a-2), Formula (W-a-3), Formula (W-a-4), or Formula (W-a-5):

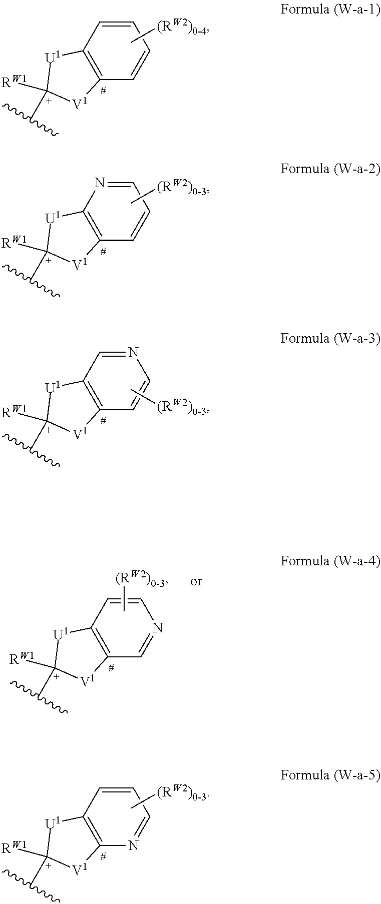

For example, in certain embodiments W is represented by Formula (W-a-1):

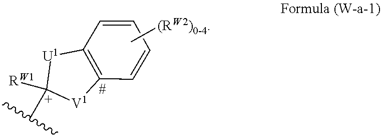

In some embodiments, U¹ is selected from the group consisting of a bond, —O—, —CO— and —NR^{N3}—; and V¹ is selected from the group consisting of ⁺—O—^{#}, ⁺—C(R^{V11}R^{V12})—^{#}, ⁺—C(R^{V11}R^{V12})—C(R^{V13}R^{V14})—^{#}, ⁺—C(R^{V15}R^{V16})—O—^{#}, ⁺—C(R^{V11}R^{V12})—C(O)—^{#}, ⁺—C(R^{V11}R^{V12})—C(═N—OH)—^{#}, ⁺—O—C(R^{V15}R^{V16})—^{#}, ⁺—C(R^{V15}R^{V16})—NR^{N3}—^{#}, ⁺—C(O)—NR^{N3}—^{#} and

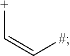

wherein “⁺-” and “-^{#}” indicate the attachment points of V¹ as indicated in Formula (W-a); and wherein if V¹ is ⁺—O—^{#} or ⁺—C(R^{V11}R^{V2})—^{#}, U¹ is not a bond.

In other embodiments, each of R^{V11}, R^{V12}, R^{V13}, and R^{V14} is independently selected from the group consisting of hydrogen, halo, C₁-C₃ alkyl, cyano, —OR^{A}, —NR^{B}R^{C} and —NR^{B}R^{CC}. For example, in certain embodiments each of R^{V11}, R^{V12}, R^{V13}, and R^{V14} is independently selected from the group consisting of hydrogen, hydroxyl, C₁-C₃ alkyl, —O—C₁-C₃ alkyl, —NR^{B}R^{C} and —NR^{B}R^{CC}.

In some embodiment, each of R^{V15} and R^{V16} is independently selected from the group consisting of hydrogen and C₁-C₃ alkyl. For example, in certain embodiments each of R^{V15} and R^{V16} is hydrogen.

In some embodiments, R^{V13} is selected from the group consisting of hydrogen, hydroxyl, CH₃, OCH₃, NH(CH₂)₂OH, NH(CH₂)₂CO₂H, NH(CH₂)₂CO₂CH₃, NH—SO₂—CH₃, NH(CO)CH₃, NH₂, NHCH₃, NHCH₂CF₃,

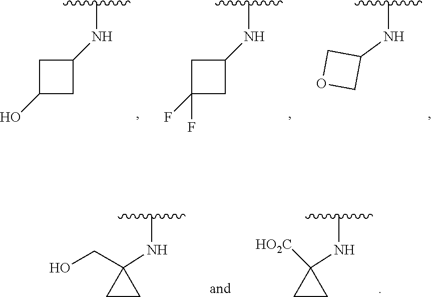 [SMILES: *NC1CC(O)C1 ; *NC1CC(F)(F)C1 ; *NC1COC1 ; *NC1(CO)CC1 ; *NC1(C(=O)O)CC1]

In other embodiments, R^{V12} is selected from the group consisting of hydrogen and C₁-C₃ alkyl.

In some embodiments, W is a benzo[d][1,3]dioxole, 3,4-dihydro-2H-benzo[b][1,4]oxazine, chromane, chromene, chroman-4-one, chroman-4-ol, chroman-4-one oxime, 2H-benzo[b][1,4]oxazin-3(4H)-one, 2,3-dihydrobenzo[b][1,4]dioxine, indoline, 2,3-dihydrobenzofuran or benzofuran-3(2H)-one moiety; wherein each of which is attached to L² through a saturated carbon atom, and wherein each of which is optionally substituted on one or more available unsaturated carbons with 1-4 R^{W2}, and wherein each R^{W2} is independently selected from the group consisting of C₁-C₆ alkyl, halo-C₁-C₆ alkyl, halo, oxo, cyano, and —OR^{A}.

For example, in some embodiments W is selected from the group consisting of:

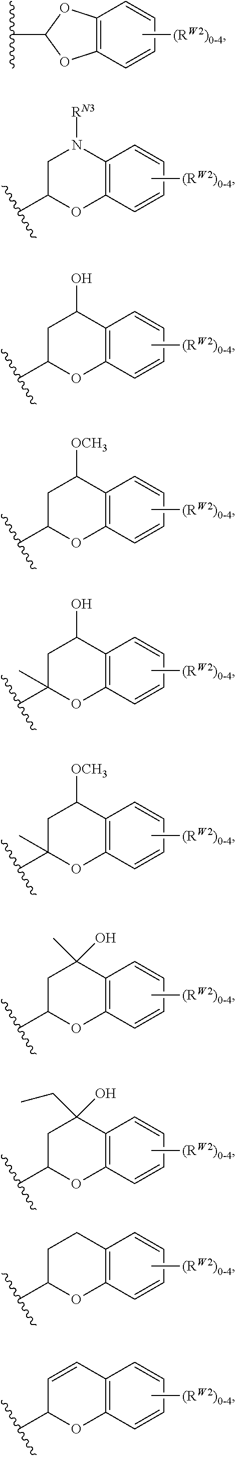

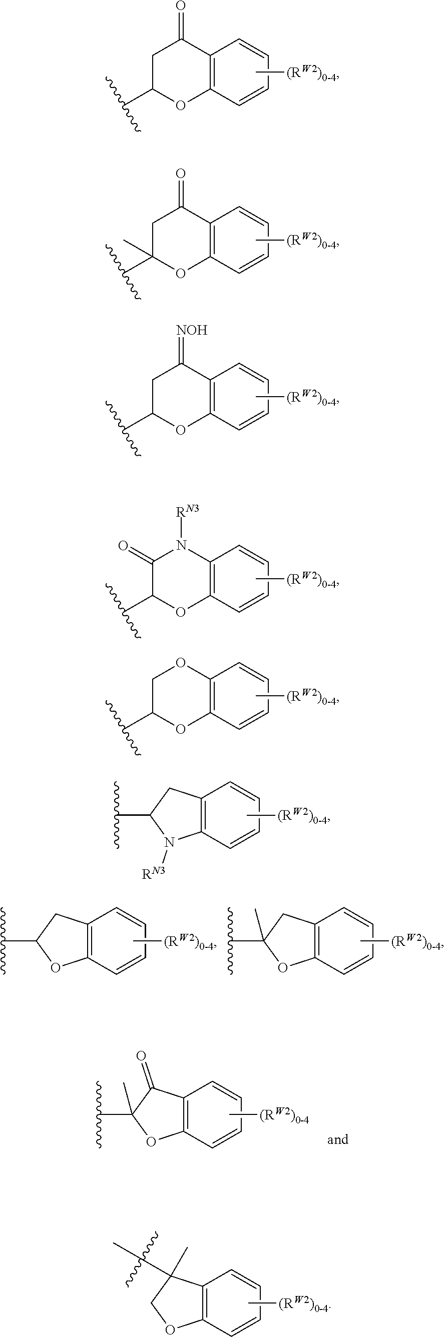

In other embodiments, W is represented by Formula (W-b):

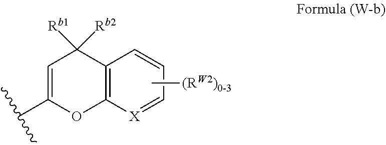

wherein:
X is nitrogen or C(R^{W2});
R^{b1} is hydrogen;
R^{b2} is hydroxyl; or
R^{b1} and R^{b2} taken together form an oxo moiety;
each R^{W2} is independently selected from the group consisting of hydrogen, C₁-C₆ alkyl, hydroxy-C₁-C₆ alkyl, hydroxy-C₂-C₆ alkyl-O—, halo-C₁-C₆ alkyl, halo-C₁-C₆ alkoxy, amino-C₁-C₆ alkyl, cyano-C₁-C₆ alkyl, halo, cyano, —OR^{A}, —NR^{B}R^{C}, —N^{B}C(O)R^{D}, —C(O)NR^{B}R^{C}, —C(O)R^{D}, —C(O)OH, —C(O)OR^{D}, —S(R^{F})ₘ, —S(O)R^{D}, and —S(O)₂R^{D}; or
2 R^{W2} groups on adjacent atoms, together with the atoms to which they are attached, form a 3-7-membered fused cycloalkyl, 3-7-membered fused heterocyclyl, fused aryl, or 5-6 membered fused heteroaryl, each of which is optionally substituted with 1-5 R^{X};
each R^{X} is independently selected from the group consisting of hydrogen, C₁-C₆ alkyl, hydroxy-C₁-C₆ alkyl, halo-C₁-C₆ alkyl, amino-C₁-C₆ alkyl, cyano-C₁-C₆ alkyl, oxo, halo, cyano, —OR^{A}, —NR^{B}R^{C}, —NR^{B}C(O)R^{D}, —C(O)NR^{B}R^{C}, —C(O)R^{D}, —C(O)OH, —C(O)OR^{D}, —SR^{E}, —S(O)R^{D}, and —S(O)₂R^{D};
R^{A} is, at each occurrence, independently hydrogen, C₁-C₆ alkyl, halo-C₁-C₆ alkyl, —C(O)NR^{B}R^{C}, —C(O)R^{D}, or —C(O)OR^{D};
each of R^{B} and R^{C} is independently hydrogen or C₁-C₆ alkyl;
R^{B} and R^{C} together with the atom to which they are attached form a 3-7-membered heterocyclyl ring optionally substituted with 1-3 R^{Z};
each R^{CC} is independently selected from the group consisting of hydroxy-C₁-C₆ alkyl, halo-C₁-C₆ alkyl, C₁-C₆ alkyl-CO₂H, C₁-C₆ alkyl-CO₂—C₁-C₆ alkyl, C(O) C₁-C₆ alkyl, S(O)₂—C₁-C₆ alkyl and 3-6-membered cycloalkyl; wherein 3-6-membered cycloalkyl may optionally be substituted by one or more substituents each independently selected from the group consisting of hydroxyl, halogen and —C(O)OH;
each R^{D} is independently C₁-C₆ alkyl or halo-C₁-C₆ alkyl;
each R^{E} is independently hydrogen, C₁-C₆ alkyl, or halo-C₁-C₆ alkyl;
each R^{F} is independently hydrogen, C₁-C₆ alkyl, or halo;
each R^{Z} is independently selected from the group consisting of C₁-C₆ alkyl, hydroxy-C₁-C₆ alkyl, halo-C₁-C₆ alkyl, halo, cyano, —OR^{A}, —NR^{B}R^{C}, —N^{B}C(O)R^{D}, —C(O)NR^{B}R^{C}, —C(O)R^{D}, —C(O)OH, —C(O)OR^{D}, and —S(O)₂R^{D}; and
m is 1 when R^{F} is hydrogen or C₁-C₆ alkyl, 3 when R^{F} is C₁-C₆ alkyl, or 5 when R^{F} is halo.

In some embodiments, X is C(R^{W2}). In other embodiments, R^{b1} and R^{b2} taken together form an oxo moiety. For example, in certain embodiments the compound is represented by:

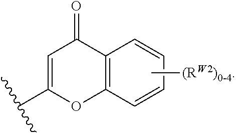

In other embodiments, R^{b1} is hydrogen and R^{b2} is hydroxyl. For example, in certain embodiments the compound is represented by:

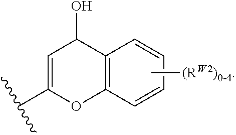

In some embodiments, R^{W2} is independently selected from the group consisting of C₁-C₆ alkyl, halo-C₁-C₆ alkyl, halo, cyano, and —OR^{A}, or 2 R^{W2} groups on adjacent carbons, together with the atoms to which they are attached form a 1,3-dioxolanyl ring, which is optionally substituted with 1-2 fluorine atoms.

In further embodiments, W is represented by Formula (W-c):

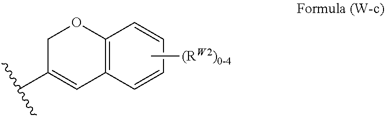

wherein:
each R^{W2} is independently selected from the group consisting of hydrogen, C₁-C₆ alkyl, hydroxy-C₁-C₆ alkyl, hydroxy-C₂-C₆ alkyl-O—, halo-C₁-C₆ alkyl, halo-C₁-C₆ alkoxy, amino-C₁-C₆ alkyl, cyano-C₁-C₆ alkyl, halo, cyano, —OR^{A}, —NR^{B}R^{C}, —N^{B}C(O)R^{D}, —C(O)NR^{B}R^{C}, —C(O)R^{D}, —C(O)OH, —C(O)OR^{D}, —S(R^{F})ₘ, —S(O)R^{D}, and —S(O)₂R^{D}; or
2 R^{W2} groups on adjacent atoms, together with the atoms to which they are attached, form a 3-7-membered fused cycloalkyl, 3-7-membered fused heterocyclyl, fused aryl, or 5-6 membered fused heteroaryl, each of which is optionally substituted with 1-5 R^{X};
each R^{X} is independently selected from the group consisting of hydrogen, C₁-C₆ alkyl, hydroxy-C₁-C₆ alkyl, halo-C₁-C₆ alkyl, amino-C₁-C₆ alkyl, cyano-C₁-C₆ alkyl, oxo, halo, cyano, —OR^{A}, —NR^{B}R^{C}, —N^{B}C(O)R^{D}, —C(O)NR^{B}R^{C}, —C(O)R^{D}, —C(O)OH, —C(O)OR^{D}, —SR^{E}, —S(O)R^{D}, and —S(O)₂R^{D};
R^{A} is, at each occurrence, independently hydrogen, C₁-C₆ alkyl, halo-C₁-C₆ alkyl, —C(O)NR^{B}R^{C}, —C(O)R^{D}, or —C(O)OR^{D};
each of R^{B} and R^{C} is independently hydrogen or C₁-C₆ alkyl;
R^{B} and R^{C} together with the atom to which they are attached form a 3-7-membered heterocyclyl ring optionally substituted with 1-3 R^{Z};
each R^{CC} is independently selected from the group consisting of hydroxy-C₁-C₆ alkyl, halo-C₁-C₆ alkyl, C₁-C₆ alkyl-CO₂H, C₁-C₆ alkyl-CO₂—C₁-C₆ alkyl, C(O) C₁-C₆ alkyl, S(O)₂—C₁-C₆ alkyl and 3-6-membered cycloalkyl; wherein 3-6-membered cycloalkyl may optionally be substituted by one or more substituents each independently selected from the group consisting of hydroxyl, halogen and —C(O)OH;
each R^{D} is independently C₁-C₆ alkyl or halo-C₁-C₆ alkyl;
each R^{E} is independently hydrogen, C₁-C₆ alkyl, or halo-C₁-C₆ alkyl;
each R^{F} is independently hydrogen, C₁-C₆ alkyl, or halo;
each R^{Z} is independently selected from the group consisting of C₁-C₆ alkyl, hydroxy-C₁-C₆ alkyl, halo-C₁-C₆ alkyl, halo, cyano, —OR^{A}, —NR^{B}R^{C}, —NR^{B}C(O)R^{D}, —C(O)NR^{B}R^{C}, —C(O)R^{D}, —C(O)OH, —C(O)OR^{D}, and —S(O)₂R^{D}; and
m is 1 when R^{F} is hydrogen or C₁-C₆ alkyl, 3 when R^{F} is C₁-C₆ alkyl, or 5 when R^{F} is halo.

In some embodiments, R^{W2} is independently selected from the group consisting of C₁-C₆ alkyl, halo-C₁-C₆ alkyl, halo, cyano, and —OR^{A}, or 2 R^{W2} groups on adjacent carbons, together with the atoms to which they are attached form a 1,3-dioxolanyl ring, which is optionally substituted with 1-2 fluorine atoms.

In yet further embodiments, W is represented by Formula (W-d):

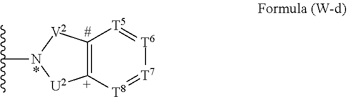

wherein:
T⁵ is nitrogen or C(R^{W2});
T⁶ is nitrogen or C(R^{W2});
T⁷ is nitrogen or C(R^{W2});
T⁸ is nitrogen or C(R^{W2});
wherein no more than two of T⁵, T⁶, T⁷, and T⁸ may be nitrogen;
V² is selected from the group consisting of ^{*}—C(R^{V21}R^{V22})—^{#}, ^{*}—C(R^{V21}R^{V22})—C(R^{V23}R^{V24})—^{#}, ^{*}—C(R^{V21}R^{V22})—C(R^{V23}R^{V24})—C(R^{V23}R^{V24})—^{#}, ^{*}—C(R^{V21}R^{V22})—C(R^{V21}R^{V22})—O—^{#}, ^{*}—C(R^{V21}R^{V22})—C(R^{V21}R^{V22})—NR^{N3}—^{#}, —C(R^{V21}R^{V22})—NR^{N3}—^{#}, ^{*}—C(O)—C(R^{V23}R^{V24})—^{#}, ^{*}—C(O)—C(R^{V23}R^{V24})—C(R^{V23}R^{V24})—^{#}, ^{*}—C(O)—NR^{N3}—^{#} and ^{*}—C(O)—O—^{#}, wherein “^{*}-” and “-^{#}” indicate the attachment points of V² as indicated in Formula (W-d);
U² is selected from the group consisting of a bond, ^{*}—C(O)—⁺, and ^{*}—C(R^{U21}R^{U22})—⁺, wherein “^{*}-” and “-⁺” indicate the attachment points of U² as indicated in Formula (W-d);
wherein if V² is ^{*}—C(R^{V21}R^{V22})—^{#}, U² is not a bond;
R^{U21} and R^{U22} are each independently selected from the group consisting of hydrogen, C₁-C₆ alkyl, hydroxy-C₂-C₆ alkyl, halo-C₂-C₆ alkyl, amino-C₂-C₆ alkyl, cyano-C₂-C₆ alkyl, —C(O)NR^{B}R^{C}, —C(O)R^{D}, —C(O)OH, —C(O)OR^{D}, C₁-C₆ alkyl-C(O)OH, and C₁-C₆ alkyl-C(O)OR^{D};
R^{V21} and R^{V22} are each independently selected from the group consisting of hydrogen, C₁-C₆ alkyl, hydroxy-C₂-C₆ alkyl, halo-C₂-C₆ alkyl, amino-C₂-C₆ alkyl, cyano-C₂-C₆ alkyl, —C(O)NR^{B}R^{C}, —C(O)R^{D}, —C(O)OH, and —C(O)OR^{D}; and
R^{V23} and R^{V24} are each independently selected from the group consisting of hydrogen, C₁-C₆ alkyl, hydroxy-C₁-C₆ alkyl, halo-C₁-C₆ alkyl, amino-C₁-C₆ alkyl, cyano-C₁-C₆ alkyl, halo, cyano, —OR^{A}, —NR^{B}R^{C}, —NR^{B}C(O)R^{D}, —C(O)NR^{B}R^{C}, —C(O)R^{D}, —C(O)OH, —C(O)OR^{D}, —SR^{E}, —S(O)R^{D}, and —S(O)₂R^{D}.

For example, in some embodiments W is represented by Formula (W-d-1), Formula (W-d-2), Formula (W-d-3), Formula (W-d-4), or Formula (W-d-5):

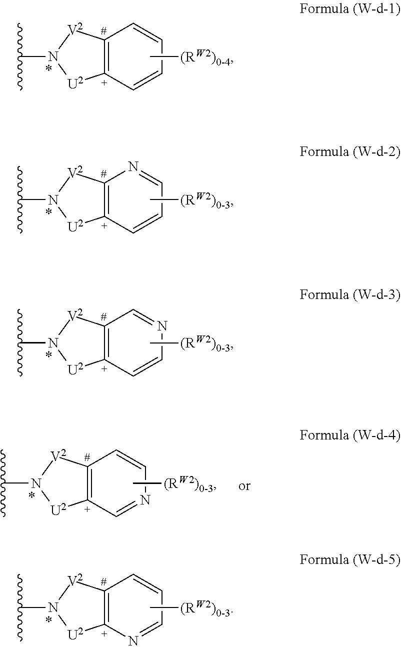

In certain embodiment, W is represented by Formula (W-d-1):

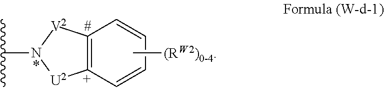

In some embodiments, V² is selected from the group consisting of ^{*}—C(R^{V21}R^{V22})—^{#}, ^{*}—C(R^{V21}R^{V22})—C(R^{V23}R^{V24})—^{#}, ^{*}—C(O)—C(R^{V23}R^{V24})—^{#}, and ^{*}—C(R^{V21}R^{V22})—C(R^{V23}R^{V24})—C(R^{V23}R^{V24})—^{#}; wherein “^{*}-” and “-^{#}” indicate the attachment points of V² as indicated in Formula (W-d). For example, in some embodiments each of R^{V21} and R^{V22} is independently selected from the group consisting of hydrogen and C₁-C₃ alkyl. In certain embodiments, each of R^{V21} and R^{V22} is hydrogen. In other embodiments, each of R^{V23} and R^{V24} is independently selected from the group consisting of hydrogen, halo, C₁-C₃ alkyl, cyano, —OR^{A}, and —NR^{B}R^{C}. For example, in certain embodiments each of R^{V23} and R^{V24} is hydrogen.

In some embodiments, U² is selected from the group consisting of a bond, ^{*}—C(O)—⁺, ^{*}—CH₂—⁺, and ^{*}—CH(CH₂CO₂H)—⁺, wherein “^{*}-” and “-⁺” indicate the attachment points of U² as indicated in Formula (W-d); and V² is selected from the group consisting of ^{*}—CH₂—^{#}, ^{*}—CH₂—CH₂—^{#}, ^{*}—C(O)—CH₂—^{#}, ^{*}—C(O)—NH—^{#}, ^{*}—CH₂—NH—^{#}, and ^{*}—CH₂—CH₂—CH₂—^{#}; wherein “^{*}-” and “-^{#}” indicate the attachment points of V² as indicated in Formula (W-d).

In some embodiments, W is an indoline, indolin-2-one, isoindoline, isoindolin-1-one, 1,2,3,4-tetrahydroquinoline, 1,2,3,4-tetrahydroisoquinoline, quinazoline-2,4(1H,3H)-dione, or 2,3-dihydroquinazolin-4(1H)-one moiety; wherein each of which is attached to L² through a nitrogen atom, and wherein each of which is optionally substituted on one or more available unsaturated carbon atoms with 1-4 R^{W2}, and wherein each R^{W2} is independently selected from the group consisting of C₁-C₆ alkyl, halo-C₁-C₆ alkyl, hydroxy-C₂-C₆ alkyl-O—, halo, cyano, and —OR^{A}. For example, in certain embodiments W is selected from the group consisting of:

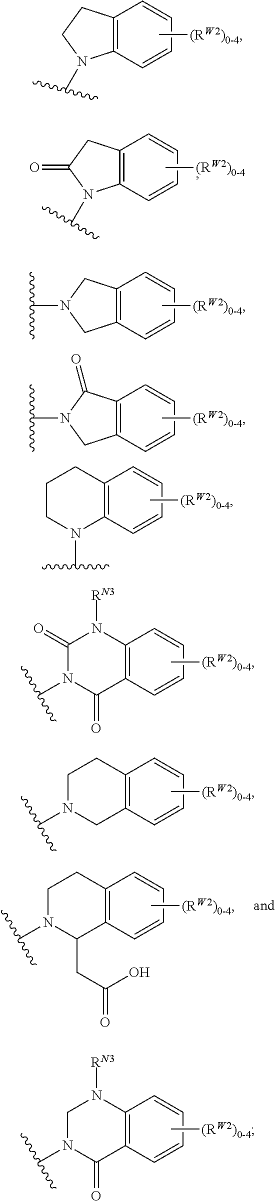

wherein R^{N3} is selected from the group consisting of hydrogen, C₁-C₆ alkyl, and hydroxy-C₂-C₆ alkyl.

In some embodiments, the compound of Formula (I) is a compound of Formula (I-a):

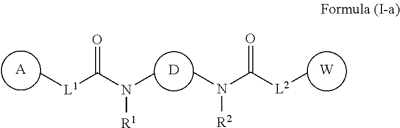

or a pharmaceutically acceptable salt, solvate, hydrate, tautomer, N-oxide, or stereoisomer thereof, wherein:
D is bicyclo[1.1.1]pentanyl or bicyclo[2.2.2]octanyl, each of which is optionally substituted with 1-4 R^{X} groups;
L¹ is selected from the group consisting of a bond and CH₂O—*, wherein “-*” indicates the attachment point to A;
L² is a bond;
R¹ is selected from the group consisting of hydrogen and CH₃;
R² is selected from the group consisting of hydrogen and CH₃;
A is phenyl, pyrazinyl or pyridyl, each of which is optionally substituted with 1-5 R^{Y} groups;
W is a benzo[d][1,3]dioxole, 3,4-dihydro-2H-benzo[b][1,4]oxazine, chromane, chromene, chroman-4-one, chroman-4-ol, chroman-4-one oxime, 2H-benzo[b][1,4]oxazin-3(4H)-one, 2,3-dihydrobenzo[b][1,4]dioxine, indoline, 2,3-dihydrobenzofuran, benzofuran-3(2H)-one, 4H-chromen-4-ol or 4H-chromen-4-one moiety; wherein each of which is attached to L² through a carbon atom, and wherein each of which is optionally substituted on one or more available aromatic carbon atoms with 1-4 R^{W2} groups; and wherein 3,4-dihydro-2H-benzo[b][1,4]oxazine, 2H-benzo[b][1,4]oxazin-3(4H)-one, and indoline may be optionally substituted on an available nitrogen atom with hydrogen or CH₃;
each R^{W2} is independently selected from the group consisting of hydrogen, chloro, fluoro, CHF₂, CF₃, CH₃, CH₂CH₃, CH(CH₃)₂, OCH₃, OCHF₂, OCF₃, OCH₂CF₃, OCH(CH₃)₂, and CN; or
2 R^{W2} groups on adjacent carbons, together with the atoms to which they are attached form a 1,3-dioxolanyl ring, which is optionally substituted with 1-2 fluorine atoms;
each R^{X} is independently fluoro, oxo, OH, OCH₃, C(O)OH, or C(O)OCH₃; and
each R^{Y} is independently chloro, fluoro, CHF₂, CF₃, CH₃, CH₂CH₃, CH(CH₃)₂, OCH₃, OCHF₂, OCF₃, OCH₂CF₃, OCH(CH₃)₂, or CN; or
2 R^{Y} groups on adjacent atoms, together with the atoms to which they are attached form a 1,3-dioxolanyl ring, which is optionally substituted with 1-2 fluorine atoms.

In some embodiments, the compound of Formula (I) is a compound of Formula (I-b):

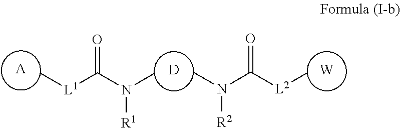

or a pharmaceutically acceptable salt, solvate, hydrate, tautomer, N-oxide, or stereoisomer thereof, wherein:
D is bicyclo[1.1.1]pentanyl or bicyclo[2.2.2]octanyl, each of which is optionally substituted with 1-4 R^{X} groups;
L¹ is selected from the group consisting of a bond and CH₂O—*, wherein “-*” indicates the attachment point to A;
L² is CH₂—*, wherein “-*” indicates the attachment point to W;
R¹ is selected from the group consisting of hydrogen and CH₃;
R² is selected from the group consisting of hydrogen and CH₃;
A is phenyl, pyrazinyl or pyridyl, each of which is optionally substituted with 1-5 R^{Y} groups;
W is an indoline or tetrahydroisoquinoline moiety; wherein indoline or tetrahydroisoquinoline is attached to L² through a nitrogen atom, and wherein indoline or tetrahydroisoquinoline is optionally substituted on one or more available unsaturated carbon atoms with 1-4 R^{W2} groups;
each R^{W2} is independently selected from the group consisting of hydrogen, chloro, fluoro, CHF₂, CF₃, CH₃, CH₂CH₃, CH(CH₃)₂, OCH₃, OCHF₂, OCF₃, OCH₂CF₃, OCH(CH₃)₂, and CN; or
2 R^{W2} groups on adjacent carbons, together with the atoms to which they are attached form a 1,3-dioxolanyl ring, which is optionally substituted with 1-2 fluorine atoms;
each R^{X} is independently fluoro, oxo, OH, OCH₃, C(O)OH, or C(O)OCH₃; and
each R^{Y} is independently chloro, fluoro, CHF₂, CF₃, CH₃, CH₂CH₃, CH(CH₃)₂, OCH₃, OCHF₂, OCF₃, OCH₂CF₃, OCH(CH₃)₂, or CN; or
2 R^{Y} groups on adjacent atoms, together with the atoms to which they are attached form a 1,3-dioxolanyl ring, which is optionally substituted with 1-2 fluorine atoms.

In some embodiments, the compound of Formula (I) is a compound of Formula (I-e-1), Formula (I-e-2), Formula (I-e-3), Formula (I-e-4), Formula (I-e-5), Formula (I-e-6), Formula (I-e-7), Formula (I-e-8), Formula (I-e-9), Formula (I-e-10), Formula (I-e-11), Formula (I-e-12), Formula (I-e-13), Formula (I-e-14), Formula (I-e-15), Formula (I-e-16), or Formula (I-e-17):

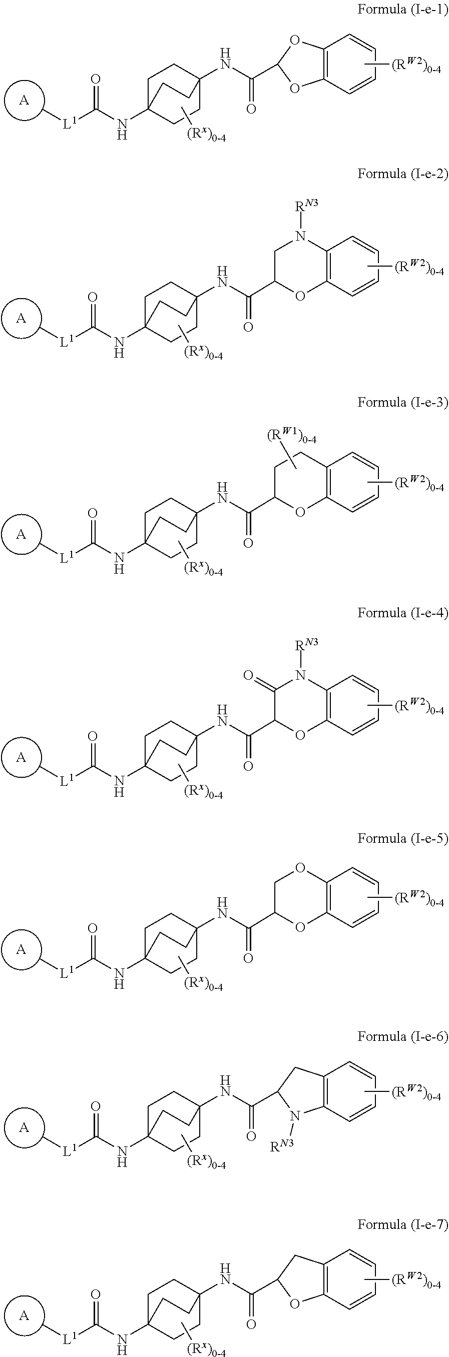

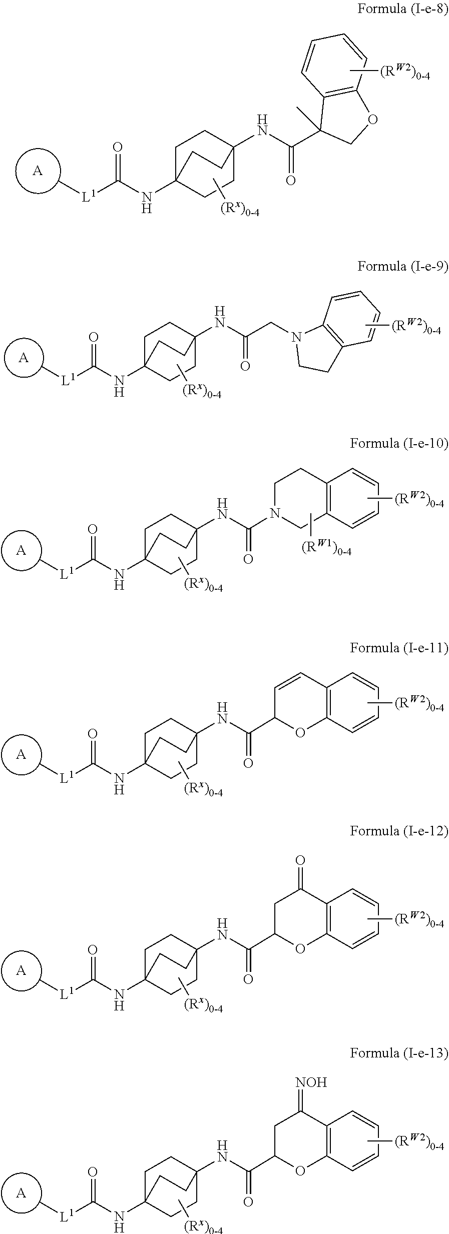

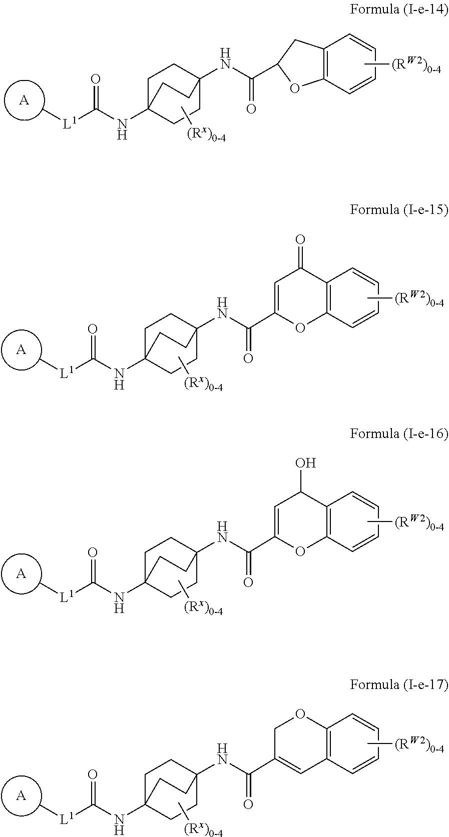

or a pharmaceutically acceptable salt, solvate, hydrate, tautomer, N-oxide, or stereoisomer thereof, wherein each of A, L¹, R^{N3}, R^{W1} R^{W2} and R^{X} is defined as for Formula (I).

In some embodiments, the compound of Formula (I) is a compound of Formula (I-f-1), Formula (I-f-2), Formula (I-f-3), Formula (I-f-4), Formula (I-f-5), Formula (I-f-6), Formula (I-f-7), Formula (I-f-8), Formula (I-f-9), Formula (I-f-10), Formula (I-f-11), Formula (I-f-12), Formula (I-f-13), Formula (I-f-14), Formula (I-f-15), Formula (I-f-16), or Formula (I-f-17):

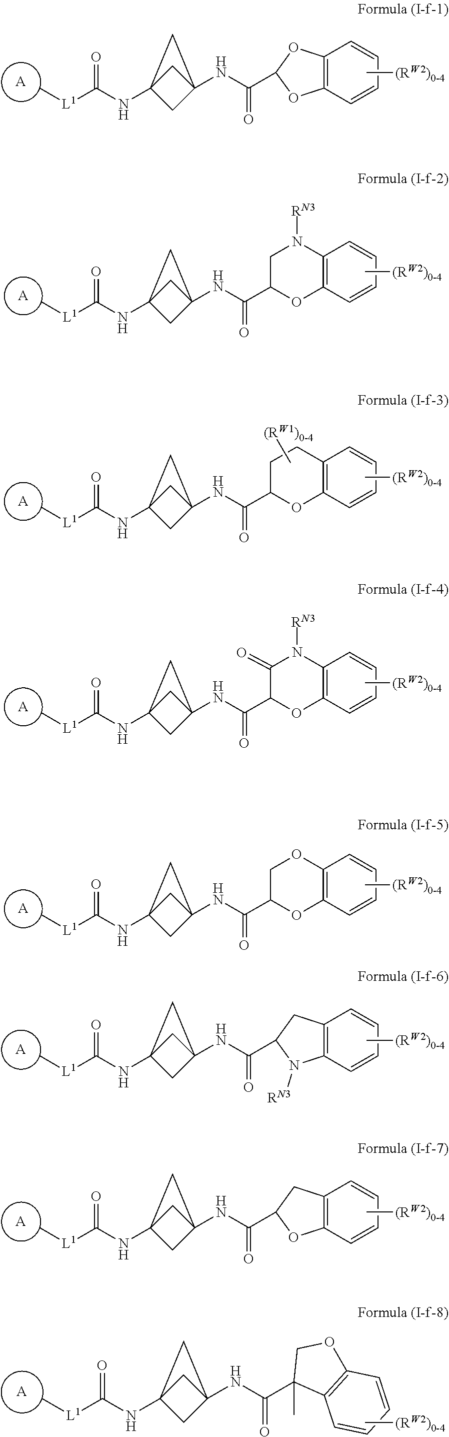

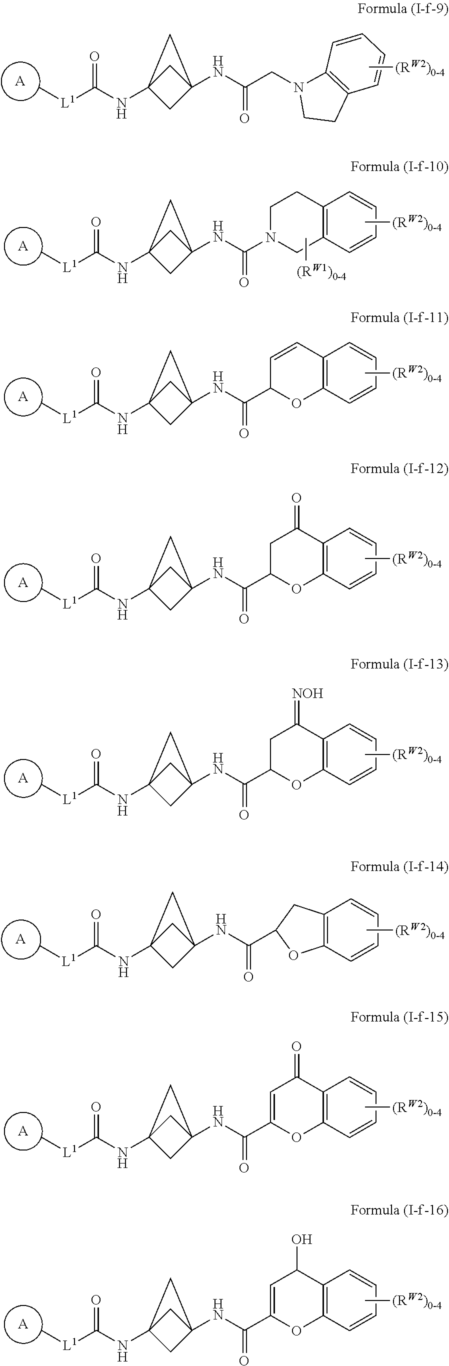

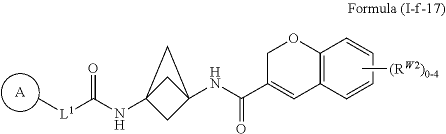

or a pharmaceutically acceptable salt, solvate, hydrate, tautomer, N-oxide, or stereoisomer thereof, wherein each of A, L¹, R^{N3}, R^{W1} and R^{W2} is defined as for Formula (I).

In some embodiments, a disclosed compound is selected from the group consisting of:
N-{4-[2-(4-chloro-3-fluorophenoxy)acetamido]bicyclo[2.2.2]octan-1-yl}-2H-1,3-benzodioxole-2-carboxamide;
2-(5-chloro-2,3-dihydro-1H-indol-1-yl)-N-{(3S)-4-[2-(4-chloro-3-fluorophenoxy)acetamido]-3-hydroxybicyclo[2.2.2]octan-1-yl}acetamide;
(2R)-6-chloro-N-{3-[2-(4-chloro-3-fluorophenoxy)acetamido]bicyclo[1.1.1]pentan-1-yl}-3,4-dihydro-2H-1,4-benzoxazine-2-carboxamide;
(2S)-6-chloro-N-{3-[2-(4-chloro-3-fluorophenoxy)acetamido]bicyclo[1.1.1]pentan-1-yl}-3,4-dihydro-2H-1,4-benzoxazine-2-carboxamide;
(2S)-6-chloro-N-{3-[2-(4-chloro-3-fluorophenoxy)acetamido]bicyclo[1.1.1]pentan-1-yl}-4-methyl-3,4-dihydro-2H-1,4-benzoxazine-2-carboxamide;
(2S)—N-{(3S)-4-[2-(4-chloro-3-fluorophenoxy)acetamido]-3-hydroxybicyclo[2.2.2]octan-1-yl}-6-fluoro-3,4-dihydro-2H-1-benzopyran-2-carboxamide;
(2R)—N-{(3S)-4-[2-(4-chloro-3-fluorophenoxy)acetamido]-3-hydroxybicyclo[2.2.2]octan-1-yl}-6-fluoro-3,4-dihydro-2H-1-benzopyran-2-carboxamide;
6-chloro-N-{(3S)-4-[2-(4-chloro-3-fluorophenoxy)acetamido]-3-hydroxybicyclo[2.2.2]octan-1-yl}-4-methyl-3,4-dihydro-2H-1,4-benzoxazine-2-carboxamide;
N-{3-[2-(4-chloro-3-fluorophenoxy)acetamido]bicyclo[1.1.1]pentan-1-yl}-6,7-difluoro-3,4-dihydro-2H-1,4-benzoxazine-2-carboxamide;
N-{3-[2-(4-chloro-3-fluorophenoxy)acetamido]bicyclo[1.1.1]pentan-1-yl}-3-oxo-3,4-dihydro-2H-1,4-benzoxazine-2-carboxamide;
N-{(3S)-4-[2-(4-chloro-3-fluorophenoxy)acetamido]-3-hydroxybicyclo[2.2.2]octan-1-yl}-3,4-dihydro-2H-1,4-benzoxazine-2-carboxamide;
N-{(3S)-4-[2-(4-chloro-3-fluorophenoxy)acetamido]-3-hydroxybicyclo[2.2.2]octan-1-yl}-4-methyl-3,4-dihydro-2H-1,4-benzoxazine-2-carboxamide;
6-chloro-N-{3-[2-(4-chloro-3-fluorophenoxy)acetamido]bicyclo[1.1.1]pentan-1-yl}-4-methyl-3,4-dihydro-2H-1,4-benzoxazine-2-carboxamide;
6-chloro-N-{3-[2-(4-chloro-3-fluorophenoxy)acetamido]bicyclo[1.1.1]pentan-1-yl}-3,4-dihydro-2H-1,4-benzoxazine-2-carboxamide;
6-chloro-N-{(3S)-4-[2-(4-chloro-3-fluorophenoxy)acetamido]-3-hydroxybicyclo[2.2.2]octan-1-yl}-3,4-dihydro-2H-1,4-benzoxazine-2-carboxamide;
(2R)-6-chloro-N-{(3S)-4-[2-(4-chloro-3-fluorophenoxy)acetamido]-3-hydroxybicyclo[2.2.2]octan-1-yl}-2,3-dihydro-1,4-benzodioxine-2-carboxamide;
(2R)-6-chloro-N-{(3S)-4-[2-(3,4-dichlorophenoxy)acetamido]-3-hydroxybicyclo[2.2.2]octan-1-yl}-2,3-dihydro-1,4-benzodioxine-2-carboxamide;
6,7-dichloro-N-{3-[2-(3,4-difluorophenoxy)acetamido]bicyclo[1.1.1]pentan-1-yl}-2,3-dihydro-1,4-benzodioxine-2-carboxamide;
N-{3-[2-(4-chloro-3-fluorophenoxy)acetamido]bicyclo[1.1.1]pentan-1-yl}-6,7-difluoro-2,3-dihydro-1,4-benzodioxine-2-carboxamide;
(2R)-6-chloro-N-{3-[2-(4-chloro-3-fluorophenoxy)acetamido]bicyclo[1.1.1]pentan-1-yl}-2,3-dihydro-1,4-benzodioxine-2-carboxamide;
(2R)—N-{3-[2-(4-chloro-3-fluorophenoxy)acetamido]bicyclo[1.1.1]pentan-1-yl}-6-fluoro-3,4-dihydro-2H-1-benzopyran-2-carboxamide;
(2S)—N-{3-[2-(4-chloro-3-fluorophenoxy)acetamido]bicyclo[1.1.1]pentan-1-yl}-6-fluoro-3,4-dihydro-2H-1-benzopyran-2-carboxamide;
N-{3-[2-(4-chloro-3-fluorophenoxy)acetamido]bicyclo[1.1.1]pentan-1-yl}-2,3-dihydro-1,4-benzodioxine-2-carboxamide;
N-{(2R)-4-[2-(4-chloro-3-fluorophenoxy)acetamido]-2-hydroxybicyclo[2.2.2]octan-1-yl}-2,3-dihydro-1,4-benzodioxine-2-carboxamide;
(2R)—N-{3-[2-(4-chloro-3-fluorophenoxy)acetamido]bicyclo[1.1.1]pentan-1-yl}-2,3-dihydro-1,4-benzodioxine-2-carboxamide;
(2S)—N-{3-[2-(4-chloro-3-fluorophenoxy)acetamido]bicyclo[1.1.1]pentan-1-yl}-2,3-dihydro-1,4-benzodioxine-2-carboxamide;
(2R)—N-{(2S)-4-[2-(4-chloro-3-fluorophenoxy)acetamido]-2-hydroxybicyclo[2.2.2]octan-1-yl}-2,3-dihydro-1,4-benzodioxine-2-carboxamide;
(2S)—N-{(2S)-4-[2-(4-chloro-3-fluorophenoxy)acetamido]-2-hydroxybicyclo[2.2.2]octan-1-yl}-2,3-dihydro-1,4-benzodioxine-2-carboxamide;
(2R)—N-{3-[2-(4-chloro-3-fluorophenoxy)acetamido]bicyclo[1.1.1]pentan-1-yl}-2,3-dihydro-1H-indole-2-carboxamide;
N-{4-[2-(4-chloro-3-fluorophenoxy)acetamido]-2-hydroxybicyclo[2.2.2]octan-1-yl}-2,3-dihydro-1-benzofuran-2-carboxamide;
(7S)—N-{3-[2-(4-chloro-3-fluorophenoxy)acetamido]bicyclo[1.1.1]pentan-1-yl}-2,2-difluoro-7-methyl-6,7-dihydro-2H-furo[2,3-f][1,3]benzodioxole-7-carboxamide;
(7R)—N-{3-[2-(4-chloro-3-fluorophenoxy)acetamido]bicyclo[1.1.1]pentan-1-yl}-2,2-difluoro-7-methyl-6,7-dihydro-2H-furo[2,3-f][1,3]benzodioxole-7-carboxamide;
N-{3-[(6,7-dichloro-2,3-dihydro-1,4-benzodioxine-2-carbonyl)amino]bicyclo[1.1.1]pentan-1-yl}-5-(trifluoromethoxy)pyridine-2-carboxamide;
N-{3-[(6,7-dichloro-2,3-dihydro-1,4-benzodioxine-2-carbonyl)amino]bicyclo[1.1.1]pentan-1-yl}-5-(difluoromethyl)pyrazine-2-carboxamide;
(2R)-6-chloro-N-{3-[2-(4-chloro-3-fluorophenoxy)acetamido]bicyclo[1.1.1]pentan-1-yl}-4-methyl-3,4-dihydro-2H-1,4-benzoxazine-2-carboxamide;
6-chloro-N-{3-[2-(4-chloro-3-fluorophenoxy)acetamido]bicyclo[1.1.1]pentan-1-yl}-4-(2-hydroxyethyl)-3,4-dihydro-2H-1,4-benzoxazine-2-carboxamide;
6-chloro-N-{3-[2-(4-chloro-3-fluorophenoxy)acetamido]bicyclo[1.1.1]pentan-1-yl}-4-(hydroxyacetyl)-3,4-dihydro-2H-1,4-benzoxazine-2-carboxamide;
6-chloro-N-{3-[2-(4-chloro-3-fluorophenoxy)acetamido]bicyclo[1.1.1]pentan-1-yl}-4-(1,2-dimethyl-1H-imidazole-5-sulfonyl)-3,4-dihydro-2H-1,4-benzoxazine-2-carboxamide;
6-chloro-N-{3-[2-(4-chloro-3-fluorophenoxy)acetamido]bicyclo[1.1.1]pentan-1-yl}-4-[(1R,2S)-2-fluorocyclopropane-1-carbonyl]-3,4-dihydro-2H-1,4-benzoxazine-2-carboxamide;
6-chloro-N-{3-[2-(4-chloro-3-fluorophenoxy)acetamido]bicyclo[1.1.1]pentan-1-yl}-4-(2-ethoxyethanesulfonyl)-3,4-dihydro-2H-1,4-benzoxazine-2-carboxamide;
6-chloro-N-{3-[2-(4-chloro-3-fluorophenoxy)acetamido]bicyclo[1.1.1]pentan-1-yl}-4-(1-fluorocyclopropane-1-carbonyl)-3,4-dihydro-2H-1,4-benzoxazine-2-carboxamide;
6-chloro-N-{3-[2-(4-chloro-3-fluorophenoxy)acetamido]bicyclo[1.1.1]pentan-1-yl}-4-(3,3,4,4,4-pentafluorobutanoyl)-3,4-dihydro-2H-1,4-benzoxazine-2-carboxamide;
rac-(2R)-6-chloro-N-{3-[2-(4-chloro-3-fluorophenoxy)acetamido]bicyclo[1.1.1]pentan-1-yl}-4-[(1R,2R)-2-fluorocyclopropane-1-carbonyl]-3,4-dihydro-2H-1,4-benzoxazine-2-carboxamide;
6-chloro-N-{3-[2-(4-chloro-3-fluorophenoxy)acetamido]bicyclo[1.1.1]pentan-1-yl}-4-[4-(trifluoromethoxy)benzoyl]-3,4-dihydro-2H-1,4-benzoxazine-2-carboxamide;
rac-(2R)-6-chloro-N-{3-[2-(4-chloro-3-fluorophenoxy)acetamido]bicyclo[1.1.1]pentan-1-yl}-4-[(1S,2S)-2-fluorocyclopropane-1-carbonyl]-3,4-dihydro-2H-1,4-benzoxazine-2-carboxamide;
6-chloro-N-{3-[2-(4-chloro-3-fluorophenoxy)acetamido]bicyclo[1.1.1]pentan-1-yl}-4-(5-methylfuran-2-carbonyl)-3,4-dihydro-2H-1,4-benzoxazine-2-carboxamide;
6-chloro-N-{3-[2-(4-chloro-3-fluorophenoxy)acetamido]bicyclo[1.1.1]pentan-1-yl}-4-(3-methoxybenzoyl)-3,4-dihydro-2H-1,4-benzoxazine-2-carboxamide;
4-[6-chloro-2-({3-[2-(4-chloro-3-fluorophenoxy)acetamido]bicyclo[1.1.1]pentan-1-yl}carbamoyl)-2,3-dihydro-4H-1,4-benzoxazine-4-carbonyl]benzene-1-sulfonyl fluoride; 6-chloro-N-{3-[2-(4-chloro-3-fluorophenoxy)acetamido]bicyclo[1.1.1]pentan-1-yl}-4-(4,4,4-trifluorobutanoyl)-3,4-dihydro-2H-1,4-benzoxazine-2-carboxamide;
6-chloro-N-{3-[2-(4-chloro-3-fluorophenoxy)acetamido]bicyclo[1.1.1]pentan-1-yl}-4-(cyclopropanecarbonyl)-3,4-dihydro-2H-1,4-benzoxazine-2-carboxamide;
6-chloro-N-{3-[2-(4-chloro-3-fluorophenoxy)acetamido]bicyclo[1.1.1]pentan-1-yl}-4-(4-methoxybenzoyl)-3,4-dihydro-2H-1,4-benzoxazine-2-carboxamide;
6-chloro-N-{3-[2-(4-chloro-3-fluorophenoxy)acetamido]bicyclo[1.1.1]pentan-1-yl}-4-(oxane-4-carbonyl)-3,4-dihydro-2H-1,4-benzoxazine-2-carboxamide;
6-chloro-N-{3-[2-(4-chloro-3-fluorophenoxy)acetamido]bicyclo[1.1.1]pentan-1-yl}-4-(oxolane-3-carbonyl)-3,4-dihydro-2H-1,4-benzoxazine-2-carboxamide;
6-chloro-N-{3-[2-(4-chloro-3-fluorophenoxy)acetamido]bicyclo[1.1.1]pentan-1-yl}-4-(5-methyl-1,2-oxazole-4-carbonyl)-3,4-dihydro-2H-1,4-benzoxazine-2-carboxamide;
6-chloro-N-{3-[2-(4-chloro-3-fluorophenoxy)acetamido]bicyclo[1.1.1]pentan-1-yl}-4-(1,2-oxazole-5-carbonyl)-3,4-dihydro-2H-1,4-benzoxazine-2-carboxamide;
[2-({3-[2-(4-chloro-3-fluorophenoxy)acetamido]bicyclo[1.1.1]pentan-1-yl}carbamoyl)-1,2,3,4-tetrahydroisoquinolin-1-yl]acetic acid;
6-chloro-N-{3-[2-(4-chloro-3-fluorophenoxy)acetamido]bicyclo[1.1.1]pentan-1-yl}-4-[(2-methoxyethoxy)acetyl]-3,4-dihydro-2H-1,4-benzoxazine-2-carboxamide;
4-acetyl-6-chloro-N-{3-[2-(4-chloro-3-fluorophenoxy)acetamido]bicyclo[1.1.1]pentan-1-yl}-3,4-dihydro-2H-1,4-benzoxazine-2-carboxamide;
6-chloro-N-{3-[2-(4-chloro-3-fluorophenoxy)acetamido]bicyclo[1.1.1]pentan-1-yl}-4-(methoxyacetyl)-3,4-dihydro-2H-1,4-benzoxazine-2-carboxamide;
6-chloro-N-{3-[2-(4-chloro-3-fluorophenoxy)acetamido]bicyclo[1.1.1]pentan-1-yl}-4-(2,2,3,3,4,4,4-heptafluorobutanoyl)-3,4-dihydro-2H-1,4-benzoxazine-2-carboxamide;
6-chloro-N-{3-[2-(4-chloro-3-fluorophenoxy)acetamido]bicyclo[1.1.1]pentan-1-yl}-4-[3-(trifluoromethyl)benzoyl]-3,4-dihydro-2H-1,4-benzoxazine-2-carboxamide;
6-chloro-N-{3-[2-(4-chloro-3-fluorophenoxy)acetamido]bicyclo[1.1.1]pentan-1-yl}-4-(2,2,3,3-tetrafluoropropanoyl)-3,4-dihydro-2H-1,4-benzoxazine-2-carboxamide;
6-chloro-N-{3-[2-(4-chloro-3-fluorophenoxy)acetamido]bicyclo[1.1.1]pentan-1-yl}-4-[3-(methylsulfanyl)propanoyl]-3,4-dihydro-2H-1,4-benzoxazine-2-carboxamide;
6-chloro-N-{3-[2-(4-chloro-3-fluorophenoxy)acetamido]bicyclo[1.1.1]pentan-1-yl}-4-[5-methyl-2-(trifluoromethyl)furan-3-sulfonyl]-3,4-dihydro-2H-1,4-benzoxazine-2-carboxamide;
6-chloro-N-{3-[2-(4-chloro-3-fluorophenoxy)acetamido]bicyclo[1.1.1]pentan-1-yl}-4-(4-methoxybenzene-1-sulfonyl)-3,4-dihydro-2H-1,4-benzoxazine-2-carboxamide;
6-chloro-N-{3-[2-(4-chloro-3-fluorophenoxy)acetamido]bicyclo[1.1.1]pentan-1-yl}-4-(1-methyl-1H-imidazole-4-sulfonyl)-3,4-dihydro-2H-1,4-benzoxazine-2-carboxamide;
6-chloro-N-{3-[2-(4-chloro-3-fluorophenoxy)acetamido]bicyclo[1.1.1]pentan-1-yl}-4-(4-fluorobenzene-1-sulfonyl)-3,4-dihydro-2H-1,4-benzoxazine-2-carboxamide;
6-chloro-N-{3-[2-(4-chloro-3-fluorophenoxy)acetamido]bicyclo[1.1.1]pentan-1-yl}-4-(5,5,5-trifluoropentanoyl)-3,4-dihydro-2H-1,4-benzoxazine-2-carboxamide;
6-chloro-N-{3-[2-(4-chloro-3-fluorophenoxy)acetamido]bicyclo[1.1.1]pentan-1-yl}-4-(methanesulfonyl)-3,4-dihydro-2H-1,4-benzoxazine-2-carboxamide;
6-chloro-N-{3-[2-(4-chloro-3-fluorophenoxy)acetamido]bicyclo[1.1.1]pentan-1-yl}-4-(pyridine-4-carbonyl)-3,4-dihydro-2H-1,4-benzoxazine-2-carboxamide;
3-[6-chloro-2-({3-[2-(4-chloro-3-fluorophenoxy)acetamido]bicyclo[1.1.1]pentan-1-yl}carbamoyl)-2,3-dihydro-4H-1,4-benzoxazin-4-yl]propanoic acid;
6-chloro-N-{3-[2-(4-chloro-3-fluorophenoxy)acetamido]bicyclo[1.1.1]pentan-1-yl}-4-[(trifluoromethoxy)acetyl]-3,4-dihydro-2H-1,4-benzoxazine-2-carboxamide;
6-chloro-N-{3-[2-(4-chloro-3-fluorophenoxy)acetamido]bicyclo[1.1.1]pentan-1-yl}-4-(methylsulfanyl)-3,4-dihydro-2H-1,4-benzoxazine-2-carboxamide;
6-chloro-N-{3-[2-(4-chloro-3-fluorophenoxy)acetamido]bicyclo[1.1.1]pentan-1-yl}-4-(1,3-dimethyl-1H-pyrazole-4-sulfonyl)-3,4-dihydro-2H-1,4-benzoxazine-2-carboxamide;
6-chloro-N-{3-[2-(4-chloro-3-fluorophenoxy)acetamido]bicyclo[1.1.1]pentan-1-yl}-4-(4-sulfamoylbenzene-1-sulfonyl)-3,4-dihydro-2H-1,4-benzoxazine-2-carboxamide;
6-chloro-N-{3-[2-(4-chloro-3-fluorophenoxy)acetamido]bicyclo[1.1.1]pentan-1-yl}-4-[(1S,2S)-2-fluorocyclopropane-1-carbonyl]-3,4-dihydro-2H-1,4-benzoxazine-2-carboxamide;
6-chloro-N-{3-[2-(4-chloro-3-fluorophenoxy)acetamido]bicyclo[1.1.1]pentan-1-yl}-4-(2,2-difluorocyclopropane-1-carbonyl)-3,4-dihydro-2H-1,4-benzoxazine-2-carboxamide;
6-chloro-N-{3-[2-(4-chloro-3-fluorophenoxy)acetamido]bicyclo[1.1.1]pentan-1-yl}-4-[1-(trifluoromethyl)cyclopropane-1-carbonyl]-3,4-dihydro-2H-1,4-benzoxazine-2-carboxamide;
6-chloro-N-{3-[2-(4-chloro-3-fluorophenoxy)acetamido]bicyclo[1.1.1]pentan-1-yl}-4-(2,2,3,3,3-pentafluoropropanoyl)-3,4-dihydro-2H-1,4-benzoxazine-2-carboxamide;
6-chloro-N-{3-[2-(4-chloro-3-fluorophenoxy)acetamido]bicyclo[1.1.1]pentan-1-yl}-4-(2,2-difluoro-1-methylcyclopropane-1-carbonyl)-3,4-dihydro-2H-1,4-benzoxazine-2-carboxamide;
6-chloro-N-{3-[2-(4-chloro-3-fluorophenoxy)acetamido]bicyclo[1.1.1]pentan-1-yl}-4-[2-(methanesulfonyl)ethyl]-3,4-dihydro-2H-1,4-benzoxazine-2-carboxamide;
6-chloro-N-{3-[2-(4-chloro-3-fluorophenoxy)acetamido]bicyclo[1.1.1]pentan-1-yl}-4-(3-methoxypropanoyl)-3,4-dihydro-2H-1,4-benzoxazine-2-carboxamide;
6-chloro-N-{3-[2-(4-chloro-3-fluorophenoxy)acetamido]bicyclo[1.1.1]pentan-1-yl}-4-(3,3,3-trifluoropropanoyl)-3,4-dihydro-2H-1,4-benzoxazine-2-carboxamide;
6-chloro-N-{3-[2-(4-chloro-3-fluorophenoxy)acetamido]bicyclo[1.1.1]pentan-1-yl}-4-(2,2,2-trifluoroethanesulfonyl)-3,4-dihydro-2H-1,4-benzoxazine-2-carboxamide;
tert-butyl [(2S)-6-chloro-2-({(3S)-4-[2-(4-chloro-3-fluorophenoxy)acetamido]-3-hydroxybicyclo[2.2.2]octan-1-yl}carbamoyl)-2,3-dihydro-4H-1,4-benzoxazin-4-yl]acetate;
tert-butyl [6-chloro-2-({(3S)-4-[2-(4-chloro-3-fluorophenoxy)acetamido]-3-hydroxybicyclo[2.2.2]octan-1-yl}carbamoyl)-2,3-dihydro-4H-1,4-benzoxazin-4-yl]acetate;
6-chloro-N-{3-[2-(4-chloro-3-fluorophenoxy)acetamido]bicyclo[1.1.1]pentan-1-yl}-4-oxo-3,4-dihydro-2H-1-benzopyran-2-carboxamide;
6-chloro-N-{3-[2-(4-chloro-3-fluorophenoxy)acetamido]bicyclo[1.1.1]pentan-1-yl}-4-(2-methoxy-2-methylpropyl)-3,4-dihydro-2H-1,4-benzoxazine-2-carboxamide;
6-chloro-N-{3-[2-(4-chloro-3-fluorophenoxy)acetamido]bicyclo[1.1.1]pentan-1-yl}-4-[(2-hydroxyethyl)amino]-3,4-dihydro-2H-1-benzopyran-2-carboxamide;
methyl {[6-chloro-2-({3-[2-(4-chloro-3-fluorophenoxy)acetamido]bicyclo[1.1.1]pentan-1-yl}carbamoyl)-3,4-dihydro-2H-1-benzopyran-4-yl]amino}acetate;
rac-(2R,4R)-6-chloro-N-{3-[2-(4-chloro-3-fluorophenoxy)acetamido]bicyclo[1.1.1]pentan-1-yl}-4-hydroxy-3,4-dihydro-2H-1-benzopyran-2-carboxamide;
(2S)-6-chloro-N-{3-[2-(4-chloro-3-fluorophenoxy)acetamido]bicyclo[1.1.1]pentan-1-yl}-4-(2-methoxy-2-methylpropyl)-3,4-dihydro-2H-1,4-benzoxazine-2-carboxamide;
{[6-chloro-2-({3-[2-(4-chloro-3-fluorophenoxy)acetamido]bicyclo[1.1.1]pentan-1-yl}carbamoyl)-3,4-dihydro-2H-1-benzopyran-4-yl]amino}acetic acid;
(2S)-6-chloro-N-{3-[2-(4-chloro-3-fluorophenoxy)acetamido]bicyclo[1.1.1]pentan-1-yl}-4-(2-methylprop-2-en-1-yl)-3,4-dihydro-2H-1,4-benzoxazine-2-carboxamide;
tert-butyl [(2R)-6-chloro-2-({(3S)-4-[2-(4-chloro-3-fluorophenoxy)acetamido]-3-hydroxybicyclo[2.2.2]octan-1-yl}carbamoyl)-2,3-dihydro-4H-1,4-benzoxazin-4-yl]acetate;
(2S,4S)-6-chloro-N-{3-[2-(4-chloro-3-fluorophenoxy)acetamido]bicyclo[1.1.1]pentan-1-yl}-4-hydroxy-3,4-dihydro-2H-1-benzopyran-2-carboxamide;
(2R,4R)-6-chloro-N-{3-[2-(4-chloro-3-fluorophenoxy)acetamido]bicyclo[1.1.1]pentan-1-yl}-4-hydroxy-3,4-dihydro-2H-1-benzopyran-2-carboxamide;
(2R,4S)-6-chloro-N-{3-[2-(4-chloro-3-fluorophenoxy)acetamido]bicyclo[1.1.1]pentan-1-yl}-4-hydroxy-3,4-dihydro-2H-1-benzopyran-2-carboxamide;
(2S,4R)-6-chloro-N-{3-[2-(4-chloro-3-fluorophenoxy)acetamido]bicyclo[1.1.1]pentan-1-yl}-4-hydroxy-3,4-dihydro-2H-1-benzopyran-2-carboxamide;
6-chloro-N-{(3S)-4-[2-(4-chloro-3-fluorophenoxy)acetamido]-3-hydroxybicyclo[2.2.2]octan-1-yl}-4-hydroxy-3,4-dihydro-2H-1-benzopyran-2-carboxamide;
N-{(2S)-4-[2-(4-chloro-3-fluorophenoxy)acetamido]-2-hydroxybicyclo[2.2.2]octan-1-yl}-6-fluoro-3,4-dihydro-2H-1-benzopyran-2-carboxamide;
(2S)—N-{(2S)-4-[2-(4-chloro-3-fluorophenoxy)acetamido]-2-hydroxybicyclo[2.2.2]octan-1-yl}-6-fluoro-3,4-dihydro-2H-1-benzopyran-2-carboxamide;
(2R)—N-{(2S)-4-[2-(4-chloro-3-fluorophenoxy)acetamido]-2-hydroxybicyclo[2.2.2]octan-1-yl}-6-fluoro-3,4-dihydro-2H-1-benzopyran-2-carboxamide;
6-chloro-N-{(2S)-4-[2-(4-chloro-3-fluorophenoxy)acetamido]-2-hydroxybicyclo[2.2.2]octan-1-yl}-3,4-dihydro-2H-1-benzopyran-2-carboxamide;
6-chloro-N-{3-[2-(4-chloro-3-fluorophenoxy)acetamido]bicyclo[1.1.1]pentan-1-yl}-3,4-dihydro-2H-1-benzopyran-2-carboxamide;
(2S,4S)-7-chloro-N-{3-[2-(4-chloro-3-fluorophenoxy)acetamido]bicyclo[1.1.1]pentan-1-yl}-6-fluoro-4-hydroxy-3,4-dihydro-2H-1-benzopyran-2-carboxamide;
(2R,4R)-7-chloro-N-{3-[2-(4-chloro-3-fluorophenoxy)acetamido]bicyclo[1.1.1]pentan-1-yl}-6-fluoro-4-hydroxy-3,4-dihydro-2H-1-benzopyran-2-carboxamide;
(2R,4R)—N-{3-[2-(3,4-dichlorophenoxy)acetamido]bicyclo[1.1.1]pentan-1-yl}-6,7-difluoro-4-hydroxy-3,4-dihydro-2H-1-benzopyran-2-carboxamide;
(2S,4S)—N-{3-[2-(3,4-dichlorophenoxy)acetamido]bicyclo[1.1.1]pentan-1-yl}-6,7-difluoro-4-hydroxy-3,4-dihydro-2H-1-benzopyran-2-carboxamide;
6-chloro-N-{3-[2-(4-chloro-3-fluorophenoxy)acetamido]bicyclo[1.1.1]pentan-1-yl}-4-{[(1s,3s)-3-hydroxycyclobutyl]amino}-3,4-dihydro-2H-1-benzopyran-2-carboxamide;
1-{[6-chloro-2-({3-[2-(4-chloro-3-fluorophenoxy)acetamido]bicyclo[1.1.1]pentan-1-yl}carbamoyl)-3,4-dihydro-2H-1-benzopyran-4-yl]amino}cyclopropane-1-carboxylic acid;
methyl [(2S)-6-chloro-2-({3-[2-(4-chloro-3-fluorophenoxy)acetamido]bicyclo[1.1.1]pentan-1-yl}carbamoyl)-2,3-dihydro-4H-1,4-benzoxazin-4-yl]acetate;
6-chloro-N-{3-[2-(4-chloro-3-fluorophenoxy)acetamido]bicyclo[1.1.1]pentan-1-yl}-4-[(3,3-difluorocyclobutyl)amino]-3,4-dihydro-2H-1-benzopyran-2-carboxamide;
N-{3-[(6-chloro-4-hydroxy-3,4-dihydro-2H-1-benzopyran-2-carbonyl)amino]bicyclo[1.1.1]pentan-1-yl}-5-(difluoromethyl)pyrazine-2-carboxamide;
N-{(3S)-4-[(6-chloro-4-oxo-3,4-dihydro-2H-1-benzopyran-2-carbonyl)amino]-3-hydroxybicyclo[2.2.2]octan-1-yl}-3-(difluoromethyl)-1,2-oxazole-5-carboxamide;
N-[(3S)-4-{[(2R,4R)-6-chloro-4-hydroxy-3,4-dihydro-2H-1-benzopyran-2-carbonyl]amino}-3-hydroxybicyclo[2.2.2]octan-1-yl]-3-(difluoromethyl)-1,2-oxazole-5-carboxamide;
N-[(3S)-4-{[(2S,4S)-6-chloro-4-hydroxy-3,4-dihydro-2H-1-benzopyran-2-carbonyl]amino}-3-hydroxybicyclo[2.2.2]octan-1-yl]-3-(difluoromethyl)-1,2-oxazole-5-carboxamide;
N-{3-[(6-chloro-4-oxo-3,4-dihydro-2H-1-benzopyran-2-carbonyl)amino]bicyclo[1.1.1]pentan-1-yl}-5-(difluoromethyl)pyrazine-2-carboxamide;
N-(3-{[rac-(2R,4R)-6-chloro-4-hydroxy-3,4-dihydro-2H-1-benzopyran-2-carbonyl]amino}bicyclo[1.1.1]pentan-1-yl)-5-(difluoromethyl)pyrazine-2-carboxamide;
6-chloro-N-{3-[2-(4-chloro-3-fluorophenoxy)acetamido]bicyclo[1.1.1]pentan-1-yl}-4-[(methanesulfonyl)amino]-3,4-dihydro-2H-1-benzopyran-2-carboxamide;
4-acetamido-6-chloro-N-{3-[2-(4-chloro-3-fluorophenoxy)acetamido]bicyclo[1.1.1]pentan-1-yl}-3,4-dihydro-2H-1-benzopyran-2-carboxamide;
N-{3-[2-(4-chloro-3-fluorophenoxy)acetamido]bicyclo[1.1.1]pentan-1-yl}-6,7-dimethoxy-4-oxo-4H-1-benzopyran-2-carboxamide;
N-{3-[2-(4-chloro-3-fluorophenoxy)acetamido]bicyclo[1.1.1]pentan-1-yl}-7-fluoro-4-hydroxy-3,4-dihydro-2H-1-benzopyran-2-carboxamide;
(2S)-6-chloro-N-{3-[2-(4-chloro-3-fluorophenoxy)acetamido]bicyclo[1.1.1]pentan-1-yl}-4-oxo-3,4-dihydro-2H-1-benzopyran-2-carboxamide;
(2R)-6-chloro-4-oxo-N-[3-(2-{[6-(trifluoromethyl)pyridin-3-yl]oxy}acetamido)bicyclo[1.1.1]pentan-1-yl]-3,4-dihydro-2H-1-benzopyran-2-carboxamide;
(2R,4R)-6-chloro-4-hydroxy-N-[3-(2-{[6-(trifluoromethyl)pyridin-3-yl]oxy}acetamido)bicyclo[1.1.1]pentan-1-yl]-3,4-dihydro-2H-1-benzopyran-2-carboxamide;
(2R,4R)-6-chloro-4-hydroxy-N-(3-{2-[(2-methoxypyrimidin-5-yl)oxy]acetamido}bicyclo[1.1.1]pentan-1-yl)-3,4-dihydro-2H-1-benzopyran-2-carboxamide;
(2R,4R)-6-chloro-4-hydroxy-N-[3-(2-{[2-(trifluoromethyl)pyrimidin-5-yl]oxy}acetamido)bicyclo[1.1.1]pentan-1-yl]-3,4-dihydro-2H-1-benzopyran-2-carboxamide;
(2R,4R)-6-chloro-N-{4-[2-(4-chloro-3-fluorophenoxy)acetamido]bicyclo[2.2.2]octan-1-yl}-4-hydroxy-3,4-dihydro-2H-1-benzopyran-2-carboxamide;
N-{3-[2-(4-chloro-3-fluorophenoxy)acetamido]bicyclo[1.1.1]pentan-1-yl}-4-oxo-7-(trifluoromethyl)-4H-1-benzopyran-2-carboxamide;
6-chloro-N-{3-[2-(4-chloro-3-fluorophenoxy)acetamido]bicyclo[1.1.1]pentan-1-yl}-7-fluoro-4-oxo-4H-1-benzopyran-2-carboxamide; 7-chloro-N-{3-[2-(4-chloro-3-fluorophenoxy)acetamido]bicyclo[1.1.1]pentan-1-yl}-6-fluoro-4-oxo-4H-1-benzopyran-2-carboxamide;
(2R,4R)-6-chloro-N-(3-{2-[(5-chloropyridin-2-yl)oxy]acetamido}bicyclo[1.1.1]pentan-1-yl)-4-hydroxy-3,4-dihydro-2H-1-benzopyran-2-carboxamide;
rac-(2R,4R)-6-chloro-N-{3-[2-(4-chloro-3-fluorophenoxy)acetamido]bicyclo[1.1.1]pentan-1-yl}-7-fluoro-4-hydroxy-3,4-dihydro-2H-1-benzopyran-2-carboxamide;
7-chloro-N-{3-[2-(4-chloro-3-fluorophenoxy)acetamido]bicyclo[1.1.1]pentan-1-yl}-4-oxo-4H-1-benzopyran-2-carboxamide;
6-chloro-N-{3-[2-(4-chloro-3-fluorophenoxy)acetamido]bicyclo[1.1.1]pentan-1-yl}-7-methyl-4-oxo-4H-1-benzopyran-2-carboxamide;
(2S,4S)-6-chloro-N-{3-[2-(4-chloro-3-fluorophenoxy)acetamido]bicyclo[1.1.1]pentan-1-yl}-4-hydroxy-4-methyl-3,4-dihydro-2H-1-benzopyran-2-carboxamide;
(2R,4R)-6-chloro-N-{(2S)-4-[2-(4-chloro-3-fluorophenoxy)acetamido]-2-hydroxybicyclo[2.2.2]octan-1-yl}-4-hydroxy-3,4-dihydro-2H-1-benzopyran-2-carboxamide;
(2S,4S)-6-chloro-N-{(2S)-4-[2-(4-chloro-3-fluorophenoxy)acetamido]-2-hydroxybicyclo[2.2.2]octan-1-yl}-4-hydroxy-3,4-dihydro-2H-1-benzopyran-2-carboxamide;
6-chloro-N-{3-[2-(4-chloro-3-fluorophenoxy)acetamido]bicyclo[1.1.1]pentan-1-yl}-2-methyl-3,4-dihydro-2H-1-benzopyran-2-carboxamide;
(2R,4R)—N-{3-[2-(4-chloro-3-fluorophenoxy)acetamido]bicyclo[1.1.1]pentan-1-yl}-6-fluoro-4-hydroxy-7-methyl-3,4-dihydro-2H-1-benzopyran-2-carboxamide;
(2S,4S)—N-{3-[2-(4-chloro-3-fluorophenoxy)acetamido]bicyclo[1.1.1]pentan-1-yl}-6-fluoro-4-hydroxy-7-methyl-3,4-dihydro-2H-1-benzopyran-2-carboxamide;
N-{3-[2-(4-chloro-3-fluorophenoxy)acetamido]bicyclo[1.1.1]pentan-1-yl}-4-hydroxy-7-(trifluoromethyl)-3,4-dihydro-2H-1-benzopyran-2-carboxamide;
(2R,4S)-6-chloro-N-{3-[2-(4-chloro-3-fluorophenoxy)acetamido]bicyclo[1.1.1]pentan-1-yl}-4-[(methanesulfonyl)amino]-3,4-dihydro-2H-1-benzopyran-2-carboxamide;
(2S,4R)-6-chloro-N-{3-[2-(4-chloro-3-fluorophenoxy)acetamido]bicyclo[1.1.1]pentan-1-yl}-4-[(methanesulfonyl)amino]-3,4-dihydro-2H-1-benzopyran-2-carboxamide;
7-chloro-N-{3-[2-(4-chloro-3-fluorophenoxy)acetamido]bicyclo[1.1.1]pentan-1-yl}-6-fluoro-4-hydroxy-3,4-dihydro-2H-1-benzopyran-2-carboxamide;
7-chloro-N-{3-[2-(4-chloro-3-fluorophenoxy)acetamido]bicyclo[1.1.1]pentan-1-yl}-4-hydroxy-3,4-dihydro-2H-1-benzopyran-2-carboxamide;
N-{3-[2-(4-chloro-3-fluorophenoxy)acetamido]bicyclo[1.1.1]pentan-1-yl}-6,7-difluoro-4-oxo-3,4-dihydro-2H-1-benzopyran-2-carboxamide;
rac-(2R,4R)—N-{3-[2-(4-chloro-3-fluorophenoxy)acetamido]bicyclo[1.1.1]pentan-1-yl}-6,7-difluoro-4-hydroxy-3,4-dihydro-2H-1-benzopyran-2-carboxamide;
rac-(2R,4S)-6-chloro-N-{3-[2-(4-chloro-3-fluorophenoxy)acetamido]bicyclo[1.1.1]pentan-1-yl}-4-hydroxy-2-methyl-3,4-dihydro-2H-1-benzopyran-2-carboxamide;
rac-(2R,4R)-6-chloro-N-{3-[2-(4-chloro-3-fluorophenoxy)acetamido]bicyclo[1.1.1]pentan-1-yl}-4-hydroxy-2-methyl-3,4-dihydro-2H-1-benzopyran-2-carboxamide;
(2R,4R)—N-{3-[2-(4-chloro-3-fluorophenoxy)acetamido]bicyclo[1.1.1]pentan-1-yl}-6,7-difluoro-4-hydroxy-3,4-dihydro-2H-1-benzopyran-2-carboxamide;
(2S,4S)—N-{3-[2-(4-chloro-3-fluorophenoxy)acetamido]bicyclo[1.1.1]pentan-1-yl}-6,7-difluoro-4-hydroxy-3,4-dihydro-2H-1-benzopyran-2-carboxamide;
N-{3-[2-(4-chloro-3-fluorophenoxy)acetamido]bicyclo[1.1.1]pentan-1-yl}-4-hydroxy-6-methyl-3,4-dihydro-2H-1-benzopyran-2-carboxamide;
(2R)-6-chloro-N-{4-[2-(4-chloro-3-fluorophenoxy)acetamido]bicyclo[2.1.1]hexan-1-yl}-4-oxo-3,4-dihydro-2H-1-benzopyran-2-carboxamide;
(2R,4R)-6-chloro-N-{4-[2-(4-chloro-3-fluorophenoxy)acetamido]bicyclo[2.1.1]hexan-1-yl}-4-hydroxy-3,4-dihydro-2H-1-benzopyran-2-carboxamide;
(2S)-5-chloro-N-{3-[2-(4-chloro-3-fluorophenoxy)acetamido]bicyclo[1.1.1]pentan-1-yl}-2-methyl-2,3-dihydro-1-benzofuran-2-carboxamide;
(2R)-5-chloro-N-{3-[2-(4-chloro-3-fluorophenoxy)acetamido]bicyclo[1.1.1]pentan-1-yl}-2-methyl-2,3-dihydro-1-benzofuran-2-carboxamide;
(2S)-5-chloro-N-{3-[2-(4-chloro-3-fluorophenoxy)acetamido]bicyclo[1.1.1]pentan-1-yl}-2-methyl-3-oxo-2,3-dihydro-1-benzofuran-2-carboxamide;
(2R,4S)-6-chloro-N-{4-[2-(4-chloro-3-fluorophenoxy)acetamido]bicyclo[2.1.1]hexan-1-yl}-4-hydroxy-3,4-dihydro-2H-1-benzopyran-2-carboxamide;
5-chloro-N-{3-[2-(4-chloro-3-fluorophenoxy)acetamido]bicyclo[1.1.1]pentan-1-yl}-2,3-dihydro-1H-indole-2-carboxamide;
(2S)-5-chloro-N-{3-[2-(4-chloro-3-fluorophenoxy)acetamido]bicyclo[1.1.1]pentan-1-yl}-3-hydroxy-2-methyl-2,3-dihydro-1-benzofuran-2-carboxamide;
(2R)-5-chloro-N-{3-[2-(4-chloro-3-fluorophenoxy)acetamido]bicyclo[1.1.1]pentan-1-yl}-3-hydroxy-2-methyl-2,3-dihydro-1-benzofuran-2-carboxamide;
(2R,4R)-6-chloro-N-{3-[2-(4-chloro-3-fluorophenoxy)acetamido]bicyclo[1.1.1]pentan-1-yl}-7-fluoro-4-hydroxy-3,4-dihydro-2H-1-benzopyran-2-carboxamide;
(2S,4S)-6-chloro-N-{3-[2-(4-chloro-3-fluorophenoxy)acetamido]bicyclo[1.1.1]pentan-1-yl}-7-fluoro-4-hydroxy-3,4-dihydro-2H-1-benzopyran-2-carboxamide;
5-chloro-N-{3-[2-(4-chloro-3-fluorophenoxy)acetamido]bicyclo[1.1.1]pentan-1-yl}-1-methyl-2,3-dihydro-1H-indole-2-carboxamide;
(2R,4R)—N-{3-[2-(4-chloro-3-fluorophenoxy)acetamido]bicyclo[1.1.1]pentan-1-yl}-4-hydroxy-3,4-dihydro-2H-1-benzopyran-2-carboxamide;
(2R)—N-{3-[2-(4-chloro-3-fluorophenoxy)acetamido]bicyclo[1.1.1]pentan-1-yl}-4-oxo-3,4-dihydro-2H-1-benzopyran-2-carboxamide;
(2S)—N-{3-[2-(4-chloro-3-fluorophenoxy)acetamido]bicyclo[1.1.1]pentan-1-yl}-4-oxo-3,4-dihydro-2H-1-benzopyran-2-carboxamide;
(2S,4S)—N-{3-[2-(4-chloro-3-fluorophenoxy)acetamido]bicyclo[1.1.1]pentan-1-yl}-4-hydroxy-3,4-dihydro-2H-1-benzopyran-2-carboxamide;
(2R)—N-{3-[2-(3,4-difluorophenoxy)acetamido]bicyclo[1.1.1]pentan-1-yl}-6,7-difluoro-4-oxo-3,4-dihydro-2H-1-benzopyran-2-carboxamide;
(2R,4R)—N-{3-[2-(3,4-difluorophenoxy)acetamido]bicyclo[1.1.1]pentan-1-yl}-6,7-difluoro-4-hydroxy-3,4-dihydro-2H-1-benzopyran-2-carboxamide;
(2S,4S)—N-{3-[2-(3,4-difluorophenoxy)acetamido]bicyclo[1.1.1]pentan-1-yl}-6,7-difluoro-4-hydroxy-3,4-dihydro-2H-1-benzopyran-2-carboxamide;
N-[(2S)-4-{[(2R,4R)-6-chloro-4-hydroxy-3,4-dihydro-2H-1-benzopyran-2-carbonyl]amino}-2-hydroxybicyclo[2.2.2]octan-1-yl]-2-ethyl-1,3-oxazole-5-carboxamide;
(2R)-6-chloro-N-{3-[2-(3,4-difluorophenoxy)acetamido]bicyclo[1.1.1]pentan-1-yl}-4-oxo-3,4-dihydro-2H-1-benzopyran-2-carboxamide;
(2S)-6-chloro-N-{3-[2-(3,4-difluorophenoxy)acetamido]bicyclo[1.1.1]pentan-1-yl}-4-oxo-3,4-dihydro-2H-1-benzopyran-2-carboxamide;
(2R,4R)-6-chloro-N-{3-[2-(3,4-difluorophenoxy)acetamido]bicyclo[1.1.1]pentan-1-yl}-4-hydroxy-3,4-dihydro-2H-1-benzopyran-2-carboxamide;
(2S,4S)-6-chloro-N-{3-[2-(3,4-difluorophenoxy)acetamido]bicyclo[1.1.1]pentan-1-yl}-4-hydroxy-3,4-dihydro-2H-1-benzopyran-2-carboxamide;
(2S)-6-chloro-N-{3-[2-(4-chloro-3-fluorophenoxy)acetamido]bicyclo[1.1.1]pentan-1-yl}-2H-1-benzopyran-2-carboxamide;
(2R,4R)-6-chloro-N-{3-[2-(4-chloro-3-fluorophenoxy)acetamido]bicyclo[1.1.1]pentan-1-yl}-4-hydroxy-4-methyl-3,4-dihydro-2H-1-benzopyran-2-carboxamide;
(2R,4S)-6-chloro-N-{3-[2-(4-chloro-3-fluorophenoxy)acetamido]bicyclo[1.1.1]pentan-1-yl}-4-hydroxy-4-methyl-3,4-dihydro-2H-1-benzopyran-2-carboxamide;
6-chloro-N-{3-[2-(4-chloro-3-fluorophenoxy)acetamido]bicyclo[1.1.1]pentan-1-yl}-8-methyl-4-oxo-4H-1-benzopyran-2-carboxamide;
N-{3-[2-(4-chloro-3-fluorophenoxy)acetamido]bicyclo[1.1.1]pentan-1-yl}-7-methoxy-4-oxo-4H-1-benzopyran-2-carboxamide;
N-{3-[2-(4-chloro-3-fluorophenoxy)acetamido]bicyclo[1.1.1]pentan-1-yl}-7-methyl-4-oxo-4H-1-benzopyran-2-carboxamide;
N-{3-[2-(4-chloro-3-fluorophenoxy)acetamido]bicyclo[1.1.1]pentan-1-yl}-6,7-dimethyl-4-oxo-4H-1-benzopyran-2-carboxamide;
N-{3-[2-(4-chloro-3-fluorophenoxy)acetamido]bicyclo[1.1.1]pentan-1-yl}-7-(difluoromethoxy)-4-oxo-4H-1-benzopyran-2-carboxamide;
N-{3-[2-(4-chloro-3-fluorophenoxy)acetamido]bicyclo[1.1.1]pentan-1-yl}-6-methoxy-4-oxo-4H-1-benzopyran-2-carboxamide;
(2S,4R)-6-chloro-N-{3-[2-(4-chloro-3-fluorophenoxy)acetamido]bicyclo[1.1.1]pentan-1-yl}-4-hydroxy-4-methyl-3,4-dihydro-2H-1-benzopyran-2-carboxamide;
6,8-dichloro-N-{3-[2-(4-chloro-3-fluorophenoxy)acetamido]bicyclo[1.1.1]pentan-1-yl}-4-oxo-4H-1-benzopyran-2-carboxamide;
N-{3-[2-(4-chloro-3-fluorophenoxy)acetamido]bicyclo[1.1.1]pentan-1-yl}-4-oxo-6-(propan-2-yl)-4H-1-benzopyran-2-carboxamide;
N-{3-[2-(4-chloro-3-fluorophenoxy)acetamido]bicyclo[1.1.1]pentan-1-yl}-6-oxo-2H,6H-[1,3]dioxolo[4,5-h][1]benzopyran-8-carboxamide;
N-{3-[2-(4-chloro-3-fluorophenoxy)acetamido]bicyclo[1.1.1]pentan-1-yl}-6,8-difluoro-4-oxo-4H-1-benzopyran-2-carboxamide;
N-(4-{[rac-(2R,4R)-6-chloro-4-hydroxy-3,4-dihydro-2H-1-benzopyran-2-carbonyl]amino}bicyclo[2.1.1]hexan-1-yl)-5-(trifluoromethoxy)pyridine-2-carboxamide;
8-chloro-N-{3-[2-(4-chloro-3-fluorophenoxy)acetamido]bicyclo[1.1.1]pentan-1-yl}-4-oxo-4H-1-benzopyran-2-carboxamide;
N-{3-[2-(4-chloro-3-fluorophenoxy)acetamido]bicyclo[1.1.1]pentan-1-yl}-7-hydroxy-4-oxo-4H-1-benzopyran-2-carboxamide;
rac-(2R,4R)-6-chloro-N-{4-[2-(4-chloro-3-fluorophenoxy)acetamido]bicyclo[2.1.1]hexan-1-yl}-4-hydroxy-3,4-dihydro-2H-1-benzopyran-2-carboxamide;
6-chloro-N-{3-[2-(4-chloro-3-fluorophenoxy)acetamido]bicyclo[1.1.1]pentan-1-yl}-4-ethyl-4-hydroxy-3,4-dihydro-2H-1-benzopyran-2-carboxamide;
N-{3-[2-(4-chloro-3-fluorophenoxy)acetamido]bicyclo[1.1.1]pentan-1-yl}-6-fluoro-4-(hydroxyimino)-3,4-dihydro-2H-1-benzopyran-2-carboxamide;
(2S,4S)-6-chloro-4-hydroxy-N-[3-(2-{[6-(trifluoromethyl)pyridin-3-yl]oxy}acetamido)bicyclo[1.1.1]pentan-1-yl]-3,4-dihydro-2H-1-benzopyran-2-carboxamide;
N-{3-[2-(4-chloro-3-fluorophenoxy)acetamido]bicyclo[1.1.1]pentan-1-yl}-6-fluoro-4-hydroxy-3,4-dihydro-2H-1-benzopyran-2-carboxamide;
rac-(2R,4S)-6-chloro-N-{3-[2-(4-chloro-3-fluorophenoxy)acetamido]bicyclo[1.1.1]pentan-1-yl}-4-[(2-hydroxyethyl)amino]-3,4-dihydro-2H-1-benzopyran-2-carboxamide;
N-(4-{[rac-(2R,4R)-6-chloro-4-hydroxy-3,4-dihydro-2H-1-benzopyran-2-carbonyl]amino}bicyclo[2.2.2]octan-1-yl)-5-(difluoromethyl)pyrazine-2-carboxamide;
6-chloro-N-{(3S)-4-[2-(4-chloro-3-fluorophenoxy)acetamido]-3-hydroxybicyclo[2.2.2]octan-1-yl}-4-(methylamino)-3,4-dihydro-2H-1-benzopyran-2-carboxamide;
N-{4-[(6-chloro-4-oxo-3,4-dihydro-2H-1-benzopyran-2-carbonyl)amino]bicyclo[2.2.2]octan-1-yl}-5-(difluoromethyl)pyrazine-2-carboxamide;
4-amino-6-chloro-N-{3-[2-(4-chloro-3-fluorophenoxy)acetamido]bicyclo[1.1.1]pentan-1-yl}-3,4-dihydro-2H-1-benzopyran-2-carboxamide;
(2S,4S)-6-chloro-N-{4-[2-(4-chloro-3-fluorophenoxy)acetamido]bicyclo[2.2.2]octan-1-yl}-4-hydroxy-3,4-dihydro-2H-1-benzopyran-2-carboxamide;
(2R)-6-chloro-N-{3-[2-(4-chloro-3-fluorophenoxy)acetamido]bicyclo[1.1.1]pentan-1-yl}-2H-1-benzopyran-2-carboxamide;
(2R,4R)-6-chloro-N-{3-[2-(4-chloro-3-fluorophenoxy)acetamido]bicyclo[1.1.1]pentan-1-yl}-4-[(2-hydroxyethyl)amino]-3,4-dihydro-2H-1-benzopyran-2-carboxamide;
(2S,4S)-6-chloro-N-{3-[2-(4-chloro-3-fluorophenoxy)acetamido]bicyclo[1.1.1]pentan-1-yl}-4-[(2-hydroxyethyl)amino]-3,4-dihydro-2H-1-benzopyran-2-carboxamide;
6-chloro-N-{4-[2-(4-chloro-3-fluorophenoxy)acetamido]bicyclo[2.1.1]hexan-1-yl}-4-oxo-3,4-dihydro-2H-1-benzopyran-2-carboxamide;
N-{4-[(6-chloro-4-oxo-3,4-dihydro-2H-1-benzopyran-2-carbonyl)amino]bicyclo[2.1.1]hexan-1-yl}-5-(trifluoromethoxy)pyridine-2-carboxamide;
benzyl {4-[(6-chloro-4-oxo-3,4-dihydro-2H-1-benzopyran-2-carbonyl)amino]bicyclo[2.1.1]hexan-1-yl}carbamate;
N-{3-[2-(4-chloro-3-fluorophenoxy)acetamido]bicyclo[1.1.1]pentan-1-yl}-7-fluoro-4-oxo-4H-1-benzopyran-2-carboxamide;
6-chloro-N-{3-[2-(4-chloro-3-fluorophenoxy)acetamido]bicyclo[1.1.1]pentan-1-yl}-4-(methylamino)-3,4-dihydro-2H-1-benzopyran-2-carboxamide;
6-chloro-N-{3-[2-(4-chloro-3-fluorophenoxy)acetamido]bicyclo[1.1.1]pentan-1-yl}-4-hydroxy-4H-1-benzopyran-2-carboxamide;
(2S,4R)-6-chloro-N-{3-[2-(4-chloro-3-fluorophenoxy)acetamido]bicyclo[1.1.1]pentan-1-yl}-4-{[(1s,3S)-3-hydroxycyclobutyl]amino}-3,4-dihydro-2H-1-benzopyran-2-carboxamide;
7-chloro-N-{3-[2-(4-chloro-3-fluorophenoxy)acetamido]bicyclo[1.1.1]pentan-1-yl}-2H-1-benzopyran-3-carboxamide;
6-chloro-N-{3-[2-(4-chloro-3-fluorophenoxy)acetamido]bicyclo[1.1.1]pentan-1-yl}-4-oxo-4H-1-benzopyran-2-carboxamide;
(2S,4S)-6-chloro-N-{3-[2-(4-chloro-3-fluorophenoxy)acetamido]bicyclo[1.1.1]pentan-1-yl}-4-[(2,2,2-trifluoroethyl)amino]-3,4-dihydro-2H-1-benzopyran-2-carboxamide;
(2R)-5-chloro-N-{3-[2-(4-chloro-3-fluorophenoxy)acetamido]bicyclo[1.1.1]pentan-1-yl}-2-methyl-3-oxo-2,3-dihydro-1-benzofuran-2-carboxamide;
(2S)—N-{3-[2-(3,4-difluorophenoxy)acetamido]bicyclo[1.1.1]pentan-1-yl}-6,7-difluoro-4-oxo-3,4-dihydro-2H-1-benzopyran-2-carboxamide;
N-{3-[2-(4-chloro-3-fluorophenoxy)acetamido]bicyclo[1.1.1]pentan-1-yl}-6-methoxy-4-oxo-4H-pyrano[3,2-b]pyridine-2-carboxamide;
(2S,4R)-6-chloro-N-{3-[2-(4-chloro-3-fluorophenoxy)acetamido]bicyclo[1.1.1]pentan-1-yl}-4-[(2,2,2-trifluoroethyl)amino]-3,4-dihydro-2H-1-benzopyran-2-carboxamide;
6-chloro-N-{3-[2-(4-chloro-3-fluorophenoxy)acetamido]bicyclo[1.1.1]pentan-1-yl}-N-methyl-4-oxo-4H-1-benzopyran-2-carboxamide;
(2R)-6-chloro-N-{3-[2-(4-chloro-3-fluorophenoxy)acetamido]bicyclo[1.1.1]pentan-1-yl}-4-[(1-hydroxycyclopropyl)methyl]-3,4-dihydro-2H-1,4-benzoxazine-2-carboxamide;
6-chloro-N-{4-[2-(4-chloro-3-fluorophenoxy)acetamido]bicyclo[2.2.2]octan-1-yl}-4-oxo-3,4-dihydro-2H-1-benzopyran-2-carboxamide;
(2R,4S)-6-chloro-N-{3-[2-(4-chloro-3-fluorophenoxy)acetamido]bicyclo[1.1.1]pentan-1-yl}-4-[(2,2,2-trifluoroethyl)amino]-3,4-dihydro-2H-1-benzopyran-2-carboxamide;
(2R,4R)-6-chloro-N-{3-[2-(4-chloro-3-fluorophenoxy)acetamido]bicyclo[1.1.1]pentan-1-yl}-4-{[(1s,3S)-3-hydroxycyclobutyl]amino}-3,4-dihydro-2H-1-benzopyran-2-carboxamide;
(2R)-6-chloro-4-oxo-N-[3-(2-{[2-(trifluoromethyl)pyrimidin-5-yl]oxy}acetamido)bicyclo[1.1.1]pentan-1-yl]-3,4-dihydro-2H-1-benzopyran-2-carboxamide;
N-{3-[2-(4-chloro-3-fluorophenoxy)acetamido]bicyclo[1.1.1]pentan-1-yl}-6-fluoro-4-methoxy-3,4-dihydro-2H-1-benzopyran-2-carboxamide;
(2R)-6-chloro-N-(3-{2-[(5-chloropyridin-2-yl)oxy]acetamido}bicyclo[1.1.1]pentan-1-yl)-4-oxo-3,4-dihydro-2H-1-benzopyran-2-carboxamide;
N-{3-[2-(4-chloro-3-fluorophenoxy)acetamido]bicyclo[1.1.1]pentan-1-yl}-6-fluoro-4-oxo-3,4-dihydro-2H-1-benzopyran-2-carboxamide;
(2R)-6-chloro-N-(3-{2-[(2-methoxypyrimidin-5-yl)oxy]acetamido}bicyclo[1.1.1]pentan-1-yl)-4-oxo-3,4-dihydro-2H-1-benzopyran-2-carboxamide;
rac-(2R,4R)—N-{3-[2-(4-chloro-3-fluorophenoxy)acetamido]bicyclo[1.1.1]pentan-1-yl}-4-hydroxy-6-methoxy-3,4-dihydro-2H-pyrano[3,2-b]pyridine-2-carboxamide;
(2S)-6-chloro-4-oxo-N-[3-(2-{[6-(trifluoromethyl)pyridin-3-yl]oxy}acetamido)bicyclo[1.1.1]pentan-1-yl]-3,4-dihydro-2H-1-benzopyran-2-carboxamide;
6-chloro-N-{3-[2-(4-chloro-3-fluorophenoxy)acetamido]bicyclo[1.1.1]pentan-1-yl}-4-[(2,2,2-trifluoroethyl)amino]-3,4-dihydro-2H-1-benzopyran-2-carboxamide;
6-chloro-N-{4-[2-(4-chloro-3-fluorophenoxy)acetamido]bicyclo[2.2.2]octan-1-yl}-4-hydroxy-3,4-dihydro-2H-1-benzopyran-2-carboxamide;
6-chloro-N-{3-[2-(4-chloro-3-fluorophenoxy)acetamido]bicyclo[1.1.1]pentan-1-yl}-2-methyl-4-oxo-3,4-dihydro-2H-1-benzopyran-2-carboxamide;
6-chloro-N-{3-[2-(4-chloro-3-fluorophenoxy)acetamido]bicyclo[1.1.1]pentan-1-yl}-4-{[1-(hydroxymethyl)cyclopropyl]amino}-3,4-dihydro-2H-1-benzopyran-2-carboxamide;
rac-(2R,4R)-6-chloro-N-{3-[2-(4-chloro-3-fluorophenoxy)acetamido]bicyclo[1.1.1]pentan-1-yl}-4-methoxy-3,4-dihydro-2H-1-benzopyran-2-carboxamide;
N-{3-[2-(4-chloro-3-fluorophenoxy)acetamido]bicyclo[1.1.1]pentan-1-yl}-6-methyl-4-oxo-4H-1-benzopyran-2-carboxamide;
(2S)—N-{3-[2-(3,4-dichlorophenoxy)acetamido]bicyclo[1.1.1]pentan-1-yl}-6,7-difluoro-4-oxo-3,4-dihydro-2H-1-benzopyran-2-carboxamide;
rac-(2R,4S)-6-chloro-N-{3-[2-(4-chloro-3-fluorophenoxy)acetamido]bicyclo[1.1.1]pentan-1-yl}-4-{[1-(hydroxymethyl)cyclopropyl]amino}-3,4-dihydro-2H-1-benzopyran-2-carboxamide;
N-[(3S)-4-{[(2R,4R)-6-chloro-4-hydroxy-3,4-dihydro-2H-1-benzopyran-2-carbonyl]amino}-3-hydroxybicyclo[2.2.2]octan-1-yl]-4-methyl-1,3-thiazole-2-carboxamide;
7-chloro-N-{3-[2-(4-chloro-3-fluorophenoxy)acetamido]bicyclo[1.1.1]pentan-1-yl}-3,4-dihydro-2H-1-benzopyran-3-carboxamide;
N-{3-[2-(4-chloro-3-fluorophenoxy)acetamido]bicyclo[1.1.1]pentan-1-yl}-6-hydroxy-4-oxo-4H-1-benzopyran-2-carboxamide;
N-{3-[2-(4-chloro-3-fluorophenoxy)acetamido]bicyclo[1.1.1]pentan-1-yl}-6-fluoro-4-hydroxy-7-methyl-3,4-dihydro-2H-1-benzopyran-2-carboxamide;
(2S)-6-chloro-N-{3-[2-(4-chloro-3-fluorophenoxy)acetamido]bicyclo[1.1.1]pentan-1-yl}-4-(2-hydroxy-2-methylpropyl)-3,4-dihydro-2H-1,4-benzoxazine-2-carboxamide;
6-chloro-N-{3-[2-(4-chloro-3-fluorophenoxy)acetamido]bicyclo[1.1.1]pentan-1-yl}-4-[(oxetan-3-yl)amino]-3,4-dihydro-2H-1-benzopyran-2-carboxamide;
6-chloro-N-{3-[2-(4-chloro-3-fluorophenoxy)acetamido]bicyclo[1.1.1]pentan-1-yl}-2-fluoro-4-hydroxy-3,4-dihydro-2H-1-benzopyran-2-carboxamide;
(2R,4R)-6-chloro-N-(3-{2-[(4-fluoro-1H-indazol-6-yl)oxy]acetamido}bicyclo[1.1.1]pentan-1-yl)-4-hydroxy-3,4-dihydro-2H-1-benzopyran-2-carboxamide;
N-[(2S)-4-{[(2R)-6-chloro-4-oxo-3,4-dihydro-2H-1-benzopyran-2-carbonyl]amino}-2-hydroxybicyclo[2.2.2]octan-1-yl]-5-(difluoromethyl)pyrazine-2-carboxamide;
N-[(2S)-4-{[(2R,4R)-6-chloro-4-hydroxy-3,4-dihydro-2H-1-benzopyran-2-carbonyl]amino}-2-hydroxybicyclo[2.2.2]octan-1-yl]-5-(difluoromethyl)pyrazine-2-carboxamide;
N-[(2S)-4-{[(2R)-6-chloro-4-oxo-3,4-dihydro-2H-1-benzopyran-2-carbonyl]amino}-2-hydroxybicyclo[2.2.2]octan-1-yl]-4-methyl-1,3-thiazole-2-carboxamide;
(2R,4R)-6-chloro-N-[(2S)-4-{2-[(4-fluoro-1H-indazol-6-yl)oxy]acetamido}-2-hydroxybicyclo[2.2.2]octan-1-yl]-4-hydroxy-3,4-dihydro-2H-1-benzopyran-2-carboxamide;
N-[(2S)-4-{[(2R,4R)-6-chloro-4-hydroxy-3,4-dihydro-2H-1-benzopyran-2-carbonyl]amino}-2-hydroxybicyclo[2.2.2]octan-1-yl]-4-methyl-1,3-thiazole-2-carboxamide;
N-(4-{[(2R)-6-chloro-4-oxo-3,4-dihydro-2H-1-benzopyran-2-carbonyl]amino}bicyclo[2.2.2]octan-1-yl)-5-(trifluoromethoxy)pyridine-2-carboxamide;
N-(4-{[(2S)-6-chloro-4-oxo-3,4-dihydro-2H-1-benzopyran-2-carbonyl]amino}bicyclo[2.2.2]octan-1-yl)-5-(trifluoromethoxy)pyridine-2-carboxamide;
(2R,4R)-6,7-difluoro-N-{3-[2-(4-fluoro-3-methylphenoxy)acetamido]bicyclo[1.1.1]pentan-1-yl}-4-hydroxy-3,4-dihydro-2H-1-benzopyran-2-carboxamide;
(2S,4S)-6-chloro-N-{3-[2-(4-fluoro-3-methylphenoxy)acetamido]bicyclo[1.1.1]pentan-1-yl}-4-hydroxy-3,4-dihydro-2H-1-benzopyran-2-carboxamide;
N-(4-{[(2R,4R)-6-chloro-4-hydroxy-3,4-dihydro-2H-1-benzopyran-2-carbonyl]amino}bicyclo[2.2.2]octan-1-yl)-5-(trifluoromethoxy)pyridine-2-carboxamide;
N-(4-{[(2S,4S)-6-chloro-4-hydroxy-3,4-dihydro-2H-1-benzopyran-2-carbonyl]amino}bicyclo[2.2.2]octan-1-yl)-5-(trifluoromethoxy)pyridine-2-carboxamide;
6-chloro-N-{4-[2-(4-chloro-3-fluorophenoxy)acetamido]-3-oxo-2-azabicyclo[2.2.2]octan-1-yl}-4-oxo-3,4-dihydro-2H-1-benzopyran-2-carboxamide;
rac-(2R,4R)-6-chloro-N-{4-[2-(4-chloro-3-fluorophenoxy)acetamido]-3-oxo-2-azabicyclo[2.2.2]octan-1-yl}-4-hydroxy-3,4-dihydro-2H-1-benzopyran-2-carboxamide;
6-chloro-4-oxo-N-{rac-(1R,2S,4R,5S)-5-[4-(trifluoromethyl)benzamido]bicyclo[2.2.1]heptan-2-yl}-3,4-dihydro-2H-1-benzopyran-2-carboxamide;
(2RS,4RS)-6-chloro-4-hydroxy-N-{(1SR,2RS,4SR,5RS)-5-[4-(trifluoromethyl)benzamido]bicyclo[2.2.1]heptan-2-yl}-3,4-dihydro-2H-1-benzopyran-2-carboxamide;
(2R,4S)-6-chloro-N-{3-[2-(4-chloro-3-fluorophenoxy)acetamido]bicyclo[1.1.1]pentan-1-yl}-7-fluoro-4-hydroxy-3,4-dihydro-2H-1-benzopyran-2-carboxamide;
(2R)-6-chloro-N-{(1RS,2SR,4RS,5SR)-5-[2-(4-chloro-3-fluorophenoxy)acetamido]bicyclo[2.2.1]heptan-2-yl}-4-oxo-3,4-dihydro-2H-1-benzopyran-2-carboxamide;
(2S,4R)-6-chloro-N-{(1RS,2SR,4RS,5SR)-5-[2-(4-chloro-3-fluorophenoxy)acetamido]-7-oxabicyclo[2.2.1]heptan-2-yl}-4-hydroxy-3,4-dihydro-2H-1-benzopyran-2-carboxamide;
(2R,4R)-6-chloro-N-{(1RS,2SR,4RS,5SR)-5-[2-(4-chloro-3-fluorophenoxy)acetamido]-7-oxabicyclo[2.2.1]heptan-2-yl}-4-hydroxy-3,4-dihydro-2H-1-benzopyran-2-carboxamide;
(2R,4R)-6-chloro-N-{(1RS,2SR,4RS,5SR)-5-[2-(4-chloro-3-fluorophenoxy)acetamido]bicyclo[2.2.1]heptan-2-yl}-4-hydroxy-3,4-dihydro-2H-1-benzopyran-2-carboxamide;
(2S,4S)-6-chloro-N-{(1RS,2SR,4RS,5SR)-5-[2-(4-chloro-3-fluorophenoxy)acetamido]-7-oxabicyclo[2.2.1]heptan-2-yl}-4-hydroxy-3,4-dihydro-2H-1-benzopyran-2-carboxamide;
N-(4-{[(2R,4R)-6-chloro-4-hydroxy-3,4-dihydro-2H-1-benzopyran-2-carbonyl]amino}bicyclo[2.1.1]hexan-1-yl)-5-(trifluoromethoxy)pyridine-2-carboxamide;
(2R,4R)-6-chloro-4-hydroxy-N-{4-[4-(trifluoromethyl)benzamido]bicyclo[2.1.1]hexan-1-yl}-3,4-dihydro-2H-1-benzopyran-2-carboxamide;
(2R,4R)-6-chloro-N-{(1S,2R,4S,5R)-5-[2-(4-chloro-3-fluorophenoxy)acetamido]-7-oxabicyclo[2.2.1]heptan-2-yl}-4-hydroxy-3,4-dihydro-2H-1-benzopyran-2-carboxamide;
N-(3-{[(2R,4R)-6-chloro-4-hydroxy-3,4-dihydro-2H-1-benzopyran-2-carbonyl]amino}bicyclo[1.1.1]pentan-1-yl)-5-(trifluoromethoxy)pyridine-2-carboxamide;
N-(3-{[(2R,4R)-6-chloro-4-hydroxy-3,4-dihydro-2H-1-benzopyran-2-carbonyl]amino}bicyclo[1.1.1]pentan-1-yl)-6-(trifluoromethoxy)pyridine-3-carboxamide;
(2R,4R)-6-chloro-N-[3-(4-chloro-3-fluorobenzamido)bicyclo[1.1.1]pentan-1-yl]-4-hydroxy-3,4-dihydro-2H-1-benzopyran-2-carboxamide;
N-(3-{[(2R)-6-chloro-4-oxo-3,4-dihydro-2H-1-benzopyran-2-carbonyl]amino}bicyclo[1.1.1]pentan-1-yl)-5-(trifluoromethoxy)pyridine-2-carboxamide;
N-(3-{[(2R)-6-chloro-4-oxo-3,4-dihydro-2H-1-benzopyran-2-carbonyl]amino}bicyclo[1.1.1]pentan-1-yl)-6-(trifluoromethoxy)pyridine-3-carboxamide;
N-(3-{[(2R,4R)-6-chloro-4-hydroxy-3,4-dihydro-2H-1-benzopyran-2-carbonyl]amino}bicyclo[1.1.1]pentan-1-yl)-1-[cis-3-(trifluoromethoxy)cyclobutyl]-1H-pyrazole-4-carboxamide;
N-(3-{[(2R,4R)-6-chloro-4-hydroxy-3,4-dihydro-2H-1-benzopyran-2-carbonyl]amino}bicyclo[1.1.1]pentan-1-yl)-5-methylpyrazine-2-carboxamide;
N-(3-{[(2R,4R)-6-chloro-4-hydroxy-3,4-dihydro-2H-1-benzopyran-2-carbonyl]amino}bicyclo[1.1.1]pentan-1-yl)-2-cyclopropyl-1,3-oxazole-5-carboxamide;
N-(3-{[(2R,4R)-6-chloro-4-hydroxy-3,4-dihydro-2H-1-benzopyran-2-carbonyl]amino}bicyclo[1.1.1]pentan-1-yl)-2-ethyl-1,3-oxazole-5-carboxamide;
N-(3-{[(2R,4R)-6-chloro-4-hydroxy-3,4-dihydro-2H-1-benzopyran-2-carbonyl]amino}bicyclo[1.1.1]pentan-1-yl)-2-methyl-1,3-thiazole-5-carboxamide;
N-(3-{[(2R,4R)-6-chloro-4-hydroxy-3,4-dihydro-2H-1-benzopyran-2-carbonyl]amino}bicyclo[1.1.1]pentan-1-yl)pyridine-2-carboxamide;
N-(3-{[(2R,4R)-6-chloro-4-hydroxy-3,4-dihydro-2H-1-benzopyran-2-carbonyl]amino}bicyclo[1.1.1]pentan-1-yl)-2-cyclobutyl-1,3-oxazole-5-carboxamide;
N-(3-{[(2R,4R)-6-chloro-4-hydroxy-3,4-dihydro-2H-1-benzopyran-2-carbonyl]amino}bicyclo[1.1.1]pentan-1-yl)-2-[1-(2,2,2-trifluoroethyl)azetidin-3-yl]-1,3-oxazole-5-carboxamide;
2-(azetidin-3-yl)-N-(3-{[(2R,4R)-6-chloro-4-hydroxy-3,4-dihydro-2H-1-benzopyran-2-carbonyl]amino}bicyclo[1.1.1]pentan-1-yl)-1,3-oxazole-5-carboxamide;
N-(3-{[(2R,4R)-6-chloro-4-hydroxy-3,4-dihydro-2H-1-benzopyran-2-carbonyl]amino}bicyclo[1.1.1]pentan-1-yl)-1-phenyl-1H-pyrazole-4-carboxamide;
1-(4-chloro-3-fluorophenyl)-N-(3-{[(2R,4R)-6-chloro-4-hydroxy-3,4-dihydro-2H-1-benzopyran-2-carbonyl]amino}bicyclo[1.1.1]pentan-1-yl)-1H-pyrazole-4-carboxamide;
N-(3-{[(2R,4R)-6-chloro-4-hydroxy-3,4-dihydro-2H-1-benzopyran-2-carbonyl]amino}bicyclo[1.1.1]pentan-1-yl)-1-[6-(trifluoromethyl)pyridin-3-yl]-1H-pyrazole-4-carboxamide;
   and a pharmaceutically acceptable salt, solvate, hydrate, tautomer, N-oxide, or stereoisomer thereof.

In some embodiments, a compound disclosed herein, or a pharmaceutically acceptable salt thereof is formulated as a pharmaceutically acceptable composition comprising a disclosed compound and a pharmaceutically acceptable carrier.

In some embodiments, a compound disclosed herein is selected from a compound set forth in Table 1 or a pharmaceutically acceptable salt, solvate, hydrate, tautomer, N-oxide or stereoisomer thereof.

TABLE 1
Exemplary compounds of the invention.

| Compound | |
|---|---|
| Number | Structure |
| 100 | 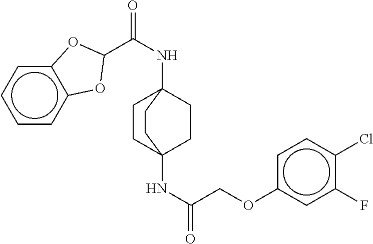 |
| 101 | 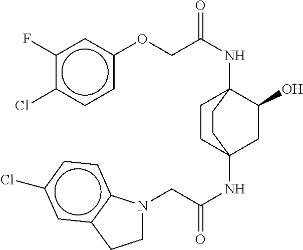 |
| 102 | 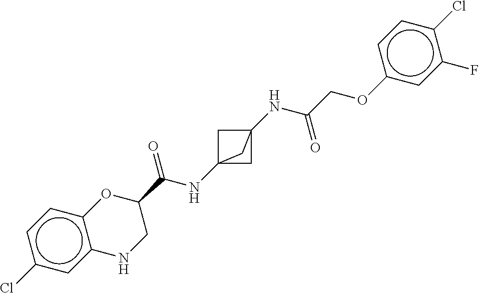 |
| 103 | 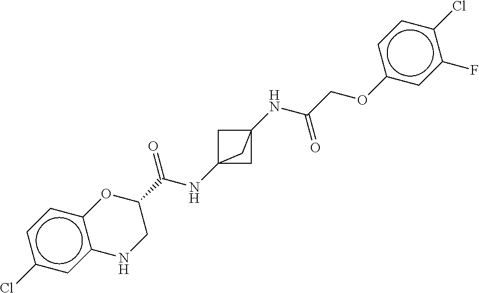 |
| 104 | 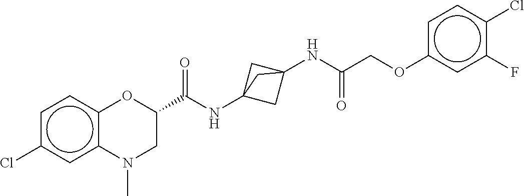 |
| 105 | 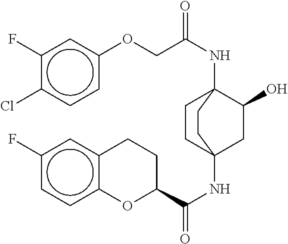 |
| 106 | 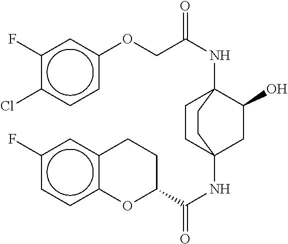 |
| 107 | 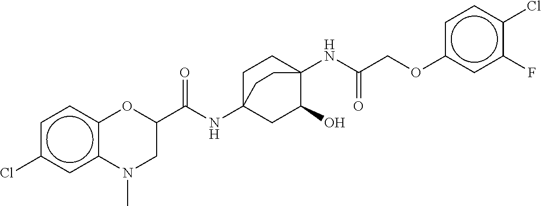 |
| 108 | 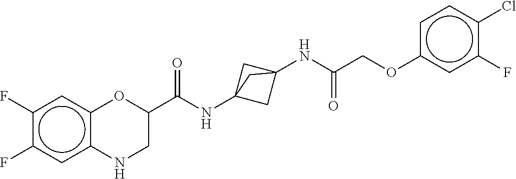 |
| 109 | 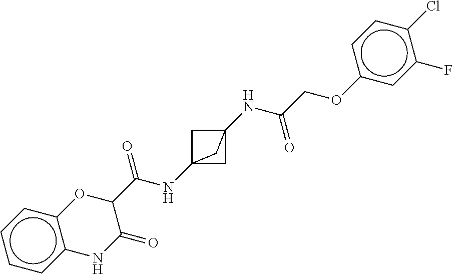 |
| 110 | 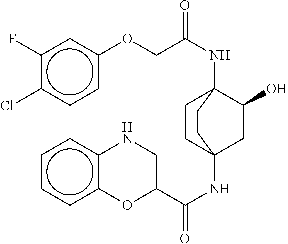 |
| 111 | 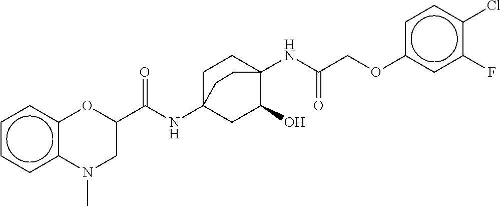 |
| 112 | 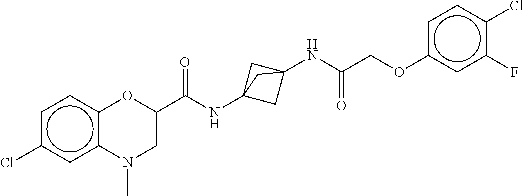 |
| 113 | 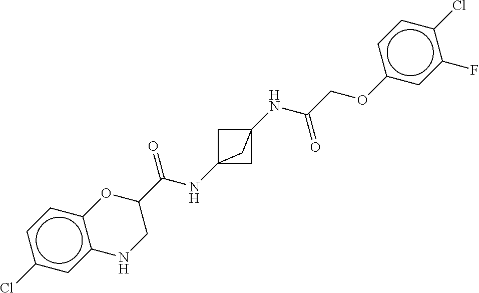 |
| 114 | 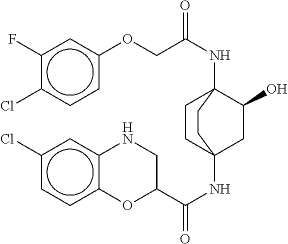 |
| 115 | 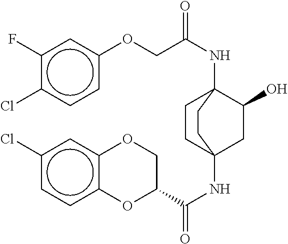 |
| 116 | 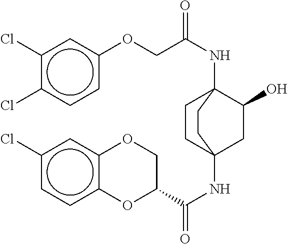 |
| 117 | 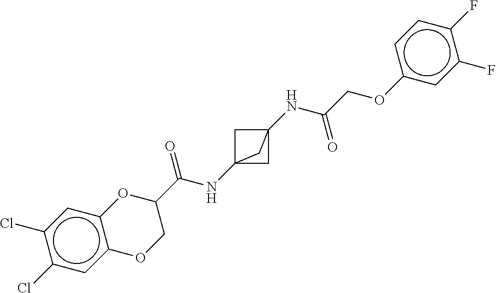 |
| 118 | 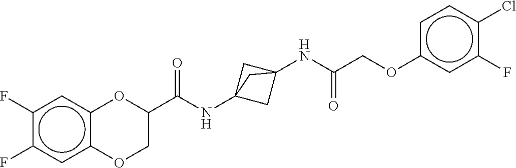 |
| 119 | 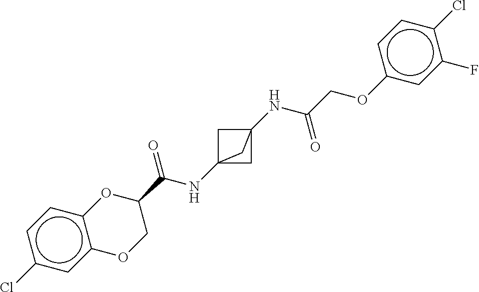 |
| 120 | 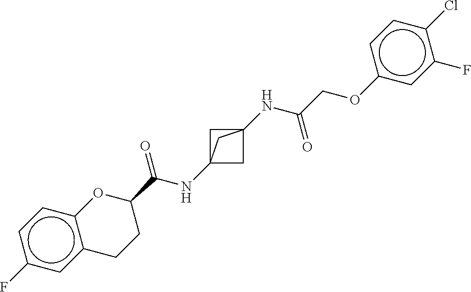 |
| 121 | 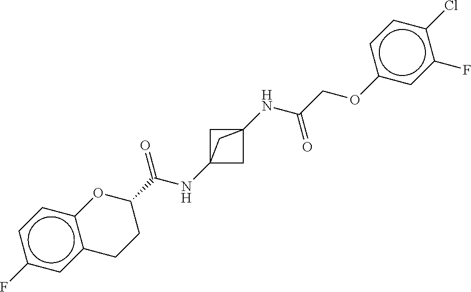 |
| 122 | 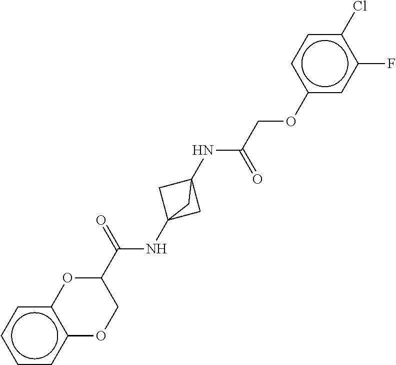 |
| 123 | 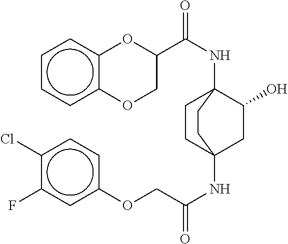 |
| 124 | 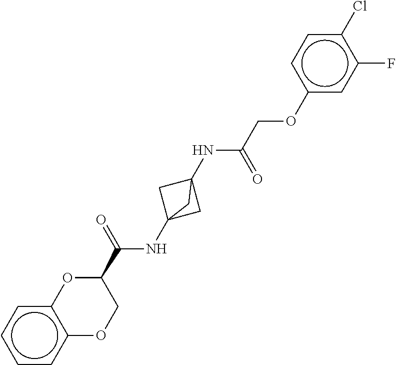 |
| 125 | 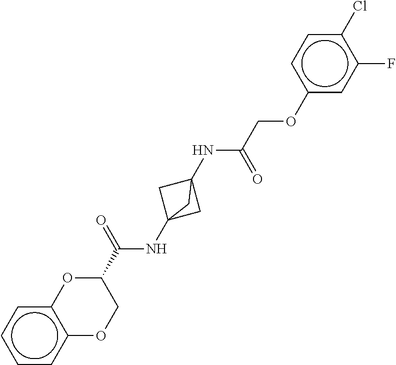 |
| 126 | 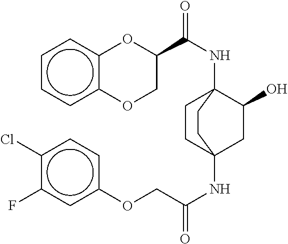 |
| 127 | 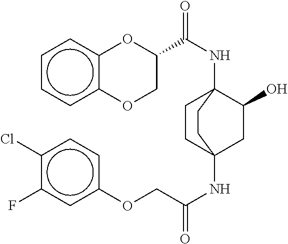 |
| 128 | 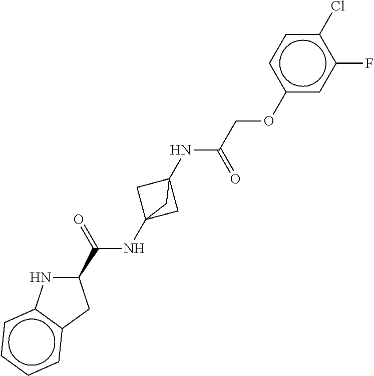 |
| 129 | 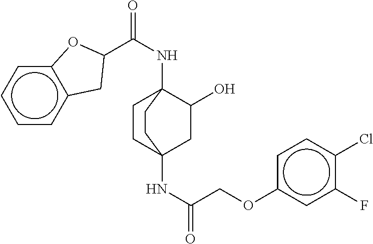 |
| 130 | 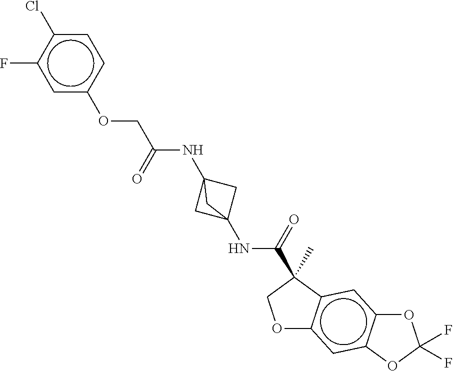 |
| 131 | 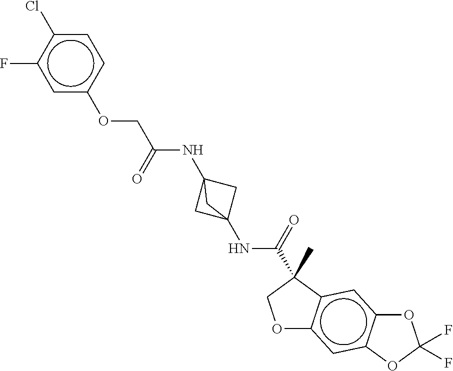 |
| 132 | 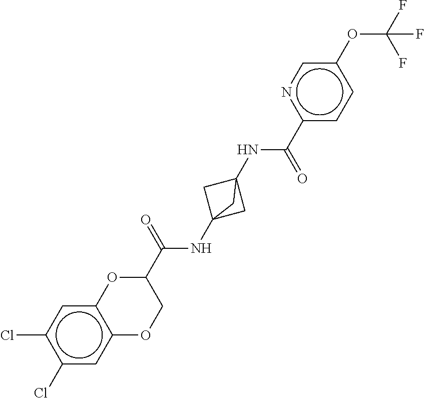 |
| 133 | 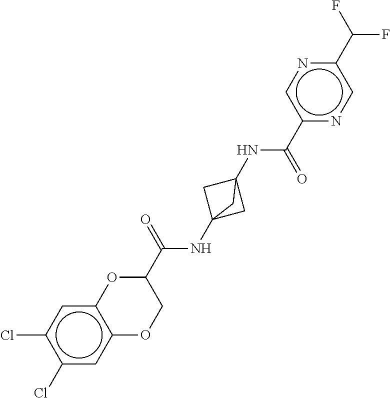 |
| 134 | 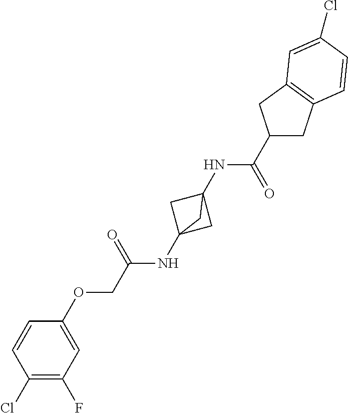 [SMILES: O=C(COc1ccc(Cl)c(F)c1)NC12CC(NC(=O)C3Cc4ccc(Cl)cc4C3)(C1)C2] |
| 135 | 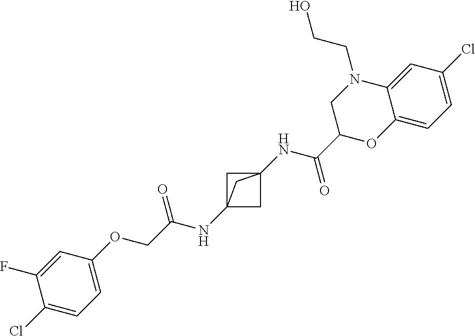 [SMILES: O=C(COc1ccc(Cl)c(F)c1)NC12CC(NC(=O)C3CN(CCO)c4cc(Cl)ccc4O3)(C1)C2] |
| 136 | 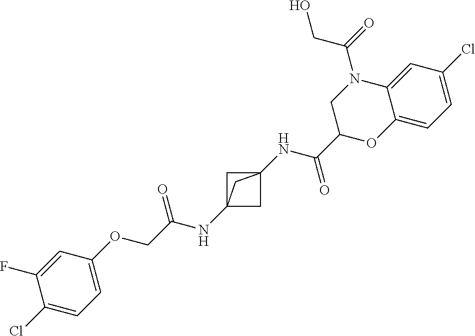 [SMILES: O=C(COc1ccc(Cl)c(F)c1)NC12CC(NC(=O)C3CN(C(=O)CO)c4cc(Cl)ccc4O3)(C1)C2] |
| 137 | 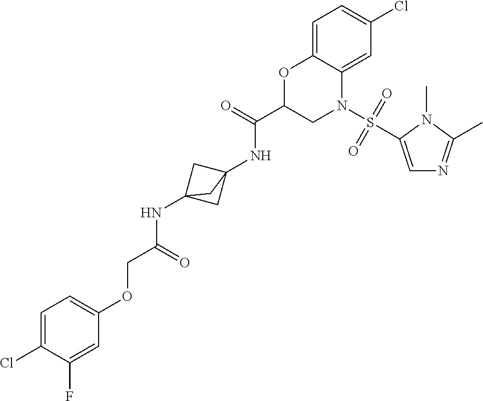 [SMILES: Cc1ncc(S(=O)(=O)N2CC(C(=O)NC34CC(NC(=O)COc5ccc(Cl)c(F)c5)(C3)C4)Oc3ccc(Cl)cc32)n1C] |
| 138 | 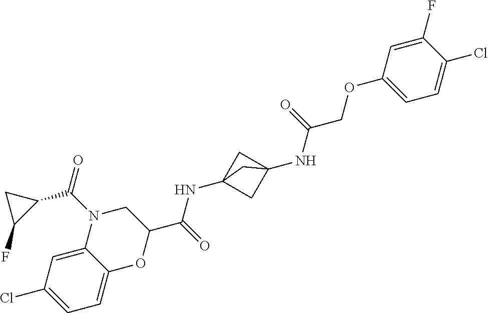 [SMILES: O=C(COc1ccc(Cl)c(F)c1)NC12CC(NC(=O)C3CN(C(=O)[C@H]4C[C@@H]4F)c4cc(Cl)ccc4O3)(C1)C2] |
| 139 | 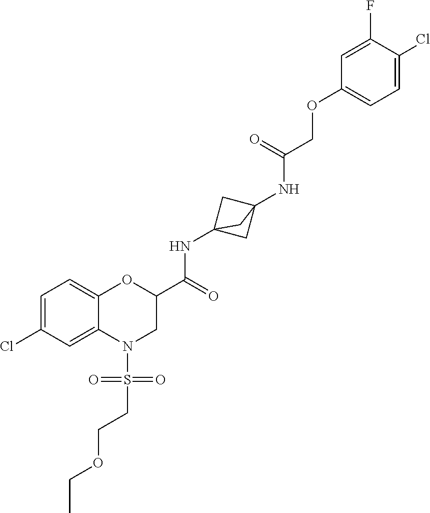 [SMILES: CCOCCS(=O)(=O)N1CC(C(=O)NC23CC(NC(=O)COc4ccc(Cl)c(F)c4)(C2)C3)Oc2ccc(Cl)cc21] |
| 140 | 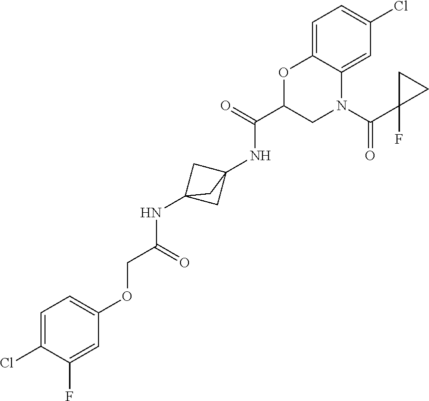 [SMILES: O=C(COc1ccc(Cl)c(F)c1)NC12CC(NC(=O)C3CN(C(=O)C4(F)CC4)c4cc(Cl)ccc4O3)(C1)C2] |
| 141 | 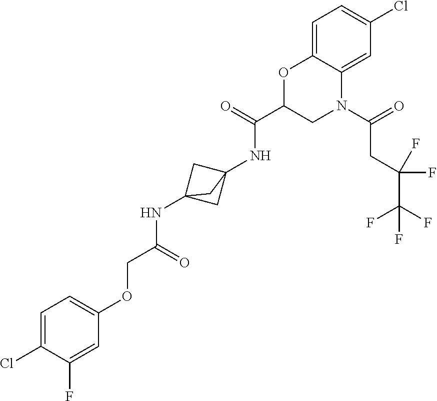 [SMILES: O=C(COc1ccc(Cl)c(F)c1)NC12CC(NC(=O)C3CN(C(=O)CC(F)(F)C(F)(F)F)c4cc(Cl)ccc4O3)(C1)C2] |
| 142 | 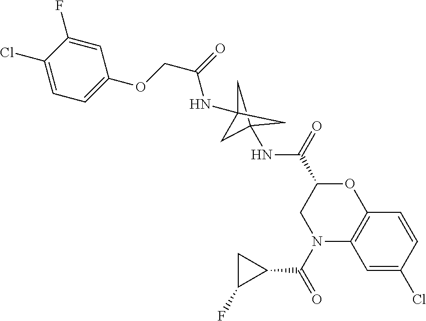 [SMILES: O=C(COc1ccc(Cl)c(F)c1)NC12CC(NC(=O)[C@H]3CN(C(=O)[C@H]4C[C@H]4F)c4cc(Cl)ccc4O3)(C1)C2] |
| 143 | 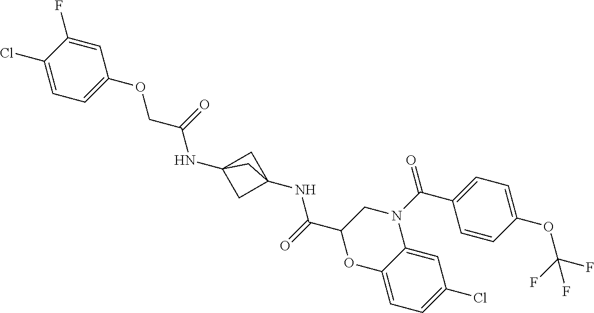 [SMILES: O=C(COc1ccc(Cl)c(F)c1)NC12CC(NC(=O)C3CN(C(=O)c4ccc(OC(F)(F)F)cc4)c4cc(Cl)ccc4O3)(C1)C2] |
| 144 | 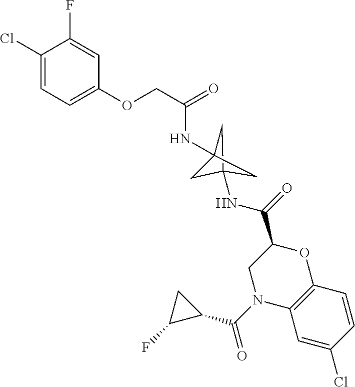 [SMILES: O=C(COc1ccc(Cl)c(F)c1)NC12CC(NC(=O)[C@@H]3CN(C(=O)[C@H]4C[C@H]4F)c4cc(Cl)ccc4O3)(C1)C2] |
| 145 | 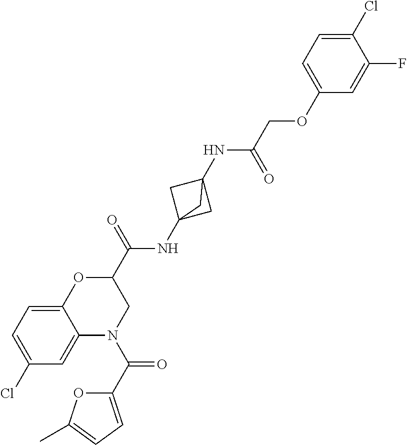 [SMILES: Cc1ccc(C(=O)N2CC(C(=O)NC34CC(NC(=O)COc5ccc(Cl)c(F)c5)(C3)C4)Oc3ccc(Cl)cc32)o1] |
| 146 | 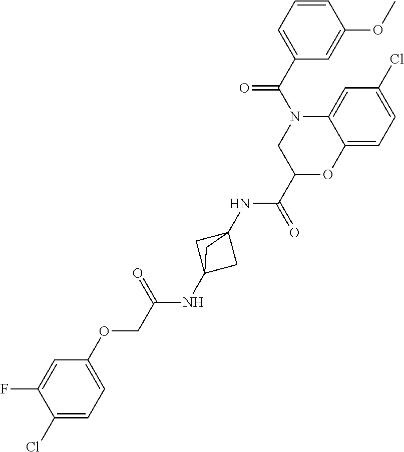 [SMILES: COc1cccc(C(=O)N2CC(C(=O)NC34CC(NC(=O)COc5ccc(Cl)c(F)c5)(C3)C4)Oc3ccc(Cl)cc32)c1] |
| 147 | 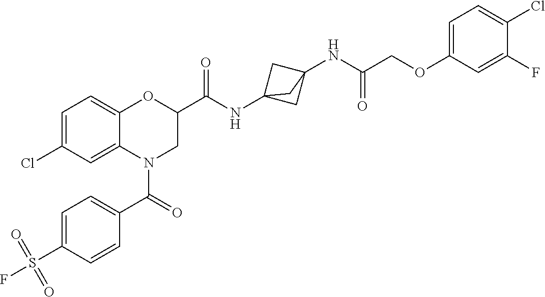 [SMILES: O=C(COc1ccc(Cl)c(F)c1)NC12CC(NC(=O)C3CN(C(=O)c4ccc(S(=O)(=O)F)cc4)c4cc(Cl)ccc4O3)(C1)C2] |
| 148 | 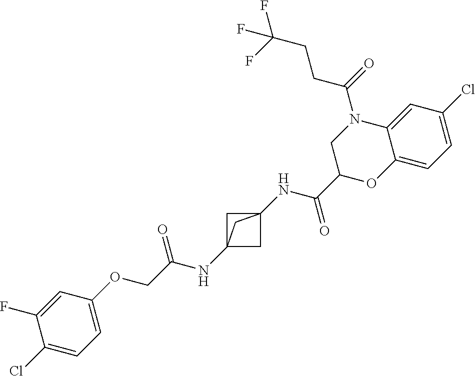 [SMILES: O=C(COc1ccc(Cl)c(F)c1)NC12CC(NC(=O)C3CN(C(=O)CCC(F)(F)F)c4cc(Cl)ccc4O3)(C1)C2] |
| 149 | 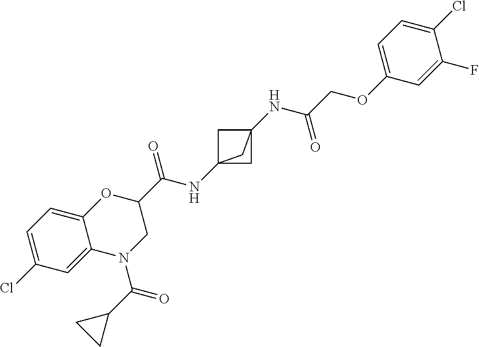 [SMILES: O=C(COc1ccc(Cl)c(F)c1)NC12CC(NC(=O)C3CN(C(=O)C4CC4)c4cc(Cl)ccc4O3)(C1)C2] |
| 150 | 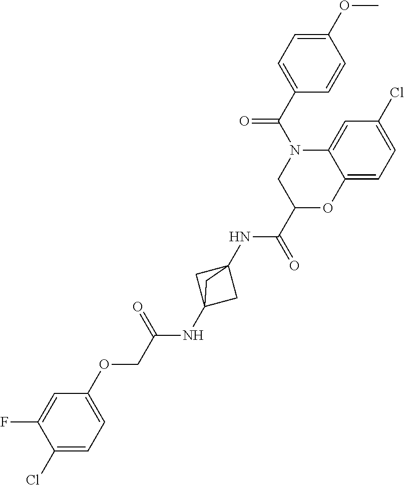 [SMILES: COc1ccc(C(=O)N2CC(C(=O)NC34CC(NC(=O)COc5ccc(Cl)c(F)c5)(C3)C4)Oc3ccc(Cl)cc32)cc1] |
| 151 | 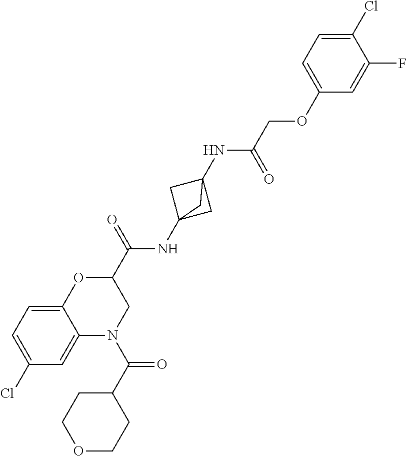 [SMILES: O=C(COc1ccc(Cl)c(F)c1)NC12CC(NC(=O)C3CN(C(=O)C4CCOCC4)c4cc(Cl)ccc4O3)(C1)C2] |
| 152 | 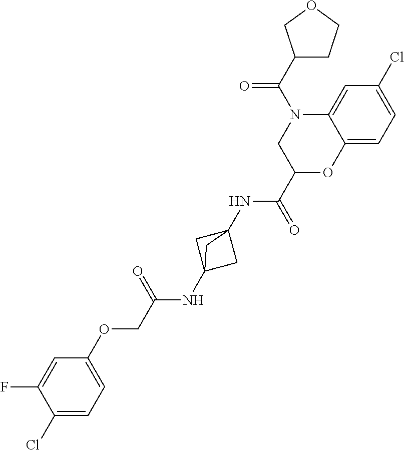 [SMILES: O=C(COc1ccc(Cl)c(F)c1)NC12CC(NC(=O)C3CN(C(=O)C4CCOC4)c4cc(Cl)ccc4O3)(C1)C2] |
| 153 | 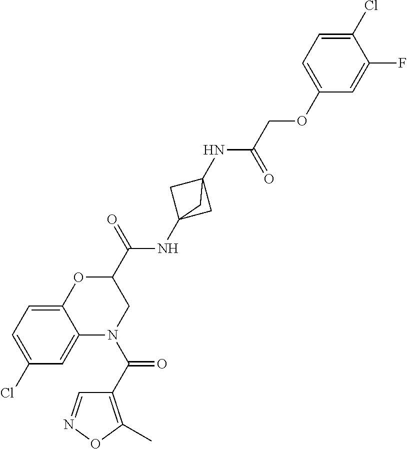 [SMILES: Cc1oncc1C(=O)N1CC(C(=O)NC23CC(NC(=O)COc4ccc(Cl)c(F)c4)(C2)C3)Oc2ccc(Cl)cc21] |
| 154 | 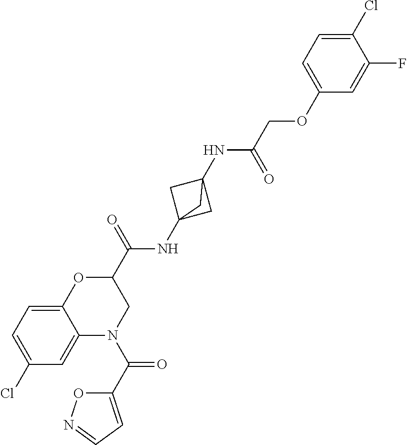 [SMILES: O=C(COc1ccc(Cl)c(F)c1)NC12CC(NC(=O)C3CN(C(=O)c4ccno4)c4cc(Cl)ccc4O3)(C1)C2] |
| 155 | 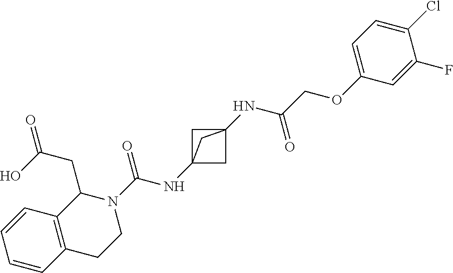 [SMILES: O=C(O)CC1c2ccccc2CCN1C(=O)NC12CC(NC(=O)COc3ccc(Cl)c(F)c3)(C1)C2] |
| 156 | 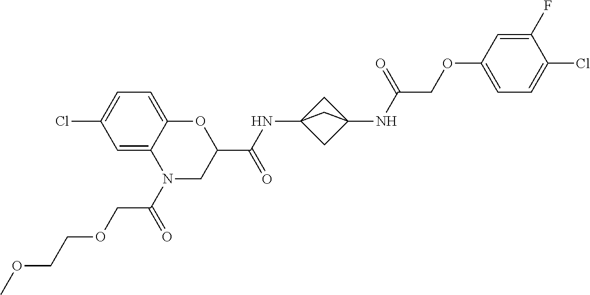 [SMILES: COCCOCC(=O)N1CC(C(=O)NC23CC(NC(=O)COc4ccc(Cl)c(F)c4)(C2)C3)Oc2ccc(Cl)cc21] |
| 157 | 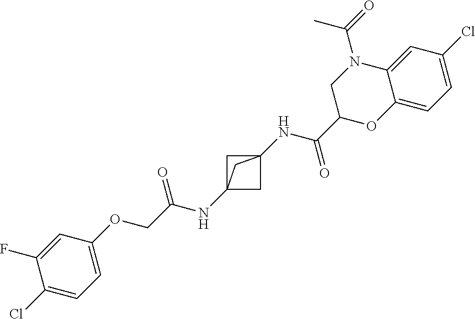 [SMILES: CC(=O)N1CC(C(=O)NC23CC(NC(=O)COc4ccc(Cl)c(F)c4)(C2)C3)Oc2ccc(Cl)cc21] |
| 158 | 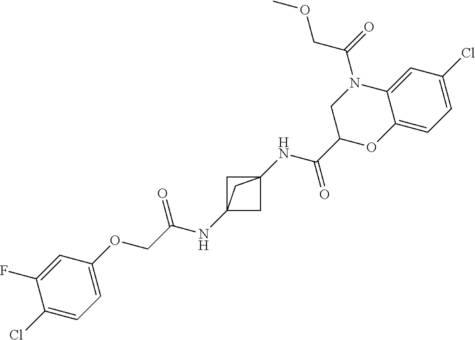 [SMILES: COCC(=O)N1CC(C(=O)NC23CC(NC(=O)COc4ccc(Cl)c(F)c4)(C2)C3)Oc2ccc(Cl)cc21] |
| 159 | 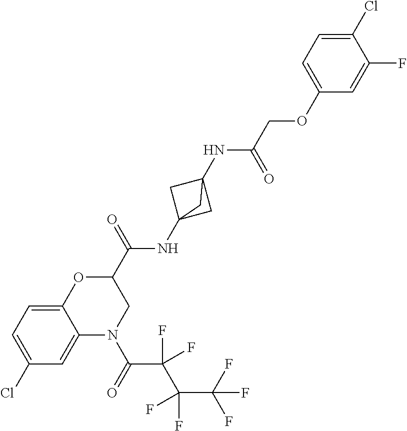 [SMILES: O=C(COc1ccc(Cl)c(F)c1)NC12CC(NC(=O)C3CN(C(=O)C(F)(F)C(F)(F)C(F)(F)F)c4cc(Cl)ccc4O3)(C1)C2] |
| 160 | 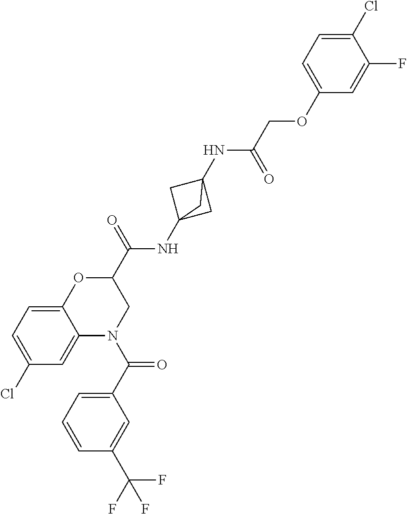 [SMILES: O=C(COc1ccc(Cl)c(F)c1)NC12CC(NC(=O)C3CN(C(=O)c4cccc(C(F)(F)F)c4)c4cc(Cl)ccc4O3)(C1)C2] |
| 161 | 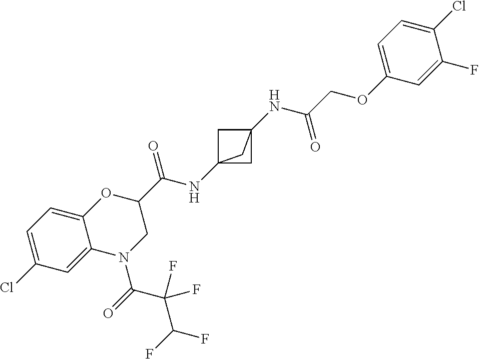 [SMILES: O=C(COc1ccc(Cl)c(F)c1)NC12CC(NC(=O)C3CN(C(=O)C(F)(F)C(F)F)c4cc(Cl)ccc4O3)(C1)C2] |
| 162 | 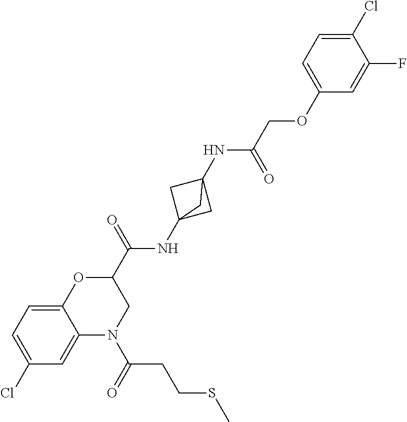 [SMILES: CSCCC(=O)N1CC(C(=O)NC23CC(NC(=O)COc4ccc(Cl)c(F)c4)(C2)C3)Oc2ccc(Cl)cc21] |
| 163 | 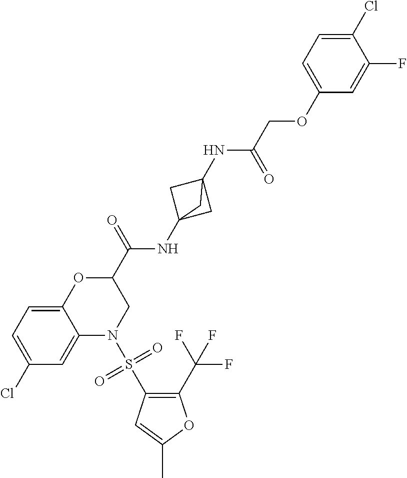 [SMILES: Cc1cc(S(=O)(=O)N2CC(C(=O)NC34CC(NC(=O)COc5ccc(Cl)c(F)c5)(C3)C4)Oc3ccc(Cl)cc32)c(C(F)(F)F)o1] |
| 164 | 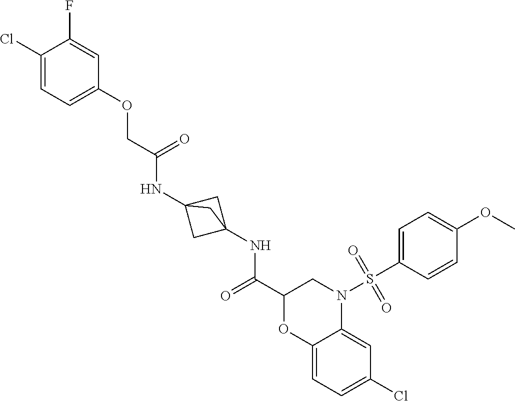 [SMILES: COc1ccc(S(=O)(=O)N2CC(C(=O)NC34CC(NC(=O)COc5ccc(Cl)c(F)c5)(C3)C4)Oc3ccc(Cl)cc32)cc1] |
| 165 | 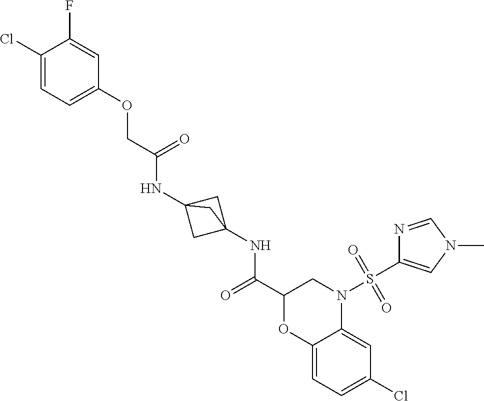 [SMILES: Cn1cnc(S(=O)(=O)N2CC(C(=O)NC34CC(NC(=O)COc5ccc(Cl)c(F)c5)(C3)C4)Oc3ccc(Cl)cc32)c1] |
| 166 | 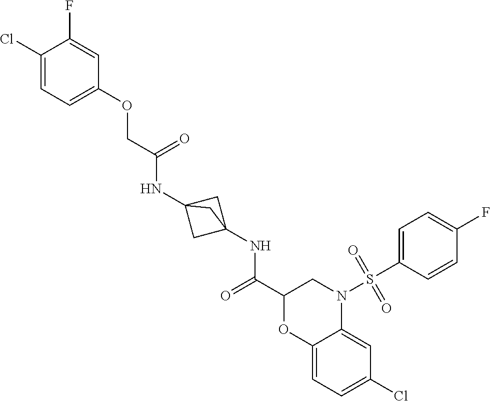 [SMILES: O=C(COc1ccc(Cl)c(F)c1)NC12CC(NC(=O)C3CN(S(=O)(=O)c4ccc(F)cc4)c4cc(Cl)ccc4O3)(C1)C2] |
| 167 | 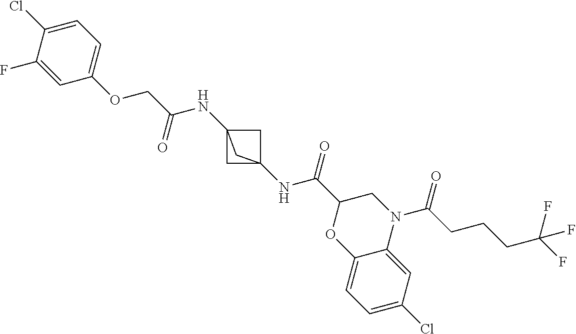 [SMILES: O=C(COc1ccc(Cl)c(F)c1)NC12CC(NC(=O)C3CN(C(=O)CCCC(F)(F)F)c4cc(Cl)ccc4O3)(C1)C2] |
| 168 | 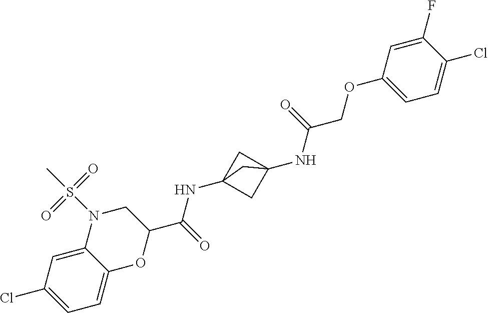 [SMILES: CS(=O)(=O)N1CC(C(=O)NC23CC(NC(=O)COc4ccc(Cl)c(F)c4)(C2)C3)Oc2ccc(Cl)cc21] |
| 169 | 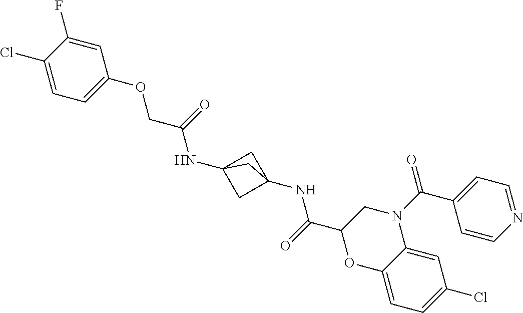 [SMILES: O=C(COc1ccc(Cl)c(F)c1)NC12CC(NC(=O)C3CN(C(=O)c4ccncc4)c4cc(Cl)ccc4O3)(C1)C2] |
| 170 | 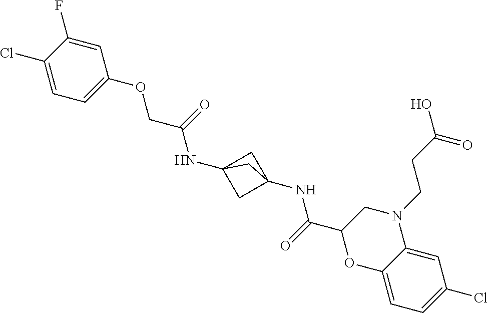 [SMILES: O=C(O)CCN1CC(C(=O)NC23CC(NC(=O)COc4ccc(Cl)c(F)c4)(C2)C3)Oc2ccc(Cl)cc21] |
| 171 | 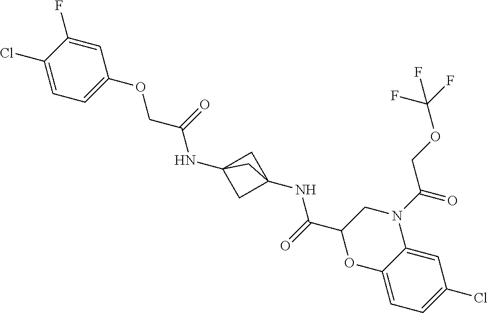 [SMILES: O=C(COc1ccc(Cl)c(F)c1)NC12CC(NC(=O)C3CN(C(=O)COC(F)(F)F)c4cc(Cl)ccc4O3)(C1)C2] |
| 172 | 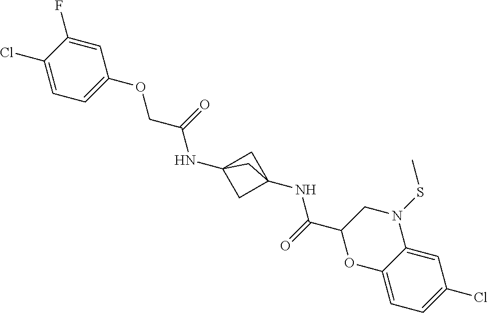 [SMILES: CSN1CC(C(=O)NC23CC(NC(=O)COc4ccc(Cl)c(F)c4)(C2)C3)Oc2ccc(Cl)cc21] |
| 173 | 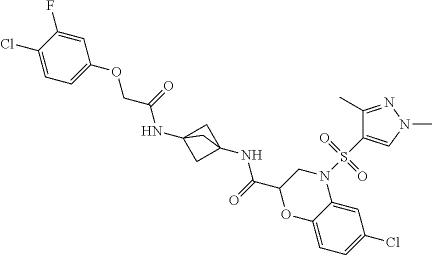 [SMILES: Cc1nn(C)cc1S(=O)(=O)N1CC(C(=O)NC23CC(NC(=O)COc4ccc(Cl)c(F)c4)(C2)C3)Oc2ccc(Cl)cc21] |
| 174 | 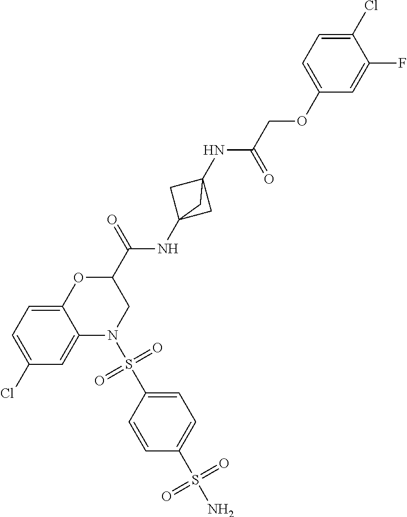 [SMILES: NS(=O)(=O)c1ccc(S(=O)(=O)N2CC(C(=O)NC34CC(NC(=O)COc5ccc(Cl)c(F)c5)(C3)C4)Oc3ccc(Cl)cc32)cc1] |
| 175 | 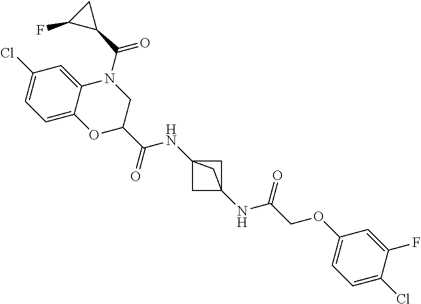 [SMILES: O=C(COc1ccc(Cl)c(F)c1)NC12CC(NC(=O)C3CN(C(=O)[C@@H]4C[C@@H]4F)c4cc(Cl)ccc4O3)(C1)C2] |
| 176 | 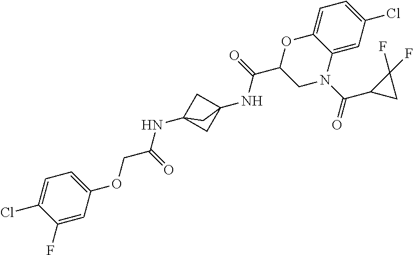 [SMILES: O=C(COc1ccc(Cl)c(F)c1)NC12CC(NC(=O)C3CN(C(=O)C4CC4(F)F)c4cc(Cl)ccc4O3)(C1)C2] |
| 177 | 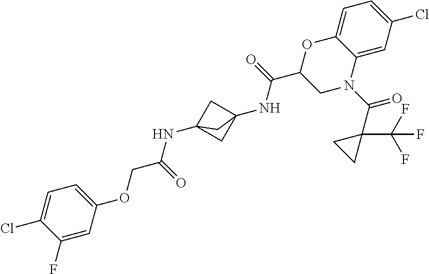 [SMILES: O=C(COc1ccc(Cl)c(F)c1)NC12CC(NC(=O)C3CN(C(=O)C4(C(F)(F)F)CC4)c4cc(Cl)ccc4O3)(C1)C2] |
| 178 | 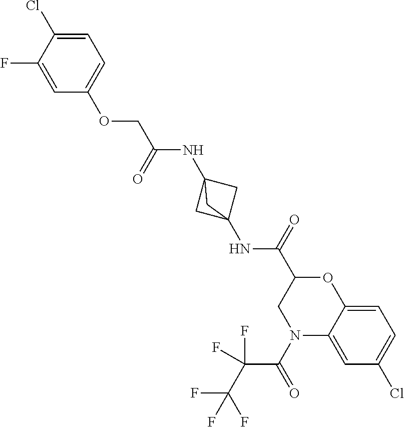 [SMILES: O=C(COc1ccc(Cl)c(F)c1)NC12CC(NC(=O)C3CN(C(=O)C(F)(F)C(F)(F)F)c4cc(Cl)ccc4O3)(C1)C2] |
| 179 | 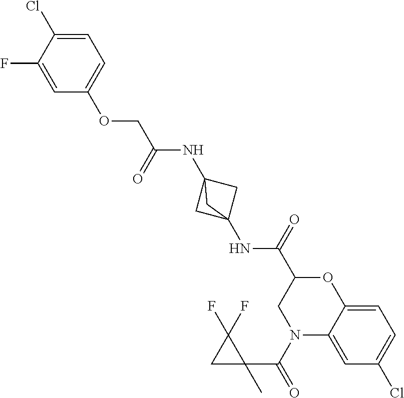 [SMILES: CC1(C(=O)N2CC(C(=O)NC34CC(NC(=O)COc5ccc(Cl)c(F)c5)(C3)C4)Oc3ccc(Cl)cc32)CC1(F)F] |
| 180 | 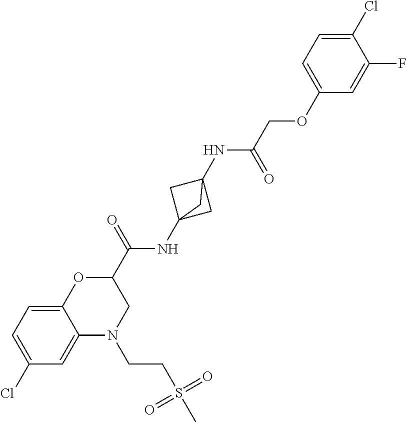 [SMILES: CS(=O)(=O)CCN1CC(C(=O)NC23CC(NC(=O)COc4ccc(Cl)c(F)c4)(C2)C3)Oc2ccc(Cl)cc21] |
| 181 | 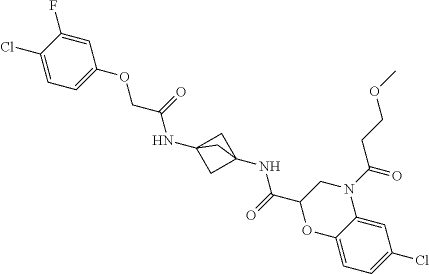 [SMILES: COCCC(=O)N1CC(C(=O)NC23CC(NC(=O)COc4ccc(Cl)c(F)c4)(C2)C3)Oc2ccc(Cl)cc21] |
| 182 | 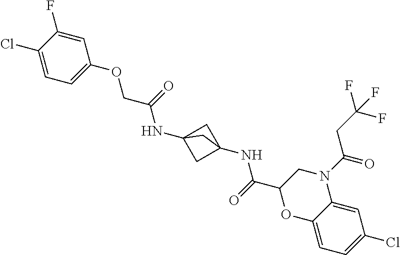 [SMILES: O=C(COc1ccc(Cl)c(F)c1)NC12CC(NC(=O)C3CN(C(=O)CC(F)(F)F)c4cc(Cl)ccc4O3)(C1)C2] |
| 183 | 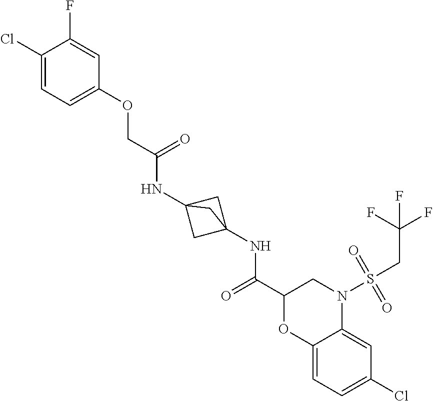 [SMILES: O=C(COc1ccc(Cl)c(F)c1)NC12CC(NC(=O)C3CN(S(=O)(=O)CC(F)(F)F)c4cc(Cl)ccc4O3)(C1)C2] |
| 184 | 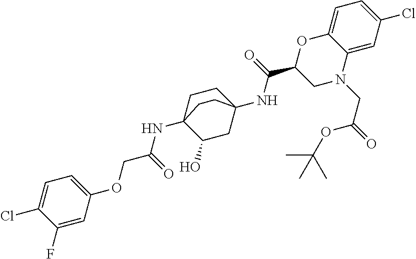 [SMILES: CC(C)(C)OC(=O)CN1C[C@@H](C(=O)NC23CCC(NC(=O)COc4ccc(Cl)c(F)c4)(CC2)[C@@H](O)C3)Oc2ccc(Cl)cc21] |
| 185 | 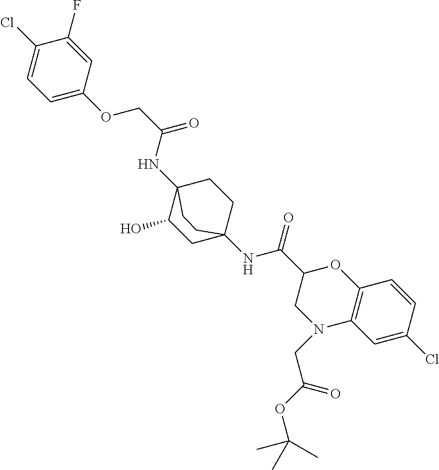 [SMILES: CC(C)(C)OC(=O)CN1CC(C(=O)NC23CCC(NC(=O)COc4ccc(Cl)c(F)c4)(CC2)[C@@H](O)C3)Oc2ccc(Cl)cc21] |
| 186 | 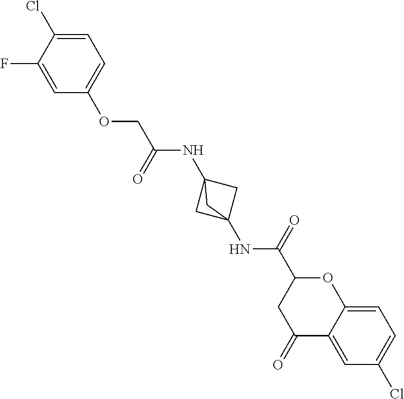 [SMILES: O=C(COc1ccc(Cl)c(F)c1)NC12CC(NC(=O)C3CC(=O)c4cc(Cl)ccc4O3)(C1)C2] |
| 187 | 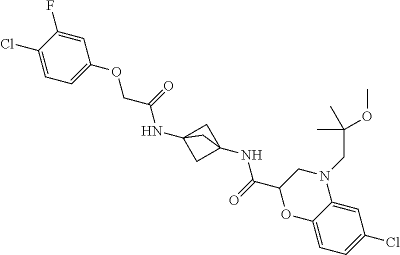 [SMILES: COC(C)(C)CN1CC(C(=O)NC23CC(NC(=O)COc4ccc(Cl)c(F)c4)(C2)C3)Oc2ccc(Cl)cc21] |
| 188 | 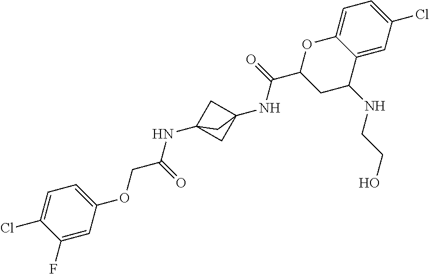 [SMILES: O=C(COc1ccc(Cl)c(F)c1)NC12CC(NC(=O)C3CC(NCCO)c4cc(Cl)ccc4O3)(C1)C2] |
| 189 | 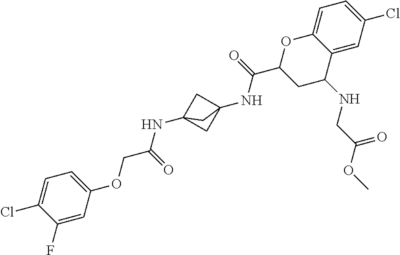 [SMILES: COC(=O)CNC1CC(C(=O)NC23CC(NC(=O)COc4ccc(Cl)c(F)c4)(C2)C3)Oc2ccc(Cl)cc21] |
| 190 | 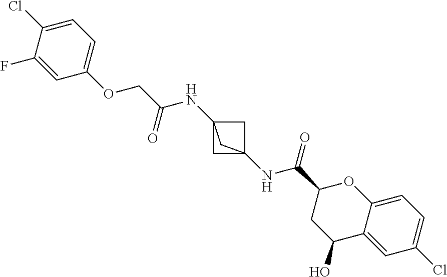 [SMILES: O=C(COc1ccc(Cl)c(F)c1)NC12CC(NC(=O)[C@@H]3C[C@H](O)c4cc(Cl)ccc4O3)(C1)C2] |
| 191 | 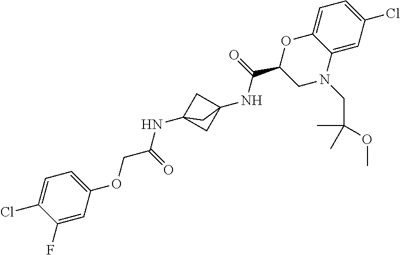 [SMILES: COC(C)(C)CN1C[C@@H](C(=O)NC23CC(NC(=O)COc4ccc(Cl)c(F)c4)(C2)C3)Oc2ccc(Cl)cc21] |
| 192 | 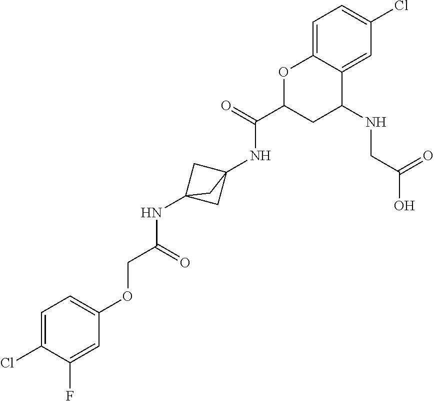 [SMILES: O=C(O)CNC1CC(C(=O)NC23CC(NC(=O)COc4ccc(Cl)c(F)c4)(C2)C3)Oc2ccc(Cl)cc21] |
| 193 | 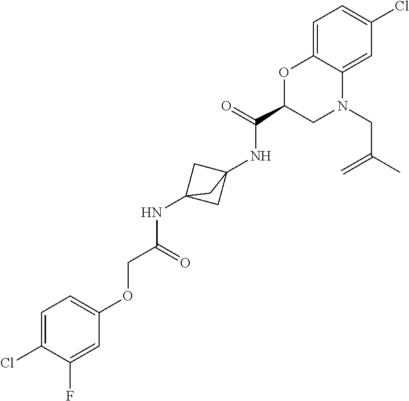 [SMILES: C=C(C)CN1C[C@@H](C(=O)NC23CC(NC(=O)COc4ccc(Cl)c(F)c4)(C2)C3)Oc2ccc(Cl)cc21] |
| 194 | 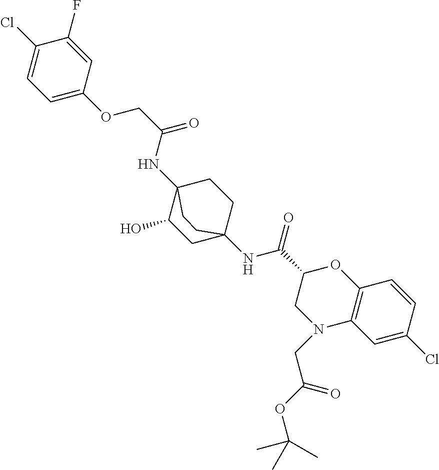 [SMILES: CC(C)(C)OC(=O)CN1C[C@H](C(=O)NC23CCC(NC(=O)COc4ccc(Cl)c(F)c4)(CC2)[C@@H](O)C3)Oc2ccc(Cl)cc21] |
| 195 | 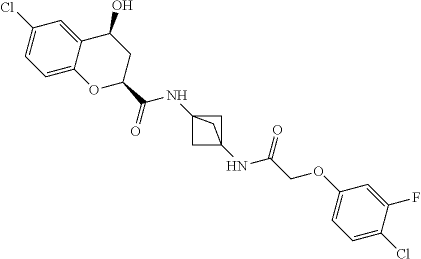 [SMILES: O=C(COc1ccc(Cl)c(F)c1)NC12CC(NC(=O)[C@@H]3C[C@H](O)c4cc(Cl)ccc4O3)(C1)C2] |
| 196 | 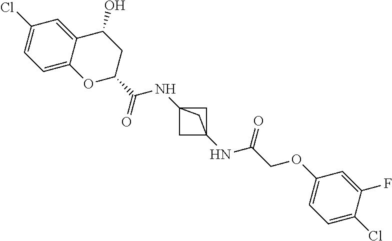 [SMILES: O=C(COc1ccc(Cl)c(F)c1)NC12CC(NC(=O)[C@H]3C[C@@H](O)c4cc(Cl)ccc4O3)(C1)C2] |
| 197 | 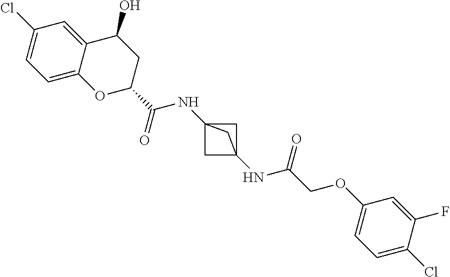 [SMILES: O=C(COc1ccc(Cl)c(F)c1)NC12CC(NC(=O)[C@H]3C[C@H](O)c4cc(Cl)ccc4O3)(C1)C2] |
| 198 | 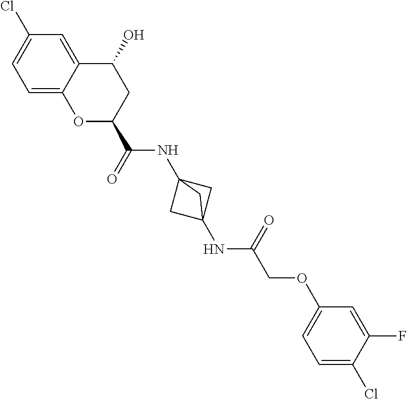 [SMILES: O=C(COc1ccc(Cl)c(F)c1)NC12CC(NC(=O)[C@@H]3C[C@@H](O)c4cc(Cl)ccc4O3)(C1)C2] |
| 199 | 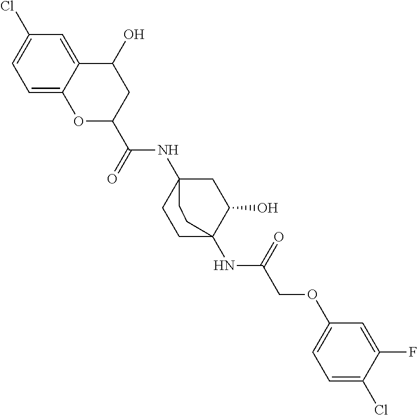 [SMILES: O=C(COc1ccc(Cl)c(F)c1)NC12CCC(NC(=O)C3CC(O)c4cc(Cl)ccc4O3)(CC1)C[C@@H]2O] |
| 199C | 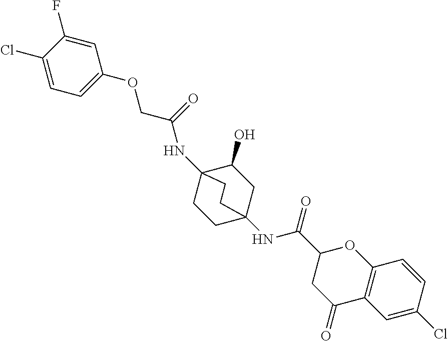 [SMILES: O=C(COc1ccc(Cl)c(F)c1)NC12CCC(NC(=O)C3CC(=O)c4cc(Cl)ccc4O3)(CC1)C[C@@H]2O] |
| 200 | 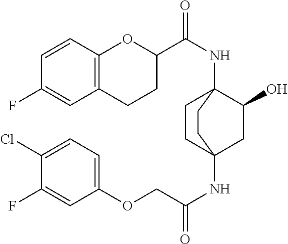 [SMILES: O=C(COc1ccc(Cl)c(F)c1)NC12CCC(NC(=O)C3CCc4cc(F)ccc4O3)(CC1)[C@@H](O)C2] |
| 201 | 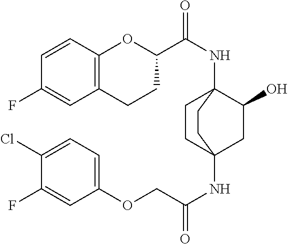 [SMILES: O=C(COc1ccc(Cl)c(F)c1)NC12CCC(NC(=O)[C@@H]3CCc4cc(F)ccc4O3)(CC1)[C@@H](O)C2] |
| 202 | 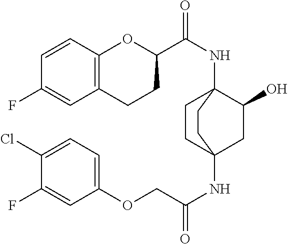 [SMILES: O=C(COc1ccc(Cl)c(F)c1)NC12CCC(NC(=O)[C@H]3CCc4cc(F)ccc4O3)(CC1)[C@@H](O)C2] |
| 203 | 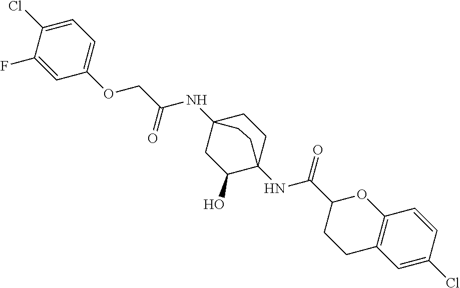 [SMILES: O=C(COc1ccc(Cl)c(F)c1)NC12CCC(NC(=O)C3CCc4cc(Cl)ccc4O3)(CC1)[C@@H](O)C2] |
| 204 | 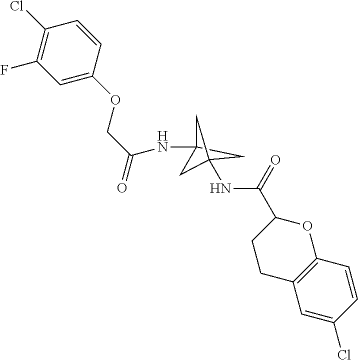 [SMILES: O=C(COc1ccc(Cl)c(F)c1)NC12CC(NC(=O)C3CCc4cc(Cl)ccc4O3)(C1)C2] |
| 205 | 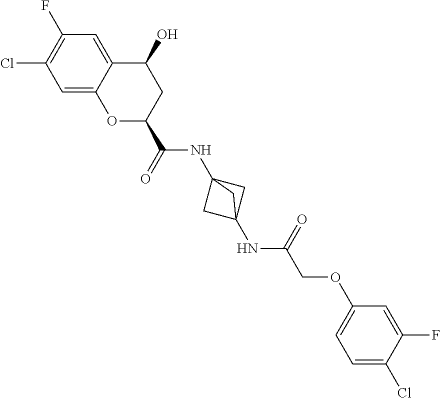 [SMILES: O=C(COc1ccc(Cl)c(F)c1)NC12CC(NC(=O)[C@@H]3C[C@H](O)c4cc(F)c(Cl)cc4O3)(C1)C2] |
| 206 | 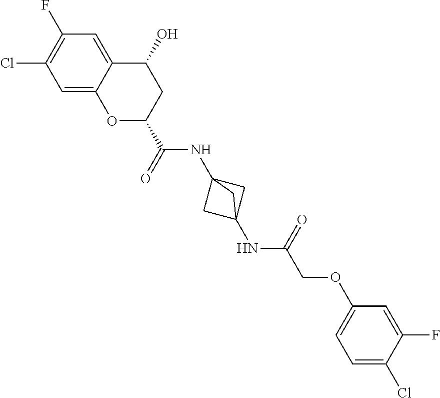 [SMILES: O=C(COc1ccc(Cl)c(F)c1)NC12CC(NC(=O)[C@H]3C[C@@H](O)c4cc(F)c(Cl)cc4O3)(C1)C2] |
| 207 | 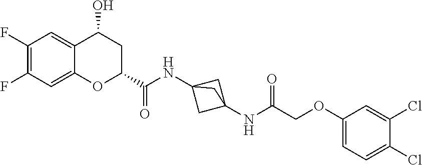 [SMILES: O=C(COc1ccc(Cl)c(Cl)c1)NC12CC(NC(=O)[C@H]3C[C@@H](O)c4cc(F)c(F)cc4O3)(C1)C2] |
| 207E | 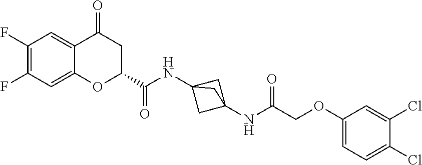 [SMILES: O=C(COc1ccc(Cl)c(Cl)c1)NC12CC(NC(=O)[C@H]3CC(=O)c4cc(F)c(F)cc4O3)(C1)C2] |
| 208 | 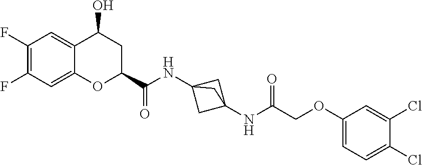 [SMILES: O=C(COc1ccc(Cl)c(Cl)c1)NC12CC(NC(=O)[C@@H]3C[C@H](O)c4cc(F)c(F)cc4O3)(C1)C2] |
| 209 | 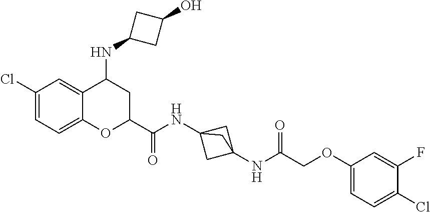 [SMILES: O=C(COc1ccc(Cl)c(F)c1)NC12CC(NC(=O)C3CC(N[C@H]4C[C@@H](O)C4)c4cc(Cl)ccc4O3)(C1)C2] |
| 210 | 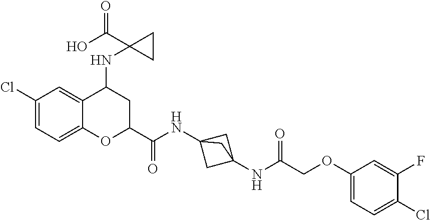 [SMILES: O=C(COc1ccc(Cl)c(F)c1)NC12CC(NC(=O)C3CC(NC4(C(=O)O)CC4)c4cc(Cl)ccc4O3)(C1)C2] |
| 211 | 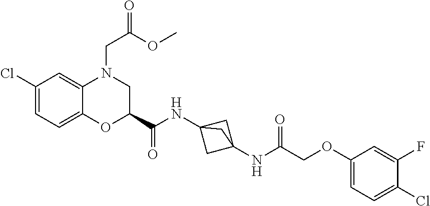 [SMILES: COC(=O)CN1C[C@@H](C(=O)NC23CC(NC(=O)COc4ccc(Cl)c(F)c4)(C2)C3)Oc2ccc(Cl)cc21] |
| 212 | 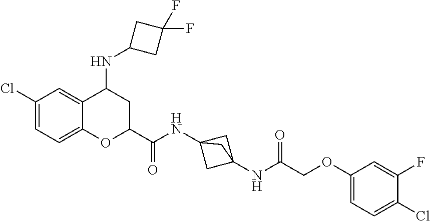 [SMILES: O=C(COc1ccc(Cl)c(F)c1)NC12CC(NC(=O)C3CC(NC4CC(F)(F)C4)c4cc(Cl)ccc4O3)(C1)C2] |
| 213 | 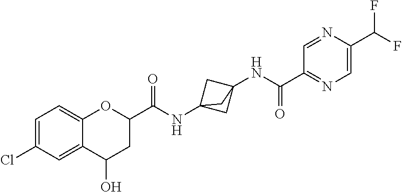 [SMILES: O=C(NC12CC(NC(=O)C3CC(O)c4cc(Cl)ccc4O3)(C1)C2)c1cnc(C(F)F)cn1] |
| 214 | 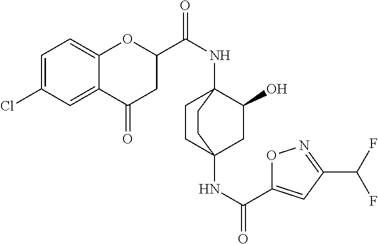 [SMILES: O=C(NC12CCC(NC(=O)C3CC(=O)c4cc(Cl)ccc4O3)(CC1)[C@@H](O)C2)c1cc(C(F)F)no1] |
| 215 | 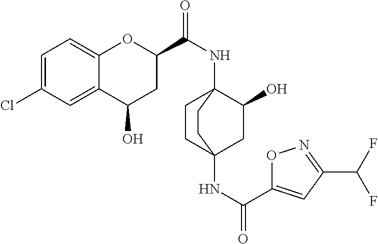 [SMILES: O=C(NC12CCC(NC(=O)[C@H]3C[C@@H](O)c4cc(Cl)ccc4O3)(CC1)[C@@H](O)C2)c1cc(C(F)F)no1] |
| 216 | 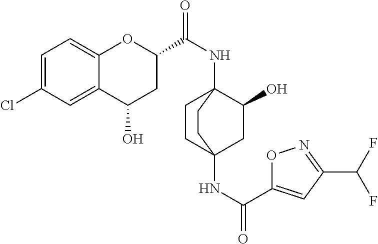 [SMILES: O=C(NC12CCC(NC(=O)[C@@H]3C[C@H](O)c4cc(Cl)ccc4O3)(CC1)[C@@H](O)C2)c1cc(C(F)F)no1] |
| 217 | 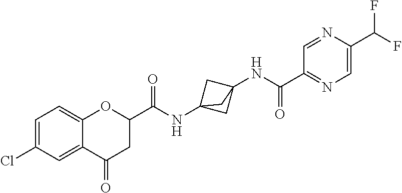 [SMILES: O=C(NC12CC(NC(=O)C3CC(=O)c4cc(Cl)ccc4O3)(C1)C2)c1cnc(C(F)F)cn1] |
| 218 | 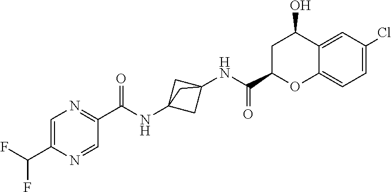 [SMILES: O=C(NC12CC(NC(=O)[C@H]3C[C@@H](O)c4cc(Cl)ccc4O3)(C1)C2)c1cnc(C(F)F)cn1] |
| 219 | 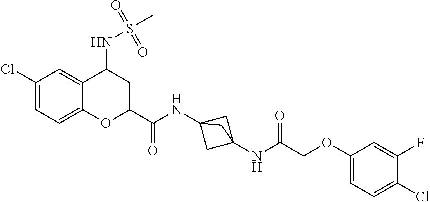 [SMILES: CS(=O)(=O)NC1CC(C(=O)NC23CC(NC(=O)COc4ccc(Cl)c(F)c4)(C2)C3)Oc2ccc(Cl)cc21] |
| 220 | 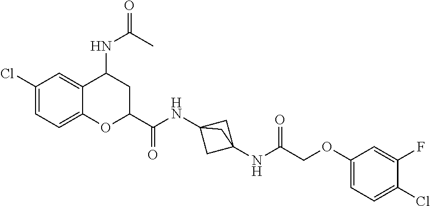 [SMILES: CC(=O)NC1CC(C(=O)NC23CC(NC(=O)COc4ccc(Cl)c(F)c4)(C2)C3)Oc2ccc(Cl)cc21] |
| 221 | 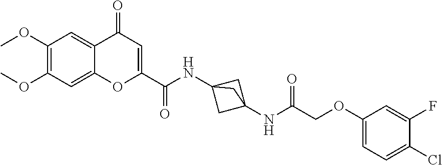 [SMILES: COc1cc2oc(C(=O)NC34CC(NC(=O)COc5ccc(Cl)c(F)c5)(C3)C4)cc(=O)c2cc1OC] |
| 222 | 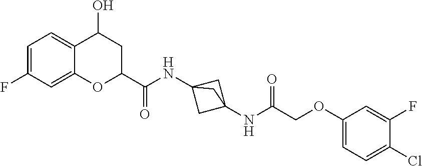 [SMILES: O=C(COc1ccc(Cl)c(F)c1)NC12CC(NC(=O)C3CC(O)c4ccc(F)cc4O3)(C1)C2] |
| 223 | 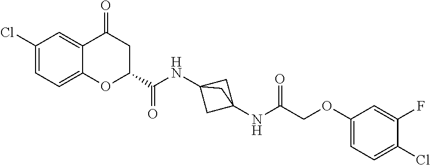 [SMILES: O=C(COc1ccc(Cl)c(F)c1)NC12CC(NC(=O)[C@H]3CC(=O)c4cc(Cl)ccc4O3)(C1)C2] |
| 224 | 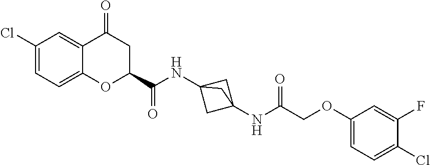 [SMILES: O=C(COc1ccc(Cl)c(F)c1)NC12CC(NC(=O)[C@@H]3CC(=O)c4cc(Cl)ccc4O3)(C1)C2] |
| 225 | 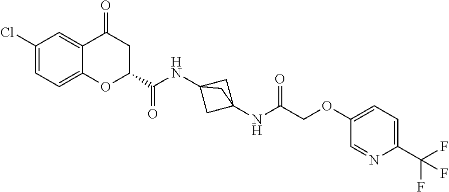 [SMILES: O=C(COc1ccc(C(F)(F)F)nc1)NC12CC(NC(=O)[C@H]3CC(=O)c4cc(Cl)ccc4O3)(C1)C2] |
| 226 | 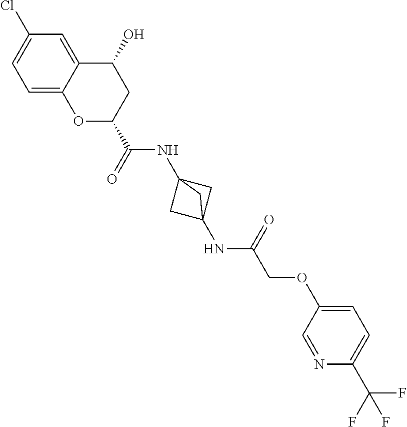 [SMILES: O=C(COc1ccc(C(F)(F)F)nc1)NC12CC(NC(=O)[C@H]3C[C@@H](O)c4cc(Cl)ccc4O3)(C1)C2] |
| 227 | 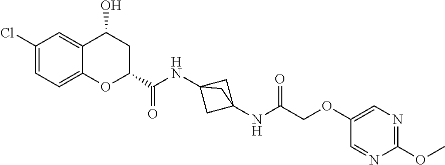 [SMILES: COc1ncc(OCC(=O)NC23CC(NC(=O)[C@H]4C[C@@H](O)c5cc(Cl)ccc5O4)(C2)C3)cn1] |
| 228 | 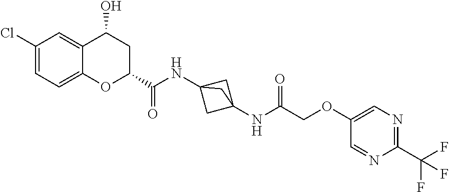 [SMILES: O=C(COc1cnc(C(F)(F)F)nc1)NC12CC(NC(=O)[C@H]3C[C@@H](O)c4cc(Cl)ccc4O3)(C1)C2] |
| 229 | 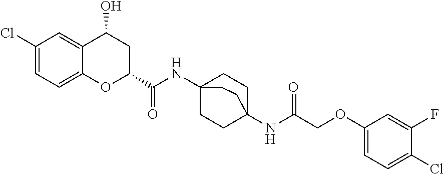 [SMILES: O=C(COc1ccc(Cl)c(F)c1)NC12CCC(NC(=O)[C@H]3C[C@@H](O)c4cc(Cl)ccc4O3)(CC1)CC2] |
| 230 | 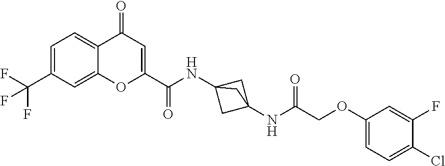 [SMILES: O=C(COc1ccc(Cl)c(F)c1)NC12CC(NC(=O)c3cc(=O)c4ccc(C(F)(F)F)cc4o3)(C1)C2] |
| 231 | 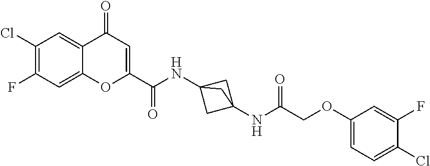 [SMILES: O=C(COc1ccc(Cl)c(F)c1)NC12CC(NC(=O)c3cc(=O)c4cc(Cl)c(F)cc4o3)(C1)C2] |
| 232 | 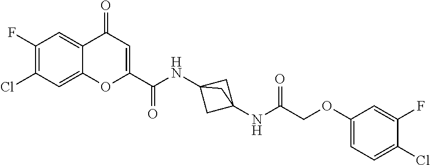 [SMILES: O=C(COc1ccc(Cl)c(F)c1)NC12CC(NC(=O)c3cc(=O)c4cc(F)c(Cl)cc4o3)(C1)C2] |
| 233 | 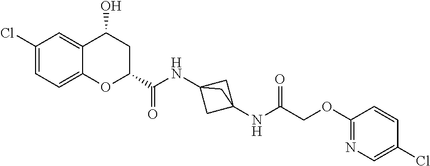 [SMILES: O=C(COc1ccc(Cl)cn1)NC12CC(NC(=O)[C@H]3C[C@@H](O)c4cc(Cl)ccc4O3)(C1)C2] |
| 234 | 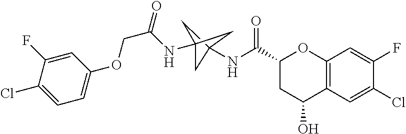 [SMILES: O=C(COc1ccc(Cl)c(F)c1)NC12CC(NC(=O)[C@H]3C[C@@H](O)c4cc(Cl)c(F)cc4O3)(C1)C2] |
| 235 | 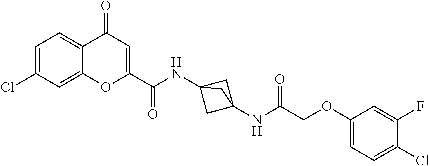 [SMILES: O=C(COc1ccc(Cl)c(F)c1)NC12CC(NC(=O)c3cc(=O)c4ccc(Cl)cc4o3)(C1)C2] |
| 236 | 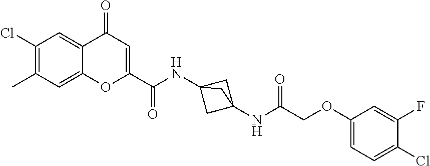 [SMILES: Cc1cc2oc(C(=O)NC34CC(NC(=O)COc5ccc(Cl)c(F)c5)(C3)C4)cc(=O)c2cc1Cl] |
| 237 | 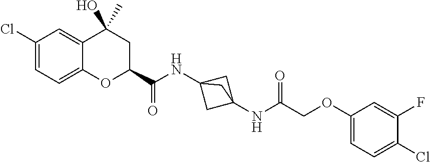 [SMILES: C[C@]1(O)C[C@@H](C(=O)NC23CC(NC(=O)COc4ccc(Cl)c(F)c4)(C2)C3)Oc2ccc(Cl)cc21] |
| 238 | 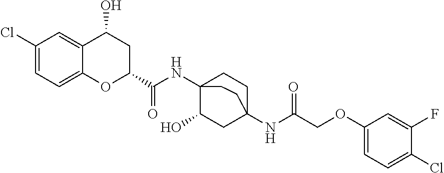 [SMILES: O=C(COc1ccc(Cl)c(F)c1)NC12CCC(NC(=O)[C@H]3C[C@@H](O)c4cc(Cl)ccc4O3)(CC1)[C@@H](O)C2] |
| 239 | 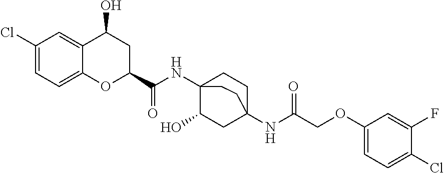 [SMILES: O=C(COc1ccc(Cl)c(F)c1)NC12CCC(NC(=O)[C@@H]3C[C@H](O)c4cc(Cl)ccc4O3)(CC1)[C@@H](O)C2] |
| 240 | 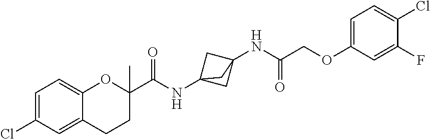 [SMILES: CC1(C(=O)NC23CC(NC(=O)COc4ccc(Cl)c(F)c4)(C2)C3)CCc2cc(Cl)ccc2O1] |
| 241 | 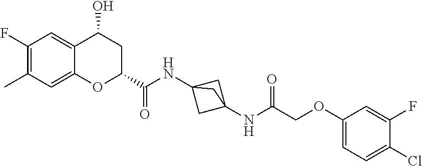 [SMILES: Cc1cc2c(cc1F)[C@H](O)C[C@H](C(=O)NC13CC(NC(=O)COc4ccc(Cl)c(F)c4)(C1)C3)O2] |
| 242 | 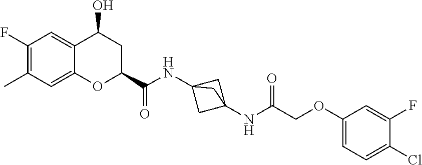 [SMILES: Cc1cc2c(cc1F)[C@@H](O)C[C@@H](C(=O)NC13CC(NC(=O)COc4ccc(Cl)c(F)c4)(C1)C3)O2] |
| 243 | 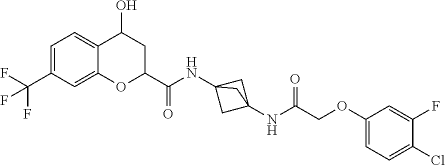 [SMILES: O=C(COc1ccc(Cl)c(F)c1)NC12CC(NC(=O)C3CC(O)c4ccc(C(F)(F)F)cc4O3)(C1)C2] |
| 244 | 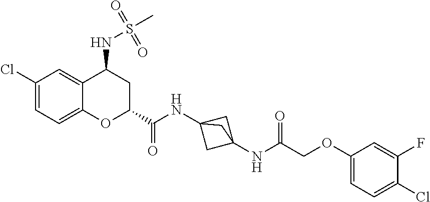 [SMILES: CS(=O)(=O)N[C@H]1C[C@H](C(=O)NC23CC(NC(=O)COc4ccc(Cl)c(F)c4)(C2)C3)Oc2ccc(Cl)cc21] |
| 245 | 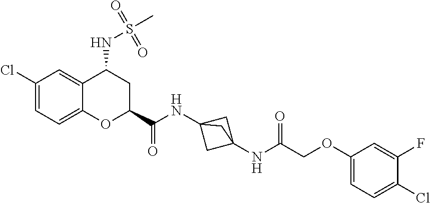 [SMILES: CS(=O)(=O)N[C@@H]1C[C@@H](C(=O)NC23CC(NC(=O)COc4ccc(Cl)c(F)c4)(C2)C3)Oc2ccc(Cl)cc21] |
| 246 | 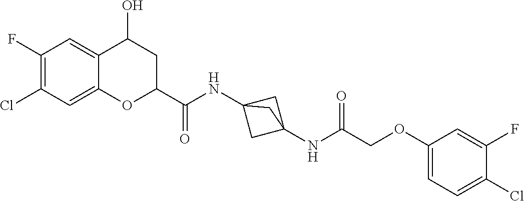 [SMILES: O=C(COc1ccc(Cl)c(F)c1)NC12CC(NC(=O)C3CC(O)c4cc(F)c(Cl)cc4O3)(C1)C2] |
| 247 | 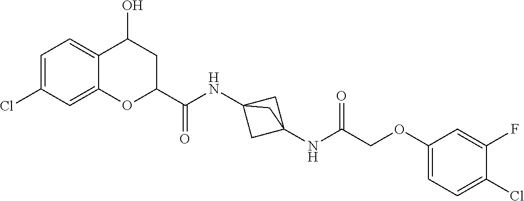 [SMILES: O=C(COc1ccc(Cl)c(F)c1)NC12CC(NC(=O)C3CC(O)c4ccc(Cl)cc4O3)(C1)C2] |
| 248 | 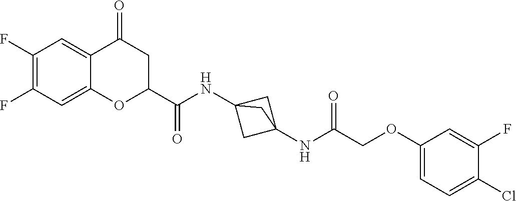 [SMILES: O=C(COc1ccc(Cl)c(F)c1)NC12CC(NC(=O)C3CC(=O)c4cc(F)c(F)cc4O3)(C1)C2] |
| 249 | 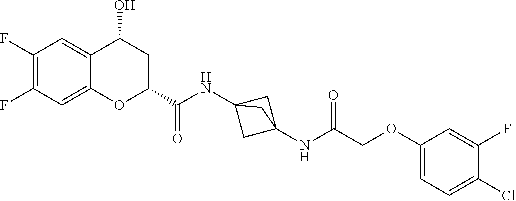 [SMILES: O=C(COc1ccc(Cl)c(F)c1)NC12CC(NC(=O)[C@H]3C[C@@H](O)c4cc(F)c(F)cc4O3)(C1)C2] |
| 250 | 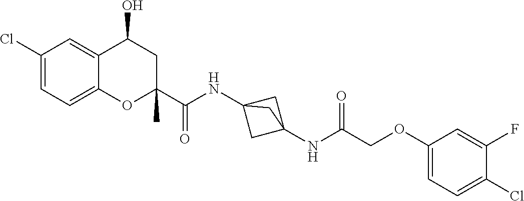 [SMILES: C[C@]1(C(=O)NC23CC(NC(=O)COc4ccc(Cl)c(F)c4)(C2)C3)C[C@H](O)c2cc(Cl)ccc2O1] |
| 251 | 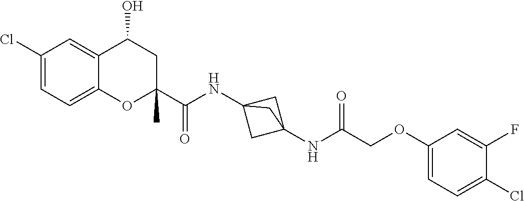 [SMILES: C[C@]1(C(=O)NC23CC(NC(=O)COc4ccc(Cl)c(F)c4)(C2)C3)C[C@@H](O)c2cc(Cl)ccc2O1] |
| 252 | 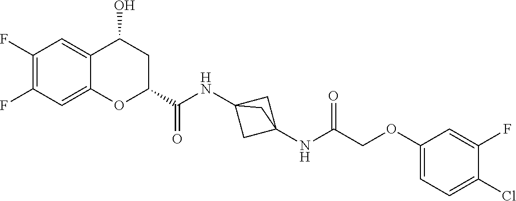 [SMILES: O=C(COc1ccc(Cl)c(F)c1)NC12CC(NC(=O)[C@H]3C[C@@H](O)c4cc(F)c(F)cc4O3)(C1)C2] |
| 253 | 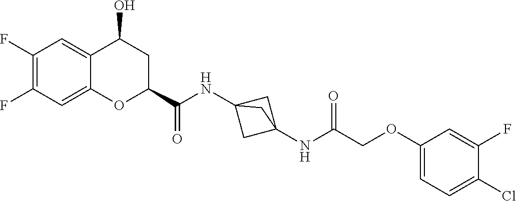 [SMILES: O=C(COc1ccc(Cl)c(F)c1)NC12CC(NC(=O)[C@@H]3C[C@H](O)c4cc(F)c(F)cc4O3)(C1)C2] |
| 254 | 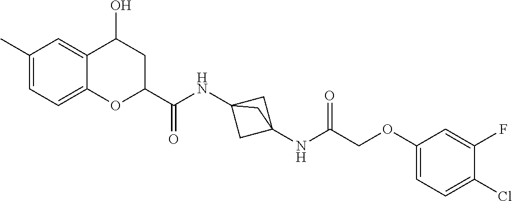 [SMILES: Cc1ccc2c(c1)C(O)CC(C(=O)NC13CC(NC(=O)COc4ccc(Cl)c(F)c4)(C1)C3)O2] |
| 255 | 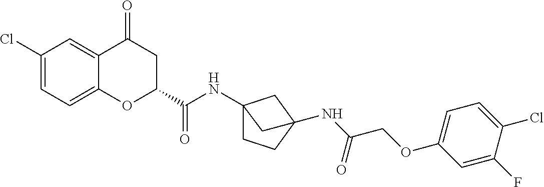 [SMILES: O=C(COc1ccc(Cl)c(F)c1)NC12CCC(NC(=O)[C@H]3CC(=O)c4cc(Cl)ccc4O3)(C1)C2] |
| 256 | 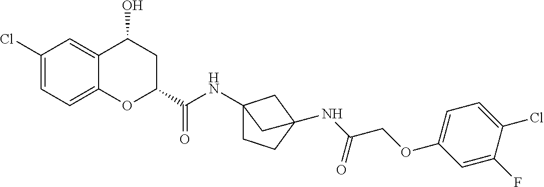 [SMILES: O=C(COc1ccc(Cl)c(F)c1)NC12CCC(NC(=O)[C@H]3C[C@@H](O)c4cc(Cl)ccc4O3)(C1)C2] |
| 257 | 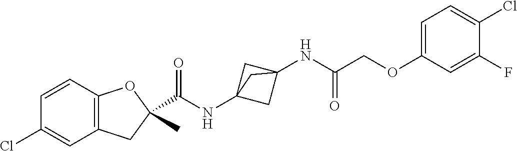 [SMILES: C[C@@]1(C(=O)NC23CC(NC(=O)COc4ccc(Cl)c(F)c4)(C2)C3)Cc2cc(Cl)ccc2O1] |
| 258 | 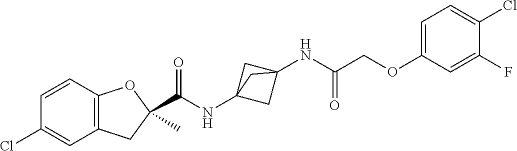 [SMILES: C[C@]1(C(=O)NC23CC(NC(=O)COc4ccc(Cl)c(F)c4)(C2)C3)Cc2cc(Cl)ccc2O1] |
| 259 | 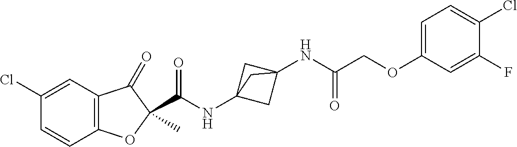 [SMILES: C[C@]1(C(=O)NC23CC(NC(=O)COc4ccc(Cl)c(F)c4)(C2)C3)Oc2ccc(Cl)cc2C1=O] |
| 260 | 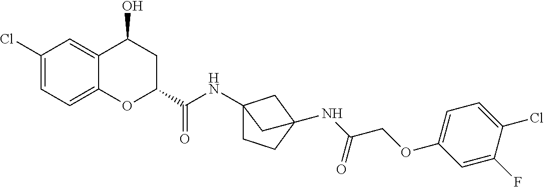 [SMILES: O=C(COc1ccc(Cl)c(F)c1)NC12CCC(NC(=O)[C@H]3C[C@H](O)c4cc(Cl)ccc4O3)(C1)C2] |
| 261 | 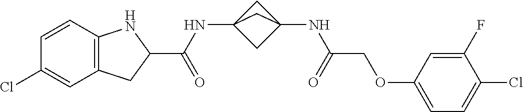 [SMILES: O=C(COc1ccc(Cl)c(F)c1)NC12CC(NC(=O)C3Cc4cc(Cl)ccc4N3)(C1)C2] |
| 262 | 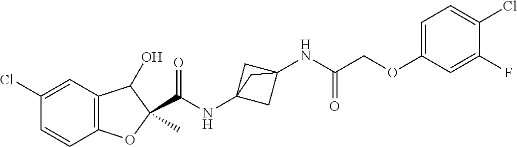 [SMILES: C[C@]1(C(=O)NC23CC(NC(=O)COc4ccc(Cl)c(F)c4)(C2)C3)Oc2ccc(Cl)cc2C1O] |
| 263 | 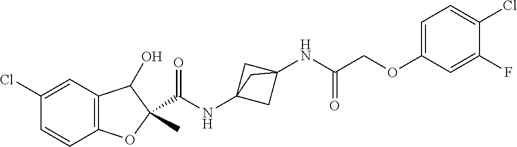 [SMILES: C[C@@]1(C(=O)NC23CC(NC(=O)COc4ccc(Cl)c(F)c4)(C2)C3)Oc2ccc(Cl)cc2C1O] |
| 264 | 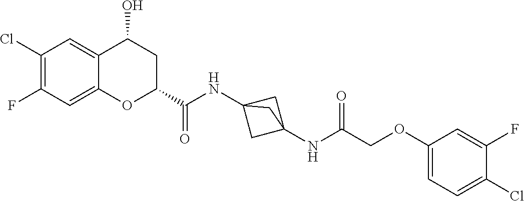 [SMILES: O=C(COc1ccc(Cl)c(F)c1)NC12CC(NC(=O)[C@H]3C[C@@H](O)c4cc(Cl)c(F)cc4O3)(C1)C2] |
| 265 | 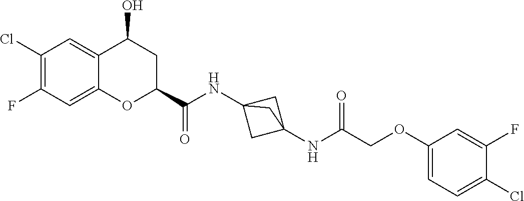 [SMILES: O=C(COc1ccc(Cl)c(F)c1)NC12CC(NC(=O)[C@@H]3C[C@H](O)c4cc(Cl)c(F)cc4O3)(C1)C2] |
| 266 | 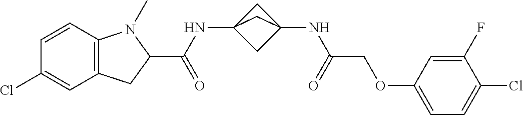 [SMILES: CN1c2ccc(Cl)cc2CC1C(=O)NC12CC(NC(=O)COc3ccc(Cl)c(F)c3)(C1)C2] |
| 267 | 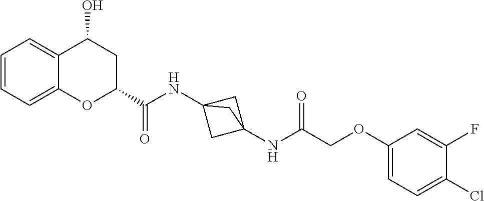 [SMILES: O=C(COc1ccc(Cl)c(F)c1)NC12CC(NC(=O)[C@H]3C[C@@H](O)c4ccccc4O3)(C1)C2] |
| 268 | 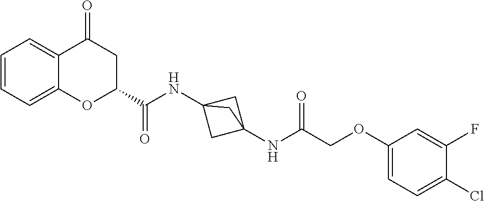 [SMILES: O=C(COc1ccc(Cl)c(F)c1)NC12CC(NC(=O)[C@H]3CC(=O)c4ccccc4O3)(C1)C2] |
| 269 | 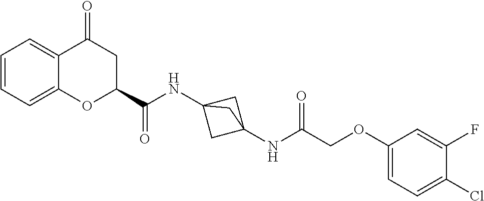 [SMILES: O=C(COc1ccc(Cl)c(F)c1)NC12CC(NC(=O)[C@@H]3CC(=O)c4ccccc4O3)(C1)C2] |
| 270 | 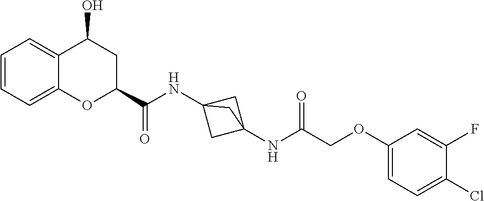 [SMILES: O=C(COc1ccc(Cl)c(F)c1)NC12CC(NC(=O)[C@@H]3C[C@H](O)c4ccccc4O3)(C1)C2] |
| 271 | 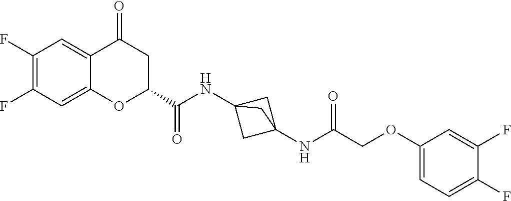 [SMILES: O=C(COc1ccc(F)c(F)c1)NC12CC(NC(=O)[C@H]3CC(=O)c4cc(F)c(F)cc4O3)(C1)C2] |
| 272 | 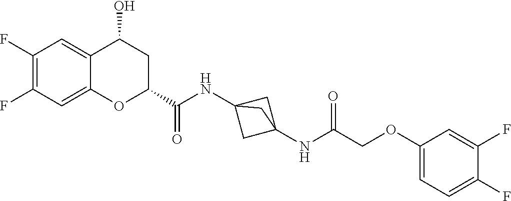 [SMILES: O=C(COc1ccc(F)c(F)c1)NC12CC(NC(=O)[C@H]3C[C@@H](O)c4cc(F)c(F)cc4O3)(C1)C2] |
| 273 | 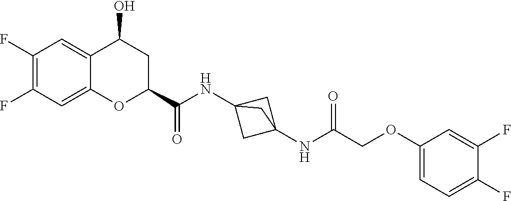 [SMILES: O=C(COc1ccc(F)c(F)c1)NC12CC(NC(=O)[C@@H]3C[C@H](O)c4cc(F)c(F)cc4O3)(C1)C2] |
| 274 | 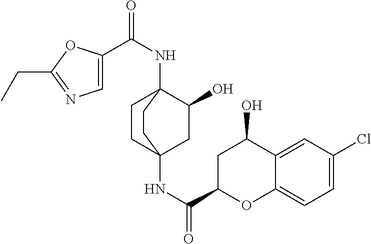 [SMILES: CCc1ncc(C(=O)NC23CCC(NC(=O)[C@H]4C[C@@H](O)c5cc(Cl)ccc5O4)(CC2)C[C@@H]3O)o1] |
| 275 | 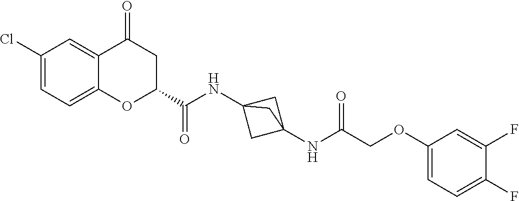 [SMILES: O=C(COc1ccc(F)c(F)c1)NC12CC(NC(=O)[C@H]3CC(=O)c4cc(Cl)ccc4O3)(C1)C2] |
| 276 | 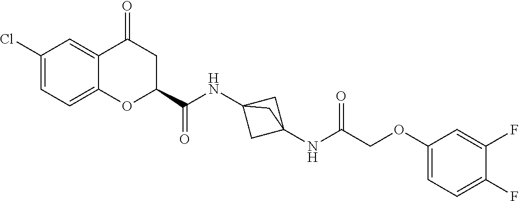 [SMILES: O=C(COc1ccc(F)c(F)c1)NC12CC(NC(=O)[C@@H]3CC(=O)c4cc(Cl)ccc4O3)(C1)C2] |
| 277 | 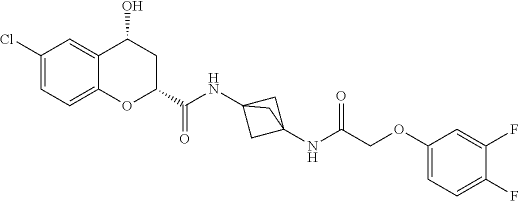 [SMILES: O=C(COc1ccc(F)c(F)c1)NC12CC(NC(=O)[C@H]3C[C@@H](O)c4cc(Cl)ccc4O3)(C1)C2] |
| 278 | 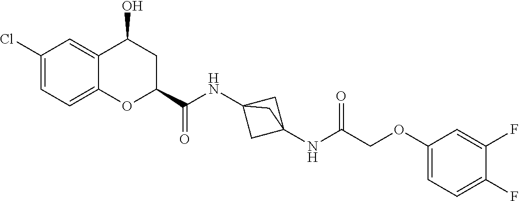 [SMILES: O=C(COc1ccc(F)c(F)c1)NC12CC(NC(=O)[C@@H]3C[C@H](O)c4cc(Cl)ccc4O3)(C1)C2] |
| 279 | 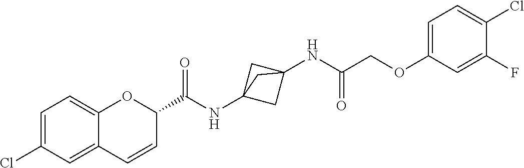 [SMILES: O=C(COc1ccc(Cl)c(F)c1)NC12CC(NC(=O)[C@@H]3C=Cc4cc(Cl)ccc4O3)(C1)C2] |
| 280 | 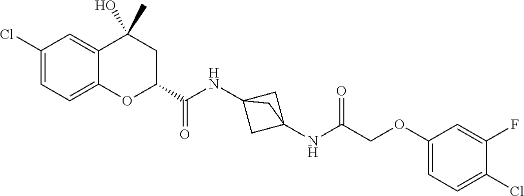 [SMILES: C[C@@]1(O)C[C@H](C(=O)NC23CC(NC(=O)COc4ccc(Cl)c(F)c4)(C2)C3)Oc2ccc(Cl)cc21] |
| 281 | 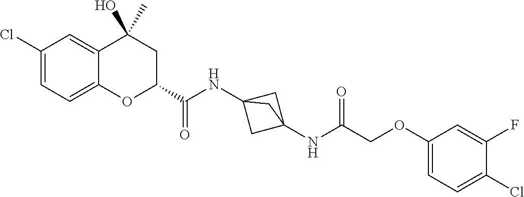 [SMILES: C[C@]1(O)C[C@H](C(=O)NC23CC(NC(=O)COc4ccc(Cl)c(F)c4)(C2)C3)Oc2ccc(Cl)cc21] |
| 282 | 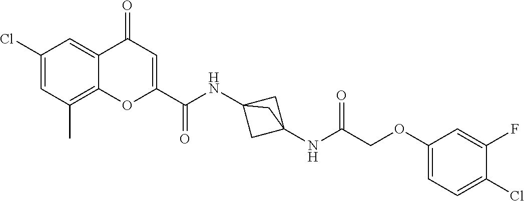 [SMILES: Cc1cc(Cl)cc2c(=O)cc(C(=O)NC34CC(NC(=O)COc5ccc(Cl)c(F)c5)(C3)C4)oc12] |
| 283 | 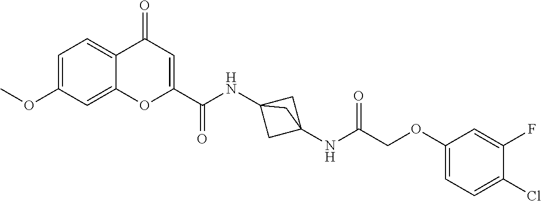 [SMILES: COc1ccc2c(=O)cc(C(=O)NC34CC(NC(=O)COc5ccc(Cl)c(F)c5)(C3)C4)oc2c1] |
| 284 | 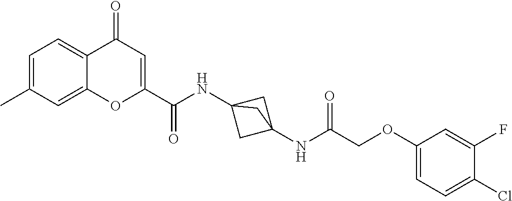 [SMILES: Cc1ccc2c(=O)cc(C(=O)NC34CC(NC(=O)COc5ccc(Cl)c(F)c5)(C3)C4)oc2c1] |
| 285 | 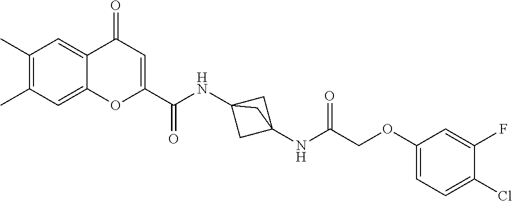 [SMILES: Cc1cc2oc(C(=O)NC34CC(NC(=O)COc5ccc(Cl)c(F)c5)(C3)C4)cc(=O)c2cc1C] |
| 286 | 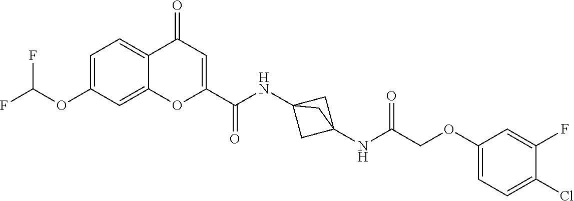 [SMILES: O=C(COc1ccc(Cl)c(F)c1)NC12CC(NC(=O)c3cc(=O)c4ccc(OC(F)F)cc4o3)(C1)C2] |
| 287 | 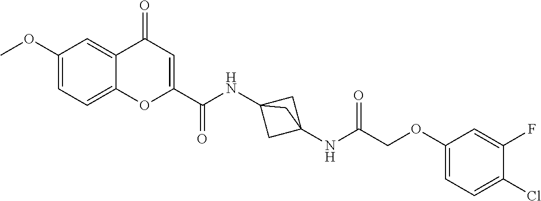 [SMILES: COc1ccc2oc(C(=O)NC34CC(NC(=O)COc5ccc(Cl)c(F)c5)(C3)C4)cc(=O)c2c1] |
| 288 | 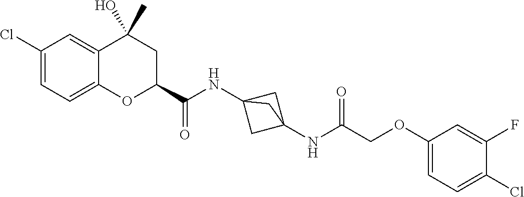 [SMILES: C[C@@]1(O)C[C@@H](C(=O)NC23CC(NC(=O)COc4ccc(Cl)c(F)c4)(C2)C3)Oc2ccc(Cl)cc21] |
| 289 | 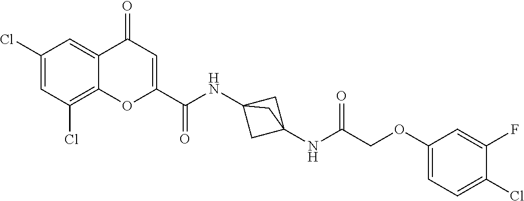 [SMILES: O=C(COc1ccc(Cl)c(F)c1)NC12CC(NC(=O)c3cc(=O)c4cc(Cl)cc(Cl)c4o3)(C1)C2] |
| 290 | 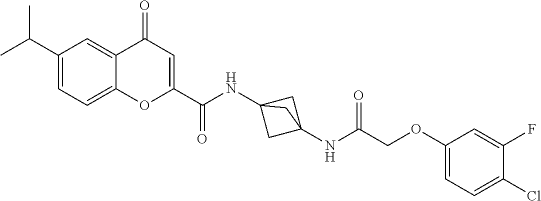 [SMILES: CC(C)c1ccc2oc(C(=O)NC34CC(NC(=O)COc5ccc(Cl)c(F)c5)(C3)C4)cc(=O)c2c1] |
| 291 | 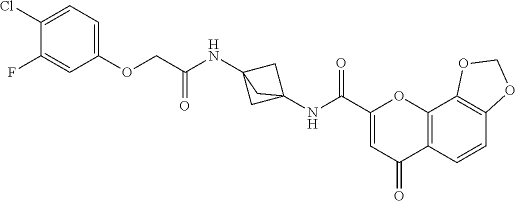 [SMILES: O=C(COc1ccc(Cl)c(F)c1)NC12CC(NC(=O)c3cc(=O)c4ccc5c(c4o3)OCO5)(C1)C2] |
| 292 | 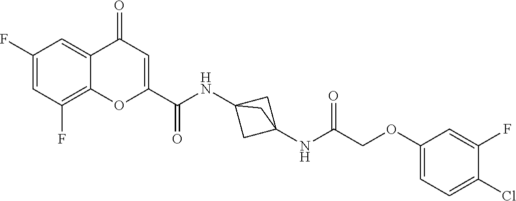 [SMILES: O=C(COc1ccc(Cl)c(F)c1)NC12CC(NC(=O)c3cc(=O)c4cc(F)cc(F)c4o3)(C1)C2] |
| 293 | 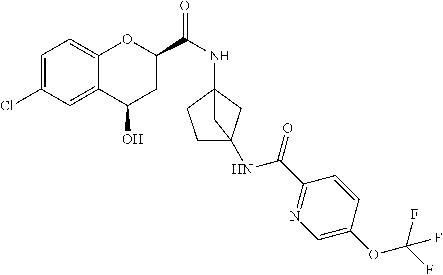 [SMILES: O=C(NC12CCC(NC(=O)[C@H]3C[C@@H](O)c4cc(Cl)ccc4O3)(C1)C2)c1ccc(OC(F)(F)F)cn1] |
| 294 | 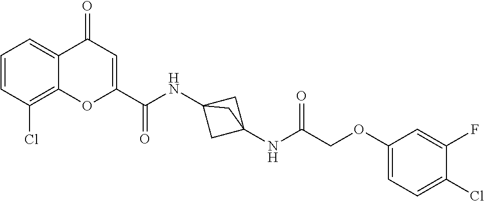 [SMILES: O=C(COc1ccc(Cl)c(F)c1)NC12CC(NC(=O)c3cc(=O)c4cccc(Cl)c4o3)(C1)C2] |
| 295 | 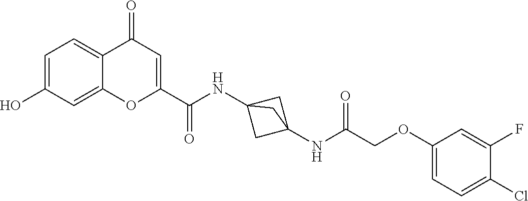 [SMILES: O=C(COc1ccc(Cl)c(F)c1)NC12CC(NC(=O)c3cc(=O)c4ccc(O)cc4o3)(C1)C2] |
| 296 | 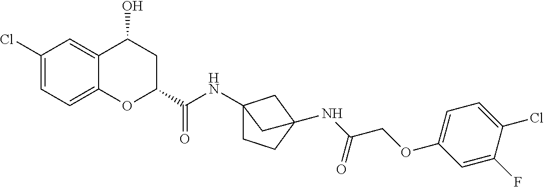 [SMILES: O=C(COc1ccc(Cl)c(F)c1)NC12CCC(NC(=O)[C@H]3C[C@@H](O)c4cc(Cl)ccc4O3)(C1)C2] |
| 297 | 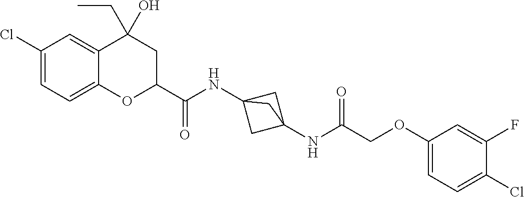 [SMILES: CCC1(O)CC(C(=O)NC23CC(NC(=O)COc4ccc(Cl)c(F)c4)(C2)C3)Oc2ccc(Cl)cc21] |
| 298 | 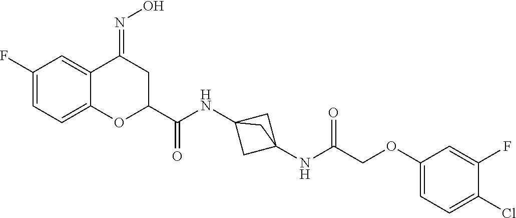 [SMILES: O=C(COc1ccc(Cl)c(F)c1)NC12CC(NC(=O)C3C/C(=N\O)c4cc(F)ccc4O3)(C1)C2] |
| 299 | 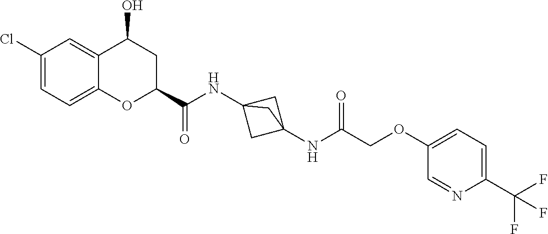 [SMILES: O=C(COc1ccc(C(F)(F)F)nc1)NC12CC(NC(=O)[C@@H]3C[C@H](O)c4cc(Cl)ccc4O3)(C1)C2] |
| 300 | 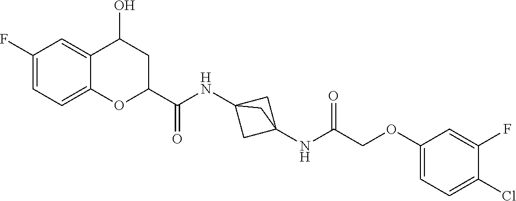 [SMILES: O=C(COc1ccc(Cl)c(F)c1)NC12CC(NC(=O)C3CC(O)c4cc(F)ccc4O3)(C1)C2] |
| 301 | 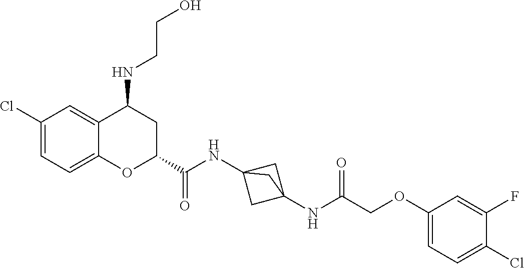 [SMILES: O=C(COc1ccc(Cl)c(F)c1)NC12CC(NC(=O)[C@H]3C[C@H](NCCO)c4cc(Cl)ccc4O3)(C1)C2] |
| 302 | 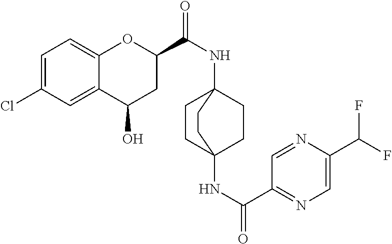 [SMILES: O=C(NC12CCC(NC(=O)[C@H]3C[C@@H](O)c4cc(Cl)ccc4O3)(CC1)CC2)c1cnc(C(F)F)cn1] |
| 303 | 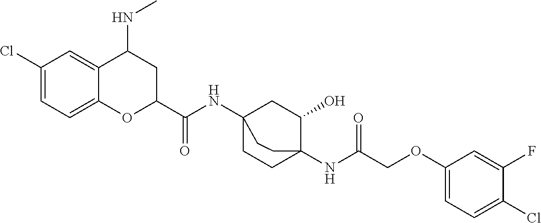 [SMILES: CNC1CC(C(=O)NC23CCC(NC(=O)COc4ccc(Cl)c(F)c4)(CC2)[C@@H](O)C3)Oc2ccc(Cl)cc21] |
| 304 | 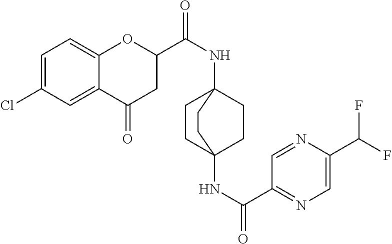 [SMILES: O=C(NC12CCC(NC(=O)C3CC(=O)c4cc(Cl)ccc4O3)(CC1)CC2)c1cnc(C(F)F)cn1] |
| 305 | 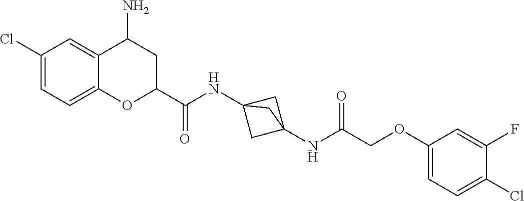 [SMILES: NC1CC(C(=O)NC23CC(NC(=O)COc4ccc(Cl)c(F)c4)(C2)C3)Oc2ccc(Cl)cc21] |
| 306 | 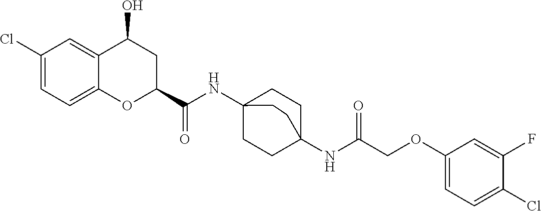 [SMILES: O=C(COc1ccc(Cl)c(F)c1)NC12CCC(NC(=O)[C@@H]3C[C@H](O)c4cc(Cl)ccc4O3)(CC1)CC2] |
| 307 | 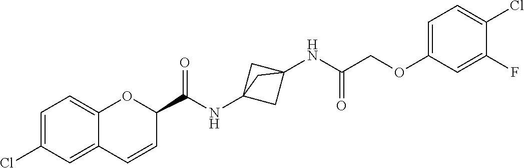 [SMILES: O=C(COc1ccc(Cl)c(F)c1)NC12CC(NC(=O)[C@H]3C=Cc4cc(Cl)ccc4O3)(C1)C2] |
| 308 | 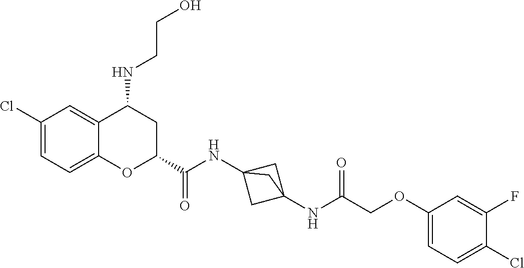 [SMILES: O=C(COc1ccc(Cl)c(F)c1)NC12CC(NC(=O)[C@H]3C[C@@H](NCCO)c4cc(Cl)ccc4O3)(C1)C2] |
| 309 | 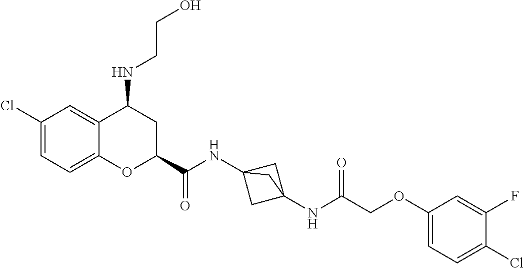 [SMILES: O=C(COc1ccc(Cl)c(F)c1)NC12CC(NC(=O)[C@@H]3C[C@H](NCCO)c4cc(Cl)ccc4O3)(C1)C2] |
| 310 | 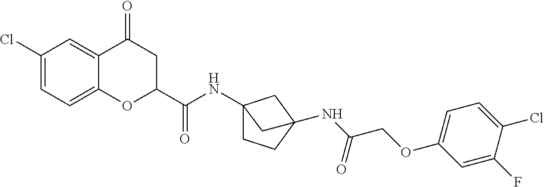 [SMILES: O=C(COc1ccc(Cl)c(F)c1)NC12CCC(NC(=O)C3CC(=O)c4cc(Cl)ccc4O3)(C1)C2] |
| 311 | 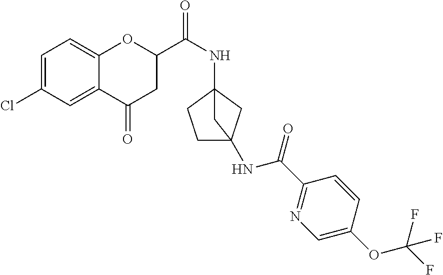 [SMILES: O=C(NC12CCC(NC(=O)C3CC(=O)c4cc(Cl)ccc4O3)(C1)C2)c1ccc(OC(F)(F)F)cn1] |
| 312 | 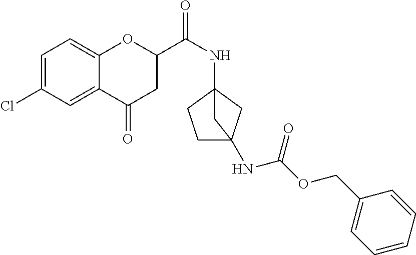 [SMILES: O=C(NC12CCC(NC(=O)C3CC(=O)c4cc(Cl)ccc4O3)(C1)C2)OCc1ccccc1] |
| 313 | 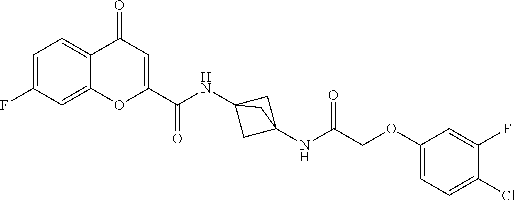 [SMILES: O=C(COc1ccc(Cl)c(F)c1)NC12CC(NC(=O)c3cc(=O)c4ccc(F)cc4o3)(C1)C2] |
| 314 | 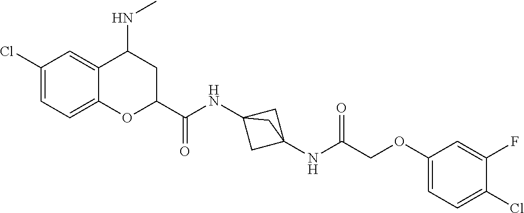 [SMILES: CNC1CC(C(=O)NC23CC(NC(=O)COc4ccc(Cl)c(F)c4)(C2)C3)Oc2ccc(Cl)cc21] |
| 315 | 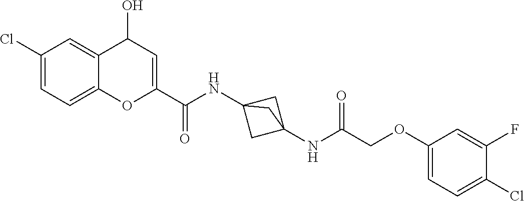 [SMILES: O=C(COc1ccc(Cl)c(F)c1)NC12CC(NC(=O)C3=CC(O)c4cc(Cl)ccc4O3)(C1)C2] |
| 316 | 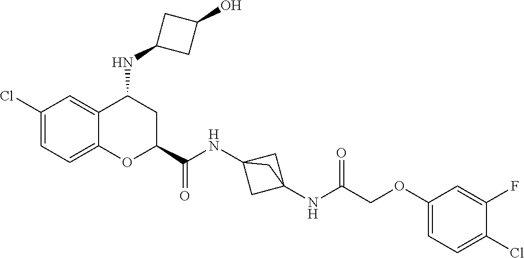 [SMILES: O=C(COc1ccc(Cl)c(F)c1)NC12CC(NC(=O)[C@@H]3C[C@@H](N[C@H]4C[C@@H](O)C4)c4cc(Cl)ccc4O3)(C1)C2] |
| 317 | 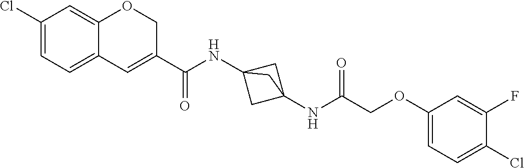 [SMILES: O=C(COc1ccc(Cl)c(F)c1)NC12CC(NC(=O)C3=Cc4ccc(Cl)cc4OC3)(C1)C2] |
| 318 | 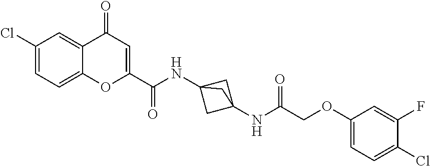 [SMILES: O=C(COc1ccc(Cl)c(F)c1)NC12CC(NC(=O)c3cc(=O)c4cc(Cl)ccc4o3)(C1)C2] |
| 319 | 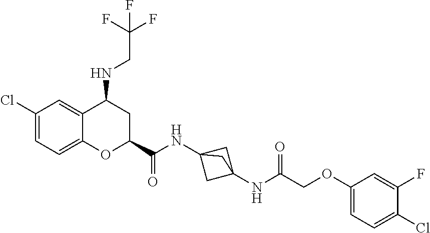 [SMILES: O=C(COc1ccc(Cl)c(F)c1)NC12CC(NC(=O)[C@@H]3C[C@H](NCC(F)(F)F)c4cc(Cl)ccc4O3)(C1)C2] |
| 320 | 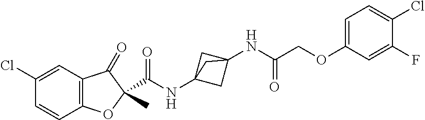 [SMILES: C[C@@]1(C(=O)NC23CC(NC(=O)COc4ccc(Cl)c(F)c4)(C2)C3)Oc2ccc(Cl)cc2C1=O] |
| 321 | 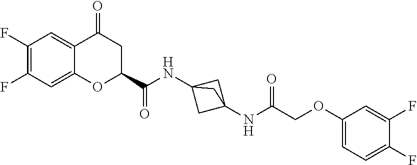 [SMILES: O=C(COc1ccc(F)c(F)c1)NC12CC(NC(=O)[C@@H]3CC(=O)c4cc(F)c(F)cc4O3)(C1)C2] |
| 322 | 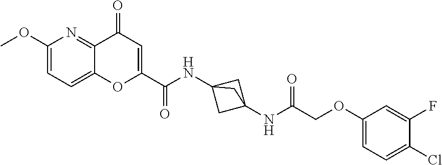 [SMILES: COc1ccc2oc(C(=O)NC34CC(NC(=O)COc5ccc(Cl)c(F)c5)(C3)C4)cc(=O)c2n1] |
| 323 | 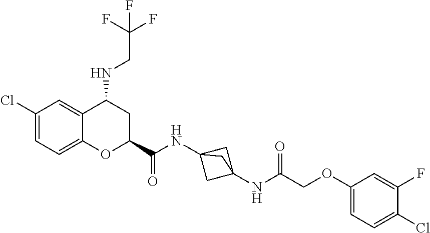 [SMILES: O=C(COc1ccc(Cl)c(F)c1)NC12CC(NC(=O)[C@@H]3C[C@@H](NCC(F)(F)F)c4cc(Cl)ccc4O3)(C1)C2] |
| 324 | 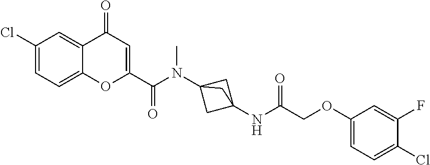 [SMILES: CN(C(=O)c1cc(=O)c2cc(Cl)ccc2o1)C12CC(NC(=O)COc3ccc(Cl)c(F)c3)(C1)C2] |
| 325 | 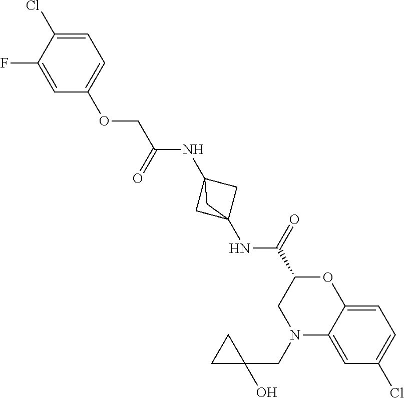 [SMILES: O=C(COc1ccc(Cl)c(F)c1)NC12CC(NC(=O)[C@H]3CN(CC4(O)CC4)c4cc(Cl)ccc4O3)(C1)C2] |
| 326 | 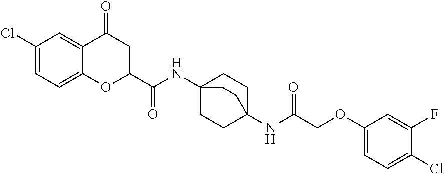 [SMILES: O=C(COc1ccc(Cl)c(F)c1)NC12CCC(NC(=O)C3CC(=O)c4cc(Cl)ccc4O3)(CC1)CC2] |
| 327 | 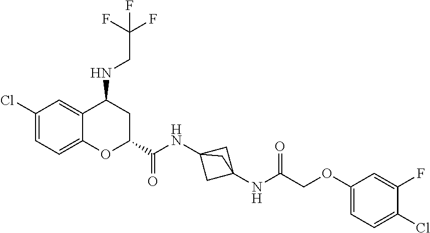 [SMILES: O=C(COc1ccc(Cl)c(F)c1)NC12CC(NC(=O)[C@H]3C[C@H](NCC(F)(F)F)c4cc(Cl)ccc4O3)(C1)C2] |
| 328 | 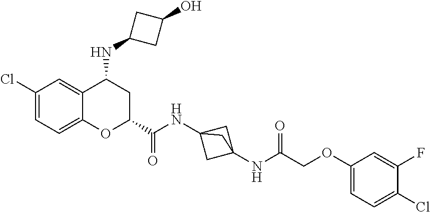 [SMILES: O=C(COc1ccc(Cl)c(F)c1)NC12CC(NC(=O)[C@H]3C[C@@H](N[C@H]4C[C@@H](O)C4)c4cc(Cl)ccc4O3)(C1)C2] |
| 329 | 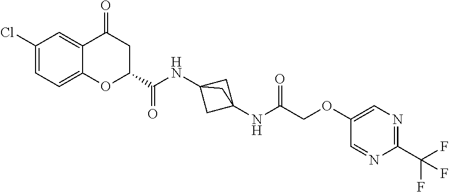 [SMILES: O=C(COc1cnc(C(F)(F)F)nc1)NC12CC(NC(=O)[C@H]3CC(=O)c4cc(Cl)ccc4O3)(C1)C2] |
| 330 | 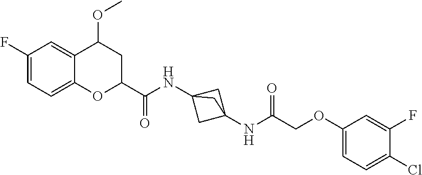 [SMILES: COC1CC(C(=O)NC23CC(NC(=O)COc4ccc(Cl)c(F)c4)(C2)C3)Oc2ccc(F)cc21] |
| 331 | 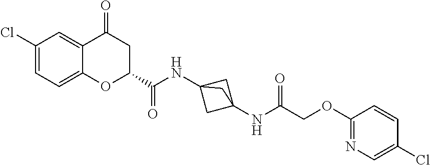 [SMILES: O=C(COc1ccc(Cl)cn1)NC12CC(NC(=O)[C@H]3CC(=O)c4cc(Cl)ccc4O3)(C1)C2] |
| 332 | 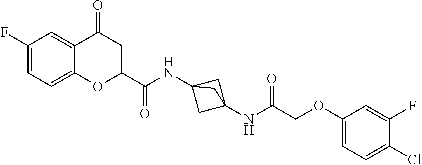 [SMILES: O=C(COc1ccc(Cl)c(F)c1)NC12CC(NC(=O)C3CC(=O)c4cc(F)ccc4O3)(C1)C2] |
| 333 | 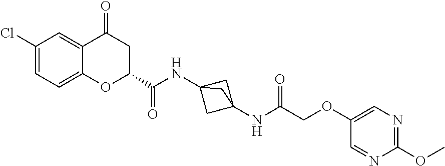 [SMILES: COc1ncc(OCC(=O)NC23CC(NC(=O)[C@H]4CC(=O)c5cc(Cl)ccc5O4)(C2)C3)cn1] |
| 334 | 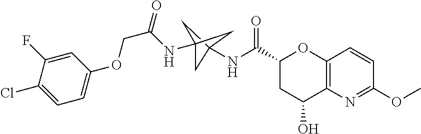 [SMILES: COc1ccc2c(n1)[C@H](O)C[C@H](C(=O)NC13CC(NC(=O)COc4ccc(Cl)c(F)c4)(C1)C3)O2] |
| 335 | 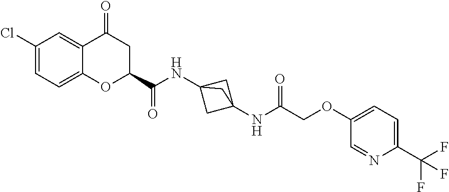 [SMILES: O=C(COc1ccc(C(F)(F)F)nc1)NC12CC(NC(=O)[C@@H]3CC(=O)c4cc(Cl)ccc4O3)(C1)C2] |
| 336 | 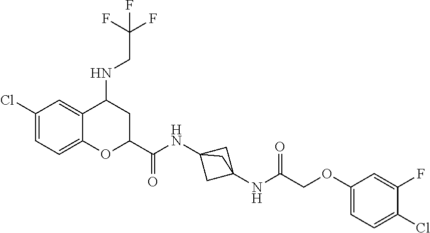 [SMILES: O=C(COc1ccc(Cl)c(F)c1)NC12CC(NC(=O)C3CC(NCC(F)(F)F)c4cc(Cl)ccc4O3)(C1)C2] |
| 337 | 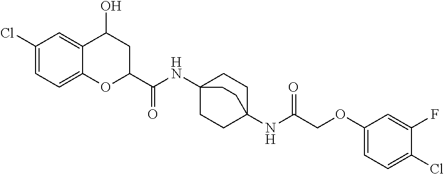 [SMILES: O=C(COc1ccc(Cl)c(F)c1)NC12CCC(NC(=O)C3CC(O)c4cc(Cl)ccc4O3)(CC1)CC2] |
| 338 | 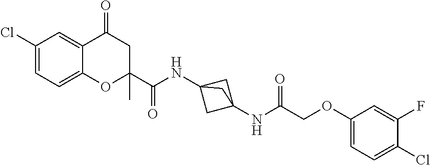 [SMILES: CC1(C(=O)NC23CC(NC(=O)COc4ccc(Cl)c(F)c4)(C2)C3)CC(=O)c2cc(Cl)ccc2O1] |
| 339 | 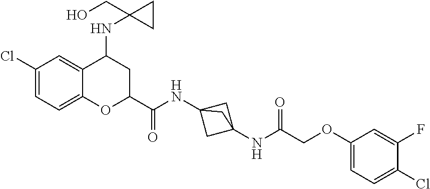 [SMILES: O=C(COc1ccc(Cl)c(F)c1)NC12CC(NC(=O)C3CC(NC4(CO)CC4)c4cc(Cl)ccc4O3)(C1)C2] |
| 340 | 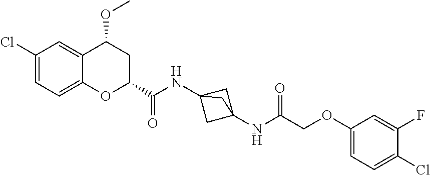 [SMILES: CO[C@@H]1C[C@H](C(=O)NC23CC(NC(=O)COc4ccc(Cl)c(F)c4)(C2)C3)Oc2ccc(Cl)cc21] |
| 341 | 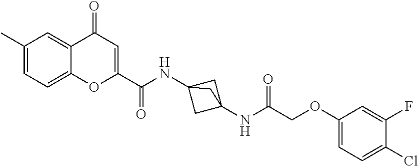 [SMILES: Cc1ccc2oc(C(=O)NC34CC(NC(=O)COc5ccc(Cl)c(F)c5)(C3)C4)cc(=O)c2c1] |
| 342 | 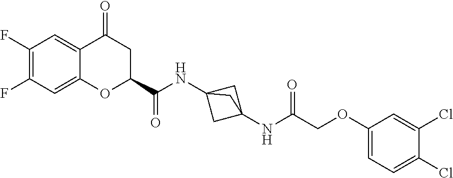 [SMILES: O=C(COc1ccc(Cl)c(Cl)c1)NC12CC(NC(=O)[C@@H]3CC(=O)c4cc(F)c(F)cc4O3)(C1)C2] |
| 343 | 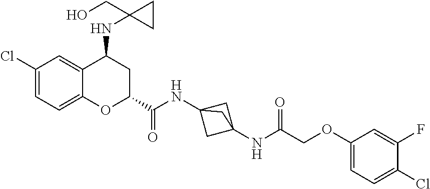 [SMILES: O=C(COc1ccc(Cl)c(F)c1)NC12CC(NC(=O)[C@H]3C[C@H](NC4(CO)CC4)c4cc(Cl)ccc4O3)(C1)C2] |
| 344 | 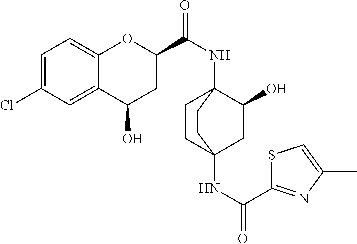 [SMILES: Cc1csc(C(=O)NC23CCC(NC(=O)[C@H]4C[C@@H](O)c5cc(Cl)ccc5O4)(CC2)[C@@H](O)C3)n1] |
| 345 | 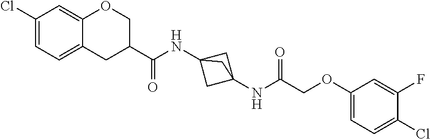 [SMILES: O=C(COc1ccc(Cl)c(F)c1)NC12CC(NC(=O)C3COc4cc(Cl)ccc4C3)(C1)C2] |
| 346 | 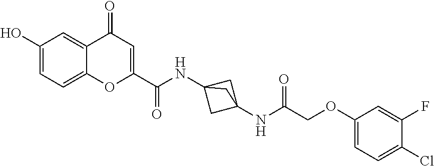 [SMILES: O=C(COc1ccc(Cl)c(F)c1)NC12CC(NC(=O)c3cc(=O)c4cc(O)ccc4o3)(C1)C2] |
| 347 | 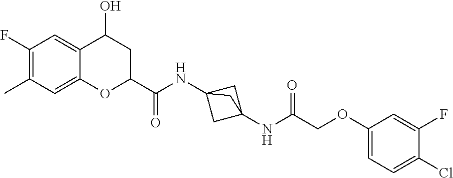 [SMILES: Cc1cc2c(cc1F)C(O)CC(C(=O)NC13CC(NC(=O)COc4ccc(Cl)c(F)c4)(C1)C3)O2] |
| 348 | 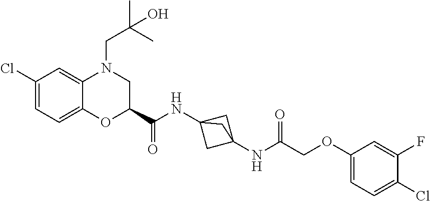 [SMILES: CC(C)(O)CN1C[C@@H](C(=O)NC23CC(NC(=O)COc4ccc(Cl)c(F)c4)(C2)C3)Oc2ccc(Cl)cc21] |
| 349 | 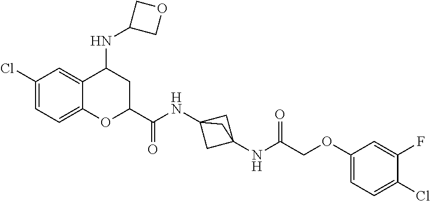 [SMILES: O=C(COc1ccc(Cl)c(F)c1)NC12CC(NC(=O)C3CC(NC4COC4)c4cc(Cl)ccc4O3)(C1)C2] |
| 350 | 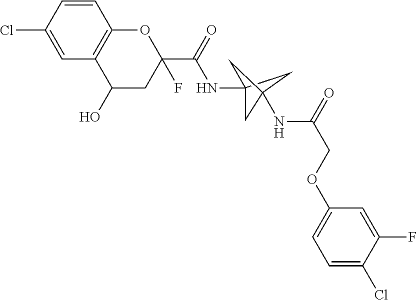 [SMILES: O=C(COc1ccc(Cl)c(F)c1)NC12CC(NC(=O)C3(F)CC(O)c4cc(Cl)ccc4O3)(C1)C2] |
| 351 | 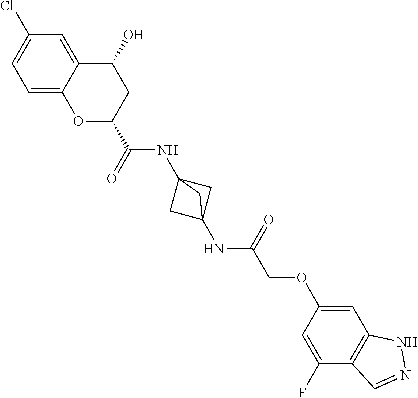 [SMILES: O=C(COc1cc(F)c2cn[nH]c2c1)NC12CC(NC(=O)[C@H]3C[C@@H](O)c4cc(Cl)ccc4O3)(C1)C2] |
| 352 | 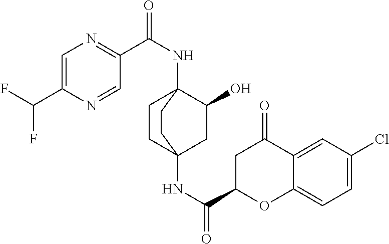 [SMILES: O=C(NC12CCC(NC(=O)[C@H]3CC(=O)c4cc(Cl)ccc4O3)(CC1)C[C@@H]2O)c1cnc(C(F)F)cn1] |
| 353 | 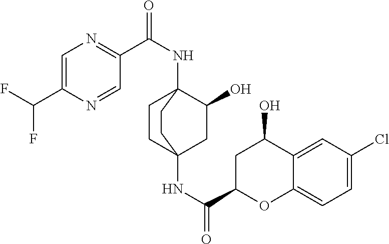 [SMILES: O=C(NC12CCC(NC(=O)[C@H]3C[C@@H](O)c4cc(Cl)ccc4O3)(CC1)C[C@@H]2O)c1cnc(C(F)F)cn1] |
| 354 | 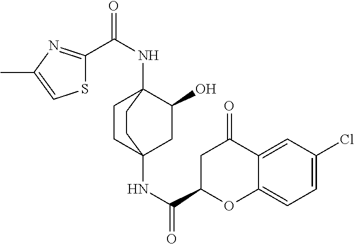 [SMILES: Cc1csc(C(=O)NC23CCC(NC(=O)[C@H]4CC(=O)c5cc(Cl)ccc5O4)(CC2)C[C@@H]3O)n1] |
| 355 | 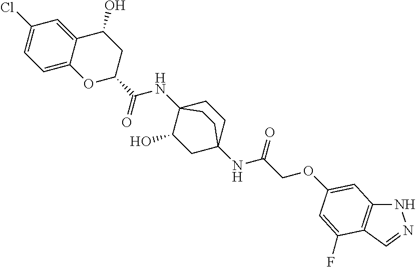 [SMILES: O=C(COc1cc(F)c2cn[nH]c2c1)NC12CCC(NC(=O)[C@H]3C[C@@H](O)c4cc(Cl)ccc4O3)(CC1)[C@@H](O)C2] |
| 356 | 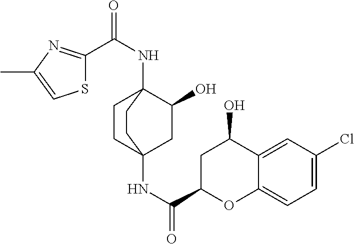 [SMILES: Cc1csc(C(=O)NC23CCC(NC(=O)[C@H]4C[C@@H](O)c5cc(Cl)ccc5O4)(CC2)C[C@@H]3O)n1] |
| 357 | 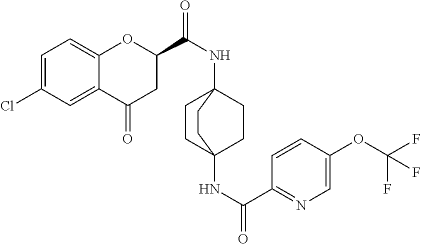 [SMILES: O=C(NC12CCC(NC(=O)[C@H]3CC(=O)c4cc(Cl)ccc4O3)(CC1)CC2)c1ccc(OC(F)(F)F)cn1] |
| 358 | 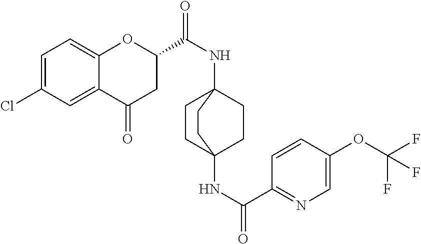 [SMILES: O=C(NC12CCC(NC(=O)[C@@H]3CC(=O)c4cc(Cl)ccc4O3)(CC1)CC2)c1ccc(OC(F)(F)F)cn1] |
| 359 | 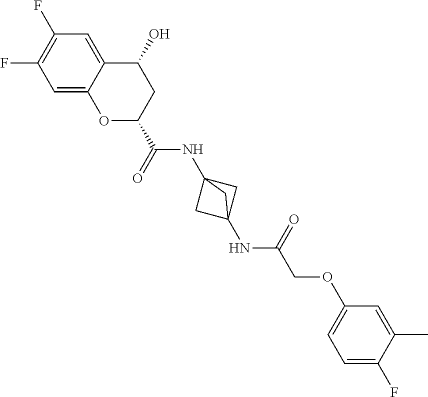 [SMILES: Cc1cc(OCC(=O)NC23CC(NC(=O)[C@H]4C[C@@H](O)c5cc(F)c(F)cc5O4)(C2)C3)ccc1F] |
| 360 | 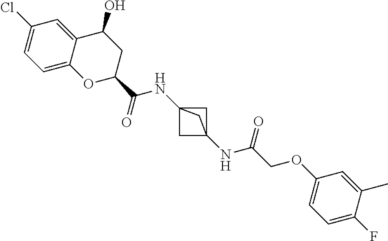 [SMILES: Cc1cc(OCC(=O)NC23CC(NC(=O)[C@@H]4C[C@H](O)c5cc(Cl)ccc5O4)(C2)C3)ccc1F] |
| 361 | 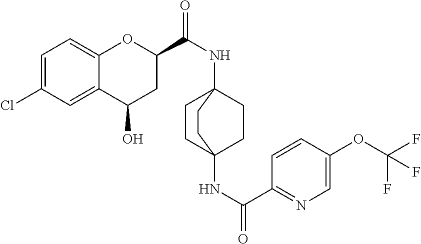 [SMILES: O=C(NC12CCC(NC(=O)[C@H]3C[C@@H](O)c4cc(Cl)ccc4O3)(CC1)CC2)c1ccc(OC(F)(F)F)cn1] |
| 362 | 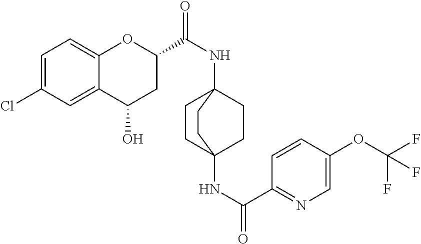 [SMILES: O=C(NC12CCC(NC(=O)[C@@H]3C[C@H](O)c4cc(Cl)ccc4O3)(CC1)CC2)c1ccc(OC(F)(F)F)cn1] |
| 363 | 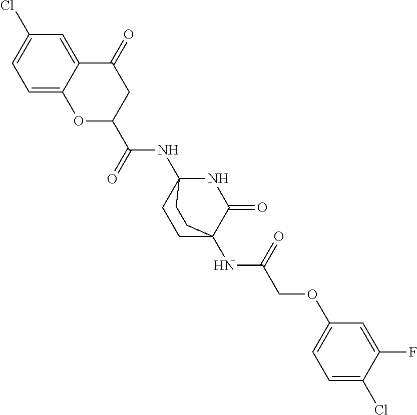 [SMILES: O=C(COc1ccc(Cl)c(F)c1)NC12CCC(NC(=O)C3CC(=O)c4cc(Cl)ccc4O3)(CC1)NC2=O] |
| 364 | 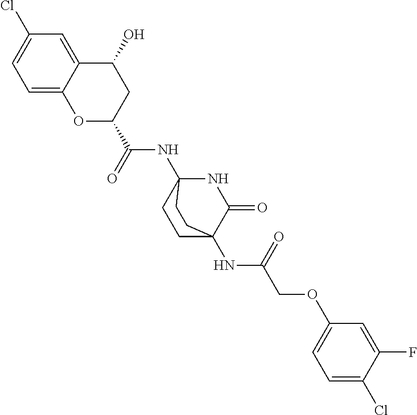 [SMILES: O=C(COc1ccc(Cl)c(F)c1)NC12CCC(NC(=O)[C@H]3C[C@@H](O)c4cc(Cl)ccc4O3)(CC1)NC2=O] |
| 365 | 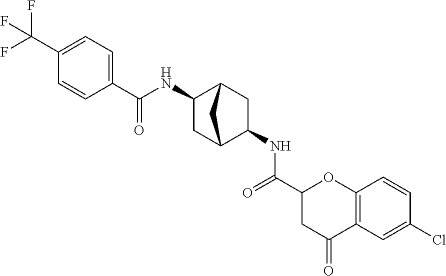 [SMILES: O=C(N[C@@H]1C[C@@H]2C[C@H]1C[C@H]2NC(=O)C1CC(=O)c2cc(Cl)ccc2O1)c1ccc(C(F)(F)F)cc1] |
| 366 | 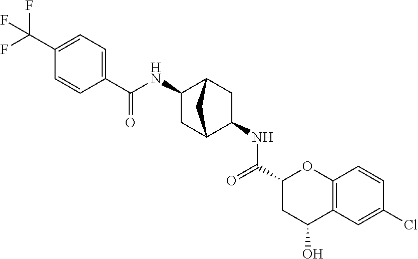 [SMILES: O=C(N[C@@H]1C[C@@H]2C[C@H]1C[C@H]2NC(=O)[C@H]1C[C@@H](O)c2cc(Cl)ccc2O1)c1ccc(C(F)(F)F)cc1] |
| 367 | 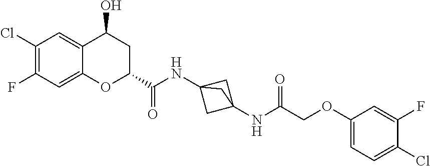 [SMILES: O=C(COc1ccc(Cl)c(F)c1)NC12CC(NC(=O)[C@H]3C[C@H](O)c4cc(Cl)c(F)cc4O3)(C1)C2] |
| 368 | 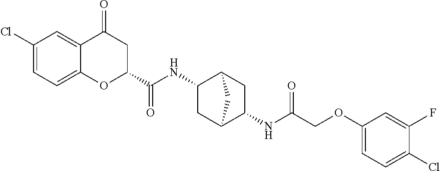 [SMILES: O=C(COc1ccc(Cl)c(F)c1)N[C@H]1C[C@H]2C[C@@H]1C[C@@H]2NC(=O)[C@H]1CC(=O)c2cc(Cl)ccc2O1] |
| 369 | 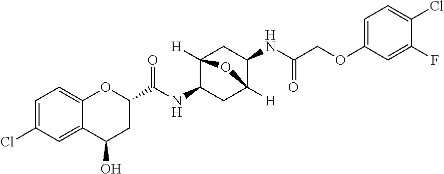 [SMILES: O=C(COc1ccc(Cl)c(F)c1)N[C@@H]1C[C@@H]2O[C@H]1C[C@H]2NC(=O)[C@@H]1C[C@@H](O)c2cc(Cl)ccc2O1] |
| 370 | 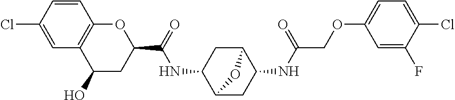 [SMILES: O=C(COc1ccc(Cl)c(F)c1)N[C@@H]1C[C@@H]2O[C@H]1C[C@H]2NC(=O)[C@H]1C[C@@H](O)c2cc(Cl)ccc2O1] |
| 371 | 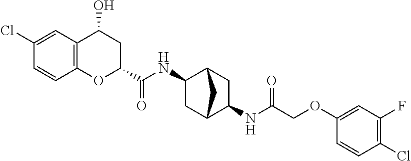 [SMILES: O=C(COc1ccc(Cl)c(F)c1)N[C@@H]1C[C@@H]2C[C@H]1C[C@H]2NC(=O)[C@H]1C[C@@H](O)c2cc(Cl)ccc2O1] |
| 372 | 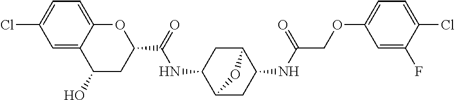 [SMILES: O=C(COc1ccc(Cl)c(F)c1)N[C@@H]1C[C@@H]2O[C@H]1C[C@H]2NC(=O)[C@@H]1C[C@H](O)c2cc(Cl)ccc2O1] |
| 373 | 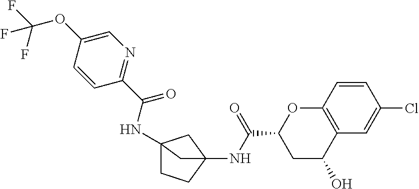 [SMILES: O=C(NC12CCC(NC(=O)[C@H]3C[C@@H](O)c4cc(Cl)ccc4O3)(C1)C2)c1ccc(OC(F)(F)F)cn1] |
| 374 | 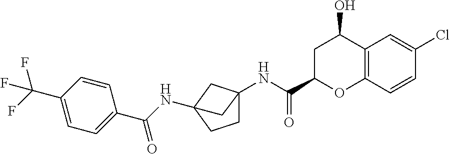 [SMILES: O=C(NC12CCC(NC(=O)[C@H]3C[C@@H](O)c4cc(Cl)ccc4O3)(C1)C2)c1ccc(C(F)(F)F)cc1] |
| 375 | 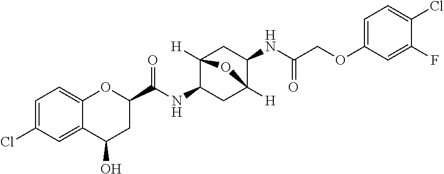 [SMILES: O=C(COc1ccc(Cl)c(F)c1)N[C@@H]1C[C@@H]2O[C@H]1C[C@H]2NC(=O)[C@H]1C[C@@H](O)c2cc(Cl)ccc2O1] |
| 376 | 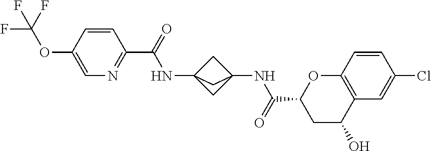 [SMILES: O=C(NC12CC(NC(=O)[C@H]3C[C@@H](O)c4cc(Cl)ccc4O3)(C1)C2)c1ccc(OC(F)(F)F)cn1] |
| 377 | 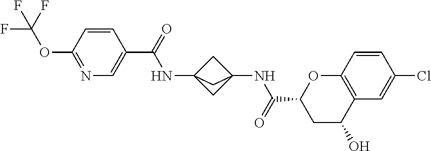 [SMILES: O=C(NC12CC(NC(=O)[C@H]3C[C@@H](O)c4cc(Cl)ccc4O3)(C1)C2)c1ccc(OC(F)(F)F)nc1] |
| 378 | 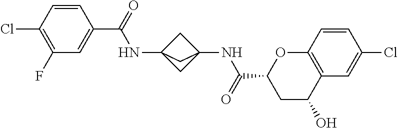 [SMILES: O=C(NC12CC(NC(=O)[C@H]3C[C@@H](O)c4cc(Cl)ccc4O3)(C1)C2)c1ccc(Cl)c(F)c1] |
| 379 | 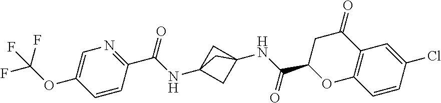 [SMILES: O=C(NC12CC(NC(=O)[C@H]3CC(=O)c4cc(Cl)ccc4O3)(C1)C2)c1ccc(OC(F)(F)F)cn1] |
| 380 | 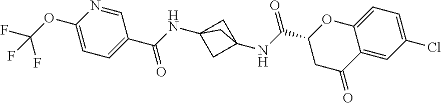 [SMILES: O=C(NC12CC(NC(=O)[C@H]3CC(=O)c4cc(Cl)ccc4O3)(C1)C2)c1ccc(OC(F)(F)F)nc1] |
| 381 | 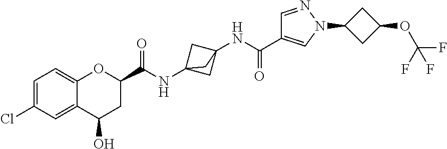 [SMILES: O=C(NC12CC(NC(=O)[C@H]3C[C@@H](O)c4cc(Cl)ccc4O3)(C1)C2)c1cnn([C@H]2C[C@@H](OC(F)(F)F)C2)c1] |
| 382 | 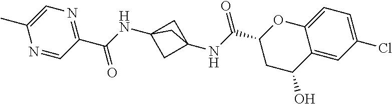 [SMILES: Cc1cnc(C(=O)NC23CC(NC(=O)[C@H]4C[C@@H](O)c5cc(Cl)ccc5O4)(C2)C3)cn1] |
| 383 | 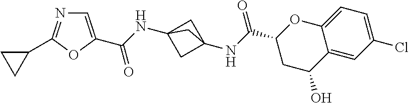 [SMILES: O=C(NC12CC(NC(=O)[C@H]3C[C@@H](O)c4cc(Cl)ccc4O3)(C1)C2)c1cnc(C2CC2)o1] |
| 384 | 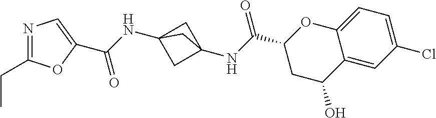 [SMILES: CCc1ncc(C(=O)NC23CC(NC(=O)[C@H]4C[C@@H](O)c5cc(Cl)ccc5O4)(C2)C3)o1] |
| 385 | 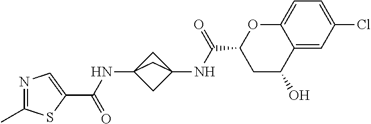 [SMILES: Cc1ncc(C(=O)NC23CC(NC(=O)[C@H]4C[C@@H](O)c5cc(Cl)ccc5O4)(C2)C3)s1] |
| 386 | 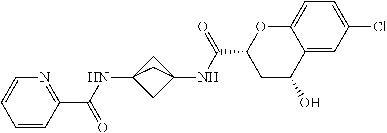 [SMILES: O=C(NC12CC(NC(=O)[C@H]3C[C@@H](O)c4cc(Cl)ccc4O3)(C1)C2)c1ccccn1] |
| 387 | 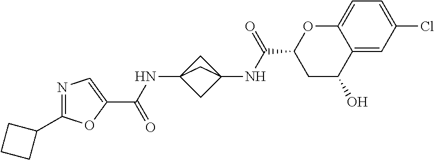 [SMILES: O=C(NC12CC(NC(=O)[C@H]3C[C@@H](O)c4cc(Cl)ccc4O3)(C1)C2)c1cnc(C2CCC2)o1] |
| 388 | 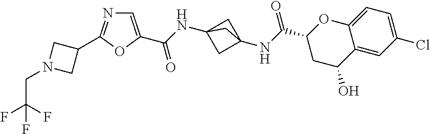 [SMILES: O=C(NC12CC(NC(=O)[C@H]3C[C@@H](O)c4cc(Cl)ccc4O3)(C1)C2)c1cnc(C2CN(CC(F)(F)F)C2)o1] |
| 389 | 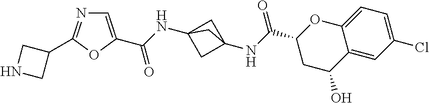 [SMILES: O=C(NC12CC(NC(=O)[C@H]3C[C@@H](O)c4cc(Cl)ccc4O3)(C1)C2)c1cnc(C2CNC2)o1] |
| 390 | 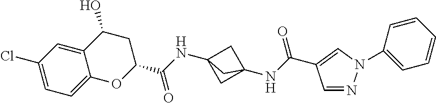 [SMILES: O=C(NC12CC(NC(=O)[C@H]3C[C@@H](O)c4cc(Cl)ccc4O3)(C1)C2)c1cnn(-c2ccccc2)c1] |
| 391 | 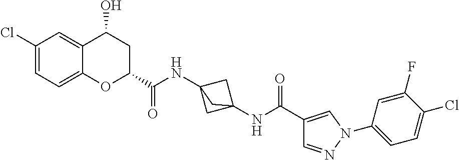 [SMILES: O=C(NC12CC(NC(=O)[C@H]3C[C@@H](O)c4cc(Cl)ccc4O3)(C1)C2)c1cnn(-c2ccc(Cl)c(F)c2)c1] |
| 392 | 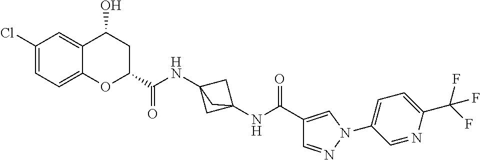 [SMILES: O=C(NC12CC(NC(=O)[C@H]3C[C@@H](O)c4cc(Cl)ccc4O3)(C1)C2)c1cnn(-c2ccc(C(F)(F)F)nc2)c1] |

### Methods of Making Exemplary Compounds

The compounds of the invention may be better understood in connection with the following synthetic schemes and methods which illustrate a means by which the compounds can be prepared. The compounds of this invention can be prepared by a variety of synthetic procedures. Representative synthetic procedures are shown in, but not limited to, Schemes 1-4. The variables A, D, W, L¹, L², R¹, R², R^{B}, R^{C}, R^{W1}, R^{W2}, are defined as detailed herein, e.g., in the Summary.

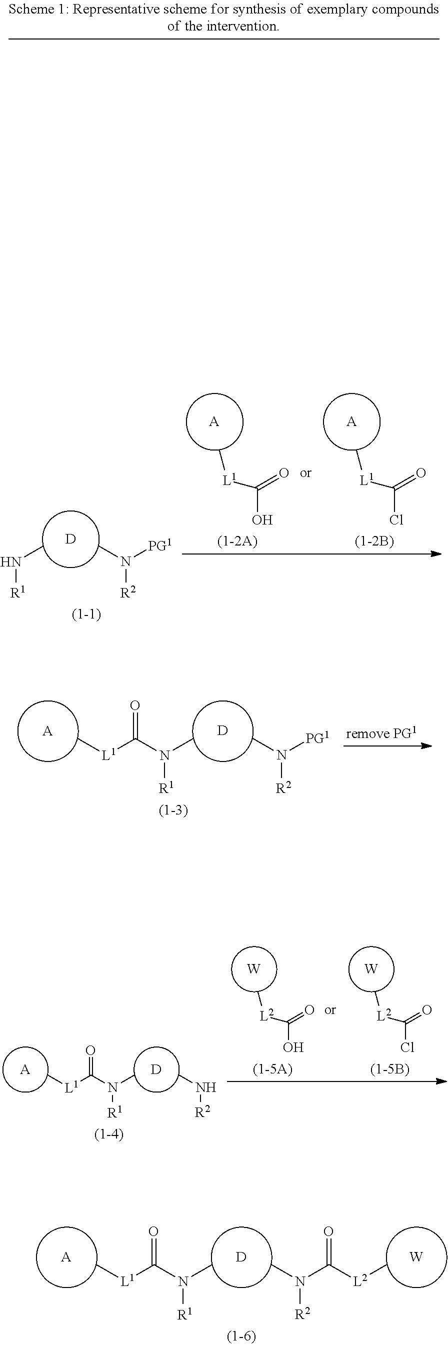

As shown in Scheme 1, compounds of formula (1-6) can be prepared from compounds of formula (1-1). Compounds of formula (1-1) where PG¹ is an amine protecting group (e.g. tert-butoxycarbonyl or benzyloxycarbonyl) can be coupled with carboxylic acids of formula (1-2A) or alternatively with acid chlorides of formula (1-2B) under amide bond forming conditions to give amides of formula (1-3). Examples of conditions known to generate amides from a mixture of a carboxylic acid of formula (1-2A) and an amine of formula (1-1) include but are not limited to adding a coupling reagent such as N-(3-dimethylaminopropyl)-N′-ethylcarbodiimide or 1-(3-dimethylaminopropyl)-3-ethylcarbodiimide (EDC, EDAC or EDCI), 1,3-dicyclohexylcarbodiimide (DCC), bis(2-oxo-3-oxazolidinyl)phosphinic chloride (BOPCl), N-[(dimethylamino)-1H-1,2,3-triazolo-[4,5-b]pyridin-1-ylmethylene]-N-methylmethanaminium hexafluorophosphate N-oxide or 2-(7-azabenzotriazol-1-yl)-N,N,N′,N′-tetramethyluronium hexafluorophosphate or 1-[bis(dimethylamino)methylene]-1H-1,2,3-triazolo[4,5-b]pyridinium 3-oxid hexafluorophosphate or 2-(3H-[1,2,3]triazolo[4,5-b]pyridin-3-yl)-1,1,3,3-tetramethylisouronium hexafluorophosphate (V) or 2-(7-aza-1H-benzotriazole-1-yl)-1,1,3,3-tetramethyluronium hexafluorophosphate (HATU), O-(benzotriazol-1-yl)-N,N,N′,N′-tetramethyluronium tetrafluoroborate (TBTU), 2-(1H-benzo[d][1,2,3]triazol-1-yl)-1,1,3,3-tetramethylisouronium hexafluorophosphate (V) (HBTU), 2,4,6-tripropyl-1,3,5,2,4,6-trioxatriphosphinane 2,4,6-trioxide (T3P®), (1-cyano-2-ethoxy-2-oxoethylidenaminooxy)dimethylamino-morpholino-carbenium hexafluorophosphate (COMU®), and fluoro-N,N,N′,N′-tetramethylformamidinium hexafluorophosphate. The coupling reagents may be added as a solid, a solution, or as the reagent bound to a solid support resin.

In addition to the coupling reagents, auxiliary-coupling reagents may facilitate the coupling reaction. Auxiliary coupling reagents that are often used in the coupling reactions include but are not limited to 4-(dimethylamino)pyridine (DMAP), 1-hydroxy-7-azabenzotriazole (HOAT) and 1-hydroxybenzotriazole (HOBT). The reaction may be carried out optionally in the presence of a base such as triethylamine or diisopropylethylamine. The coupling reaction may be carried out in solvents such as but not limited to tetrahydrofuran, N,N-dimethylformamide, N,N-dimethylacetamide, dimethyl sulfoxide, dichloromethane, and ethyl acetate.

Alternatively, carboxylic acids of formula (1-2A) can be converted to the corresponding acid chlorides of formula (1-2B) by reaction with thionyl chloride, PCl₃, PCl₅, cyanuric chloride, Ghosez's reagent or oxalyl chloride. The reactions with thionyl chloride and oxalyl chloride can be catalyzed with N,N-dimethylformamide at ambient temperature in a solvent such as dichloromethane. The resultant acid chlorides of formula (1-2B) can then be coupled with amines of formula (1-1) optionally in the presence of a base such as a tertiary amine base such as triethylamine or diisopropylethylamine or an aromatic base such as pyridine, at room temperature in a solvent such as dichloromethane to give amides of formula (1-3).

Compounds of formula (1-3) can be deprotected using conditions known to one of skill in the art and dependent upon the protecting group (PG¹) used to give compounds of formula (1-4). Compounds of formula (1-4) can be coupled with carboxylic acids of formula (1-5A) or alternatively acid chlorides of formula (1-5B) under amide bond forming conditions as discussed above to afford compounds of formula (1-6). Compounds of formula (1-6) are representative compounds of Formula (I).

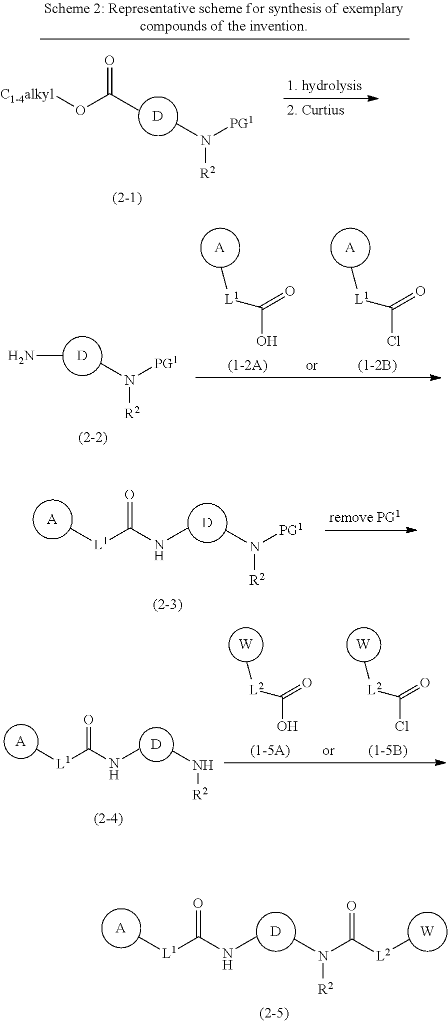

As shown in Scheme 2, compounds of formula (2-5) can be prepared from compounds of formula (2-1). Compounds of formula (2-1) where PG¹ is an amine protecting group (e.g. benzyl, tert-butoxycarbonyl or benzyloxycarbonyl) can be converted to compounds of formula (2-2) in a two-step procedure. In the first step, esters of formula (2-1) can be hydrolyzed to the corresponding carboxylic acids using conditions known to one of skill in the art. In the second step, the carboxylic acids can be treated under Curtius reaction conditions to afford compounds of formula (2-2). Primary amines of formula (2-2) can be coupled with carboxylic acids of formula (1-2A) or alternatively acid chlorides of formula (1-2B) under amide bond forming conditions as disclosed for Scheme 1 to give amides of formula (2-3).

Compounds of formula (2-3) can be deprotected using conditions known to one of skill in the art and dependent upon the protecting group (PG¹) used to give compounds of formula (2-4). Compounds of formula (2-4) can be coupled with carboxylic acids of formula (1-5A) or alternatively acid chlorides of formula (1-5B) under amide bond forming conditions as disclosed for Scheme 1 to afford compounds of formula (2-5). Compounds of formula (2-5) are representative compounds of Formula (I).

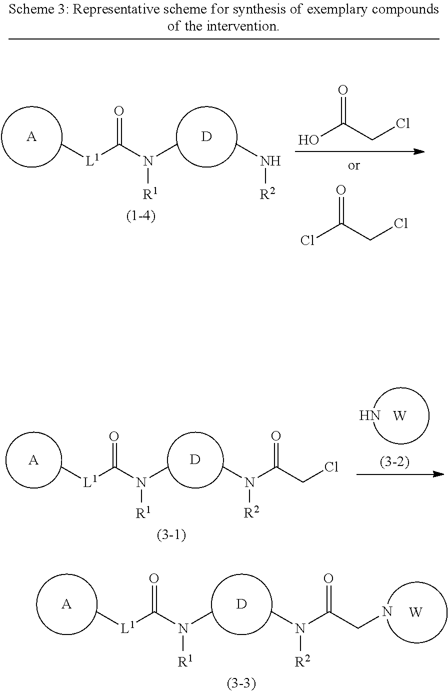

As shown in Scheme 3, compounds of formula (3-3) can be prepared from compounds of formula (1-4). Compounds of formula (1-4) can be coupled with 2-chloroacetic acid or 2-chloroacetyl chloride under the amide bond forming reaction conditions disclosed in Scheme 1 to give compounds of formula (3-1). Compounds of formula (3-1) can be converted to compounds of formula (3-3) under nucleophilic substitution reactions conditions. Compounds of formula (3-1) can be treated with cyclic amines of formula (3-2) in the presence of a base such as potassium carbonate and an activating agent such as potassium iodide with microwave irradiation to afford compounds of formula (3-3). Compounds of formula (3-3) are representative of compounds of formula (I).

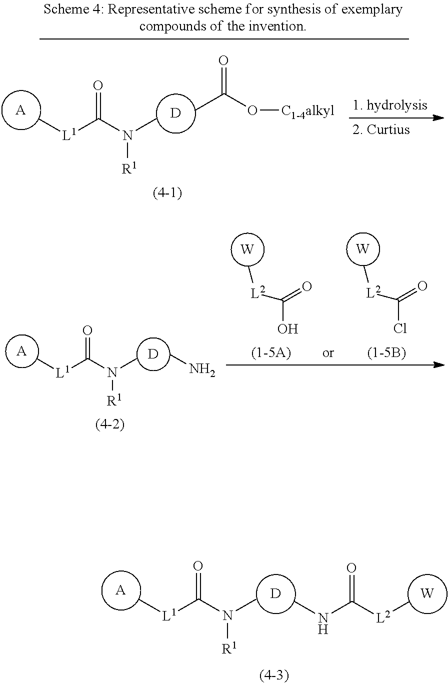

As shown in Scheme 4, compounds of formula (4-3) can be prepared from compounds of formula (4-1). Compounds of formula (4-1) can be converted to compounds of formula (4-2) in a two-step procedure. In the first step, esters of formula (4-1) can be hydrolyzed to the corresponding carboxylic acids using conditions known to one of skill in the art. In the second step, the carboxylic acids can be treated under Curtius reaction conditions to afford compounds of formula (4-2). Primary amines of formula (4-2) can be coupled with carboxylic acids of formula (1-5A) or alternatively acid chlorides of formula (1-5B) under amide bond forming conditions as disclosed for Scheme 1 to give amides of formula (4-3). Compounds of formula (4-3) are representative compounds of Formula (I).

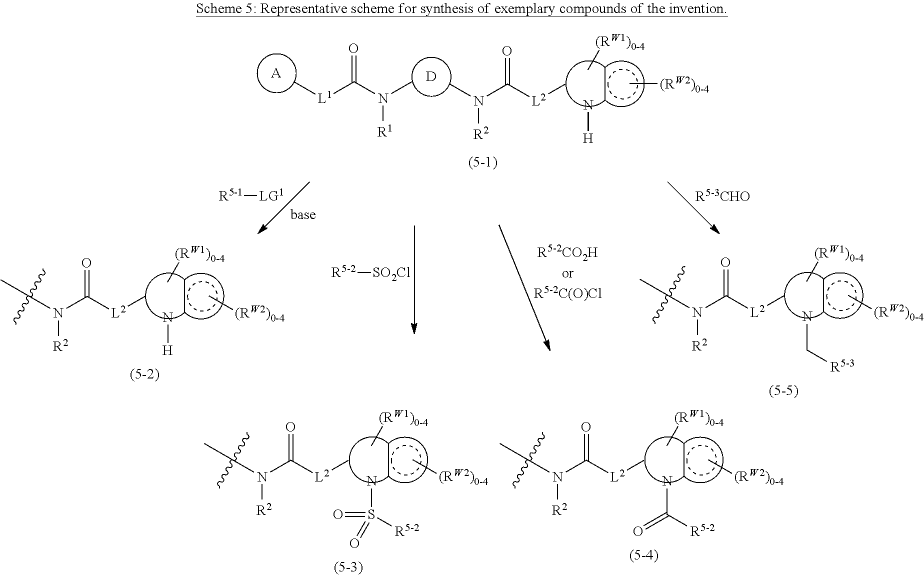

As shown in Scheme 5, compounds of formula (5-2), formula (5-3), formula (5-4) and formula (5-5) can be prepared from compounds of formula (5-1), wherein the fused bicyclic heterocyclyl of formula (5-1) has a substitutable nitrogen moiety. The substitutable nitrogen moiety may be alkylated with an alkylating agent, R⁵⁻¹-LG¹, wherein LG¹ is a halogen or sulfonate and R⁵⁻¹ is an optionally substituted alkyl or haloalkyl, in the presence of a base such as potassium carbonate optionally warmed in a solvent such as but not limited to N,N-dimethylformamide to give compounds of formula (5-2). Compounds of formula (5-1) can be sulfonylated with sulfonyl chlorides, R⁵⁻²—SO₂Cl, wherein R⁵⁻² is an optionally substituted C₁-C₆ alkyl, C₁-C₆ cycloalkyl, phenyl, heterocyclyl or heteroaryl, in the presence of a base such as pyridine or a tertiary amine base in an optionally warmed solvent such as dichloromethane to give sulfonamides of formula (5-3). Compounds of formula (5-1) can be reacted with carboxylic acids, R⁵⁻²—CO₂H, or carboxylic acid chlorides, R⁵⁻²—C(O)C₁, under the conditions described in Scheme 1 to form amides to give amides of formula (5-4). Compounds of formula (5-1) can be reductively aminated under conditions known to one of skill in the art with aldehydes, R⁵⁻³—CHO, wherein R⁵⁻³ is an optionally substituted C₁-C₆ alkyl, to give compounds of formula (5-5). Compounds of formula (5-2), formula (5-3), formula (5-4) and formula (5-5) can be further transformed using methodologies known to one of skill in the art. Compounds of formula (5-2), formula (5-3), formula (5-4) and formula (5-5) are representative of compounds of Formula (I).

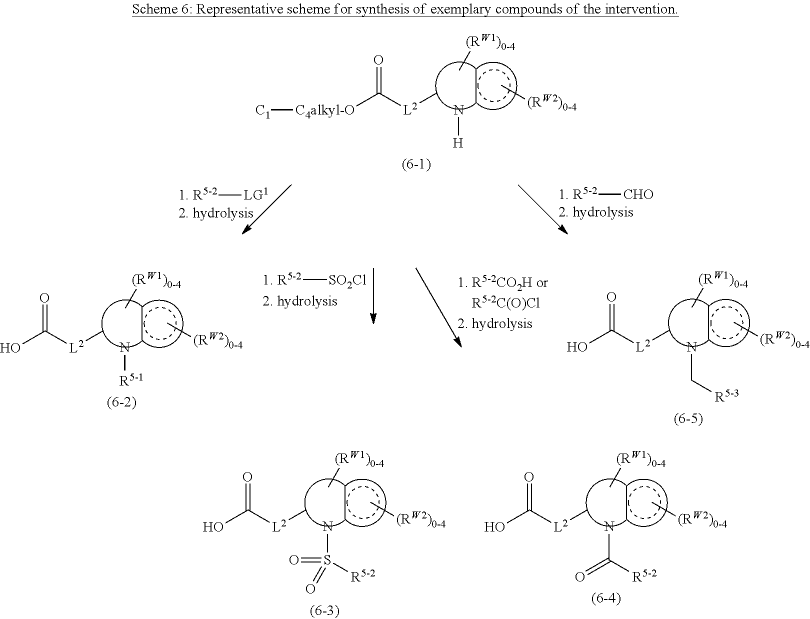

As shown in Scheme 6, compounds of formula (6-2), formula (6-3), formula (6-4) and formula (6-5) can be prepared from compounds of formula (6-1), wherein the fused bicyclic heterocyclyl of formula (6-1) has a substitutable nitrogen moiety. The substitutable nitrogen moiety may be alkylated with an alkylating agent, R⁵⁻¹-LG¹, wherein LG¹ is a halogen or sulfonate and R⁵⁻¹ is an optionally substituted alkyl or haloalkyl, in the presence of a base such as potassium carbonate optionally warmed in a solvent such as but not limited to N,N-dimethylformamide. Subsequent hydrolysis of the ester moiety using methodologies known to one of skill in the art gives compounds of formula (6-2). R⁵⁻¹, R⁵⁻², and R⁵⁻³ are as described in Scheme 5. Compounds of formula (6-1) can be sulfonylated with sulfonyl chlorides, R⁵⁻²—SO₂Cl, in the presence of a base such as pyridine or a tertiary amine base in an optionally warmed solvent such as dichloromethane. Subsequent ester hydrolysis gives sulfonamides of formula (6-3). Compounds of formula (6-1) can be reacted with carboxylic acids, R⁵⁻²—CO₂H, or carboxylic acid chlorides, R⁵⁻²—C(O)Cl, under the conditions described in Scheme 1 to form amides. Subsequent ester hydrolysis gives amides of formula (6-4). Compounds of formula (6-1) can be reductively aminated under conditions known to one of skill in the art with aldehydes, R⁵⁻³—CHO. Subsequent ester hydrolysis gives compounds of formula (6-5). Compounds of formula (6-2), formula (6-3), formula (6-4) and formula (6-5) can be further transformed using methodologies known to one of skill in the art. Compounds of formula (6-2), formula (6-3), formula (6-4) and formula (6-5) can be used as compounds of formula (1-5A) as shown in Schemes 1, 2, and 4.

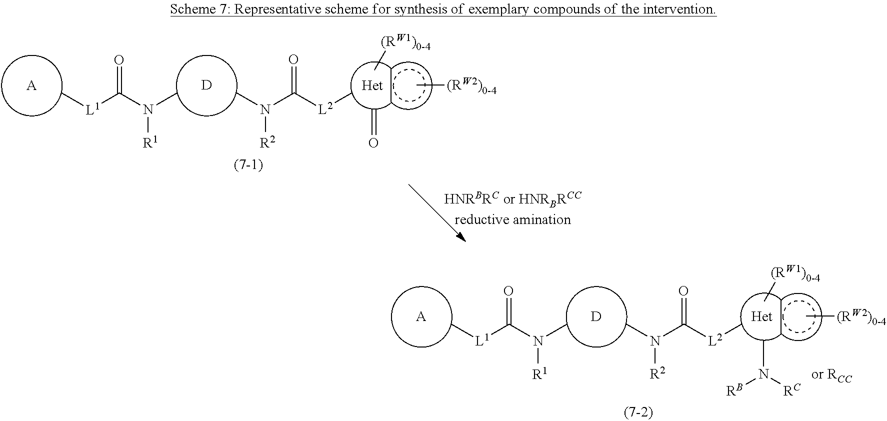

As shown in Scheme 7, compounds of formula (7-2) can be prepared from compounds of formula (7-1) with a reductive amination with HNR^{B}R^{C} or HNR^{B}R^{CC}. Accordingly, compounds of formula (7-1), wherein an oxo group is a substituent on the heterocyclyl portion of W, can be reacted with an amine, HNR^{B}R^{C} or HNR^{B}R^{CC}, under reductive amination conditions. Such conditions can be but are not limited to combining compounds of formula (7-1) and HNR^{B}R^{C} or HNR^{B}R^{CC} in a solvent such as methanol in the presence of an acid such as zinc chloride with subsequent treatment with a reductant such as sodium cyanoborohydride to give compounds of formula (7-2). Compounds of formula (7-2) are representative of compounds of Formula (I).

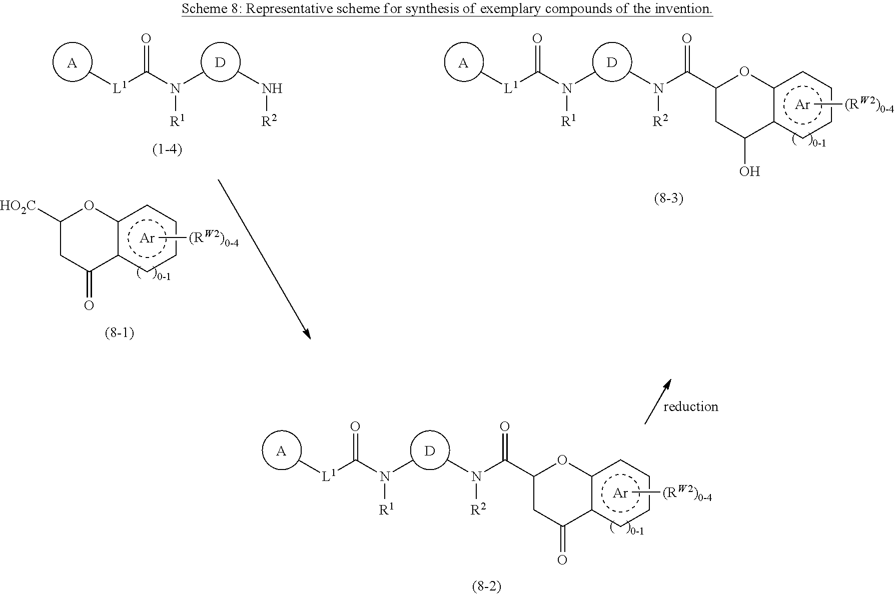

As shown in Scheme 8, compounds of formula (8-3) can be prepared from compounds of formula (1-4). Compounds of formula (1-4) can be coupled with compounds of formula (8-1), wherein Ar is a fused aryl or heteroaryl ring, under amide bond forming conditions described in Scheme 1 to give compounds of formula (8-2). Compounds of formula (8-2) can be reduced to compounds of formula (8-3) using a reductant such as sodium cyanoborohydride in the presence of zinc chloride in an optionally warmed solvent such as methanol or sodium borohydride in a solvent such as methanol. Compounds of formula (8-3) are representative of compounds of Formula (I).

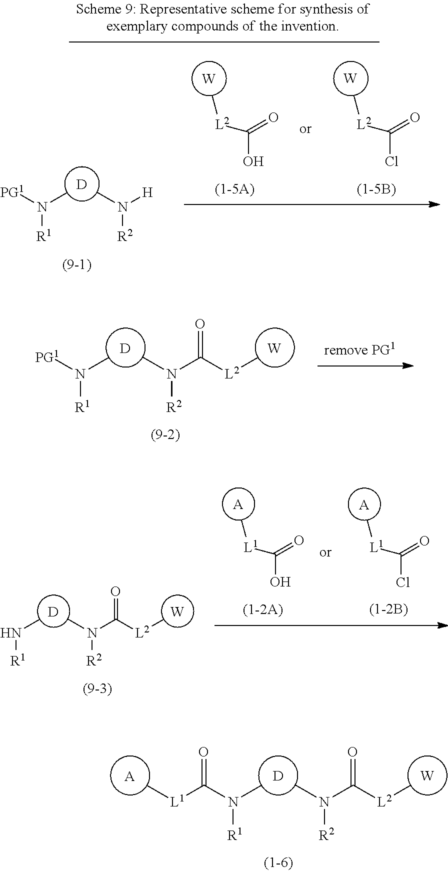

As shown in Scheme 9, compounds of formula (1-6) can be prepared from compounds of formula (9-1). Compounds of formula (9-1), wherein PG¹ is an amine protecting group (e.g. tert-butoxycarbonyl or benzyloxycarbonyl) can be coupled with carboxylic acids of formula (1-5A) or alternatively with acid chlorides of formula (1-5B) under amide bond forming conditions described in Scheme 1 to give amides of formula (9-2). Compounds of formula (9-2) can be deprotected using conditions known to one of skill in the art and dependent upon the protecting group (PG¹) used to give compounds of formula (9-3). Compounds of formula (9-3) can be coupled with carboxylic acids of formula (1-2A) or alternatively acid chlorides of formula (1-2B) under amide bond forming conditions as discussed above to afford compounds of formula (1-6). Compounds of formula (1-6) are representative compounds of Formula (I).

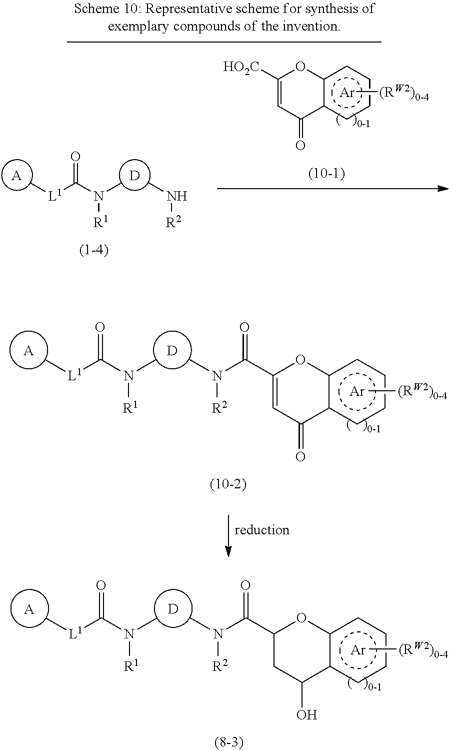

As shown in Scheme 10, compounds of formula (8-3) can be prepared from compounds of formula (1-4). Compounds of formula (1-4) can be coupled with compounds of formula (10-1), wherein Ar is a fused aryl or heteroaryl ring, under amide bond forming conditions described in Scheme 1 to give compounds of formula (10-2). Compounds of formula (10-2) can be reduced to compounds of formula (8-3) using a reductant such as sodium borohydride in an optionally warmed solvent such as methanol. Compounds of formula (10-2) and compounds of formula (8-3) are representative of compounds of Formula (I).

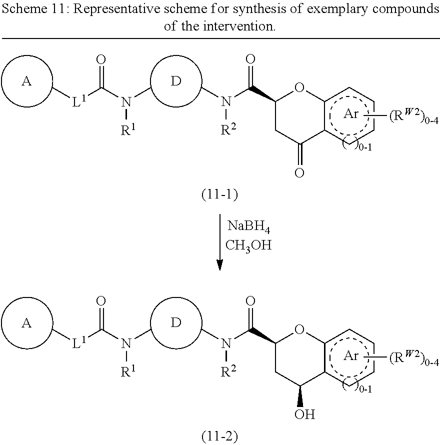

As shown in Scheme 11, compounds of formula (11-2) can be prepared from compounds of formula (11-1). Compounds of formula (11-1), wherein Ar is a fused aryl or heteroaryl ring, can be reduced to compounds of formula (11-2) using a reductant such as sodium borohydride in an optionally warmed solvent such as methanol. Compounds of formula (11-2) are representative of compounds of Formula (I).

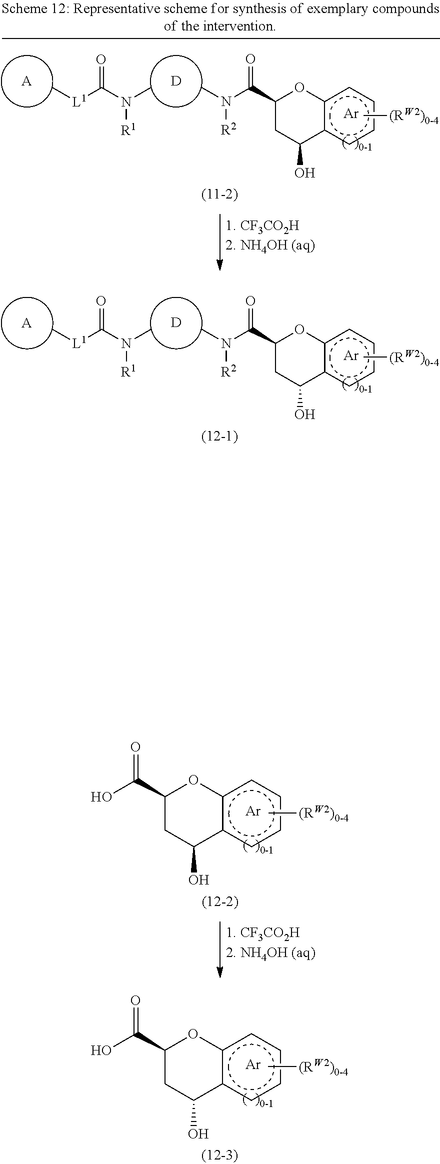

As shown in Scheme 12, compounds of formula (12-1) can be prepared from compounds of formula (11-2). Compounds of formula (11-2), wherein Ar is a fused aryl or heteroaryl ring, can be converted to compounds of formula (12-1) by treatment with optionally warmed trifluoroacetic acid for 0.5-4 hours followed by aqueous ammonium hydroxide. Similarly, compounds of formula (12-2) can be transformed to compounds of formula (12-3) under the same conditions. Compounds of formula (12-3) are intermediates to prepare compounds of Formula (I). Compounds of formula (12-1) are representative of compounds of Formula (I).

### Pharmaceutical Compositions

The present invention features pharmaceutical compositions comprising a compound of Formula (I) or a pharmaceutically acceptable salt, solvate, hydrate, tautomer, ester, N-oxide or stereoisomer thereof. In some embodiments, the pharmaceutical composition further comprises a pharmaceutically acceptable excipient. In some embodiments, the compound of Formula (I) or a pharmaceutically acceptable salt, solvate, hydrate, tautomer, stereoisomer thereof is provided in an effective amount in the pharmaceutical composition. In some embodiments, the effective amount is a therapeutically effective amount. In certain embodiments, the effective amount is a prophylactically effective amount.

Pharmaceutical compositions described herein can be prepared by any method known in the art of pharmacology. In general, such preparatory methods include the steps of bringing the compound of Formula (I) (the “active ingredient”) into association with a carrier and/or one or more other accessory ingredients, and then, if necessary and/or desirable, shaping and/or packaging the product into a desired single- or multi-dose unit. Pharmaceutical compositions can be prepared, packaged, and/or sold in bulk, as a single unit dose, and/or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of a compound of Formula (I), the pharmaceutically acceptable excipient, and/or any additional ingredients in a pharmaceutical composition of the invention will vary, depending upon the identity, size, and/or condition of the subject treated and further depending upon the route by which the composition is to be administered. By way of example, the composition may comprise between 0.1% and 100% (w/w) of a compound of Formula (I).

The term “pharmaceutically acceptable excipient” refers to a non-toxic carrier, adjuvant, diluent, or vehicle that does not destroy the pharmacological activity of the compound with which it is formulated. Pharmaceutically acceptable excipients useful in the manufacture of the pharmaceutical compositions of the invention are any of those that are well known in the art of pharmaceutical formulation and include inert diluents, dispersing and/or granulating agents, surface active agents and/or emulsifiers, disintegrating agents, binding agents, preservatives, buffering agents, lubricating agents, and/or oils. Pharmaceutically acceptable excipients useful in the manufacture of the pharmaceutical compositions of the invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Compositions of the present invention may be administered orally, parenterally (including subcutaneous, intramuscular, intravenous and intradermal), by inhalation spray, topically, rectally, nasally, buccally, vaginally or via an implanted reservoir. In some embodiments, provided compounds or compositions are administrable intravenously and/or orally.

The term “parenteral” as used herein includes subcutaneous, intravenous, intramuscular, intraocular, intravitreal, intra-articular, intra-synovial, intrasternal, intrathecal, intrahepatic, intraperitoneal intralesional and intracranial injection or infusion techniques. Preferably, the compositions are administered orally, subcutaneously, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

Pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added. In some embodiments, a provided oral formulation is formulated for immediate release or sustained/delayed release. In some embodiments, the composition is suitable for buccal or sublingual administration, including tablets, lozenges and pastilles. A compound of Formula (I) may also be in micro-encapsulated form.

The compositions of the present invention can be delivered by transdermally, by a topical route, formulated as applicator sticks, solutions, suspensions, emulsions, gels, creams, ointments, pastes, jellies, paints, powders, and aerosols. Oral preparations include tablets, pills, powder, dragees, capsules, liquids, lozenges, cachets, gels, syrups, slurries, suspensions, etc., suitable for ingestion by the patient. Solid form preparations include powders, tablets, pills, capsules, cachets, suppositories, and dispersible granules. Liquid form preparations include solutions, suspensions, and emulsions, for example, water or water/propylene glycol solutions. The compositions of the present invention may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions of the present invention can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). In another embodiment, the formulations of the compositions of the present invention can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn, Curr. Opin. Biotechnol. 6:698-708, 1995; Ostro, J. Hosp. Pharm. 46: 1576-1587, 1989). The compositions of the present invention can also be delivered as nanoparticles.

Alternatively, pharmaceutically acceptable compositions of this invention may be administered in the form of suppositories for rectal administration. Pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

In some embodiments, in order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Compounds provided herein, e.g., a compound of Formula (I) or a pharmaceutically acceptable salt, solvate, hydrate, tautomer, ester, N-oxide or stereoisomer thereof are typically formulated in dosage unit form, e.g., single unit dosage form, for ease of administration and uniformity of dosage. It will be understood, however, that the total daily usage of the compositions of the present invention will be decided by the attending physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex and diet of the subject; the time of administration, route of administration, and rate of excretion of the specific active ingredient employed; the duration of the treatment; drugs used in combination or coincidental with the specific active ingredient employed; and like factors well known in the medical arts.

The exact amount of a compound required to achieve an effective amount will vary from subject to subject, depending, for example, on species, age, and general condition of a subject, severity of the side effects or disorder, identity of the particular compound(s), mode of administration, and the like. The desired dosage can be delivered three times a day, two times a day, once a day, every other day, every third day, every week, every two weeks, every three weeks, or every four weeks. In certain embodiments, the desired dosage can be delivered using multiple administrations (e.g., two, three, four, five, six, seven, eight, nine, ten, eleven, twelve, thirteen, fourteen, or more administrations).

In certain embodiments, an effective amount of a compound of Formula (I) or a pharmaceutically acceptable salt, solvate, hydrate, tautomer, ester, N-oxide or stereoisomer thereof for administration one or more times a day may comprise about 0.0001 mg to about 5000 mg, e.g., from about 0.0001 mg to about 4000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of a compound per unit dosage form.

In certain embodiments, a compound of Formula (I) or a pharmaceutically acceptable salt, solvate, hydrate, tautomer, ester, N-oxide or stereoisomer thereof may be at dosage levels sufficient to deliver from about 0.001 mg/kg to about 1000 mg/kg, e.g., about 0.001 mg/kg to about 500 mg/kg, about 0.01 mg/kg to about 250 mg/kg, about 0.1 mg/kg to about 100 mg/kg, about 0.1 mg/kg to about 50 mg/kg, about 0.1 mg/kg to about 40 mg/kg, about 0.1 mg/kg to about 25 mg/kg, about 0.01 mg/kg to about 10 mg/kg, about 0.1 mg/kg to about 10 mg/kg, or about 1 mg/kg to about 50 mg/kg, of subject body weight per day, one or more times a day, to obtain the desired therapeutic effect.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

It will be also appreciated that a compound or composition, e.g., a compound of Formula (I) or a pharmaceutically acceptable salt, solvate, hydrate, tautomer, ester, N-oxide or stereoisomer thereof as described herein, can be administered in combination with one or more additional pharmaceutical agents. The compounds or compositions can be administered in combination with additional pharmaceutical agents that improve their bioavailability, reduce and/or modify their metabolism, inhibit their excretion, and/or modify their distribution within the body. It will also be appreciated that the therapy employed may achieve a desired effect for the same disorder, and/or it may achieve different effects.

The compound or composition can be administered concurrently with, prior to, or subsequent to, one or more additional pharmaceutical agents, which may be useful as, e.g., combination therapies. Pharmaceutical agents include therapeutically active agents. Pharmaceutical agents also include prophylactically active agents. Each additional pharmaceutical agent may be administered at a dose and/or on a time schedule determined for that pharmaceutical agent. The additional pharmaceutical agents may also be administered together with each other and/or with the compound or composition described herein in a single dose or administered separately in different doses. The particular combination to employ in a regimen will take into account compatibility of the inventive compound with the additional pharmaceutical agents and/or the desired therapeutic and/or prophylactic effect to be achieved. In general, it is expected that the additional pharmaceutical agents utilized in combination be utilized at levels that do not exceed the levels at which they are utilized individually. In some embodiments, the levels utilized in combination will be lower than those utilized individually.

Exemplary additional pharmaceutical agents include, but are not limited to, anti-proliferative agents, anti-cancer agents, anti-diabetic agents, anti-inflammatory agents, immunosuppressant agents, and pain-relieving agents. Pharmaceutical agents include small organic molecules such as drug compounds (e.g., compounds approved by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells.

Pharmaceutical compositions provided by the present invention include compositions wherein the active ingredient (e.g., compounds described herein, including embodiments or examples) is contained in a therapeutically effective amount, i.e., in an amount effective to achieve its intended purpose. The actual amount effective for a particular application will depend, inter alia, on the condition being treated. When administered in methods to treat a disease, such compositions will contain an amount of active ingredient effective to achieve the desired result, e.g., modulating the activity of a target molecule (e.g. eIF2B, eIF2 or component of eIF2α signal transduction pathway or component of phosphorylated eIF2α pathway or the ISR pathway), and/or reducing, eliminating, or slowing the progression of disease symptoms (e.g. symptoms of cancer a neurodegenerative disease, a leukodystrophy, an inflammatory disease, a musculoskeletal disease, a metabolic disease, or a disease or disorder associated with impaired function of eIF2B, eIF2α or a component of the eIF2 pathway or ISR pathway). Determination of a therapeutically effective amount of a compound of the invention is well within the capabilities of those skilled in the art, especially in light of the detailed disclosure herein.

The dosage and frequency (single or multiple doses) administered to a mammal can vary depending upon a variety of factors, for example, whether the mammal suffers from another disease, and its route of administration; size, age, sex, health, body weight, body mass index, and diet of the recipient; nature and extent of symptoms of the disease being treated (e.g. a symptom of cancer, a neurodegenerative disease, a leukodystrophy, an inflammatory disease, a musculoskeletal disease, a metabolic disease, or a disease or disorder associated with impaired function of eIF2B, eIF2 α, or a component of the eIF2 pathway or ISR pathway), kind of concurrent treatment, complications from the disease being treated or other health-related problems. Other therapeutic regimens or agents can be used in conjunction with the methods and compounds of Applicants' invention. Adjustment and manipulation of established dosages (e.g., frequency and duration) are well within the ability of those skilled in the art.

For any compound described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

Dosages may be varied depending upon the requirements of the patient and the compound being employed. The dose administered to a patient, in the context of the present invention should be sufficient to affect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. Dosage amounts and intervals can be adjusted individually to provide levels of the administered compound effective for the particular clinical indication being treated. This will provide a therapeutic regimen that is commensurate with the severity of the individual's disease state.

Utilizing the teachings provided herein, an effective prophylactic or therapeutic treatment regimen can be planned that does not cause substantial toxicity and yet is effective to treat the clinical symptoms demonstrated by the particular patient. This planning should involve the careful choice of active compound by considering factors such as compound potency, relative bioavailability, patient body weight, presence and severity of adverse side effects, preferred mode of administration and the toxicity profile of the selected agent.

Also encompassed by the invention are kits (e.g., pharmaceutical packs). The inventive kits may be useful for preventing and/or treating a disease (e.g., cancer, a neurodegenerative disease, a leukodystrophy, an inflammatory disease, a musculoskeletal disease, a metabolic disease, or other disease or condition described herein).

The kits provided may comprise an inventive pharmaceutical composition or compound and a container (e.g., a vial, ampule, bottle, syringe, and/or dispenser package, or other suitable container). In some embodiments, provided kits may optionally further include a second container comprising a pharmaceutical excipient for dilution or suspension of an inventive pharmaceutical composition or compound. In some embodiments, the inventive pharmaceutical composition or compound provided in the container and the second container are combined to form one unit dosage form.

Thus, in one aspect, provided are kits including a first container comprising a compound of Formula (I) or a pharmaceutically acceptable salt, solvate, hydrate, tautomer, ester, N-oxide or stereoisomer thereof, or a pharmaceutical composition thereof. In certain embodiments, the kits are useful in preventing and/or treating a proliferative disease in a subject. In certain embodiments, the kits further include instructions for administering a compound of Formula (I) or a pharmaceutically acceptable salt, solvate, hydrate, tautomer, ester, N-oxide or stereoisomer thereof, or a pharmaceutical composition thereof, to a subject to prevent and/or treat a disease described herein.

### Methods of Treatment

The present invention features compounds, compositions, and methods comprising a compound of Formula (I), or a pharmaceutically acceptable salt, solvate, hydrate, tautomer, ester, N-oxide or stereoisomer thereof. In some embodiments, the compounds, compositions, and methods are used in the prevention or treatment of a disease, disorder, or condition. Exemplary diseases, disorders, or conditions include, but are not limited to a neurodegenerative disease, a leukodystrophy, a cancer, an inflammatory disease, an autoimmune disease, a viral infection, a skin disease, a fibrotic disease, a hemoglobin disease, a kidney disease, a hearing loss condition, an ocular disease, a disease with mutations that leads to UPR induction, a malaria infection, a musculoskeletal disease, a metabolic disease, or a mitochondrial disease.

In some embodiments, the disease, disorder, or condition is related to (e.g., caused by) modulation of (e.g., a decrease in) eIF2B activity or level, eIF2α activity or level, or a component of the eIF2 pathway or ISR pathway. In some embodiments, the disease, disorder, or condition is related to modulation of a signaling pathway related to a component of the eIF2 pathway or ISR pathway (e.g., phosphorylation of a component of the eIF2 pathway or ISR pathway). In some embodiments, the disease, disorder, or condition is related to (e.g., caused by) neurodegeneration. In some embodiments, the disease, disorder, or condition is related to (e.g., caused by) neural cell death or dysfunction. In some embodiments, the disease, disorder, or condition is related to (e.g., caused by) glial cell death or dysfunction. In some embodiments, the disease, disorder, or condition is related to (e.g., caused by) an increase in the level or activity of eIF2B, eIF2α, or a component of the eIF2 pathway or ISR pathway. In some embodiments, the disease, disorder, or condition is related to (e.g., caused by) a decrease in the level or activity of eIF2B, eIF2α, or a component of the eIF2 pathway or ISR pathway.

In some embodiments, the disease may be caused by a mutation to a gene or protein sequence related to a member of the eIF2 pathway (e.g., eIF2B, eIF2α, or other component). Exemplary mutations include an amino acid mutation in the eIF2B1, eIF2B2, eIF2B3, eIF2B4, eIF2B5 subunits. In some embodiments, an amino acid mutation (e.g., an amino acid substitution, addition, or deletion) in a particular protein that may result in a structural change, e.g., a conformational or steric change, that affects the function of the protein. For example, in some embodiments, amino acids in and around the active site or close to a binding site (e.g., a phosphorylation site, small molecule binding site, or protein-binding site) may be mutated such that the activity of the protein is impacted. In some instances, the amino acid mutation (e.g., an amino acid substitution, addition, or deletion) may be conservative and may not substantially impact the structure or function of a protein. For example, in certain cases, the substitution of a serine residue with a threonine residue may not significantly impact the function of a protein. In other cases, the amino acid mutation may be more dramatic, such as the substitution of a charged amino acid (e.g., aspartic acid or lysine) with a large, nonpolar amino acid (e.g., phenylalanine or tryptophan) and therefore may have a substantial impact on protein function. The nature of the mutations that affect the structure of function of a gene or protein may be readily identified using standard sequencing techniques, e.g., deep sequencing techniques that are well known in the art. In some embodiments, a mutation in a member of the eIF2 pathway may affect binding or activity of a compound of Formula (I), or a pharmaceutically acceptable salt, solvate, hydrate, tautomer, ester, N-oxide or stereoisomer thereof and thereby modulate treatment of a particular disease, disorder, or condition, or a symptom thereof.

In some embodiments, an eIF2 protein may comprise an amino acid mutation (e.g., an amino acid substitution, addition, or deletion) at an alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, or valine residue. In some embodiments, an eIF2 protein may comprise an amino acid substitution at an alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, or valine residue. In some embodiments, an eIF2 protein may comprise an amino acid addition at an alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, or valine residue. In some embodiments, an eIF2 protein may comprise an amino acid deletion at an alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, or valine residue.

In some embodiments, the eIF2 protein may comprise an amino acid mutation (e.g., an amino acid substitution, addition, or deletion) at an alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, or valine residue in the eIF2B1, eIF2B2, eIF2B3, eIF2B4, eIF2B5 subunits. In some embodiments, the eIF2 protein may comprise an amino acid substitution at an alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, or valine residue in the eIF2B1, eIF2B2, eIF2B3, eIF2B4, eIF2B5 subunits. In some embodiments, the eIF2 protein may comprise an amino acid addition at an alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, or valine residue in the eIF2B1, eIF2B2, eIF2B3, eIF2B4, eIF2B5 subunits. In some embodiments, the eIF2 protein may comprise an amino acid deletion at an alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, or valine residue in the eIF2B1, eIF2B2, eIF2B3, eIF2B4, eIF2B5 subunits. Exemplary mutations include V183F (eIF2B1 subunit), H341Q (eIF2B3), I346T (eIF2B3), R483W (eIF2B4), R113H (eIF2B5), and R195H (eIF2B5).

In some embodiments, an amino acid mutation (e.g., an amino acid substitution, addition, or deletion) in a member of the eIF2 pathway (e.g., an eIF2B protein subunit) may affect binding or activity of a compound of Formula (I), or a pharmaceutically acceptable salt, solvate, hydrate, tautomer, ester, N-oxide or stereoisomer thereof and thereby modulate treatment of a particular disease, disorder, or condition, or a symptom thereof.

### Neurodegenerative Disease

In some embodiments, the compound of Formula (I), or a pharmaceutically acceptable salt, solvate, hydrate, tautomer, ester, N-oxide or stereoisomer thereof is used to treat a neurodegenerative disease. As used herein, the term “neurodegenerative disease” refers to a disease or condition in which the function of a subject's nervous system becomes impaired. Examples of a neurodegenerative disease that may be treated with a compound, pharmaceutical composition, or method described herein include Alexander's disease, Alper's disease, Alzheimer's disease, Amyotrophic lateral sclerosis (ALS), Ataxia telangiectasia, Batten disease (also known as Spielmeyer-Vogt-Sjogren-Batten disease), Bovine spongiform encephalopathy (BSE), Canavan disease, Cockayne syndrome, Corticobasal degeneration, Creutzfeldt-Jakob disease, Dystonia, frontotemporal dementia (FTD), Gerstmann-Straussler-Scheinker syndrome, Huntington's disease, HIV-associated dementia, Kennedy's disease, Krabbe disease, kuru, Lewy body dementia, Machado-Joseph disease (Spinocerebellar ataxia type 3), Multiple system atrophy, Multisystem proteinopathy, Narcolepsy, Neuroborreliosis, Parkinson's disease, Pelizaeus-Merzbacher Disease, Pick's disease, Primary lateral sclerosis, Prion diseases, Refsum's disease, Sandhoff disease, Schilder's disease, Subacute combined degeneration of spinal cord secondary to Pernicious Anaemia, Schizophrenia, Spinocerebellar ataxia (multiple types with varying characteristics, e.g., Spinocerebellar ataxia type 2 or Spinocerebellar ataxia type 8), Spinal muscular atrophy, Steele-Richardson-Olszewski disease, progressive supranuclear palsy, corticobasal degeneration, adrenoleukodystrophy, X-linked adrenoleukodystrophy, cerebral adrenoleukodystrophy, Pelizaeus-Merzbacher Disease, Krabbe disease, leukodystrophy due to mutation in DARS2 gene (sometimes known as lukoencephalopathy with brainstem and spinal cord involvement and lactate elevation (LBSL), DARS2-related spectrum disorders, or Tabes dorsalis.

In some embodiments, the neurodegenerative disease comprises vanishing white matter disease, childhood ataxia with CNS hypo-myelination, a leukodystrophy, a leukoencephalopathy, a hypomyelinating or demyelinating disease, an intellectual disability syndrome (e.g., Fragile X syndrome), Alzheimer's disease, amyotrophic lateral sclerosis (ALS), Creutzfeldt-Jakob disease, frontotemporal dementia (FTD), Gerstmann-Straussler-Scheinker disease, Huntington's disease, dementia (e.g., HIV-associated dementia or Lewy body dementia), kuru, multiple sclerosis, Parkinson's disease, or a prion disease.

In some embodiments, the neurodegenerative disease comprises vanishing white matter disease, childhood ataxia with CNS hypo-myelination, a leukodystrophy, a leukoencephalopathy, a hypomyelinating or demyelinating disease, or an intellectual disability syndrome (e.g., Fragile X syndrome).

In some embodiments, the neurodegenerative disease comprises a psychiatric disease such as agoraphobia, Alzheimer's disease, anorexia nervosa, amnesia, anxiety disorder, attention deficit disorder, bipolar disorder, body dysmorphic disorder, bulimia nervosa, claustrophobia, depression, delusions, Diogenes syndrome, dyspraxia, insomnia, Munchausen's syndrome, narcolepsy, narcissistic personality disorder, obsessive-compulsive disorder, psychosis, phobic disorder, schizophrenia, seasonal affective disorder, schizoid personality disorder, sleepwalking, social phobia, substance abuse, tardive dyskinesia, Tourette syndrome, or trichotillomania.

In some embodiments, the compound of Formula (I), or a pharmaceutically acceptable salt, solvate, hydrate, tautomer, ester, N-oxide or stereoisomer thereof is used to treat vanishing white matter disease. Exemplary methods of treating vanishing white matter disease include, but are not limited to, reducing or eliminating a symptom of vanishing white matter disease, reducing the loss of white matter, reducing the loss of myelin, increasing the amount of myelin, or increasing the amount of white matter in a subject.

In some embodiments, the compound of Formula (I), or a pharmaceutically acceptable salt, solvate, hydrate, tautomer, ester, N-oxide or stereoisomer thereof is used to treat childhood ataxia with CNS hypo-myelination. Exemplary methods of treating childhood ataxia with CNS hypo-myelination include, but are not limited to, reducing or eliminating a symptom of childhood ataxia with CNS hypo-myelination, increasing the level of myelin, or decreasing the loss of myelin in a subject.

In some embodiments, the compound of Formula (I), or a pharmaceutically acceptable salt, solvate, hydrate, tautomer, ester, N-oxide or stereoisomer thereof is used to treat an intellectual disability syndrome (e.g., Fragile X syndrome). Exemplary methods of treating an intellectual disability syndrome include, but are not limited to, reducing or eliminating a symptom of an intellectual disability syndrome.

In some embodiments, the compound of Formula (I), or a pharmaceutically acceptable salt, solvate, hydrate, tautomer, ester, N-oxide or stereoisomer thereof is used to treat neurodegeneration. Exemplary methods of treating neurodegeneration include, but are not limited to, improvement of mental wellbeing, increasing mental function, slowing the decrease of mental function, decreasing dementia, delaying the onset of dementia, improving cognitive skills, decreasing the loss of cognitive skills, improving memory, decreasing the degradation of memory, or extending survival.

In some embodiments, the compound of Formula (I), or a pharmaceutically acceptable salt, solvate, hydrate, tautomer, ester, N-oxide or stereoisomer thereof is used to treat a leukoencephalopathy or demyelinating disease. Exemplary leukoencephalopathies include, but are not limited to, progressive multifocal leukoencephalopathy, toxic leukoencephalopathy, leukoencephalopathy with vanishing white matter, leukoencephalopathy with neuroaxonal spheroids, reversible posterior leukoencephalopathy syndrome, hypertensive leukoencephalopathy, megalencephalic leukoencephalopathy with subcortical cysts, Charcot-Marie-Tooth disorder, and Devic's disease. A leukoencephalopathy may comprise a demyelinating disease, which may be inherited or acquired. In some embodiments, an acquired demyelinating disease may be an inflammatory demyelinating disease (e.g., an infectious inflammatory demyelinating disease or a non-infectious inflammatory demyelinating disease), a toxic demyelinating disease, a metabolic demyelinating disease, a hypoxic demyelinating disease, a traumatic demyelinating disease, or an ischemic demyelinating disease (e.g., Binswanger's disease). Exemplary methods of treating a leukoencephalopathy or demyelinating disease include, but are not limited to, reducing or eliminating a symptom of a leukoencephalopathy or demyelinating disease, reducing the loss of myelin, increasing the amount of myelin, reducing the loss of white matter in a subject, or increasing the amount of white matter in a subject.

In some embodiments, the compound of Formula (I), or a pharmaceutically acceptable salt, solvate, hydrate, tautomer, ester, N-oxide or stereoisomer thereof is used to treat a traumatic injury or a toxin-induced injury to the nervous system (e.g., the brain). Exemplary traumatic brain injuries include, but are not limited to, a brain abscess, concussion, ischemia, brain bleeding, cranial fracture, diffuse axonal injury, locked-in syndrome, or injury relating to a traumatic force or blow to the nervous system or brain that causes damage to an organ or tissue. Exemplary toxin-induced brain injuries include, but are not limited to, toxic encephalopathy, meningitis (e.g. bacterial meningitis or viral meningitis), meningoencephalitis, encephalitis (e.g., Japanese encephalitis, eastern equine encephalitis, West Nile encephalitis), Guillan-Barre syndrome, Sydenham's chorea, rabies, leprosy, neurosyphilis, a prion disease, or exposure to a chemical (e.g., arsenic, lead, toluene, ethanol, manganese, fluoride, dichlorodiphenyltrichloroethane (DDT), dichlorodiphenyldichloroethylene (DDE), tetrachloroethylene, a polybrominated diphenyl ether, a pesticide, a sodium channel inhibitor, a potassium channel inhibitor, a chloride channel inhibitor, a calcium channel inhibitor, or a blood brain barrier inhibitor).

In other embodiments, the compound of Formula (I), or a pharmaceutically acceptable salt, solvate, hydrate, tautomer, ester, N-oxide or stereoisomer thereof is used to improve memory in a subject. Induction of memory has been shown to be facilitated by decreased and impaired by increased eIF2α phosphorylation. Regulators of translation, such as compounds disclosed herein (e.g. a compound of Formula (I)), could serve as therapeutic agents that improve memory in human disorders associated with memory loss such as Alzheimer's disease and in other neurological disorders that activate the UPR or ISR in neurons and thus could have negative effects on memory consolidation such as Parkinson's disease, schizophrenia, amyotrophic lateral sclerosis (ALS) and prion diseases. In addition, a mutation in eIF2γ that disrupts complex integrity linked intellectual disability (intellectual disability syndrome or ID) to impaired translation initiation in humans. Hence, two diseases with impaired eIF2 function, ID and VWM, display distinct phenotypes but both affect mainly the brain and impair learning. In some embodiments, the disease or condition is unsatisfactory memory (e.g., working memory, long term memory, short term memory, or memory consolidation).

In still other embodiments, the compound of Formula (I), or a pharmaceutically acceptable salt, solvate, hydrate, tautomer, ester, N-oxide or stereoisomer thereof is used in a method to improve memory in a subject (e.g., working memory, long term memory, short term memory, or memory consolidation). In some embodiments, the subject is human. In some embodiments, the subject is a non-human mammal. In some embodiments, the subject is a domesticated animal. In some embodiments, the subject is a dog. In some embodiments, the subject is a bird. In some embodiments, the subject is a horse. In embodiments, the patient is a bovine. In some embodiments, the subject is a primate.

### Cancer

In some embodiments, the compound of Formula (I), or a pharmaceutically acceptable salt, solvate, hydrate, tautomer, ester, N-oxide or stereoisomer thereof is used to treat cancer. As used herein, “cancer” refers to human cancers and carcinomas, sarcomas, adenocarcinomas, lymphomas, leukemias, melanomas, etc., including solid and lymphoid cancers, kidney, breast, lung, bladder, colon, ovarian, prostate, pancreas, stomach, brain, head and neck, skin, uterine, testicular, glioma, esophagus, liver cancer, including hepatocarcinoma, lymphoma, including B-acute lymphoblastic lymphoma, non-Hodgkin's lymphomas (e.g., Burkitt's, Small Cell, and Large Cell lymphomas), Hodgkin's lymphoma, leukemia (including AML, ALL, and CML), and/or multiple myeloma. In some further instances, “cancer” refers to lung cancer, breast cancer, ovarian cancer, leukemia, lymphoma, melanoma, pancreatic cancer, sarcoma, bladder cancer, bone cancer, brain cancer, cervical cancer, colon cancer, esophageal cancer, gastric cancer, liver cancer, head and neck cancer, kidney cancer, myeloma, thyroid cancer, prostate cancer, metastatic cancer, or carcinoma.

As used herein, the term “cancer” refers to all types of cancer, neoplasm or malignant tumors found in mammals, including leukemia, lymphoma, carcinomas and sarcomas. Exemplary cancers that may be treated with a compound, pharmaceutical composition, or method provided herein include lymphoma, sarcoma, bladder cancer, bone cancer, brain tumor, cervical cancer, colon cancer, esophageal cancer, gastric cancer, head and neck cancer, kidney cancer, myeloma, thyroid cancer, leukemia, prostate cancer, breast cancer (e.g., ER positive, ER negative, chemotherapy resistant, herceptin resistant, HER2 positive, doxorubicin resistant, tamoxifen resistant, ductal carcinoma, lobular carcinoma, primary, metastatic), ovarian cancer, pancreatic cancer, liver cancer (e.g., hepatocellular carcinoma), lung cancer (e.g., non-small cell lung carcinoma, squamous cell lung carcinoma, adenocarcinoma, large cell lung carcinoma, small cell lung carcinoma, carcinoid, sarcoma), glioblastoma multiforme, glioma, or melanoma. Additional examples include, cancer of the thyroid, endocrine system, brain, breast, cervix, colon, head & neck, liver, kidney, lung, non-small cell lung, melanoma, mesothelioma, ovary, sarcoma, stomach, uterus or Medulloblastoma (e.g., WNT-dependent pediatric medulloblastoma), Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, Paget's Disease of the Nipple, Phyllodes Tumors, Lobular Carcinoma, Ductal Carcinoma, cancer of the pancreatic stellate cells, cancer of the hepatic stellate cells, or prostate cancer.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound, pharmaceutical composition, or method provided herein include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocyte leukemia, micromyeloblastic leukemia, monocytic leukemia, myeloblasts leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

The term “sarcoma” generally refers to a tumor which is made up of a substance like the embryonic connective tissue and is generally composed of closely packed cells embedded in a fibrillar or homogeneous substance. Sarcomas that may be treated with a compound, pharmaceutical composition, or method provided herein include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” is taken to mean a tumor arising from the melanocytic system of the skin and other organs. Melanomas that may be treated with a compound, pharmaceutical composition, or method provided herein include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, or superficial spreading melanoma.

The term “carcinoma” refers to a malignant new growth made up of epithelial cells tending to infiltrate the surrounding tissues and give rise to metastases. Exemplary carcinomas that may be treated with a compound, pharmaceutical composition, or method provided herein include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, ductal carcinoma, carcinoma durum, embryonal carcinoma, encephaloid carcinoma, epidermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lobular carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tubular carcinoma, tuberous carcinoma, verrucous carcinoma, or carcinoma villosum.

In some embodiments, the compound of Formula (I) or a pharmaceutically acceptable salt, solvate, hydrate, tautomer, ester, N-oxide or stereoisomer thereof is used to treat pancreatic cancer, breast cancer, multiple myeloma, cancers of secretory cells. For example certain methods herein treat cancer by decreasing or reducing or preventing the occurrence, growth, metastasis, or progression of cancer. In some embodiments, the methods described herein may be used to treat cancer by decreasing or eliminating a symptom of cancer. In some embodiments, the compound of Formula (I) or a pharmaceutically acceptable salt, solvate, hydrate, tautomer, ester, N-oxide or stereoisomer thereof may be used as a single agent in a composition or in combination with another agent in a composition to treat a cancer described herein (e.g., pancreatic cancer, breast cancer, multiple myeloma, cancers of secretory cells).

In some embodiments, the compounds (compounds described herein, e.g., a compound of Formula (I)) and compositions (e.g., compositions comprising a compound described herein, e.g., a compound of Formula (I))) are used with a cancer immunotherapy (e.g., a checkpoint blocking antibody) to treat a subject (e.g., a human subject), e.g., suffering from a disease or disorder described herein (e.g., abnormal cell growth, e.g., cancer (e.g., a cancer described herein)). The methods described herein comprise administering a compound described herein, e.g., a compound of Formula (I) and an immunotherapy to a subject having abnormal cell growth such as cancer. Exemplary immunotherapies include, but are not limited to the following.

In some embodiments, the immunotherapeutic agent is a compound (e.g., a ligand, an antibody) that inhibits the immune checkpoint blockade pathway. In some embodiments, the immunotherapeutic agent is a compound that inhibits the indoleamine 2,3-dioxygenase (IDO) pathway. In some embodiments, the immunotherapeutic agent is a compound that agonizes the STING pathway. Cancer immunotherapy refers to the use of the immune system to treat cancer. Three groups of immunotherapy used to treat cancer include cell-based, antibody-based, and cytokine therapies. All groups exploit cancer cells' display of subtly different structures (e.g., molecular structure; antigens, proteins, molecules, carbohydrates) on their surface that can be detected by the immune system. Cancer immunotherapy (i.e., anti-tumor immunotherapy or anti-tumor immunotherapeutics) includes but is not limited to, immune checkpoint antibodies (e.g., PD-1 antibodies, PD-L1 antibodies, PD-L2 antibodies, CTLA-4 antibodies, TIM3 antibodies, LAG3 antibodies, TIGIT antibodies); and cancer vaccines (i.e., anti-tumor vaccines or vaccines based on neoantigens such as a peptide or RNA vaccine).

Cell-based therapies (e.g., cancer vaccines), usually involve the removal of immune cells from a subject suffering from cancer, either from the blood or from a tumor. Immune cells specific for the tumor will be activated, grown, and returned to a subject suffering from cancer where the immune cells provide an immune response against the cancer. Cell types that can be used in this way are e.g., natural killer cells, lymphokine-activated killer cells, cytotoxic T-cells, dendritic cells, CAR-T therapies (i.e., chimeric antigen receptor T-cells which are T-cells engineered to target specific antigens), TIL therapy (i.e., administration of tumor-infiltrating lymphocytes), TCR gene therapy, protein vaccines, and nucleic acid vaccines. An exemplary cell-based therapy is Provenge. In some embodiments, the cell-based therapy is a CAR-T therapy.

Interleukin-2 and interferon-alpha are examples of cytokines, proteins that regulate and coordinate the behavior of the immune system.

Cancer Vaccines with Neoantigens

Neoantigens are antigens encoded by tumor-specific mutated genes. Technological innovations have made it possible to dissect the immune response to patient-specific neoantigens that arise as a consequence of tumor-specific mutations, and emerging data suggest that recognition of such neoantigens is a major factor in the activity of clinical immunotherapies. These observations indicate that neoantigen load may form a biomarker in cancer immunotherapy. Many novel therapeutic approaches are being developed that selectively enhance T cell reactivity against this class of antigens. One approach to target neoantigens is via cancer vaccine. These vaccines can be developed using peptides or RNA, e.g., synthetic peptides or synthetic RNA.

Antibody therapies are antibody proteins produced by the immune system and that bind to a target antigen on the surface of a cell. Antibodies are typically encoded by an immunoglobulin gene or genes, or fragments thereof. In normal physiology antibodies are used by the immune system to fight pathogens. Each antibody is specific to one or a few proteins, and those that bind to cancer antigens are used, e.g., for the treatment of cancer. Antibodies are capable of specifically binding an antigen or epitope. (Fundamental Immunology, 3^{rd} Edition, W. E., Paul, ed., Raven Press, N.Y. (1993). Specific binding occurs to the corresponding antigen or epitope even in the presence of a heterogeneous population of proteins and other biologics. Specific binding of an antibody indicates that it binds to its target antigen or epitope with an affinity that is substantially greater than binding to irrelevant antigens. The relative difference in affinity is often at least 25% greater, more often at least 50% greater, most often at least 100% greater. The relative difference can be at least 2-fold, at least 5-fold, at least 10-fold, at least 25-fold, at least 50-fold, at least 100-fold, or at least 1000-fold, for example.

Exemplary types of antibodies include without limitation human, humanized, chimeric, monoclonal, polyclonal, single chain, antibody binding fragments, and diabodies. Once bound to a cancer antigen, antibodies can induce antibody-dependent cell-mediated cytotoxicity, activate the complement system, prevent a receptor interacting with its ligand or deliver a payload of chemotherapy or radiation, all of which can lead to cell death. Exemplary antibodies for the treatment of cancer include but are not limited to, Alemtuzumab, Bevacizumab, Bretuximab vedotin, Cetuximab, Gemtuzumab ozogamicin, Ibritumomab tiuxetan, Ipilimumab, Ofatumumab, Panitumumab, Rituximab, Tositumomab, Trastuzumab, Nivolumab, Pembrolizumab, Avelumab, durvalumab and pidilizumab.

### Checkpoint Blocking Antibodies

The methods described herein comprise, in some embodiments, treating a human subject suffering from a disease or disorder described herein, the method comprising administering a composition comprising a cancer immunotherapy (e.g., an immunotherapeutic agent). In some embodiments, the immunotherapeutic agent is a compound (e.g., an inhibitor or antibody) that inhibits the immune checkpoint blockade pathway. Immune checkpoint proteins, under normal physiological conditions, maintain self-tolerance (e.g., prevent autoimmunity) and protect tissues from damage when the immune system is responding to e.g., pathogenic infection. Immune checkpoint proteins can be dysregulated by tumors as an important immune resistance mechanism. (Pardoll, Nature Rev. Cancer, 2012, 12, 252-264). Agonists of co-stimulatory receptors or antagonists of inhibitory signals (e.g., immune checkpoint proteins), provide an amplification of antigen-specific T-cell responses. Antibodies that block immune checkpoints do not target tumor cells directly but typically target lymphocyte receptors or their ligands to enhance endogenous antitumor activity.

Exemplary checkpoint blocking antibodies include but are not limited to, anti-CTLA-4, anti-PD-1, anti-LAG3 (i.e., antibodies against lymphocyte activation gene 3), and anti-TIM3 (i.e., antibodies against T-cell membrane protein 3). Exemplary anti-CTLA-4 antibodies include but are not limited to, ipilimumab and tremelimumab. Exemplary anti-PD-1 ligands include but are not limited to, PD-L1 (i.e., B7-H1 and CD274) and PD-L2 (i.e., B7-DC and CD273). Exemplary anti-PD-1 antibodies include but are not limited to, nivolumab (i.e., MDX-1106, BMS-936558, or ONO-4538)), CT-011, AMP-224, pembrolizumab (trade name Keytruda), and MK-3475. Exemplary PD-L1-specific antibodies include but are not limited to, BMS936559 (i.e., MDX-1105), MEDI4736 and MPDL-3280A. Exemplary checkpoint blocking antibodies also include but are not limited to, IMP321 and MGA271.

T-regulatory cells (e.g., CD4+, CD25+, or T-reg) are also involved in policing the distinction between self and non-self (e.g., foreign) antigens, and may represent an important mechanism in suppression of immune response in many cancers. T-reg cells can either emerge from the thymus (i.e., “natural T-reg”) or can differentiate from mature T-cells under circumstances of peripheral tolerance induction (i.e., “induced T-reg”). Strategies that minimize the action of T-reg cells would therefore be expected to facilitate the immune response to tumors. (Sutmuller, van Duivernvoorde et al., 2001).

### IDO Pathway Inhibitors

The IDO pathway regulates immune response by suppressing T cell function and enabling local tumor immune escape. IDO expression by antigen-presenting cells (APCs) can lead to tryptophan depletion, and resulting antigen-specific T cell energy and regulatory T cell recruitment. Some tumors even express IDO to shield themselves from the immune system. A compound that inhibits IDO or the IDO pathway thereby activating the immune system to attack the cancer (e.g., tumor in a subject). Exemplary IDO pathway inhibitors include indoximod, epacadostat and EOS200271.

### STING Pathway Agonists

Stimulator of interferon genes (STING) is an adaptor protein that plays an important role in the activation of type I interferons in response to cytosolic nucleic acid ligands. Evidence indicates involvement of the STING pathway in the induction of antitumor immune response. It has been shown that activation of the STING-dependent pathway in cancer cells can result in tumor infiltration with immune cells and modulation of the anticancer immune response. STING agonists are being developed as a class of cancer therapeutics. Exemplary STING agonists include MK-1454 and ADU-S100.

### Co-Stimulatory Antibodies

The methods described herein comprise, in some embodiments, treating a human subject suffering from a disease or disorder described herein, the method comprising administering a composition comprising a cancer immunotherapy (e.g., an immunotherapeutic agent). In some embodiments, the immunotherapeutic agent is a co-stimulatory inhibitor or antibody. In some embodiments, the methods described herein comprise depleting or activating anti-4-1BB, anti-OX40, anti-GITR, anti-CD27 and anti-CD40, and variants thereof.

Inventive methods of the present invention contemplate single as well as multiple administrations of a therapeutically effective amount of a compound as described herein. Compounds, e.g., a compound as described herein, can be administered at regular intervals, depending on the nature, severity and extent of the subject's condition. In some embodiments, a compound described herein is administered in a single dose. In some embodiments, a compound described herein is administered in multiple doses.

### Inflammatory Disease

In some embodiments, the compound of Formula (I), or a pharmaceutically acceptable salt, solvate, hydrate, tautomer, ester, N-oxide or stereoisomer thereof is used to treat an inflammatory disease. As used herein, the term “inflammatory disease” refers to a disease or condition characterized by aberrant inflammation (e.g. an increased level of inflammation compared to a control such as a healthy person not suffering from a disease). Examples of inflammatory diseases include postoperative cognitive dysfunction, arthritis (e.g., rheumatoid arthritis, psoriatic arthritis, juvenile idiopathic arthritis), systemic lupus erythematosus (SLE), myasthenia gravis, juvenile onset diabetes, diabetes mellitus type 1, Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, Behcet's disease, Crohn's disease, ulcerative colitis, bullous pemphigoid, sarcoidosis, ichthyosis, Graves' ophthalmopathy, inflammatory bowel disease, Addison's disease, Vitiligo, asthma (e.g., allergic asthma), acne vulgaris, celiac disease, chronic prostatitis, inflammatory bowel disease, pelvic inflammatory disease, reperfusion injury, sarcoidosis, transplant rejection, interstitial cystitis, atherosclerosis, and atopic dermatitis. Proteins associated with inflammation and inflammatory diseases (e.g. aberrant expression being a symptom or cause or marker of the disease) include interleukin-6 (IL-6), interleukin-8 (IL-8), interleukin-18 (IL-18), TNF-a (tumor necrosis factor-alpha), and C-reactive protein (CRP).

In some embodiments, the inflammatory disease comprises postoperative cognitive dysfunction, arthritis (e.g., rheumatoid arthritis, psoriatic arthritis, or juvenile idiopathic arthritis), systemic lupus erythematosus (SLE), myasthenia gravis, diabetes (e.g., juvenile onset diabetes or diabetes mellitus type 1), Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, Behcet's disease, Crohn's disease, ulcerative colitis, bullous pemphigoid, sarcoidosis, ichthyosis, Graves' ophthalmopathy, inflammatory bowel disease, Addison's disease, vitiligo, asthma (e.g., allergic asthma), acne vulgaris, celiac disease, chronic prostatitis, pelvic inflammatory disease, reperfusion injury, sarcoidosis, transplant rejection, interstitial cystitis, atherosclerosis, or atopic dermatitis.

In some embodiments, the inflammatory disease comprises postoperative cognitive dysfunction, which refers to a decline in cognitive function (e.g. memory or executive function (e.g. working memory, reasoning, task flexibility, speed of processing, or problem solving)) following surgery.

In other embodiments, the method of treatment is a method of prevention. For example, a method of treating postsurgical cognitive dysfunction may include preventing postsurgical cognitive dysfunction or a symptom of postsurgical cognitive dysfunction or reducing the severity of a symptom of postsurgical cognitive dysfunction by administering a compound described herein prior to surgery.

In some embodiments, the compound of Formula (I), or a pharmaceutically acceptable salt, solvate, hydrate, tautomer, ester, N-oxide or stereoisomer thereof is used to treat an inflammatory disease (e.g., an inflammatory disease described herein) by decreasing or eliminating a symptom of the disease. In some embodiments, the compound of Formula (I), or a pharmaceutically acceptable salt, solvate, hydrate, tautomer, ester, N-oxide or stereoisomer thereof may be used as a single agent in a composition or in combination with another agent in a composition to treat an inflammatory disease (e.g., an inflammatory disease described herein).

### Musculoskeletal Diseases

In some embodiments, the compound of Formula (I), or a pharmaceutically acceptable salt, solvate, hydrate, tautomer, ester, N-oxide or stereoisomer thereof is used to treat a musculoskeletal disease. As used herein, the term “musculoskeletal disease” refers to a disease or condition in which the function of a subject's musculoskeletal system (e.g., muscles, ligaments, tendons, cartilage, or bones) becomes impaired. Exemplary musculoskeletal diseases that may be treated with a compound of Formula (I), or a pharmaceutically acceptable salt, solvate, hydrate, tautomer, ester, N-oxide or stereoisomer thereof include muscular dystrophy (e.g., Duchenne muscular dystrophy, Becker muscular dystrophy, distal muscular dystrophy, congenital muscular dystrophy, Emery-Dreifuss muscular dystrophy, facioscapulohumeral muscular dystrophy, myotonic muscular dystrophy type 1, or myotonic muscular dystrophy type 2), limb girdle muscular dystrophy, multisystem proteinopathy, rhizomelic chondrodysplasia punctata, X-linked recessive chondrodysplasia punctata, Conradi-Hünermann syndrome, Autosomal dominant chondrodysplasia punctata, stress induced skeletal disorders (e.g., stress induced osteoporosis), multiple sclerosis, amyotrophic lateral sclerosis (ALS), primary lateral sclerosis, progressive muscular atrophy, progressive bulbar palsy, pseudobulbar palsy, spinal muscular atrophy, progressive spinobulbar muscular atrophy, spinal cord spasticity, spinal muscle atrophy, myasthenia gravis, neuralgia, fibromyalgia, Machado-Joseph disease, Paget's disease of bone, cramp fasciculation syndrome, Freidrich's ataxia, a muscle wasting disorder (e.g., muscle atrophy, sarcopenia, cachexia), an inclusion body myopathy, motor neuron disease, or paralysis.

In some embodiments, the compound of Formula (I), or a pharmaceutically acceptable salt, solvate, hydrate, tautomer, ester, N-oxide or stereoisomer thereof is used to treat a musculoskeletal disease (e.g., a musculoskeletal disease described herein) by decreasing or eliminating a symptom of the disease. In some embodiments, the method of treatment comprises treatment of muscle pain or muscle stiffness associated with a musculoskeletal disease. In some embodiments, the compound of Formula (I), or a pharmaceutically acceptable salt, solvate, hydrate, tautomer, ester, N-oxide or stereoisomer thereof may be used as a single agent in a composition or in combination with another agent in a composition to treat a musculoskeletal disease (e.g., a musculoskeletal disease described herein).

### Metabolic Diseases

In some embodiments, the compound of Formula (I), or a pharmaceutically acceptable salt, solvate, hydrate, tautomer, ester, N-oxide or stereoisomer thereof is used to treat metabolic disease. As used herein, the term “metabolic disease” refers to a disease or condition affecting a metabolic process in a subject. Exemplary metabolic diseases that may be treated with a compound of Formula (I), or a pharmaceutically acceptable salt, solvate, hydrate, tautomer, ester, N-oxide or stereoisomer thereof include non-alcoholic steatohepatitis (NASH), non-alcoholic fatty liver disease (NAFLD), liver fibrosis, obesity, heart disease, atherosclerosis, arthritis, cystinosis, diabetes (e.g., Type I diabetes, Type II diabetes, or gestational diabetes), phenylketonuria, proliferative retinopathy, or Kearns-Sayre disease.

In some embodiments, the compound of Formula (I), or a pharmaceutically acceptable salt, solvate, hydrate, tautomer, ester, N-oxide or stereoisomer thereof is used to treat a metabolic disease (e.g., a metabolic disease described herein) by decreasing or eliminating a symptom of the disease. In some embodiments, the method of treatment comprises decreasing or eliminating a symptom comprising elevated blood pressure, elevated blood sugar level, weight gain, fatigue, blurred vision, abdominal pain, flatulence, constipation, diarrhea, jaundice, and the like. In some embodiments, the compound of Formula (I), or a pharmaceutically acceptable salt, solvate, hydrate, tautomer, ester, N-oxide or stereoisomer thereof may be used as a single agent in a composition or in combination with another agent in a composition to treat a metabolic disease (e.g., a musculoskeletal disease described herein).

### Mitochondrial Diseases

In some embodiments, the compound of Formula (I) or a pharmaceutically acceptable salt, solvate, hydrate, tautomer, ester, N-oxide or stereoisomer thereof is used to treat mitochondrial disease. As used herein, the term “mitochondrial disease” refers to a disease or condition affecting the mitochondria in a subject. In some embodiments, the mitochondrial disease is associated with, or is a result of, or is caused by mitochondrial dysfunction, one or more mitochondrial protein mutations, or one or more mitochondrial DNA mutations. In some embodiments, the mitochondrial disease is a mitochondrial myopathy. In some embodiments, mitochondrial diseases, e.g., the mitochondrial myopathy, that may be treated with a compound of Formula (I) or a pharmaceutically acceptable salt, solvate, hydrate, tautomer, ester, N-oxide or stereoisomer thereof include, e.g., Barth syndrome, chronic progressive external ophthalmoplegia (cPEO), Kearns-Sayre syndrome (KSS), Leigh syndrome (e.g., MILS, or maternally inherited Leigh syndrome), mitochondrial DNA depletion syndromes (MDDS, e.g., Alpers syndrome), mitochondrial encephalomyopathy (e.g., mitochondrial encephalomyopathy, lactic acidosis, and stroke-like episodes (MELAS)), mitochondrial neurogastrointestinal encephalomyopathy (MNGIE), myoclonus epilepsy with ragged red fibers (MERRF), neuropathy, ataxia, retinitis pigmentosa (NARP), Leber's hereditary optic neuropathy (LHON), and Pearson syndrome.

In some embodiments, the compound of Formula (I) or a pharmaceutically acceptable salt, solvate, hydrate, tautomer, ester, N-oxide or stereoisomer thereof is used to treat a mitochondrial disease described herein by decreasing or eliminating a symptom of the disease. In some embodiments, the compound of Formula (I) or a pharmaceutically acceptable salt, solvate, hydrate, tautomer, ester, N-oxide or stereoisomer thereof may be used as a single agent in a composition or in combination with another agent in a composition to treat a mitochondrial disease described herein.

### Hearing Loss

In some embodiments, the compound of Formula (I) or a pharmaceutically acceptable salt, solvate, hydrate, tautomer, ester, N-oxide or stereoisomer thereof is used to treat hearing loss. As used herein, the term “hearing loss” or “hearing loss condition” may broadly encompass any damage to the auditory systems, organs, and cells or any impairment of an animal subject's ability to hear sound, as measured by standard methods and assessments known in the art, for example otoacoustic emission testing, pure tone testing, and auditory brainstem response testing. Exemplary hearing loss conditions that may be treated with a compound of Formula (I), or a pharmaceutically acceptable salt, solvate, hydrate, tautomer, ester, N-oxide or stereoisomer thereof include, but are not limited to, mitochondrial nonsyndromic hearing loss and deafness, hair cell death, age-related hearing loss, noise-induced hearing loss, genetic or inherited hearing loss, hearing loss experienced as a result of ototoxic exposure, hearing loss resulting from disease, and hearing loss resulting from trauma. In some embodiments, mitochondrial nonsyndromic hearing loss and deafness is a MT-RNR1-related hearing loss. In some embodiments, the MT-RNR1-related hearing loss is the result of amino glycoside ototoxicity. In some embodiments, mitochondrial nonsyndromic hearing loss and deafness is a MT-TS1-related hearing loss. In some embodiments, mitochondrial nonsyndromic hearing loss and deafness is characterized by sensorineural hearing loss.

In some embodiments, the compound of Formula (I) or a pharmaceutically acceptable salt, solvate, hydrate, tautomer, ester, N-oxide or stereoisomer thereof is used to treat a hearing loss condition described herein by decreasing or eliminating a symptom of the disease. In some embodiments, the compound of Formula (I) or a pharmaceutically acceptable salt, solvate, hydrate, tautomer, ester, N-oxide or stereoisomer thereof may be used as a single agent in a composition or in combination with another agent in a composition to treat a hearing loss condition described herein.

### Ocular Disease

In some embodiments, the compound of Formula (I) or a pharmaceutically acceptable salt, solvate, hydrate, tautomer, ester, N-oxide or stereoisomer thereof is used to treat eye disease. As used herein, the term “ocular disease” may refer to a disease or condition in which the function of a subject's eye becomes impaired. Exemplary ocular diseases and conditions that may be treated with a compound of Formula (I), or a pharmaceutically acceptable salt, solvate, hydrate, tautomer, ester, N-oxide or stereoisomer thereof include cataracts, glaucoma, endoplasmic reticulum (ER) stress, autophagy deficiency, age-related macular degeneration (AMD), or diabetic retinopathy.

In some embodiments, the compound of Formula (I) or a pharmaceutically acceptable salt, solvate, hydrate, tautomer, ester, N-oxide or stereoisomer thereof is used to treat an ocular disease or condition described herein by decreasing or eliminating a symptom of the disease. In some embodiments, the compound of Formula (I) or a pharmaceutically acceptable salt, solvate, hydrate, tautomer, ester, N-oxide or stereoisomer thereof may be used as a single agent in a composition or in combination with another agent in a composition to treat an ocular disease or condition described herein.

### Kidney Diseases

In some embodiments, the compound of Formula (I) or a pharmaceutically acceptable salt, solvate, hydrate, tautomer, ester, N-oxide or stereoisomer thereof is used to treat kidney disease. As used herein, the term “kidney disease” may refer to a disease or condition in which the function of a subject's kidneys becomes impaired. Exemplary kidney diseases that may be treated with a compound of Formula (I), or a pharmaceutically acceptable salt, solvate, hydrate, tautomer, ester, N-oxide or stereoisomer thereof include Abderhalden-Kaufmann-Lignac syndrome (Nephropathic Cystinosis), Abdominal Compartment Syndrome, Acetaminophen-induced Nephrotoxicity, Acute Kidney Failure/Acute Kidney Injury, Acute Lobar Nephronia, Acute Phosphate Nephropathy, Acute Tubular Necrosis, Adenine Phosphoribosyltransferase Deficiency, Adenovirus Nephritis, Alagille Syndrome, Alport Syndrome, Amyloidosis, ANCA Vasculitis Related to Endocarditis and Other Infections, Angiomyolipoma, Analgesic Nephropathy, Anorexia Nervosa and Kidney Disease, Angiotensin Antibodies and Focal Segmental Glomerulosclerosis, Antiphospholipid Syndrome, Anti-TNF-α Therapy-related Glomerulonephritis, APOL1 Mutations, Apparent Mineralocorticoid Excess Syndrome, Aristolochic Acid Nephropathy, Chinese Herbal Nephropathy, Balkan Endemic Nephropathy, Arteriovenous Malformations and Fistulas of the Urologic Tract, Autosomal Dominant Hypocalcemia, Bardet-Biedl Syndrome, Bartter Syndrome, Bath Salts and Acute Kidney Injury, Beer Potomania, Beeturia, β-Thalassemia Renal Disease, Bile Cast Nephropathy, BK Polyoma Virus Nephropathy in the Native Kidney, Bladder Rupture, Bladder Sphincter Dyssynergia, Bladder Tamponade, Border-Crossers' Nephropathy, Bourbon Virus and Acute Kidney Injury, Burnt Sugarcane Harvesting and Acute Renal Dysfunction, Byetta and Renal Failure, C1q Nephropathy, C3 Glomerulopathy, C3 Glomerulopathy with Monoclonal Gammopathy, C4 Glomerulopathy, Calcineurin Inhibitor Nephrotoxicity, Callilepsis Laureola Poisoning, Cannabinoid Hyperemesis Acute Renal Failure, Cardiorenal syndrome, Carfilzomib-Indiced Renal Injury, CFHR5 nephropathy, Charcot-Marie-Tooth Disease with Glomerulopathy, Chinese Herbal Medicines and Nephrotoxicity, Cherry Concentrate and Acute Kidney Injury, Cholesterol Emboli, Churg-Strauss syndrome, Chyluria, Ciliopathy, Cocaine and the Kidney, Cold Diuresis, Colistin Nephrotoxicity, Collagenofibrotic Glomerulopathy, Collapsing Glomerulopathy, Collapsing Glomerulopathy Related to CMV, Combination Antiretroviral (cART) Related-Nephropathy, Congenital Anomalies of the Kidney and Urinary Tract (CAKUT), Congenital Nephrotic Syndrome, Congestive Renal Failure, Conorenal syndrome (Mainzer-Saldino Syndrome or Saldino-Mainzer Disease), Contrast Nephropathy, Copper Sulphate Intoxication, Cortical Necrosis, Crizotinib-related Acute Kidney Injury, Cryocrystalglobulinemia, Cryoglobuinemia, Crystalglobulin-Induced Nephropathy, Crystal-Induced Acute Kidney injury, Crystal-Storing Histiocytosis, Cystic Kidney Disease, Acquired, Cystinuria, Dasatinib-Induced Nephrotic-Range Proteinuria, Dense Deposit Disease (MPGN Type 2), Dent Disease (X-linked Recessive Nephrolithiasis), DHA Crystalline Nephropathy, Dialysis Disequilibrium Syndrome, Diabetes and Diabetic Kidney Disease, Diabetes Insipidus, Dietary Supplements and Renal Failure, Diffuse Mesangial Sclerosis, Diuresis, Djenkol Bean Poisoning (Djenkolism), Down Syndrome and Kidney Disease, Drugs of Abuse and Kidney Disease, Duplicated Ureter, EAST syndrome, Ebola and the Kidney, Ectopic Kidney, Ectopic Ureter, Edema, Swelling, Erdheim-Chester Disease, Fabry's Disease, Familial Hypocalciuric Hypercalcemia, Fanconi Syndrome, Fraser syndrome, Fibronectin Glomerulopathy, Fibrillary Glomerulonephritis and Immunotactoid Glomerulopathy, Fraley syndrome, Fluid Overload, Hypervolemia, Focal Segmental Glomerulosclerosis, Focal Sclerosis, Focal Glomerulosclerosis, Galloway Mowat syndrome, Giant Cell (Temporal) Arteritis with Kidney Involvement, Gestational Hypertension, Gitelman Syndrome, Glomerular Diseases, Glomerular Tubular Reflux, Glycosuria, Goodpasture Syndrome, Green Smoothie Cleanse Nephropathy, HANAC Syndrome, Harvoni (Ledipasvir with Sofosbuvir)-Induced Renal Injury, Hair Dye Ingestion and Acute Kidney Injury, Hantavirus Infection Podocytopathy, Heat Stress Nephropathy, Hematuria (Blood in Urine), Hemolytic Uremic Syndrome (HUS), Atypical Hemolytic Uremic Syndrome (aHUS), Hemophagocytic Syndrome, Hemorrhagic Cystitis, Hemorrhagic Fever with Renal Syndrome (HFRS, Hantavirus Renal Disease, Korean Hemorrhagic Fever, Epidemic Hemorrhagic Fever, Nephropathis Epidemica), Hemosiderinuria, Hemosiderosis related to Paroxysmal Nocturnal Hemoglobinuria and Hemolytic Anemia, Hepatic Glomerulopathy, Hepatic Veno-Occlusive Disease, Sinusoidal Obstruction Syndrome, Hepatitis C-Associated Renal Disease, Hepatocyte Nuclear Factor 1P-Associated Kidney Disease, Hepatorenal Syndrome, Herbal Supplements and Kidney Disease, High Altitude Renal Syndrome, High Blood Pressure and Kidney Disease, HIV-Associated Immune Complex Kidney Disease (HIVICK), HIV-Associated Nephropathy (HIVAN), HNF1B-related Autosomal Dominant Tubulointerstitial Kidney Disease, Horseshoe Kidney (Renal Fusion), Hunner's Ulcer, Hydroxychloroquine-induced Renal Phospholipidosis, Hyperaldosteronism, Hypercalcemia, Hyperkalemia, Hypermagnesemia, Hypernatremia, Hyperoxaluria, Hyperphosphatemia, Hypocalcemia, Hypocomplementemic Urticarial Vasculitic Syndrome, Hypokalemia, Hypokalemia-induced renal dysfunction, Hypokalemic Periodic Paralysis, Hypomagnesemia, Hyponatremia, Hypophosphatemia, Hypophosphatemia in Users of Cannabis, Hypertension, Hypertension, Monogenic, Iced Tea Nephropathy, Ifosfamide Nephrotoxicity, IgA Nephropathy, IgG4 Nephropathy, Immersion Diuresis, Immune-Checkpoint Therapy-Related Interstitial Nephritis, Infliximab-Related Renal Disease, Interstitial Cystitis, Painful Bladder Syndrome (Questionnaire), Interstitial Nephritis, Interstitial Nephritis, Karyomegalic, Ivemark's syndrome, JC Virus Nephropathy, Joubert Syndrome, Ketamine-Associated Bladder Dysfunction, Kidney Stones, Nephrolithiasis, Kombucha Tea Toxicity, Lead Nephropathy and Lead-Related Nephrotoxicity, Lecithin Cholesterol Acyltransferase Deficiency (LCAT Deficiency), Leptospirosis Renal Disease, Light Chain Deposition Disease, Monoclonal Immunoglobulin Deposition Disease, Light Chain Proximal Tubulopathy, Liddle Syndrome, Lightwood-Albright Syndrome, Lipoprotein Glomerulopathy, Lithium Nephrotoxicity, LMX1B Mutations Cause Hereditary FSGS, Loin Pain Hematuria, Lupus, Systemic Lupus Erythematosis, Lupus Kidney Disease, Lupus Nephritis, Lupus Nephritis with Antineutrophil Cytoplasmic Antibody Seropositivity, Lupus Podocytopathy, Lyme Disease-Associated Glomerulonephritis, Lysinuric Protein Intolerance, Lysozyme Nephropathy, Malarial Nephropathy, Malignancy-Associated Renal Disease, Malignant Hypertension, Malakoplakia, McKittrick-Wheelock Syndrome, MDMA (Molly; Ecstacy; 3,4-Methylenedioxymethamphetamine) and Kidney Failure, Meatal Stenosis, Medullary Cystic Kidney Disease, Urolodulin-Associated Nephropathy, Juvenile Hyperuricemic Nephropathy Type 1, Medullary Sponge Kidney, Megaureter, Melamine Toxicity and the Kidney, MELAS Syndrome, Membranoproliferative Glomerulonephritis, Membranous Nephropathy, Membranous-like Glomerulopathy with Masked IgG Kappa Deposits, MesoAmerican Nephropathy, Metabolic Acidosis, Metabolic Alkalosis, Methotrexate-related Renal Failure, Microscopic Polyangiitis, Milk-alkalai syndrome, Minimal Change Disease, Monoclonal Gammopathy of Renal Significance, Dysproteinemia, Mouthwash Toxicity, MUC1 Nephropathy, Multicystic dysplastic kidney, Multiple Myeloma, Myeloproliferative Neoplasms and Glomerulopathy, Nail-patella Syndrome, NARP Syndrome, Nephrocalcinosis, Nephrogenic Systemic Fibrosis, Nephroptosis (Floating Kidney, Renal Ptosis), Nephrotic Syndrome, Neurogenic Bladder, 9/11 and Kidney Disease, Nodular Glomerulosclerosis, Non-Gonococcal Urethritis, Nutcracker syndrome, Oligomeganephronia, Orofaciodigital Syndrome, Orotic Aciduria, Orthostatic Hypotension, Orthostatic Proteinuria, Osmotic Diuresis, Osmotic Nephrosis, Ovarian Hyperstimulation Syndrome, Oxalate Nephropathy, Page Kidney, Papillary Necrosis, Papillorenal Syndrome (Renal-Coloboma Syndrome, Isolated Renal Hypoplasia), PARN Mutations and Kidney Disease, Parvovirus B19 and the Kidney, The Peritoneal-Renal Syndrome, POEMS Syndrome, Posterior Urethral Valve, Podocyte Infolding Glomerulopathy, Post-infectious Glomerulonephritis, Post-streptococcal Glomerulonephritis, Post-infectious Glomerulonephritis, Atypical, Post-Infectious Glomerulonephritis (IgA-Dominant), Mimicking IgA Nephropathy, Polyarteritis Nodosa, Polycystic Kidney Disease, Posterior Urethral Valves, Post-Obstructive Diuresis, Preeclampsia, Propofol infusion syndrome, Proliferative Glomerulonephritis with Monoclonal IgG Deposits (Nasr Disease), Propolis (Honeybee Resin) Related Renal Failure, Proteinuria (Protein in Urine), Pseudohyperaldosteronism, Pseudohypobicarbonatemia, Pseudohypoparathyroidism, Pulmonary-Renal Syndrome, Pyelonephritis (Kidney Infection), Pyonephrosis, Pyridium and Kidney Failure, Radiation Nephropathy, Ranolazine and the Kidney, Refeeding syndrome, Reflux Nephropathy, Rapidly Progressive Glomerulonephritis, Renal Abscess, Peripnephric Abscess, Renal Agenesis, Renal Arcuate Vein Microthrombi-Associated Acute Kidney Injury, Renal Artery Aneurysm, Renal Artery Dissection, Spontaneous, Renal Artery Stenosis, Renal Cell Cancer, Renal Cyst, Renal Hypouricemia with Exercise-induced Acute Renal Failure, Renal Infarction, Renal Osteodystrophy, Renal Tubular Acidosis, Renin Mutations and Autosomal Dominant Tubulointerstitial Kidney Disease, Renin Secreting Tumors (Juxtaglomerular Cell Tumor), Reset Osmostat, Retrocaval Ureter, Retroperitoneal Fibrosis, Rhabdomyolysis, Rhabdomyolysis related to Bariatric Sugery, Rheumatoid Arthritis-Associated Renal Disease, Sarcoidosis Renal Disease, Salt Wasting, Renal and Cerebral, Schistosomiasis and Glomerular Disease, Schimke immuno-osseous dysplasia, Scleroderma Renal Crisis, Serpentine Fibula-Polycystic Kidney Syndrome, Exner Syndrome, Sickle Cell Nephropathy, Silica Exposure and Chronic Kidney Disease, Sri Lankan Farmers' Kidney Disease, Sjogren's Syndrome and Renal Disease, Synthetic Cannabinoid Use and Acute Kidney Injury, Kidney Disease Following Hematopoietic Cell Transplantation, Kidney Disease Related to Stem Cell Transplantation, TAFRO Syndrome, Tea and Toast Hyponatremia, Tenofovir-Induced Nephrotoxicity, Thin Basement Membrane Disease, Benign Familial Hematuria, Thrombotic Microangiopathy Associated with Monoclonal Gammopathy, Trench Nephritis, Trigonitis, Tuberculosis, Genitourinary, Tuberous Sclerosis, Tubular Dysgenesis, Immune Complex Tubulointerstitial Nephritis Due to Autoantibodies to the Proximal Tubule Brush Border, Tumor Lysis Syndrome, Uremia, Uremic Optic Neuropathy, Ureteritis Cystica, Ureterocele, Urethral Caruncle, Urethral Stricture, Urinary Incontinence, Urinary Tract Infection, Urinary Tract Obstruction, Urogenital Fistula, Uromodulin-Associated Kidney Disease, Vancomycin-Associated Cast Nephropathy, Vasomotor Nephropathy, Vesicointestinal Fistula, Vesicoureteral Reflux, VGEF Inhibition and Renal Thrombotic Microangiopathy, Volatile Anesthetics and Acute Kidney Injury, Von Hippel-Lindau Disease, Waldenstrom's Macroglobulinemic Glomerulonephritis, Warfarin-Related Nephropathy, Wasp Stings and Acute Kidney Injury, Wegener's Granulomatosis, Granulomatosis with Polyangiitis, West Nile Virus and Chronic Kidney Disease, Wunderlich syndrome, Zellweger Syndrome, or Cerebrohepatorenal Syndrome.

In some embodiments, the compound of Formula (I) or a pharmaceutically acceptable salt, solvate, hydrate, tautomer, ester, N-oxide or stereoisomer thereof is used to treat a kidney disease described herein by decreasing or eliminating a symptom of the disease. In some embodiments, the compound of Formula (I) or a pharmaceutically acceptable salt, solvate, hydrate, tautomer, ester, N-oxide or stereoisomer thereof may be used as a single agent in a composition or in combination with another agent in a composition to treat a kidney disease described herein.

### Skin Diseases

In some embodiments, the compound of Formula (I) or a pharmaceutically acceptable salt, solvate, hydrate, tautomer, ester, N-oxide or stereoisomer thereof is used to treat a skin disease. As used herein, the term “skin disease” may refer to a disease or condition affecting the skin. Exemplary skin diseases that may be treated with a compound of Formula (I), or a pharmaceutically acceptable salt, solvate, hydrate, tautomer, ester, N-oxide or stereoisomer thereof include acne, alopecia areata, basal cell carcinoma, Bowen's disease, congenital erythropoietic porphyria, contact dermatitis, Darier's disease, disseminated superficial actinic porokeratosis, dystrophic epidermolysis bullosa, eczema (atopic eczema), extra-mammary Paget's disease, epidermolysis bullosa simplex, erythropoietic protoporphyria, fungal infections of nails, Hailey-Hailey disease, herpes simplex, hidradenitis suppurativa, hirsutism, hyperhidrosis, ichthyosis, impetigo, keloids, keratosis pilaris, lichen planus, lichen sclerosus, melanoma, melasma, mucous membrane pemphigoid, pemphigoid, Pemphigus vulgaris, Pityriasis lichenoides, Pityriasis rubra pilaris, plantar warts (verrucas), polymorphic light eruption, psoriasis, plaque psoriasis, pyoderma gangrenosum, rosacea, scabies, scleroderma, shingles, squamous cell carcinoma, sweet's syndrome, urticaria and angioedema and vitiligo.

In some embodiments, the compound of Formula (I) or a pharmaceutically acceptable salt, solvate, hydrate, tautomer, ester, N-oxide or stereoisomer thereof is used to treat a skin disease described herein by decreasing or eliminating a symptom of the disease. In some embodiments, the compound of Formula (I) or a pharmaceutically acceptable salt, solvate, hydrate, tautomer, ester, N-oxide or stereoisomer thereof may be used as a single agent in a composition or in combination with another agent in a composition to treat a skin disease described herein.

### Fibrotic Diseases

In some embodiments, the compound of Formula (I) or a pharmaceutically acceptable salt, solvate, hydrate, tautomer, ester, N-oxide or stereoisomer thereof is used to treat a fibrotic disease. As used herein, the term “fibrotic disease” may refer to a disease or condition that is defined by the accumulation of excess extracellular matrix components. Exemplary fibrotic diseases that may be treated with a compound of Formula (I), or a pharmaceutically acceptable salt, solvate, hydrate, tautomer, ester, N-oxide or stereoisomer thereof include adhesive capsulitis, arterial stiffness, arthrofibrosis, atrial fibrosis, cardiac fibrosis, cirrhosis, congenital hepatic fibrosis, Crohn's disease, cystic fibrosis, Dupuytren's contracture, endomyocardial fibrosis, glial scar, hepatitis C, hypertrophic cardiomyopathy, hypersensitivity pneumonitis, idiopathic pulmonary fibrosis, idiopathic interstitial pneumonia, interstitial lung disease, keloid, mediastinal fibrosis, myelofibrosis, nephrogenic systemic fibrosis, non-alcoholic fatty liver disease, old myocardial infarction, Peyronie's disease, pneumoconiosis, pneumonitis, progressive massive fibrosis, pulmonary fibrosis, radiation-induced lung injury, retroperitoneal fibrosis, scleroderma/systemic sclerosis, silicosis and ventricular remodeling.

In some embodiments, the compound of Formula (I) or a pharmaceutically acceptable salt, solvate, hydrate, tautomer, ester, N-oxide or stereoisomer thereof is used to treat a fibrotic disease described herein by decreasing or eliminating a symptom of the disease. In some embodiments, the compound of Formula (I) or a pharmaceutically acceptable salt, solvate, hydrate, tautomer, ester, N-oxide or stereoisomer thereof may be used as a single agent in a composition or in combination with another agent in a composition to treat a fibrotic disease described herein.

### Hemoglobin Disorders

In some embodiments, the compound of Formula (I) or a pharmaceutically acceptable salt, solvate, hydrate, tautomer, ester, N-oxide or stereoisomer thereof is used to treat a hemoglobin disease. As used herein, the terms “hemoglobin disease” or “hemoglobin disorder” may refer to a disease or condition characterized by an abnormal production or structure of the hemoglobin protein. Exemplary hemoglobin diseases that may be treated with a compound of Formula (I), or a pharmaceutically acceptable salt, solvate, hydrate, tautomer, ester, N-oxide or stereoisomer thereof include “dominant” β-thalassemia, acquired (toxic) methemoglobinemia, carboxyhemoglobinemia, congenital Heinz body hemolytic anemia, HbH disease, HbS/β-thalassemia, HbE/β-thalassemia, HbSC disease, homozygous a-thalassemia (phenotype of α⁰-thalassemia), Hydrops fetalis with Hb Bart's, sickle cell anemia/disease, sickle cell trait, sickle β-thalassemia disease, α⁺-thalassemia, α⁰-thalassemia, α-Thalassemia associated with myelodysplastic syndromes, α-Thalassemia with mental retardation syndrome (ATR), β⁰-Thalassemia, β⁺-Thalassemia, δ-Thalassemia, γ-Thalassemia, β-Thalassemia major, β-Thalassemia intermedia, δβ-Thalassemia, and εγδβ-Thalassemia.

In some embodiments, the compound of Formula (I) or a pharmaceutically acceptable salt, solvate, hydrate, tautomer, ester, N-oxide or stereoisomer thereof is used to treat a hemoglobin disease described herein by decreasing or eliminating a symptom of the disease. In some embodiments, the compound of Formula (I) or a pharmaceutically acceptable salt, solvate, hydrate, tautomer, ester, N-oxide or stereoisomer thereof may be used as a single agent in a composition or in combination with another agent in a composition to treat a hemoglobin disease described herein.

### Autoimmune Diseases

In some embodiments, the compound of Formula (I) or a pharmaceutically acceptable salt, solvate, hydrate, tautomer, ester, N-oxide or stereoisomer thereof is used to treat an autoimmune disease. As used herein, the term “autoimmune disease” may refer to a disease or condition in which the immune system of a subject attacks and damages the tissues of said subject. Exemplary kidney diseases that may be treated with a compound of Formula (I), or a pharmaceutically acceptable salt, solvate, hydrate, tautomer, ester, N-oxide or stereoisomer thereof include Achalasia, Addison's disease, Adult Still's disease, Agammaglobulinemia, Alopecia areata, Amyloidosis, Ankylosing spondylitis, Anti-GBM/Anti-TBM nephritis, Antiphospholipid syndrome, Autoimmune angioedema, Autoimmune dysautonomia, Autoimmune encephalomyelitis, Autoimmune hepatitis, Autoimmune inner ear disease (AIED), Autoimmune myocarditis, Autoimmune oophoritis, Autoimmune orchitis, Autoimmune pancreatitis, Autoimmune retinopathy, Autoimmune urticaria, Axonal & neuronal neuropathy (AMAN), Baló disease, Behcet's disease, Benign mucosal pemphigoid, Bullous pemphigoid, Castleman disease (CD), Celiac disease, Chagas disease, Chronic inflammatory demyelinating polyneuropathy (CIDP), Chronic recurrent multifocal osteomyelitis (CRMO), Churg-Strauss Syndrome (CSS) or Eosinophilic Granulomatosis (EGPA), Cicatricial pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease (neuromyelitis optica), Discoid lupus, Dressler's syndrome, Endometriosis, Eosinophilic esophagitis (EoE), Eosinophilic fasciitis, Erythema nodosum, Essential mixed cryoglobulinemia, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's thyroiditis, Hemolytic anemia, Henoch-Schonlein purpura (HSP), Herpes gestationis or pemphigoid gestationis (PG), Hidradenitis Suppurativa (HS) (Acne Inversa), Hypogammalglobulinemia, IgA Nephropathy, IgG4-related sclerosing disease, Immune thrombocytopenic purpura (ITP), Inclusion body myositis (IBM), Interstitial cystitis (IC), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus, Lyme disease chronic, Meniere's disease, Microscopic polyangiitis (MPA), Mixed connective tissue disease (MCTD), Mooren's ulcer, Mucha-Habermann disease, Multifocal Motor Neuropathy (MMN) or MMNCB, Multiple sclerosis, Myasthenia gravis, Myositis, Narcolepsy, Neonatal Lupus, Neuromyelitis optica, Neutropenia, Ocular cicatricial pemphigoid, Optic neuritis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonnage-Turner syndrome, Pemphigus, Peripheral neuropathy, Perivenous encephalomyelitis, Pernicious anemia (PA), POEMS syndrome, Polyarteritis nodosa, Polyglandular syndrome type I, Polyglandular syndrome type II, Polyglandular syndrome type III, Polymyalgia rheumatica, Polymyositis, Postmyocardial infarction syndrome, Postpericardiotomy syndrome, Primary biliary cirrhosis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Sarcoidosis, Schmidt syndrome, Scleritis, Scleroderma, Sjogren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopenic purpura (TTP), Tolosa-Hunt syndrome (THS), Transverse myelitis, Type 1 diabetes, Ulcerative colitis (UC), Undifferentiated connective tissue disease (UCTD), Uveitis, Vasculitis, Vitiligo, Vogt-Koyanagi-Harada Disease, and Wegener's granulomatosis (or Granulomatosis with Polyangiitis (GPA)).

In some embodiments, the compound of Formula (I) or a pharmaceutically acceptable salt, solvate, hydrate, tautomer, ester, N-oxide or stereoisomer thereof is used to treat an autoimmune disease described herein by decreasing or eliminating a symptom of the disease. In some embodiments, the compound of Formula (I) or a pharmaceutically acceptable salt, solvate, hydrate, tautomer, ester, N-oxide or stereoisomer thereof may be used as a single agent in a composition or in combination with another agent in a composition to treat an autoimmune disease described herein.

### Viral Infections

In some embodiments, the compound of Formula (I) or a pharmaceutically acceptable salt, solvate, hydrate, tautomer, ester, N-oxide or stereoisomer thereof is used to treat a viral infection. Exemplary viral infections that may be treated with a compound of Formula (I), or a pharmaceutically acceptable salt, solvate, hydrate, tautomer, ester, N-oxide or stereoisomer thereof include influenza, human immunodeficiency virus (HIV) and herpes.

In some embodiments, the compound of Formula (I) or a pharmaceutically acceptable salt, solvate, hydrate, tautomer, ester, N-oxide or stereoisomer thereof is used to treat a viral infection described herein by decreasing or eliminating a symptom of the disease. In some embodiments, the compound of Formula (I) or a pharmaceutically acceptable salt, solvate, hydrate, tautomer, ester, N-oxide or stereoisomer thereof may be used as a single agent in a composition or in combination with another agent in a composition to treat a viral infection described herein.

### Malaria Infection

In some embodiments, the compound of Formula (I) or a pharmaceutically acceptable salt, solvate, hydrate, tautomer, ester, N-oxide or stereoisomer thereof is used to treat a malaria. As used herein, the term “malaria” may refer to a parasitic disease of protozoan of the plasmodium genus that causes infection of red blood cells (RBCs). Exemplary forms of malaria infection that may be treated with a compound of Formula (I), or a pharmaceutically acceptable salt, solvate, hydrate, tautomer, ester, N-oxide or stereoisomer thereof include infection caused by Plasmodium vivax, Plasmodium ovale, Plasmodium malariae and Plasmodium falciparum. In some embodiments, the malaria infection that may be treated with a compound of Formula (I), or a pharmaceutically acceptable salt, solvate, hydrate, tautomer, ester, N-oxide or stereoisomer thereof is resistant/recrudescent malaria.

In some embodiments, the compound of Formula (I) or a pharmaceutically acceptable salt, solvate, hydrate, tautomer, ester, N-oxide or stereoisomer thereof is used to treat a malaria infection described herein by decreasing or eliminating a symptom of the disease. In some embodiments, the compound of Formula (I) or a pharmaceutically acceptable salt, solvate, hydrate, tautomer, ester, N-oxide or stereoisomer thereof may be used as a single agent in a composition or in combination with another agent in a composition to treat a malaria infection described herein.

Diseases with Mutations Leading to Unfolded Protein Response (UPR) Induction

In some embodiments, the compound of Formula (I) or a pharmaceutically acceptable salt, solvate, hydrate, tautomer, ester, N-oxide or stereoisomer thereof is used to treat a disease with mutations that leads to UPR induction. Exemplary disease with mutations that lead to UPR induction include Marinesco-Sjogren syndrome, neuropathic pain, diabetic neuropathic pain, noise induced hearing loss, non-syndromic sensorineural hearing loss, age-related hearing loss, Wolfram syndrome, Darier White disease, Usher syndrome, collagenopathies, Thin basement nephropathy, Alport syndrome, skeletal chondrodysplasia, metaphyseal chondrodysplasia type Schmid, and Pseudochondrodysplasia.

In some embodiments, the compound of Formula (I) or a pharmaceutically acceptable salt, solvate, hydrate, tautomer, ester, N-oxide or stereoisomer thereof is used to treat a disease with mutations that leads to UPR induction described herein by decreasing or eliminating a symptom of the disease. In some embodiments, the compound of Formula (I) or a pharmaceutically acceptable salt, solvate, hydrate, tautomer, ester, N-oxide or stereoisomer thereof may be used as a single agent in a composition or in combination with another agent in a composition to treat a disease with mutations that leads to UPR induction described herein.

### Methods of Modulating Protein Production

In another aspect, disclosed herein is a method of modulating the expression of eIF2B, eIF2α, a component of the eIF2 pathway, component of the ISR pathway or any combination thereof in a cell, the method comprising contacting the cell with an effective amount of a compound of Formula (I), or a pharmaceutically acceptable salt, solvate, hydrate, tautomer, ester, N-oxide or stereoisomer thereof, thereby modulating the expression of eIF2B, eIF2α, a component of the eIF2 pathway, component of the ISR pathway or any combination thereof in the cell. In some embodiments, contacting the compound of Formula (I), or a pharmaceutically acceptable salt, solvate, hydrate, tautomer, ester, N-oxide or stereoisomer thereof with the cell increases the expression of eIF2B, eIF2α, a component of the eIF2 pathway, component of the ISR pathway or any combination thereof in the cell. In some embodiments, contacting the compound of Formula (I), or a pharmaceutically acceptable salt, solvate, hydrate, tautomer, ester, N-oxide or stereoisomer thereof with the cell decreases the expression of eIF2B, eIF2α, a component of the eIF2 pathway, component of the ISR pathway or any combination thereof in the cell.

In another aspect, disclosed herein is a method of preventing or treating a condition, disease or disorder described herein in a patient in need thereof, the method comprising administering to the patient an effective amount of a compound of Formula (I), or a pharmaceutically acceptable salt, solvate, hydrate, tautomer, ester, N-oxide or stereoisomer thereof, wherein the compound of Formula (I), or a pharmaceutically acceptable salt, solvate, hydrate, tautomer, ester, N-oxide or stereoisomer thereof modulates the expression of eIF2B, eIF2α, a component of the eIF2 pathway, component of the ISR pathway or any combination thereof by the patient's cells, thereby treating the condition, disease or disorder. In some embodiments, the condition, disease or disorder is characterized by aberrant expression of eIF2B, eIF2α, a component of the eIF2 pathway, component of the ISR pathway or any combination thereof by the patient's cells. In some embodiments, the compound of Formula (I), or a pharmaceutically acceptable salt, solvate, hydrate, tautomer, ester, N-oxide or stereoisomer thereof increases the expression of eIF2B, eIF2α, a component of the eIF2 pathway, component of the ISR pathway or any combination thereof by the patient's cells, thereby treating the condition, disease or disorder. In some embodiments, the compound of Formula (I), or a pharmaceutically acceptable salt, solvate, hydrate, tautomer, ester, N-oxide or stereoisomer thereof decreases the expression of eIF2B, eIF2α, a component of the eIF2 pathway, component of the ISR pathway or any combination thereof by the patient's cells, thereby treating the condition, disease or disorder.

In another aspect, disclosed herein is a method of modulating the activity of eIF2B, eIF2α, a component of the eIF2 pathway, component of the ISR pathway or any combination thereof in a cell, the method comprising contacting the cell with an effective amount of a compound of Formula (I), or a pharmaceutically acceptable salt, solvate, hydrate, tautomer, ester, N-oxide or stereoisomer thereof, thereby modulating the activity of eIF2B, eIF2α, a component of the eIF2 pathway, component of the ISR pathway or any combination thereof in the cell. In some embodiments, contacting the compound of Formula (I), or a pharmaceutically acceptable salt, solvate, hydrate, tautomer, ester, N-oxide or stereoisomer thereof with the cell increases the activity of eIF2B, eIF2α, a component of the eIF2 pathway, component of the ISR pathway or any combination thereof in the cell. In some embodiments, contacting the compound of Formula (I), or a pharmaceutically acceptable salt, solvate, hydrate, tautomer, ester, N-oxide or stereoisomer thereof with the cell decreases the activity of eIF2B, eIF2α, a component of the eIF2 pathway, component of the ISR pathway or any combination thereof in the cell.

In another aspect, disclosed herein is a method of preventing or treating a condition, disease or disorder described herein in a patient in need thereof, the method comprising administering to the patient an effective amount of a compound of Formula (I), or a pharmaceutically acceptable salt, solvate, hydrate, tautomer, ester, N-oxide or stereoisomer thereof, wherein the compound of Formula (I), or a pharmaceutically acceptable salt, solvate, hydrate, tautomer, ester, N-oxide or stereoisomer thereof modulates the activity of eIF2B, eIF2α, a component of the eIF2 pathway, component of the ISR pathway or any combination thereof by the patients cells, thereby treating the condition, disease or disorder. In some embodiments, the condition, disease or disorder is characterized by aberrant activity of eIF2B, eIF2α, a component of the eIF2 pathway, component of the ISR pathway or any combination thereof in the patient's cells. In some embodiments, the compound of Formula (I), or a pharmaceutically acceptable salt, solvate, hydrate, tautomer, ester, N-oxide or stereoisomer thereof increases the activity of eIF2B, eIF2α, a component of the eIF2 pathway, component of the ISR pathway or any combination thereof in the patient's cells, thereby treating the condition, disease or disorder. In some embodiments, the compound of Formula (I), or a pharmaceutically acceptable salt, solvate, hydrate, tautomer, ester, N-oxide or stereoisomer thereof decreases the activity of eIF2B, eIF2α, a component of the eIF2 pathway, component of the ISR pathway or any combination thereof in the patient's cells, thereby treating the condition, disease or disorder.

In some embodiments, administering an effective amount of a compound of Formula (I), or a pharmaceutically acceptable salt, solvate, hydrate, tautomer, ester, N-oxide or stereoisomer thereof, wherein the compound of Formula (I), or a pharmaceutically acceptable salt, solvate, hydrate, tautomer, ester, N-oxide or stereoisomer thereof modulates both the expression and the activity of eIF2B, eIF2α, a component of the eIF2 pathway, component of the ISR pathway or any combination thereof in the patients cells, thereby treating the condition, disease or disorder.

In some embodiments, the compound of Formula (I) is chemically modified, prior to (ex vivo) or after (in vivo) contacting with a cell, forming a biologically active compound that modulates the expression and/or activity of eIF2B, eIF2α, a component of the eIF2 pathway, component of the ISR pathway or any combination thereof in the cell. In some embodiments, the compound of Formula (I) is metabolized by the patient forming a biologically active compound that modulates the expression and/or activity of eIF2B, eIF2α, a component of the eIF2 pathway, component of the ISR pathway or any combination thereof in the patients cells, thereby treating a condition, disease or disorder disclosed herein. In some embodiments, the biologically active compound is the compound of formula (II).

In one aspect, disclosed herein is a method of treating a disease related to a modulation of eIF2B activity or levels, eIF2α activity or levels, or the activity or levels of a component of the eIF2 pathway or the ISR pathway in a patient in need thereof, comprising administering to the patient an effective amount of a compound of Formula (I). In some embodiments, the modulation comprises an increase in eIF2B activity or levels, increase in eIF2α activity or levels, or increase in activity or levels of a component of the eIF2 pathway or the ISR pathway. In some embodiments, the disease may be caused by a mutation to a gene or protein sequence related to a member of the eIF2 pathway (e.g., the eIF2α signaling pathway).

### Methods of Increasing Protein Activity and Production

In another aspect, the compound of Formula (I), or a pharmaceutically acceptable salt, solvate, hydrate, tautomer, ester, N-oxide or stereoisomer thereof may be useful in applications where increasing production output of eIF2B, eIF2α, a component of the eIF2 pathway, a component of the ISR pathway or any combination thereof is desirable, such as in vitro cell free systems for protein production.

In some embodiments, the present invention features a method of increasing expression of eIF2B, eIF2α, a component of the eIF2 pathway, a component of the ISR pathway or any combination thereof by a cell or in vitro expression system, the method comprising contacting the cell or in vitro expression system with an effective amount of a compound of Formula (I), or a pharmaceutically acceptable salt, solvate, hydrate, tautomer, ester, N-oxide or stereoisomer thereof. In some embodiments, the method is a method of increasing the expression of eIF2B, eIF2α, a component of the eIF2 pathway, a component of the ISR pathway or any combination thereof by a cell comprising contacting the cell with an effective amount of a compound described herein (e.g., the compound of Formula (I), or a pharmaceutically acceptable salt, solvate, hydrate, tautomer, ester, N-oxide or stereoisomer thereof). In other embodiments, the method is a method of increasing the expression of eIF2B, eIF2α, a component of the eIF2 pathway, a component of the ISR pathway or any combination thereof by an in vitro protein expression system comprising contacting the in vitro expression system with a compound described herein (e.g. the compound of Formula (I), or a pharmaceutically acceptable salt, solvate, hydrate, tautomer, ester, N-oxide or stereoisomer thereof). In some embodiments, contacting the cell or in vitro expression system with an effective amount of a compound of Formula (I), or a pharmaceutically acceptable salt, solvate, hydrate, tautomer, ester, N-oxide or stereoisomer thereof increases expression of eIF2B, eIF2α, a component of the eIF2 pathway, a component of the ISR pathway or any combination thereof in the cell or in vitro expression system by about 1%, about 2%, about 3%, about 4%, about 5%, about 6%, about 7%, about 8%, about 9%, about 10%, about 15%, about 20%, about 25%, about 30%, about 40%, about 45%, about 50%, about 60%, about 65%, about 70%, about 75%, about 80%, about 85%, about 90%, about 95%, or about 100%. In some embodiments, contacting the cell or in vitro expression system with an effective amount of a compound of Formula (I), or a pharmaceutically acceptable salt, solvate, hydrate, tautomer, ester, N-oxide or stereoisomer thereof increases expression of eIF2B, eIF2α, a component of the eIF2 pathway, a component of the ISR pathway or any combination thereof in the cell or in vitro expression system by about 1-fold, about 2-fold, about 3-fold, about 4-fold, about 5-fold, about 6-fold, about 7-fold, about 8-fold, about 9-fold, about 10-fold, about 20-fold, about 30-fold, about 40-fold, about 50-fold, about 60-fold, about 70-fold, about 80-fold, about 90-fold, about 100-fold, about 200-fold, about 300-fold, about 400-fold, about 500-fold, about 600-fold about 700-fold, about 800-fold, about 900-fold, about 1000-fold, about 10000-fold, about 100000-fold, or about 1000000-fold.

In some embodiments, the present invention features a method of increasing the expression of eIF2B, eIF2α, a component of the eIF2 pathway, a component of the ISR pathway or any combination thereof by a patient cells, the method comprising administering to the patient an effective amount of a compound of Formula (I), or a pharmaceutically acceptable salt, solvate, hydrate, tautomer, ester, N-oxide or stereoisomer thereof, wherein the patient has been diagnosed with a disease, disorder, or condition disclosed herein and wherein the disease, disorder or condition is characterized by aberrant expression of eIF2B, eIF2α, a component of the eIF2 pathway, a component of the ISR pathway or any combination thereof (e.g., a leukodystrophy, a leukoencephalopathy, a hypomyelinating or demyelinating disease, muscle-wasting disease, or sarcopenia). In some embodiments, administering to the patient in need thereof an effective amount of a compound of Formula (I), or a pharmaceutically acceptable salt, solvate, hydrate, tautomer, ester, N-oxide or stereoisomer thereof increases the expression of eIF2B, eIF2α, a component of the eIF2 pathway, a component of the ISR pathway or any combination thereof by the patients cells about 1%, about 2%, about 3%, about 4%, about 5%, about 6%, about 7%, about 8%, about 9%, about 10%, about 15%, about 20%, about 25%, about 30%, about 40%, about 45%, about 50%, about 60%, about 65%, about 70%, about 75%, about 80%, about 85%, about 90%, about 95%, or about 100%, thereby treating the disease, disorder or condition. In some embodiments, administering to the patient in need thereof an effective amount of a compound of Formula (I), or a pharmaceutically acceptable salt, solvate, hydrate, tautomer, ester, N-oxide or stereoisomer thereof increases expression of eIF2B, eIF2α, a component of the eIF2 pathway, a component of the ISR pathway or any combination thereof by the patients cells about 1-fold, about 2-fold, about 3-fold, about 4-fold, about 5-fold, about 6-fold, about 7-fold, about 8-fold, about 9-fold, about 10-fold, about 20-fold, about 30-fold, about 40-fold, about 50-fold, about 60-fold, about 70-fold, about 80-fold, about 90-fold, about 100-fold, about 200-fold, about 300-fold, about 400-fold, about 500-fold, about 600-fold about 700-fold, about 800-fold, about 900-fold, about 1000-fold, about 10000-fold, about 100000-fold, or about 1000000-fold, thereby treating the disease, disorder or condition.

In another aspect, the compound of Formula (I), or a pharmaceutically acceptable salt, solvate, hydrate, tautomer, ester, N-oxide or stereoisomer thereof may be useful in applications where increasing the activity of eIF2B, eIF2α, a component of the eIF2 pathway, a component of the ISR pathway or any combination thereof is desirable.

In some embodiments, the present invention features a method of increasing the activity of eIF2B, eIF2α, a component of the eIF2 pathway, a component of the ISR pathway or any combination thereof in a cell, the method comprising contacting the cell with an effective amount of a compound of Formula (I), or a pharmaceutically acceptable salt, solvate, hydrate, tautomer, ester, N-oxide or stereoisomer thereof. In some embodiments, contacting the cell with an effective amount of a compound of Formula (I), or a pharmaceutically acceptable salt, solvate, hydrate, tautomer, ester, N-oxide or stereoisomer thereof increases the activity of eIF2B, eIF2α, a component of the eIF2 pathway, a component of the ISR pathway or any combination thereof in the cell by about 1%, about 2%, about 3%, about 4%, about 5%, about 6%, about 7%, about 8%, about 9%, about 10%, about 15%, about 20%, about 25%, about 30%, about 40%, about 45%, about 50%, about 60%, about 65%, about 70%, about 75%, about 80%, about 85%, about 90%, about 95%, or about 100%. In some embodiments, contacting the cell with an effective amount of a compound of Formula (I), or a pharmaceutically acceptable salt, solvate, hydrate, tautomer, ester, N-oxide or stereoisomer thereof increases the activity of eIF2B, eIF2α, a component of the eIF2 pathway, a component of the ISR pathway or any combination thereof in the cell by about 1-fold, about 2-fold, about 3-fold, about 4-fold, about 5-fold, about 6-fold, about 7-fold, about 8-fold, about 9-fold, about 10-fold, about 20-fold, about 30-fold, about 40-fold, about 50-fold, about 60-fold, about 70-fold, about 80-fold, about 90-fold, about 100-fold, about 200-fold, about 300-fold, about 400-fold, about 500-fold, about 600-fold about 700-fold, about 800-fold, about 900-fold, about 1000-fold, about 10000-fold, about 100000-fold, or about 1000000-fold.

In some embodiments, the present invention features a method of increasing the activity of eIF2B, eIF2α, a component of the eIF2 pathway, a component of the ISR pathway or any combination thereof in a patient in need thereof, the method comprising administering to the patient an effective amount of a compound of Formula (I), or a pharmaceutically acceptable salt, solvate, hydrate, tautomer, ester, N-oxide or stereoisomer thereof, wherein the patient has been diagnosed with a disease, disorder, or condition disclosed herein and wherein the disease, disorder or condition is characterized by lowered levels of protein activity. In some embodiments, administering to the patient in need thereof an effective amount of a compound of Formula (I), or a pharmaceutically acceptable salt, solvate, hydrate, tautomer, ester, N-oxide or stereoisomer thereof increases the activity of eIF2B, eIF2α, a component of the eIF2 pathway, a component of the ISR pathway or any combination thereof in the patient by about 1%, about 2%, about 3%, about 4%, about 5%, about 6%, about 7%, about 8%, about 9%, about 10%, about 15%, about 20%, about 25%, about 30%, about 40%, about 45%, about 50%, about 60%, about 65%, about 70%, about 75%, about 80%, about 85%, about 90%, about 95%, or about 100%, thereby treating the disease, disorder or condition. In some embodiments, administering to the patient in need thereof an effective amount of a compound of Formula (I), or a pharmaceutically acceptable salt, solvate, hydrate, tautomer, ester, N-oxide or stereoisomer thereof increases the activity of eIF2B, eIF2α, a component of the eIF2 pathway, a component of the ISR pathway or any combination thereof in the patient by about 1-fold, about 2-fold, about 3-fold, about 4-fold, about 5-fold, about 6-fold, about 7-fold, about 8-fold, about 9-fold, about 10-fold, about 20-fold, about 30-fold, about 40-fold, about 50-fold, about 60-fold, about 70-fold, about 80-fold, about 90-fold, about 100-fold, about 200-fold, about 300-fold, about 400-fold, about 500-fold, about 600-fold about 700-fold, about 800-fold, about 900-fold, about 1000-fold, about 10000-fold, about 100000-fold, or about 1000000-fold, thereby treating the disease, disorder or condition.

In some embodiments, the compound of Formula (I) is chemically modified, prior to (ex vivo) or after (in vivo) contacting with the cell or in vitro expression system, forming a biologically active compound that increases the expression and/or activity of eIF2B, eIF2α, a component of the eIF2 pathway, component of the ISR pathway or any combination thereof in the cells and/or in vitro expression system. In some embodiments, the compound of Formula (I) is metabolized by the patient forming a biologically active compound that increases the expression and/or activity of eIF2B, eIF2α, a component of the eIF2 pathway, component of the ISR pathway or any combination thereof in the patients cells, thereby treating a condition, disease or disorder disclosed herein. In some embodiments, the biologically active compound is the compound of formula (II).

### Methods of Decreasing Protein Activity and Production

In another aspect, the compound of Formula (I), or a pharmaceutically acceptable salt, solvate, hydrate, tautomer, ester, N-oxide or stereoisomer thereof may be useful in applications where decreasing production output of eIF2B, eIF2α, a component of the eIF2 pathway, a component of the ISR pathway or any combination thereof is desirable.

In some embodiments, the present invention features a method of decreasing expression of eIF2B, eIF2α, a component of the eIF2 pathway, a component of the ISR pathway or any combination thereof in a cell, the method comprising contacting the cells with an effective amount of a compound of Formula (I), or a pharmaceutically acceptable salt, solvate, hydrate, tautomer, ester, N-oxide or stereoisomer thereof. In some embodiments, contacting the cells with an effective amount of a compound of Formula (I), or a pharmaceutically acceptable salt, solvate, hydrate, tautomer, ester, N-oxide or stereoisomer thereof decreases expression of eIF2B, eIF2α, a component of the eIF2 pathway, a component of the ISR pathway or any combination thereof in the cell by about 1%, about 2%, about 3%, about 4%, about 5%, about 6%, about 7%, about 8%, about 9%, about 10%, about 15%, about 20%, about 25%, about 30%, about 40%, about 45%, about 50%, about 60%, about 65%, about 70%, about 75%, about 80%, about 85%, about 90%, about 95%, or about 100%.

In some embodiments, the present invention features a method of decreasing the expression of eIF2B, eIF2α, a component of the eIF2 pathway, a component of the ISR pathway or any combination thereof in a patient in need thereof, the method comprising administering to the patient an effective amount of a compound of Formula (I), or a pharmaceutically acceptable salt, solvate, hydrate, tautomer, ester, N-oxide or stereoisomer thereof, wherein the patient has been diagnosed with a disease, disorder, or condition described herein and wherein the disease, disorder or condition is characterized by increased levels of protein production. In some embodiments, administering to the patient in need thereof an effective amount of a compound of Formula (I), or a pharmaceutically acceptable salt, solvate, hydrate, tautomer, ester, N-oxide or stereoisomer thereof decreases the expression of eIF2B, eIF2α, a component of the eIF2 pathway, a component of the ISR pathway or any combination thereof in the patient by about 1%, about 2%, about 3%, about 4%, about 5%, about 6%, about 7%, about 8%, about 9%, about 10%, about 15%, about 20%, about 25%, about 30%, about 40%, about 45%, about 50%, about 60%, about 65%, about 70%, about 75%, about 80%, about 85%, about 90%, about 95%, or about 100%, thereby treating the disease, disorder or condition.

In another aspect, the compound of Formula (I), or a pharmaceutically acceptable salt, solvate, hydrate, tautomer, ester, N-oxide or stereoisomer thereof may be useful in applications where decreasing the activity of eIF2B, eIF2α, a component of the eIF2 pathway, a component of the ISR pathway or any combination thereof is desirable.

In some embodiments, the present invention features a method of decreasing the activity of eIF2B, eIF2α, a component of the eIF2 pathway, a component of the ISR pathway or any combination thereof in a cell, the method comprising contacting the cell with an effective amount of a compound of Formula (I), or a pharmaceutically acceptable salt, solvate, hydrate, tautomer, ester, N-oxide or stereoisomer thereof. In some embodiments, contacting the cell with an effective amount of a compound of Formula (I), or a pharmaceutically acceptable salt, solvate, hydrate, tautomer, ester, N-oxide or stereoisomer thereof decreases the activity of eIF2B, eIF2α, a component of the eIF2 pathway, a component of the ISR pathway or any combination thereof in the cell by about 1%, about 2%, about 3%, about 4%, about 5%, about 6%, about 7%, about 8%, about 9%, about 10%, about 15%, about 20%, about 25%, about 30%, about 40%, about 45%, about 50%, about 60%, about 65%, about 70%, about 75%, about 80%, about 85%, about 90%, about 95%, or about 100%, thereby treating the disease, disorder or condition.

In some embodiments, the present invention features a method of decreasing the activity of eIF2B, eIF2α, a component of the eIF2 pathway, a component of the ISR pathway or any combination thereof in a patient in need thereof, the method comprising administering to the patient an effective amount of a compound of Formula (I), or a pharmaceutically acceptable salt, solvate, hydrate, tautomer, ester, N-oxide or stereoisomer thereof, wherein the patient has been diagnosed with a disease, disorder, or condition described herein and wherein the disease, disorder or condition is characterized by increased levels of protein activity. In some embodiments, administering to the patient in need thereof an effective amount of a compound of Formula (I), or a pharmaceutically acceptable salt, solvate, hydrate, tautomer, ester, N-oxide or stereoisomer thereof decreases the activity of eIF2B, eIF2α, a component of the eIF2 pathway, a component of the ISR pathway or any combination thereof in the patient by about 1%, about 2%, about 3%, about 4%, about 5%, about 6%, about 7%, about 8%, about 9%, about 10%, about 15%, about 20%, about 25%, about 30%, about 40%, about 45%, about 50%, about 60%, about 65%, about 70%, about 75%, about 80%, about 85%, about 90%, about 95%, or about 100%, thereby treating the disease, disorder or condition.

In some embodiments, the compound of Formula (I) is chemically modified, prior to (ex vivo) or after (in vivo) contacting with a cell, forming a biologically active compound that decreases the expression and/or activity of eIF2B, eIF2α, a component of the eIF2 pathway, component of the ISR pathway or any combination thereof in the cell. In some embodiments, the compound of Formula (I) is metabolized by the patient forming a biologically active compound that decreases the expression and/or activity of eIF2B, eIF2α, a component of the eIF2 pathway, component of the ISR pathway or any combination thereof in the patients cells, thereby treating a condition, disease or disorder disclosed herein. In some embodiments, the biologically active compound is the compound of formula (I).

In some embodiments, the compounds set forth herein are provided as pharmaceutical compositions including a compound of Formula (I) or Formula (II) or a pharmaceutically acceptable salt, solvate, hydrate, tautomer, ester, N-oxide or stereoisomer thereof and a pharmaceutically acceptable excipient. In embodiments of the method, a compound of Formula (I) or Formula (II) or a pharmaceutically acceptable salt, solvate, hydrate, tautomer, ester, N-oxide or stereoisomer thereof, is co-administered with a second agent (e.g. therapeutic agent). In other embodiments of the method, a compound of Formula (I) or Formula (II) or a pharmaceutically acceptable salt, solvate, hydrate, tautomer, ester, N-oxide or stereoisomer thereof, is co-administered with a second agent (e.g. therapeutic agent), which is administered in a therapeutically effective amount. In embodiments, the second agent is an agent for improving memory.

### Combination Therapy

In one aspect, the present invention features a pharmaceutical composition comprising a compound of Formula (I) or a pharmaceutically acceptable salt, solvate, hydrate, tautomer, ester, N-oxide or stereoisomer thereof as well as a second agent (e.g. a second therapeutic agent). In some embodiments, the pharmaceutical composition includes a second agent (e.g. a second therapeutic agent) in a therapeutically effective amount. In some embodiments, the second agent is an agent for treating cancer, a neurodegenerative disease, a leukodystrophy, an inflammatory disease, a musculoskeletal disease, a metabolic disease, or a disease or disorder associated with impaired function of eIF2B, eIF2α, or a component of the eIF2 pathway or ISR pathway.

The compounds described herein can be used in combination with one another, with other active agents known to be useful in treating cancer, a neurodegenerative disease, an inflammatory disease, a musculoskeletal disease, a metabolic disease, or a disease or disorder associated with impaired function of eIF2B, eIF2α, or a component of the eIF2 pathway or ISR pathway or with adjunctive agents that may not be effective alone, but may contribute to the efficacy of the active agent.

In some embodiments, co-administration includes administering one active agent within 0.5, 1, 2, 4, 6, 8, 10, 12, 16, 20, or 24 hours of a second active agent. Co-administration includes administering two active agents simultaneously, approximately simultaneously (e.g., within about 1, 5, 10, 15, 20, or 30 minutes of each other), or sequentially in any order. In some embodiments, co-administration can be accomplished by co-formulation, i.e., preparing a single pharmaceutical composition including both active agents. In other embodiments, the active agents can be formulated separately. In another embodiment, the active and/or adjunctive agents may be linked or conjugated to one another. In some embodiments, the compounds described herein may be combined with treatments for a cancer, a neurodegenerative disease, a leukodystrophy, an inflammatory disease, a musculoskeletal disease, a metabolic disease, or a disease or disorder associated with impaired function of eIF2B, eIF2α, or a component of the eIF2 pathway or ISR pathway.

In embodiments, the second agent is an anti-cancer agent. In embodiments, the second agent is a chemotherapeutic. In embodiments, the second agent is an agent for improving memory. In embodiments, the second agent is an agent for treating a neurodegenerative disease. In embodiments, the second agent is an agent for treating a leukodystrophy. In embodiments, the second agent is an agent for treating vanishing white matter disease. In embodiments, the second agent is an agent for treating childhood ataxia with CNS hypo-myelination. In embodiments, the second agent is an agent for treating an intellectual disability syndrome. In embodiments, the second agent is an agent for treating pancreatic cancer. In embodiments, the second agent is an agent for treating breast cancer. In embodiments, the second agent is an agent for treating multiple myeloma. In embodiments, the second agent is an agent for treating myeloma. In embodiments, the second agent is an agent for treating a cancer of a secretory cell. In embodiments, the second agent is an agent for reducing eIF2α phosphorylation. In embodiments, the second agent is an agent for inhibiting a pathway activated by eIF2α phosphorylation. In embodiments, the second agent is an agent for inhibiting a pathway activated by eIF2α. In embodiments, the second agent is an agent for inhibiting the integrated stress response. In embodiments, the second agent is an anti-inflammatory agent. In embodiments, the second agent is an agent for treating postsurgical cognitive dysfunction. In embodiments, the second agent is an agent for treating traumatic brain injury. In embodiments, the second agent is an agent for treating a musculoskeletal disease. In embodiments, the second agent is an agent for treating a metabolic disease. In embodiments, the second agent is an anti-diabetic agent.

### Anti-Cancer Agents

“Anti-cancer agent” is used in accordance with its plain ordinary meaning and refers to a composition (e.g. compound, drug, antagonist, inhibitor, modulator) having antineoplastic properties or the ability to inhibit the growth or proliferation of cells. In some embodiments, an anti-cancer agent is a chemotherapeutic. In some embodiments, an anti-cancer agent is an agent identified herein having utility in methods of treating cancer. In some embodiments, an anticancer agent is an agent approved by the FDA or similar regulatory agency of a country other than the USA, for treating cancer. Examples of anti-cancer agents include, but are not limited to, MEK (e.g. MEK1, MEK2, or MEK1 and MEK2) inhibitors (e.g. XL518, CI-1040, PD035901, selumetinib/AZD6244, GSK1120212/trametinib, GDC-0973, ARRY-162, ARRY-300, AZD8330, PD0325901, U0126, PD98059, TAK-733, PD318088, AS703026, BAY 869766), alkylating agents (e.g., cyclophosphamide, ifosfamide, chlorambucil, busulfan, melphalan, mechlorethamine, uramustine, thiotepa, nitrosoureas, nitrogen mustards (e.g., mechloroethamine, cyclophosphamide, chlorambucil, meiphalan), ethylenimine and methylmelamines (e.g., hexamethlymelamine, thiotepa), alkyl sulfonates (e.g., busulfan), nitrosoureas (e.g., carmustine, lomusitne, semustine, streptozocin), triazenes (decarbazine), anti-metabolites (e.g., 5-azathioprine, leucovorin, capecitabine, fludarabine, gemcitabine, pemetrexed, raltitrexed, folic acid analog (e.g., methotrexate), or pyrimidine analogs (e.g., fluorouracil, floxouridine, Cytarabine), purine analogs (e.g., mercaptopurine, thioguanine, pentostatin), etc.), plant alkaloids (e.g., vincristine, vinblastine, vinorelbine, vindesine, podophyllotoxin, paclitaxel, docetaxel, etc.), topoisomerase inhibitors (e.g., irinotecan, topotecan, amsacrine, etoposide (VP 16), etoposide phosphate, teniposide, etc.), antitumor antibiotics (e.g., doxorubicin, adriamycin, daunorubicin, epirubicin, actinomycin, bleomycin, mitomycin, mitoxantrone, plicamycin, etc.), platinum-based compounds (e.g. cisplatin, oxaloplatin, carboplatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide), epipodophyllotoxins (e.g., etoposide), antibiotics (e.g., daunorubicin, doxorubicin, bleomycin), enzymes (e.g., L-asparaginase), inhibitors of mitogen-activated protein kinase signaling (e.g. U0126, PD98059, PD184352, PD0325901, ARRY-142886, SB239063, SP600125, BAY 43-9006, wortmannin, or LY294002, Syk inhibitors, mTOR inhibitors, antibodies (e.g., rituxan), gossyphol, genasense, polyphenol E, Chlorofusin, all trans-retinoic acid (ATRA), bryostatin, tumor necrosis factor-related apoptosis-inducing ligand (TRAIL), 5-aza-2′-deoxycytidine, all trans retinoic acid, doxorubicin, vincristine, etoposide, gemcitabine, imatinib (Gleevec®), geldanamycin, 17-N-Allylamino-17-Demethoxygeldanamycin (17-AAG), flavopiridol, LY294002, bortezomib, trastuzumab, BAY 1 1-7082, PKC412, PD184352, 20-epi-1, 25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+mycobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; 06-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylerie conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone Bl; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; zinostatin stimalamer, Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; iimofosine; interleukin II (including recombinant interleukin II, or rlL.sub.2), interferon alfa-2a; interferon alfa-2b; interferon alfa-n1; interferon alfa-n3; interferon beta-la; interferon gamma-1b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazoie; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride, agents that arrest cells in the G2-M phases and/or modulate the formation or stability of microtubules, (e.g. Taxol, i.e. paclitaxel), Taxotere, compounds comprising the taxane skeleton, Erbulozole (i.e. R-55104), Dolastatin 10 (i.e. DLS-10 and NSC-376128), Mivobulin isethionate (i.e. as CI-980), Vincristine, NSC-639829, Discodermolide (i.e. as NVP-XX-A-296), ABT-751 (Abbott, i.e. E-7010), Altorhyrtins (e.g. Altorhyrtin A and Altorhyrtin C), Spongistatins (e.g. Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (i.e. LU-103793 and SC-D-669356), Epothilones (e.g. Epothilone A, Epothilone B, Epothilone C (i.e. desoxyepothilone A or dEpoA), Epothilone D (i.e. KOS-862, dEpoB, and desoxyepothilone B), Epothilone E, Epothilone F, Epothilone B N-oxide, Epothilone A N-oxide, 16-aza-epothilone B, 21-aminoepothilone B (i.e. BMS-310705), 21-hydroxyepothilone D (i.e. Desoxyepothilone F and dEpoF), 26-fluoroepothilone, Auristatin PE (i.e. NSC-654663), Soblidotin (i.e. TZT-1027), LS-4559-P (Pharmacia, i.e. LS-4577), LS-4578 (Pharmacia, i.e. LS-477-P), LS-4477 (Pharmacia), LS-4559 (Pharmacia), RPR-1 12378 (Aventis), Vincristine sulfate, DZ-3358 (Daiichi), FR-182877 (Fujisawa, i.e. WS-9885B), GS-164 (Takeda), GS-198 (Takeda), KAR-2 (Hungarian Academy of Sciences), BSF-223651 (BASF, i.e. ILX-651 and LU-223651), SAH-49960 (Lilly/Novartis), SDZ-268970 (Lilly/Novartis), AM-97 (Armad/Kyowa Hakko), AM-132 (Armad), AM-138 (Armad/Kyowa Hakko), IDN-5005 (Indena), Cryptophycin 52 (i.e. LY-355703), AC-7739 (Ajinomoto, i.e. AVE-8063A and CS-39·HCl), AC-7700 (Ajinomoto, i.e. AVE-8062, AVE-8062A, CS-39-L-Ser.HCl, and RPR-258062A), Vitilevuamide, Tubulysin A, Canadensol, Centaureidin (i.e. NSC-106969), T-138067 (Tularik, i.e. T-67, TL-138067 and TI-138067), COBRA-1 (Parker Hughes Institute, i.e. DDE-261 and WHI-261), H10 (Kansas State University), H16 (Kansas State University), Oncocidin A 1 (i.e. BTO-956 and DIME), DDE-313 (Parker Hughes Institute), Fijianolide B, Laulimalide, SPA-2 (Parker Hughes Institute), SPA-1 (Parker Hughes Institute, i.e. SPIKET-P), 3-IAABU (Cytoskeleton/Mt. Sinai School of Medicine, i.e. MF-569), Narcosine (also known as NSC-5366), Nascapine, D-24851 (Asta Medica), A-105972 (Abbott), Hemiasterlin, 3-BAABU (Cytoskeleton/Mt. Sinai School of Medicine, i.e. MF-191), TMPN (Arizona State University), Vanadocene acetylacetonate, T-138026 (Tularik), Monsatrol, Inanocine (i.e. NSC-698666), 3-IAABE (Cytoskeleton/Mt. Sinai School of Medicine), A-204197 (Abbott), T-607 (Tularik, i.e. T-900607), RPR-115781 (Aventis), Eleutherobins (such as Desmethyleleutherobin, Desaetyleleutherobin, 1soeleutherobin A, and Z-Eleutherobin), Caribaeoside, Caribaeolin, Halichondrin B, D-64131 (Asta Medica), D-68144 (Asta Medica), Diazonamide A, A-293620 (Abbott), NPI-2350 (Nereus), Taccalonolide A, TUB-245 (Aventis), A-259754 (Abbott), Diozostatin, (−)-Phenylahistin (i.e. NSCL-96F037), D-68838 (Asta Medica), D-68836 (Asta Medica), Myoseverin B, D-43411 (Zentaris, i.e. D-81862), A-289099 (Abbott), A-318315 (Abbott), HTI-286 (i.e. SPA-110, trifluoroacetate salt) (Wyeth), D-82317 (Zentaris), D-82318 (Zentaris), SC-12983 (NCI), Resverastatin phosphate sodium, BPR-OY-007 (National Health Research Institutes), and SSR-25041 1 (Sanofi), steroids (e.g., dexamethasone), finasteride, aromatase inhibitors, gonadotropin-releasing hormone agonists (GnRH) such as goserelin or leuprolide, adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), immunostimulants (e.g., Bacillus Calmette-Guerin (BCG), levamisole, interleukin-2, alpha-interferon, etc.), monoclonal antibodies (e.g., anti-CD20, anti-HER2, anti-CD52, anti-HLA-DR, and anti-VEGF monoclonal antibodies), immunotoxins (e.g., anti-CD33 monoclonal antibody-calicheamicin conjugate, anti-CD22 monoclonal antibody-pseudomonas exotoxin conjugate, etc.), radioimmunotherapy (e.g., anti-CD20 monoclonal antibody conjugated to ^{U1}In, ⁹⁰Y, or ¹³¹I, etc.), triptolide, homoharringtonine, dactinomycin, doxorubicin, epirubicin, topotecan, itraconazole, vindesine, cerivastatin, vincristine, deoxyadenosine, sertraline, pitavastatin, irinotecan, clofazimine, 5-nonyloxytryptamine, vemurafenib, dabrafenib, erlotinib, gefitinib, EGFR inhibitors, epidermal growth factor receptor (EGFR)-targeted therapy or therapeutic (e.g. gefitinib (Iressa™), erlotinib (Tarceva™), cetuximab (Erbitux™), lapatinib (Tykerb™), panitumumab (Vectibix™) vandetanib (Caprelsa™), afatinib/BIBW2992, CI-1033/canertinib, neratinib/HKI-272, CP-724714, TAK-285, AST-1306, ARRY334543, ARRY-380, AG-1478, dacomitinib/PF299804, OSI-420/desmethyl erlotinib, AZD8931, AEE788, pelitinib/EKB-569, CUDC-101, WZ8040, WZ4002, WZ3146, AG-490, XL647, PD153035, BMS-599626), sorafenib, imatinib, sunitinib, dasatinib, or the like.

“Chemotherapeutic” or “chemotherapeutic agent” is used in accordance with its plain ordinary meaning and refers to a chemical composition or compound having antineoplastic properties or the ability to inhibit the growth or proliferation of cells.

Additionally, the compounds described herein can be co-administered with conventional immunotherapeutic agents including, but not limited to, immunostimulants (e.g., Bacillus Calmette-Guerin (BCG), levamisole, interleukin-2, alpha-interferon, etc.), monoclonal antibodies (e.g., anti-CD20, anti-HER2, anti-CD52, anti-HLA-DR, and anti-VEGF monoclonal antibodies), immunotoxins (e.g., anti-CD33 monoclonal antibody-calicheamicin conjugate, anti-CD22 monoclonal antibody-pseudomonas exotoxin conjugate, etc.), and radioimmunotherapy (e.g., anti-CD20 monoclonal antibody conjugated to ^{m}In, ⁹⁰Y, or ¹³¹I, etc.).

In a further embodiment, the compounds described herein can be co-administered with conventional radiotherapeutic agents including, but not limited to, radionuclides such as ⁴⁷Sc, ⁶⁴Cu, ⁶⁷Cu, ⁸⁹Sr, ⁸⁶Y, ⁸⁷Y, ⁹⁰Y, ¹⁰⁵Rh, ^{m}Ag, ^{m}In, ^{117m}Sn, ¹⁴⁹Pm, ¹⁵³Sm, ¹⁶⁶Ho, ¹⁷⁷Lu, ¹⁸⁶Re, ¹⁸⁸Re, ²¹¹At, and ²¹²Bi, optionally conjugated to antibodies directed against tumor antigens.

### Additional Agents

In some embodiments, the second agent for use in combination with a compound (e.g., a compound of Formula (I)) or composition thereof described herein is an agent for use in treating a neurodegenerative disease, a leukodystrophy, an inflammatory disease, a musculoskeletal disease, or a metabolic disease. In some embodiments, a second agent for use in combination with a compound (e.g., a compound of Formula (I)) or composition thereof described herein is an agent approved by the FDA or similar regulatory agency of a country other than the USA, for treating a disease, disorder, or condition described herein.

In some embodiments, a second agent for use in treating a neurodegenerative disease, a leukodystrophy, an inflammatory disease, a musculoskeletal disease, or a metabolic disease includes, but is not limited to, an anti-psychotic drug, anti-depressive drug, anti-anxiety drug, analgesic, a stimulant, a sedative, a pain reliever, an anti-inflammatory agent, a benzodiazepine, a cholinesterase inhibitor, a non-steroidal anti-inflammatory drug (NSAID), a corticosteroid, a MAO inhibitor, a beta-blocker, a calcium channel blocker, an antacid, or other agent. Exemplary second agents may include donepezil, galantamine, rivastigmine, memantine, levodopa, dopamine, pramipexole, ropinirole, rotigotine, doxapram, oxazepam, quetiapine, selegiline, rasagiline, entacapone, benztropine, trihexyphenidyl, riluzole, diazepam, chlorodiazepoxide, lorazepam, alprazolam, buspirone, gepirone, ispapirone, hydroxyzine, propranolol, hydroxyzine, midazolam, trifluoperazine, methylphenidate, atomoxetine, methylphenidate, pemoline, perphenazine, divalproex, valproic acid, sertraline, fluoxetine, citalopram, escitalopram, paroxetine, fluvoxamine, trazodone, desvenlafaxine, duloxetine, venlafaxine, amitriptyline, amoxapine, clomipramine, desipramine, imipramine, nortriptyline, protriptyline, trimipramine, maprotiline, bupropion, nefazodone, vortioxetine, lithium, clozapine, fluphenazine, haloperidol, paliperidone, loxapine, thiothixene, pimozide, thioridazine, risperidone, aspirin, ibuprofen, naproxen, acetaminophen, azathioprine, methotrexate, mycophenolic acid, leflunomide, dibenzoylmethane, cilostazol, pentoxifylline, duloxetine, a cannabinoid (e.g., nabilone), simethicone, magaldrate, aluminum salts, calcium salts, sodium salts, magnesium salts, alginic acid, acarbose, albiglutide, alogliptin, metformin, insulin, lisinopril, atenolol, atorvastatin, fluvastatin, lovastatin, pitavastatin, simvastatin, rosuvastatin, and the like.

Naturally derived agents or supplements may also be used in conjunction with a compound of Formula (I) or a composition thereof to treat a neurodegenerative disease, an inflammatory disease, a musculoskeletal disease, or a metabolic disease. Exemplary naturally derived agents or supplements include omega-3 fatty acids, carnitine, citicoline, curcumin, gingko, vitamin E, vitamin B (e.g., vitamin B5, vitamin B6, or vitamin B12), huperzine A, phosphatidylserine, rosemary, caffeine, melatonin, chamomile, St. John's wort, tryptophan, and the like.

### EXAMPLES

In order that the invention described herein may be more fully understood, the following examples are set forth. The synthetic and biological examples described in this application are offered to illustrate the compounds, pharmaceutical compositions, and methods provided herein and are not to be construed in any way as limiting their scope.

### Synthetic Protocols

The compounds provided herein can be prepared from readily available starting materials using modifications to the specific synthesis protocols set forth below that would be well known to those of skill in the art. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvents used, but such conditions can be determined by those skilled in the art by routine optimization procedures. General scheme relating to methods of making exemplary compounds of the invention are additionally described in the section entitled Methods of Making Compounds.

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are well known in the art. For example, numerous protecting groups, and their introduction and removal, are described in Greene et al., Protecting Groups in Organic Synthesis, Second Edition, Wiley, New York, 1991, and references cited therein.

### Abbreviations

APCI for atmospheric pressure chemical ionization; COMU for (1-cyano-2-ethoxy-2-oxoethylidenaminooxy)dimethylamino-morpholino-carbenium hexafluorophosphate; DCI for desorption chemical ionization; DMSO for dimethyl sulfoxide; ESI for electrospray ionization; HATU for 1-[bis(dimethylamino)methylene]-1H-1,2,3-triazolo[4,5-b]pyridinium 3-oxid hexafluorophosphate; HPLC for high performance liquid chromatography; LC/MS for liquid chromatography/mass spectrometry; MS for mass spectrum; NMR for nuclear magnetic resonance; psi for pounds per square inch; SCX for strong cation exchange; SFC for supercritical fluid chromatography; T3P for 2,4,6-tripropyl-1,3,5,2,4,6-trioxatriphosphinane 2,4,6-trioxide; TEMPO for 2,2,6,6-tetramethyl-1-piperidinyloxy, free radical; TLC for thin-layer chromatography; and UV for ultraviolet.

### Example 1: N-{4-[2-(4-chloro-3-fluorophenoxy)acetamido]bicyclo[2.2.2]octan-1-yl}-2H-1,3-benzodioxole-2-carboxamide (Compound 100)

### Example 1A: tert-butyl (4-aminobicyclo[2.2.2]octan-1-yl)carbamate

Bicyclo[2.2.2]octane-1,4-diamine dihydrochloride (PharmaBlock, 200 mg, 1.43 mmol) was dissolved in methanol (5 mL). The solution was basified with 50% aqueous sodium hydroxide. After stirring for 15 minutes (slight exotherm), the mixture was diluted with water and brine and extracted with dichloromethane (3×150 mL). The combined organic layers were dried (Na₂SO₄) and filtered. The filtrate was concentrated under reduced pressure to give the free base as a white solid. The free base, bicyclo[2.2.2]octane-1,4-diamine (176 mg, 1.255 mmol), di-tert-butyl dicarbonate (274 mg, 1.255 mmol), and tetrahydrofuran (100 mL) were stirred at ambient temperature for 17 hours. The reaction mixture was concentrated under reduced pressure, and the residue was partitioned between ethyl acetate and aqueous sodium carbonate. The organic layer was washed with brine, then dried (MgSO₄) and filtered. The filtrate was concentrated under reduced pressure to provide the title intermediate as an off-white solid (258 mg, 86% yield). ¹H NMR (methanol-d₄) δ ppm 1.91-1.85 (m, 7H), 1.65-1.60 (m, 2H), 1.40 (s, 12H); MS (DCI-NH₃) m/z 241 (M+H)⁺.

### Example 1B: tert-butyl (4-(2-(4-chloro-3-fluorophenoxy)acetamido)bicyclo[2.2.2]octan-1-yl)carbamate

A 50 mL round bottom flask, equipped with a magnetic stir bar, was charged with 2-(4-chloro-3-fluorophenoxy)acetic acid (234 mg, 1.144 mmol), the product of Example 1A (250 mg, 1.040 mmol), and (1-cyano-2-ethoxy-2-oxoethylidenaminooxy)dimethylamino-morpholino-carbenium hexafluorophosphate (COMU®, 535 mg, 1.248 mmol). The flask contents were placed under a dry nitrogen atmosphere and N,N-dimethylformamide (4 mL) was introduced via syringe. The reaction mixture was then stirred at ambient temperature as N,N-diisopropylethylamine (0.545 mL, 3.12 mmol) was added dropwise via syringe. The reaction mixture was stirred at ambient temperature for 19 hours. The reaction mixture was diluted with water (pH=10). An insoluble beige solid was collected by filtration and rinsed thoroughly with water. The material was purified by column chromatography on an Analogix® IntelliFlash™-310 (Isco RediSep® 40 g silica gel cartridge, 70:30 to 0:100 heptane/ethyl acetate). Fractions were combined and concentrated under reduced pressure to give the title intermediate as a white solid (69.5 mg, 15.65% yield). ¹H NMR (CDCl₃) δ ppm 7.31 (t, J=8.6 Hz, 1H), 6.73 (dd, J=10.3, 2.9 Hz, 1H), 6.64 (ddd, J=8.9, 2.9, 1.2 Hz, 1H), 6.07 (s, 1H), 4.32 (s, 1H), 4.31 (s, 2H), 2.05-1.91 (m, 12H), 1.42 (s, 9H); MS (ESI+) m/z 426 (M+H)⁺, m/z 853 (2M+H)⁺; MS (ESI−) m/z 425 (M−H)⁻.

### Example 1C: N-(4-aminobicyclo[2.2.2]octan-1-yl)-2-(4-chloro-3-fluorophenoxy)acetamide, trifluoroacetic Acid

Trifluoroacetic acid (1 mL, 12.98 mmol) was added to a solution of the product of Example 1B (158 mg, 0.37 mmol) in dichloromethane (2.0 mL). The resulting mixture was stirred at ambient temperature for 30 minutes and then concentrated under reduced pressure to give the title compound (0.16 g, 0.36 mmol, 98% yield). MS (ESI⁺) m/z 327 (M+H)⁺.

### Example 1D: N-{4-[2-(4-chloro-3-fluorophenoxy)acetamido]bicyclo[2.2.2]octan-1-yl}-2H-1,3-benzodioxole-2-carboxamide

1-[Bis(dimethylamino)methylene]-1H-1,2,3-triazolo[4,5-b]pyridinium 3-oxid hexafluorophosphate (HATU, 30.4 mg, 0.08 mmol) was added to a solution of 1,3-benzodioxole-2-carboxylic acid (12.1 mg, 0.07 mmol, Matrix), the product of Example 1C (32 mg, 0.073 mmol) and triethylamine (0.051 mL, 0.36 mmol) in N,N-dimethylformamide (2.0 mL). The resulting solution was stirred at ambient temperature for 1 hour, filtered through a glass microfiber frit and purified by preparative HPLC [YMC TriArt™ C18 Hybrid 5 μm column, 50×100 mm, flow rate 70 mL/minute, 5-100% gradient of acetonitrile in buffer (0.025 M aqueous ammonium bicarbonate, adjusted to pH 10 with ammonium hydroxide)] to give the title compound (12 mg, 0.025 mmol, 35% yield). ¹H NMR (400 MHz, DMSO-d₆) δ ppm 8.09 (s, 1H), 7.52-7.44 (m, 2H), 7.02 (dd, J=11.4, 2.9 Hz, 1H), 6.93-6.88 (m, 2H), 6.86-6.78 (m, 3H), 6.25 (s, 1H), 4.43 (s, 2H), 1.91 (br s, 12H); MS (ESI⁺) m/z 475 (M+H)⁺.

### Example 2: 2-(5-chloro-2,3-dihydro-1H-indol-1-yl)-N-{(3S)-4-[2-(4-chloro-3-fluorophenoxy)acetamido]-3-hydroxybicyclo[2.2.2]octan-1-yl}acetamide (Compound 101)

### Example 2A: ethyl 1,4-dioxaspiro[4.5]decane-8-carboxylate

A mixture of ethyl 4-oxocyclohexanecarboxylate (11.70 mL, 73.4 mmol), ethane-1,2-diol (12.29 mL, 220 mmol), and p-toluenesulfonic acid monohydrate (1.397 g, 7.34 mmol) in toluene (200 mL) was stirred at reflux with a Dean-Stark trap apparatus for 180 minutes. The reaction mixture was neutralized with N-ethyl-N-isopropylpropan-2-amine and then concentrated. The residue was purified on silica gel (0-30% ethyl acetate in heptane) to give 12.77 g of the title compound. ¹H NMR (400 MHz, DMSO-d₆) δ ppm 4.01 (q, J=7.1 Hz, 2H), 3.81 (s, 4H), 2.32 (tt, J=10.4, 3.8 Hz, 1H), 1.83-1.71 (m, 2H), 1.66-1.57 (m, 1H), 1.62-1.38 (m, 5H), 1.13 (t, J=7.1 Hz, 3H).

### Example 2B: ethyl 8-acetyl-1,4-dioxaspiro[4.5]decane-8-carboxylate

To a solution of diisopropylamine (5.19 mL, 36.4 mmol) in tetrahydrofuran (25 mL) at 0° C. was added n-butyllithium slowly below 5° C. After stirring for 30 minutes, the solution was cooled to −78° C. under nitrogen, and a solution of Example 2A (6.0 g, 28.0 mmol) in tetrahydrofuran (3 mL) was added slowly, and the resultant mixture was stirred for 30 minutes at the same temperature. Then acetyl chloride (2.59 mL, 36.4 mmol) was added slowly to maintain the temperature below −60° C., and the mixture was stirred at −70° C. for 2 hours. The reaction was quenched with saturated NH₄Cl solution, and the aqueous phase was extracted with ethyl acetate. The organic layer was washed with brine, dried over magnesium sulfate and filtered. The filtrate was concentrated, and the residue was purified on silica gel (0-70% ethyl acetate in heptane) to give 6.78 g of the title compound. ¹H NMR (500 MHz, DMSO-d₆) δ ppm 4.19-4.11 (m, 2H), 3.85 (s, 4H), 2.13 (s, 3H), 2.10-2.01 (m, 2H), 1.90 (ddd, J=13.9, 9.6, 4.6 Hz, 2H), 1.54 (th, J=13.6, 4.7 Hz, 4H), 1.18 (dd, J=7.6, 6.5 Hz, 3H).

### Example 2C: ethyl 1-acetyl-4-oxocyclohexane-1-carboxylate

A mixture of Example 2B (6.5 g, 25.4 mmol) and HCl (21.13 mL, 127 mmol) in acetone (60 mL) was stirred at ambient temperature overnight. Volatiles were removed under reduced pressure, and the residue was partitioned between water and dichloromethane. The organic layer was washed with brine, dried over magnesium sulfate and filtered. The filtrate was concentrated to give 5.46 g of the title compound that was used without further purification. ¹H NMR (400 MHz, DMSO-d₆) δ ppm 4.16 (q, J=7.1 Hz, 2H), 2.17 (s, 3H), 2.35-2.07 (m, 8H), 1.17 (t, J 7.1 Hz, 3H).

### Example 2D: ethyl 4-(benzylamino)-2-oxobicyclo[2.2.2]octane-1-carboxylate, hydrochloric Acid

A mixture of Example 2C (9.7 g, 45.7 mmol), benzylamine (14.98 mL, 137 mmol), and p-toluenesulfonic acid monohydrate (0.087 g, 0.457 mmol) in toluene (100 mL) was stirred at reflux with a Dean-Stark trap apparatus overnight. The mixture was concentrated, and the residue was stirred with a mixture of ethyl acetate (50 mL) and 3 N HCl (100 mL) for 30 minutes. The precipitate was collected by filtration, washed with mixture of ethyl acetate/heptane, and air-dried to give 11.3 g of title compound as an HCl salt. The filtrate was neutralized with 6 N NaOH and extracted with ethyl acetate (100 mL×2). The organic layer was washed with brine, dried over magnesium sulfate and filtered. The residue was purified on silica gel (0-70% ethyl acetate in heptane) to give another 0.77 g of the title compound. ¹H NMR (400 MHz, DMSO-d₆) δ ppm 9.73 (t, J=6.2 Hz, 2H), 7.87-7.12 (m, 5H), 4.09 (m, 4H), 2.88 (s, 2H), 2.08 (dt, J=20.7, 13.4 Hz, 6H), 1.16 (t, J=7.1 Hz, 3H); MS (ESI⁺) m/z 302.1 (M+H)⁺.

### Example 2E: 4-(benzylamino)-2-oxobicyclo[2.2.2]octane-1-carboxylic Acid hydrochloride

A mixture of 2D (20.7 g, 61.3 mmol) and 25% aqueous sodium hydroxide (49.0 mL, 306 mmol) in methanol (200 mL) and water (200 mL) was stirred for 24 hours at ambient temperature. The mixture was concentrated, and the residue was acidified with 1 N HCl. The precipitate was collected by filtration, washed with water, and air dried to give 16.4 g of the title compound. ¹H NMR (400 MHz, DMSO-d₆) δ ppm 12.70 (s, 1H), 9.67 (s, 2H), 7.62 (dd, J=7.5, 2.0 Hz, 2H), 7.43 (d, J=6.6 Hz, 3H), 4.13 (s, 2H), 2.87 (s, 2H), 2.08 (tdq, J=14.4, 10.8, 5.8, 5.0 Hz, 8H).

### Example 2F: 1-amino-4-(benzylamino)bicyclo[2.2.2]octan-2-one, trifluoroacetic Acid

To a mixture of Example 2E (5.0 g, 16.14 mmol) and oxalyl dichloride (24.21 mL, 48.4 mmol) in dichloromethane (100 mL) was added N,N-dimethylformamide (0.250 mL, 3.23 mmol), and the suspension was stirred at ambient temperature for 14 hours. The mixture was concentrated, and the residue was triturated with ether/heptane. The precipitate was collected by filtration and dried to give 4.99 g of 4-(benzylamino)-2-oxobicyclo[2.2.2]octane-1-carbonyl chloride hydrochloride which was used in next step without further purification. To a mixture of sodium azide (0.832 g, 12.80 mmol) in dioxane (10 mL) and water (10 mL) at 0° C. was added a suspension of the crude 4-(benzylamino)-2-oxobicyclo[2.2.2]octane-1-carbonyl chloride hydrochloride (0.934 g, 3.2 mmol) in dioxane (30 mL), and the solution was stirred at ambient temperature for 30 minutes. Volatiles were removed to give 4-(benzylamino)-2-oxobicyclo[2.2.2]octane-1-carbonyl azide which was suspended with 50 mL of toluene and heated at 65° C. for 2 hours to convert to the corresponding isocyanate. Then 3 N HCl (40 mL) was added carefully, and the mixture was stirred at 100° C. for 3 hours. Volatiles were removed under vacuum, and the residue was stirred with methanol and the inorganic salts were removed by filtration. The filtrate was concentrated, and the residue was purified by HPLC (0-60% acetonitrile in 0.1% trifluoroacetic acid/water on a Phenomenex® C18 10 μm (250 mm×50 mm) column at a flowrate of 50 mL/minute) to give 550 mg of the title compound. ¹H NMR (400 MHz, DMSO-d₆) δ ppm 9.47 (s, 2H), 8.59 (s, 3H), 7.55-7.39 (m, 5H), 4.18 (s, 2H), 3.01 (s, 2H), 2.28-2.09 (m, 6H), 1.96 (td, J=12.6, 12.0, 7.0 Hz, 2H); MS (ESI⁺) m/z 245.1 (M+H)⁺.

### Example 2G: N-[4-(benzylamino)-2-oxobicyclo[2.2.2]octan-1-yl]-2-(4-chloro-3-fluorophenoxy)acetamide

A mixture of Example 2F (0.66 g, 0.699 mmol), 2-(4-chloro-3-fluorophenoxy)acetic acid (0.179 g, 0.873 mmol) and N-ethyl-N-isopropylpropan-2-amine (0.610 mL, 3.49 mmol) in N,N-dimethylformamide (10 mL) was treated with 2-(3H-[1,2,3]triazolo[4,5-b]pyridin-3-yl)-1,1,3,3-tetramethylisouronium hexafluorophosphate (V) (0.398 g, 1.048 mmol), and the reaction mixture was stirred at ambient temperature for 15 minutes. The reaction mixture was partitioned between water and dichloromethane. The organic layer was concentrated, and the residue was purified by HPLC (15-100% acetonitrile in 0.1% trifluoroacetic acid/water on a Phenomenex® C18 10 μm (250 mm×50 mm) column at a flowrate of 50 mL/minute) to give 0.34 g of the title compound. ¹H NMR (400 MHz, DMSO-d₆) δ ppm 9.23 (d, J=6.6 Hz, 2H), 7.84 (s, 1H), 7.55-7.39 (m, 6H), 7.09 (dd, J=11.4, 2.9 Hz, 1H), 6.86 (ddd, J=8.9, 2.8, 1.2 Hz, 1H), 4.59 (s, 2H), 4.17 (t, J=5.6 Hz, 2H), 2.90 (d, J=3.7 Hz, 2H), 2.50-2.36 (m, 2H), 2.23-2.09 (m, 2H), 2.13-1.95 (m, 4H); MS (ESI⁺) m/z 431.2 (M+H)⁺.

### Example 2H: N-(4-amino-2-oxobicyclo[2.2.2]octan-1-yl)-2-(4-chloro-3-fluorophenoxy)acetamide, trifluoroacetic Acid

To a mixture of Pd(OH)₂ (2.7 g, 3.85 mmol) in tetrahydrofuran (500 mL) was added Example 2G (10 g, 22.05 mmol) under argon at ambient temperature, and the reaction mixture was stirred for 7.5 hours under H2 at 50 psi. Methanol (1000 mL) was added, and the mixture was filtered through a pad of diatomaceous earth. The filter cake was washed with methanol (1000 mL), and the filtrate was concentrated under reduced pressure. The residue was purified by reversed phase HPLC (10-80% acetonitrile in 0.075% trifluoroacetic acid/water over 30 minutes on a 250 mm×80 mm Phenomenex® Luna®-C18 10 μm column at a flowrate of 80 mL/minute) to give the title compound. ¹H NMR (400 MHz, DMSO-d₆) δ ppm 8.49 (s, 3H), 7.81 (s, 1H), 7.49 (t, J=8.8 Hz, 1H), 7.08 (dd, J=11.3, 2.6 Hz, 1H), 6.85 (dd, J=8.9, 2.6 Hz, 1H), 4.58 (s, 2H), 2.73 (s, 2H), 2.38 (t, J=9.1 Hz, 2H), 1.95 (d, J=8.3 Hz, 6H).

### Example 2I: N-(4-amino-2-hydroxybicyclo[2.2.2]octan-1-yl)-2-(4-chloro-3-fluorophenoxy)acetamide hydrochloride

A mixture of Example 2H (7 g, 15.39 mmol) and NaBH₄ (0.582 g, 15.39 mmol) in a mixture of methanol (200 mL) and methylene chloride (200 mL) was stirred at 20° C. for 12 hours. The solution was concentrated, and the residue was purified by preparative HPLC (5˜100% acetonitrile in water with 0.05% HCl on a SNAP C18 (20-35 μm, 800 g) column at a flow rate of 200 mL/minute) to provide the title compound (5.0 g, 83%); MS (ESI⁺) m/z 343.1 (M+H)⁺.

### Example 2J. N-[(2S)-4-amino-2-hydroxybicyclo[2.2.2]octan-1-yl]-2-(4-chloro-3-fluorophenoxy)acetamide

The title compound was isolated by chiral preparative SFC of Example 21 as the first peak eluted off the column. The chirality of its enantiomer (the 2nd peak off the column) was confirmed by X-ray crystallography. The preparative SFC (Supercritical Fluid Chromatography) was performed on a Thar 200 preparative SFC (SFC-5) system using a Chiralpak® IC-H, 250×30 mm I.D., 5 μm column. The column was heated at 38° C., and the backpressure regulator was set to maintain 100 bar. The mobile phase A was CO₂ and B was isopropanol (0.1% ammonium hydroxide). The eluent was held isocratically at 40% of mobile phase B at a flowrate of 75 mL/minute. Fraction collection was time triggered with UV monitor wavelength set at 220 nm. MS (ESI⁺) m/z 343.1 (M+H)⁺.

### Example 2K: (S)-2-chloro-N-(4-(2-(4-chloro-3-fluorophenoxy)acetamido)-3-hydroxybicyclo[2.2.2]octan-1-yl)acetamide

A mixture of Example 2J (1000 mg, 2.92 mmol), 2-chloroacetic acid (317 mg, 3.35 mmol) and N-ethyl-N-isopropylpropan-2-amine (1.783 mL, 10.21 mmol) in N,N-dimethylformamide (5 mL) was treated with 2-(3H-[1,2,3]triazolo[4,5-b]pyridin-3-yl)-1,1,3,3-tetramethylisouronium hexafluorophosphate (V) (1387 mg, 3.65 mmol), and the reaction mixture was stirred at room temperature for 30 minutes. Volatiles were removed, and the residue was purified by HPLC (Phenomenex® Luna® C18(2) 10 μm 100 Å AXIA™ column (250 mm×50 mm). A 30-100% gradient of acetonitrile (A) and 0.1% trifluoroacetic acid in water (B) is used over 25 minutes, at a flow rate of 50 mL/minute) to give 780 mg of the title compound. ¹H NMR (400 MHz, DMSO-d₆) δ ppm 7.79 (s, 1H), 7.54 (t, J=8.9 Hz, 1H), 7.32 (s, 1H), 7.11 (dd, J=11.4, 2.9 Hz, 1H), 6.89 (ddd, J=8.9, 2.9, 1.2 Hz, 1H), 5.14 (d, J=4.4 Hz, 1H), 4.53 (s, 2H), 4.10 (dt, J=8.8, 3.7 Hz, 1H), 3.99 (s, 2H), 2.30 (ddd, J=12.6, 9.4, 2.4 Hz, 1H), 2.12 (ddd, J=12.3, 10.4, 5.0 Hz, 1H), 2.01-1.75 (m, 8H); MS (ESI⁺) m/z 419.0 (M+H)⁺.

### Example 2L: 2-(5-chloro-2,3-dihydro-1H-indol-1-yl)-N-{(3S)-4-[2-(4-chloro-3-fluorophenoxy)acetamido]-3-hydroxybicyclo[2.2.2]octan-1-yl}acetamide

A mixture of Example 2K (50.0 mg, 0.119 mmol) in acetone (1.0 mL) was treated with potassium carbonate (33.0 mg, 0.239 mmol), 5-chloroindoline (36.6 mg, 0.239 mmol) and potassium iodide (1.386 mg, 8.35 μmol), and the mixture was stirred at 140° C. for 45 minutes in Biotage® Initiator microwave reactor (0-450 W). The reaction mixture was concentrated, and the residue was purified by HPLC (Phenomenex® Luna® C18(2) 10 μm 100 Å AXIA™ column (250 mm×50 mm). A 30-100% gradient of acetonitrile (A) and 0.1% trifluoroacetic acid in water (B) is used over 25 minutes, at a flow rate of 50 mL/minute) to give 31 mg of the title compound. ¹H NMR (400 MHz, DMSO-d₆) δ ppm 7.48 (t, J=8.9 Hz, 1H), 7.39 (s, 1H), 7.24 (s, 1H), 7.11-7.00 (m, 2H), 6.97 (dd, J=8.3, 2.3 Hz, 1H), 6.82 (ddd, J=9.0, 3.0, 1.2 Hz, 1H), 6.38 (d, J=8.3 Hz, 1H), 4.46 (s, 2H), 4.06-3.94 (m, 1H), 3.42 (d, J=8.4 Hz, 2H), 2.90 (t, J=8.4 Hz, 2H), 2.25 (ddd, J=12.4, 9.4, 2.4 Hz, 1H), 2.05 (ddd, J=12.2, 10.3, 5.0 Hz, 1H), 1.91 (d, J=8.8 Hz, 2H), 1.88-1.79 (m, 1H), 1.75 (ddt, J=13.2, 10.3, 3.2 Hz, 5H); MS (ESI⁺) m/z 536.4 (M+H)⁺.

### Example 3: (2R)-6-chloro-N-{3-[2-(4-chloro-3-fluorophenoxy)acetamido]bicyclo[1.1.1]pentan-1-yl}-3,4-dihydro-2H-1,4-benzoxazine-2-carboxamide (Compound 102)

Example 14 was purified by chiral SFC (supercritical fluid chromatography) using a (S,S) Whelk-O®1 column (20×250 mm, 5 micron) eluted with 30% CH₃OH in CO₂ at 28° C. with a CO₂ flow rate of 56 mL/minute, CH₃OH flow rate of 24 mL/minute, front pressure of 179 bar, and back pressure of 100 bar to give the title compound (second enantiomer eluted, 0.016 g, 0.032 mmol, 40% yield). The absolute stereochemistry of this title compound was arbitrarily assigned. ¹H NMR (400 MHz, DMSO-d₆) δ ppm 8.71 (s, 1H), 8.63 (s, 1H), 7.49 (t, J=8.9 Hz, 1H), 7.07 (dd, J=11.3, 2.8 Hz, 1H), 6.92-6.82 (m, 1H), 6.77 (d, J=8.5 Hz, 1H), 6.60 (d, J=2.5 Hz, 1H), 6.51 (dd, J=8.5, 2.5 Hz, 1H), 6.15 (s, 1H), 4.47 (s, 2H), 4.42 (dd, J=7.4, 2.9 Hz, 1H), 3.23-3.10 (m, 2H), 2.26 (s, 6H); MS (ESI+) m/z 480 (M+H)⁺.

### Example 4: (2S)-6-chloro-N-{3-[2-(4-chloro-3-fluorophenoxy)acetamido]bicyclo[1.1.1]pentan-1-yl}-3,4-dihydro-2H-1,4-benzoxazine-2-carboxamide (Compound 103)

Example 14 was purified by chiral SFC (supercritical fluid chromatography) using a (S,S) Whelk-O®1 column (20×250 mm, 5 micron) eluted with 30% CH₃OH in CO₂ at 28° C. with a CO₂ flow rate of 56 mL/minute, CH₃OH flow rate of 24 mL/minute, front pressure of 179 bar, and back pressure of 100 bar to give the title compound (first enantiomer eluted, 0.015 g, 0.031 mmol, 39% yield). The absolute stereochemistry of this title compound was arbitrarily assigned. ¹H NMR (400 MHz, DMSO-d₆) δ ppm 8.71 (s, 1H), 8.63 (s, 1H), 7.49 (t, J=8.9 Hz, 1H), 7.07 (dd, J=11.3, 2.8 Hz, 1H), 6.92-6.82 (m, 1H), 6.77 (d, J=8.5 Hz, 1H), 6.60 (d, J=2.5 Hz, 1H), 6.51 (dd, J=8.5, 2.5 Hz, 1H), 6.15 (s, 1H), 4.47 (s, 2H), 4.42 (dd, J=7.4, 2.9 Hz, 1H), 3.23-3.10 (m, 2H), 2.26 (s, 6H); MS (ESI⁺) m/z 480 (M+H)⁺.

### Example 5: (2S)-6-chloro-N-{3-[2-(4-chloro-3-fluorophenoxy)acetamido]bicyclo[1.1.1]pentan-1-yl}-4-methyl-3,4-dihydro-2H-1,4-benzoxazine-2-carboxamide (Compound 104)

Example 13D was purified by preparative chiral SFC (supercritical fluid chromatography) using a (S,S) Whelk-O®1 column (20×250 mm, 5 micron) eluted with 44% CH₃OH in CO₂ at 33° C. with a CO₂ flow rate of 40 mL/minute, CH₃OH flow rate of 32 mL/minute, front pressure of 192 bar, and back pressure of 100 bar to give the title compound (first enantiomer eluted out of the column, 0.0082 g, 0.017 mmol, 43% yield). The absolute stereochemistry of this title compound was arbitrarily assigned. ¹H NMR (400 MHz, DMSO-d₆) δ ppm 8.71 (s, 1H), 8.67 (s, 1H), 7.49 (t, J=8.9 Hz, 1H), 7.07 (dd, J=11.3, 2.9 Hz, 1H), 6.85 (ddd, J=9.0, 2.9, 1.2 Hz, 1H), 6.80 (d, J=8.4 Hz, 1H), 6.70 (d, J=2.4 Hz, 1H), 6.62 (dd, J=8.5, 2.4 Hz, 1H), 4.61 (dd, J=7.3, 3.0 Hz, 1H), 4.47 (s, 2H), 3.40-3.37 (m, 1H), 3.18 (dd, J=12.0, 7.4 Hz, 1H), 2.83 (s, 3H), 2.26 (s, 6H); MS (ESI⁺) m/z 494 (M+H)⁺.

### Example 6: (2S)—N-{(3S)-4-[2-(4-chloro-3-fluorophenoxy)acetamido]-3-hydroxybicyclo[2.2.2]octan-1-yl}-6-fluoro-3,4-dihydro-2H-1-benzopyran-2-carboxamide (Compound 105)

A mixture of Example 2J (60.0 mg, 0.175 mmol), triethylamine (0.032 mL, 0.228 mmol), (S)-6-fluorochroman-2-carboxylic acid (41.2 mg, 0.210 mmol) and 1-[bis(dimethylamino)methylene]-1H-1,2,3-triazolo[4,5-b]pyridinium 3-oxid hexafluorophosphate (HATU, 80 mg, 0.210 mmol) in N,N-dimethylformamide (2 mL) was stirred overnight. The reaction mixture was quenched with brine and saturated NaHCO₃and extracted with ethyl acetate (2×). The combined organic layers were concentrated, and the residue was purified by reverse-phase HPLC performed on a Zorbax Rx-C18 column (250×21.2 mm, 7 μm particle size) using a gradient of 10% to 95% acetonitrile. 0.1% aqueous trifluoroacetic acid over 30 minutes at a flow rate of 18 mL/minute to provide the title compound (26.9 mg, 30%). ¹H NMR (400 MHz, DMSO-d₆) δ ppm 7.48 (t, J=8.9 Hz, 1H), 7.24 (d, J=13.1 Hz, 2H), 7.05 (dd, J=11.4, 2.9 Hz, 1H), 6.91 (ddd, J=8.4, 4.5, 1.7 Hz, 2H), 6.88-6.79 (m, 2H), 5.07 (s, brd, 1H), 4.47 (s, 2H), 4.41 (dd, J=8.7, 3.0 Hz, 1H), 4.04 (dd, J=9.6, 3.1 Hz, 1H), 2.84-2.60 (m, 2H), 2.27 (ddd, J=12.1, 9.5, 2.1 Hz, 1H), 2.13-2.00 (m, 2H), 1.99-1.71 (m, 9H); MS (ESI⁺) m/z 521.2 (M+H)⁺.

### Example 7: (2R)—N-{(3S)-4-[2-(4-chloro-3-fluorophenoxy)acetamido]-3-hydroxybicyclo[2.2.2]octan-1-yl}-6-fluoro-3,4-dihydro-2H-1-benzopyran-2-carboxamide (Compound 106)

The procedures described in Example 6 substituting (R)-6-fluorochroman-2-carboxylic acid for (S)-6-fluorochroman-2-carboxylic acid gave the title compound. ¹H NMR (400 MHz, DMSO-d₆) δ ppm 7.48 (t, J=8.9 Hz, 1H), 7.24 (d, J=12.5 Hz, 2H), 7.05 (dd, J=11.4, 2.9 Hz, 1H), 6.96-6.88 (m, 2H), 6.88-6.78 (m, 2H), 4.47 (s, 2H), 4.45-4.38 (m, 1H), 4.04 (dd, J=9.7, 3.1 Hz, 1H), 2.84-2.58 (m, 2H), 2.28 (ddd, J=12.3, 9.5, 2.3 Hz, 1H), 2.06 (dtq, J=7.8, 5.8, 3.4, 2.3 Hz, 2H), 1.98-1.67 (m, 9H); MS (ESI⁺) m/z 521.2 (M+H)⁺.

### Example 8: 6-chloro-N-{(3S)-4-[2-(4-chloro-3-fluorophenoxy)acetamido]-3-hydroxybicyclo[2.2.2]octan-1-yl}-4-methyl-3,4-dihydro-2H-1,4-benzoxazine-2-carboxamide (Compound 107)

### Example 8A: ethyl 6-chloro-4-methyl-3,4-dihydro-2H-benzo[b][1,4]oxazine-2-carboxylate

A mixture of ethyl 6-chloro-3,4-dihydro-2H-benzo[b][1,4]oxazine-2-carboxylate (0.450 g, 1.862 mmol), potassium carbonate (0.772 g, 5.59 mmol), and iodomethane (0.349 mL, 5.59 mmol) in acetone (4 mL) was heated at reflux in a capped vial overnight. More iodomethane (0.349 mL) was added, and the mixture was heated at reflux for 5 hours. After cooling, the solid was filtered. The filtrate was concentrated, and the residue was treated with water and extracted with ethyl acetate (2×). The combined organic layers were dried over MgSO₄, filtered, and concentrated. The residue was purified on a 40 g column using a Biotage® Isolera™ One flash system eluted with heptanes/ethyl acetate (9:1 to 8:2) to provide the title compound (0.308 g, 65%). MS (ESI⁺) m/z 256.1 (M+H)⁺.

### Example 8B: 6-chloro-4-methyl-3,4-dihydro-2H-benzo[b][1,4]oxazine-2-carboxylic acid

A solution of Example 8A (0.300 g, 1.173 mmol) in tetrahydrofuran (3 mL) and methanol (2 mL) was treated with a solution of lithium hydroxide (0.084 g, 3.52 mmol) in water (1.5 mL). The mixture was stirred for 4 hours. The reaction mixture was concentrated. The concentrate was dissolved in water and acidified with 5% citric acid until pH=4. The precipitate was collected by filtration, rinsed with water, and vacuum oven-dried to provide the title compound (0.171 g, 64%). MS (ESI⁺) m/z 228.2 (M+H)⁺.

### Example 8C: 6-chloro-N-{(3S)-4-[2-(4-chloro-3-fluorophenoxy)acetamido]-3-hydroxybicyclo[2.2.2]octan-1-yl}-4-methyl-3,4-dihydro-2H-1,4-benzoxazine-2-carboxamide

A mixture of Example 2J (110.0 mg, 0.321 mmol), triethylamine (0.058 mL, 0.417 mmol), Example 8B (88 mg, 0.385 mmol) and 1-[bis(dimethylamino)methylene]-1H-1,2,3-triazolo[4,5-b]pyridinium 3-oxid hexafluorophosphate (HATU, 146 mg, 0.385 mmol) in N,N-dimethylformamide (3 mL) was stirred for 5 hours. The reaction mixture was quenched with brine and saturated NaHCO₃and extracted with ethyl acetate (2×). The combined organic layers were washed with brine, dried over MgSO₄, filtered and concentrated. The concentrate was dissolved in tetrahydrofuran (1.5 mL) and methanol (1.2 mL) and then treated with a solution of lithium hydroxide (11.53 mg, 0.481 mmol) in water (1 mL). The mixture was stirred for 2 hours, diluted with ethyl acetate, and washed with brine. The organic layer was dried over MgSO₄, filtered, and concentrated. The residue was purified by reverse-phase HPLC (see protocol in Example 6) to provide the title compound (49.2 mg, 28%). ¹H NMR (500 MHz, DMSO-d₆) δ ppm 7.48 (t, J=8.9 Hz, 1H), 7.32-7.22 (m, 2H), 7.05 (dd, J=11.4, 2.9 Hz, 1H), 6.86-6.73 (m, 2H), 6.69 (d, J=2.4 Hz, 1H), 6.59 (dd, J=8.5, 2.4 Hz, 1H), 5.07 (d, J=4.4 Hz, 1H), 4.54 (ddd, J=7.4, 2.9, 1.3 Hz, 1H), 4.46 (s, 2H), 4.04 (dt, J=8.8, 3.6 Hz, 1H), 3.39-3.33 (m, 1H), 3.16 (ddd, J=12.4, 7.1, 1.3 Hz, 1H), 2.83 (s, 3H), 2.32-2.17 (m, 1H), 2.12-2.01 (m, 1H), 1.96-1.71 (m, 8H); MS (ESI⁺) m/z 552.1 (M+H)⁺.

### Example 9: N-{3-[2-(4-chloro-3-fluorophenoxy)acetamido]bicyclo[1.1.1]pentan-1-yl}-6,7-difluoro-3,4-dihydro-2H-1,4-benzoxazine-2-carboxamide (Compound 108)

### Example 9A: ethyl 6,7-difluoro-3,4-dihydro-2H-benzo[b][1,4]oxazine-2-carboxylate

To a solution of 2-amino-4,5-difluorophenol (1.0 g, 6.9 mmol) in acetone (14 mL) was added potassium carbonate (2.67 g, 19.3 mmol), followed by ethyl 2,3-dibromopropanoate (1.1 mL, 7.6 mmol). The reaction mixture was refluxed for 16 hours, concentrated, filtered, and concentrated to give the title intermediate (1.5 g, 6.0 mmol, 87% yield) without further purification. MS (ESI⁺) m/z 244 (M+H)⁺.

### Example 9B: 6,7-difluoro-3,4-dihydro-2H-benzo[b][1,4]oxazine-2-carboxylic Acid

To a solution of the product of Example 9A (1.45 g, 5.98 mmol) in a solution of tetrahydrofuran (3 mL):H₂O (3 mL) was added sodium hydroxide (0.039 g, 0.98 mmol). The reaction mixture stirred for 17 hours and then concentrated. The residue was diluted with H₂O (3 mL), acidified with 1 N HCl at 0° C., and extracted with ethyl acetate (3×10 mL). The combined organic layers were dried over anhydrous Na₂SO₄, filtered, and concentrated under reduced pressure to afford the title intermediate (1.0 g, 4.65 mmol, 78% yield). ¹H NMR (501 MHz, DMSO-d₆) δ ppm 6.83 (dd, J=11.7, 7.8 Hz, 1H), 6.55 (dd, J=12.2, 8.2 Hz, 1H), 4.83 (dd, J=4.0, 3.3 Hz, 1H), 3.44-3.34 (m, 2H); MS (ESI+) m/z 216 (M+H)⁺.

### Example 9C: N-{3-[2-(4-chloro-3-fluorophenoxy)acetamido]bicyclo[1.1.1]pentan-1-yl}-6,7-difluoro-3,4-dihydro-2H-1,4-benzoxazine-2-carboxamide

To a mixture of the product of Example 23B (0.10 g, 0.35 mmol) and the product of Example 9B (0.079 g, 0.37 mmol) in N,N-dimethylformamide (2 mL) was added triethylamine (0.2 mL, 1.4 mmol) followed by 1-[bis(dimethylamino)methylene]-1H-1,2,3-triazolo[4,5-b]pyridinium 3-oxid hexafluorophosphate (HATU, 0.15 g, 0.39 mmol). This reaction mixture was allowed to stir at ambient temperature for 24 hours. Then the reaction mixture was partitioned between saturated aqueous NaHCO₃ (20 mL) and ethyl acetate (20 mL). The layers were separated, and the aqueous layer was extracted with ethyl acetate (3×10 mL). The combined organic fractions were dried over anhydrous Na₂SO₄, filtered, and concentrated under reduced pressure. The residue was purified by preparative HPLC [Waters XBridge™ C18 5 μm OBD column, 30×100 mm, flow rate 40 mL/minute, 5-100% gradient of acetonitrile in buffer (0.025 M aqueous ammonium bicarbonate, adjusted to pH 10 with ammonium hydroxide)] to give the title compound (0.015 g, 0.031 mmol, 9% yield). ¹H NMR (501 MHz, DMSO-d₆) δ ppm 8.70 (s, 1H), 8.61 (s, 1H), 7.49 (t, J=8.9 Hz, 1H), 7.07 (dd, J=11.4, 2.9 Hz, 1H), 6.92-6.76 (m, 2H), 6.56 (dd, J=12.2, 8.1 Hz, 1H), 5.98 (s, 1H), 4.47 (s, 2H), 4.43 (dd, J=7.2, 2.9 Hz, 1H), 3.15 (d, J=10.0 Hz, 1H), 2.25 (s, 6H); MS (ESI⁺) m/z 482 (M+H)⁺.

### Example 10: N-{3-[2-(4-chloro-3-fluorophenoxy)acetamido]bicyclo[1.1.1]pentan-1-yl}-3-oxo-3,4-dihydro-2H-1,4-benzoxazine-2-carboxamide (Compound 109)

To a mixture of the product of Example 23B (0.15 g, 0.53 mmol) and 3-oxo-3,4-dihydro-2H-benzo[b][1,4]oxazine-2-carboxylic acid (0.11 g, 0.55 mmol) in N,N-dimethylformamide (3 mL) was added triethylamine (0.3 mL, 2 mmol) followed by 1-[bis(dimethylamino)methylene]-1H-1,2,3-triazolo[4,5-b]pyridinium 3-oxid hexafluorophosphate (HATU, 0.22 g, 0.58 mmol). This reaction mixture was allowed to stir at ambient temperature for 18 hours. Then the reaction mixture was diluted with saturated aqueous NaHCO₃ (20 mL) and ethyl acetate (20 mL). The layers were separated, and the aqueous layer was extracted with ethyl acetate (3×10 mL). The combined organic layers were dried over anhydrous Na₂SO₄, filtered, concentrated under reduced pressure. The residue was purified by preparative HPLC [Waters XBridge™ C18 5 μm OBD column, 30×100 mm, flow rate 40 mL/minute, 5-100% gradient of acetonitrile in buffer (0.025 M aqueous ammonium bicarbonate, adjusted to pH 10 with ammonium hydroxide)] to give the title compound (0.20 g, 0.43 mmol, 82% yield). ¹H NMR (400 MHz, DMSO-d₆) δ ppm 10.84 (s, 1H), 9.04 (s, 1H), 8.71 (s, 1H), 7.49 (t, J=8.8 Hz, 1H), 7.12-7.02 (m, 1H), 6.97-6.83 (m, 5H), 5.00 (s, 1H), 4.46 (s, 2H), 2.23 (s, 6H); MS (ESI⁺) m/z 460 (M+H)⁺.

### Example 11: N-{(3S)-4-[2-(4-chloro-3-fluorophenoxy)acetamido]-3-hydroxybicyclo[2.2.2]octan-1-yl}-3,4-dihydro-2H-1,4-benzoxazine-2-carboxamide (Compound 110)

The procedures described in Example 8C substituting 3,4-dihydro-2H-benzo[b][1,4]oxazine-2-carboxylic acid for Example 8B gave the title compound. ¹H NMR (500 MHz, DMSO-d₆) δ ppm 7.48 (t, J=8.9 Hz, 1H), 7.26 (s, 1H), 7.17 (s, 1H), 7.05 (dd, J=11.4, 2.9 Hz, 1H), 6.83 (dd, J=8.9, 2.7 Hz, 1H), 6.76 (dd, J=8.2, 1.4 Hz, 1H), 6.69 (td, J=7.6, 1.5 Hz, 1H), 6.58 (dd, J=7.9, 1.6 Hz, 1H), 6.51 (td, J=7.6, 1.6 Hz, 1H), 4.46 (s, 2H), 4.39-4.32 (m, 1H), 4.04 (dd, J=9.6, 3.1 Hz, 1H), 3.37 (dd, J=11.9, 2.9 Hz, 1H), 3.19-3.10 (m, 1H), 2.27 (dddd, J=12.1, 9.3, 5.0, 2.4 Hz, 1H), 2.06 (ddd, J=12.3, 10.1, 5.6 Hz, 1H), 1.96-1.67 (m, 8H); MS (ESI⁺) m/z 504.1 (M+H)⁺.

### Example 12: N-{(3S)-4-[2-(4-chloro-3-fluorophenoxy)acetamido]-3-hydroxybicyclo[2.2.2]octan-1-yl}-4-methyl-3,4-dihydro-2H-1,4-benzoxazine-2-carboxamide (Compound 111)

The procedures described in Example 8C substituting 4-methyl-3,4-dihydro-2H-benzo[b][1,4]oxazine-2-carboxylic acid for Example 8B gave the title compound. ¹H NMR (400 MHz, DMSO-d₆) δ ppm 7.48 (t, J=8.9 Hz, 1H), 7.24 (d, J=14.7 Hz, 2H), 7.05 (dd, J=11.4, 2.8 Hz, 1H), 6.85-6.76 (m, 3H), 6.70 (dd, J=8.4, 1.6 Hz, 1H), 6.61 (td, J=7.5, 1.6 Hz, 1H), 4.55 (dd, J=7.7, 2.7 Hz, 1H), 4.47 (s, 2H), 4.04 (dd, J=9.6, 3.1 Hz, 1H), 3.33 (dd, J=11.8, 2.9 Hz, 1H), 3.11 (dd, J=11.8, 7.6 Hz, 1H), 2.81 (s, 3H), 2.27 (ddt, J=12.4, 9.4, 2.9 Hz, 1H), 2.07 (td, J=11.7, 5.0 Hz, 1H), 1.97-1.69 (m, 8H); MS (ESI⁺) m/z 518.3 (M+H)⁺.

### Example 13: 6-chloro-N-{3-[2-(4-chloro-3-fluorophenoxy)acetamido]bicyclo[1.1.1]pentan-1-yl}-4-methyl-3,4-dihydro-2H-1,4-benzoxazine-2-carboxamide (Compound 112)

### Example 13A: ethyl 6-chloro-3,4-dihydro-2H-benzo[b][1,4]oxazine-2-carboxylate

To a solution of 2-amino-4-chlorophenol (2.00 g, 13.9 mmol) in acetone (77 mL) was added potassium carbonate (5.39 g, 39.0 mmol) followed by ethyl 2,3-dibromopropanoate (2.2 mL, 15 mmol). The reaction mixture was refluxed for 16 hours providing the title intermediate without isolation. MS (ESI⁺) m/z 283 (M+CH₃CN)⁺.

### Example 13B: ethyl 6-chloro-4-methyl-3,4-dihydro-2H-benzo[b][1,4]oxazine-2-carboxylate

To the reaction mixture of Example 13A was added iodomethane (3 mL, 49 mmol) and additional potassium carbonate (1.00 g, 6.97 mmol). The reaction mixture was refluxed for 5 hours and then stirred at ambient temperature for 4 days, resulting in incomplete conversion. The reaction mixture was filtered, concentrated, and purified by preparative HPLC [Waters XBridge™ C18 5 μm OBD column, 50×100 mm, flow rate 90 mL/minute, 5-100% gradient of acetonitrile in buffer (0.025 M aqueous ammonium bicarbonate, adjusted to pH 10 with ammonium hydroxide)] to give the title intermediate (1 g, 4 mmol, 28% yield). ¹H NMR (501 MHz, DMSO-d₆) δ ppm 6.77 (d, J=8.5 Hz, 1H), 6.69 (d, J=2.4 Hz, 1H), 6.62 (dd, J=8.5, 2.4 Hz, 1H), 5.06 (dd, J=4.0, 3.2 Hz, 1H), 4.25-4.06 (m, 2H), 3.52-3.36 (m, 2H), 2.82 (s, 3H), 1.17 (t, J=7.1 Hz, 3H); MS (ESI⁺) m/z 256 (M+H)⁺.

### Example 13C: 6-chloro-4-methyl-3,4-dihydro-2H-benzo[b][1,4]oxazine-2-carboxylic Acid

To a solution of the product of Example 13B (0.10 g, 0.39 mmol) in a solution of tetrahydrofuran (1 mL):H₂O (1 mL) was added sodium hydroxide (0.039 g, 0.98 mmol). The reaction mixture stirred for 3 days and then was concentrated. The residue was diluted with H₂O (3 mL), acidified with 1 N HCl at 0° C., and extracted with ethyl acetate (3×10 mL). The combined organic layers were dried over anhydrous Na₂SO₄, filtered, and concentrated under reduced pressure to afford the title intermediate (0.078 g, 0.34 mmol, 88% yield). ¹H NMR (400 MHz, DMSO-d₆) δ ppm 6.73 (d, J=8.4 Hz, 1H), 6.67 (d, J=2.4 Hz, 1H), 6.60 (dd, J=8.4, 2.5 Hz, 1H), 4.85 (s, 1H), 2.81 (s, 3H), 1.24 (s, 2H); MS (ESI⁻) m/z 226 (M−H)⁺.

### Example 13D: 6-chloro-N-{3-[2-(4-chloro-3-fluorophenoxy)acetamido]bicyclo[1.1.1]pentan-1-yl}-4-methyl-3,4-dihydro-2H-1,4-benzoxazine-2-carboxamide

To a mixture of the product of Example 23B (0.030 g, 0.11 mmol) and the product of Example 13C (0.025 g, 0.11 mmol) in N,N-dimethylformamide (0.6 mL) was added triethylamine (0.06 mL, 0.4 mmol) followed by 1-[bis(dimethylamino)methylene]-1H-1,2,3-triazolo[4,5-b]pyridinium 3-oxid hexafluorophosphate (HATU, 0.044 g, 0.12 mmol). This reaction mixture was allowed to stir at ambient temperature for 5.5 hours. Then the reaction mixture was diluted with saturated aqueous NaHCO₃ (20 mL) and ethyl acetate (20 mL). The layers were separated, and the aqueous layer was extracted with ethyl acetate (3×10 mL). The combined organic layers were dried over anhydrous Na₂SO₄, filtered, and concentrated under reduced pressure. The residue was purified by preparative HPLC [Waters XBridge™ C18 5 μm OBD column, 30×100 mm, flow rate 40 mL/minute, 5-100% gradient of acetonitrile in buffer (0.025 M aqueous ammonium bicarbonate, adjusted to pH 10 with ammonium hydroxide)] to give the title compound (0.035 g, 0.071 mmol, 67% yield). ¹H NMR (400 MHz, DMSO-d₆) δ ppm 8.71 (s, 1H), 8.67 (s, 1H), 7.49 (t, J=8.9 Hz, 1H), 7.07 (dd, J=11.3, 2.9 Hz, 1H), 6.85 (ddd, J=9.0, 2.9, 1.2 Hz, 1H), 6.80 (d, J=8.4 Hz, 1H), 6.70 (d, J=2.4 Hz, 1H), 6.62 (dd, J=8.5, 2.4 Hz, 1H), 4.61 (dd, J=7.3, 3.0 Hz, 1H), 4.47 (s, 2H), 3.40-3.37 (m, 1H), 3.18 (dd, J=12.0, 7.4 Hz, 1H), 2.83 (s, 3H), 2.26 (s, 6H); MS (ESI⁺) m/z 494 (M+H)⁺.

### Example 14: 6-chloro-N-{3-[2-(4-chloro-3-fluorophenoxy)acetamido]bicyclo[1.1.1]pentan-1-yl}-3,4-dihydro-2H-1,4-benzoxazine-2-carboxamide (Compound 113)

To a mixture of Example 23B (0.070 g, 0.25 mmol) and 6-chloro-3,4-dihydro-2H-benzo[b][1,4]oxazine-2-carboxylic acid (0.055 g, 0.26 mmol) in N,N-dimethylformamide (1.4 mL) was added triethylamine (0.14 mL, 0.98 mmol) followed by 1-[bis(dimethylamino)methylene]-1H-1,2,3-triazolo[4,5-b]pyridinium 3-oxid hexafluorophosphate (HATU, 0.10 g, 0.27 mmol). This reaction mixture was allowed to stir at ambient temperature for 3 hours. Then the reaction mixture was partitioned between saturated aqueous NaHCO₃ (20 mL) and ethyl acetate (20 mL). The layers were separated, and the aqueous layer was extracted with ethyl acetate (3×10 mL). The combined organic layers were dried over anhydrous Na₂SO₄, filtered, and concentrated under reduced pressure. The residue was purified by preparative HPLC [Waters XBridge™ C18 5 μm OBD column, 30×100 mm, flow rate 40 mL/minute, 5-100% gradient of acetonitrile in buffer (0.025 M aqueous ammonium bicarbonate, adjusted to pH 10 with ammonium hydroxide)] to give the title compound (0.075 g, 0.16 mmol, 64% yield). ¹H NMR (400 MHz, DMSO-d₆) δ ppm 8.71 (s, 1H), 8.63 (s, 1H), 7.49 (t, J=8.9 Hz, 1H), 7.07 (dd, J=11.3, 2.8 Hz, 1H), 6.92-6.82 (m, 1H), 6.77 (d, J=8.5 Hz, 1H), 6.60 (d, J=2.5 Hz, 1H), 6.51 (dd, J=8.5, 2.5 Hz, 1H), 6.15 (s, 1H), 4.47 (s, 2H), 4.42 (dd, J=7.4, 2.9 Hz, 1H), 3.23-3.10 (m, 2H), 2.26 (s, 6H); MS (ESI⁺) m/z 480 (M+H)⁺.

### Example 15: 6-chloro-N-{(3S)-4-[2-(4-chloro-3-fluorophenoxy)acetamido]-3-hydroxybicyclo[2.2.2]octan-1-yl}-3,4-dihydro-2H-1,4-benzoxazine-2-carboxamide (Compound 114)

The methodologies described in Example 8C substituting 6-chloro-3,4-dihydro-2H-benzo[b][1,4]oxazine-2-carboxylic acid for Example 8B gave the title compound. ¹H NMR (500 MHz, methanol-d₄) δ ppm 7.40-7.33 (m, 1H), 6.92 (dd, J=10.9, 2.9 Hz, 1H), 6.84-6.75 (m, 2H), 6.61 (d, J=2.4 Hz, 1H), 6.53 (dd, J=8.6, 2.5 Hz, 1H), 4.45 (d, J=1.5 Hz, 2H), 4.41 (dt, J=7.2, 3.0 Hz, 1H), 4.31-4.19 (m, 1H), 3.50 (dd, J=12.0, 2.8 Hz, 1H), 3.26 (dd, J=12.0, 7.4 Hz, 1H), 2.54-2.40 (m, 1H), 2.20-1.80 (m, 9H); MS (ESI⁺) m/z 538.3 (M+H)⁺.

### Example 16: (2R)-6-chloro-N-{(3S)-4-[2-(4-chloro-3-fluorophenoxy)acetamido]-3-hydroxybicyclo[2.2.2]octan-1-yl}-2,3-dihydro-1,4-benzodioxine-2-carboxamide (Compound 115)

The methodologies described in Example 6 substituting (R)-6-chloro-2,3-dihydrobenzo[b][1,4]dioxine-2-carboxylic acid for (S)-6-fluorochroman-2-carboxylic acid gave the title compound. ¹H NMR (400 MHz, DMSO-d₆) δ ppm 7.52-7.45 (m, 2H), 7.26 (s, 1H), 7.05 (dd, J=11.4, 2.8 Hz, 1H), 7.01-6.94 (m, 2H), 6.90 (dd, J=8.7, 2.5 Hz, 1H), 6.82 (ddd, J=9.0, 2.9, 1.2 Hz, 1H), 5.08 (d, J=4.3 Hz, 1H), 4.66 (dd, J=6.1, 2.7 Hz, 1H), 4.46 (s, 2H), 4.33 (dd, J=11.6, 2.7 Hz, 1H), 4.15 (dd, J=11.6, 6.1 Hz, 1H), 4.04 (dt, J=8.6, 3.6 Hz, 1H), 2.25 (ddd, J=12.4, 9.4, 2.4 Hz, 1H), 2.06 (ddd, J=12.2, 10.1, 5.1 Hz, 1H), 1.97-1.66 (m, 8H); MS (ESI⁺) m/z 539.2 (M+H)⁺.

### Example 17: (2R)-6-chloro-N-{(3S)-4-[2-(3,4-dichlorophenoxy)acetamido]-3-hydroxybicyclo[2.2.2]octan-1-yl}-2,3-dihydro-1,4-benzodioxine-2-carboxamide (Compound 116)

### Example 17A: N-[(2S)-4-amino-2-hydroxybicyclo[2.2.2]octan-1-yl]-2-(3,4-dichlorophenoxy)acetamide

The title compound was synthesized using the methodologies described in Examples 2A-J substituting 2-(3,4-dichlorophenoxy)acetic acid for 2-(4-chloro-3-fluorophenoxy)acetic acid in Example 2G. MS (ESI⁺) m/z 359.0 (M+H)⁺.

### Example 17B: (2R)-6-chloro-N-{(3S)-4-[2-(3,4-dichlorophenoxy)acetamido]-3-hydroxybicyclo[2.2.2]octan-1-yl}-2,3-dihydro-1,4-benzodioxine-2-carboxamide

The methodologies described in Example 6 substituting Example 17A and (R)-6-chloro-2,3-dihydrobenzo[b][1,4]dioxine-2-carboxylic acid for Example 2J and (S)-6-fluorochroman-2-carboxylic acid, respectively, gave the title compound. ¹H NMR (400 MHz, DMSO-d₆) δ ppm 7.53 (d, J=8.9 Hz, 2H), 7.28-7.20 (m, 2H), 7.01-6.92 (m, 3H), 6.90 (dd, J=8.6, 2.5 Hz, 1H), 5.08 (d, J=4.4 Hz, 1H), 4.66 (dd, J=6.1, 2.7 Hz, 1H), 4.47 (s, 2H), 4.33 (dd, J=11.6, 2.6 Hz, 1H), 4.15 (dd, J=11.6, 6.1 Hz, 1H), 4.03 (dt, J=8.7, 3.7 Hz, 1H), 2.25 (ddd, J=12.5, 9.5, 2.3 Hz, 1H), 2.06 (ddd, J=12.3, 10.2, 5.0 Hz, 1H), 1.94-1.73 (m, 8H); MS (ESI⁺) m/z 555.2 (M+H)⁺.

### Example 18: 6,7-dichloro-N-{3-[2-(3,4-difluorophenoxy)acetamido]bicyclo[1.1.1]pentan-1-yl}-2,3-dihydro-1,4-benzodioxine-2-carboxamide (Compound 117)

### Example 18A: ethyl 6,7-dichloro-2,3-dihydrobenzo[b][1,4]dioxine-2-carboxylate

A mixture of 4,5-dichlorobenzene-1,2-diol (0.806 g, 4.5 mmol) was refluxed with ethyl 2,3-dibromopropanoate (1.170 g, 4.50 mmol) in acetone (10 mL) with potassium carbonate (0.933 g, 6.75 mmol) for 7 hours. The reaction mixture was concentrated and extracted with ethyl acetate (100 mL). The ethyl acetate layer was washed with water (30 mL×2), dried over Na₂SO₄, and concentrated. The residue was purified by flash column chromatography on silica gel (40 g) eluted with heptane and ethyl acetate (5 to 20%) to give the title intermediate (0.93 g, 75% yield). ¹H NMR (400 MHz, DMSO-d₆) δ ppm 7.30 (s, 1H), 7.21 (s, 1H), 5.28 (t, J=3 Hz, 1H), 4.51 (dd, J=12, 3 Hz, 1H), 4.33 (dd, J=12, 3 Hz, 1H), 4.17 (q, J=7 Hz, 2H), 1.18 (t, J=7 Hz, 3H); MS (ESI⁺) m/z 277 (M+H)⁺.

### Example 18B: 6,7-dichloro-2,3-dihydrobenzo[b][1,4]dioxine-2-carboxylic acid

To Example 18A (910 mg, 3.28 mmol) in CH₃OH (20 mL) was added 4 N sodium hydroxide (8210 μL, 32.8 mmol) solution. The mixture was stirred at room temperature for 2 hours. Then the mixture was concentrated, and acidified with 1 N aqueous HCl solution to pH-6. The resulting solid was collected by filtration, and dried to give the title intermediate as a solid (755 mg, 91% yield)¹H NMR (400 MHz, DMSO-d₆) δ ppm 7.28 (s, 1H), 7.21 (s, 1H), 5.17 (t, J=3 Hz, 1H), 4.51 (dd, J=12, 3 Hz, 1H), 4.33 (dd, J=12, 3 Hz, 1H); MS (ESI−) m/z 247 (M−H)⁻.

### Example 18C: tert-butyl (3-(6,7-dichloro-2,3-dihydrobenzo[b][1,4]dioxine-2-carboxamido)bicyclo[1.1.1]pentan-1-yl)carbamate

To a mixture of Example 18B (249 mg, 1), 1-[bis(dimethylamino)methylene]-1H-1,2,3-triazolo[4,5-b]pyridinium 3-oxid hexafluorophosphate (HATU, 418 mg, 1.100 mmol) and tert-butyl (3-aminobicyclo[1.1.1]pentan-1-yl)carbamate (198 mg, 1.000 mmol) was added N-ethyl-N-isopropylpropan-2-amine (258 mg, 2.000 mmol) in N,N-dimethylformamide (4 mL). The mixture was stirred at room temperature for 20 minutes, and then water (20 mL) was added. The mixture was extracted with ethyl acetate (100 mL). The organic phase w as washed with water (30 mL) and brine (30 mL), dried over Na₂SO₄, and concentrated to give the title intermediate as a solid (0.43 g, 100% yield) which was used without further purification. ¹H NMR (400 MHz, DMSO-d₆) δ ppm 8.80 (s, 1H), 7.96 (s, 1H), 7.23 (s, 1H), 7.22 (s, 1H), 4.80 (dd, J=6, 3 Hz, 1H), 4.30 (m, 2H), 2.15 (s, 6H), 1.38 (s, 9H); MS (ESI⁺) m/z 429 (M+H)⁺.

### Example 18D: N-(3-aminobicyclo[1.1.1]pentan-1-yl)-6,7-dichloro-2,3-dihydrobenzo[b][1,4]dioxine-2-carboxamide, trifluoroacetic acid

To tert-butyl (3-(6,7-dichloro-2,3-dihydrobenzo[b][1,4]dioxine-2-carboxamido)bicyclo[1.1.1]pentan-1-yl)carbamate (425 mg, 0.990 mmol) in dichloromethane (12 mL) was added trifluoroacetic acid (3 mL). The mixture was stirred at room temperature for 2 hours. The mixture was concentrated, and the residue was directly purified by preparative HPLC [Waters XBridge™ C18 5 μm OBD column, 50×100 mm, flow rate 90 mL/minute, 5-100% gradient of acetonitrile in buffer (0.1% trifluoroacetic acid)] to give the title intermediate (295 mg, 67%). ¹H NMR (400 MHz, DMSO-d₆) δ ppm 8.98 (s, 1H), 8.72 (s, 3H), 7.23 (s, 1H), 7.22 (s, 1H), 4.87 (dd, J=5, 4 Hz, 1H), 4.32 (m, 2H), 2.24 (s, 6H); MS (ESI⁺) m/z 329 (M+H)⁺.

### Example 18E: 6,7-dichloro-N-{3-[2-(3,4-difluorophenoxy)acetamido]bicyclo[1.1.1]pentan-1-yl}-2,3-dihydro-1,4-benzodioxine-2-carboxamide

To a mixture of Example 18D (31.0 mg, 0.070 mmol), 1-[bis(dimethylamino)methylene]-1H-1,2,3-triazolo[4,5-b]pyridinium 3-oxid hexafluorophosphate (HATU, 26.6 mg, 0.070 mmol) and 2-(3,4-difluorophenoxy)acetic acid (13.17 mg, 0.07 mmol) was added N-ethyl-N-isopropylpropan-2-amine (45.2 mg, 0.350 mmol) in N,N-dimethylformamide (1 mL). The mixture was stirred at room temperature for 20 minutes, then water (0.02 mL) was added, and the mixture was directly purified by preparative HPLC [Waters XBridge™ C18 5 μm OBD column, 50×100 mm, flow rate 90 mL/minute, 5-100% gradient of acetonitrile in buffer (0.1% trifluoroacetic acid)] to give the title intermediate (25 mg, 72%). ¹H NMR (400 MHz, DMSO-d₆) δ ppm 8.83 (s, 1H), 8.69 (s, 1H), 7.36 (q, J=8 Hz, 1H), 7.23 (s, 1H), 7.21 (s, 1H), 7.08 (m, 1H), 6.80 (m, 1H), 4.82 (dd, J=5, 3 Hz, 1H), 4.43 (s, 2H), 4.29 (m, 2H), 2.25 (s, 6H); MS (ESI−) m/z 499 (M−H)⁻.

### Example 19: N-{3-[2-(4-chloro-3-fluorophenoxy)acetamido]bicyclo[1.1.1]pentan-1-yl}-6,7-difluoro-2,3-dihydro-1,4-benzodioxine-2-carboxamide (Compound 118)

### Example 19A: ethyl 6,7-difluoro-2,3-dihydrobenzo[b][1,4]dioxine-2-carboxylate

The reaction and purification conditions described in Example 18A substituting 4,5-difluorobenzene-1,2-diol for 4,5-dichlorobenzene-1,2-diol gave the title compound (140 mg, 21% yield). ¹H NMR (400 MHz, DMSO-d₆) δ ppm 7.16 (dd, J=8, 11 Hz, 1H), 7.06 (dd, J=8, 11 Hz, 1H), 5.22 (t, J=3 Hz, 1H), 4.47 (dd, J=12, 3 Hz, 1H), 4.30 (dd, J=12, 3 Hz, 1H), 4.17 (q, J=7 Hz, 2H), 1.19 (t, J=7 Hz, 3H); MS (DCI+) m/z 262 (M+NH₄)⁺.

### Example 19B: 6,7-difluoro-2,3-dihydrobenzo[b][1,4]dioxine-2-carboxylic acid

The reaction and purification conditions described in Example 18B substituting Example 19A for Example 18A gave the title compound (125 mg, 100% yield). ¹H NMR (400 MHz, DMSO-d₆) δ ppm 6.95 (dd, J=8, 11 Hz, 1H), 6.87 (dd, J=8, 11 Hz, 1H), 4.25 (m, 2H), 4.09 (dd, J=7, 11 Hz, 1H); MS (ESI−) m/z 215 (M−H)⁻.

### Example 19C: N-{3-[2-(4-chloro-3-fluorophenoxy)acetamido]bicyclo[1.1.1]pentan-1-yl}-6,7-difluoro-2,3-dihydro-1,4-benzodioxine-2-carboxamide

The reaction and purification conditions described in Example 23C substituting Example 19B for 2,3-dihydrobenzo[b][1,4]dioxine-2-carboxylic acid gave the title compound (120 mg, 59% yield). ¹H NMR (400 MHz, DMSO-d₆) δ ppm 8.79 (s, 1H), 8.69 (s, 1H), 7.47 (t, J=8, 1H), 7.04 (m, 3H), 6.82 (br d, J=8 Hz, 1H), 4.72 (dd, J=6, 3 Hz, 1H), 4.44 (s, 2H), 4.29 (dd, J=11, 3 Hz, 1H), 4.17 (dd, J=11, 6 Hz, 1H), 2.25 (s, 6H); MS (ESI⁺) m/z 483 (M+H)⁺.

### Example 20: (2R)-6-chloro-N-{3-[2-(4-chloro-3-fluorophenoxy)acetamido]bicyclo[1.1.1]pentan-1-yl}-2,3-dihydro-1,4-benzodioxine-2-carboxamide (Compound 119)

The reaction and purification conditions described in Example 23C substituting (R)-6-chloro-2,3-dihydrobenzo[b][1,4]dioxine-2-carboxylic acid for 2,3-dihydrobenzo[b][1,4]dioxine-2-carboxylic acid gave the title compound (43 mg, 81% yield). ¹H NMR (400 MHz, DMSO-d₆) δ ppm 8.82 (s, 1H), 8.71 (s, 1H), 7.49 (t, J=8, 1H), 7.07 (dd, J=9, 3, 1H), 6.98 (m, 2H), 6.93 (dd, J=8, 3 Hz, 1H), 6.85 (br d, J=8 Hz, 1H), 4.74 (dd, J=6, 3 Hz, 1H), 4.47 (s, 2H), 4.35 (dd, J=11, 3 Hz, 1H), 4.20 (dd, J=11, 6 Hz, 1H), 2.25 (s, 6H); MS (ESI⁺) m/z 481 (M+H)⁺.

### Example 21: (2R)—N-{3-[2-(4-chloro-3-fluorophenoxy)acetamido]bicyclo[1.1.1]pentan-1-yl}-6-fluoro-3,4-dihydro-2H-1-benzopyran-2-carboxamide (Compound 120)

The reaction and purification conditions described in Example 23C substituting (R)-6-fluoro-2,3-dihydrobenzo[b][1,4]dioxine-2-carboxylic acid for 2,3-dihydrobenzo[b][1,4]dioxine-2-carboxylic acid gave the title compound (44 mg, 86% yield). ¹H NMR (400 MHz, DMSO-d₆) δ ppm 8.73 (s, 1H), 8.61 (s, 1H), 7.50 (t, J=8 Hz, 1H), 7.06 (dd, J=9, 3 Hz, 1H), 6.85-6.95 (m, 4H), 4.48 (s, 2H), 4.43 (dd, J=9, 3 Hz, 1H), 2.80 (m, 1H), 2.68 (m, 1H), 2.27 (s, 6H), 2.13 (m, 1H), 1.82 (m, 1H); MS (ESI⁺) m/z 463 (M+H)⁺.

### Example 22: (2S)—N-{3-[2-(4-chloro-3-fluorophenoxy)acetamido]bicyclo[1.1.1]pentan-1-yl}-6-fluoro-3,4-dihydro-2H-1-benzopyran-2-carboxamide (Compound 121)

The reaction and purification conditions described in Example 23C substituting (S)-6-fluoro-2,3-dihydrobenzo[b][1,4]dioxine-2-carboxylic acid for 2,3-dihydrobenzo[b][1,4]dioxine-2-carboxylic acid gave the title compound (42 mg, 91% yield). ¹H NMR (400 MHz, DMSO-d₆) δ ppm 8.73 (s, 1H), 8.61 (s, 1H), 7.50 (t, J=8 Hz, 1H), 7.06 (dd, J=9, 3 Hz, 1H), 6.85-6.95 (m, 4H), 4.48 (s, 2H), 4.43 (dd, J=9, 3 Hz, 1H), 2.80 (m, 1H), 2.68 (m, 1H), 2.27 (s, 6H), 2.13 (m, 1H), 1.82 (m, 1H); MS (ESI⁺) m/z 463 (M+H)⁺.

### Example 23: N-{3-[2-(4-chloro-3-fluorophenoxy)acetamido]bicyclo[1.1.1]pentan-1-yl}-2,3-dihydro-1,4-benzodioxine-2-carboxamide (Compound 122)

### Example 23A: tert-butyl (3-(2-(4-chloro-3-fluorophenoxy)acetamido)bicyclo[1.1.1]pentan-1-yl)carbamate

To a solution of 2-(4-chloro-3-fluorophenoxy)acetic acid (Aldlab Chemicals, 2.01 g, 9.84 mmol) in N,N-dimethylformamide (25 mL) was added N-ethyl-N-isopropylpropan-2-amine (3.96 mL, 22.7 mmol) followed by 2-(3H-[1,2,3]triazolo[4,5-b]pyridin-3-yl)-1,1,3,3-tetramethylisouronium hexafluorophosphate (V) (3.02 g, 7.94 mmol). This mixture was stirred at ambient temperature for 5 minutes, and then tert-butyl (3-aminobicyclo[1.1.1]pentan-1-yl)carbamate (PharmaBlock, 1.5 g, 7.57 mmol) was added. The mixture was allowed to stir at ambient temperature for 16 hours. The reaction mixture was quenched with saturated, aqueous NH₄Cl (20 mL) and then washed with CH₂Cl₂ (25 mL). The aqueous layer was extracted with CH₂Cl₂ (3×5 mL), and the combined organic fractions were dried over anhydrous Na₂SO₄, filtered, and concentrated under reduced pressure. The residue was purified by column chromatography (SiO₂, 10% ethyl acetate/heptanes to 80% ethyl acetate/heptanes) to give the title compound (2.65 g, 6.89 mmol, 91% yield). MS (ESI⁺) m/z 402 (M+NH₄)⁺.

### Example 23B: N-(3-aminobicyclo[1.1.1]pentan-1-yl)-2-(4-chloro-3-fluorophenoxy)acetamide

To solution of Example 23A (9 g, 23.39 mmol) in dichloromethane (100 mL) was added trifluoroacetic acid (30 mL, 389 mmol) at 0° C. The mixture was stirred at ambient temperature for 12 hours. The mixture was concentrated under reduced pressure, and the residue was diluted with water (300 mL). The aqueous phase was adjusted to pH=8 with NaHCO₃and then extracted with dichloromethane (4×150 mL). The combined organic layer was dried (Na₂SO₄) and concentrated under reduced pressure to provide 6 g (90%) of the title compound. MS (APCI) m/z 285 (M+H)⁺.

### Example 23C: N-{3-[2-(4-chloro-3-fluorophenoxy)acetamido]bicyclo[1.1.1]pentan-1-yl}-2,3-dihydro-1,4-benzodioxine-2-carboxamide

To a solution of Example 23B (40 mg, 0.140 mmol) in N,N-dimethylformamide (0.8 mL) were added 2,3-dihydrobenzo[b][1,4]dioxine-2-carboxylic acid (27.8 mg, 0.155 mmol), 1-[bis(dimethylamino)methylene]-1H-1,2,3-triazolo[4,5-b]pyridinium 3-oxid hexafluorophosphate (HATU, 58.8 mg, 0.155 mmol), and N,N-diisopropylethylamine (0.074 mL, 0.421 mmol) at room temperature. The reaction mixture was stirred for 1 hour at room temperature. The mixture was purified by preparative HPLC [Waters XBridge™ C18 5 μm OBD™ column, 30×100 mm, flow rate 40 mL/minute, 5-100% gradient of acetonitrile in buffer (0.1% trifluoroacetic acid in water)] to give the title compound. (45 mg, 0.101 mmol, 71.7% yield). ¹H NMR (501 MHz, DMSO-d₆) δ ppm 8.78 (s, 1H), 8.70 (s, 1H), 7.47 (t, J=8.9 Hz, 1H), 7.05 (dd, J=11.3, 2.9 Hz, 1H), 6.97-6.92 (m, 1H), 6.87-6.80 (m, 4H), 4.67 (dd, J=6.5, 2.7 Hz, 1H), 4.45 (s, 2H), 4.31 (dd, J=11.6, 2.7 Hz, 1H), 4.12 (dd, J=11.6, 6.5 Hz, 1H), 2.24 (s, 6H); MS (ESI⁺) m/z 464 (M+NH₄).

### Example 24: N-{(2R)-4-[2-(4-chloro-3-fluorophenoxy)acetamido]-2-hydroxybicyclo[2.2.2]octan-1-yl}-2,3-dihydro-1,4-benzodioxine-2-carboxamide (Compound 123)

### Example 24A: (R)—N-(4-amino-3-hydroxybicyclo[2.2.2]octan-1-yl)-2-(4-chloro-3-fluorophenoxy)acetamide 2,2,2-trifluoroacetate

The title compound was isolated by chiral preparative SFC (supercritical fluid chromatography) of Example 30E as the first peak eluted off the column, followed by reverse phase HPLC purification to give the title compound as a trifluoroacetic acid salt. The preparative SFC (supercritical fluid chromatography) was performed on a Thar 200 preparative SFC (SFC-5) system using a Chiralpak® IC, 300×50 mm I.D., 10 μm column. The column was heated at 38° C., and the backpressure regulator was set to maintain 100 bar. The mobile phase A is CO₂ and B is isopropanol (0.1% ammonium hydroxide). The chromatography was performed isocratically at 45% of mobile phase B at a flow rate of 200 mL/minute. Fraction collection was time triggered with UV monitor wavelength set at 220 nm. Preparative HPLC was performed on a Gilson 281 semi-preparative HPLC system using a Phenomenex® Luna® C18(2) 10 μm 100 Å AXIA™ column (250 mm×80 mm) column. A gradient of acetonitrile (A) and 0.075% trifluoroacetic acid in water (B) was used, at a flow rate of 80 mL/minute. A linear gradient was used from about 30% of A to about 100% of A over about 30 minutes. Detection method was UV at wavelength of 220 nM and 254 nM. ¹H NMR (400 MHz, methanol-d₄) δ ppm 7.36 (t, J=8.77 Hz, 1H), 6.89 (dd, J=10.74, 2.85 Hz, 1H), 6.79 (br d, J=9.21 Hz, 1H), 4.43 (s, 2H), 3.94 (br d, J=8.33 Hz, 1H), 2.55 (br t, J=12.50 Hz, 1H), 2.35-1.84 (m, 8H), 1.83-1.58 (m, 2H); MS (ESI⁺) m/z 343.0 (M+H)⁺.

### Example 24B: N-{(2R)-4-[2-(4-chloro-3-fluorophenoxy)acetamido]-2-hydroxybicyclo[2.2.2]octan-1-yl}-2,3-dihydro-1,4-benzodioxine-2-carboxamide

To a solution of Example 24A (64 mg, 0.140 mmol) in N,N-dimethylformamide (0.8 mL) were added 2,3-dihydrobenzo[b][1,4]dioxine-2-carboxylic acid (27.8 mg, 0.154 mmol), 1-[bis(dimethylamino)methylene]-1H-1,2,3-triazolo[4,5-b]pyridinium 3-oxid hexafluorophosphate (HATU, 58.6 mg, 0.154 mmol), and N.N-diisopropylethylamine (0.073 mL, 0.420 mmol) at room temperature. The reaction mixture was stirred 1 hour at room temperature. The mixture was purified by preparative HPLC [Waters XBridge™ C18 5 μm OBD™ column, 30×100 mm, flow rate 40 mL/minute, 5-100% gradient of acetonitrile in buffer (0.1% trifluoroacetic acid in water)] to give the title compound. (25 mg, 0.05 mmol, 35.3% yield). ¹H NMR (400 MHz, DMSO-d₆) δ ppm 7.47 (s, 1H), 7.44 (t, J=8.9 Hz, 1H), 7.21 (d, J=7.6 Hz, 1H), 6.98 (dd, J=11.4, 2.9 Hz, 1H), 6.94-6.88 (m, 1H), 6.86-6.79 (m, 3H), 6.77 (ddd, J=9.0, 2.9, 1.2 Hz, 1H), 4.65 (dt, J=6.5, 3.1 Hz, 1H), 4.39 (s, 2H), 4.29 (dt, J=11.5, 3.0 Hz, 1H), 4.09 (ddd, J=11.6, 6.6, 3.3 Hz, 1H), 3.92 (ddd, J=39.3, 9.5, 3.2 Hz, 1H), 2.24 (tdd, J=12.3, 9.5, 2.5 Hz, 1H), 2.13 (ddt, J=17.7, 7.6, 3.0 Hz, 1H), 1.95-1.64 (m, 8H); MS (ESI⁺) m/z 522 (M+H)⁺.

### Example 25: (2R)—N-{3-[2-(4-chloro-3-fluorophenoxy)acetamido]bicyclo[1.1.1]pentan-1-yl}-2,3-dihydro-1,4-benzodioxine-2-carboxamide (Compound 124)

The reaction and purification conditions described in Example 23C substituting (R)-2,3-dihydrobenzo[b][1,4]dioxine-2-carboxylic acid for 2,3-dihydrobenzo[b][1,4]dioxine-2-carboxylic acid gave the title compound (41 mg, 90% yield). ¹H NMR (400 MHz, DMSO-d₆) δ ppm 8.79 (s, 1H), 8.71 (s, 1H), 7.49 (t, J=8 Hz, 1H), 7.07 (dd, J=9, 3 Hz, 1H), 6.95 (m, 1H), 6.85 (m, 4H), 4.69 (dd, J=6, 3 Hz, 1H), 4.47 (s, 2H), 4.32 (dd, J=11, 3 Hz, 1H), 4.13 (dd, J=11, 6 Hz, 1H), 2.25 (s, 6H); MS (ESI⁺) m/z 447 (M+H)⁺.

### Example 26: (2S)—N-{3-[2-(4-chloro-3-fluorophenoxy)acetamido]bicyclo[1.1.1]pentan-1-yl}-2,3-dihydro-1,4-benzodioxine-2-carboxamide (Compound 125)

The reaction and purification conditions described in Example 23C substituting (S)-2,3-dihydrobenzo[b][1,4]dioxine-2-carboxylic acid for 2,3-dihydrobenzo[b][1,4]dioxine-2-carboxylic acid gave the title compound (42 mg, 91% yield). ¹H NMR (400 MHz, DMSO-d₆) δ ppm 8.79 (s, 1H), 8.71 (s, 1H), 7.49 (t, J=8 Hz, 1H), 7.07 (dd, J=9, 3 Hz, 1H), 6.95 (m, 1H), 6.85 (m, 4H), 4.69 (dd, J=6, 3 Hz, 1H), 4.47 (s, 2H), 4.32 (dd, J=11, 3 Hz, 1H), 4.13 (dd, J=11, 6 Hz, 1H), 2.25 (s, 6H); MS (ESI⁺) m/z 447 (M+H)⁺.

### Example 27: (2R)—N-{(2S)-4-[2-(4-chloro-3-fluorophenoxy)acetamido]-2-hydroxybicyclo[2.2.2]octan-1-yl}-2,3-dihydro-1,4-benzodioxine-2-carboxamide (Compound 126)

### Example 27A: N-[(3S)-4-amino-3-hydroxybicyclo[2.2.2]octan-1-yl]-2-(4-chloro-3-fluorophenoxy)acetamide trifluoroacetate

The title compound was isolated by chiral preparative SFC of Example 30E as the second peak eluted off the column, followed by reverse phase HPLC purification to give the title compound as a trifluoroacetic acid salt. The preparative SFC (Supercritical Fluid Chromatography) was performed on a Thar 200 preparative SFC (SFC-5) system using a Chiralpak® IC, 300×5 0 mm I.D., 10 μm column. The column was at 38° C., and the backpressure regulator was set to maintain 100 bar. The mobile phase A is CO₂ and B is isopropanol (0.1% ammonium hydroxide). The chromatography was performed isocratically at 45% of mobile phase B at a flow rate of 200 mL/minute. Fraction collection was time triggered with UV monitor wavelength set at 220 nm. Preparative HPLC was performed on a Gilson 281 semi-preparative HPLC system using a Phenomenex® Luna® C18(2) 10 μm 100 Å AXIA™ column (250 mm×80 mm) column. A gradient of acetonitrile (A) and 0.075% trifluoroacetic acid in water (B) was used, at a flow rate of 80 mL/minute. A linear gradient was used from about 30% of A to about 100% of A over about 30 minutes. Detection method was UV at wave lengths of 220 nM and 254 nM. ¹H NMR (400 MHz, methanol-d₄) δ ppm 7.36 (t, J=8.77 Hz, 1H), 6.89 (dd, J=10.74, 2.85 Hz, 1H), 6.79 (br d, J=9.21 Hz, 1H), 4.43 (s, 2H), 3.94 (br d, J=8.33 Hz, 1H), 2.55 (br t, J=12.50 Hz, 1H), 2.35-1.84 (m, 8H), 1.83-1.58 (m, 2H); MS (ESI⁺) m/z 343.0 (M+H)⁺.

### Example 27B: (2R)—N-{(2S)-4-[2-(4-chloro-3-fluorophenoxy)acetamido]-2-hydroxybicyclo[2.2.2]octan-1-yl}-2,3-dihydro-1,4-benzodioxine-2-carboxamide

To a mixture of Example 27A (45.7 mg, 0.1 mmol), 1-[bis(dimethylamino)methylene]-1H-1,2,3-triazolo[4,5-b]pyridinium 3-oxid hexafluorophosphate (HATU, 41.8 mg, 0.110 mmol) and (R)-2,3-dihydrobenzo[b][1,4]dioxine-2-carboxylic acid (18.02 mg, 0.100 mmol) was added N-ethyl-N-isopropylpropan-2-amine (78 mg, 0.600 mmol) in N,N-dimethylformamide (0.9 mL). The mixture was stirred at room temperature for 1 hour. The mixture was purified by preparative HPLC [Waters XBridge™ C18 5 μm OBD™ column, 30×100 mm, flow rate 40 mL/minute, 5-100% gradient of acetonitrile in buffer (0.1% trifluoroacetic acid in water)] to give the title compound (34 mg, 0.067 mmol, 67% yield). ¹H NMR (400 MHz, DMSO-d₆) δ ppm 7.51 (s, 1H), 7.48 (t, J=8, 1H), 7.24 (s, 1H), 7.04 (dd, J=9, 3 Hz, 1H), 6.95 (m, 1H), 6.80-6.90 (m, 4H), 4.69 (dd, J=6, 3 Hz, 1H), 4.44 (s, 2H), 4.34 (dd, J=11, 3 Hz, 1H), 4.13 (dd, J=11, 6 Hz, 1H), 4.01 (m, 1H), 2.28 (m, 1H), 2.15 (m, 1H), 1.72-1.95 (m, 8H); MS (ESI⁺) m/z 505 (M+H)⁺.

### Example 28: (2S)—N-{(2S)-4-[2-(4-chloro-3-fluorophenoxy)acetamido]-2-hydroxybicyclo[2.2.2]octan-1-yl}-2,3-dihydro-1,4-benzodioxine-2-carboxamide (Compound 127)

The reaction and purification conditions described in Example 27 substituting (S)-2,3-dihydrobenzo[b][1,4]dioxine-2-carboxylic acid for (R)-2,3-dihydrobenzo[b][1,4]dioxine-2-carboxylic acid gave the title compound (32 mg, 60% yield). ¹H NMR (400 MHz, DMSO-d₆) δ ppm 7.51 (s, 1H), 7.48 (t, J=8 Hz, 1H), 7.24 (s, 1H), 7.04 (dd, J=9, 3 Hz, 1H), 6.95 (m, 1H), 6.80-6.90 (m, 4H), 4.69 (dd, J=6, 3 Hz, 1H), 4.44 (s, 2H), 4.34 (dd, J=11, 3 Hz, 1H), 4.13 (dd, J=11, 6 Hz, 1H), 3.91 (m, 1H), 2.30-2.15 (m, 2H), 1.70-1.95 (m, 8H); MS (ESI⁺) m/z 505 (M+H)⁺.

### Example 29: (2R)—N-{3-[2-(4-chloro-3-fluorophenoxy)acetamido]bicyclo[1.1.1]pentan-1-yl}-2,3-dihydro-1H-indole-2-carboxamide (Compound 128)

The product of Example 23B was processed as in Example 23C substituting (2R)-2,3-dihydro-1H-indole-2-carboxylic acid for 2,3-dihydrobenzo[b][1,4]dioxine-2-carboxylic acid to give the title compound. 1H NMR (501 MHz, DMSO-d6-D₂O) δ ppm 7.49 (t, J=8.8 Hz, 1H), 7.14-7.00 (m, 2H), 6.97 (t, J=7.6 Hz, 1H), 6.90-6.83 (m, 1H), 6.68-6.57 (m, 2H), 4.47 (s, 2H), 4.16 (dd, J=10.5, 7.8 Hz, 1H), 3.29 (dd, J=16.3, 10.5 Hz, 1H), 2.98-2.85 (m, 1H), 2.26 (s, 6H); MS (ESI⁺) m/z 430 (M+H)⁺

### Example 30: N-{4-[2-(4-chloro-3-fluorophenoxy)acetamido]-2-hydroxybicyclo[2.2.2]octan-1-yl}-2,3-dihydro-1-benzofuran-2-carboxamide (Compound 129)

### Example 30A: ethyl 4-amino-2-oxobicyclo[2.2.2]octane-1-carboxylate, hydrochloric acid

To a mixture of Example 2D (11.2 g, 33.2 mmol) in tetrahydrofuran (110 mL) in a 250 mL pressure bottle was added 20% Pd(OH)₂/C, wet (2.2 g, 1.598 mmol), and the reaction mixture was shaken at 50° C. under 50 psi of hydrogen for 22 hours. The reaction mixture was cooled to ambient temperature, solids were removed by filtration and washed with methanol (1 L). The filtrate and wash were concentrated to give 7.9 g of the title compound. ¹H NMR (400 MHz, DMSO-d₆) δ ppm 8.46 (s, 3H), 4.07 (q, J=7.1 Hz, 2H), 2.62 (s, 2H), 2.17-2.05 (m, 2H), 2.04-1.78 (m, 6H), 1.14 (t, J=7.1 Hz, 3H).

### Example 30B: ethyl 4-[2-(4-chloro-3-fluorophenoxy)acetamido]-2-oxobicyclo[2.2.2]octane-1-carboxylate

To a suspension of Example 30A (7.8 g, 31.5 mmol), N-ethyl-N-isopropylpropan-2-amine (22.00 mL, 126 mmol) and 2-(4-chloro-3-fluorophenoxy)acetic acid (7.41 g, 36.2 mmol) in N,N-dimethylformamide (200 mL), 2-(3H-[1,2,3]triazolo[4,5-b]pyridin-3-yl)-1,1,3,3-tetramethylisouronium hexafluorophosphate (V) (14.97 g, 39.4 mmol) was added, and the resulting brown solution was stirred at ambient temperature for 16 hours. Water was added, and the mixture was stirred for 15 minutes. The precipitate was collected by filtration, washed with water, and air-dried to give 12.1 g of the title compound. ¹H NMR (400 MHz, DMSO-d₆) δ ppm 7.87 (s, 1H), 7.45 (t, J=8.9 Hz, 1H), 7.00 (dd, J=11.4, 2.9 Hz, 1H), 6.79 (ddd, J=8.9, 2.9, 1.2 Hz, 1H), 4.45 (s, 2H), 4.06 (q, J=7.1 Hz, 2H), 2.73 (s, 2H), 2.07 (m, 1H), 2.01-1.84 (m, 6H), 1.14 (t, J=7.1 Hz, 3H); MS (ESI⁺) m/z 398.0 (M+H)⁺.

### Example 30C: 4-[2-(4-chloro-3-fluorophenoxy)acetamido]-2-oxobicyclo[2.2.2]octane-1-carboxylic acid

A suspension of Example 30B (11.37 g, 28.6 mmol) and sodium hydroxide (7.15 mL, 57.2 mmol, 8 M solution) in methanol (100 mL) was stirred at ambient temperature for 16 hours. Volatiles were removed, and the residue was acidified with 1 N HCl. The precipitate was collected by filtration and dried in vacuum oven to give 9.9 g of the title compound. ¹H NMR (400 MHz, DMSO-d₆) δ ppm 12.49 (s, 1H), 7.86 (s, 1H), 7.45 (t, J=8.9 Hz, 1H), 7.00 (dd, J 11.4, 2.9 Hz, 1H), 6.83-6.74 (m, 1H), 4.45 (s, 2H), 2.71 (s, 2H), 2.01-1.81 (m, 7H); MS (ESI⁻) m/z 368.1 (M−H)⁻.

### Example 30D: N-(4-amino-3-oxobicyclo[2.2.2]octan-1-yl)-2-(4-chloro-3-fluorophenoxy)acetamide

A mixture of Example 30C (3.24 g, 8.76 mmol), diphenylphosphoryl azide (2.84 mL, 13.14 mmol), and triethylamine (3.66 mL, 26.3 mmol) in toluene (100 mL) was heated at reflux for 2 hours. The solution was cooled to ambient temperature and poured into 150 mL of 3 N HCl solution. The mixture was stirred for 16 hours to give a suspension. The precipitate was filtered, washed with ethyl acetate, and air-dried to give the title compound (1.63 g) as an HCl salt. The filtrate was then basified with solid sodium bicarbonate and extracted with ethyl acetate. The organic layer was washed with brine, dried over magnesium sulfate and filtered. The filtrate was concentrated and purified on silica gel (0-10% methanol/dichloromethane) to give the title compound (0.6 g) as the free base. ¹H NMR (400 MHz, DMSO-d₆) δ ppm 8.49 (s, 3H), 8.08 (s, 1H), 7.45 (t, J=8.9 Hz, 1H), 7.01 (dd, J=11.4, 2.8 Hz, 1H), 6.79 (ddd, J=9.0, 2.9, 1.2 Hz, 1H), 4.48 (s, 2H), 2.90 (s, 2H), 2.12-1.79 (m, 8H).

### Example 30E: N-(4-amino-3-hydroxybicyclo[2.2.2]octan-1-yl)-2-(4-chloro-3-fluorophenoxy)acetamide hydrochloride

A mixture of Example 30D (2.5 g, 6.63 mmol) and sodium borohydride (1.254 g, 33.1 mmol) in a 1:1 mixture of methanol/dichloromethane (50 mL) was stirred for 24 hours. Volatiles were removed, and the residue was partitioned between water and dichloromethane. The organic fraction was separated, dried (MgSO₄), and concentrated. The residue was then treated with 4 N HCl in dioxane. The suspension was sonicated and concentrated. The residue was dried under vacuum to give 2.82 g of the title compound. ¹H NMR (400 MHz, DMSO-d₆) δ ppm 7.97 (s, 3H), 7.72 (s, 1H), 7.40 (t, J=8.9 Hz, 1H), 6.95 (dd, J=11.4, 2.8 Hz, 1H), 6.74 (ddd, J=9.0, 2.9, 1.1 Hz, 1H), 5.64 (s, 1H), 4.41 (s, 2H), 3.83 (d, J=9.1 Hz, 1H), 2.24 (td, J=10.8, 9.9, 5.3 Hz, 1H), 1.96-1.51 (m, 9H); MS (ESI⁺) m/z 343.0 (M+H)⁺.

### Example 30F: (2R)—N-{3-[2-(4-chloro-3-fluorophenoxy)acetamido]bicyclo[1.1.1]pentan-1-yl}-2,3-dihydro-1H-indole-2-carboxamide

A 4 mL vial was charged with a stir bar, a 500 μL solution of Example 30E (47.74 mg, 0.13 mmol) in N,N-dimethylacetamide, 2,3-dihydrobenzofuran-2-carboxylic acid (23.11 mg, 0.14 mmol, 1.1 equivalents), 1-[bis(dimethylamino)methylene]-1H-1,2,3-triazolo[4,5-b]pyridinium 3-oxid hexafluorophosphate (HATU, 57.4 mg, 0.15 mmol, 1.2 equivalents), and triethylamine (53.01p L, 0.38 mmol, 3 equivalents). The vial was capped and stirred at room temperature for 1 hour. The mixture was then concentrated to dryness and dissolved in 1.4 mL of dimethyl sulfoxide/methanol (1:1). The crude material was submitted to reverse phase HPLC purification (Phenomenex® Luna® C8(2) 5 μm 100 Å AXIA™ column (30 mm×75 mm). A gradient of acetonitrile (A) and 0.1% trifluoroacetic acid in water (B) was used at a flow rate of 50 mL/minute (0-0.5 minute 10% A, 0.5-6.0 minutes linear gradient 10-100% A, 6.0-7.0 minutes 100% A, 7.0-8.0 minutes linear gradient 100-10% A) to yield the title compound. ¹H NMR (400 MHz, DMSO-d₆) δ ppm 7.47 (t, J=8.9 Hz, 1H), 7.30-7.19 (m, 1H), 7.13 (td, J=7.7, 1.4 Hz, 1H), 7.00 (dd, J=11.4, 2.9 Hz, 1H), 6.94-6.84 (m, 1H), 6.88-6.77 (m, 2H), 5.11-5.01 (m, 1H), 4.42 (s, 2H), 4.07-3.98 (m, 1H), 3.45 (dt, J=16.2, 10.3 Hz, 1H), 3.17 (dt, J=16.2, 8.1 Hz, 1H), 2.35-2.17 (m, 1H), 2.06 (s, 1H), 1.92 (s, 1H), 1.91-1.66 (m, 7H); MS (+ESI) m/z 489.1 (M+H)⁺.

### Example 31: (7S)—N-{3-[2-(4-chloro-3-fluorophenoxy)acetamido]bicyclo[1.1.1]pentan-1-yl}-2,2-difluoro-7-methyl-6,7-dihydro-2H-furo[2,3-f][1,3]benzodioxole-7-carboxamide (Compound 130)

The reaction and purification conditions described in Example 23 substituting (S)-2,2-difluoro-7-methyl-6,7-dihydro-[1,3]dioxolo[4,5-f]benzofuran-7-carboxylic acid for 2,3-dihydrobenzo[b][1,4]dioxine-2-carboxylic acid gave the title compound. ¹H NMR (400 MHz, DMSO-d₆) δ ppm 8.66 (s, 1H), 8.28 (s, 1H), 7.45 (t, J=8.9 Hz, 1H), 7.37 (s, 1H), 7.03 (dd, J=11.4, 2.9 Hz, 1H), 6.95 (s, 1H), 6.81 (ddd, J=9.0, 2.9, 1.2 Hz, 1H), 4.90 (d, J=9.0 Hz, 1H), 4.43 (s, 2H), 4.22 (d, J=9.0 Hz, 1H), 2.19 (s, 6H), 1.46 (s, 3H); MS (ESI⁺) m/z 542 (M+NH₄)⁺.

### Example 32: (7R)—N-{3-[2-(4-chloro-3-fluorophenoxy)acetamido]bicyclo[1.1.1]pentan-1-yl}-2,2-difluoro-7-methyl-6,7-dihydro-2H-furo[2,3-f][1,3]benzodioxole-7-carboxamide (Compound 131)

The reaction and purification conditions described in Example 23 substituting (R)-2,2-difluoro-7-methyl-6,7-dihydro-[1,3]dioxolo[4,5-f]benzofuran-7-carboxylic acid for 2,3-dihydrobenzo[b][1,4]dioxine-2-carboxylic acid gave the title compound. ¹H NMR (400 MHz, DMSO-d₆) δ ppm 8.66 (s, 1H), 8.27 (s, 1H), 7.45 (t, J=8.9 Hz, 1H), 7.37 (s, 1H), 7.03 (dd, J=11.4, 2.9 Hz, 1H), 6.95 (s, 1H), 6.81 (ddd, J=9.0, 2.9, 1.2 Hz, 1H), 4.90 (d, J=9.1 Hz, 1H), 4.43 (s, 2H), 4.22 (d, J=9.0 Hz, 1H), 2.19 (s, 6H), 1.46 (s, 3H); MS (ESI⁺) m/z 542 (M+NH₄)⁺.

### Example 33: N-{3-[(6,7-dichloro-2,3-dihydro-1,4-benzodioxine-2-carbonyl)amino]bicyclo[1.1.1]pentan-1-yl}-5-(trifluoromethoxy)pyridine-2-carboxamide (Compound 132)

The reaction and purification conditions described in Example 1D substituting the product of Example 18D for the product of Example 1C and 5-(trifluoromethoxy)pyridine-2-carboxylic acid (Enamine) for 1,3-benzodioxole-2-carboxylic acid gave the title compound. ¹H NMR (400 MHz, DMSO-d₆) δ ppm 9.35 (s, 1H), 8.85 (s, 1H), 8.71-8.68 (m, 1H), 8.16-8.10 (m, 1H), 8.10-8.05 (m, 1H), 7.24 (s, 1H), 7.22 (s, 1H), 4.83 (dd, J=5.2, 2.9 Hz, 1H), 4.38-4.26 (m, 2H), 2.34 (s, 6H); MS (ESI⁺) m/z 518 (M+H)⁺.

### Example 34: N-{3-[(6,7-dichloro-2,3-dihydro-1,4-benzodioxine-2-carbonyl)amino]bicyclo[1.1.1]pentan-1-yl}-5-(difluoromethyl)pyrazine-2-carboxamide (Compound 133)

The reaction and purification conditions described in Example 18E substituting 5-(difluoromethyl)pyrazine-2-carboxylic acid for 2-(3,4-difluorophenoxy)acetic acid gave the title compound (23 mg, 66% yield). ¹H NMR (400 MHz, DMSO-d₆) δ ppm 9.61 (s, 1H), 9.24 (s, 1H), 8.99 (s, 1H), 8.87 (s, 1H), 7.24 (s, 1H), 7.22 (s, 1H), 7.20 (t, J=56 Hz, 1H), 4.84 (dd, J=5, 3 Hz, 1H), 4.32 (m, 2H), 2.35 (s, 6H); MS (ESI−) m/z 483 (M−H)⁻.

### Example 35: (2R)-6-chloro-N-{3-[2-(4-chloro-3-fluorophenoxy)acetamido]bicyclo[1.1.1]pentan-1-yl}-4-methyl-3,4-dihydro-2H-1,4-benzoxazine-2-carboxamide (Compound 134)

Example 13D was purified by preparative chiral SFC (supercritical fluid chromatography) using a (S,S) Whelk-O®1 column (20×250 mm, 5 micron) eluted with 44% CH₃OH in CO₂ at 33° C. with a CO₂ flow rate of 40 mL/minute, CH₃OH flow rate of 32 mL/minute, front pressure of 192 bar, and back pressure of 100 bar to give the title compound (second enantiomer eluted, 0.0074 g, 0.015 mmol, 39% yield). The absolute stereochemistry of the title compound was arbitrarily assigned. ¹H NMR (400 MHz, DMSO-d₆) δ ppm 8.71 (s, 1H), 8.67 (s, 1H), 7.49 (t, J=8.9 Hz, 1H), 7.07 (dd, J=11.3, 2.9 Hz, 1H), 6.85 (ddd, J=9.0, 2.9, 1.2 Hz, 1H), 6.80 (d, J=8.4 Hz, 1H), 6.70 (d, J=2.4 Hz, 1H), 6.62 (dd, J=8.5, 2.4 Hz, 1H), 4.61 (dd, J=7.3, 3.0 Hz, 1H), 4.47 (s, 2H), 3.40-3.37 (m, 1H), 3.18 (dd, J=12.0, 7.4 Hz, 1H), 2.83 (s, 3H), 2.26 (s, 6H); MS (ESI⁺) m/z 494 (M+H)⁺.

### Example 36: 6-chloro-N-{3-[2-(4-chloro-3-fluorophenoxy)acetamido]bicyclo[1.1.1]pentan-1-yl}-4-(2-hydroxyethyl)-3,4-dihydro-2H-1,4-benzoxazine-2-carboxamide (Compound 135)

To a solution of the product of Example 14 (0.030 g, 0.062 mmol) in N,N-dimethylformamide (0.36 mL) was added K₂CO₃ (0.017 g, 0.13 mmol) and 2-bromoethanol (0.005 mL, 0.07 mmol). The reaction mixture stirred at 70° C. overnight. Then more K₂CO₃ (0.051 g, 0.39 mmol) and 2-bromoethanol (0.015 mL, 0.21 mmol) were added, and the reaction mixture continued to stir at 70° C. for 5 days before cooling to ambient temperature. The mixture was then diluted with N,N-dimethylformamide/water (1.5 mL, 3:1) and purified by preparative HPLC [Waters XBridge™ C18 5 μm OBD column, 30×100 mm, flow rate 40 mL/minute, 5-100% gradient of acetonitrile in buffer (0.025 M aqueous ammonium bicarbonate, adjusted to pH 10 with ammonium hydroxide)] to yield the title compound (0.008 g, 0.015 mmol, 24% yield). ¹H NMR (400 MHz, DMSO-d₆) δ ppm 8.70 (d, J=7.5 Hz, 2H), 7.49 (t, J=8.9 Hz, 1H), 7.07 (dd, J=11.4, 2.8 Hz, 1H), 6.90-6.82 (m, 1H), 6.82-6.71 (m, 2H), 6.54 (dd, J=8.5, 2.4 Hz, 1H), 4.69 (t, J=5.5 Hz, 1H), 4.47 (s, 2H), 4.45 (dd, J=7.9, 2.9 Hz, 2H), 3.61-3.47 (m, 4H), 2.26 (s, 6H), 2.24 (d, J=2.6 Hz, 1H); MS (ESI⁺) m/z 524 (M+H)⁺.

### Example 37: 6-chloro-N-{3-[2-(4-chloro-3-fluorophenoxy)acetamido]bicyclo[1.1.1]pentan-1-yl}-4-(hydroxyacetyl)-3,4-dihydro-2H-1,4-benzoxazine-2-carboxamide (Compound 136)

To a solution of the product of Example 59 (0.027 g, 0.050 mmol) in CH₂Cl₂ (0.5 mL) was added boron tribromide (0.1 mL, 0.1 mmol, 1 M in CH₂Cl₂) in an ice bath. The reaction mixture was allowed to warm to ambient temperature in the ice bath for 40 minutes, was partitioned between water (1 mL) and CH₂Cl₂ (3 mL), and the layers were separated. The aqueous layer was extracted with CH₂Cl₂ (2×10 mL), and the combined organic layers were dried (Na₂SO₄), filtered, and concentrated. The residue was diluted with N,N-dimethylformamide/water (1.2 mL, 3:1) and purified by preparative HPLC [Waters XBridge™ C18 5 μm OBD column, 30×100 mm, flow rate 40 mL/minute, 5-100% gradient of acetonitrile in buffer (0.025 M aqueous ammonium bicarbonate, adjusted to pH 10 with ammonium hydroxide)] to yield the title compound (0.014 g, 0.026 mmol, 52% yield). ¹H NMR (400 MHz, DMSO-d₆) δ ppm 8.83 (s, 1H), 8.70 (s, 1H), 7.92 (s, 1H), 7.49 (t, J=8.9 Hz, 1H), 7.20-6.99 (m, 3H), 6.85 (ddd, J=9.0, 2.9, 1.2 Hz, 1H), 5.10 (t, J=5.8 Hz, 1H), 4.83 (t, J=4.4 Hz, 1H), 4.47 (s, 2H), 4.37-4.21 (m, 2H), 3.96-3.78 (m, 2H), 2.23 (s, 6H); MS (ESI⁺) m/z 538 (M+H)⁺.

### Example 38: 6-chloro-N-{3-[2-(4-chloro-3-fluorophenoxy)acetamido]bicyclo[1.1.1]pentan-1-yl}-4-(1,2-dimethyl-1H-imidazole-5-sulfonyl)-3,4-dihydro-2H-1,4-benzoxazine-2-carboxamide (Compound 137)

To a solution of Example 14 (0.030 g, 0.062 mmol) in dichloromethane (0.21 mL) was added pyridine (10 μL, 0.13 mmol) and 1,2-dimethyl-1H-imidazole-5-sulfonyl chloride (0.018 g, 0.094 mmol). This mixture was allowed to stir at 50° C. overnight and then was concentrated. The residue was diluted with N,N-dimethylformamide (1 mL) and purified by preparative HPLC (Waters XBridge™ C18 5 μm OBD column, 30×100 mm, flow rate 40 mL/minute, 5-100% gradient of acetonitrile in 0.1% trifluoroacetic acid/water) gave the title compound (0.036 g, 0.057 mmol, 91% yield). ¹H NMR (400 MHz, DMSO-d₆) δ ppm 8.84 (s, 1H), 8.69 (s, 1H), 7.59 (s, 1H), 7.51-7.41 (m, 2H), 7.20 (dd, J=8.8, 2.5 Hz, 1H), 7.09-6.99 (m, 2H), 6.82 (ddd, J=8.9, 3.0, 1.2 Hz, 1H), 4.44 (s, 2H), 4.21-4.10 (m, 2H), 3.58 (s, 1H), 3.44 (s, 3H), 2.32 (s, 3H), 2.23 (s, 6H); MS (ESI⁺) m/z 638 (M+H)⁺.

### Example 39: 6-chloro-N-{3-[2-(4-chloro-3-fluorophenoxy)acetamido]bicyclo[1.1.1]pentan-1-yl}-4-[(1R,2S)-2-fluorocyclopropane-1-carbonyl]-3,4-dihydro-2H-1,4-benzoxazine-2-carboxamide (Compound 138)

To a solution of (1R,2S)-2-fluorocyclopropanecarboxylic acid (5.20 mg, 0.050 mmol) in dichloromethane (1 mL) was added 1-chloro-N,N,2-trimethylprop-1-en-1-amine (0.01 mL, 0.1 mmol). After 10 minutes, a solution of Example 14 (0.02 g, 0.042 mmol) in tetrahydrofuran (0.52 mL) and pyridine (0.52 mL) was added. This reaction mixture was allowed to stir at ambient temperature for 2 hours and was concentrated. The residue was diluted with N,N-dimethylformamide/water (1.2 mL, 3:1) and purified by preparative HPLC (Waters XBridge™ C18 5 μm OBD column, 30×100 mm, flow rate 40 mL/minute, 5-100% gradient of acetonitrile in 0.1% trifluoroacetic acid/water) to yield the title compound (0.024 g, 0.042 mmol, quantitative yield). ¹H NMR (400 MHz, DMSO-d₆) δ ppm 8.81 (d, J=7.9 Hz, 1H), 8.71 (d, J=3.6 Hz, 1H), 7.58 (s, 1H), 7.49 (t, J=8.9 Hz, 1H), 7.17 (s, 1H), 7.11-7.01 (m, 2H), 6.85 (dd, J=9.0, 2.9 Hz, 1H), 5.00-4.86 (m, 1H), 4.81 (s, 1H), 4.47 (d, J=1.3 Hz, 2H), 4.35 (dd, J=13.8, 4.5 Hz, 1H), 3.99 (s, 1H), 2.23 (d, J=6.8 Hz, 6H), 2.07 (s, 1H), 1.63-1.45 (m, 1H), 1.36-1.21 (m, 1H). 9 Hz, 1H), 8.71 (d, J=3.6 Hz, 1H), 7.58 (s, 1H), 7.49 (t, J=8.9 Hz, 1H), 7.17 (s, 1H), 7.11-7.01 (m, 2H), 6.85 (dd, J=9.0, 2.9 Hz, 1H), 5.00-4.86 (m, 2H), 4.81 (s, 1H), 4.47 (d, J=1.3 Hz, 2H), 4.35 (dd, J=13.8, 4.5 Hz, 1H), 3.99 (s, 1H), 2.23 (d, J=6.8 Hz, 6H), 1.63-1.45 (m, 1H), 1.36-1.21 (m, 1H); MS (ESI⁺) m/z 566 (M+H)⁺.

### Example 40: 6-chloro-N-{3-[2-(4-chloro-3-fluorophenoxy)acetamido]bicyclo[1.1.1]pentan-1-yl}-4-(2-ethoxyethanesulfonyl)-3,4-dihydro-2H-1,4-benzoxazine-2-carboxamide (Compound 139)

### Example 40A: ethyl 6-chloro-4-((2-ethoxyethyl)sulfonyl)-3,4-dihydro-2H-benzo[b][1,4]oxazine-2-carboxylate

To a mixture of Example 13A (0.080 g, 0.33 mmol) in dichloromethane (1.3 mL) was added triethylamine (0.06 mL, 0.4 mmol) and 2-ethoxyethanesulfonyl chloride (0.063 g, 0.36 mmol). The reaction mixture was stirred at ambient temperature for 1 hour and then was concentrated. The residue was diluted with N,N-dimethylformamide (1 mL) and purified by preparative HPLC [Waters XBridge™ C18 5 μm OBD column, 30×100 mm, flow rate 40 mL/minute, 5-100% gradient of acetonitrile in buffer (0.025 M aqueous ammonium bicarbonate, adjusted to pH 10 with ammonium hydroxide)] to give the title compound (0.019 g, 0.055 mmol, 17% yield). MS (ESI⁺) m/z 378 (M+H)⁺.

### Example 40B: 6-chloro-4-((2-ethoxyethyl)sulfonyl)-3,4-dihydro-2H-benzo[b][1,4]oxazine-2-carboxylic Acid

The methodologies described in Example 81B substituting Example 40A for 81A gave the title compound. ¹H NMR (400 MHz, DMSO-d₆) δ ppm 7.51 (d, J=2.4 Hz, 1H), 7.47 (d, J=2.4 Hz, 0.5H), 7.06 (t, J=2.2 Hz, 1H), 7.04 (t, J=2.3 Hz, 1H), 7.01-6.98 (m, 1H), 6.97 (s, 0.5H), 5.13 (dd, J=5.0, 3.2 Hz, 0.5H), 5.00 (dd, J=5.0, 3.6 Hz, 1H), 4.12 (q, J=7.1 Hz, 1H), 4.02-3.93 (m, 2H), 3.90 (t, J=4.7 Hz, 3H), 3.88-3.79 (m, 4H), 3.39-3.24 (m, 7H), 1.17 (t, J=7.1 Hz, 1H), 0.96 (t, J=7.0 Hz, 3H).

### Example 40C: 6-chloro-N-{3-[2-(4-chloro-3-fluorophenoxy)acetamido]bicyclo[1.1.1]pentan-1-yl}-4-(2-ethoxyethanesulfonyl)-3,4-dihydro-2H-1,4-benzoxazine-2-carboxamide

The methodologies described in Example 14 substituting Example 40B for 6-chloro-3,4-dihydro-2H-benzo[b][1,4]oxazine-2-carboxylic acid gave the title compound. ¹H NMR (501 MHz, DMSO-d₆) δ ppm 8.91 (s, 1H), 8.72 (s, 1H), 7.59 (d, J=2.5 Hz, 1H), 7.49 (t, J=8.9 Hz, 1H), 7.14 (dd, J=8.8, 2.5 Hz, 1H), 7.11-7.02 (m, 2H), 6.90-6.80 (m, 1H), 4.69 (dd, J=8.1, 3.0 Hz, 1H), 4.48 (s, 2H), 4.10 (dd, J=14.0, 2.9 Hz, 1H), 3.69 (td, J=7.5, 5.5 Hz, 3H), 3.67-3.61 (m, 1H), 3.58 (dt, J=14.0, 6.8 Hz, 1H), 3.31-3.27 (m, 2H), 2.27 (s, 6H), 0.98 (t, J=7.0 Hz, 3H); MS (ESI⁺) m/z 616 (M+H)⁺.

### Example 41: 6-chloro-N-{3-[2-(4-chloro-3-fluorophenoxy)acetamido]bicyclo[1.1.1]pentan-1-yl}-4-(1-fluorocyclopropane-1-carbonyl)-3,4-dihydro-2H-1,4-benzoxazine-2-carboxamide (Compound 140)

The methodologies described in Example 39 substituting 1-fluorocyclopropanecarboxylic acid for (1R,2S)-2-fluorocyclopropanecarboxylic acid gave the title compound. ¹H NMR (400 MHz, DMSO-d₆) δ ppm 8.84 (s, 1H), 8.70 (s, 1H), 7.64 (d, J=2.6 Hz, 1H), 7.49 (t, J=8.9 Hz, 1H), 7.16 (dd, J=8.8, 2.5 Hz, 1H), 7.10-7.02 (m, 2H), 6.88-6.81 (m, 1H), 4.92 (dd, J=5.1, 3.3 Hz, 1H), 4.47 (s, 2H), 4.23 (dd, J=13.5, 5.1 Hz, 1H), 4.03 (d, J=11.9 Hz, 1H), 2.33-2.28 (m, 1H), 2.22 (s, 6H), 2.07 (s, 1H), 1.51-1.31 (m, 1H), 1.30-1.13 (m, 1H); MS (ESI⁺) m/z 566 (M+H)⁺.

### Example 42: 6-chloro-N-{3-[2-(4-chloro-3-fluorophenoxy)acetamido]bicyclo[1.1.1]pentan-1-yl}-4-(3,3,4,4,4-pentafluorobutanoyl)-3,4-dihydro-2H-1,4-benzoxazine-2-carboxamide (Compound 141)

The methodologies described in Example 39 substituting 3,3,4,4,4-pentafluorobutanoic acid for (1R,2S)-2-fluorocyclopropanecarboxylic acid gave the title compound. ¹H NMR (400 MHz, DMSO-d₆) δ ppm 8.95 (s, 1H), 8.77 (d, J=1.9 Hz, 1H), 7.56 (t, J=8.9 Hz, 1H), 7.23 (t, J=8.7 Hz, 1H), 7.17-7.09 (m, 2H), 6.91 (ddd, J=8.9, 2.8, 1.2 Hz, 1H), 6.59 (s, 1H), 4.99 (s, 1H), 4.53 (s, 2H), 4.22 (d, J=13.3 Hz, 1H), 4.01 (m, 1H), 3.86 (d, J=14.4 Hz, 2H), 2.28 (d, J=3.6 Hz, 6H); MS (ESI⁺) m/z 640 (M+H)⁺.

### Example 43: rac-(2R)-6-chloro-N-{3-[2-(4-chloro-3-fluorophenoxy)acetamido]bicyclo[1.1.1]pentan-1-yl}-4-[(1R,2R)-2-fluorocyclopropane-1-carbonyl]-3,4-dihydro-2H-1,4-benzoxazine-2-carboxamide (Compound 142)

The methodologies described in Example 39 substituting rac-(1R,2R)-2-fluorocyclopropanecarboxylic acid for (1R,2S)-2-fluorocyclopropanecarboxylic acid gave the title compound (Stereochemistry arbitrarily assigned. Diastereomer of Example 45.). ¹H NMR (400 MHz, DMSO-d₆) δ ppm 8.77 (s, 1H), 8.68 (s, 1H), 7.49 (t, J=8.9 Hz, 1H), 7.17 (d, J=9.0 Hz, 1H), 7.10-7.00 (m, 2H), 6.84 (d, J=8.2 Hz, 1H), 6.51 (s, 1H), 5.07 (s, 1H), 4.89 (d, J=3.8 Hz, 2H), 4.46 (s, 2H), 3.79-3.66 (m, 2H), 2.20 (s, 6H), 1.67 (d, J=22.3 Hz, 1H), 1.15 (t, J=11.1 Hz, 1H); MS (ESI⁺) m/z 566 (M+H)⁺.

### Example 44: 6-chloro-N-{3-[2-(4-chloro-3-fluorophenoxy)acetamido]bicyclo[1.1.1]pentan-1-yl}-4-[4-(trifluoromethoxy)benzoyl]-3,4-dihydro-2H-1,4-benzoxazine-2-carboxamide (Compound 143)

The methodologies described in Example 39 substituting 4-(trifluoromethoxy)benzoic acid for (1R,2S)-2-fluorocyclopropanecarboxylic acid gave the title compound. ¹H NMR (400 MHz, DMSO-d₆) δ ppm 7.65-7.57 (m, 2H), 7.49-7.35 (m, 3H), 7.11-7.01 (m, 3H), 6.97 (dd, J=11.3, 2.8 Hz, 1H), 6.83 (ddd, J=9.0, 2.9, 1.3 Hz, 1H), 4.89 (dd, J=4.3, 3.4 Hz, 1H), 4.43 (s, 2H), 4.26 (dd, J=13.6, 4.3 Hz, 1H), 3.73 (dd, J=13.6, 3.4 Hz, 1H), 2.22 (s, 6H); MS (ESI⁺) m/z 668 (M+H)⁺.

### Example 45: rac-(2R)-6-chloro-N-{3-[2-(4-chloro-3-fluorophenoxy)acetamido]bicyclo[1.1.1]pentan-1-yl}-4-[(1S,2S)-2-fluorocyclopropane-1-carbonyl]-3,4-dihydro-2H-1,4-benzoxazine-2-carboxamide (Compound 144)

The methodologies described in Example 43 provided the diastereomer of Example 43 as the title compound (Stereochemistry arbitrarily assigned.). ¹H NMR (400 MHz, DMSO-d₆) δ ppm 8.89 (s, 1H), 8.71 (s, 1H), 7.82 (s, 1H), 7.49 (t, J=8.9 Hz, 1H), 7.23-7.10 (m, 1H), 7.12-7.00 (m, 2H), 6.85 (ddd, J=9.0, 2.9, 1.2 Hz, 1H), 5.16 (td, J=6.0, 3.2 Hz, 1H), 4.99 (td, J=6.1, 3.3 Hz, 1H), 4.72 (dd, J=7.4, 3.2 Hz, 1H), 4.54-4.26 (m, 2H), 2.32 (d, J=13.1 Hz, 2H), 2.26 (s, 6H), 1.68 (d, J=23.0 Hz, 1H), 1.19 (ddt, J=15.4, 12.7, 5.8 Hz, 1H); MS (ESI⁺) m/z 566 (M+H)⁺.

### Example 46: 6-chloro-N-{3-[2-(4-chloro-3-fluorophenoxy)acetamido]bicyclo[1.1.1]pentan-1-yl}-4-(5-methylfuran-2-carbonyl)-3,4-dihydro-2H-1,4-benzoxazine-2-carboxamide (Compound 145)

The methodologies described in Example 39 substituting 5-methylfuran-2-carboxylic acid for (1R,2S)-2-fluorocyclopropanecarboxylic acid gave the title compound. ¹H NMR (400 MHz, DMSO-d₆) δ ppm 8.39 (d, J=21.0 Hz, 1H), 7.42 (t, J=8.8 Hz, 1H), 7.31 (d, J=2.4 Hz, 1H), 7.13-7.02 (m, 2H), 7.02-6.92 (m, 2H), 6.83 (ddd, J=9.0, 2.9, 1.3 Hz, 1H), 6.28 (dt, J=3.4, 0.9 Hz, 1H), 4.81 (dd, J=5.2, 3.3 Hz, 1H), 4.43 (s, 2H), 4.29 (dd, J=13.7, 5.2 Hz, 1H), 4.00 (dd, J=13.6, 3.3 Hz, 1H), 2.34-2.30 (m, 3H), 2.20 (s, 6H); MS (ESI⁺) m/z 588 (M+H)⁺.

### Example 47: 6-chloro-N-{3-[2-(4-chloro-3-fluorophenoxy)acetamido]bicyclo[1.1.1]pentan-1-yl}-4-(3-methoxybenzoyl)-3,4-dihydro-2H-1,4-benzoxazine-2-carboxamide (Compound 146)

The methodologies described in Example 39 substituting 3-methoxybenzoic acid for (1R,2S)-2-fluorocyclopropanecarboxylic acid gave the title compound. ¹H NMR (400 MHz, DMSO-d₆) δ ppm 7.46-7.32 (m, 2H), 7.14-7.08 (m, 1H), 7.11-7.04 (m, 1H), 7.08-6.99 (m, 4H), 6.98 (dd, J=11.3, 2.9 Hz, 1H), 6.83 (ddd, J=8.9, 2.8, 1.2 Hz, 1H), 4.86 (dd, J=4.5, 3.3 Hz, 1H), 4.43 (s, 2H), 4.25 (dd, J=13.6, 4.5 Hz, 1H), 3.79-3.69 (m, 4H), 2.23 (s, 6H); MS (ESI⁺) m/z 614 (M+H)⁺.

### Example 48: 4-[6-chloro-2-({3-[2-(4-chloro-3-fluorophenoxy)acetamido]bicyclo[1.1.1]pentan-1-yl}carbamoyl)-2,3-dihydro-4H-1,4-benzoxazine-4-carbonyl]benzene-1-sulfonyl fluoride (Compound 147)

The methodologies described in Example 39 substituting 4-(fluorosulfonyl)benzoic acid for (1R,2S)-2-fluorocyclopropanecarboxylic acid gave the title compound. ¹H NMR (400 MHz, DMSO-d₆) δ ppm 8.22-8.14 (m, 2H), 7.90-7.82 (m, 2H), 7.42 (t, J=8.8 Hz, 1H), 7.20 (s, 1H), 7.12 (dd, J=8.8, 2.4 Hz, 1H), 7.06 (d, J=8.8 Hz, 1H), 6.98 (dd, J=11.3, 2.9 Hz, 1H), 6.83 (ddd, J=8.9, 2.8, 1.2 Hz, 1H), 4.91 (t, J=3.8 Hz, 1H), 4.43 (s, 2H), 4.23 (dd, J=13.7, 4.2 Hz, 1H), 3.73 (dd, J=13.7, 3.3 Hz, 1H), 2.23 (s, 6H); MS (ESI⁺) m/z 666 (M+H)⁺.

### Example 49: 6-chloro-N-{3-[2-(4-chloro-3-fluorophenoxy)acetamido]bicyclo[1.1.1]pentan-1-yl}-4-(4,4,4-trifluorobutanoyl)-3,4-dihydro-2H-1,4-benzoxazine-2-carboxamide (Compound 148)

To a solution of Example 14 (0.030 g, 0.062 mmol) in dichloromethane (0.36 mL) was added triethylamine (0.02 mL, 0.13 mmol) and 4,4,4-trifluorobutanoyl chloride (10.53 mg, 0.066 mmol). This mixture was allowed to stir at ambient temperature for 1.5 hours and was concentrated. The residue was diluted with N,N-dimethylformamide/water (1.2 mL, 3:1) and purified by preparative HPLC [Waters XBridge™ C18 5 μm OBD column, 30×100 mm, flow rate 40 mL/minute, 5-100% gradient of acetonitrile in buffer (0.025 M aqueous ammonium bicarbonate, adjusted to pH 10 with ammonium hydroxide)] to give the title compound (0.015 g, 0.025 mmol, 39% yield). ¹H NMR (400 MHz, DMSO-d₆) δ ppm 8.92 (s, 1H), 8.77 (s, 1H), 7.56 (t, J=8.9 Hz, 1H), 7.23 (t, J=8.7 Hz, 1H), 7.18-7.07 (m, 2H), 6.92 (ddd, J=9.0, 2.9, 1.2 Hz, 1H), 6.59 (s, 1H), 4.94 (s, 1H), 4.54 (s, 2H), 4.21 (dd, J=13.9, 4.8 Hz, 1H), 3.88 (dd, J=13.9, 3.3 Hz, 1H), 2.94 (s, 2H), 2.63 (m, 2H), 2.29 (s, 6H); MS (ESI⁺) m/z 604 (M+H)⁺.

### Example 50: 6-chloro-N-{3-[2-(4-chloro-3-fluorophenoxy)acetamido]bicyclo[1.1.1]pentan-1-yl}-4-(cyclopropanecarbonyl)-3,4-dihydro-2H-1,4-benzoxazine-2-carboxamide (Compound 149)

The methodologies described in Example 39 substituting cyclopropanecarboxylic acid for (1R,2S)-2-fluorocyclopropanecarboxylic acid gave the title compound. ¹H NMR (400 MHz, DMSO-d₆) δ ppm 8.41 (d, J=23.7 Hz, 1H), 7.65 (d, J=2.5 Hz, 1H), 7.42 (t, J=8.8 Hz, 1H), 7.10 (dd, J=8.8, 2.5 Hz, 1H), 7.03 (d, J=8.8 Hz, 1H), 6.99 (dd, J=11.3, 2.8 Hz, 1H), 6.84 (ddd, J=9.0, 2.9, 1.3 Hz, 1H), 4.78 (dd, J=5.3, 3.4 Hz, 1H), 4.44 (s, 2H), 4.20 (dd, J=13.7, 5.3 Hz, 1H), 3.96 (dd, J=13.7, 3.5 Hz, 1H), 2.26 (s, 6H), 2.08-1.97 (m, 1H), 0.99-0.82 (m, 4H); MS (ESI⁺) m/z 548 (M+H)⁺.

### Example 51: 6-chloro-N-{3-[2-(4-chloro-3-fluorophenoxy)acetamido]bicyclo[1.1.1]pentan-1-yl}-4-(4-methoxybenzoyl)-3,4-dihydro-2H-1,4-benzoxazine-2-carboxamide (Compound 150)

The methodologies described in Example 39 substituting 4-methoxybenzoic acid for (1R,2S)-2-fluorocyclopropanecarboxylic acid gave the title compound. ¹H NMR (400 MHz, DMSO-d₆) δ ppm 7.46 (s, 1H), 7.47-7.37 (m, 2H), 7.07-6.93 (m, 6H), 6.83 (ddd, J=8.9, 2.8, 1.2 Hz, 1H), 4.85 (dd, J=4.5, 3.3 Hz, 1H), 4.43 (s, 2H), 4.25 (dd, J=13.6, 4.6 Hz, 1H), 3.82 (s, 3H), 3.74 (dd, J=13.6, 3.3 Hz, 1H), 2.22 (s, 6H); MS (ESI⁺) m/z 614 (M+H)⁺.

### Example 52: 6-chloro-N-{3-[2-(4-chloro-3-fluorophenoxy)acetamido]bicyclo[1.1.1]pentan-1-yl}-4-(oxane-4-carbonyl)-3,4-dihydro-2H-1,4-benzoxazine-2-carboxamide (Compound 151)

The methodologies described in Example 39 substituting tetrahydro-2H-pyran-4-carboxylic acid for (1R,2S)-2-fluorocyclopropanecarboxylic acid gave the title compound. ¹H NMR (400 MHz, DMSO-d₆) δ ppm 8.43 (d, J=38.9 Hz, 1H), 7.69 (d, J=2.5 Hz, 1H), 7.42 (t, J=8.8 Hz, 1H), 7.11 (dd, J=8.8, 2.5 Hz, 1H), 7.03 (d, J=8.8 Hz, 1H), 6.98 (dd, J=11.3, 2.8 Hz, 1H), 6.84 (ddd, J=8.9, 2.9, 1.3 Hz, 1H), 4.81 (dd, J=4.8, 3.5 Hz, 1H), 4.44 (s, 2H), 4.20 (dd, J=13.9, 4.8 Hz, 1H), 3.94-3.77 (m, 3H), 3.39 (td, J=11.4, 2.9 Hz, 2H), 3.10 (dq, J=10.3, 5.4, 4.8 Hz, 1H), 2.25 (s, 6H), 1.79-1.60 (m, 4H); MS (ESI⁺) m/z 592 (M+H)⁺.

### Example 53: 6-chloro-N-{3-[2-(4-chloro-3-fluorophenoxy)acetamido]bicyclo[1.1.1]pentan-1-yl}-4-(oxolane-3-carbonyl)-3,4-dihydro-2H-1,4-benzoxazine-2-carboxamide (Compound 152)

The methodologies described in Example 49 substituting tetrahydrofuran-3-carbonyl chloride for 4,4,4-trifluorobutanoyl chloride gave the title compound. ¹H NMR (501 MHz, DMSO-d6, diagnostic peaks) δ ppm 8.82 (s, 1H), 8.70 (s, 1H), 7.49 (t, J=8.8 Hz, 1H), 7.15 (s, 1H), 7.07 (dd, J=11.3, 2.8 Hz, 1H), 7.02 (d, J=8.8 Hz, 2H), 6.85 (d, J=7.0 Hz, 1H), 4.87 (s, 1H), 4.46 (s, 2H), 3.88 (t, J=8.2 Hz, 1H), 3.77 (dd, J=15.1, 7.6 Hz, 3H), 3.58 (s, 2H), 2.22 (s, 6H), 2.06 (d, J=8.8 Hz, 2H); MS (ESI⁺) m/z 578 (M+H)⁺.

### Example 54: 6-chloro-N-{3-[2-(4-chloro-3-fluorophenoxy)acetamido]bicyclo[1.1.1]pentan-1-yl}-4-(5-methyl-1,2-oxazole-4-carbonyl)-3,4-dihydro-2H-1,4-benzoxazine-2-carboxamide (Compound 153)

The methodologies described in Example 39 substituting 5-methylisoxazole-4-carboxylic acid for (1R,2S)-2-fluorocyclopropanecarboxylic acid gave the title compound. ¹H NMR (400 MHz, DMSO-d₆) δ ppm 8.33 (d, J=0.8 Hz, 1H), 7.42 (t, J=8.8 Hz, 1H), 7.30 (d, J=2.4 Hz, 1H), 7.13 (dd, J=8.8, 2.4 Hz, 1H), 7.07 (d, J=8.8 Hz, 1H), 6.97 (dd, J=11.2, 2.8 Hz, 1H), 6.83 (ddd, J=8.9, 2.8, 1.2 Hz, 1H), 4.89 (dd, J=4.2, 3.4 Hz, 1H), 4.43 (s, 2H), 4.29 (dd, J=13.5, 4.2 Hz, 1H), 3.75 (dd, J=13.6, 3.4 Hz, 1H), 2.48 (d, J=0.7 Hz, 3H), 2.20 (s, 6H); MS (ESI⁺) m/z 589 (M+H)⁺.

### Example 55: 6-chloro-N-{3-[2-(4-chloro-3-fluorophenoxy)acetamido]bicyclo[1.1.1]pentan-1-yl}-4-(1,2-oxazole-5-carbonyl)-3,4-dihydro-2H-1,4-benzoxazine-2-carboxamide (Compound 154)

The methodologies described in Example 39 substituting isoxazole-5-carboxylic acid for (1R,2S)-2-fluorocyclopropanecarboxylic acid gave the title compound. ¹H NMR (400 MHz, DMSO-d₆) δ ppm 8.66 (d, J=1.9 Hz, 1H), 7.43 (s, 1H), 7.42 (t, J=8.8 Hz, 1H), 7.18 (dd, J=8.8, 2.5 Hz, 1H), 7.09 (d, J=8.8 Hz, 1H), 6.98 (dd, J=11.3, 2.9 Hz, 1H), 6.90 (d, J=1.9 Hz, 1H), 6.83 (ddd, J=9.0, 2.9, 1.3 Hz, 1H), 4.91 (dd, J=4.6, 3.3 Hz, 1H), 4.43 (s, 2H), 4.31 (dd, J=13.7, 4.7 Hz, 1H), 3.91 (dd, J=13.7, 3.4 Hz, 1H), 2.21 (s, 6H); MS (ESI⁺) m/z 575 (M+H)⁺.

### Example 56: [2-({3-[2-(4-chloro-3-fluorophenoxy)acetamido]bicyclo[1.1.1]pentan-1-yl}carbamoyl)-1,2,3,4-tetrahydroisoquinolin-1-yl]acetic acid (Compound 155)

To a solution of Example 23B (0.040 g, 0.14 mmol), 2-(1,2,3,4-tetrahydroisoquinolin-1-yl)acetic acid (0.028 g, 0.15 mmol), and 4-nitrophenyl carbonochloridate (0.042 g, 0.21 mmol) in acetonitrile (1.4 mL) was added Hunig's Base (N,N-diisopropylethylamine) (0.05 mL, 0.3 mmol) and pyridine (0.06 mL, 0.7 mmol). The reaction mixture was stirred for 35 minutes at 110° C. in a Biotage® Initiator microwave reactor. The reaction mixture was concentrated. The residue was diluted with N,N-dimethylformamide/water (1.2 mL, 3:1), the mixture was filtered and purified by preparative HPLC (Waters XBridge™ C18 5 μm OBD column, 30×100 mm, flow rate 40 mL/minute, 5-100% gradient of acetonitrile in 0.1% trifluoroacetic acid/water) to give the title compound (0.015 g, 0.030 mmol, 21% yield). ¹H NMR (400 MHz, DMSO-d₆) δ ppm 9.49 (br s, 1H), 8.63 (s, 1H), 7.49 (t, J=8.9 Hz, 1H), 7.16-7.00 (m, 5H), 6.85 (ddd, J=8.9, 2.9, 1.2 Hz, 1H), 5.15 (d, J=9.7 Hz, 1H), 4.46 (s, 2H), 3.99 (d, J=12.9 Hz, 1H), 3.58-3.43 (m, 1H), 2.86 (s, 1H), 2.79-2.57 (m, 2H), 2.36-2.29 (m, 1H), 2.15 (s, 6H); MS (ESI⁺) m/z 502 (M+H)⁺.

### Example 57: 6-chloro-N-{3-[2-(4-chloro-3-fluorophenoxy)acetamido]bicyclo[1.1.1]pentan-1-yl}-4-[(2-methoxyethoxy)acetyl]-3,4-dihydro-2H-1,4-benzoxazine-2-carboxamide (Compound 156)

The methodologies described in Example 39 substituting 2-(2-methoxyethoxy)acetic acid for (1R,2S)-2-fluorocyclopropanecarboxylic acid gave the title compound. ¹H NMR (400 MHz, DMSO-d₆) δ ppm 8.44 (d, J=41.4 Hz, 1H), 7.82 (d, J=2.6 Hz, 1H), 7.42 (t, J=8.8 Hz, 1H), 7.11 (dd, J=8.8, 2.5 Hz, 1H), 7.06-6.94 (m, 2H), 6.84 (ddd, J=8.9, 2.9, 1.3 Hz, 1H), 4.79 (dd, J=5.5, 3.4 Hz, 1H), 4.44 (s, 2H), 4.41-4.28 (m, 2H), 3.99 (dd, J=13.9, 5.6 Hz, 1H), 3.87 (dd, J=14.0, 3.4 Hz, 1H), 3.66-3.59 (m, 2H), 3.54-3.47 (m, 2H), 2.26 (s, 6H); MS (ESI⁺) m/z 596 (M+H)⁺.

### Example 58: 4-acetyl-6-chloro-N-{3-[2-(4-chloro-3-fluorophenoxy)acetamido]bicyclo[1.1.1]pentan-1-yl}-3,4-dihydro-2H-1,4-benzoxazine-2-carboxamide (Compound 157)

The methodologies described in Example 49 substituting acetyl chloride for 4,4,4-trifluorobutanoyl chloride and at 0° C. instead of ambient temperature gave the title compound. ¹H NMR (400 MHz, CDCl₃) δ ppm 7.06 (m, 1H), 6.95 (d, J=8.9 Hz, 2H), 6.82 (m, 3H), 6.79-6.63 (m, 2H), 4.72 (m, 1H), 4.39 (s, 4H), 2.48 (s, 6H), 2.33 (s, 3H); MS (ESI⁺) m/z 522 (M+H)⁺.

### Example 59: 6-chloro-N-{3-[2-(4-chloro-3-fluorophenoxy)acetamido]bicyclo[1.1.1]pentan-1-yl}-4-(methoxyacetyl)-3,4-dihydro-2H-1,4-benzoxazine-2-carboxamide (Compound 158)

To a cooled (0° C.) solution of the product of Example 14 (0.030 g, 0.062 mmol) in dichloromethane (0.36 mL) was added triethylamine (0.01 mL, 0.08 mmol) and methoxyacetyl chloride (6 μL, 0.07 mmol). This mixture was allowed to stir at 0° C. for 1 hour. Then the reaction mixture was diluted with water (1 mL) and extracted with dichloromethane (3×1 mL). The combined organic layers were dried over Na₂SO₄, filtered, and concentrated. The residue was redissolved in dichloromethane (2 mL), washed with heptanes, and concentrated to give the title compound (0.035 g, 0.063 mmol, quantitative yield). ¹H NMR (501 MHz, CDCl₃) δ ppm 7.32 (t, J=8.6 Hz, 1H), 7.08 (dd, J=8.8, 2.5 Hz, 1H), 6.96 (d, J=8.8 Hz, 1H), 6.83 (s, 2H), 6.75 (dd, J=10.3, 2.8 Hz, 1H), 6.67 (ddd, J=8.9, 2.9, 1.3 Hz, 1H), 4.73 (t, J=4.6 Hz, 1H), 4.39 (d, J=5.8 Hz, 2H), 4.35 (s, 1H), 4.27-4.17 (m, 1H), 4.09-3.95 (m, 2H), 3.48 (d, J=4.6 Hz, 3H), 2.48 (s, 6H); MS (ESI⁺) m/z 552 (M+H)⁺.

### Example 60: 6-chloro-N-{3-[2-(4-chloro-3-fluorophenoxy)acetamido]bicyclo[1.1.1]pentan-1-yl}-4-(2,2,3,3,4,4,4-heptafluorobutanoyl)-3,4-dihydro-2H-1,4-benzoxazine-2-carboxamide (Compound 159)

The methodologies described in Example 39 substituting 2,2,3,3,4,4,4-heptafluorobutanoic acid for (1R,2S)-2-fluorocyclopropanecarboxylic acid gave the title compound. ¹H NMR (400 MHz, DMSO-d₆) δ ppm 8.50 (d, J=94.0 Hz, 1H), 7.64 (d, J=2.6 Hz, 1H), 7.42 (t, J=8.8 Hz, 1H), 7.25 (dd, J=8.9, 2.5 Hz, 1H), 7.10 (d, J=8.9 Hz, 1H), 6.98 (dd, J=11.2, 2.9 Hz, 1H), 6.84 (ddd, J=9.0, 2.9, 1.3 Hz, 1H), 4.94 (dd, J=4.7, 3.5 Hz, 1H), 4.44 (s, 2H), 4.26 (dd, J=14.2, 4.8 Hz, 1H), 4.04 (dd, J=14.2, 3.5 Hz, 1H), 2.25 (s, 6H); MS (ESI⁺) m/z 676 (M+H)⁺.

### Example 61: 6-chloro-N-{3-[2-(4-chloro-3-fluorophenoxy)acetamido]bicyclo[1.1.1]pentan-1-yl}-4-[3-(trifluoromethyl)benzoyl]-3,4-dihydro-2H-1,4-benzoxazine-2-carboxamide (Compound 160)

The methodologies described in Example 39 substituting 3-(trifluoromethyl)benzoic acid for (1R,2S)-2-fluorocyclopropanecarboxylic acid gave the title compound. ¹H NMR (400 MHz, DMSO-d₆) δ ppm 7.85 (ddd, J=7.5, 2.1, 1.2 Hz, 1H), 7.81-7.74 (m, 2H), 7.74-7.65 (m, 1H), 7.41 (t, J=8.8 Hz, 1H), 7.12-7.01 (m, 2H), 7.04 (s, 1H), 6.97 (dd, J=11.2, 2.8 Hz, 1H), 6.82 (ddd, J=9.0, 2.9, 1.3 Hz, 1H), 4.92 (t, J=3.7 Hz, 1H), 4.42 (s, 2H), 4.31 (dd, J=13.6, 4.1 Hz, 1H), 3.71 (dd, J=13.6, 3.4 Hz, 1H), 2.21 (s, 6H); MS (ESI⁺) m/z 652 (M+H)⁺.

### Example 62: 6-chloro-N-{3-[2-(4-chloro-3-fluorophenoxy)acetamido]bicyclo[1.1.1]pentan-1-yl}-4-(2,2,3,3-tetrafluoropropanoyl)-3,4-dihydro-2H-1,4-benzoxazine-2-carboxamide (Compound 161)

The methodologies described in Example 39 substituting 2,2,3,3-tetrafluoropropanoic acid for (1R,2S)-2-fluorocyclopropanecarboxylic acid gave the title compound. ¹H NMR (400 MHz, DMSO-d₆) δ ppm 7.71 (d, J=2.5 Hz, 1H), 7.42 (t, J=8.8 Hz, 1H), 7.23 (dd, J=8.8, 2.5 Hz, 1H), 7.09 (d, J=8.8 Hz, 1H), 6.98 (dd, J=11.3, 2.8 Hz, 1H), 6.91-6.80 (m, 1H), 6.75 (t, J=5.7 Hz, 1H), 4.91 (dd, J=5.0, 3.5 Hz, 1H), 4.44 (s, 2H), 4.23 (dd, J=14.1, 5.0 Hz, 1H), 4.06 (dd, J=14.1, 3.4 Hz, 1H), 2.25 (s, 6H); MS (ESI⁺) m/z 608 (M+H)⁺.

### Example 63: 6-chloro-N-{3-[2-(4-chloro-3-fluorophenoxy)acetamido]bicyclo[1.1.1]pentan-1-yl}-4-[3-(methylsulfanyl)propanoyl]-3,4-dihydro-2H-1,4-benzoxazine-2-carboxamide (Compound 162)

The methodologies described in Example 39 substituting 3-(methylthio)propanoic acid for (1R,2S)-2-fluorocyclopropanecarboxylic acid gave the title compound. ¹H NMR (400 MHz, DMSO-d₆) δ ppm 7.76 (d, J=2.5 Hz, 1H), 7.42 (t, J=8.8 Hz, 1H), 7.11 (dd, J=8.8, 2.5 Hz, 1H), 7.03 (d, J=8.8 Hz, 1H), 6.98 (dd, J=11.3, 2.8 Hz, 1H), 6.84 (ddd, J=8.9, 2.9, 1.3 Hz, 1H), 4.80 (dd, J=5.2, 3.4 Hz, 1H), 4.44 (s, 2H), 4.09 (dd, J=13.8, 5.2 Hz, 1H), 3.85 (dd, J=13.9, 3.4 Hz, 1H), 2.92-2.79 (m, 2H), 2.82-2.64 (m, 2H), 2.26 (s, 6H), 2.08 (s, 2H); MS (ESI⁺) m/z 582 (M+H)⁺.

### Example 64: 6-chloro-N-{3-[2-(4-chloro-3-fluorophenoxy)acetamido]bicyclo[1.1.1]pentan-1-yl}-4-[5-methyl-2-(trifluoromethyl)furan-3-sulfonyl]-3,4-dihydro-2H-1,4-benzoxazine-2-carboxamide (Compound 163)

To 5-methyl-2-(trifluoromethyl)furan-3-sulfonyl chloride (0.036 g, 0.16 mmol) was added a solution of the product of Example 14 (0.030 g, 0.62 mol) in pyridine (2 mL), and the reaction mixture was heated at 50° C. overnight. The reaction mixture was then cooled to ambient temperature and concentrated. The residue was diluted with dimethyl sulfoxide/methanol (1 mL, 1:1) and purified by preparative HPLC on a Phenomenex® Luna® C8(2) 5 um 100 Å AXIA™ column (30 mm×150 mm). A gradient of acetonitrile (A) and 0.1% trifluoroacetic acid in water (B) was used, at a flow rate of 50 mL/minute (0-0.5 minute 5% A, 0.5-8.5 minutes linear gradient 5-100% A, 8.7-10.7 minutes 100% A, 10.7-11.0 minutes linear gradient 100-5% A) to yield the title compound. ¹H NMR (501 MHz, DMSO-d₆) δ ppm 8.96 (s, 1H), 8.82 (s, 1H), 7.57-7.50 (m, 2H), 7.48 (d, J=8.9 Hz, 1H), 7.24 (dd, J=8.8, 2.5 Hz, 1H), 7.12 (d, J=8.9 Hz, 1H), 7.06 (dd, J=11.3, 2.8 Hz, 1H), 6.86 (ddd, J=9.0, 2.9, 1.2 Hz, 1H), 4.47 (s, 2H), 4.44 (dd, J=8.6, 3.1 Hz, 1H), 4.23 (dd, J=14.2, 3.1 Hz, 1H), 3.73-3.66 (m, 1H), 2.28 (s, 6H); MS (APCI) m/z 692 (M+H)⁺.

### Example 65: 6-chloro-N-{3-[2-(4-chloro-3-fluorophenoxy)acetamido]bicyclo[1.1.1]pentan-1-yl}-4-(4-methoxybenzene-1-sulfonyl)-3,4-dihydro-2H-1,4-benzoxazine-2-carboxamide (Compound 164)

The title compound was prepared following the methodologies described in Example 64, substituting 4-methoxybenzene-1-sulfonyl chloride for 5-methyl-2-(trifluoromethyl)furan-3-sulfonyl chloride. ¹H NMR (501 MHz, DMSO-d₆) δ ppm 8.90 (s, 1H), 8.81 (s, 1H), 7.73-7.65 (m, 2H), 7.61 (d, J=2.5 Hz, 1H), 7.49 (t, J=8.9 Hz, 1H), 7.19 (dd, J=8.8, 2.5 Hz, 1H), 7.16-7.10 (m, 2H), 7.10-7.01 (m, 2H), 6.86 (ddd, J=9.0, 2.9, 1.2 Hz, 1H), 4.47 (s, 2H), 4.25 (d, J=14.3, 3.0 Hz, 1H), 4.00 (d, J=9.1, 3.0 Hz, 1H), 3.83 (s, 3H), 3.56 (dd, J=14.3, 9.1 Hz, 1H), 2.27 (s, 6H); MS (APCI) m/z 650 (M+H)⁺.

### Example 66: 6-chloro-N-{3-[2-(4-chloro-3-fluorophenoxy)acetamido]bicyclo[1.1.1]pentan-1-yl}-4-(1-methyl-1H-imidazole-4-sulfonyl)-3,4-dihydro-2H-1,4-benzoxazine-2-carboxamide (Compound 165)

The title compound was prepared following the methodologies described in Example 64, substituting 1-methyl-1H-imidazole-4-sulfonyl chloride for 5-methyl-2-(trifluoromethyl)furan-3-sulfonyl chloride. ¹H NMR (501 MHz, DMSO-d₆) δ ppm 7.97 (d, J=1.3 Hz, 1H), 7.79 (d, J=1.3 Hz, 1H), 7.67 (d, J=2.5 Hz, 1H), 7.49 (t, J=8.8 Hz, 1H), 7.13 (dd, J=8.8, 2.5 Hz, 1H), 7.09-6.98 (m, 2H), 6.87 (ddd, J=9.0, 2.9, 1.2 Hz, 1H), 4.58-4.52 (m, 1H), 4.49 (d, J=19.8 Hz, 2H), 4.35 (dd, J=14.1, 3.0 Hz, 1H), 3.47 (dd, J=14.1, 9.2 Hz, 1H), 2.29 (s, 6H); MS (APCI) m/z 624 (M+H)⁺.

### Example 67: 6-chloro-N-{3-[2-(4-chloro-3-fluorophenoxy)acetamido]bicyclo[1.1.1]pentan-1-yl}-4-(4-fluorobenzene-1-sulfonyl)-3,4-dihydro-2H-1,4-benzoxazine-2-carboxamide (Compound 166)

The title compound was prepared following the methodologies described in Example 64, substituting 4-fluorobenzene-1-sulfonyl chloride for 5-methyl-2-(trifluoromethyl)furan-3-sulfonyl chloride. ¹H NMR (501 MHz, DMSO-d₆) δ ppm 8.92 (s, 1H), 7.91-7.80 (m, 2H), 7.58 (d, J=2.5 Hz, 1H), 7.48 (td, J=8.8, 4.1 Hz, 3H), 7.20 (dd, J=8.8, 2.5 Hz, 1H), 7.11-7.01 (m, 2H), 6.86 (ddd, J=9.0, 2.9, 1.1 Hz, 1H), 4.47 (s, 2H), 4.27 (dd, J=14.3, 3.1 Hz, 1H), 4.12-4.02 (m, 1H), 3.63 (dd, J=14.3, 8.9 Hz, 1H), 2.27 (s, 6H); MS (APCI) m/z 638 (M+H)⁺.

### Example 68: 6-chloro-N-{3-[2-(4-chloro-3-fluorophenoxy)acetamido]bicyclo[1.1.1]pentan-1-yl}-4-(5,5,5-trifluoropentanoyl)-3,4-dihydro-2H-1,4-benzoxazine-2-carboxamide (Compound 167)

The methodologies described in Example 49 substituting 5,5,5-trifluoropentanoyl chloride for 4,4,4-trifluorobutanoyl chloride gave the title compound. ¹H NMR (400 MHz, DMSO-d₆) δ ppm 8.82 (s, 1H), 8.70 (s, 1H), 7.49 (t, J=8.9 Hz, 1H), 7.23-7.11 (m, 1H), 7.06 (dd, J=11.4, 2.8 Hz, 1H), 7.02 (d, J=8.9 Hz, 2H), 6.84 (ddd, J=9.0, 2.8, 1.2 Hz, 1H), 4.85 (s, 1H), 4.47 (s, 2H), 4.13 (m, 1H), 3.76 (d, J=13.9 Hz, 1H), 2.68 (m, 2H), 2.35-2.24 (m, 2H), 2.22 (s, 6H), 1.76 (p, J=7.1 Hz, 2H); MS (ESI⁺) m/z 618 (M+H)⁺.

### Example 69: 6-chloro-N-{3-[2-(4-chloro-3-fluorophenoxy)acetamido]bicyclo[1.1.1]pentan-1-yl}-4-(methanesulfonyl)-3,4-dihydro-2H-1,4-benzoxazine-2-carboxamide (Compound 168)

The methodologies described in Example 59 substituting methanesulfonyl chloride for methoxyacetyl chloride, at ambient temperature instead of 0° C., and additionally diluting the sample with N,N-dimethylformamide (1 mL) and purifying by preparative HPLC (Waters XBridge™ C18 5 μm OBD column, 30×100 mm, flow rate 40 mL/minute, 5-100% gradient of acetonitrile in 0.1% trifluoroacetic acid/water) gave the title compound. ¹H NMR (400 MHz, DMSO-d₆) δ ppm 8.93 (s, 1H), 8.72 (s, 1H), 7.62 (d, J=2.4 Hz, 1H), 7.49 (t, J=8.9 Hz, 1H), 7.17 (dd, J=8.8, 2.5 Hz, 1H), 7.12-7.01 (m, 2H), 6.85 (d, J=10.7 Hz, 1H), 4.73 (dd, J=7.4, 3.0 Hz, 1H), 4.48 (s, 2H), 4.11-4.06 (m, 1H), 3.67 (dd, J=14.1, 7.7 Hz, 1H), 3.17 (s, 3H), 2.27 (s, 6H); MS (ESI⁺) m/z 558 (M+H)⁺.

### Example 70: 6-chloro-N-{3-[2-(4-chloro-3-fluorophenoxy)acetamido]bicyclo[1.1.1]pentan-1-yl}-4-(pyridine-4-carbonyl)-3,4-dihydro-2H-1,4-benzoxazine-2-carboxamide (Compound 169)

The methodologies described in Example 39 substituting isonicotinic acid for (1R,2S)-2-fluorocyclopropanecarboxylic acid gave the title compound. ¹H NMR (400 MHz, DMSO-d₆) δ ppm 8.73-8.67 (m, 2H), 7.52-7.46 (m, 2H), 7.42 (t, J=8.8 Hz, 1H), 7.25 (s, 1H), 7.15-7.03 (m, 2H), 6.98 (dd, J=11.3, 2.9 Hz, 1H), 6.83 (ddd, J=9.0, 2.9, 1.3 Hz, 1H), 4.90 (dd, J=4.2, 3.4 Hz, 1H), 4.43 (s, 2H), 4.21 (dd, J=13.7, 4.3 Hz, 1H), 3.72 (dd, J=13.7, 3.4 Hz, 1H), 2.23 (s, 6H); MS (ESI⁺) m/z 585 (M+H)⁺.

### Example 71: 3-[6-chloro-2-({3-[2-(4-chloro-3-fluorophenoxy)acetamido]bicyclo[1.1.1]pentan-1-yl}carbamoyl)-2,3-dihydro-4H-1,4-benzoxazin-4-yl]propanoic acid (Compound 170)

### Example 71A: tert-butyl 3-[6-chloro-2-({3-[2-(4-chloro-3-fluorophenoxy)acetamido]bicyclo[1.1.1]pentan-1-yl}carbamoyl)-2,3-dihydro-4H-1,4-benzoxazin-4-yl]propanoate

The methodologies described in Example 36 substituting tert-butyl 3-bromopropanoate for 2-bromoethanol and additionally including acetone (0.4 mL) in the solution gave the title compound. MS (ESI⁺) m/z 554 (M+H)⁺.

### Example 71B: 3-[6-chloro-2-({3-[2-(4-chloro-3-fluorophenoxy)acetamido]bicyclo[1.1.1]pentan-1-yl}carbamoyl)-2,3-dihydro-4H-1,4-benzoxazin-4-yl]propanoic Acid

To a solution of the product of Example 71A (0.0034 g, 0.0056 mmol) in dichloromethane (0.01 mL) was added trifluoroacetic acid (0.0017 mL, 0.022 mmol), and the resulting mixture was stirred at ambient temperature for 4 hours and then concentrated. The residue was diluted with N,N-dimethylformamide (1 mL) and purified by preparative HPLC (Waters XBridge™ C18 5 μm OBD column, 30×100 mm, flow rate 40 mL/minute, 5-100% gradient of acetonitrile in 0.1% trifluoroacetic acid/water) to yield the title compound (0.002 g, 0.0036 mmol, 64% yield). ¹H NMR (400 MHz, DMSO-d₆) δ ppm 8.70 (d, J=9.0 Hz, 2H), 7.49 (t, J=8.9 Hz, 1H), 7.11-7.03 (m, 1H), 6.81 (dt, J=19.4, 10.2 Hz, 3H), 6.59 (d, J=6.1 Hz, 1H), 4.47 (s, 3H), 3.47 (d, J=13.8 Hz, 2H), 2.46 (s, 2H), 2.26 (s, 6H); MS (ESI⁺) m/z 553 (M+H)⁺.

### Example 72: 6-chloro-N-{3-[2-(4-chloro-3-fluorophenoxy)acetamido]bicyclo[1.1.1]pentan-1-yl}-4-[(trifluoromethoxy)acetyl]-3,4-dihydro-2H-1,4-benzoxazine-2-carboxamide (Compound 171)

To Example 14 (0.040 g, 0.074 mmol) was added silver trifluoromethanesulfonate (0.038 g, 0.15 mmol), Selectfluor® (1-chloromethyl-4-fluoro-1,4-diazoniabicyclo[2.2.2]octane bis(tetrafluoroborate)) (0.039 g, 0.11 mmol), and potassium fluoride (0.032 g, 0.22 mmol, 40 weight % loading on alumina). Under nitrogen, to this mixture was added ethyl acetate (0.37 mL), followed by 2-fluoropyridine (0.013 mL, 0.15 mmol) and trimethyl(trifluoromethyl)silane (0.074 mL, 0.15 mmol, 2 M solution in tetrahydrofuran). This mixture was allowed to stir at ambient temperature overnight. The same amount of each reagent was added to the reaction mixture again, and the mixture stirred for another 24 hours. Although the reaction was still incomplete, the reaction mixture was diluted with ethyl acetate, filtered through a silica plug, and concentrated. The residue was diluted with N,N-dimethylformamide/water (0.2 mL) and purified by preparative HPLC [Waters XBridge™ C18 5 μm OBD column, 30×100 mm, flow rate 40 mL/minute, 5-100% gradient of acetonitrile in buffer (0.025 M aqueous ammonium bicarbonate, adjusted to pH 10 with ammonium hydroxide)] to give the title compound (0.005 g, 0.008 mmol, 11% yield). ¹H NMR (500 MHz, DMSO-d₆) δ ppm 8.89 (s, 1H), 8.71 (s, 1H), 8.01-7.75 (m, 1H), 7.49 (t, J=8.9 Hz, 1H), 7.20 (d, J=8.9 Hz, 1H), 7.10-7.02 (m, 2H), 6.87-6.81 (m, 1H), 5.24-5.05 (m, 2H), 4.95-4.89 (m, 1H), 4.47 (s, 2H), 4.07-3.91 (m, 1H), 3.80-3.67 (m, 1H), 2.22 (s, 6H); MS (ESI⁺) m/z 606 (M+H)⁺.

### Example 73: 6-chloro-N-{3-[2-(4-chloro-3-fluorophenoxy)acetamido]bicyclo[1.1.1]pentan-1-yl}-4-(methylsulfanyl)-3,4-dihydro-2H-1,4-benzoxazine-2-carboxamide (Compound 172)

The methodologies described in Example 59 substituting methanesulfinic chloride for methoxyacetyl chloride, without including a wash with heptanes, and additionally diluting the sample with N,N-dimethylformamide (1 mL) and purifying by preparative HPLC (Waters XBridge™ C18 5 μm OBD column, 30×100 mm, flow rate 40 mL/minute, 5-100% gradient of acetonitrile in 0.1% trifluoroacetic acid/water) gave the title compound. ¹H NMR (400 MHz, DMSO-d₆) δ ppm 8.71 (s, 1H), 8.63 (s, 1H), 7.49 (t, J=8.9 Hz, 1H), 7.07 (dd, J=11.4, 2.8 Hz, 1H), 6.89-6.83 (m, 1H), 6.81 (s, 1H), 6.68 (s, 1H), 6.12 (s, 1H), 4.47 (s, 2H), 4.45 (dd, J=7.3, 2.9 Hz, 1H), 3.43 (dt, J=12.1, 3.1 Hz, 1H), 3.17 (dd, J=10.9, 7.2 Hz, 1H), 2.37 (s, 3H), 2.26 (s, 6H); MS (ESI⁺) m/z 526 (M+H)⁺.

### Example 74: 6-chloro-N-{3-[2-(4-chloro-3-fluorophenoxy)acetamido]bicyclo[1.1.1]pentan-1-yl}-4-(1,3-dimethyl-1H-pyrazole-4-sulfonyl)-3,4-dihydro-2H-1,4-benzoxazine-2-carboxamide (Compound 173)

The title compound was prepared following the methodologies described in Example 64, substituting 1,3-dimethyl-1H-pyrazole-4-sulfonyl chloride for 5-methyl-2-(trifluoromethyl)furan-3-sulfonyl chloride. ¹H NMR (501 MHz, DMSO-d₆) δ ppm 8.93 (s, 1H), 8.82 (s, 1H), 8.28 (s, 1H), 7.60 (d, J=2.5 Hz, 1H), 7.49 (t, J=8.9 Hz, 1H), 7.23 (dd, J=8.7, 2.5 Hz, 1H), 7.12-7.03 (m, 2H), 6.89-6.83 (m, 1H), 4.47 (s, 2H), 4.24 (dd, J=14.3, 3.0 Hz, 1H), 4.11 (dd, J=9.6, 3.0 Hz, 1H), 3.46 (dd, J=14.4, 9.5 Hz, 1H), 2.28 (s, 6H), 2.08 (s, 3H); MS (ESI⁺) m/z 638 (M+H)⁺.

### Example 75: 6-chloro-N-{3-[2-(4-chloro-3-fluorophenoxy)acetamido]bicyclo[1.1.1]pentan-1-yl}-4-(4-sulfamoylbenzene-1-sulfonyl)-3,4-dihydro-2H-1,4-benzoxazine-2-carboxamide (Compound 174)

The title compound was prepared following the methodologies described in Example 64, substituting 4-sulfamoylbenzene-1-sulfonyl chloride for 5-methyl-2-(trifluoromethyl)furan-3-sulfonyl chloride. ¹H NMR (501 MHz, DMSO-d₆) δ ppm 8.94 (s, 1H), 8.82 (s, 1H), 8.09-7.97 (m, 4H), 7.58-7.44 (m, 2H), 7.20 (dd, J=8.8, 2.5 Hz, 1H), 7.11-7.01 (m, 2H), 6.91-6.82 (m, 1H), 4.47 (s, 2H), 4.30 (dd, J=14.2, 3.1 Hz, 1H), 4.18 (dd, J=8.6, 3.0 Hz, 1H), 2.27 (s, 6H); MS (APCI) m/z 698 (M+H)⁺.

### Example 76: 6-chloro-N-{3-[2-(4-chloro-3-fluorophenoxy)acetamido]bicyclo[1.1.1]pentan-1-yl}-4-[(1S,2S)-2-fluorocyclopropane-1-carbonyl]-3,4-dihydro-2H-1,4-benzoxazine-2-carboxamide (Compound 175)

The methodologies described in Example 39 also prepared this diastereomer of Example 39 as the title compound. ¹H NMR (501 MHz, DMSO-d₆) δ ppm 9.97 (s, 1H), 8.96 (s, 1H), 8.73 (s, 1H), 8.11 (d, J=2.5 Hz, 1H), 7.50 (t, J=8.9 Hz, 1H), 7.19 (dd, J=8.7, 2.6 Hz, 1H), 7.13-7.04 (m, 2H), 6.86 (ddd, J=9.0, 2.8, 1.1 Hz, 1H), 5.42 (d, J=2.5 Hz, 1H), 4.96-4.77 (m, 1H), 4.50 (d, J=2.5 Hz, 1H), 4.48 (s, 2H), 2.69-2.54 (m, 1H), 2.31 (s, 6H), 1.59-1.47 (m, 1H), 1.23 (dq, J=13.0, 6.4 Hz, 1H); MS (ESI⁺) m/z 566 (M+H)⁺.

### Example 77: 6-chloro-N-{3-[2-(4-chloro-3-fluorophenoxy)acetamido]bicyclo[1.1.1]pentan-1-yl}-4-(2,2-difluorocyclopropane-1-carbonyl)-3,4-dihydro-2H-1,4-benzoxazine-2-carboxamide (Compound 176)

The methodologies described in Example 39 substituting 2,2-difluorocyclopropane-carboxylic acid for (1R,2S)-2-fluorocyclopropanecarboxylic acid gave the title compound. ¹H NMR (400 MHz, DMSO-d₆) δ ppm 8.79 (s, 1H), 8.69 (s, 1H), 7.49 (t, J=8.8 Hz, 1H), 7.20 (s, 1H), 7.10-7.01 (m, 2H), 6.84 (d, J=9.8 Hz, 1H), 4.92 (s, 1H), 4.46 (s, 2H), 3.70 (m, 2H), 2.20 (s, 6H), 1.99 (m, 2H), 1.24 (m, 1H); MS (ESI⁺) m/z 585 (M+H)⁺.

### Example 78: 6-chloro-N-{3-[2-(4-chloro-3-fluorophenoxy)acetamido]bicyclo[1.1.1]pentan-1-yl}-4-[1-(trifluoromethyl)cyclopropane-1-carbonyl]-3,4-dihydro-2H-1,4-benzoxazine-2-carboxamide (Compound 177)

The methodologies described in Example 39 substituting 1-(trifluoromethyl)-cyclopropanecarboxylic acid for (1R,2S)-2-fluorocyclopropanecarboxylic acid gave the title compound. ¹H NMR (501 MHz, CDCl₃) δ ppm 8.02 (s, 1H), 7.33 (t, J=8.6 Hz, 1H), 7.18 (dd, J=8.8, 2.4 Hz, 1H), 7.01 (d, J=8.8 Hz, 1H), 6.91 (s, 1H), 6.86 (s, 1H), 6.76 (dd, J=10.2, 2.9 Hz, 1H), 6.68 (ddd, J=8.9, 2.9, 1.3 Hz, 1H), 4.70 (dd, J=8.4, 3.0 Hz, 1H), 4.49 (d, J=13.9 Hz, 1H), 4.40 (s, 2H), 3.78 (s, 1H), 2.52 (s, 6H), 1.44-1.21 (m, 2H), 0.94-0.72 (m, 2H); MS (ESI⁺) m/z 616 (M+H)⁺.

### Example 79: 6-chloro-N-{3-[2-(4-chloro-3-fluorophenoxy)acetamido]bicyclo[1.1.1]pentan-1-yl}-4-(2,2,3,3,3-pentafluoropropanoyl)-3,4-dihydro-2H-1,4-benzoxazine-2-carboxamide (Compound 178)

The methodologies described in Example 39 substituting 2,2,3,3,3-pentafluoropropanoic acid for (1R,2S)-2-fluorocyclopropanecarboxylic acid gave the title compound. ¹H NMR (500 MHz, DMSO-d₆) δ ppm 8.96 (s, 1H), 8.72 (s, 1H), 7.78 (s, 1H), 7.49 (t, J=8.9 Hz, 1H), 7.29 (d, J=8.3 Hz, 1H), 7.12-7.04 (m, 2H), 6.85 (dd, J=8.7, 2.7 Hz, 1H), 5.02 (d, J=3.9 Hz, 1H), 4.47 (s, 2H), 4.14 (m, 2H), 2.22 (s, 6H), 1.24 (m, 2H), 1.16-0.97 (m, 2H); MS (ESI⁺) m/z 626 (M+H)⁺.

### Example 80: 6-chloro-N-{3-[2-(4-chloro-3-fluorophenoxy)acetamido]bicyclo[1.1.1]pentan-1-yl}-4-(2,2-difluoro-1-methylcyclopropane-1-carbonyl)-3,4-dihydro-2H-1,4-benzoxazine-2-carboxamide (Compound 179)

The methodologies described in Example 39 substituting 2,2-difluoro-1-methylcyclopropanecarboxylic acid for (1R,2S)-2-fluorocyclopropanecarboxylic acid gave the title compound. ¹H NMR (501 MHz, DMSO-d₆) δ ppm 8.84 (s, 1H), 8.70 (s, 1H), 7.49 (t, J=8.9 Hz, 1H), 7.23-7.17 (m, 1H), 7.10-7.03 (m, 2H), 6.84 (ddd, J=9.0, 2.8, 1.2 Hz, 1H), 6.51 (s, 1H), 4.97 (t, J=3.8 Hz, 1H), 4.46 (s, 2H), 2.22 (s, 6H), 2.07 (s, 3H), 1.71 (m, 1H), 1.32 (m, 1H); MS (ESI⁺) m/z 598 (M+H)⁺.

### Example 81: 6-chloro-N-{3-[2-(4-chloro-3-fluorophenoxy)acetamido]bicyclo[1.1.1]pentan-1-yl}-4-[2-(methanesulfonyl)ethyl]-3,4-dihydro-2H-1,4-benzoxazine-2-carboxamide (Compound 180)

### Example 81A: ethyl 6-chloro-4-(2-(methylsulfonyl)ethyl)-3,4-dihydro-2H-benzo[b][1,4]oxazine-2-carboxylate

To a mixture of Example 13A (0.080 g, 0.33 mmol) and K₂CO₃ (0.11 g, 0.83 mmol) in acetone (1.3 mL) was added (methylsulfonyl)ethene (0.072 mL, 0.828 mmol). The reaction mixture was heated to 65° C. for 21 hours, cooled to ambient temperature, and concentrated. The residue was diluted with N,N-dimethylformamide/water (1.2 mL, 3:1) and purified by preparative HPLC [Waters XBridge™ C18 5 μm OBD column, 30×100 mm, flow rate 40 mL/minute, 5-100% gradient of acetonitrile in buffer (0.025 M aqueous ammonium bicarbonate, adjusted to pH 10 with ammonium hydroxide)] to yield the title compound (0.019 g, 0.055 mmol, 7% yield). ¹H NMR (501 MHz, DMSO-d₆) δ ppm 6.84-6.75 (m, 2H), 6.65 (dd, J=8.5, 2.5 Hz, 1H), 5.02 (t, J=3.8 Hz, 1H), 4.14 (q, J=7.1 Hz, 2H), 3.78-3.72 (m, 1H), 3.68-3.58 (m, 3H), 3.51 (dd, J=7.7, 3.9 Hz, 2H), 3.04 (s, 3H), 1.19 (t, J=7.1 Hz, 3H); MS (ESI⁺) m/z 348 (M+H)⁺.

### Example 81B: 6-chloro-4-(2-(methylsulfonyl)ethyl)-3,4-dihydro-2H-benzo[b][1,4]oxazine-2-carboxylic Acid

To a solution of Example 81A (0.019 g, 0.055 mmol) in methanol/water (0.054 mL, 1:1) was added NaOH (5 N aqueous solution, 0.055 mL), and the mixture stirred for 1 hour. The mixture was concentrated and the residue was acidified with HCl (1 N). The resultant mixture was concentrated again and carried forward without purification.

### Example 81C: 6-chloro-N-{3-[2-(4-chloro-3-fluorophenoxy)acetamido]bicyclo[1.1.1]pentan-1-yl}-4-[2-(methanesulfonyl)ethyl]-3,4-dihydro-2H-1,4-benzoxazine-2-carboxamide

The methodologies described in Example 14 substituting Example 81B for 6-chloro-3,4-dihydro-2H-benzo[b][1,4]oxazine-2-carboxylic acid gave the title compound (0.005 g, 0.009 mmol, 16% yield). ¹H NMR 1H NMR (500 MHz, DMSO-d₆) δ ppm 8.71 (s, 2H), 7.49 (td, J=8.8, 2.1 Hz, 1H), 7.05-7.00 (m, 1H), 6.88-6.79 (m, 3H), 6.65 (dd, J=8.5, 2.3 Hz, 1H), 4.52 (dd, J=7.6, 2.9 Hz, 1H), 4.48 (s, 2H), 4.46 (s, 1H), 3.81-3.72 (m, 1H), 3.67 (d, J=7.0 Hz, 1H), 3.50 (dd, J=12.3, 2.9 Hz, 1H), 3.30-3.26 (m, 2H), 3.03 (s, 3H), 2.26 (s, 6H); MS (ESI⁺) m/z 586 (M+H)⁺.

### Example 82: 6-chloro-N-{3-[2-(4-chloro-3-fluorophenoxy)acetamido]bicyclo[1.1.1]pentan-1-yl}-4-(3-methoxypropanoyl)-3,4-dihydro-2H-1,4-benzoxazine-2-carboxamide (Compound 181)

The methodologies described in Example 49 substituting 3-methoxypropanoyl chloride for 4,4,4-trifluorobutanoyl chloride gave the title compound. ¹H NMR (501 MHz, DMSO-d₆) δ ppm 8.82 (s, 1H), 8.71 (s, 1H), 7.49 (t, J=8.8 Hz, 1H), 7.15 (d, J=9.0 Hz, 1H), 7.06 (dd, J=11.3, 2.9 Hz, 2H), 7.02 (d, J=8.8 Hz, 1H), 6.89-6.80 (m, 1H), 4.81 (s, 1H), 4.47 (s, 2H), 4.05 (dd, J=13.8, 5.3 Hz, 1H), 3.86 (dd, J=13.9, 3.3 Hz, 1H), 3.60 (t, J=7.0 Hz, 2H), 3.25 (s, 3H), 2.80 (m, 1H), 2.24 (m, 1H), 2.23 (s, 6H); MS (ESI⁺) m/z 566 (M+H)⁺.

### Example 83: 6-chloro-N-{3-[2-(4-chloro-3-fluorophenoxy)acetamido]bicyclo[1.1.1]pentan-1-yl}-4-(3,3,3-trifluoropropanoyl)-3,4-dihydro-2H-1,4-benzoxazine-2-carboxamide (Compound 182)

The methodologies described in Example 49 substituting 3,3,3-trifluoropropanoyl chloride for 4,4,4-trifluorobutanoyl chloride gave the title compound. ¹H NMR (501 MHz, DMSO-d₆) δ ppm 8.90 (d, J=25.4 Hz, 1H), 8.70 (s, 1H), 7.49 (t, J=8.9 Hz, 1H), 7.21 (d, J=24.7 Hz, 1H), 7.14-6.97 (m, 3H), 6.84 (dd, J=9.2, 3.0 Hz, 1H), 4.94 (m, 1H), 4.46 (s, 2H), 4.13 (m, 1H), 3.80 (m, 1H), 3.41 (m, 2H), 2.22 (s, 6H); MS (ESI⁺) m/z 590 (M+H)⁺.

### Example 84: 6-chloro-N-{3-[2-(4-chloro-3-fluorophenoxy)acetamido]bicyclo[1.1.1]pentan-1-yl}-4-(2,2,2-trifluoroethanesulfonyl)-3,4-dihydro-2H-1,4-benzoxazine-2-carboxamide (Compound 183)

The methodologies described in Example 69 substituting 2,2,2-trifluoroethanesulfonyl chloride for methanesulfonyl chloride gave the title compound. ¹H NMR (400 MHz, DMSO-d₆) δ ppm 8.90 (s, 1H), 8.69 (s, 1H), 7.56 (d, J=2.4 Hz, 1H), 7.46 (t, J=8.9 Hz, 1H), 7.17 (dd, J=8.9, 2.4 Hz, 1H), 7.08-7.00 (m, 2H), 6.82 (ddd, J=8.9, 2.8, 1.2 Hz, 1H), 5.05-4.78 (m, 2H), 4.71 (dd, J=7.6, 3.1 Hz, 1H), 4.44 (s, 2H), 4.10 (dd, J=14.2, 3.1 Hz, 1H), 3.65 (dd, J=14.2, 7.7 Hz, 1H), 2.24 (s, 6H); MS (ESI⁺) m/z 626 (M+H)⁺.

### Example 85: tert-butyl [(2S)-6-chloro-2-({(3S)-4-[2-(4-chloro-3-fluorophenoxy)acetamido]-3-hydroxybicyclo[2.2.2]octan-1-yl}carbamoyl)-2,3-dihydro-4H-1,4-benzoxazin-4-yl]acetate (Compound 184)

### Example 85A: I-amino-4-(benzylamino)bicyclo[2.2.2]octan-2-one, hydrochloric Acid

To a suspension of Example 2E (10.01 g, 32.3 mmol) in toluene (100 mL) was added a 50% ethyl acetate solution of 2,4,6-tripropyl-1,3,5,2,4,6-trioxatriphosphinane 2,4,6-trioxide (22 mL, 37.0 mmol), trimethylsilyl azide (TMS-N₃) (5.0 mL, 37.7 mmol), and triethylamine (11.5 mL, 83 mmol). The mixture was stirred for 30 minutes at room temperature, heated for 2 hours at 85° C., and then 3 N aqueous hydrochloric acid (86 mL, 258 mmol) was added. The mixture was stirred at 85° C. for 90 minutes and then concentrated. The concentrate was stirred with acetonitrile (150 mL) to precipitate a white solid, which was collected by filtration, washed with acetonitrile (30 mL) and CH₂Cl₂ (25 mL), and vacuum-dried to provide the title compound as an HCl salt (6.244 g, 60.9% yield). MS (APCI⁺) m/z 245.0 (M+H)⁺.

### Example 85B: tert-butyl (S)-(4-(benzylamino)-2-hydroxybicyclo[2.2.2]octan-1-yl)carbamate hydrochloride

Water (3.24 L) was charged to a 6 L jacketed reaction vessel, followed by magnesium chloride heptahydrate (1.46 g, 7.1 mmol), NADP⁺ (1.8 g, 2.3 mmol) and monopotassium phosphate (6.93 g, 526 mmol). All components were dissolved before the pH was adjusted to pH 7.5 with 50% weight/weight NaOH to make desired buffer (200 mL reserved). The product of Example 85A (180 g, 569 mmol) was added to the buffer, and the pH was again adjusted to pH 7.5 with 50% weight/weight NaOH. Finally, isopropanol (360 mL, 10% volume/volume) was added to the reaction followed by enzyme (3.6 g, KRED P2C02, Codexis, Redwood City, CA) that was solubilized in the reserved 200 mL of buffer. This reaction was allowed to proceed at 40° C. for 16 hours with the pH being held between 7.5 and 8.0. Upon completion of the reaction, the buffer was adjusted to pH 12 and held at this point for 30 minutes. The reaction was filtered through diatomaceous earth to remove the enzyme. Di-tert-butyl dicarbonate (20.7 g, 98 mmol, 1.25 equivalents) was added to 3.6 L of ethyl acetate, and the solution was charged to the reaction vessel containing the filtered aqueous fraction. This reaction was allowed to proceed at 30° C. for 2.5 hours with moderate stirring. After 2.5 hours, the two layers were separated, and the aqueous fraction was assayed for remaining amino-alcohol product. Di-tert-butyl dicarbonate (1.25 eq. with respect to the remaining amino-alcohol) was added to 2.0 L of ethyl acetate and charged to the reaction vessel. The reaction was allowed to proceed at 30° C. for 2.5 hours, after which the organic and aqueous layers were separated. The combined organic layers were washed with 2.5% NaOH (560 mL) and dried over Na₂SO₄. The ethyl acetate was removed in vacuo and the residue taken up in methyl tert-butyl ether (MTBE) (1.8 L) to provide the product in solution at 0.2 M. This reaction was fitted with an overhead stirrer, cooled to 0° C. and 4 N HCl in dioxane (169 mL, 1.5 eq with respect to product) was slowly added. The product precipitated out of solution and, after stirring for 5 minutes at 0° C., was collected by filtration. The white solid was washed with methyl tert-butyl ether (MTBE) (100 mL) and dried under vacuum overnight to provide the title compound (160 g, 418 mmol, 79% yield). MS (APCI⁺) m/z 347.4 (M+H)⁺.

### Example 85C: tert-butyl (S)-(4-amino-2-hydroxybicyclo[2.2.2]octan-1-yl)carbamate hydrochloride

The product of Example 85B (163.7 g, 427 mmol) in methanol (1375 mL) was added to 20% Pd(OH)₂/C, wet (16 g, 58 mmol) in a 2 L Hastelloy® C reactor. The reactor was purged with argon and was stirred under 50 psi of hydrogen at 40° C. The reaction mixture was allowed to stir for 11.3 hours after reaching 38° C. The reactor was vented and materials were filtered through 45 m nylon filter to remove catalyst and concentrated under reduced pressure to give the title compound (121.49 g, 415 mmol, 97% yield). ¹H NMR (400 MHz, DMSO-d₆) δ ppm 8.14 (s, 3H), 6.12 (s, 1H), 5.09 (d, J=4.2 Hz, 1H), 3.90 (dt, J=9.4, 3.0 Hz, 1H), 2.11 (ddd, J=12.8, 9.5, 3.0 Hz, 1H), 2.06-1.94 (m, 1H), 1.88-1.50 (m, 7H), 1.32 (s, 9H).

### Example 85D: (R)-4-chloro-2-((3-chloro-2-hydroxypropyl)amino)phenol

To a solution of 2-amino-4-chlorophenol (25 g, 174 mmol) in ethanol (250 mL) and water (2.5 mL) was added (R)-2-(chloromethyl)oxirane (17.7 g, 192 mmol), and the solution stirred for 12 hours at 60° C. Then the reaction mixture was concentrated, and the crude residue was chromatographed on silica gel (ethyl acetate/petroleum ether 1:5) to give the title compound (64 g, 217 mmol, 62% yield). ¹H NMR (400 MHz, CDCl₃) δ ppm 3.17-3.39 (m, 2H) 3.60-3.75 (m, 2H) 4.14 (d, J=7.06 Hz, 1H) 6.55-6.66 (m, 3H).

### Example 85E: (S)-(6-chloro-3,4-dihydro-2H-benzo[b][1,4]oxazin-2-yl)methanol

To a solution of Example 85D (51 g, 173 mmol) in ethanol (500 mL) was added K₂CO₃ (14.3 g, 104 mmol), and the solution was stirred for 12 hours at 90° C. The reaction solution was filtered and concentrated under reduced pressure. The mixture was diluted with water (1000 mL) and extracted with ethyl acetate (3×1000 mL). The combined organic layers were washed with brine, dried over Na₂SO₄, filtered, and concentrated under reduced pressure. The residue was chromatographed on silica gel (ethyl acetate/petroleum ether, 1:4) to give the title compound (35 g, 158 mmol, 46% yield). ¹H NMR (400 MHz, CDCl₃) δ ppm 3.26-3.41 (m, 2H) 3.77-3.89 (m, 3H) 4.17-4.24 (m, 1H) 6.57-6.64 (m, 1H) 6.57-6.64 (m, 1H) 6.73 (d, J=8.38 Hz, 1H).

### Example 85F: (S)-tert-butyl 2-(((tert-butoxycarbonyl)oxy)methyl)-6-chloro-2H-benzo[b][1,4]oxazine-4(3H)-carboxylate

To a solution of Example 85E (15 g, 68 mmol) in dichloromethane (150 mL) was added triethylamine (23.6 mL, 169 mmol), di-tert-butyl dicarbonate (63 mL, 270 mmol) and 4-(dimethylamino)pyridine (0.83 g, 6.8 mmol). The solution stirred for 2 hours at 20° C. Then water (500 mL) was added to the mixture, and the aqueous mixture was extracted with dichloromethane (2×500 mL). The combined organic layers were washed with brine (300 mL), dried over Na₂SO₄, filtered, and concentrated. The residue was purified by silica gel chromatography eluted with petroleum ether:ethyl acetate (10:1) to give the title compound (45 g, 101 mmol, 75% yield). ¹H NMR (400 MHz, CDCl₃) δ ppm 1.50 (s, 9H) 1.54-1.56 (m, 9H) 3.48 (br dd, J=13.56, 7.83 Hz, 1H) 4.14-4.19 (m, 1H) 4.25 (d, J=5.29 Hz, 2H) 4.37 (dtd, J=7.75, 5.17, 5.17, 2.65 Hz, 1H) 6.84 (d, J=8.82 Hz, 1H) 6.95 (dd, J=8.71, 2.54 Hz, 1H) 7.84 (br s, 1H).

### Example 85G: (S)-tert-butyl 6-chloro-2-(hydroxymethyl)-2H-benzo[b][1,4]oxazine-4(3H)-carboxylate

To a solution of Example 85F (45 g, 101 mmol) in methanol (400 mL) and tetrahydrofuran (400 mL) was added a solution of NaOH (8.10 g, 203 mmol) in water (400 mL) at 20° C., and the mixture was stirred at 20° C. for 12 hours. The mixture was concentrated and extracted with ethyl acetate (3×500 mL). The combined organic layers were washed with brine (300 mL), dried over Na₂SO₄, and concentrated to give the title compound (30 g, 93 mmol, 86% yield). ¹H NMR (400 MHz, CDCl₃) δ ppm 1.55 (s, 9H) 3.56 (dd, J=13.81, 7.67 Hz, 1H) 3.80 (br s, 2H) 4.07 (dd, J=14.03, 2.63 Hz, 1H) 4.25 (dtd, J=7.67, 4.93, 4.93, 2.63 Hz, 1H) 6.82 (d, J=8.77 Hz, 1H) 6.95 (dd, J=8.77, 2.19 Hz, 1H) 7.79 (br s, 1H).

### Example 85H: (S)-4-(tert-butoxycarbonyl)-6-chloro-3,4-dihydro-2H-benzo[b][1,4]oxazine-2-carboxylic ACID

To a solution of Example 85G (25 g, 78 mmol) in dichloromethane (250 mL) was added N-methylmorpholine-N-oxide (NMO, 54.5 g, 465 mmol) at 0° C. and tetrapropylammonium perruthenate (TPAP, 5.45 g, 15.51 mmol) at 0° C. The solution was stirred for 2 hours at 20° C. and then was concentrated. The residue was purified by preparative HPLC (Waters XBridge™ C18 5 μm OBD column, 30×100 mm, flow rate 40 mL/minute, 5-100% gradient of methanol in 0.1% trifluoroacetic acid/water) to give the title compound (24 g, 76 mmol, 81% yield). ¹H NMR (400 MHz, CDCl₃) δ ppm 1.53 (s, 9H) 3.91 (dd, J=13.94, 3.18 Hz, 1H) 4.24 (dd, J=13.94, 5.14 Hz, 1H) 4.88 (dd, J=4.89, 3.42 Hz, 1H) 6.91-6.95 (m, 1H) 6.97-7.02 (m, 1H) 7.80 (br s, 1H).

### Example 85I: (S)-6-chloro-3,4-dihydro-2H-benzo[b][1,4]oxazine-2-carboxylic Acid

To a solution of Example 85H (12 g, 38 mmol) in dichloromethane (120 mL) was added HCl (37.8 mL, 151 mmol, ethyl acetate) at 0° C. The solution stirred for 12 hours at 20° C., and then the solid was collected by suction filtration and was dried to give the title compound (15.9 g, 72.4 mmol, 96% yield). ¹H NMR (400 MHz, DMSO-d₆) δ ppm 3.34-3.44 (m, 2H) 4.82 (t, J=3.67 Hz, 1H) 6.50 (dd, J=8.80, 2.45 Hz, 1H) 6.59 (d, J=2.45 Hz, 1H) 6.71 (d, J=8.80 Hz, 1H).

### Example 85J. tert-butyl ((S)-4-((S)-6-chloro-3,4-dihydro-2H-benzo[b][1,4]oxazine-2-carboxamido)-2-hydroxybicyclo[2.2.2]octan-1-yl)carbamate

The methodologies described in Example 14 substituting Example 851 for 6-chloro-3,4-dihydro-2H-benzo[b][1,4]oxazine-2-carboxylic acid and substituting 85C for Example 23B gave the title compound. MS (ESI⁺) m/z 452 (M+H)⁺.

### Example 85K: (S)—N—((S)-4-amino-3-hydroxybicyclo[2.2.2]octan-1-yl)-6-chloro-3,4-dihydro-2H-benzo[b][1,4]oxazine-2-carboxamide

The methodologies described in Example 71B substituting Example 85J for Example 71A without purification gave the title compound. MS (ESI⁺) m/z 352 (M+H)⁺.

### Example 85L: (2S)-6-chloro-N-{(3S)-4-[2-(4-chloro-3-fluorophenoxy)acetamido]-3-hydroxybicyclo[2.2.2]octan-1-yl}-3,4-dihydro-2H-1,4-benzoxazine-2-carboxamide

The methodologies described in Example 14 substituting 2-(4-chloro-3-fluorophenoxy)acetic acid for 6-chloro-3,4-dihydro-2H-benzo[b][1,4]oxazine-2-carboxylic acid and substituting Example 85K for Example 23B gave the title compound. MS (ESI⁺) m/z 538 (M+H)⁺.

### Example 85M: tert-butyl [(2S)-6-chloro-2-({(3S)-4-[2-(4-chloro-3-fluorophenoxy)acetamido]-3-hydroxybicyclo[2.2.2]octan-1-yl}carbamoyl)-2,3-dihydro-4H-1,4-benzoxazin-4-yl]acetate

The methodologies described in Example 36 with the following modifications: (1) substituting tert-butyl bromoacetate for 2-bromoethanol, (2) substituting Example 85L for the product of Example 14, (3) stirring for 2 days instead of 5, and (4) with the addition of NaI (0.5 equivalence), gave the title compound. ¹H NMR 1H NMR (400 MHz, DMSO-d₆) δ ppm 7.48 (t, J=8.9 Hz, 1H), 7.36 (s, 1H), 7.26 (s, 1H), 7.05 (dd, J=11.4, 2.8 Hz, 1H), 6.83 (dd, J=8.7, 3.1 Hz, 2H), 6.63-6.53 (m, 2H), 5.08 (d, J=4.4 Hz, 1H), 4.52-4.45 (m, 1H), 4.46 (s, 2H), 4.15 (d, J=18.1 Hz, 1H), 4.05 (s, 1H), 4.03 (s, 1H), 3.48 (dd, J=11.9, 2.7 Hz, 1H), 2.27 (t, J=11.4 Hz, 1H), 2.07 (s, 1H), 2.06 (t, J=9.3 Hz, 1H), 1.93 (d, J=10.7 Hz, 2H), 1.81 (dt, J=23.1, 12.2 Hz, 6H), 1.43 (d, J=17.1 Hz, 1H), 1.38 (s, 9H); MS (ESI⁺) m/z 596 (M-Boc+H)⁺.

### Example 86: tert-butyl [6-chloro-2-({(3S)-4-[2-(4-chloro-3-fluorophenoxy)acetamido]-3-hydroxybicyclo[2.2.2]octan-1-yl}carbamoyl)-2,3-dihydro-4H-1,4-benzoxazin-4-yl]acetate (Compound 185)

The methodologies described in Example 85 substituting 6-chloro-3,4-dihydro-2H-benzo[b][1,4]oxazine-2-carboxylic acid for Example 851 in the reaction sequence provided tert-butyl {(2S)-4-[(6-chloro-3,4-dihydro-2H-1,4-benzoxazine-2-carbonyl)amino]-2-hydroxybicyclo[2.2.2]octan-1-yl}carbamate, which was carried forward in the reaction sequence to give the title compound. The spectral data match Example 85.

### Example 87: 6-chloro-N-{3-[2-(4-chloro-3-fluorophenoxy)acetamido]bicyclo[1.1.1]pentan-1-yl}-4-oxo-3,4-dihydro-2H-1-benzopyran-2-carboxamide (Compound 186)

The methodologies described in Example 14 substituting 6-chloro-4-oxochroman-2-carboxylic acid for 6-chloro-3,4-dihydro-2H-benzo[b][1,4]oxazine-2-carboxylic acid gave the title compound. ¹H NMR (400 MHz, DMSO-d₆) δ ppm 8.95 (s, 1H), 8.73 (s, 1H), 7.68-7.60 (m, 2H), 7.49 (t, J=8.9 Hz, 1H), 7.17 (d, J=8.5 Hz, 1H), 7.07 (dd, J=11.3, 2.9 Hz, 1H), 6.85 (ddd, J=9.0, 2.9, 1.2 Hz, 1H), 5.09 (t, J=7.1 Hz, 1H), 4.47 (s, 2H), 2.95 (d, J=7.1 Hz, 2H), 2.26 (s, 6H); MS (ESI⁺) m/z 493 (M+H)⁺.

### Example 88: 6-chloro-N-{3-[2-(4-chloro-3-fluorophenoxy)acetamido]bicyclo[1.1.1]pentan-1-yl}-4-(2-methoxy-2-methylpropyl)-3,4-dihydro-2H-1,4-benzoxazine-2-carboxamide (Compound 187)

To a solution of Example 14 (0.090 g, 0.187 mmol) in methanol (1.2 mL) was added 2-methoxy-2-methylpropanal (0.029 g, 0.281 mmol) and zinc chloride (0.141 mL, 0.281 mmol, 1.9 M in 2-methyltetrahydrofuran). After stirring at ambient temperature for 30 minutes, sodium cyanoborohydride (0.018 g, 0.281 mmol) was added, and this mixture was allowed to stir at ambient temperature for 15 minutes, then 50° C. overnight. More methoxy-2-methylpropanal (0.029 g, 0.281 mmol) and sodium cyanoborohydride (0.018 g, 0.281 mmol) were added to the reaction mixture and stirring was continued at 50° C. for 24 hours. Then the reaction mixture was cooled to ambient temperature, diluted with N,N-dimethylformamide/water (1.2 mL, 3:1) and purified by preparative HPLC [Waters XBridge™ C18 5 μm OBD column, 30×100 mm, flow rate 40 mL/minute, 5-100% gradient of acetonitrile in buffer (0.025 M aqueous ammonium bicarbonate, adjusted to pH 10 with ammonium hydroxide)] to give the title compound (0.007 g, 0.012 mmol, 7% yield). ¹H NMR (501 MHz, DMSO-d₆) δ ppm 8.71 (s, 1H), 8.67 (s, 1H), 7.49 (t, J=8.9 Hz, 1H), 7.07 (dd, J=11.4, 2.8 Hz, 1H), 6.89 (d, J=2.4 Hz, 1H), 6.85 (ddd, J=9.0, 2.9, 1.2 Hz, 1H), 6.77 (d, J=8.5 Hz, 1H), 6.54 (dd, J=8.4, 2.4 Hz, 1H), 4.47 (s, 2H), 4.44 (dd, J=7.2, 2.8 Hz, 1H), 3.55 (dd, J=12.7, 2.9 Hz, 1H), 3.27 (d, J=15.2 Hz, 1H), 3.17 (d, J=15.1 Hz, 1H), 3.13 (s, 3H), 2.25 (s, 6H), 1.12 (d, J=5.4 Hz, 6H); MS (ESI⁺) m/z 566 (M+H)⁺.

### Example 89: 6-chloro-N-{3-[2-(4-chloro-3-fluorophenoxy)acetamido]bicyclo[1.1.1]pentan-1-yl}-4-[(2-hydroxyethyl)amino]-3,4-dihydro-2H-1-benzopyran-2-carboxamide (Compound 188)

To a solution of Example 87 (0.070 g, 0.142 mmol) in methanol (1 mL) was added 2-((trimethylsilyl)oxy)ethanamine (0.028 g, 0.21 mmol) and zinc chloride (powdered, 0.029 g, 0.213 mmol). After stirring at ambient temperature for 30 minutes, sodium cyanoborohydride (0.013 g, 0.213 mmol) was added, and this mixture was allowed to stir at ambient temperature for 15 minutes, then 50° C. overnight. Then the reaction mixture was cooled to ambient temperature, diluted with N,N-dimethylformamide/water (1.2 mL, 3:1) and purified by preparative HPLC (Waters XBridge™ C18 5 μm OBD column, 30×100 mm, flow rate 40 mL/minute, 5-100% gradient of acetonitrile in 0.1% trifluoroacetic acid/water) to give the title compound (0.05 g, 0.093 mmol, 65% yield). ¹H NMR (500 MHz, DMSO-d₆) δ ppm 9.17 (s, 2H), 9.04 (s, 2H), 8.94 (s, 2H), 8.87 (s, 1H), 8.76 (d, J=4.0 Hz, 3H), 7.72 (d, J=2.5 Hz, 2H), 7.61 (d, J=2.6 Hz, 1H), 7.50 (t, J=8.9 Hz, 3H), 7.41 (ddd, J=25.1, 8.9, 2.6 Hz, 3H), 7.10-7.01 (m, 7H), 6.86 (dd, J=9.0, 2.8 Hz, 3H), 4.86 (d, J=8.2 Hz, 2H), 4.74 (dd, J=11.9, 2.8 Hz, 1H), 4.62 (dd, J=11.3, 2.3 Hz, 2H), 4.53 (s, 1H), 4.49 (d, J=1.6 Hz, 6H), 3.74-3.67 (m, 6H), 3.20-3.07 (m, 7H), 2.98 (m, 3H), 2.65-2.56 (m, 2H), 2.30 (s, 12H), 2.29 (s, 6H), 2.20-2.08 (m, 2H), 2.03 (dt, J=13.3, 11.0 Hz, 2H); MS (ESI⁺) m/z 538 (M+H)⁺.

### Example 90: methyl {[6-chloro-2-({3-[2-(4-chloro-3-fluorophenoxy)acetamido]bicyclo[1.1.1]pentan-1-yl}carbamoyl)-3,4-dihydro-2H-1-benzopyran-4-yl]amino}acetate (Compound 189)

The methodologies described in Example 89 substituting methyl 2-aminoacetate hydrochloride for 2-((trimethylsilyl)oxy)ethanamine gave the title compound. ¹H NMR 1H NMR (400 MHz, DMSO-d₆) δ ppm 8.95 (s, 1H), 8.84 (s, 1H), 8.76 (d, J=2.3 Hz, 2H), 7.72 (d, J=2.5 Hz, 1H), 7.62 (d, J=2.6 Hz, 1H), 7.50 (t, J=8.8 Hz, 2H), 7.40 (ddd, J=11.8, 8.9, 2.5 Hz, 2H), 7.10-7.01 (m, 4H), 6.86 (dd, J=8.9, 2.8 Hz, 2H), 4.82 (dd, J=10.1, 6.5 Hz, 1H), 4.75 (dd, J=12.0, 2.6 Hz, 1H), 4.61 (dd, J=11.0, 2.4 Hz, 1H), 4.49 (s, 4H), 4.21 (d, J=16.9 Hz, 1H), 4.11 (d, J=10.5 Hz, 3H), 3.77 (d, J=1.7 Hz, 6H), 2.64 (ddd, J=13.5, 6.6, 2.4 Hz, 1H), 2.56 (d, J=15.5 Hz, 1H), 2.30 (d, J=2.7 Hz, 12H), 2.16-2.08 (m, 1H), 2.07 (s, 2H), 2.03 (dd, J=12.3, 9.5 Hz, 1H); MS (ESI⁺) m/z 566 (M+H)⁺.

### Example 91: rac-(2R,4R)-6-chloro-N-{3-[2-(4-chloro-3-fluorophenoxy)acetamido]bicyclo[1.1.1]pentan-1-yl}-4-hydroxy-3,4-dihydro-2H-1-benzopyran-2-carboxamide (Compound 190)

To a solution of Example 87 (1.579 g, 3.20 mmol) in methanol (21 mL) was added zinc chloride (powdered, 654 mg, 4.8 mmol). After stirring at 50° C. for 5 minutes, sodium cyanoborohydride (302 mg, 4.8 mmol) was added, and this mixture was allowed to stir at 50° C. Over the subsequent 6 days, additional aliquots of zinc chloride (powdered, 327 mg, 2.4 mmol) and sodium cyanoborohydride (285 mg, 4.53 mmol) were added each day. Then the reaction mixture was cooled to ambient temperature, concentrated, diluted with N,N-dimethylformamide/water (1.2 mL, 3:1) and purified by preparative HPLC (Waters XBridge™ C18 5 μm OBD column, 30×100 mm, flow rate 40 mL/minute, 5-100% gradient of acetonitrile in 0.1% trifluoroacetic acid/water) to give the title compound. The corresponding trans isomer was also determined to be present in minor amount (cis:trans=12.5:1). ¹H NMR (501 MHz, DMSO-d₆) δ ppm 8.73 (s, 1H), 8.69 (s, 1H), 7.50 (t, J=8.9 Hz, 1H), 7.38 (dd, J=2.8, 1.0 Hz, 1H), 7.24-7.17 (m, 1H), 7.14-7.04 (m, 1H), 6.94-6.83 (m, 2H), 4.81 (dd, J=10.7, 5.9 Hz, 1H), 4.60 (dd, J=12.0, 2.3 Hz, 1H), 4.48 (s, 2H), 2.35 (ddd, J=12.9, 5.9, 2.3 Hz, 1H), 2.29 (s, 6H), 2.07 (s, 1H), 1.70 (td, J=12.5, 10.8 Hz, 1H); MS (ESI⁺) m/z 477 (M−H₂O+H)⁺.

### Example 92: (2S)-6-chloro-N-{3-[2-(4-chloro-3-fluorophenoxy)acetamido]bicyclo[1.1.1]pentan-1-yl}-4-(2-methoxy-2-methylpropyl)-3,4-dihydro-2H-1,4-benzoxazine-2-carboxamide (Compound 191)

The methodologies described in Example 88 provided the title compound using the following modifications: (1) substituting Example 4 for Example 14, (2) substituting powdered zinc chloride for zinc chloride (1.9 M in 2-methyltetrahydrofuran), and (3) halting the reaction after 24 hours, although incomplete conversion. The spectral data match Example 88.

### Example 93: {[6-chloro-2-({3-[2-(4-chloro-3-fluorophenoxy)acetamido]bicyclo[1.1.1]pentan-1-yl}carbamoyl)-3,4-dihydro-2H-1-benzopyran-4-yl]amino}acetic acid (Compound 192)

To a solution of the product of Example 90 (0.025 g, 0.044 mmol) in tetrahydrofuran (0.036 mL) was added LiOH (0.02 mL, 1 N in water), and the reaction mixture stirred at ambient temperature overnight. Then to the mixture was added more LiOH (powder, 0.0031 g, 0.13 mmol), and the reaction mixture was stirred for another 2.5 hours and then was concentrated. The residue was diluted with N,N-dimethylformamide/water (2 mL, 3:1) and purified by preparative HPLC (Waters XBridge™ C18 5 μm OBD column, 30×100 mm, flow rate 40 mL/minute, 5-100% gradient of acetonitrile in 0.1% trifluoroacetic acid/water) to yield the title compound (0.015 g, 62%) as a mixture of diastereomers (dr 2:1). ¹H NMR (400 MHz, DMSO-d₆) δ ppm 8.92 (s, 2H), 8.84 (s, 1H), 8.75 (d, J=2.0 Hz, 3H), 7.72 (d, J=2.5 Hz, 2H), 7.62 (d, J=2.6 Hz, 1H), 7.50 (t, J=8.9 Hz, 3H), 7.40 (ddd, J=12.4, 8.8, 2.6 Hz, 3H), 7.10-7.01 (m, 6H), 6.86 (ddd, J=9.0, 3.0, 1.2 Hz, 3H), 4.84-4.75 (m, 3H), 4.73 (d, J=2.0 Hz, 1H), 4.61 (dd, J=11.0, 2.3 Hz, 2H), 4.49 (s, 6H), 4.50-4.44 (m, 1H), 4.08 (d, J=16.8 Hz, 1H), 3.95 (m, 4H), 3.95 (d, J=16.8 Hz, 1H), 2.68-2.52 (m, 3H), 2.30 (s, 12H), 2.29 (s, 6H), 2.16-2.05 (m, 1H), 2.09-2.02 (m, 1H), 2.04-1.96 (m, 1H); MS (ESI⁺) m/z 553 (M+H)⁺.

### Example 94: (2S)-6-chloro-N-{3-[2-(4-chloro-3-fluorophenoxy)acetamido]bicyclo[1.1.1]pentan-1-yl}-4-(2-methylprop-2-en-1-yl)-3,4-dihydro-2H-1,4-benzoxazine-2-carboxamide (Compound 193)

To a solution of the product of Example 92 (0.020 g, 0.035 mmol) in CH₂Cl₂ (0.35 mL) was added boron tribromide (0.07 mL, 0.07 mmol, 1 M in CH₂Cl₂) in an ice bath. The reaction mixture was allowed to warm to ambient temperature in the ice bath for 2 hours and then was partitioned between water (1 mL) and CH₂Cl₂ (3 mL), and the layers were separated. The aqueous layer was extracted with CH₂Cl₂ (2×10 mL), and the combined organic layers were dried (Na₂SO₄), filtered, and concentrated. The residue was diluted with N,N-dimethylformamide/water (1.2 mL, 3:1) and purified by preparative HPLC (Waters XBridge™ C18 5 μm OBD column, 30×100 mm, flow rate 40 mL/minute, 5-100% gradient of acetonitrile in 0.1% trifluoroacetic acid/water) to yield the title compound (0.005 g, 0.009 mmol, 27% yield). ¹H NMR (501 MHz, DMSO-d₆) δ ppm 8.71 (s, 1H), 8.69 (s, 1H), 7.49 (t, J=8.9 Hz, 1H), 7.07 (dd, J=11.4, 2.9 Hz, 1H), 6.88-6.83 (m, 1H), 6.80 (d, J=8.4 Hz, 1H), 6.62 (d, J=2.4 Hz, 1H), 6.57 (dd, J=8.5, 2.4 Hz, 1H), 4.87 (t, J=1.6 Hz, 1H), 4.81 (s, 1H), 4.57 (dd, J=6.8, 3.0 Hz, 1H), 4.47 (s, 2H), 3.83 (d, J=16.6 Hz, 1H), 3.72 (d, J=16.6 Hz, 1H), 3.29 (dd, J=12.3, 6.8 Hz, 1H), 2.25 (s, 6H), 1.67 (s, 3H); MS (ESI⁺) m/z 534 (M+H)⁺.

### Example 95: tert-butyl [(2R)-6-chloro-2-({(3S)-4-[2-(4-chloro-3-fluorophenoxy)acetamido]-3-hydroxybicyclo[2.2.2]octan-1-yl}carbamoyl)-2,3-dihydro-4H-1,4-benzoxazin-4-yl]acetate (Compound 194)

### Example 95A: (S)-4-chloro-2-((3-chloro-2-hydroxypropyl)amino)phenol

To a solution of 2-amino-4-chlorophenol (25 g, 174 mmol) in ethanol (250 mL) and water (2.5 mL) was added (S)-2-(chloromethyl)oxirane (16.1 g, 174 mmol), and the solution was stirred for 12 hours at 60° C. Then the reaction mixture was concentrated, and the crude residue was chromatographed on silica gel (ethyl acetate/petroleum ether 1:5) to give the title compound (80 g, 305 mmol, 73% yield). ¹H NMR (400 MHz, CDCl₃) δ ppm 3.19-3.39 (m, 2H), 3.61-3.73 (m, 2H), 4.12-4.19 (m, 1H), 6.59 (s, 1H), 6.62 (br s, 1H), 6.62-6.66 (m, 1H).

### Example 95B: (R)-(6-chloro-3,4-dihydro-2H-benzo[b][1,4]oxazin-2-yl)methanol

To a solution of Example 95A (40 g, 152 mmol) in ethanol (400 mL) was added K₂CO₃ (12.6 g, 91 mmol), and the solution was stirred for 12 hours at 90° C. The reaction solution was filtered and concentrated under reduced pressure. The mixture was diluted with water (1000 mL) and extracted with ethyl acetate (3×1000 mL). The combined organic layers were washed with brine, dried over Na₂SO₄, filtered, and concentrated under reduced pressure. The residue was chromatographed on silica gel (ethyl acetate/petroleum ether, 1:4) to give the title compound (30 g, 135 mmol, 89% yield). ¹H NMR (400 MHz, CDCl₃) δ ppm 3.28-3.42 (m, 2H) 3.84 (qd, J=11.72, 4.96 Hz, 2H) 4.18-4.25 (m, 1H) 6.58-6.64 (m, 2H) 6.71-6.75 (m, 1H) 6.73 (d, J=8.60 Hz, 1H).

### Example 95C: (R)-tert-butyl 2-(((tert-butoxycarbonyl)oxy)methyl)-6-chloro-2H-benzo[b][1,4]oxazine-4(3H)-carboxylate

To a solution of Example 95B (16 g, 72 mmol) in dichloromethane (160 mL) was added triethylamine (25.1 mL, 180 mmol), di-tert-butyl dicarbonate (67 mL, 289 mmol) and 4-(dimethylamino)pyridine (0.88 g, 7.2 mmol). The solution stirred for 2 hours at 20° C. Then water (500 mL) was added to the mixture, and the aqueous mixture was extracted with dichloromethane (2×500 mL). The combined organic layers were washed with brine (300 mL), dried over Na₂SO₄, filtered, and concentrated. The residue was purified by silica gel chromatography eluted with petroleum ether:ethyl acetate (10:1) to give the title compound (40 g, 90 mmol, 62% yield). ¹H NMR (400 MHz, DMSO, d6) δ ppm 1.42 (s, 9H) 3.07 (br dd, J=11.25, 7.28 Hz, 1H) 4.11-4.29 (m, 3H) 6.12 (br s, 1H) 6.41-6.53 (m, 1H) 6.60 (br d, J=2.20 Hz, 1H) 6.67 (br d, J=8.38 Hz, 1H).

### Example 95D: (R)-tert-butyl 6-chloro-2-(hydroxymethyl)-2H-benzo[b][1,4]oxazine-4(3H)-carboxylate

To a solution of Example 95C (36 g, 81 mmol) in methanol (360 mL) and tetrahydrofuran (360 mL) was added a solution of NaOH (6.48 g, 162 mmol) in water (360 mL) at 20° C., and the mixture was stirred at 20° C. for 12 hours. The mixture was concentrated and extracted with ethyl acetate (3×500 mL). The combined organic layers were washed with brine (300 mL), dried over Na₂SO₄, and concentrated to give the title compound (20 g, 60 mmol, 67% yield). ¹H NMR (400 MHz, CDCl₃) δ ppm 1.56 (s, 9H) 3.57 (dd, J=13.69, 7.83 Hz, 1H) 3.81 (br d, J=3.91 Hz, 2H) 4.07 (dd, J=13.69, 2.45 Hz, 1H) 4.25 (dtd, J=7.58, 5.01, 5.01, 2.93 Hz, 1H) 6.83 (d, J=8.31 Hz, 1H) 6.94-6.98 (m, 1H) 7.80 (br s, 1H).

### Example 95E: (R)-4-(tert-butoxycarbonyl)-6-chloro-3,4-dihydro-2H-benzo[b][1,4]oxazine-2-carboxylic Acid

To a solution of Example 95D (16 g, 48 mmol) in dichloromethane (160 mL) was added N-methylmorpholine-N-oxide (NMO, 33.8 g, 288 mmol) at 0° C. and tetrapropylammonium perruthenate (TPAP, 3.38 g, 9.61 mmol) at 0° C. The solution was stirred for 2 hours at 20° C. and then was concentrated. The residue was purified by preparative HPLC (Waters XBridge™ C18 5 μm OBD column, 30×100 mm, flow rate 40 mL/minute, 5-100% gradient of methanol in 0.1% trifluoroacetic acid/water) to give the title compound (20 g, 57 mmol, 90% yield). ¹H NMR (400 MHz, CDCl₃) δ ppm 1.50-1.55 (m, 9H) 3.87 (dd, J=13.89, 3.31 Hz, 1H) 4.29 (dd, J=13.78, 4.74 Hz, 1H) 4.87-4.92 (m, 1H) 6.89-6.96 (m, 1H) 6.97-7.03 (m, 1H) 7.77 (br s, 1H) 8.02 (br s, 2H).

### Example 95F: (R)-6-chloro-3,4-dihydro-2H-benzo[b][1,4]oxazine-2-carboxylic Acid hydrochloride

To a solution of Example 95E (10 g, 29 mmol) in dichloromethane (100 mL) was added HCl (28.7 mL, 115 mmol, 4 molar in ethyl acetate) at 0° C. The solution stirred for 12 hours at 20° C., and then the solid was collected by suction filtration and was dried to give the title compound (13.1 g, 57.8 mmol, 101% yield). ¹H NMR (400 MHz, DMSO-d₆) δ ppm 3.34-3.44 (m, 2H) 4.82 (t, J=3.75 Hz, 1H) 6.50 (dd, J=8.49, 2.54 Hz, 1H) 6.58 (d, J=2.43 Hz, 1H) 6.71 (d, J=8.38 Hz, 1H); MS (ESI⁻) m/z 212 (M−H)⁻.

### Example 95G: tert-butyl ((S)-4-((R)-6-chloro-3,4-dihydro-2H-benzo[b][1,4]oxazine-2-carboxamido)-2-hydroxybicyclo[2.2.2]octan-1-yl)carbamate

The methodologies described in Example 14 substituting Example 95F for 6-chloro-3,4-dihydro-2H-benzo[b][1,4]oxazine-2-carboxylic acid and substituting 85C for Example 23B gave the title compound. MS (ESI⁺) m/z 452 (M+H)⁺.

### Example 95H: (R)—N—((S)-4-amino-3-hydroxybicyclo[2.2.2]octan-1-yl)-6-chloro-3,4-dihydro-2H-benzo[b][1,4]oxazine-2-carboxamide

The methodologies described in Example 71B substituting Example 95G for Example 71A without purification gave the title compound. MS (ESI⁺) m/z 352 (M+H)⁺.

### Example 95I: (2R)-6-chloro-N-{(3S)-4-[2-(4-chloro-3-fluorophenoxy)acetamido]-3-hydroxybicyclo[2.2.2]octan-1-yl}-3,4-dihydro-2H-1,4-benzoxazine-2-carboxamide

The methodologies described in Example 14 substituting 2-(4-chloro-3-fluorophenoxy)acetic acid for 6-chloro-3,4-dihydro-2H-benzo[b][1,4]oxazine-2-carboxylic acid and substituting Example 95H for Example 23B gave the title compound. MS (ESI⁺) m/z 538 (M+H)⁺.

### Example 95J. tert-butyl [(2R)-6-chloro-2-({(3S)-4-[2-(4-chloro-3-fluorophenoxy)acetamido]-3-hydroxybicyclo[2.2.2]octan-1-yl}carbamoyl)-2,3-dihydro-4H-1,4-benzoxazin-4-yl]acetate

The methodologies described in Example 36 with the following modifications: (1) substituting tert-butyl bromoacetate for 2-bromoethanol, (2) substituting Example 951 for the product of Example 14, (3) stirring for 2 days instead of 5, and (4) with the addition of NaI (0.5 equivalence), gave the title compound. ¹H NMR (400 MHz, DMSO-d₆) δ ppm 7.48 (t, J=9.0 Hz, 1H), 7.37 (s, 1H), 7.26 (s, 1H), 7.05 (dd, J=11.5, 2.9 Hz, 1H), 6.82 (d, J=8.4 Hz, 2H), 6.63-6.53 (m, 2H), 5.08 (m, 1H), 4.46 (m, 3H), 4.15 (d, J=18.3 Hz, 1H), 4.05 (m, 1H), 1.93 (m, 2H), 1.80 (m, 7H), 1.38 (s, 9H); MS (ESI⁺) m/z 596 (M-C(O)OC(CH₃)₃+H)⁺.

### Example 96: (2S,4S)-6-chloro-N-{3-[2-(4-chloro-3-fluorophenoxy)acetamido]bicyclo[1.1.1]pentan-1-yl}-4-hydroxy-3,4-dihydro-2H-1-benzopyran-2-carboxamide (Compound 195)

Example 91 was purified by chiral SFC (supercritical fluid chromatography) using a Whelk-O®1 column eluting with 40% CH₃OH with 0.1% diethylamine in CO₂ with a flow rate of 80 g/minute and back pressure of 120 bar to give the title compound (first enantiomer eluted out of the column, 0.011 g, 0.022 mmol, 29% yield). ¹H NMR (400 MHz, DMSO-d₆) δ ppm 8.72 (s, 1H), 8.68 (s, 1H), 7.50 (t, J=8.9 Hz, 1H), 7.38 (dd, J=2.7, 1.0 Hz, 1H), 7.20 (ddd, J=8.7, 2.7, 0.6 Hz, 1H), 7.08 (dd, J=11.4, 2.9 Hz, 1H), 6.92-6.82 (m, 2H), 5.70 (d, J=6.3 Hz, 1H), 4.81 (dt, J=11.6, 6.0 Hz, 1H), 4.60 (dd, J=12.0, 2.3 Hz, 1H), 4.48 (s, 2H), 2.35 (ddd, J=12.9, 5.9, 2.3 Hz, 1H), 2.29 (s, 6H), 1.76-1.63 (m, 1H); MS (ESI⁺) m/z 477 (M−H₂O+H)⁺.

### Example 97: (2R,4R)-6-chloro-N-{3-[2-(4-chloro-3-fluorophenoxy)acetamido]bicyclo[1.1.1]pentan-1-yl}-4-hydroxy-3,4-dihydro-2H-1-benzopyran-2-carboxamide (Compound 196)

Example 91 was purified by chiral SFC (supercritical fluid chromatography) using a Whelk-O®1 column eluting with 40% CH₃OH with 0.1% diethylamine in CO₂ with a flow rate of 80 g/minute and back pressure of 120 bar to give the title compound (fourth enantiomer eluted out of the column, 0.017 g, 0.010 mmol, 45% yield). ¹H NMR (400 MHz, DMSO-d₆) δ ppm 8.72 (s, 1H), 8.68 (s, 1H), 7.50 (t, J=8.9 Hz, 1H), 7.38 (dd, J=2.8, 1.0 Hz, 1H), 7.20 (dd, J=8.7, 2.7 Hz, 1H), 7.08 (dd, J=11.4, 2.8 Hz, 1H), 6.91-6.82 (m, 2H), 5.70 (d, J=6.4 Hz, 1H), 4.80 (dt, J=11.4, 6.0 Hz, 1H), 4.59 (dd, J=12.0, 2.2 Hz, 1H), 4.48 (s, 2H), 2.40-2.30 (m, 1H), 2.28 (s, 6H), 1.69 (td, J=12.5, 10.8 Hz, 1H); MS (ESI⁺) m/z 477 (M−H₂O+H)⁺.

### Alternative Synthesis of Example 97: (2R,4R)-6-chloro-N-{3-[2-(4-chloro-3-fluorophenoxy)acetamido]bicyclo[1.1.1]pentan-1-yl}-4-hydroxy-3,4-dihydro-2H-1-benzopyran-2-carboxamide (Compound 196)

To a suspension of the product of Example 124 (28 mg, 0.057 mmol) in methanol (1 mL) was added sodium borohydride (4.29 mg, 0.114 mmol) at 0° C. The ice bath was then removed and the reaction mixture was slowly warmed to ambient temperature over 10 minutes. After stirring at ambient temperature for 10 minutes, a saturated aqueous solution of NH₄Cl (0.2 mL) was added, and the resulting solution was stirred for 5 minutes and then was partitioned between ethyl acetate (2×10 mL) and water (10 mL). The combined organic phases were dried over sodium sulfate, filtered, concentrated in vacuo. The residue was dissolved in N, N-dimethylformamide (3 mL), filtered through a glass microfiber frit, and purified by preparative HPLC [YMC TriArt™ C18 Hybrid 5 μm column, 50×100 mm, flow rate 140 mL/minute, 5-100% gradient of acetonitrile in buffer (0.025 M aqueous ammonium bicarbonate, adjusted to pH 10 with ammonium hydroxide)] to give the title compound (26 mg, 0.052 mmol, 92% yield). ¹H NMR (501 MHz, DMSO-d₆) δ ppm 8.72 (s, 1H), 8.67 (s, 1H), 7.50 (t, J=8.9 Hz, 1H), 7.38 (dd, J=2.6, 1.0 Hz, 1H), 7.20 (ddd, J=8.7, 2.7, 0.7 Hz, 1H), 7.08 (dd, J=11.4, 2.9 Hz, 1H), 6.88 (d, J=8.8 Hz, 1H), 6.87-6.84 (m, 1H), 5.69 (s, 1H), 4.82-4.78 (m, 1H), 4.59 (dd, J=12.0, 2.3 Hz, 1H), 4.48 (s, 2H), 2.35 (ddd, J=12.9, 5.9, 2.4 Hz, 1H), 2.28 (s, 6H), 1.75-1.64 (m, 1H); MS (APCI⁺) m/z 477 (M−H₂O+H)⁺.

### Example 98: (2R,4S)-6-chloro-N-{3-[2-(4-chloro-3-fluorophenoxy)acetamido]bicyclo[1.1.1]pentan-1-yl}-4-hydroxy-3,4-dihydro-2H-1-benzopyran-2-carboxamide (Compound 197)

Example 91 was purified by chiral SFC (supercritical fluid chromatography) using a Whelk-O®1 column eluting with 40% CH₃OH with 0.1% diethylamine in CO₂ with a flow rate of 80 g/minute and back pressure of 120 bar to give the title compound (third enantiomer eluted out of the column, 0.003 g, 0.006 mmol, 8% yield). ¹H NMR (501 MHz, DMSO-d₆) δ ppm 8.73 (s, 1H), 8.72 (s, 1H), 7.50 (t, J=8.9 Hz, 1H), 7.31 (d, J=2.6 Hz, 1H), 7.25 (dd, J=8.8, 2.7 Hz, 1H), 7.08 (dd, J=11.4, 2.9 Hz, 1H), 6.93 (d, J=8.8 Hz, 1H), 6.86 (ddd, J=9.0, 2.9, 1.2 Hz, 1H), 5.61 (d, J=4.7 Hz, 1H), 4.58 (q, J=4.0 Hz, 1H), 4.55 (dd, J=10.9, 2.7 Hz, 1H), 4.48 (s, 2H), 2.28 (s, 6H), 2.09 (ddd, J=13.9, 3.9, 2.8 Hz, 1H), 1.89 (ddd, J=14.3, 11.0, 3.7 Hz, 1H); MS (ESI⁺) m/z 477 (M−H₂O+H)⁺.

### Example 99: (2S,4R)-6-chloro-N-{3-[2-(4-chloro-3-fluorophenoxy)acetamido]bicyclo[1.1.1]pentan-1-yl}-4-hydroxy-3,4-dihydro-2H-1-benzopyran-2-carboxamide (Compound 198)

Example 91 was purified by chiral SFC (supercritical fluid chromatography) using a Whelk-O®1 column eluting with 40% CH₃OH with 0.1% diethylamine in CO₂ with a flow rate of 80 g/minute and back pressure of 120 bar to give the title compound (second enantiomer eluted out of the column, 0.002 g, 0.004 mmol, 5% yield). This title compound contained Example 96 as the major product in a mixture of diastereomers (dr 5:1, Example 96:99). ¹H NMR (400 MHz, DMSO-d₆, dr 5:1) δ ppm 8.74 (s, 1H), 8.72 (s, 1H), 8.68 (s, 0.2H), 7.50 (t, J=8.9 Hz, 1H), 7.38 (d, J=2.2 Hz, 0.2H), 7.31 (d, J=2.7 Hz, 1H), 7.25 (dd, J=8.8, 2.7 Hz, 1H), 7.20 (dd, J=8.7, 2.6 Hz, 0.2H), 7.08 (dd, J=11.4, 2.8 Hz, 1H), 6.91 (dd, J=18.7, 8.3 Hz, 1H), 6.89-6.82 (m, 1H), 5.70 (d, J=6.3 Hz, 0.2H), 5.61 (d, J=4.7 Hz, 1H), 4.80 (dt, J=11.5, 6.2 Hz, 0.2H), 4.59-4.52 (m, 2H), 4.48 (s, 2H), 2.28 (d, J=1.9 Hz, 6H), 2.09 (dt, J=13.8, 3.4 Hz, 1H), 1.89 (ddd, J=14.1, 10.9, 3.7 Hz, 1H); MS (ESI⁺) m/z 477 (M−H₂O+H)⁺.

### Alternative synthesis of Example 99: (2S,4R)-6-chloro-N-{3-[2-(4-chloro-3-fluorophenoxy)acetamido]bicyclo[1.1.1]pentan-1-yl}-4-hydroxy-3,4-dihydro-2H-1-benzopyran-2-carboxamide (Compound 198)

To a mixture of Example 270N (18 mg, 0.080 mmol), Example 23B (23 mg, 0.080 mmol) and N,N-diisopropylethylamine (0.098 mL, 0.560 mmol) in N,N-dimethylformamide (1 mL) was added 1-propanephosphonic anhydride (50% in N,N-dimethylformamide) (0.054 mL, 0.092 mmol), and the resulting mixture was stirred at room temperature for 1 hour. The mixture was partitioned between water (10 mL) and dichloromethane (2×10 mL), and the combined organic extracts were dried over sodium sulfate and concentrated in vacuo. The crude product was purified by HPLC [Waters XBridge™ C18 5 μm OBD column, 30×100 mm, flow rate 40 mL/minute, 5-100% gradient of acetonitrile in buffer (0.025 M aqueous ammonium bicarbonate, adjusted to pH 10 with ammonium hydroxide)] to give the title compound (33 mg, 0.067 mmol, 83% yield). ¹H NMR (DMSO-d₆) δ ppm 8.73 (s, 1H), 8.71 (s, 1H), 7.50 (t, J=8.9 Hz, 1H), 7.31 (d, J=2.6 Hz, 1H), 7.25 (dd, J=8.7, 2.7 Hz, 1H), 7.08 (dd, J=11.4, 2.9 Hz, 1H), 6.93 (d, J=8.7 Hz, 1H), 6.85 (ddd, J=8.9, 2.8, 1.2 Hz, 1H), 5.60 (d, J=4.6 Hz, 1H), 4.61-4.53 (m, 2H), 4.48 (s, 2H), 2.28 (d, J=1.9 Hz, 6H), 2.12-2.05 (m, 1H), 1.89 (ddd, J=14.2, 11.0, 3.7 Hz, 1H); MS (APCI) m/z 495 (M+H)⁺.

### Example 100: 6-chloro-N-{(3S)-4-[2-(4-chloro-3-fluorophenoxy)acetamido]-3-hydroxybicyclo[2.2.2]octan-1-yl}-4-hydroxy-3,4-dihydro-2H-1-benzopyran-2-carboxamide (Compound 199)

### Example 100A: tert-butyl ((2S)-4-(6-chloro-4-oxochroman-2-carboxamido)-2-hydroxybicyclo[2.2.2]octan-1-yl)carbamate

The methodologies described in Example 14 substituting 6-chloro-4-oxochroman-2-carboxylic acid for 6-chloro-3,4-dihydro-2H-benzo[b][1,4]oxazine-2-carboxylic acid and substituting Example 85C for Example 23B gave the title compound. MS (ESI⁺) m/z 465 (M+H)⁺.

### Example 100B: N—((S)-4-amino-3-hydroxybicyclo[2.2.2]octan-1-yl)-6-chloro-4-oxochroman-2-carboxamide

The methodologies described in Example 71B substituting Example 100A for Example 71A without purification gave the title compound. MS (ESI⁺) m/z 365 (M+H)⁺.

### Example 10C: 6-chloro-N-{(3S)-4-[2-(4-chloro-3-fluorophenoxy)acetamido]-3-hydroxybicyclo[2.2.2]octan-1-yl}-4-oxo-3,4-dihydro-2H-1-benzopyran-2-carboxamide (Compound 199C)

The methodologies described in Example 14 substituting 2-(4-chloro-3-fluorophenoxy)acetic acid for 6-chloro-3,4-dihydro-2H-benzo[b][1,4]oxazine-2-carboxylic acid and substituting Example 100B for Example 23B gave the title compound. ¹H NMR (400 MHz, DMSO-d₆) δ ppm 7.72 (s, 1H), 7.66-7.57 (m, 2H), 7.48 (t, J=8.9 Hz, 1H), 7.26 (s, 1H), 7.15 (d, J=8.6 Hz, 1H), 7.05 (dd, J=11.4, 2.9 Hz, 1H), 6.82 (ddd, J=9.0, 3.0, 1.2 Hz, 1H), 5.04 (dd, J=8.2, 5.0 Hz, 1H), 4.46 (s, 2H), 4.03 (dd, J=9.6, 3.1 Hz, 1H), 3.01-2.84 (m, 2H), 2.22 (ddt, J=12.4, 9.4, 2.7 Hz, 1H), 2.11-1.99 (m, 1H), 1.96-1.68 (m, 9H).; MS (ESI⁺) m/z 552 (M+H)⁺.

### Example 100D: 6-chloro-N-{(3S)-4-[2-(4-chloro-3-fluorophenoxy)acetamido]-3-hydroxybicyclo[2.2.2]octan-1-yl}-4-hydroxy-3,4-dihydro-2H-1-benzopyran-2-carboxamide

The methodologies described in Example 89 removing 2-((trimethylsilyl)oxy)ethan-amine, substituting Example 100C for Example 87, and purifying by preparative HPLC [Waters XBridge™ C18 5 μm OBD column, 30×100 mm, flow rate 40 mL/minute, 5-100% gradient of acetonitrile in buffer (0.025 M aqueous ammonium bicarbonate, adjusted to pH 10 with ammonium hydroxide)] gave the title compound. ¹H NMR (400 MHz, DMSO-d₆, dr cis:trans=5:1) δ ppm 7.48 (t, J=8.9 Hz, 6H), 7.42 (s, 1H), 7.37 (dd, J=2.7, 1.0 Hz, 5H), 7.35-7.32 (m, 5H), 7.30 (d, J=2.6 Hz, 1H), 7.26 (s, 6H), 7.22 (dd, J=8.8, 2.7 Hz, 1H), 7.18 (ddd, J=8.7, 2.7, 0.7 Hz, 5H), 7.06 (dd, J=11.4, 2.9 Hz, 6H), 6.91 (d, J=8.7 Hz, 1H), 6.86 (d, J=8.7 Hz, 5H), 6.83 (ddd, J=9.0, 2.9, 1.2 Hz, 7H), 5.66 (d, J=6.4 Hz, 5H), 5.58 (d, J=4.6 Hz, 1H), 5.08 (dd, J=4.9, 2.1 Hz, 6H), 4.77 (dt, J=11.7, 6.1 Hz, 5H), 4.55 (dd, J=11.8, 2.2 Hz, 7H), 4.47 (s, 11H), 4.09-4.02 (m, 6H), 2.34-2.22 (m, 12H), 2.12-2.00 (m, 3H), 2.07 (s, 4H), 2.04-1.89 (m, 11H), 1.86 (dd, J=10.6, 2.7 Hz, 4H), 1.81 (d, J=8.4 Hz, 29H), 1.80-1.66 (m, 5H); MS (ESI⁺) m/z 535 (M−H₂O+H)⁺.

### Example 101: N-{(2S)-4-[2-(4-chloro-3-fluorophenoxy)acetamido]-2-hydroxybicyclo[2.2.2]octan-1-yl}-6-fluoro-3,4-dihydro-2H-1-benzopyran-2-carboxamide (Compound 200)

To a mixture of the product from Example 27A (91 mg, 0.20 mmol) and 6-fluorochroman-2-carboxylic acid (39.2 mg, 0.20 mmol) in N,N-dimethylformamide (1 mL) was added 1-[bis(dimethylamino)methylene]-1H-1,2,3-triazolo[4,5-b]pyridinium 3-oxide hexafluorophosphate (76 mg, 0.20 mmol) and N-ethyl-N-isopropylpropan-2-amine (0.14 mL, 0.80 mmol), and the resulting mixture was stirred at room temperature for 0.5 hour. Water was added, and the mixture was purified by C18 HPLC using a solvent gradient of 5-95% acetonitrile in water (with 0.1% trifluoroacetic acid) to give the title compound (90 mg, 0.17 mmol, 86%). ¹H NMR (400 MHz, DMSO-d₆) δ ppm 8.70 (d, J=7.5 Hz, 2H), 7.49 (t, J=8.9 Hz, 1H), 7.07 (dd, J=11.4, 2.8 Hz, 1H), 6.90-6.82 (m, 1H), 6.82-6.71 (m, 2H), 6.54 (dd, J=8.5, 2.4 Hz, 1H), 4.69 (t, J=5.5 Hz, 1H), 4.47 (s, 2H), 4.45 (dd, J=7.9, 2.9 Hz, 2H), 3.61-3.47 (m, 4H), 2.26 (s, 6H), 2.24 (d, J=2.6 Hz, 1H); MS (ESI⁺) m/z 524 (M+H)⁺.

### Example 102: (2S)—N-{(2S)-4-[2-(4-chloro-3-fluorophenoxy)acetamido]-2-hydroxybicyclo[2.2.2]octan-1-yl}-6-fluoro-3,4-dihydro-2H-1-benzopyran-2-carboxamide (Compound 201)

The product from Example 101 (60 mg, 0.115 mmol) was purified by chiral SFC [Whelk-O®1 (S,S) column] using 40% methanol in CO₂ as the eluent. The title compound was the first of 2 stereoisomers to elute (30 mg). ¹H NMR (400 MHz, DMSO-d₆) δ ppm 7.50 (s, 1H), 7.48 (t, J=8 Hz, 1H), 7.10 (s, 1H), 7.02 (dd, J=10, 3 Hz, 1H), 6.93 (m, 2H), 6.83 (m, 2H), 5.16 (d, J=3 Hz, 1H), 4.45 (m, 1H), 4.43 (s, 2H), 3.97 (m, 1H), 2.79 (m, 1H), 2.67 (m, 1H), 2.25 (m, 2H), 2.11 (m, 1H), 1.65-1.96 (m, 9H); MS (ESI⁺) m/z 521 (M+H)⁺.

### Example 103: (2R)—N-{(2S)-4-[2-(4-chloro-3-fluorophenoxy)acetamido]-2-hydroxybicyclo[2.2.2]octan-1-yl}-6-fluoro-3,4-dihydro-2H-1-benzopyran-2-carboxamide (Compound 202)

The product from Example 101 (60 mg, 0.115 mmol) was purified by chiral SFC [Whelk-O®1 (S,S) column] using 40% methanol in CO₂ as the eluent. The title compound was the second of 2 stereoisomers to elute (29 mg). ¹H NMR (400 MHz, DMSO-d₆) δ ppm 7.50 (s, 1H), 7.48 (t, J=8 Hz, 1H), 7.11 (s, 1H), 7.02 (dd, J=10, 3 Hz, 1H), 6.93 (m, 2H), 6.82 (m, 2H), 5.19 (d, J=3 Hz, 1H), 4.45 (m, 1H), 4.43 (s, 2H), 3.89 (m, 1H), 2.79 (m, 1H), 2.67 (m, 1H), 2.25 (m, 2H), 2.11 (m, 1H), 1.65-1.96 (m, 9H); MS (ESI⁺) m/z 521 (M+H)⁺.

### Example 104: 6-chloro-N-{(2S)-4-[2-(4-chloro-3-fluorophenoxy)acetamido]-2-hydroxybicyclo[2.2.2]octan-1-yl}-3,4-dihydro-2H-1-benzopyran-2-carboxamide (Compound 203)

The title compound was prepared using the procedures described for Example 101, substituting 6-chlorochroman-2-carboxylic acid for 6-fluorochroman-2-carboxylic acid. ¹H NMR (400 MHz, DMSO-d₆) δ ppm 7.52 (s, 1H), 7.48 (t, J=8 Hz, 1H), 7.14 (m, 3H), 7.02 (dd, J=10, 3 Hz, 1H), 6.85 (d, J=8 Hz, 1H), 6.81 (br d, J=8 Hz, 1H), 4.47 (m, 1H), 4.43 (s, 2H), 3.89-4.00 (m, 1H), 2.76 (m, 1H), 2.65 (m, 1H), 2.25 (m, 2H), 2.10 (m, 1H), 1.65-1.96 (m, 9H); MS (ESI⁺) m/z 537 (M+H)⁺.

### Example 105: 6-chloro-N-{3-[2-(4-chloro-3-fluorophenoxy)acetamido]bicyclo[1.1.1]pentan-1-yl}-3,4-dihydro-2H-1-benzopyran-2-carboxamide (Compound 204)

To a mixture of the product from Example 23B (0.150 g, 0.376 mmol) and 6-chlorochroman-2-carboxylic acid (0.100 g, 0.470 mmol) in N,N-dimethylformamide (3.0 mL) was added 1-[bis(dimethylamino)methylene]-1H-1,2,3-triazolo[4,5-b]pyridinium 3-oxid hexafluorophosphate (0.150 g, 0.395 mmol) and N-ethyl-N-isopropylpropan-2-amine (0.26 mL, 1.51 mmol), and the resulting mixture was stirred at room temperature for 16 hours. This mixture was partitioned between saturated, aqueous sodium bicarbonate solution and dichloromethane, and the organic layer was dried over sodium sulfate, filtered, and concentrated under vacuum. The residue was and purified on C18 HPLC using a solvent gradient of 5-100% gradient of acetonitrile in buffer (0.025 M aqueous ammonium bicarbonate, adjusted to pH 10 with ammonium hydroxide) to give the title compound (60 mg, 0.125 mmol, 33% yield). ¹H NMR (501 MHz, DMSO-d₆) δ ppm 8.69 (s, 1H), 8.61 (s, 1H), 7.48 (t, J=8.9 Hz, 1H), 7.12 (s, 2H), 7.06 (dd, J=11.4, 2.8 Hz, 1H), 6.88-6.82 (m, 2H), 4.50-4.43 (m, 3H), 2.83-2.59 (m, 2H), 2.25 (s, 6H), 2.16-2.06 (m, 1H), 1.88-1.77 (m, 1H); MS (ESI⁺) m/z 479 (M+H)⁺.

### Example 106: (2S,4S)-7-chloro-N-{3-[2-(4-chloro-3-fluorophenoxy)acetamido]bicyclo[1.1.1]pentan-1-yl}-6-fluoro-4-hydroxy-3,4-dihydro-2H-1-benzopyran-2-carboxamide (Compound 205)

Example 147 was purified by chiral SFC (supercritical fluid chromatography) using a Whelk-O (S,S) column eluting with 100% CH₃OH in CO₂ with a flow rate of 70 g/minute and back pressure of 100 bar to give the title compound (second enantiomer eluted out of the column, 0.046 g, 0.090 mmol, 53% yield). The stereochemistry of this title compound was arbitrarily assigned (This compound is the enantiomer of Example 107.). ¹H NMR (400 MHz, DMSO-d₆) δ ppm 8.72 (s, 1H), 8.67 (s, 1H), 7.50 (t, J=8.8 Hz, 1H), 7.31 (dd, J=9.8, 1.0 Hz, 1H), 7.12-7.04 (m, 2H), 6.86 (ddd, J=9.0, 2.9, 1.2 Hz, 1H), 5.76 (d, J=6.2 Hz, 1H), 4.79 (dt, J=11.5, 6.0 Hz, 1H), 4.63 (dd, J=11.9, 2.4 Hz, 1H), 4.48 (s, 2H), 2.36 (ddd, J=13.0, 5.8, 2.4 Hz, 1H), 2.28 (s, 6H), 1.74-1.61 (m, 1H); MS (APCI⁺) m/z 495 (M−H₂O+H)⁺.

### Example 107: (2R,4R)-7-chloro-N-{3-[2-(4-chloro-3-fluorophenoxy)acetamido]bicyclo[1.1.1]pentan-1-yl}-6-fluoro-4-hydroxy-3,4-dihydro-2H-1-benzopyran-2-carboxamide (Compound 206)

Example 147 was purified by chiral SFC (supercritical fluid chromatography) using a Whelk-O (S,S) column eluting with 100% CH₃OH in CO₂ with a flow rate of 70 g/minute and back pressure of 100 bar to give the title compound (first enantiomer eluted out of the column, 0.048 g, 0.094 mmol, 55% yield). The stereochemistry of this title compound was arbitrarily assigned (This compound is the enantiomer of Example 106.). ¹H NMR (500 MHz, DMSO-d₆) δ ppm 1H NMR (500 MHz, DMSO-d₆) δ 8.74 (s, 1H), 8.69 (s, 1H), 7.50 (t, J=8.9 Hz, 1H), 7.31 (dd, J=9.9, 0.9 Hz, 1H), 7.11-7.05 (m, 2H), 6.86 (ddd, J=9.0, 3.0, 1.2 Hz, 1H), 5.78 (d, J=6.1 Hz, 1H), 4.79 (dt, J=11.4, 6.0 Hz, 1H), 4.63 (dd, J=11.9, 2.4 Hz, 1H), 4.48 (s, 2H), 2.45-2.31 (m, 1H), 2.28 (s, 6H), 1.73-1.62 (m, 1H); MS (APCI⁺) m/z 495 (M−H₂O+H)⁺.

### Example 108: (2R,4R)—N-{3-[2-(3,4-dichlorophenoxy)acetamido]bicyclo[1.1.1]pentan-1-yl}-6,7-difluoro-4-hydroxy-3,4-dihydro-2H-1-benzopyran-2-carboxamide (Compound 207)

### Example 108A: N-(3-aminobicyclo[1.1.1]pentan-1-yl)-2-(3,4-dichlorophenoxy)acetamide

The reaction and purification conditions described in Example 23A through Example 23B substituting 2-(3,4-dichlorophenoxy)acetic acid for 2-(4-chloro-3-fluorophenoxy)acetic acid gave the title compound. MS (APCI⁺) m/z 301 (M+H)⁺.

### Example 108B: (E)-4-(4,5-difluoro-2-hydroxyphenyl)-4-oxobut-2-enoic Acid

Maleic anhydride (1.90 g, 19.4 mmol) and aluminum chloride (5.17 g, 38.7 mmol) were added to dichloroethane (20 mL) and stirred at 50° C. for 2 minutes. 3,4-Difluoroanisole (2.0 mL, 16.9 mmol) was added dropwise over a period of 2 minutes. The resulting reaction mixture was stirred at 50° C. for 5 hours and then at ambient temperature for 18 hours and was poured into a mixture of concentrated HCl (11.6 M, 20 mL) and ice (about 100 g). When the ice was all melted and while the mixture was still cold, the precipitate was collected by filtration through paper and dried in the vacuum oven to give the title compound (1.54 g, 6.75 mmol, 40% yield). MS (APCI⁺) m/z 301 (M+H)⁺.

### Example 108C: 6,7-difluoro-4-oxochroman-2-carboxylic Acid

The product of Example 108B (340 mg, 1.49 mmol) was suspended in water (7.45 mL) and stirred at ambient temperature. Aqueous NaOH (1.0 M, 1.64 mL) was added dropwise over a period of 2 minutes. The resulting reaction mixture was heated to 100° C. and stirred for 2 minutes and then allowed to cool to ambient temperature over a period of 15 minutes. Aqueous HCl (6 M) was added dropwise to adjust pH to ˜1. The resulting milky solution was partitioned between dichloromethane (2×30 mL) and water (10 mL), dried over sodium sulfate and concentrated under reduced pressure. The residue was purified by preparative HPLC [YMC TriArt™ C18 Hybrid 5 μm column, 50×100 mm, flow rate 140 mL/minute, 0-100% gradient of acetonitrile in buffer (0.1% trifluoroacetic acid)] to give the title compound (0.2 g, 0.88 mmol, 59% yield). MS (APCI⁻) m/z 227 (M−H)⁻.

### Example 108D: (R)-6,7-difluoro-4-oxochroman-2-carboxylic Acid

The product of Example 108C was purified by preparative chiral HPLC [Daicel CHIRALPAK® AD-H 5 μm column, 20×250 mm, flow rate 6 mL/minute, 80% ethanol and 0.1% trifluoroacetic acid in heptane (isocratic gradient)] to give the title compound as the earlier eluting fraction. MS (ESI⁻) m/z 227 (M−H)⁻.

### Example 108E: (2R)—N-{3-[2-(3,4-dichlorophenoxy)acetamido]bicyclo[1.1.1]pentan-1-yl}-6,7-difluoro-4-oxo-3,4-dihydro-2H-1-benzopyran-2-carboxamide (Compound 207E)

1-[Bis(dimethylamino)methylene]-1H-1,2,3-triazolo[4,5-b]pyridinium 3-oxid hexafluorophosphate (HATU, 20 mg, 0.053 mmol) was added to a solution of the product of Example 108D (10 mg, 0.044 mmol), the product of Example 108A (13.2 mg, 0.044 mmol) and triethylamine (0.012 mL, 0.088 mmol) in N,N-dimethylformamide (2.0 mL). The resulting reaction mixture was stirred at ambient temperature for 1 hour. Water (0.3 mL) was added. The resulting solution was filtered through a glass microfiber frit and purified by preparative HPLC [Waters XBridge™ C18 5 μm column, 30×100 mm, flow rate 40 mL/minute, 5-100% gradient of acetonitrile in buffer (0.025 M aqueous ammonium bicarbonate, adjusted to pH 10 with ammonium hydroxide)] to give the title compound (19 mg, 0.038 mmol, 85% yield). ¹H NMR (500 MHz, DMSO-d₆) δ ppm 8.95 (s, 1H), 8.73 (s, 1H), 7.69 (dd, J=10.3, 9.1 Hz, 1H), 7.55 (d, J=8.9 Hz, 1H), 7.31-7.23 (m, 2H), 6.98 (dd, J=9.0, 2.9 Hz, 1H), 5.11 (dd, J=7.5, 6.6 Hz, 1H), 4.49 (s, 2H), 2.97-2.92 (m, 2H), 2.26 (s, 6H); MS (APCI⁺) m/z 511 (M+H)⁺.

### Example 108F: (2R,4R)—N-{3-[2-(3,4-dichlorophenoxy)acetamido]bicyclo[1.1.1]pentan-1-yl}-6,7-difluoro-4-hydroxy-3,4-dihydro-2H-1-benzopyran-2-carboxamide

The product of Example 108E (19 mg, 0.037 mmol) was combined with CH₃OH (1.0 mL) and stirred at ambient temperature. Sodium borohydride (5.6 mg, 0.15 mmol) was added. After stirring at ambient temperature for 30 minutes, saturated NH₄Cl solution was added (0.2 mL), and the reaction mixture was stirred for 10 minutes and then partitioned between dichloromethane (2×5 mL) and saturated sodium bicarbonate (5 mL). The organic phases were combined, dried over sodium sulfate, and concentrated under reduced pressure. The residue was purified by preparative HPLC [YMC TriArt™ C18 Hybrid 5 μm column, 50×100 mm, flow rate 140 mL/minute, 5-100% gradient of acetonitrile in buffer (0.025 M aqueous ammonium bicarbonate, adjusted to pH 10 with ammonium hydroxide)] to give the title compound (19 mg, 0.037 mmol, 100% yield). ¹H NMR (400 MHz, DMSO-d₆) δ ppm 8.72 (s, 1H), 8.66 (s, 1H), 7.55 (d, J=8.9 Hz, 1H), 7.33 (ddd, J=11.4, 9.3, 1.0 Hz, 1H), 7.27 (d, J=2.9 Hz, 1H), 6.99 (dd, J=9.0, 2.9 Hz, 1H), 6.92 (dd, J=11.9, 7.0 Hz, 1H), 5.70 (d, J=6.0 Hz, 1H), 4.77 (dt, J=11.4, 5.8 Hz, 1H), 4.62 (dd, J=11.9, 2.3 Hz, 1H), 4.49 (s, 2H), 2.34 (ddd, J=13.0, 5.9, 2.4 Hz, 1H), 2.28 (s, 6H), 1.75-1.61 (m, 1H); MS (APCI⁺) m/z 495 (M−H₂O+H)⁺.

### Example 109: (2S,4S)—N-{3-[2-(3,4-dichlorophenoxy)acetamido]bicyclo[1.1.1]pentan-1-yl}-6,7-difluoro-4-hydroxy-3,4-dihydro-2H-1-benzopyran-2-carboxamide (Compound 208)

### Example 109A: (S)-6,7-difluoro-4-oxochroman-2-carboxylic Acid

The product of Example 108C was purified by preparative chiral HPLC [Regis® Technologies, Inc. CHIRALPAK® AD-H 5 μm column, 20×250 mm, flow rate 6 mL/minute, 80% ethanol and 0.1% trifluoroacetic acid in heptane (isocratic gradient)] to give the title compound as the later eluting fraction. MS (ESI⁻) m/z 227 (M−H)⁻; Specific rotation [□]_{D}=+42.6° (c 0.27, CH₃OH, 20° C.).

### Example 109B: (2S,4S)—N-{3-[2-(3,4-dichlorophenoxy)acetamido]bicyclo[1.1.1]pentan-1-yl}-6,7-difluoro-4-hydroxy-3,4-dihydro-2H-1-benzopyran-2-carboxamide

The reaction and purification conditions described in Example 108E through Example 108F substituting the product of Example 109A for the product of Example 108D gave the title compound. ¹H NMR (400 MHz, DMSO-d₆) δ ppm 8.72 (s, 1H), 8.67 (s, 1H), 7.55 (d, J=8.9 Hz, 1H), 7.33 (ddd, J=11.4, 9.2, 1.0 Hz, 1H), 7.27 (d, J=2.9 Hz, 1H), 6.99 (dd, J=9.0, 2.9 Hz, 1H), 6.92 (dd, J=11.8, 7.0 Hz, 1H), 5.72 (d, J=5.4 Hz, 1H), 4.81-4.73 (m, 1H), 4.62 (dd, J=11.9, 2.3 Hz, 1H), 4.49 (s, 2H), 2.35 (ddd, J=13.0, 5.8, 2.4 Hz, 1H), 2.28 (s, 6H), 1.69 (ddd, J=12.9, 12.0, 10.6 Hz, 1H); MS (APCI⁺) m/z 495 (M−H₂O+H)⁺.

### Example 110: 6-chloro-N-{3-[2-(4-chloro-3-fluorophenoxy)acetamido]bicyclo[1.1.1]pentan-1-yl}-4-{[(1s,3s)-3-hydroxycyclobutyl]amino}-3,4-dihydro-2H-1-benzopyran-2-carboxamide (Compound 209)

The methodologies described in Example 89 substituting (1s,3s)-3-aminocyclobutan-1-ol hydrochloride for 2-((trimethylsilyl)oxy)ethanamine and purifying using preparative HPLC [Waters XBridge™ C18 5 μm OBD column, 30×100 mm, flow rate 40 mL/minute, 5-100% gradient of acetonitrile in buffer (0.025 M aqueous ammonium bicarbonate, adjusted to pH 10 with ammonium hydroxide)] gave the title compound as the major product (55% yield), along with the product of Example 91 and Example 241. ¹H NMR (501 MHz, DMSO-d₆, dr 1:1) δ ppm 8.72 (s, 1H), 8.72 (s, 1H), 8.68 (s, 1H), 8.63 (s, 1H), 7.53-7.46 (m, 3H), 7.26-7.13 (m, 3H), 7.08 (dd, J=11.4, 2.9 Hz, 2H), 6.93-6.83 (m, 4H), 4.90 (dd, J=5.9, 4.7 Hz, 2H), 4.63 (dd, J=11.1, 2.6 Hz, 1H), 4.51 (dd, J=11.1, 2.5 Hz, 1H), 4.48 (s, 4H), 3.86 (s, 1H), 3.75 (dh, J=20.9, 7.2 Hz, 2H), 3.62 (s, 1H), 2.76 (d, J=19.3 Hz, 2H), 2.51-2.38 (m, 2H), 2.32 (ddd, J=13.1, 5.4, 2.5 Hz, 1H), 2.28 (s, 6H), 2.28 (s, 6H), 2.22 (s, 1H), 2.17 (s, 1H), 2.10 (dt, J=13.7, 2.9 Hz, 1H), 1.76-1.53 (m, 6H); MS (APCI⁺) m/z 564 (M+H)⁺.

### Example 111: 1-{[6-chloro-2-({3-[2-(4-chloro-3-fluorophenoxy)acetamido]bicyclo[1.1.1]pentan-1-yl}carbamoyl)-3,4-dihydro-2H-1-benzopyran-4-yl]amino}cyclopropane-1-carboxylic acid (Compound 210)

The methodologies described in Example 89 substituting 1-aminocyclopropanecarboxylic acid for 2-((trimethylsilyl)oxy)ethanamine gave the title compound as the major product (51% yield), along with the product of Example 91. ¹H NMR (501 MHz, DMSO-d₆, dr 1:1) δ ppm 8.91 (s, 1H), 8.83 (s, 1H), 8.76 (s, 1H), 8.75 (s, 1H), 7.72 (d, J=2.5 Hz, 1H), 7.53-7.46 (m, 3H), 7.39 (ddd, J=18.0, 8.8, 2.6 Hz, 2H), 7.09-7.01 (m, 5H), 6.86 (ddd, J=9.0, 2.9, 1.1 Hz, 2H), 5.17 (dd, J=10.7, 6.3 Hz, 1H), 4.87 (t, J=3.8 Hz, 1H), 4.74 (dd, J=11.8, 2.6 Hz, 1H), 4.66 (dd, J=11.1, 2.2 Hz, 1H), 4.49 (d, J=1.2 Hz, 4H), 2.65-2.52 (m, 2H), 2.31 (s, 6H), 2.30 (s, 6H), 2.18-2.08 (m, 1H), 2.07 (s, 1H), 2.03 (dt, J=13.0, 11.0 Hz, 1H), 1.67-1.49 (m, 3H), 1.48-1.40 (m, 2H), 1.33 (dddd, J=10.1, 7.1, 4.8, 2.1 Hz, 2H); MS (APCI⁺) m/z 578 (M+H)⁺.

### Example 112: methyl [(2S)-6-chloro-2-({3-[2-(4-chloro-3-fluorophenoxy)acetamido]bicyclo[1.1.1]pentan-1-yl}carbamoyl)-2,3-dihydro-4H-1,4-benzoxazin-4-yl]acetate (Compound 211)

To a solution of the product of Example 4 (0.100 g, 0.208 mmol) in N,N-dimethylformamide (1.5 mL) was added potassium carbonate (0.086 g, 0.63 mmol), sodium iodide (0.016 g, 0.10 mmol) and methyl bromoacetate (0.02 mL, 0.2 mmol). This reaction mixture was allowed to stir at 75° C. overnight, and despite incomplete conversion, was concentrated, and purified by preparative HPLC (Waters XBridge™ C18 5 μm OBD column, 30×100 mm, flow rate 40 mL/minute, 5-100% gradient of acetonitrile in 0.1% trifluoroacetic acid/water) to give the title compound (0,015 g, 0.027 mmol, 13% yield). ¹H NMR (501 MHz, DMSO-d₆) δ ppm 8.73 (d, J=9.6 Hz, 2H), 7.49 (t, J=8.9 Hz, 1H), 7.07 (dd, J=11.4, 2.9 Hz, 1H), 6.88-6.81 (m, 2H), 6.66 (d, J=2.4 Hz, 1H), 6.62 (dd, J=8.4, 2.4 Hz, 1H), 6.53 (s, 1H), 4.54 (dd, J=8.0, 2.9 Hz, 1H), 4.48 (s, 2H), 4.33 (d, J=18.4 Hz, 1H), 4.20 (d, J=18.3 Hz, 1H), 3.65 (s, 3H), 3.54 (dd, J=12.1, 2.9 Hz, 1H), 3.35-3.28 (m, 1H), 2.27 (s, 6H); MS (APCI⁺) m/z 553 (M+H)⁺.

### Example 113: 6-chloro-N-{3-[2-(4-chloro-3-fluorophenoxy)acetamido]bicyclo[1.1.1]pentan-1-yl}-4-[(3,3-difluorocyclobutyl)amino]-3,4-dihydro-2H-1-benzopyran-2-carboxamide (Compound 212)

The methodologies described in Example 89 substituting 3,3-difluorocyclobutanamine hydrochloride for 2-((trimethylsilyl)oxy)ethanamine gave the title compound as the major product (72% yield), along with the product of Example 91 and Example 241. ¹H NMR (500 MHz, DMSO-d₆, dr 1.5:1) δ ppm 8.97 (s, 1H), 8.90 (s, 1H), 8.78 (d, J=4.0 Hz, 2H), 7.75 (d, J=2.5 Hz, 1H), 7.61 (d, J=2.6 Hz, 1H), 7.49 (t, J=8.8 Hz, 2H), 7.44 (dd, J=8.8, 2.6 Hz, 1H), 7.39 (dd, J=8.8, 2.5 Hz, 1H), 7.11-7.02 (m, 4H), 6.86 (ddd, J=9.0, 2.8, 1.2 Hz, 2H), 4.81 (dd, J=10.7, 6.4 Hz, 1H), 4.72 (dd, J=12.2, 2.6 Hz, 1H), 4.58 (dd, J=11.4, 2.2 Hz, 1H), 4.49 (d, J=1.8 Hz, 4H), 4.02 (qd, J=8.2, 5.5 Hz, 2H), 3.19-2.94 (m, 2H), 2.63 (ddd, J=13.1, 6.3, 2.3 Hz, 1H), 2.56 (dt, J=15.3, 2.8 Hz, 1H), 2.31 (s, 6H), 2.30 (s, 4H), 2.17-2.08 (m, 1H), 2.07 (s, 1H), 1.96 (dt, J=13.0, 11.1 Hz, 1H); MS (APCI⁺) m/z 585 (M+H)⁺.

### Example 114: N-{3-[(6-chloro-4-hydroxy-3,4-dihydro-2H-1-benzopyran-2-carbonyl)amino]bicyclo[1.1.1]pentan-1-yl}-5-(difluoromethyl)pyrazine-2-carboxamide (Compound 213)

### Example 114A: tert-butyl (3-(6-chloro-4-oxochroman-2-carboxamido)bicyclo[1.1.1]pentan-1-yl)carbamate

6-Chloro-4-oxochroman-2-carboxylic acid (Princeton, 1.314 g, 5.80 mmol), tert-butyl (3-aminobicyclo[1.1.1]pentan-1-yl)carbamate (Combi-Blocks, 1.15 g, 5.80 mmol) and triethylamine (1.62 mL) were combined with N,N-dimethylformamide (20 mL) and stirred at ambient temperature. 1-[Bis(dimethylamino)methylene]-1H-1,2,3-triazolo[4,5-b]pyridinium 3-oxid hexafluorophosphate (HATU, 2.43 g, 6.38 mmol) was added portionwise over 1 minute. The resulting mixture suspension was stirred at ambient temperature for 1 hour. Water (100 mL) was added to the resulting thick mixture and stirred for 10 minutes. The precipitate was collected on filter paper, and the filter cake was washed with more water (2×10 mL) and dried in the vacuum oven to give the title compound (2.3 g, 5.65 mmol, 97% yield). MS (APCI⁻) (m/z 405 (M−H)⁻.

### Example 114B: tert-butyl (3-((2R,4R)-6-chloro-4-hydroxychroman-2-carboxamido)bicyclo[1.1.1]pentan-1-yl)carbamate

The reaction and purification conditions described in Example 108F substituting the product of Example 114A for the product of Example 108E gave the title compound. ¹H NMR (501 MHz, DMSO-d₆) δ ppm 8.62 (s, 1H), 7.50 (s, 1H), 7.37 (dd, J=2.7, 1.0 Hz, 1H), 7.19 (ddd, J=8.7, 2.7, 0.7 Hz, 1H), 6.87 (d, J=8.7 Hz, 1H), 5.68 (d, J=5.9 Hz, 1H), 4.79 (dt, J=11.3, 5.6 Hz, 1H), 4.57 (dd, J=12.0, 2.2 Hz, 1H), 2.33 (ddd, J=12.9, 5.9, 2.3 Hz, 1H), 2.15 (s, 6H), 1.74-1.62 (m, 1H), 1.37 (br s, 9H); MS (APCI⁺) m/z 391 (M−H₂O+H)⁺.

### Example 114C: N-(3-aminobicyclo[1.1.1]pentan-1-yl)-6-chloro-4-hydroxychroman-2-carboxamide, 2 trifluoroacetic Acid

The product of Example 114B (200 mg, 0.489 mmol) was stirred in dichloromethane (3 mL) at ambient temperature. Trifluoroacetic acid (3 mL) was added in one portion. After stirring for 6 hours, the reaction mixture was concentrated under reduced pressure to give the title compound (0.27 g, 0.50 mmol, 103% yield). MS (APCI⁺) (m/z 309 (M+H)⁺.

### Example 114D: N-{3-[(6-chloro-4-hydroxy-3,4-dihydro-2H-1-benzopyran-2-carbonyl)amino]bicyclo[1.1.1]pentan-1-yl}-5-(difluoromethyl)pyrazine-2-carboxamide

The reaction and purification conditions described in Example 108E substituting the product of Example 114C for the product of Example 108A, and 5-(difluoromethyl)pyrazine-2-carboxylic acid (PharmaBlock) for the product of Example 108D gave the title compound. ¹H NMR (400 MHz, DMSO-d₆) (˜60:40 mixture of cis and trans) δ ppm 9.58 (s, 1H), 9.22 (d, J=1.4 Hz, 1H), 8.96 (d, J=1.3 Hz, 1H), 8.78-8.64 (m, 1H), 7.40-7.02 (m, 3H), 6.94-6.81 (m, 1H), 5.64 (br s, 1H), 4.82-4.73 (m, 1H), 4.63-4.50 (m, 1H), 2.39-1.83 (m, 7H), 1.73-1.61 (m, 1H); MS (APCI⁺) m/z 391 (M−H₂O+H)⁺.

### Example 115: N-{(3S)-4-[(6-chloro-4-oxo-3,4-dihydro-2H-1-benzopyran-2-carbonyl)amino]-3-hydroxybicyclo[2.2.2]octan-1-yl}-3-(difluoromethyl)-1,2-oxazole-5-carboxamide (Compound 214)

### Example 115A: 2,2-dimethoxyacetaldehyde oxime

To a solution of hydroxylamine, hydrochloric acid (2.0 g, 28.8 mmol) in water (20 mL) was added a solution of NaHCO₃ (3.87 g, 46.1 mmol) in water (20 mL) at 20° C., then a solution of 2,2-dimethoxyacetaldehyde (5 g, 28.8 mmol) in 2-methoxy-2-methylpropane (30 mL) was added at 20° C., and the resulting solution was stirred for 12 hours at 20° C. The mixture was extracted with ethyl acetate (2×100 mL), and the combined organic fractions was dried over anhydrous Na₂SO₄, filtered and concentrated under reduced pressure to give the title compound (3.5 g, 26.4, 92% yield). ¹H NMR (400 MHz, CDCl₃) δ ppm 8.71 (s, 1H), 7.37 (d, J=5.26 Hz, 1H), 4.86 (d, J=5.26 Hz, 1H), 3.37-3.44 (m, 6H).

### Example 115B: N-hydroxy-2,2-dimethoxyacetimidoyl chloride

To a solution of the product of Example 115A (3.5 g, 26.4 mmol) in N,N-dimethylformamide, (50 mL) was added N-chlorosuccinimide (NCS, 4.24 g, 31.7 mmol) at 0° C. The reaction mixture was then allowed to warm to 20° C. with stirring over 16 hours. The reaction mixture was diluted with water (150 mL) and extracted with CH₂Cl₂ (3×200 mL). The combined organic fractions were washed with brine (3×200 mL), filtered and concentrated under reduced pressure to give the title compound (3.3 g, 19.3 mmol, 73% yield). ¹H NMR (400 MHz, CDCl₃) δ ppm 8.58 (s, 1H), 4.91 (s, 1H), 3.42 (s, 6H).

### Example 115C: methyl 3-(dimethoxymethyl)isoxazole-5-carboxylate

To a solution of methyl propiolate (3.15 g, 37.5 mmol) in toluene (100 mL) at 5° C. was added the product of Example 115B (3.2 g, 18.75 mmol). Then N,N-diisopropylethylamine (3.60 mL, 20.6 mmol) was added dropwise at 5° C., and the mixture was allowed to warm to ambient temperature and was stirred for 12 hours. The reaction mixture was diluted with water (100 mL) and extracted with ethyl acetate (2×100 mL). The combined organic fractions were dried over anhydrous Na₂SO₄, filtered, and concentrated under reduced pressure. The residue was purified by column chromatography (SiO₂, petroleum ether and ethyl acetate (100:1 to 50:1)) to give the title compound (2.2 g, 10.4 mmol, 55% yield). ¹H NMR (400 MHz, CDCl₃) δ ppm 7.01 (s, 1H), 5.53 (s, 1H), 3.98 (s, 3H), 3.43 (s, 6H).

### Example 115D: methyl 3-formylisoxazole-5-carboxylate

A mixture of the product of Example 115C (2.1 g, 9.92 mmol) in trifluoroacetic acid (30 mL) and water (3 mL) was stirred for 12 hours at 20° C. The mixture was diluted with water (100 mL) and was extracted with CH₂Cl₂ (3×100 mL). The combined organic fractions were washed with saturated, aqueous NaHCO₃ (carefully), washed with brine (100 mL), and dried over anhydrous Na₂SO₄. The mixture was filtered, and the filtrate was concentrated under reduced pressure to give the title compound. ¹H NMR (400 MHz, CDCl₃) δ ppm 9.98-10.23 (m, 1H), 5.23 (s, 1H), 3.94 (s, 3H), 3.35 (s, 2H).

### Example 115E: methyl 3-(difluoromethyl)isoxazole-5-carboxylate

To a solution of the product of Example 115D (1.05 g, 6.43 mmol) in CH₂Cl₂ (50 mL) at −40° C. under N2 was added diethylaminosulfur trifluoride (DAST, 1.7 mL, 12.9 mmol), and the resulting solution was allowed to warm to 20° C. and was stirred for 12 hours. The reaction was quenched with saturated, aqueous NaHCO₃, and the layers were separated. The organic fraction was washed with brine (100 mL), dried over anhydrous Na₂SO₄ and concentrated under reduced pressure to give the title compound (1.0 g, 5.1 mmol, 79% yield). ¹H NMR (400 MHz, CDCl₃) δ ppm 7.15 (s, 1H), 6.66-6.99 (m, 1H), 3.98-4.03 (m, 3H).

### Example 115F: 3-(difluoromethyl)isoxazole-5-carboxylic Acid

To a solution of the product of Example 115E (0.95 g, 4.8 mmol) in tetrahydrofuran (20 mL), methanol (5 mL) and water (5 mL) was added LiOH (0.23 g, 9.7 mmol) at 0° C., and the resulting solution was stirred for 2 hours at 20° C. The material was concentrated under reduced pressure, and the residue was diluted with water (20 mL) and extracted with CH₂Cl₂ (50 mL). The aqueous layer was adjusted to pH=1 by addition of aqueous HCl (0.5 M), and the resulting mixture was extracted with ethyl acetate (2×50 mL). The ethyl acetate extracts were combined, dried over anhydrous Na₂SO₄ and concentrated under reduced pressure to give the title compound (0.73 g, 4.4 mmol, 91% yield). ¹H NMR (400 MHz, CDCl₃) δ ppm 7.50 (s, 1H), 7.18-7.47 (m, 1H).

### Example 115G: N-[(3S)-4-amino-3-hydroxybicyclo[2.2.2]octan-1-yl]-3-(difluoromethyl)-1,2-oxazole-5-carboxamide hydrochloride

The reaction and purification conditions described in Example 1B [substituting the product of Example 115F for 2-(4-chloro-3-fluorophenoxy)acetic acid, 1-[bis(dimethylamino)methylene]-1H-1,2,3-triazolo[4,5-b]pyridinium 3-oxid hexafluorophosphate (HATU) for (1-cyano-2-ethoxy-2-oxoethylidenaminooxy)dimethylamino-morpholino-carbenium hexafluorophosphate (COMU), and the product of Example 85C for the product of Example 1A], followed by the subsequent reaction and purification conditions described in Example 1C (substituting 4 N HCl in dioxane for trifluoroacetic acid) gave the title compound. MS (APCI⁺) m/z 302 (M+H)⁺.

### Example 115H: N-{(3S)-4-[(6-chloro-4-oxo-3,4-dihydro-2H-1-benzopyran-2-carbonyl)amino]-3-hydroxybicyclo[2.2.2]octan-1-yl}-3-(difluoromethyl)-1,2-oxazole-5-carboxamide

The reaction and purification conditions described in Example 114A substituting the product of Example 115G for tert-butyl (3-aminobicyclo[1.1.1]pentan-1-yl)carbamate gave the title compound. ¹H NMR (400 MHz, DMSO-d₆) δ ppm 8.30-8.28 (m, 1H), 7.63-7.56 (m, 2H), 7.48-7.26 (m, 3H), 7.17-7.09 (m, 1H), 5.11-5.02 (m, 2H), 4.11-3.96 (m, 1H), 2.98-2.86 (m, 2H), 2.32 (ddd, J=12.5, 9.4, 2.3 Hz, 1H), 2.07-1.69 (m, 9H); MS (ESI⁺) (m/z 510 (M+H)⁺.

### Example 116: N-[(3S)-4-{[(2R,4R)-6-chloro-4-hydroxy-3,4-dihydro-2H-1-benzopyran-2-carbonyl]amino}-3-hydroxybicyclo[2.2.2]octan-1-yl]-3-(difluoromethyl)-1,2-oxazole-5-carboxamide (Compound 215)

The reaction and purification conditions described in Example 108F substituting the product of Example 115H for the product of Example 108E gave the title compound as an earlier eluting diastereomer during HPLC purification. Stereochemistry has been arbitrarily assigned. ¹H NMR (400 MHz, DMSO-d₆) δ ppm 8.34 (s, 1H), 7.47-7.32 (m, 3H), 7.24-7.17 (m, 2H), 6.86 (d, J=8.7 Hz, 1H), 5.70 (d, J=5.2 Hz, 1H), 5.20 (d, J=4.3 Hz, 1H), 4.84-4.75 (m, 1H), 4.61 (dd, J=11.4, 2.4 Hz, 1H), 4.11-4.05 (m, 1H), 2.42-2.32 (m, 2H), 2.27-2.18 (m, 1H), 2.09-1.83 (m, 8H), 1.73 (ddd, J=13.1, 11.5, 10.4 Hz, 1H); MS (ESI⁻) m/z 510 (M−H)⁻.

### Example 117: N-[(3S)-4-{[(2S,4S)-6-chloro-4-hydroxy-3,4-dihydro-2H-1-benzopyran-2-carbonyl]amino}-3-hydroxybicyclo[2.2.2]octan-1-yl]-3-(difluoromethyl)-1,2-oxazole-5-carboxamide (Compound 216)

The reaction and purification conditions described in Example 108F substituting the product of Example 115H for the product of Example 108E gave the title compound as the later eluting diastereomer during HPLC purification. Stereochemistry has been arbitrarily assigned. ¹H NMR (501 MHz, DMSO-d₆) δ ppm 8.34 (s, 1H), 7.41 (s, 1H), 7.38 (dd, J=2.7, 1.0 Hz, 1H), 7.33 (t, J=53.0 Hz, 1H), 7.20 (ddd, J=8.7, 2.7, 0.7 Hz, 1H), 7.16 (s, 1H), 5.71 (s, 1H), 5.22 (s, 1H), 4.80 (dd, J=10.4, 5.8 Hz, 1H), 4.61 (dd, J=11.5, 2.4 Hz, 1H), 4.04 (dd, J=9.7, 3.3 Hz, 1H), 2.41-2.32 (m, 2H), 2.28-2.21 (m, 1H), 2.09-2.01 (m, 1H), 1.99-1.80 (m, 8H), 1.80-1.71 (m, 1H); MS (ESI⁻) m/z 510 (M−H)⁻.

### Example 118: N-{3-[(6-chloro-4-oxo-3,4-dihydro-2H-1-benzopyran-2-carbonyl)amino]bicyclo[1.1.1]pentan-1-yl}-5-(difluoromethyl)pyrazine-2-carboxamide (Compound 217)

The title compound was prepared using the methodologies described above. ¹H NMR (400 MHz, DMSO-d₆) δ ppm 9.62 (s, 1H), 9.24 (d, J=1.4 Hz, 1H), 9.00-8.97 (m, 2H), 7.67-7.63 (m, 2H), 7.20 (t, J=54.0 Hz, 1H), 7.20-7.16 (m, 1H), 5.11 (t, J=7.1 Hz, 1H), 2.97 (d, J=7.0 Hz, 2H), 2.36 (s, 6H); MS (APCI⁺) m/z 463 (M+H)⁺.

### Example 119: N-(3-{[rac-(2R,4R)-6-chloro-4-hydroxy-3,4-dihydro-2H-1-benzopyran-2-carbonyl]amino}bicyclo[1.1.1]pentan-1-yl)-5-(difluoromethyl)pyrazine-2-carboxamide (Compound 218)

The reaction and purification conditions described in Example 108F substituting the product of Example 118 for the product of Example 108E gave the title compound. ¹H NMR (501 MHz, DMSO-d₆) δ ppm 9.62 (s, 1H), 9.25 (d, J=1.4 Hz, 1H), 9.00 (d, J=1.4 Hz, 1H), 8.72 (s, 1H), 7.38 (dd, J=2.7, 1.0 Hz, 1H), 7.21 (t, J=54.0 Hz, 1H), 7.21 (ddd, J=8.7, 2.7, 0.7 Hz, 1H), 6.89 (d, J=8.7 Hz, 1H), 5.71 (s, 1H), 4.81 (dd, J=10.7, 6.0 Hz, 1H), 4.61 (dd, J=12.0, 2.3 Hz, 1H), 2.39 (s, 6H), 2.38-2.34 (m, 1H), 1.71 (ddd, J=12.9, 12.1, 10.8 Hz, 1H); MS (ESI⁻) m/z 463 (M−H)⁻.

### Example 120: 6-chloro-N-{3-[2-(4-chloro-3-fluorophenoxy)acetamido]bicyclo[1.1.1]pentan-1-yl}-4-[(methanesulfonyl)amino]-3,4-dihydro-2H-1-benzopyran-2-carboxamide (Compound 219)

To a solution of the product of Example 206 (0.043 g, 0.088 mmol) in dichloromethane (0.5 mL) was added triethylamine (0.015 mL, 0.11 mmol) and methanesulfonyl chloride (8.9 μL, 0.092 mmol). This reaction mixture was allowed to stir at ambient temperature for 2.5 hours, was diluted with water (1 mL), extracted with dichloromethane (3 mL×3), and concentrated. The residue was purified by preparative HPLC (Waters XBridge™ C18 5 μm OBD column, 30×100 mm, flow rate 40 mL/minute, 5-100% gradient of acetonitrile in 0.1% trifluoroacetic acid/water) to afford the title compound (20 mg, 0.035 mmol, 40% yield) as a mixture of diastereomers (dr 1:1). ¹H NMR (400 MHz, DMSO-d₆, dr 1:1) δ ppm 8.77 (s, 1H), 8.75 (s, 1H), 8.73 (s, 1H), 8.72 (s, 1H), 7.79 (d, J=7.7 Hz, 1H), 7.67 (d, J=8.9 Hz, 1H), 7.50 (td, J=8.9, 1.0 Hz, 2H), 7.42-7.32 (m, 2H), 7.26 (ddd, J=16.1, 8.8, 2.7 Hz, 2H), 7.08 (dd, J=11.3, 2.9 Hz, 2H), 6.93 (dd, J=15.4, 8.8 Hz, 2H), 6.86 (ddd, J=9.0, 2.8, 1.2 Hz, 2H), 4.74 (ddd, J=11.3, 8.8, 5.8 Hz, 1H), 4.69-4.50 (m, 3H), 4.48 (d, J=1.4 Hz, 4H), 3.10 (s, 6H), 2.51-2.43 (m, 1H), 2.29 (s, 6H), 2.28 (s, 6H), 2.27-2.21 (m, 1H), 2.09-1.97 (m, 1H), 1.82 (dt, J=13.2, 11.7 Hz, 1H); MS (APCI⁺) m/z 572 (M+H)⁺.

### Example 121: 4-acetamido-6-chloro-N-{3-[2-(4-chloro-3-fluorophenoxy)acetamido]bicyclo[1.1.1]pentan-1-yl}-3,4-dihydro-2H-1-benzopyran-2-carboxamide (Compound 220)

To a solution of the product of Example 206 (0.038 g, 0.076 mmol) in dichloromethane (0.43 mL) was added triethylamine (0.013 mL, 0.092 mmol) and acetyl chloride (5.7 μL, 0.080 mmol). This reaction mixture was allowed to stir at ambient temperature for 3.5 hours, was diluted with water (1 mL), extracted with dichloromethane (3 mL×3), and concentrated. The residue was purified by preparative HPLC (Waters XBridge™ C18 5 μm OBD column, 30×100 mm, flow rate 40 mL/minute, 5-100% gradient of acetonitrile in 0.1% trifluoroacetic acid/water) to afford the title compound (10 mg, 0.019 mmol, 24% yield) as a mixture of diastereomers (dr 1:1). ¹H NMR (501 MHz, DMSO-d₆, dr 1:1) δ ppm 8.74 (s, 1H), 8.74-8.71 (m, 3H), 8.41 (d, J=7.8 Hz, 1H), 8.26 (d, J=8.5 Hz, 1H), 7.49 (td, J=8.9, 1.3 Hz, 2H), 7.29-7.20 (m, 2H), 7.23-7.18 (m, 1H), 7.13-7.04 (m, 3H), 6.94 (dd, J=16.6, 8.8 Hz, 2H), 6.86 (ddt, J=9.0, 2.8, 1.3 Hz, 2H), 5.15 (ddd, J=11.2, 8.8, 6.0 Hz, 1H), 4.91 (dt, J=8.5, 4.6 Hz, 1H), 4.67 (dd, J=11.7, 2.2 Hz, 1H), 4.54 (dd, J=9.9, 3.0 Hz, 1H), 4.48 (d, J=2.1 Hz, 4H), 2.29 (s, 6H), 2.28 (s, 6H), 2.08 (ddd, J=14.0, 4.6, 3.1 Hz, 1H), 1.97 (ddd, J=14.2, 9.9, 4.9 Hz, 1H), 1.93 (s, 3H), 1.87 (s, 3H), 1.75 (dt, J=13.2, 11.6 Hz, 1H); MS (APCI⁺) m/z 536 (M+H)⁺.

### Example 122: N-{3-[2-(4-chloro-3-fluorophenoxy)acetamido]bicyclo[1.1.1]pentan-1-yl}-6,7-dimethoxy-4-oxo-4H-1-benzopyran-2-carboxamide (Compound 221)

To a mixture of the product of Example 23B (0.085 g, 0.30 mmol) and 6,7-dimethoxy-4-oxo-4H-chromene-2-carboxylic acid (0.078 g, 0.31 mmol, Valencia, M E, et al. Eur. J. Med. Chem., 2018, 156, 534-553.) in N,N-dimethylformamide (1.7 mL) was added triethylamine (0.17 mL, 1.2 mmol) followed by 1-[bis(dimethylamino)methylene]-1H-1,2,3-triazolo[4,5-b]pyridinium 3-oxid hexafluorophosphate (HATU, 0.13 g, 0.33 mmol). This reaction mixture was allowed to stir at ambient temperature for 16 hours. Then the reaction mixture was diluted with water (0.5 mL), filtered, and purified by preparative HPLC (Phenomenex® Luna® C18(2) 10 μm 100 Å AXIA™ column (250 mm×50 mm). A 30-100% gradient of acetonitrile (A) and 0.1% trifluoroacetic acid in water (B) is used over 25 minutes, at a flow rate of 50 mL/minute) to give the title compound (0.065 g, 0.13 mmol, 42% yield). ¹H NMR (501 MHz, DMSO-d₆) δ ppm 9.61 (s, 1H), 8.79 (s, 1H), 7.50 (t, J=8.9 Hz, 1H), 7.35 (s, 1H), 7.22 (s, 1H), 7.09 (dd, J=11.4, 2.9 Hz, 1H), 6.87 (ddd, J=9.0, 2.9, 1.2 Hz, 1H), 6.74 (s, 1H), 4.51 (s, 2H), 3.94 (s, 3H), 3.87 (s, 3H), 2.39 (s, 6H); MS (APCI⁺) m/z 517 (M+H)⁺.

### Example 123: N-{3-[2-(4-chloro-3-fluorophenoxy)acetamido]bicyclo[1.1.1]pentan-1-yl}-7-fluoro-4-hydroxy-3,4-dihydro-2H-1-benzopyran-2-carboxamide (Compound 222)

To a suspension of the product of Example 214 (0.115 g, 0.242 mmol) in methanol (4.3 mL) was added sodium borohydride (0.046 g, 1.211 mmol). This reaction mixture was allowed to stir at ambient temperature for 30 minutes, then was diluted with water (1 mL) and sodium bicarbonate (saturated aqueous solution, 1 mL), and extracted with ethyl acetate (3×5 mL). The combined organic layers were dried (Na₂SO₄) and concentrated under heated N2. The residue was purified by preparative HPLC (Phenomenex® Luna® C18(2) 10 μm 100 Å AXIA™ column (250 mm×50 mm). A 30-100% gradient of acetonitrile (A) and 0.1% trifluoroacetic acid in water (B) is used over 25 minutes, at a flow rate of 50 mL/minute) to give the title compound (0.025 g, 0.052 mmol, 22% yield). ¹H NMR (400 MHz, DMSO-d₆) δ ppm 8.72 (s, 1H), 8.66 (s, 1H), 7.50 (t, J=8.9 Hz, 1H), 7.45-7.36 (m, 1H), 7.08 (dd, J=11.4, 2.8 Hz, 1H), 6.86 (ddd, J=9.0, 2.9, 1.2 Hz, 1H), 6.76 (td, J=8.6, 2.6 Hz, 1H), 6.68 (dd, J=10.5, 2.6 Hz, 1H), 5.57 (d, J=6.3 Hz, 1H), 4.77 (dt, J=11.3, 6.0 Hz, 1H), 4.61 (dd, J=11.9, 2.4 Hz, 1H), 4.48 (s, 2H), 2.38-2.30 (m, 1H), 2.28 (s, 6H), 1.77-1.64 (m, 1H); MS (APCI⁺) m/z 461 (M−H₂O+H)⁺.

### Example 124: (2R)-6-chloro-N-{3-[2-(4-chloro-3-fluorophenoxy)acetamido]bicyclo[1.1.1]pentan-1-yl}-4-oxo-3,4-dihydro-2H-1-benzopyran-2-carboxamide (Compound 223)

### Example 124A: (R)-6-chloro-4-oxochroman-2-carboxylic Acid

6-Chloro-4-oxo-3,4-dihydro-2H-1-benzopyran-2-carboxylic acid (Princeton) was purified by preparative chiral SFC [performed on a Thar 200 preparative SFC (SFC-5) system using a Daicel CHTRALPAK® AD-H, 30×250 mm I.D., 5 μm column. The column was heated at 38° C., and the backpressure regulator was set to maintain 100 bar. The mobile phase was 40% methanol in carbon dioxide at a flowrate of 80 g/minute (isocratic gradient)] to give the title compound as the earlier eluting fraction. MS (ESI⁻) m/z 225 (M−H)⁻.

### Example 124B: (2R)-6-chloro-N-{3-[2-(4-chloro-3-fluorophenoxy)acetamido]bicyclo[1.1.1]pentan-1-yl}-4-oxo-3,4-dihydro-2H-1-benzopyran-2-carboxamide

The reaction and purification conditions described in Example 108E substituting the product of Example 23B for the product of Example 108A, and the product of Example 124A for the product of Example 108D gave the title compound. ¹H NMR (400 MHz, DMSO-d₆) δ ppm 8.94 (s, 1H), 8.72 (s, 1H), 7.68-7.60 (m, 2H), 7.49 (t, J=8.9 Hz, 1H), 7.21-7.13 (m, 1H), 7.07 (dd, J=11.4, 2.9 Hz, 1H), 6.85 (ddd, J=8.8, 2.8, 1.2 Hz, 1H), 5.09 (t, J=7.1 Hz, 1H), 4.47 (s, 2H), 2.95 (d, J=7.1 Hz, 2H), 2.26 (s, 6H); MS (ESI⁻) m/z 491 (M−H)⁻.

### Example 125: (2S)-6-chloro-N-{3-[2-(4-chloro-3-fluorophenoxy)acetamido]bicyclo[1.1.1]pentan-1-yl}-4-oxo-3,4-dihydro-2H-1-benzopyran-2-carboxamide (Compound 224)

### Example 125A: (S)-6-chloro-4-oxochroman-2-carboxylic Acid

6-Chloro-4-oxo-3,4-dihydro-2H-1-benzopyran-2-carboxylic acid (Princeton) was purified by preparative chiral SFC [performed on a Thar 200 preparative SFC (SFC-5) system using a Chiralpak® AD-H, 30×250 mm I.D., 5 μm column. The column was heated at 38° C., and the backpressure regulator was set to maintain 100 bar. The mobile phase was 40% methanol in carbon dioxide at a flowrate of 80 g/minute (isocratic gradient)] to give the title compound as the later eluting fraction. MS (ESI⁻) m/z 225 (M−H)⁻; Specific rotation [□]_{D}=+58° (c 0.62, CH₃OH, 20° C.).

### Example 125B: (2S)-6-chloro-N-{3-[2-(4-chloro-3-fluorophenoxy)acetamido]bicyclo[1.1.1]pentan-1-yl}-4-oxo-3,4-dihydro-2H-1-benzopyran-2-carboxamide

The reaction and purification conditions described in Example 108E substituting the product of Example 23B for the product of Example 108A, and the product of Example 125A for the product of Example 108D gave the title compound. ¹H NMR (400 MHz, DMSO-d₆) δ ppm 8.94 (s, 1H), 8.72 (s, 1H), 7.68-7.60 (m, 2H), 7.49 (t, J=8.9 Hz, 1H), 7.21-7.13 (m, 1H), 7.07 (dd, J=11.4, 2.8 Hz, 1H), 6.85 (ddd, J=8.9, 2.9, 1.2 Hz, 1H), 5.09 (t, J=7.1 Hz, 1H), 4.47 (s, 2H), 2.95 (d, J=7.1 Hz, 2H), 2.26 (s, 6H); MS (ESI⁻) m/z 491 (M−H)⁻.

### Example 126: (2R)-6-chloro-4-oxo-N-[3-(2-{[6-(trifluoromethyl)pyridin-3-yl]oxy}acetamido)bicyclo[1.1.1]pentan-1-yl]-3,4-dihydro-2H-1-benzopyran-2-carboxamide (Compound 225)

### Example 126A: N-(3-aminobicyclo[1.1.1]pentan-1-yl)-2-((6-(trifluoromethyl)pyridin-3-yl)oxy)acetamide

The reaction and purification conditions described in Examples 23A through 23B substituting 2-((6-(trifluoromethyl)pyridin-3-yl)oxy)acetic acid (prepared as described in International Patent Publication WO2017/193030 A1) for 2-(4-chloro-3-fluorophenoxy)acetic acid gave the title compound. MS (APCI⁺) m/z 302 (M+H)⁺.

### Example 126B: (2R)-6-chloro-4-oxo-N-[3-(2-{[6-(trifluoromethyl)pyridin-3-yl]oxy}acetamido)bicyclo[1.1.1]pentan-1-yl]-3,4-dihydro-2H-1-benzopyran-2-carboxamide

The reaction and purification conditions described in Example 108E substituting the product of Example 126A for the product of Example 108A, and the product of Example 124A for the product of Example 108D gave the title compound. ¹H NMR (400 MHz, DMSO-d₆) δ ppm 8.94 (s, 1H), 8.81 (s, 1H), 8.46 (d, J=2.9 Hz, 1H), 7.86 (d, J=8.7 Hz, 1H), 7.66-7.61 (m, 2H), 7.59-7.54 (m, 1H), 7.19-7.14 (m, 1H), 5.09 (d, J=14.2 Hz, 1H), 4.66 (s, 2H), 2.95 (d, J=7.0 Hz, 2H), 2.26 (s, 6H); MS (APCI⁺) m/z 510 (M+H)⁺.

### Example 127: (2R,4R)-6-chloro-4-hydroxy-N-[3-(2-{[6-(trifluoromethyl)pyridin-3-yl]oxy}acetamido)bicyclo[1.1.1]pentan-1-yl]-3,4-dihydro-2H-1-benzopyran-2-carboxamide (Compound 226)

The reaction and purification conditions described in Example 108F substituting the product of Example 126 for the product of Example 108E gave the title compound. ¹H NMR (400 MHz, DMSO-d₆) δ ppm 8.81 (s, 1H), 8.68 (s, 1H), 8.47 (d, J=2.8 Hz, 1H), 7.87 (d, J=8.7 Hz, 1H), 7.61-7.54 (m, 1H), 7.38 (dd, J=2.7, 1.0 Hz, 1H), 7.20 (ddd, J=8.7, 2.7, 0.7 Hz, 1H), 6.88 (d, J=8.7 Hz, 1H), 5.69 (d, J=5.7 Hz, 1H), 4.85-4.75 (m, 1H), 4.67 (s, 2H), 4.60 (dd, J=12.0, 2.3 Hz, 1H), 2.35 (ddd, J=12.9, 5.9, 2.3 Hz, 1H), 2.29 (s, 6H), 1.76-1.63 (m, 1H); MS (APCI⁺) m/z 494 (M−H₂O+H)⁺.

### Example 128: (2R,4R)-6-chloro-4-hydroxy-N-(3-{2-[(2-methoxypyrimidin-5-yl)oxy]acetamido}bicyclo[1.1.1]pentan-1-yl)-3,4-dihydro-2H-1-benzopyran-2-carboxamide (Compound 227)

### Example 128A: (R)-tert-butyl (3-(6-chloro-4-oxochroman-2-carboxamido)bicyclo[1.1.1]pentan-1-yl)carbamate

The reaction and purification conditions described in Example 114A substituting the product of Example 124A for 6-chloro-4-oxochroman-2-carboxylic acid gave the title compound. MS (ESI⁺) m/z 424 (M+NH₄).

### Example 128B: (R)—N-(3-aminobicyclo[1.1.1]pentan-1-yl)-6-chloro-4-oxochroman-2-carboxamide, 4 trifluoroacetic Acid

Trifluoroacetic acid (1.0 mL) was added to a solution of the product of Example 128A (0.32 g, 0.787 mmol) in dichloromethane (2 mL) stirring at 0° C. The reaction mixture was slowly warmed up to ambient temperature over 10 minutes and then with continued stirring for 2 hours. The resulting solution was concentrated under reduced pressure to give the title compound (0.6 g, 0.79 mmol, 100% yield). MS (ESI⁺) m/z 307 (M+H)⁺.

### Example 128C: N,N′-[(2S)-2-hydroxybicyclo[2.2.2]octane-1,4-diyl]bis{2-[(2-methoxypyrimidin-5-yl)oxy]acetamide}

To a solution of 2-methoxypyrimidin-5-ol (6.1 g, 48.4 mmol) in N,N-dimethylformamide (50 mL) at ambient temperature was added potassium carbonate (13.37 g, 97 mmol) and tert-butyl bromoacetate (8.16 mL, 55.6 mmol). This suspension was then stirred at 65° C. for 1.5 hours. The reaction mixture was cooled to ambient temperature and was diluted with ethyl acetate (50 mL) and water (50 mL). The aqueous layer was extracted with ethyl acetate (3×100 mL). The combined organic layer was dried over anhydrous Na₂SO₄ and filtered. The filtrate was concentrated under reduced pressure to give tert-butyl 2-((2-methoxypyrimidin-5-yl)oxy)acetate as a crude oil, which was used without further purification. This crude was dissolved in methanol (90 mL) and water (30.0 mL), to this mixture was added aqueous NaOH (5.0 M, 48.0 mL). This reaction mixture was stirred at ambient temperature for 12 hours. Solvent was removed under reduced pressure to give a brown solid. The crude was then dissolved in water (200 mL) and pH was adjusted to ˜1 with aqueous 1 N HCl. The resulting precipitate was isolated by filtration, washed with water and air-dried to give the title compound (5.9 g, 32.0 mmol, 66.7% yield). ¹H NMR (501 MHz, DMSO-d₆) δ ppm 8.37 (s, 2H), 4.79 (s, 2H), 3.87 (s, 3H).

### Example 128D: (2R,4R)-6-chloro-4-hydroxy-N-(3-{2-[(2-methoxypyrimidin-5-yl)oxy]acetamido}bicyclo[1.1.1]pentan-1-yl)-3,4-dihydro-2H-1-benzopyran-2-carboxamide

The reaction and purification conditions described in Example 108F substituting the product of Example 234 for the product of Example 108E gave the title compound. ¹H NMR (500 MHz, DMSO-d₆) δ ppm 8.75 (s, 1H), 8.69 (s, 1H), 8.36 (s, 2H), 7.40-7.36 (m, 1H), 7.20 (dd, J=8.7, 2.7 Hz, 1H), 6.88 (d, J=8.7 Hz, 1H), 5.71 (s, 1H), 4.80 (dd, J=10.9, 5.9 Hz, 1H), 4.63-4.57 (m, 1H), 4.55 (s, 2H), 3.86 (s, 3H), 2.35 (dd, J=12.8, 5.1 Hz, 1H), 2.28 (s, 6H), 1.69 (q, J=11.9 Hz, 1H); MS (APCI⁺) m/z 475 (M+H)⁺.

### Example 129: (2R,4R)-6-chloro-4-hydroxy-N-[3-(2-{[2-(trifluoromethyl)pyrimidin-5-yl]oxy}acetamido)bicyclo[1.1.1]pentan-1-yl]-3,4-dihydro-2H-1-benzopyran-2-carboxamide (Compound 228)

The reaction and purification conditions described in Example 108F substituting the product of Example 230 for the product of Example 108E gave the title compound. ¹H NMR (500 MHz, DMSO-d₆) δ ppm 8.86 (s, 1H), 8.76 (s, 2H), 8.70 (s, 1H), 7.38 (dd, J=2.7, 1.0 Hz, 1H), 7.20 (ddd, J=8.7, 2.7, 0.7 Hz, 1H), 6.88 (d, J=8.7 Hz, 1H), 5.70 (d, J=4.8 Hz, 1H), 4.82-4.78 (m, 3H), 4.60 (dd, J=12.0, 2.3 Hz, 1H), 2.39-2.31 (m, 1H), 2.29 (s, 6H), 1.74-1.64 (m, 1H); MS (APCI⁺) m/z 495 (M−H₂O+H)⁺.

### Example 130: (2R,4R)-6-chloro-N-{4-[2-(4-chloro-3-fluorophenoxy)acetamido]bicyclo[2.2.2]octan-1-yl}-4-hydroxy-3,4-dihydro-2H-1-benzopyran-2-carboxamide (Compound 229)

Example 238 was purified by chiral SFC (supercritical fluid chromatography) using a Daicel CHIRALPAK® AD-H column eluting with 100% CH₃OH in CO₂ with a flow rate of 80 g/minute and back pressure of 120 bar to give the title compound (first enantiomer eluted out of the column, 0.0174 g, 0.032 mmol, 41% yield). The stereochemistry of this title compound was arbitrarily assigned (This compound is the enantiomer of Example 207.). ¹H NMR (400 MHz, DMSO-d₆) δ ppm 7.52-7.43 (m, 2H), 7.37 (d, J=2.6 Hz, 1H), 7.30 (s, 1H), 7.17 (dd, J=8.7, 2.7 Hz, 1H), 7.02 (dd, J=11.4, 2.8 Hz, 1H), 6.86 (d, J=8.7 Hz, 1H), 6.81 (ddd, J=8.9, 2.9, 1.2 Hz, 1H), 5.67 (s, 1H), 4.77 (dd, J=10.7, 5.9 Hz, 1H), 4.55 (dd, J=11.8, 2.2 Hz, 1H), 4.44 (s, 2H), 2.27 (ddd, J=12.9, 5.9, 2.3 Hz, 1H), 1.92 (s, 12H), 1.72 (dt, J=12.7, 11.0 Hz, 1H); MS (APCI⁺) m/z 520 (M−H₂O+H)⁺.

### Example 131: N-{3-[2-(4-chloro-3-fluorophenoxy)acetamido]bicyclo[1.1.1]pentan-1-yl}-4-oxo-7-(trifluoromethyl)-4H-1-benzopyran-2-carboxamide (Compound 230)

### Example 131A: ethyl 4-oxo-7-(trifluoromethyl)-4H-chromene-2-carboxylate

In a 20 mL vial was added 1-(2-hydroxy-4-(trifluoromethyl)phenyl)ethanone (408 mg, 2 mmol) in ethanol (8 mL) followed by diethyl oxalate (0.595 mL, 4.40 mmol). To this solution was added sodium ethoxide (21% weight in ethanol, 2.99 mL, 8.00 mmol). The reaction was heated to 80° C. for 1 hour, at which point the reaction had solidified. The reaction mixture was then cooled to ambient temperature, sulfuric acid (0.533 mL, 10.0 mmol) was added, and the mixture was heated to 80° C. for one hour. Then the mixture was cooled to ambient temperature and volatiles were removed under vacuum. To the residue was added ethyl acetate and water. The aqueous layer was removed and the organic phase was washed with brine. The organic fraction was dried over Na₂SO₄ and concentrated. The resulting title compound was carried directly onto the hydrolysis without purification. MS (ESI⁺) m/z 287 (M+H)⁺.

### Example 131B: 4-oxo-7-(trifluoromethyl)-4H-chromene-2-carboxylic Acid

Example 131A was dissolved in acetic acid (4 mL). Concentrated HCl (1.2 mL) was added, and the reaction mixture was stirred overnight at 80° C. The reaction mixture was then cooled to ambient temperature and concentrated in vacuo. The residue was diluted with water (4 mL), vortexed, and centrifuged. The water was decanted, the process was repeated one more time, and the title compound was dried under vacuum. ¹H NMR (500 MHz, DMSO-d₆) δ ppm 8.23 (dd, J=8.5, 1.0 Hz, 1H), 8.14 (dd, J=1.6, 0.9 Hz, 1H), 7.84 (dd, J=8.4, 1.7 Hz, 1H), 6.98 (s, 1H); MS (ESI⁻) m/z 257 (M−H)⁻.

### Example 131C: N-{3-[2-(4-chloro-3-fluorophenoxy)acetamido]bicyclo[1.1.1]pentan-1-yl}-4-oxo-7-(trifluoromethyl)-4H-1-benzopyran-2-carboxamide

Example 131B (54.4 mg, 0.21 mmol, 1.2 eq) was weighed into a 4 mL vial. To this was added N,N-dimethylacetamide (0.5 mL), followed by 1-[bis(dimethylamino)methylene]-1H-1,2,3-triazolo[4,5-b]pyridinium 3-oxid hexafluorophosphate (HATU, 67 mg, 0.18 mmol, 1.0 eq) in N,N-dimethylacetamide (0.4 mL), and then the product of Example 23B (50 mg, 0.18 mmol, 1.0 eq) in N,N-dimethylacetamide (0.4 mL). To this reaction mixture was added N,N-diisopropylethylamine (92 uL, 0.53 mmol, 3.0 equivalents). The reaction mixture was stirred overnight at ambient temperature, filtered, and purified by preparative HPLC (Phenomenex® Luna® C18(2) 10 μm 100 Å AXIA™ column (250 mm×50 mm). A 30-100% gradient of acetonitrile (A) and 0.1% trifluoroacetic acid in water (B) was used over 25 minutes, at a flow rate of 50 mL/minute) to give the title compound (38.1 mg, 41% yield). ¹H NMR (500 MHz, DMSO-d₆) δ ppm 9.87 (s, 1H), 8.91 (s, 1H), 8.27 (dt, J=7.8, 0.8 Hz, 1H), 8.15 (dt, J=1.6, 0.7 Hz, 1H), 7.91-7.85 (m, 1H), 7.50 (t, J=8.8 Hz, 1H), 7.08 (dd, J=11.3, 2.8 Hz, 1H), 6.91 (s, 1H), 6.88 (ddd, J=9.0, 2.9, 1.1 Hz, 1H), 4.50 (s, 2H), 2.41 (s, 6H); MS (APCI⁺) m/z 525 (M+H)⁺.

### Example 132: 6-chloro-N-{3-[2-(4-chloro-3-fluorophenoxy)acetamido]bicyclo[1.1.1]pentan-1-yl}-7-fluoro-4-oxo-4H-1-benzopyran-2-carboxamide (Compound 231)

### Example 132A: 6-chloro-7-fluoro-4-oxo-4H-chromene-2-carboxylic Acid

A solution of 1-(5-chloro-4-fluoro-2-hydroxyphenyl)ethanone (0.478 g, 2.54 mmol, International Patent Publication WO2012/135009, 2012) and diethyl oxalate (0.76 mL, 5.6 mmol) in ethanol (1.7 mL) was added dropwise to a solution of sodium ethoxide (2.8 mL, 7.6 mmol, 21 weight % in ethanol). The reaction mixture was heated to 80° C. for 1 hour and then cooled to ambient temperature. Then water (5 mL) and diethyl ether (5 mL) were added and the suspension was stirred as concentrated HCl (2 mL) was added to adjust the pH to about 2. The organic phase was separated, washed twice with brine, dried with Na₂SO₄, filtered, and concentrated. The brown solid/oil was dried a second time from ethyl acetate to give a yellow solid, which was dissolved in sulfuric acid (0.78 mL, 14.6 mmol) and heated to 70° C. for 24 hours. Additional sulfuric acid was added (0.4 mL) and the reaction mixture was heated to 75° C. for 5.5 hours. The reaction mixture was then cooled to ambient temperature, diluted with water, and filtered to give the title intermediate as a brown solid. ¹H NMR (400 MHz, DMSO-d₆) δ ppm 8.17 (d, J=8.1 Hz, 1H), 8.02 (d, J=9.6 Hz, 1H), 6.97 (s, 1H); MS (APCI⁺) m/z 243 (M+H)⁺.

### Example 132B: 6-chloro-N-{3-[2-(4-chloro-3-fluorophenoxy)acetamido]bicyclo[1.1.1]pentan-1-yl}-7-fluoro-4-oxo-4H-1-benzopyran-2-carboxamide

The methodologies described in Example 122 substituting Example 132A for 6,7-dimethoxy-4-oxo-4H-chromene-2-carboxylic acid gave the title compound. ¹H NMR (400 MHz, DMSO-d₆) δ ppm 9.67 (s, 1H), 8.79 (s, 1H), 8.17 (d, J=8.1 Hz, 1H), 7.78 (d, J=9.3 Hz, 1H), 7.50 (t, J=8.9 Hz, 1H), 7.09 (dd, J=11.4, 2.9 Hz, 1H), 6.87 (ddd, J=9.1, 2.9, 1.3 Hz, 1H), 6.84 (s, 1H), 4.50 (s, 2H), 2.39 (s, 6H); MS (APCI⁺) m/z 509 (M+H)⁺.

### Example 133: 7-chloro-N-{3-[2-(4-chloro-3-fluorophenoxy)acetamido]bicyclo[1.1.1]pentan-1-yl}-6-fluoro-4-oxo-4H-1-benzopyran-2-carboxamide (Compound 232)

### Example 133A: ethyl 7-chloro-6-fluoro-4-oxo-4H-chromene-2-carboxylate

The methodologies described in Example 131A substituting 1-(4-chloro-5-fluoro-2-hydroxyphenyl)ethanone for 1-(2-hydroxy-4-(trifluoromethyl)phenyl)ethanone gave the title compound. MS (ESI⁺) m/z 271 (M+H)⁺.

### Example 133B: 7-chloro-6-fluoro-4-oxo-4H-chromene-2-carboxylic acid

The methodologies described in Example 131B substituting Example 133A for Example 131A gave the title compound. MS (ESI⁻) m/z 241 (M−H)⁻.

### Example 133C: 7-chloro-N-{3-[2-(4-chloro-3-fluorophenoxy)acetamido]bicyclo[1.1.1]pentan-1-yl}-6-fluoro-4-oxo-4H-1-benzopyran-2-carboxamide

The methodologies described in Example 131C substituting Example 133B for Example 131B gave the title compound. ¹H NMR (400 MHz, DMSO-d₆) δ ppm 9.83 (s, 1H), 8.89 (s, 1H), 8.06 (d, J=5.9 Hz, 1H), 7.93 (d, J=8.6 Hz, 1H), 7.50 (t, J=8.8 Hz, 1H), 7.07 (dd, J=11.2, 2.8 Hz, 1H), 6.88 (dd, J=9.1, 2.8 Hz, 1H), 6.85 (s, 1H), 4.50 (s, 2H), 2.40 (s, 6H); MS (APCI⁺) m/z 509 (M+H)⁺.

### Example 134: (2R,4R)-6-chloro-N-(3-{2-[(5-chloropyridin-2-yl)oxy]acetamido}bicyclo[1.1.1]pentan-1-yl)-4-hydroxy-3,4-dihydro-2H-1-benzopyran-2-carboxamide (Compound 233)

The reaction and purification conditions described in Example 108F substituting the product of Example 232 for the product of Example 108E gave the title compound. ¹H NMR (500 MHz, DMSO-d₆) δ ppm 8.65 (d, J=5.4 Hz, 2H), 8.18 (d, J=2.9 Hz, 1H), 7.84 (dd, J=8.8, 2.7 Hz, 1H), 7.38 (dd, J=2.6, 0.9 Hz, 1H), 7.19 (dd, J=8.9, 2.5 Hz, 1H), 6.95 (dd, J=8.8, 0.6 Hz, 1H), 6.88 (d, J=8.7 Hz, 1H), 5.68 (s, 1H), 4.80 (dd, J=10.8, 5.9 Hz, 1H), 4.67 (s, 2H), 4.59 (dd, J=12.0, 2.2 Hz, 1H), 2.34 (ddd, J=13.0, 5.9, 2.3 Hz, 1H), 2.25 (s, 6H), 1.75-1.62 (m, 1H); MS (ESI⁺) m/z 476 (M−H)⁻.

### Example 135: rac-(2R,4R)-6-chloro-N-{3-[2-(4-chloro-3-fluorophenoxy)acetamido]bicyclo[1.1.1]pentan-1-yl}-7-fluoro-4-hydroxy-3,4-dihydro-2H-1-benzopyran-2-carboxamide (Compound 234)

The methodologies described in Example 123 substituting Example 132 for Example 214 gave the title compound as solely the cis diastereomer. ¹H NMR (500 MHz, DMSO-d₆) δ ppm 8.73 (s, 1H), 8.70 (s, 1H), 7.53-7.45 (m, 2H), 7.08 (dd, J=11.4, 2.8 Hz, 1H), 6.92 (d, J=10.6 Hz, 1H), 6.86 (ddd, J=9.0, 2.8, 1.2 Hz, 1H), 5.74 (s, 1H), 4.82-4.75 (m, 1H), 4.66 (dd, J=11.8, 2.4 Hz, 1H), 4.49 (s, 2H), 2.35 (ddd, J=13.0, 5.8, 2.5 Hz, 1H), 2.29 (s, 6H), 1.71 (ddd, J=13.0, 11.9, 10.7 Hz, 1H); MS (APCI⁺) m/z 495 (M−H₂O+H)⁺.

### Example 136: 7-chloro-N-{3-[2-(4-chloro-3-fluorophenoxy)acetamido]bicyclo[1.1.1]pentan-1-yl}-4-oxo-4H-1-benzopyran-2-carboxamide (Compound 235)

### Example 136A: ethyl 7-chloro-4-oxo-4H-chromene-2-carboxylate

The methodologies described in Example 131A substituting 1-(4-chloro-2-hydroxyphenyl)ethanone for 1-(2-hydroxy-4-(trifluoromethyl)phenyl)ethanone gave the title compound. ¹H NMR (400 MHz, DMSO-d₆) δ ppm 8.05 (d, J=8.5 Hz, 1H), 7.99 (d, J=1.9 Hz, 1H), 7.60 (dd, J=8.5, 2.0 Hz, 1H), 6.98 (s, 1H), 4.40 (q, J=7.1 Hz, 2H), 1.35 (t, J=7.1 Hz, 3H); MS (ESI⁺) m/z 253 (M+H)⁺.

### Example 136B: 7-chloro-4-oxo-4H-chromene-2-carboxylic Acid

The methodologies described in Example 131B substituting Example 136A for Example 131A gave the title compound. ¹H NMR (400 MHz, DMSO-d₆) δ ppm 8.05 (d, J=8.6 Hz, 1H), 7.96 (d, J=1.9 Hz, 1H), 7.59 (dd, J=8.6, 1.9 Hz, 1H), 6.92 (s, 1H); MS (ESI⁺) m/z 225 (M+H)⁺.

### Example 136C: 7-chloro-N-{3-[2-(4-chloro-3-fluorophenoxy)acetamido]bicyclo[1.1.1]pentan-1-yl}-4-oxo-4H-1-benzopyran-2-carboxamide

The methodologies described in Example 131C substituting Example 136B for Example 131B gave the title compound. ¹H NMR (400 MHz, DMSO-d₆) δ ppm 8.06 (d, J=8.5 Hz, 1H), 7.88 (t, J=1.7 Hz, 1H), 7.60 (dd, J=8.6, 1.8 Hz, 1H), 7.50 (t, J=8.8 Hz, 1H), 7.07 (dd, J=11.2, 2.8 Hz, 1H), 6.88 (dd, J=9.0, 2.8 Hz, 1H), 6.84 (d, J=1.2 Hz, 1H), 4.50 (s, 2H), 2.41 (s, 6H); MS (APCI⁺) m/z 491 (M+H)⁺.

### Example 137: 6-chloro-N-{3-[2-(4-chloro-3-fluorophenoxy)acetamido]bicyclo[1.1.1]pentan-1-yl}-7-methyl-4-oxo-4H-1-benzopyran-2-carboxamide (Compound 236)

### Example 137A: ethyl 6-chloro-7-methyl-4-oxo-4H-chromene-2-carboxylate

The methodologies described in Example 131A substituting 1-(5-chloro-2-hydroxy-4-methylphenyl)ethanone for 1-(2-hydroxy-4-(trifluoromethyl)phenyl)ethanone gave the title compound. ¹H NMR (400 MHz, DMSO-d₆) δ ppm 7.91 (s, 1H), 7.81 (d, J=1.0 Hz, 1H), 6.91 (s, 1H), 4.36 (q, J=7.1 Hz, 2H), 2.44 (d, J=0.8 Hz, 3H), 1.31 (t, J=7.1 Hz, 3H); MS (ESI⁺) m/z 267 (M+H)⁺.

### Example 137B: 6-chloro-7-methyl-4-oxo-4H-chromene-2-carboxylic acid

The methodologies described in Example 131B substituting Example 137A for Example 131A gave the title compound. ¹H NMR (400 MHz, DMSO-d₆) δ ppm 7.95 (s, 1H), 7.82 (d, J=1.0 Hz, 1H), 6.90 (s, 1H), 2.48 (d, J=0.8 Hz, 3H); MS (ESI⁺) m/z 239 (M+H)⁺.

### Example 137C: 6-chloro-N-{3-[2-(4-chloro-3-fluorophenoxy)acetamido]bicyclo[1.1.1]pentan-1-yl}-7-methyl-4-oxo-4H-1-benzopyran-2-carboxamide

The methodologies described in Example 131C substituting Example 137B for Example 131B gave the title compound. ¹H NMR (400 MHz, DMSO-d₆) δ ppm 9.81 (s, 1H), 8.88 (s, 1H), 7.97 (s, 1H), 7.77 (s, 1H), 7.50 (t, J=8.9 Hz, 1H), 7.07 (dd, J=11.3, 2.8 Hz, 1H), 6.88 (dd, J=9.1, 2.8 Hz, 1H), 6.83 (s, 1H), 4.50 (s, 2H), 2.50 (s, 3H), 2.40 (s, 6H).

### Example 138: (2S,4S)-6-chloro-N-{3-[2-(4-chloro-3-fluorophenoxy)acetamido]bicyclo[1.1.1]pentan-1-yl}-4-hydroxy-4-methyl-3,4-dihydro-2H-1-benzopyran-2-carboxamide (Compound 237)

The methodologies described in Example 182 gave the title compound (0.0311 g, 0.112 mmol, 6% yield) (as the first eluting isomer). The stereochemistry of this title compound was arbitrarily assigned (This compound is the enantiomer of Examples 181 and diastereomer of Examples 182 and 189). ¹H NMR (500 MHz, DMSO-d₆) δ ppm 8.73 (s, 2H), 7.50 (t, J=8.9 Hz, 1H), 7.44 (d, J=2.7 Hz, 1H), 7.18 (dd, J=8.8, 2.7 Hz, 1H), 7.08 (dd, J=11.4, 2.9 Hz, 1H), 6.90-6.83 (m, 2H), 5.41 (s, 1H), 4.58 (dd, J=12.6, 2.4 Hz, 1H), 4.48 (s, 2H), 2.29 (s, 6H), 2.11 (dd, J=13.2, 2.5 Hz, 1H), 1.93 (t, J=12.9 Hz, 1H), 1.45 (s, 3H); MS (APCI⁺) m/z 491 (M−H₂O+H)⁺.

### Example 139: (2R,4R)-6-chloro-N-{(2S)-4-[2-(4-chloro-3-fluorophenoxy)acetamido]-2-hydroxybicyclo[2.2.2]octan-1-yl}-4-hydroxy-3,4-dihydro-2H-1-benzopyran-2-carboxamide (Compound 238)

The methodologies described in Example 122 with the following modifications: (1) substituting 6-chloro-4-hydroxychroman-2-carboxylic acid for 6,7-dimethoxy-4-oxo-4H-chromene-2-carboxylic acid, (2) substituting the product of Example 27A for Example 23B, (3) increasing the temperature to 60° C. and reaction time to 6 days, (4) and purifying by preparative HPLC [Waters XBridge™ C18 5 μm OBD column, 30×100 mm, flow rate 40 mL/minute, 5-100% gradient of acetonitrile in buffer (0.025 M aqueous ammonium bicarbonate, adjusted to pH 10 with ammonium hydroxide)] gave the title compound (as the first eluting isomer) and its diastereomer, Example 140. The exact stereochemistry was arbitrarily assigned. ¹H NMR (500 MHz, DMSO-d₆) δ ppm 7.53 (s, 1H), 7.48 (t, J=8.9 Hz, 1H), 7.37 (dd, J=2.6, 0.9 Hz, 1H), 7.21-7.16 (m, 2H), 7.03 (dd, J=11.4, 2.9 Hz, 1H), 6.85 (d, J=8.7 Hz, 1H), 6.83-6.78 (m, 1H), 5.70 (s, 1H), 5.15 (s, 1H), 4.82-4.75 (m, 1H), 4.60 (dd, J=11.5, 2.4 Hz, 1H), 4.44 (s, 2H), 4.03 (d, J=9.5 Hz, 1H), 2.37-2.25 (m, 2H), 2.18 (m, 1H), 1.89 (dd, J=24.1, 7.9 Hz, 1H), 1.81 (m, 6H), 1.79-1.66 (m, 2H); MS (APCI⁺) m/z 535 (M−H₂O+H)⁺.

### Example 140: (2S,4S)-6-chloro-N-{(2S)-4-[2-(4-chloro-3-fluorophenoxy)acetamido]-2-hydroxybicyclo[2.2.2]octan-1-yl}-4-hydroxy-3,4-dihydro-2H-1-benzopyran-2-carboxamide (Compound 239)

The methodologies described in Example 122 with the following modifications: (1) substituting 6-chloro-4-hydroxychroman-2-carboxylic acid for 6,7-dimethoxy-4-oxo-4H-chromene-2-carboxylic acid, (2) substituting the product of Example 27A for Example 23B, (3) increasing the temperature to 60° C. and reaction time to 6 days, (4) and purifying by preparative HPLC [Waters XBridge™ C18 5 μm OBD column, 30×100 mm, flow rate 40 mL/minute, 5-100% gradient of acetonitrile in buffer (0.025 M aqueous ammonium bicarbonate, adjusted to pH 10 with ammonium hydroxide)] gave the title compound (as the second eluting isomer) and its diastereomer, Example 139. The exact stereochemistry was arbitrarily assigned. ¹H NMR (500 MHz, DMSO-d₆) δ ppm 7.53 (s, 1H), 7.48 (t, J=8.9 Hz, 1H), 7.38 (dd, J=2.8, 1.0 Hz, 1H), 7.20 (dd, J=8.6, 2.6 Hz, 1H), 7.13 (s, 1H), 7.03 (dd, J=11.4, 2.9 Hz, 1H), 6.84 (d, J=8.8 Hz, 1H), 6.83-6.77 (m, 1H), 5.71 (s, 1H), 5.17 (d, J=4.4 Hz, 1H), 4.79 (s, 1H), 4.60 (dd, J=11.5, 2.4 Hz, 1H), 4.44 (s, 2H), 3.99 (s, 1H), 2.36-2.26 (m, 1H), 2.22 (d, J=9.8 Hz, 1H), 1.91 (q, J=10.7, 7.7 Hz, 3H), 1.84-1.68 (m, 5H); MS (APCI⁺) m/z 535 (M−H₂O+H)⁺.

### Example 141: 6-chloro-N-{3-[2-(4-chloro-3-fluorophenoxy)acetamido]bicyclo[1.1.1]pentan-1-yl}-2-methyl-3,4-dihydro-2H-1-benzopyran-2-carboxamide (Compound 240)

The methodologies described in Example 122 substituting 6-chloro-2-methylchroman-2-carboxylic acid (U.S. Pat. Appl. Publ. (2017), US 20170305891 A1) for 6,7-dimethoxy-4-oxo-4H-chromene-2-carboxylic acid gave the title compound. ¹H NMR (500 MHz, DMSO-d₆) δ ppm 8.69 (s, 1H), 8.43 (s, 1H), 7.49 (t, J=8.9 Hz, 1H), 7.14 (tt, J=4.6, 2.1 Hz, 2H), 7.07 (dd, J=11.4, 2.8 Hz, 1H), 6.91-6.87 (m, 1H), 6.85 (ddd, J=9.0, 2.9, 1.2 Hz, 1H), 4.46 (s, 2H), 2.70 (dt, J=16.9, 5.9 Hz, 1H), 2.60-2.52 (m, 1H), 2.21 (s, 6H), 2.13 (dt, J=13.4, 5.9 Hz, 1H), 1.76 (ddd, J=13.8, 8.6, 5.5 Hz, 1H), 1.39 (s, 3H); MS (APCI⁺) m/z 493 (M+H)⁺.

### Example 142: (2R,4R)—N-{3-[2-(4-chloro-3-fluorophenoxy)acetamido]bicyclo[1.1.1]pentan-1-yl}-6-fluoro-4-hydroxy-7-methyl-3,4-dihydro-2H-1-benzopyran-2-carboxamide (Compound 241)

Example 248 was purified by chiral SFC (supercritical fluid chromatography) using a Daicel CHIRALPAK® AD-H column eluting with 100% CH₃OH in CO₂ with a flow rate of 42 g/minute and back pressure of 100 bar to give the title compound (first isomer eluted out of the column, 0.035 g, 0.071 mmol, 70% yield). The stereochemistry of this title compound was arbitrarily assigned (This compound is the enantiomer of Examples 143.). ¹H NMR (400 MHz, DMSO-d₆) δ ppm 8.71 (s, 1H), 8.62 (s, 1H), 7.50 (t, J=8.9 Hz, 1H), 7.12-7.03 (m, 2H), 6.86 (dd, J=8.9, 2.3 Hz, 1H), 6.75 (d, J=6.7 Hz, 1H), 5.59 (s, 1H), 4.76 (s, 1H), 4.52 (dd, J=12.2, 2.3 Hz, 1H), 4.48 (s, 2H), 2.32 (m, 1H), 2.28 (s, 6H), 2.16 (s, 3H), 1.66 (q, J=12.1 Hz, 1H); MS (APCI⁺) m/z 475 (M−H₂O+H)⁺.

### Example 143: (2S,4S)—N-{3-[2-(4-chloro-3-fluorophenoxy)acetamido]bicyclo[1.1.1]pentan-1-yl}-6-fluoro-4-hydroxy-7-methyl-3,4-dihydro-2H-1-benzopyran-2-carboxamide (Compound 242)

Example 248 was purified by chiral SFC (supercritical fluid chromatography) using a Daicel CHIRALPAK® AD-H column eluting with 100% CH₃OH in CO₂ with a flow rate of 42 g/minute and back pressure of 100 bar to give the title compound (second isomer eluted out of the column, 0.035 g, 0.071 mmol, 70% yield). The stereochemistry of this title compound was arbitrarily assigned (This compound is the enantiomer of Examples 142.). ¹H NMR (400 MHz, DMSO-d₆) δ ppm 8.71 (s, 1H), 8.62 (s, 1H), 7.50 (t, J=8.9 Hz, 1H), 7.12-7.03 (m, 2H), 6.90-6.82 (m, 1H), 6.75 (d, J=6.6 Hz, 1H), 5.59 (d, J=5.7 Hz, 1H), 4.76 (s, 1H), 4.52 (dd, J=11.9, 2.3 Hz, 1H), 4.48 (s, 2H), 2.37-2.28 (m, 1H), 2.28 (s, 6H), 2.18-2.14 (m, 3H), 1.66 (q, J=12.1 Hz, 1H); MS (APCI⁺) m/z 475 (M−H₂O+H)⁺.

### Example 144: N-{3-[2-(4-chloro-3-fluorophenoxy)acetamido]bicyclo[1.1.1]pentan-1-yl}-4-hydroxy-7-(trifluoromethyl)-3,4-dihydro-2H-1-benzopyran-2-carboxamide (Compound 243)

The methodologies described in Example 123 substituting Example 131 for Example 214 gave the title compound. ¹H NMR (500 MHz, DMSO-d₆) δ ppm 8.74 (s, 1H), 8.73 (s, 1H), 7.61 (d, J=8.1 Hz, 1H), 7.50 (t, J=8.9 Hz, 1H), 7.27 (dd, J=8.1, 1.8 Hz, 1H), 7.21 (d, J=1.8 Hz, 1H), 7.08 (dd, J=11.3, 2.9 Hz, 1H), 6.89-6.83 (m, 1H), 4.87 (dd, J=10.8, 5.8 Hz, 1H), 4.70 (dd, J=12.0, 2.4 Hz, 1H), 4.49 (s, 2H), 2.41 (ddd, J=12.9, 5.8, 2.5 Hz, 1H), 2.29 (s, 6H), 1.71 (td, J=12.6, 10.9 Hz, 1H); MS (APCI⁺) m/z 511 (M−H₂O+H)⁺.

### Example 145: (2R,4S)-6-chloro-N-{3-[2-(4-chloro-3-fluorophenoxy)acetamido]bicyclo[1.1.1]pentan-1-yl}-4-[(methanesulfonyl)amino]-3,4-dihydro-2H-1-benzopyran-2-carboxamide (Compound 244)

Example 120 was purified by chiral SFC (supercritical fluid chromatography) using a Daicel CHIRALPAK® IC column eluting with 100% CH₃OH in CO₂ with a flow rate of 32 g/minute and back pressure of 100 bar to give the title compound (second isomer eluted out of the column, 0.017 g, 0.030 mmol, 17% yield). The stereochemistry of this title compound was arbitrarily assigned (This compound is the diastereomer of Example 146.). ¹H NMR (400 MHz, DMSO-d₆) δ ppm 8.82 (s, 1H), 8.76 (s, 1H), 7.84 (s, 1H), 7.49 (t, J=8.9 Hz, 1H), 7.35 (d, J=2.7 Hz, 1H), 7.28 (dd, J=8.8, 2.7 Hz, 1H), 7.07 (dd, J=11.4, 2.9 Hz, 1H), 6.95 (d, J=8.8 Hz, 1H), 6.85 (ddd, J=8.9, 2.9, 1.2 Hz, 1H), 4.60 (dd, J=10.5, 2.7 Hz, 1H), 4.53 (d, J=4.2 Hz, 1H), 4.48 (s, 2H), 3.09 (s, 3H), 2.28 (s, 6H), 2.29-2.20 (m, 1H), 2.03 (ddd, J=14.5, 10.7, 4.8 Hz, 1H); MS (APCI⁺) m/z 572 (M+H)⁺.

### Example 146: (2S,4R)-6-chloro-N-{3-[2-(4-chloro-3-fluorophenoxy)acetamido]bicyclo[1.1.1]pentan-1-yl}-4-[(methanesulfonyl)amino]-3,4-dihydro-2H-1-benzopyran-2-carboxamide (Compound 245)

Example 120 was purified by chiral SFC (supercritical fluid chromatography) using a Daicel CHIRALPAK® IC column eluting with 100% CH₃OH in CO₂ with a flow rate of 32 g/minute and back pressure of 100 bar to give the title compound (third isomer eluted out of the column, 0.040 g, 0.070 mmol, 39% yield). The stereochemistry of this title compound was arbitrarily assigned (This compound is the diastereomer of Example 145.). ¹H NMR (400 MHz, DMSO-d₆) δ ppm 8.76 (s, 1H), 8.73 (s, 1H), 7.67 (d, J=8.5 Hz, 1H), 7.50 (t, J=8.9 Hz, 1H), 7.39 (dd, J=2.7, 1.0 Hz, 1H), 7.24 (dd, J=8.7, 2.6 Hz, 1H), 7.08 (dd, J=11.4, 2.8 Hz, 1H), 6.91 (d, J=8.7 Hz, 1H), 6.86 (ddd, J=9.0, 2.8, 1.2 Hz, 1H), 4.74 (s, 1H), 4.65 (dd, J=12.0, 2.2 Hz, 1H), 4.48 (s, 2H), 3.10 (s, 3H), 2.51-2.39 (m, 1H), 2.29 (s, 6H), 1.82 (q, J=12.1 Hz, 1H); MS (APCI⁺) m/z 572 (M+H)⁺.

### Example 147: 7-chloro-N-{3-[2-(4-chloro-3-fluorophenoxy)acetamido]bicyclo[1.1.1]pentan-1-yl}-6-fluoro-4-hydroxy-3,4-dihydro-2H-1-benzopyran-2-carboxamide (Compound 246)

The methodologies described in Example 123 substituting Example 133 for Example 214 gave the title compound. ¹H NMR (400 MHz, DMSO-d₆) δ ppm 8.72 (s, 1H), 8.67 (s, 1H), 7.50 (t, J=8.9 Hz, 1H), 7.31 (dd, J=9.9, 1.0 Hz, 1H), 7.13-7.04 (m, 2H), 6.86 (ddd, J=9.0, 2.9, 1.2 Hz, 1H), 5.77 (s, 1H), 4.79 (dd, J=10.7, 5.8 Hz, 1H), 4.63 (dd, J=12.0, 2.4 Hz, 1H), 4.48 (s, 2H), 2.36 (ddd, J=13.1, 5.9, 2.5 Hz, 1H), 2.28 (s, 6H), 1.68 (td, J=12.4, 10.8 Hz, 1H); MS (APCI⁺) m/z 495 (M−H₂O+H)⁺.

### Example 148: 7-chloro-N-{3-[2-(4-chloro-3-fluorophenoxy)acetamido]bicyclo[1.1.1]pentan-1-yl}-4-hydroxy-3,4-dihydro-2H-1-benzopyran-2-carboxamide (Compound 247)

The methodologies described in Example 123 substituting Example 136 for Example 214 gave the title compound. ¹H NMR (400 MHz, DMSO-d₆) δ ppm 8.72 (s, 1H), 8.67 (s, 1H), 7.50 (t, J=8.9 Hz, 1H), 7.40 (dd, J=8.2, 1.0 Hz, 1H), 7.08 (dd, J=11.4, 2.8 Hz, 1H), 6.98 (dd, J=8.2, 2.1 Hz, 1H), 6.94 (d, J=2.1 Hz, 1H), 6.86 (ddd, J=8.9, 2.8, 1.2 Hz, 1H), 4.78 (dd, J=10.5, 5.7 Hz, 1H), 4.63 (dd, J=11.9, 2.4 Hz, 1H), 4.48 (s, 2H), 2.35 (ddd, J=12.9, 5.8, 2.5 Hz, 1H), 2.29 (s, 6H), 1.76-1.63 (m, 1H); MS (APCI⁺) m/z 477 (M−H₂O+H)⁺.

### Example 149: N-{3-[2-(4-chloro-3-fluorophenoxy)acetamido]bicyclo[1.1.1]pentan-1-yl}-6,7-difluoro-4-oxo-3,4-dihydro-2H-1-benzopyran-2-carboxamide (Compound 248)

The reaction and purification conditions described in Example 23C substituting the product of Example 108C for 2,3-dihydrobenzo[b][1,4]dioxine-2-carboxylic acid gave the title compound. ¹H NMR (500 MHz, DMSO-d₆) δ ppm 8.93 (s, 1H), 8.72 (s, 1H), 7.68 (dd, J=10.3, 9.1 Hz, 1H), 7.49 (t, J=8.9 Hz, 1H), 7.26 (dd, J=11.4, 6.5 Hz, 1H), 7.07 (dd, J=11.4, 2.9 Hz, 1H), 6.85 (ddd, J=9.0, 2.8, 1.2 Hz, 1H), 5.11 (t, J=7.1 Hz, 1H), 4.47 (s, 2H), 2.95 (d, J=7.0 Hz, 2H), 2.26 (s, 6H); MS (ESI⁺) m/z 495 (M+H)⁺.

### Example 150: rac-(2R,4R)—N-{3-[2-(4-chloro-3-fluorophenoxy)acetamido]bicyclo[1.1.1]pentan-1-yl}-6,7-difluoro-4-hydroxy-3,4-dihydro-2H-1-benzopyran-2-carboxamide (Compound 249)

The reaction and purification conditions described in Example 108F substituting the product of Example 149 for the product of Example 108E gave the title compound. ¹H NMR (500 MHz, DMSO-d₆) δ ppm 8.72 (s, 1H), 8.66 (s, 1H), 7.50 (t, J=8.9 Hz, 1H), 7.38-7.28 (m, 1H), 7.08 (dd, J=11.4, 2.8 Hz, 1H), 6.92 (dd, J=11.9, 7.0 Hz, 1H), 6.86 (ddd, J=9.0, 2.9, 1.2 Hz, 1H), 5.71 (d, J=5.8 Hz, 1H), 4.77 (dt, J=10.9, 4.9 Hz, 1H), 4.62 (dd, J=12.0, 2.4 Hz, 1H), 4.48 (s, 2H), 2.34 (ddd, J=13.0, 5.9, 2.4 Hz, 1H), 2.28 (s, 6H), 1.76-1.62 (m, 1H); MS (ESI⁻) m/z 495 (M−H)⁻.

### Example 151: rac-(2R,4S)-6-chloro-N-{3-[2-(4-chloro-3-fluorophenoxy)acetamido]bicyclo[1.1.1]pentan-1-yl}-4-hydroxy-2-methyl-3,4-dihydro-2H-1-benzopyran-2-carboxamide (Compound 250)

The methodologies described in Example 123 substituting Example 239 for Example 214 gave the title compound. The exact stereochemistry was arbitrarily assigned (This is the diastereomer of Example 152 and the first eluting compound). ¹H NMR (500 MHz, DMSO-d₆) δ ppm 8.69 (s, 1H), 8.45 (s, 1H), 7.49 (t, J=8.9 Hz, 1H), 7.34 (dd, J=2.7, 1.0 Hz, 1H), 7.19 (dd, J=8.7, 2.7 Hz, 1H), 7.06 (dd, J=11.3, 2.9 Hz, 1H), 6.88-6.82 (m, 2H), 5.68 (s, 1H), 4.45 (s, 2H), 4.39 (dd, J=10.4, 5.6 Hz, 1H), 2.56 (dd, J=13.1, 5.6 Hz, 1H), 2.19 (d, J=1.4 Hz, 6H), 1.63 (dd, J=13.1, 10.5 Hz, 1H), 1.47 (s, 3H); MS (APCI⁺) m/z 509 (M+H)⁺.

### Example 152: rac-(2R,4R)-6-chloro-N-{3-[2-(4-chloro-3-fluorophenoxy)acetamido]bicyclo[1.1.1]pentan-1-yl}-4-hydroxy-2-methyl-3,4-dihydro-2H-1-benzopyran-2-carboxamide (Compound 251)

The methodologies described in Example 123 substituting Example 239 for Example 214 gave the title compound. The exact stereochemistry was arbitrarily assigned (This is the diastereomer of Example 151 and second eluting compound). ¹H NMR (400 MHz, DMSO-d₆) δ ppm 8.70 (s, 1H), 8.49 (s, 1H), 7.49 (t, J=8.9 Hz, 1H), 7.39 (d, J=2.6 Hz, 1H), 7.22 (dd, J=8.7, 2.7 Hz, 1H), 7.07 (dd, J=11.4, 2.8 Hz, 1H), 6.93 (d, J=8.8 Hz, 1H), 6.85 (ddd, J=9.0, 2.9, 1.2 Hz, 1H), 5.54 (s, 1H), 4.72 (dd, J=10.0, 5.8 Hz, 1H), 4.47 (s, 2H), 2.26 (s, 6H), 2.21 (dd, J=13.2, 5.7 Hz, 2H), 1.79 (dd, J=13.3, 10.0 Hz, 1H), 1.35 (s, 3H), 1.18 (t, J=7.3 Hz, 1H); MS (APCI⁺) m/z 509 (M+H)⁺.

### Example 153: (2R,4R)—N-{3-[2-(4-chloro-3-fluorophenoxy)acetamido]bicyclo[1.1.1]pentan-1-yl}-6,7-difluoro-4-hydroxy-3,4-dihydro-2H-1-benzopyran-2-carboxamide (Compound 252)

### Example 153A: (2R)—N-{3-[2-(4-chloro-3-fluorophenoxy)acetamido]bicyclo[1.1.1]pentan-1-yl}-6,7-difluoro-4-oxo-3,4-dihydro-2H-1-benzopyran-2-carboxamide

The product of Example 149 was purified by preparative chiral HPLC [Daicel CHIRALPAK® AD-H 5 μm column, 20×250 mm, flow rate 5 mL/minute, 60% ethanol in heptane (isocratic gradient)] to give the title compound as the earlier eluting fraction. MS (ESI⁺) m/z 495 (M+H)⁺.

### Example 153B: (2R,4R)—N-{3-[2-(4-chloro-3-fluorophenoxy)acetamido]bicyclo[1.1.1]pentan-1-yl}-6,7-difluoro-4-hydroxy-3,4-dihydro-2H-1-benzopyran-2-carboxamide

The reaction and purification conditions described in Example 108F substituting the product of Example 153A for the product of Example 108E gave the title compound. ¹H NMR (501 MHz, DMSO-d₆) δ ppm 8.72 (s, 1H), 8.66 (s, 1H), 7.50 (t, J=8.9 Hz, 1H), 7.33 (ddd, J=11.4, 9.2, 1.0 Hz, 1H), 7.08 (dd, J=11.4, 2.8 Hz, 1H), 6.92 (dd, J=11.9, 7.0 Hz, 1H), 6.86 (ddd, J=9.0, 2.9, 1.2 Hz, 1H), 5.71 (d, J=4.3 Hz, 1H), 4.81-4.73 (m, 1H), 4.62 (dd, J=11.9, 2.3 Hz, 1H), 4.48 (s, 2H), 2.34 (ddd, J=12.9, 5.8, 2.4 Hz, 1H), 2.28 (s, 6H), 1.76-1.62 (m, 1H); MS (ESI⁻) m/z 495 (M−H)⁻.

### Example 154: (2S,4S)—N-{3-[2-(4-chloro-3-fluorophenoxy)acetamido]bicyclo[1.1.1]pentan-1-yl}-6,7-difluoro-4-hydroxy-3,4-dihydro-2H-1-benzopyran-2-carboxamide (Compound 253)

### Example 154A: (2S)—N-{3-[2-(4-chloro-3-fluorophenoxy)acetamido]bicyclo[1.1.1]pentan-1-yl}-6,7-difluoro-4-oxo-3,4-dihydro-2H-1-benzopyran-2-carboxamide

The product of Example 149 was purified by preparative chiral HPLC [Daicel CHIRALPAK® AD-H 5 μm column, 20×250 mm, flow rate 5 mL/minute, 60% ethanol in heptane (isocratic gradient)] to give the title compound as the later eluting fraction. MS (ESI⁺) m/z 495 (M+H)⁺.

### Example 154B: (2S,4S)—N-{3-[2-(4-chloro-3-fluorophenoxy)acetamido]bicyclo[1.1.1]pentan-1-yl}-6,7-difluoro-4-hydroxy-3,4-dihydro-2H-1-benzopyran-2-carboxamide

The reaction and purification conditions described in Example 108F substituting the product of Example 154A for the product of Example 108E gave the title compound. ¹H NMR (500 MHz, DMSO-d₆) δ ppm 8.73 (s, 1H), 8.68 (s, 1H), 7.50 (t, J=8.9 Hz, 1H), 7.33 (ddd, J=11.4, 9.3, 0.9 Hz, 1H), 7.08 (dd, J=11.4, 2.8 Hz, 1H), 6.92 (dd, J=11.8, 7.0 Hz, 1H), 6.86 (ddd, J=8.9, 2.9, 1.2 Hz, 1H), 5.72 (d, J=5.8 Hz, 1H), 4.77 (dt, J=11.1, 5.7 Hz, 1H), 4.62 (dd, J=12.0, 2.3 Hz, 1H), 4.48 (s, 2H), 2.34 (ddd, J=13.0, 5.9, 2.4 Hz, 1H), 2.28 (s, 6H), 1.69 (ddd, J=12.8, 12.0, 10.7 Hz, 1H); MS (ESI⁻) m/z 495 (M−H)⁻.

### Example 155: N-{3-[2-(4-chloro-3-fluorophenoxy)acetamido]bicyclo[1.1.1]pentan-1-yl}-4-hydroxy-6-methyl-3,4-dihydro-2H-1-benzopyran-2-carboxamide (Compound 254)

The methodologies described in Example 123 substituting Example 242 for Example 214 gave the title compound. ¹H NMR (400 MHz, DMSO-d₆) δ ppm 8.71 (s, 1H), 8.60 (s, 1H), 7.50 (t, J=8.9 Hz, 1H), 7.21 (d, J=2.2 Hz, 1H), 7.08 (dd, J=11.3, 2.9 Hz, 1H), 6.94 (dd, J=8.3, 2.3 Hz, 1H), 6.86 (ddd, J=8.9, 2.9, 1.2 Hz, 1H), 6.73 (d, J=8.2 Hz, 1H), 4.77 (dd, J=10.6, 5.9 Hz, 1H), 4.56-4.47 (m, 1H), 4.48 (s, 2H), 2.32 (ddd, J=12.8, 6.0, 2.4 Hz, 1H), 2.28 (s, 6H), 2.22 (s, 3H), 1.69 (td, J=12.4, 10.7 Hz, 1H); MS (APCI⁺) m/z 456 (M−H₂O+H)⁺.

### Example 156: (2R)-6-chloro-N-{4-[2-(4-chloro-3-fluorophenoxy)acetamido]bicyclo[2.1.1]hexan-1-yl}-4-oxo-3,4-dihydro-2H-1-benzopyran-2-carboxamide (Compound 255)

The reaction and purification conditions described in Example 108E substituting N-(4-aminobicyclo[2.1.1]hexan-1-yl)-2-(4-chloro-3-fluorophenoxy)acetamide (prepared as described in International Patent Publication WO2017/193034 A1) for the product of Example 108A, and the product of Example 124A for the product of Example 108D gave the title compound. ¹H NMR (400 MHz, DMSO-d₆) δ ppm 8.70 (s, 1H), 8.48 (s, 1H), 7.67-7.60 (m, 2H), 7.49 (t, J=8.9 Hz, 1H), 7.18 (dd, J=8.7, 0.6 Hz, 1H), 7.06 (dd, J=11.4, 2.8 Hz, 1H), 6.85 (ddd, J=8.9, 2.9, 1.2 Hz, 1H), 5.09 (t, J=7.0 Hz, 1H), 4.47 (s, 2H), 2.95 (d, J=6.9 Hz, 2H), 2.09-2.03 (m, 2H), 1.82-1.74 (m, 6H); MS (ESI⁺) m/z 524 (M+NH₄)⁺.

### Example 157: (2R,4R)-6-chloro-N-{4-[2-(4-chloro-3-fluorophenoxy)acetamido]bicyclo[2.1.1]hexan-1-yl}-4-hydroxy-3,4-dihydro-2H-1-benzopyran-2-carboxamide (Compound 256)

The reaction and purification conditions described in Example 108F substituting the product of Example 156 for the product of Example 108E gave the title compound. ¹H NMR (500 MHz, DMSO-d₆) δ ppm δ ppm 8.49 (s, 1H), 8.41 (s, 1H), 7.50 (t, J=8.8 Hz, 1H), 7.41-7.35 (m, 1H), 7.19 (dd, J=8.7, 2.7 Hz, 1H), 7.07 (dd, J=11.4, 2.8 Hz, 1H), 6.92-6.81 (m, 2H), 5.69 (d, J=5.7 Hz, 1H), 4.84-4.77 (m, 1H), 4.59 (dd, J=12.0, 2.2 Hz, 1H), 4.48 (s, 2H), 2.53-2.50 (m, 1H), 2.34 (ddd, J=12.9, 6.0, 2.3 Hz, 1H), 2.11-2.06 (m, 2H), 1.88-1.79 (m, 5H), 1.72 (td, J=12.5, 10.9 Hz, 1H); MS (ESI⁻) m/z 507 (M−H)⁻.

### Example 158: (2S)-5-chloro-N-{3-[2-(4-chloro-3-fluorophenoxy)acetamido]bicyclo[1.1.1]pentan-1-yl}-2-methyl-2,3-dihydro-1-benzofuran-2-carboxamide (Compound 257)

The methodologies described in Example 122 substituting (S)-5-chloro-2-methyl-2,3-dihydrobenzofuran-2-carboxylic acid (U.S. Pat. Appl. Publ. (2017), US 20170305891 Al) for 6,7-dimethoxy-4-oxo-4H-chromene-2-carboxylic acid gave the title compound. ¹H NMR (400 MHz, DMSO-d₆) δ ppm 8.69 (s, 1H), 8.64 (s, 1H), 7.49 (t, J=8.9 Hz, 1H), 7.25 (d, J=2.3 Hz, 1H), 7.17 (dd, J=8.5, 2.4 Hz, 1H), 7.06 (dd, J=11.4, 2.9 Hz, 1H), 6.84 (dd, J=8.8, 4.4 Hz, 2H), 4.46 (s, 2H), 3.08 (d, J=16.6 Hz, 1H), 2.23 (s, 6H), 1.52 (s, 3H); MS (APCI⁺) m/z 479 (M+H)⁺.

### Example 159: (2R)-5-chloro-N-{3-[2-(4-chloro-3-fluorophenoxy)acetamido]bicyclo[1.1.1]pentan-1-yl}-2-methyl-2,3-dihydro-1-benzofuran-2-carboxamide (Compound 258)

The methodologies described in Example 122 substituting (R)-5-chloro-2-methyl-2,3-dihydrobenzofuran-2-carboxylic acid (U.S. Pat. Appl. Publ. (2017), US 20170305891 Al) for 6,7-dimethoxy-4-oxo-4H-chromene-2-carboxylic acid gave the title compound. ¹H NMR (500 MHz, DMSO-d₆) δ ppm 8.71 (s, 1H), 8.67 (s, 1H), 7.49 (t, J=8.9 Hz, 1H), 7.25 (d, J=2.3 Hz, 1H), 7.17 (dd, J=8.5, 2.3 Hz, 1H), 7.07 (dd, J=11.4, 2.8 Hz, 1H), 6.84 (dd, J=7.7, 2.9 Hz, 2H), 4.46 (s, 2H), 3.08 (d, J=16.6 Hz, 1H), 2.23 (s, 6H), 2.07 (s, 1H), 1.52 (s, 3H); MS (APCI⁺) m/z 479 (M+H)⁺.

### Example 160: (2S)-5-chloro-N-{3-[2-(4-chloro-3-fluorophenoxy)acetamido]bicyclo[1.1.1]pentan-1-yl}-2-methyl-3-oxo-2,3-dihydro-1-benzofuran-2-carboxamide (Compound 259)

Modifying a reported benzylic oxidation procedure (U.S. Pat. Appl. Publ. (2004), US 20040224994 A1) to a mixture of Example 158 (0.144 g, 0.300 mmol) in CH₃CN (1.15 mL) and H₂O (1.15 mL) was added potassium persulfate (0.203 g, 0.749 mmol) and copper(II) sulfate pentahydrate (0.075 g, 0.30 mmol). The reaction mixture was heated to 80° C. for 20 minutes and then to 50° C. overnight. Then the reaction mixture was cooled to ambient temperature, diluted with H₂O (1 mL) and extracted with dichloromethane (3×5 mL). The combined organic extracts were dried over Na₂SO₄ and concentrated. The crude material was diluted with N,N-dimethylformamide, filtered, and purified by preparative HPLC (Waters XBridge™ C18 5 μm OBD column, 30×100 mm, flow rate 40 mL/minute, 5-100% gradient of acetonitrile in 0.1% trifluoroacetic acid/water) to give the title compound (0.030 g, 0.061 mmol, 20% yield) and Example 163 (11% yield). ¹H NMR (400 MHz, DMSO-d₆) δ ppm 8.73 (s, 1H), 8.68 (s, 1H), 7.83 (dd, J=8.9, 2.4 Hz, 1H), 7.72 (d, J=2.3 Hz, 1H), 7.48 (t, J=8.9 Hz, 1H), 7.39 (d, J=8.9 Hz, 1H), 7.06 (dd, J=11.4, 2.9 Hz, 1H), 6.84 (ddd, J=9.0, 2.9, 1.2 Hz, 1H), 4.46 (s, 2H), 2.21 (s, 6H), 1.60 (s, 3H); MS (APCI⁺) m/z 493 (M+H)⁺.

### Example 161: (2R,4S)-6-chloro-N-{4-[2-(4-chloro-3-fluorophenoxy)acetamido]bicyclo[2.1.1]hexan-1-yl}-4-hydroxy-3,4-dihydro-2H-1-benzopyran-2-carboxamide (Compound 260)

The product of Example 157 (12 mg, 0.024 mmol) was dissolved in trifluoroacetic acid (0.5 mL) and stirred at 35° C. for 2 hours. The residue was concentrated under reduced pressure, and the residue was first purified by preparative HPLC [YMC TriArt™ C18 Hybrid 5 μm column, 50×100 mm, flow rate 140 mL/minute, 5-100% gradient of acetonitrile in buffer (0.025 M aqueous ammonium bicarbonate, adjusted to pH 10 with ammonium hydroxide)]. Fractions containing the desired product were combined and concentrated, and further purified by preparative chiral HPLC [Daicel CHIRALPAK® AD-H 5 μm column, 20×250 mm, flow rate 10 mL/minute, 40% ethanol in heptane (isocratic gradient)] to give the title compound (11 mg, 0.022 mmol, 92% yield). ¹H NMR (500 MHz, DMSO-d₆) δ ppm 8.48 (s, 1H), 8.47 (s, 1H), 7.49 (t, J=8.9 Hz, 1H), 7.31 (d, J=2.7 Hz, 1H), 7.24 (dd, J=8.8, 2.7 Hz, 1H), 7.07 (dd, J=11.4, 2.8 Hz, 1H), 6.93 (d, J=8.7 Hz, 1H), 6.85 (ddd, J=9.0, 2.9, 1.2 Hz, 1H), 5.59 (d, J=4.6 Hz, 1H), 4.63-4.52 (m, 1H), 4.48 (s, 2H), 2.12-2.03 (m, 3H), 1.92 (ddd, J=14.1, 10.7, 3.8 Hz, 1H), 1.86-1.79 (m, 6H), 1.06 (t, J=7.0 Hz, 1H); MS (ESI⁻) m/z 507 (M−H)⁻.

### Example 162: 5-chloro-N-{3-[2-(4-chloro-3-fluorophenoxy)acetamido]bicyclo[1.1.1]pentan-1-yl}-2,3-dihydro-1H-indole-2-carboxamide (Compound 261)

The methodologies described in Example 122 substituting 5-chloroindoline-2-carboxylic acid hydrochloride hydrate for 6,7-dimethoxy-4-oxo-4H-chromene-2-carboxylic acid gave the title compound. ¹H NMR (400 MHz, DMSO-d₆) δ ppm 8.69 (s, 1H), 8.46 (s, 1H), 7.49 (t, J=8.9 Hz, 1H), 7.07 (dd, J=11.4, 2.8 Hz, 1H), 7.02 (d, J=2.2 Hz, 1H), 6.95 (dd, J=8.3, 2.3 Hz, 1H), 6.85 (ddd, J=9.0, 2.9, 1.2 Hz, 1H), 6.51 (d, J=8.2 Hz, 1H), 4.47 (s, 2H), 4.17 (dd, J=10.5, 7.3 Hz, 1H), 3.25 (dd, J=16.6, 10.5 Hz, 1H), 2.93 (dd, J=16.5, 7.3 Hz, 1H), 2.24 (s, 6H); MS (APCI⁺) m/z 464 (M+H)⁺.

### Example 163: (2S)-5-chloro-N-{3-[2-(4-chloro-3-fluorophenoxy)acetamido]bicyclo[1.1.1]pentan-1-yl}-3-hydroxy-2-methyl-2,3-dihydro-1-benzofuran-2-carboxamide (Compound 262)

Modifying a reported benzylic oxidation procedure (U.S. Pat. Appl. Publ. (2004), US 20040224994 A1) to a mixture of Example 158 (0.144 g, 0.300 mmol) in CH₃CN (1.15 mL) and H₂O (1.15 mL) was added potassium persulfate (0.203 g, 0.749 mmol) and copper(II) sulfate pentahydrate (0.075 g, 0.30 mmol). The reaction mixture was heated to 80° C. for 20 minutes and then to 50° C. overnight. Then the reaction mixture was cooled to ambient temperature, diluted with H₂O (1 mL) and extracted with dichloromethane (3×5 mL). The combined organic extracts were dried over Na₂SO₄ and concentrated. The crude material was diluted with N,N-dimethylformamide, filtered, and purified by preparative HPLC (Waters XBridge™ C18 5 μm OBD column, 30×100 mm, flow rate 40 mL/minute, 5-100% gradient of acetonitrile in 0.1% trifluoroacetic acid/water) to give the title compound (0.016 g, 0.032 mmol, 11% yield) and Example 160 (20% yield). ¹H NMR (500 MHz, DMSO-d₆, dr 1.4:1) δ ppm 8.69 (s, 0.7H), 8.68 (s, 1H), 8.63 (s, 1H), 8.34 (s, 0.7H), 7.49 (td, J=8.9, 4.9 Hz, 2H), 7.36 (t, J=2.2 Hz, 2H), 7.30 (td, J=8.1, 2.4 Hz, 2H), 7.07 (ddd, J=11.4, 6.3, 2.9 Hz, 2H), 6.91 (d, J=8.5 Hz, 2H), 6.88-6.81 (m, 2H), 5.91 (s, 2H), 5.14 (s, 1H), 4.81 (s, 0.7H), 4.47 (s, 1.4H), 4.45 (s, 2H), 2.25 (s, 4H), 2.19 (d, J=2.3 Hz, 6H), 1.44 (s, 3H), 1.35 (s, 2H); MS (APCI⁺) m/z 495 (M+H)⁺.

### Example 164: (2R)-5-chloro-N-{3-[2-(4-chloro-3-fluorophenoxy)acetamido]bicyclo[1.1.1]pentan-1-yl}-3-hydroxy-2-methyl-2,3-dihydro-1-benzofuran-2-carboxamide (Compound 263)

The methodologies described in Example 123 substituting Example 221 for Example 214 and increasing the reaction time to 3 hours gave the title compound. ¹H NMR (400 MHz, DMSO-d₆, dr 5:1) δ ppm 8.68 (s, 0.2H), 8.67 (s, 1H), 8.62 (s, 1H), 8.32 (s, 0.2H), 7.49 (td, J=8.9, 3.9 Hz, 1.2H), 7.36 (d, J=2.4 Hz, 1H), 7.29 (ddd, J=8.7, 6.4, 2.4 Hz, 1.2H), 7.06 (ddd, J=11.3, 5.0, 2.8 Hz, 1.2H), 6.91 (d, J=8.6 Hz, 1.2H), 6.88-6.80 (m, 1.2H), 5.91 (m, 0.2H), 5.89 (d, J=7.1 Hz, 1H), 5.14 (d, J=7.1 Hz, 1H), 4.81 (d, J=6.0 Hz, 0.2H), 4.47 (s, 0.4H), 4.45 (s, 2H), 2.25 (s, 1.3H), 2.19 (d, J=1.3 Hz, 6H), 1.44 (s, 3H), 1.35 (s, 0.6H); MS (APCI⁺) m/z 495 (M+H)⁺.

### Example 165: (2R,4R)-6-chloro-N-{3-[2-(4-chloro-3-fluorophenoxy)acetamido]bicyclo[1.1.1]pentan-1-yl}-7-fluoro-4-hydroxy-3,4-dihydro-2H-1-benzopyran-2-carboxamide (Compound 264)

Example 135 was purified by chiral SFC (supercritical fluid chromatography) using a Regis® Technologies, Inc. Whelk-O® (S, S) column eluting with 100% CH₃OH in CO₂ with a flow rate of 70 g/minute and back pressure of 100 bar to give the title compound (first isomer eluted out of the column, 0.023 g, 0.045 mmol, 46% yield). The stereochemistry of this title compound was arbitrarily assigned (This compound is the enantiomer of Examples 166.). ¹H NMR (400 MHz, DMSO-d₆) δ ppm 8.72 (s, 1H), 8.70 (s, 1H), 7.54-7.44 (m, 2H), 7.08 (dd, J=11.4, 2.8 Hz, 1H), 6.92 (d, J=10.5 Hz, 1H), 6.86 (ddd, J=9.0, 2.9, 1.2 Hz, 1H), 5.75 (s, 1H), 4.78 (dd, J=10.5, 5.8 Hz, 1H), 4.65 (dd, J=11.9, 2.4 Hz, 1H), 4.48 (s, 2H), 2.34 (ddd, J=13.0, 5.8, 2.5 Hz, 1H), 2.28 (s, 6H), 1.70 (td, J=12.8, 12.3, 10.7 Hz, 1H); MS (APCI⁺) m/z 495 (M−H₂O+H)⁺.

### Example 166: (2S,4S)-6-chloro-N-{3-[2-(4-chloro-3-fluorophenoxy)acetamido]bicyclo[1.1.1]pentan-1-yl}-7-fluoro-4-hydroxy-3,4-dihydro-2H-1-benzopyran-2-carboxamide (Compound 265)

Example 135 was purified by chiral SFC (supercritical fluid chromatography) using a Regis® Technologies, Inc. Whelk-O® (S, S) column eluting with 100% CH₃OH in CO₂ with a flow rate of 70 g/minute and back pressure of 100 bar to give the title compound (second isomer eluted out of the column, 0.020 g, 0.039 mmol, 40% yield). The stereochemistry of this title compound was arbitrarily assigned (This compound is the enantiomer of Examples 165.). ¹H NMR (400 MHz, DMSO-d₆) δ ppm 8.73 (s, 1H), 8.70 (s, 1H), 7.54-7.44 (m, 2H), 7.08 (dd, J=11.4, 2.8 Hz, 1H), 6.92 (d, J=10.5 Hz, 1H), 6.86 (ddd, J=9.0, 2.9, 1.2 Hz, 1H), 5.74 (d, J=4.7 Hz, 1H), 4.78 (s, 1H), 4.65 (dd, J=11.8, 2.3 Hz, 1H), 4.48 (s, 2H), 2.35 (ddd, J=13.0, 5.7, 2.4 Hz, 1H), 2.28 (s, 6H), 1.77-1.64 (m, 1H); MS (APCI⁺) m/z 495 (M−H₂O+H)⁺.

### Example 167: 5-chloro-N-{3-[2-(4-chloro-3-fluorophenoxy)acetamido]bicyclo[1.1.1]pentan-1-yl}-1-methyl-2,3-dihydro-1H-indole-2-carboxamide (Compound 266)

To a solution of the product of Example 162 (0.041 g, 0.088 mmol) in 2.4 weight % sodium acetate trihydrate and 3.6 weight % acetic acid in methanol (0.88 mL) was added formaldehyde (6.9 μL, 0.092 mmol), followed by sodium cyanoborohydride (0.014 g, 0.22 mmol). The reaction mixture stirred at ambient temperature for 4 hours and was concentrated. The residue was purified by preparative HPLC (Phenomenex® Luna® C18(2) 10 μm 100 Å AXIA™ column (250 mm×50 mm). A 30-100% gradient of acetonitrile (A) and 0.1% trifluoroacetic acid in water (B) was used over 25 minutes, at a flow rate of 50 mL/minute) to give the title compound (0.019 g, 0.40 mmol, 45% yield). ¹H NMR (500 MHz, DMSO-d₆) δ ppm 8.74 (s, 1H), 8.68 (s, 1H), 7.50 (t, J=8.9 Hz, 1H), 7.11-7.00 (m, 3H), 6.87 (ddd, J=9.0, 2.9, 1.2 Hz, 1H), 6.48 (d, J=8.3 Hz, 1H), 4.49 (s, 2H), 3.86 (t, J=9.5 Hz, 1H), 3.29-3.17 (m, 1H), 2.86 (ddt, J=16.2, 9.3, 1.1 Hz, 1H), 2.67 (s, 3H), 2.28 (s, 6H); MS (APCI⁺) m/z 478 (M+H)⁺.

### Example 168: (2R,4R)—N-{3-[2-(4-chloro-3-fluorophenoxy)acetamido]bicyclo[1.1.1]pentan-1-yl}-4-hydroxy-3,4-dihydro-2H-1-benzopyran-2-carboxamide (Compound 267)

The reaction and purification conditions described in Example 108F substituting the product of Example 169 for the product of Example 108E gave the title compound. ¹H NMR (400 MHz, DMSO-d₆) δ ppm 8.72 (s, 1H), 8.64 (s, 1H), 7.50 (t, J=8.9 Hz, 1H), 7.43-7.38 (m, 1H), 7.17-7.12 (m, 1H), 7.08 (dd, J=11.4, 2.8 Hz, 1H), 6.92 (td, J=7.4, 1.2 Hz, 1H), 6.88-6.82 (m, 2H), 5.51 (br s, 1H), 4.81 (dd, J=10.6, 5.9 Hz, 1H), 4.56 (dd, J=12.0, 2.2 Hz, 1H), 4.48 (s, 2H), 2.35 (ddd, J=12.8, 5.9, 2.2 Hz, 1H), 2.29 (s, 6H), 1.78-1.64 (m, 1H); MS (APCI⁺) m/z 461 (M+H)⁺.

### Example 169: (2R)—N-{3-[2-(4-chloro-3-fluorophenoxy)acetamido]bicyclo[1.1.1]pentan-1-yl}-4-oxo-3,4-dihydro-2H-1-benzopyran-2-carboxamide (Compound 268)

### Example 169A: (R)-4-oxochroman-2-carboxylic Acid

4-Oxochroman-2-carboxylic acid (Enamine) was purified by preparative chiral HPLC [Daicel CHIRALPAK® AD-H 5 μm column, 20×250 mm, flow rate 6 mL/minute, 80% ethanol in heptane (isocratic gradient)] to give the title compound as the earlier eluting fraction. MS (ESI⁺) m/z 193 (M+H)⁺.

### Example 169B: (2R)—N-{3-[2-(4-chloro-3-fluorophenoxy)acetamido]bicyclo[1.1.1]pentan-1-yl}-4-oxo-3,4-dihydro-2H-1-benzopyran-2-carboxamide

The reaction and purification conditions described in Example 23C substituting the product of Example 169A for 2,3-dihydrobenzo[b][1,4]dioxine-2-carboxylic acid gave the title compound. ¹H NMR (400 MHz, DMSO-d₆) δ ppm 8.91 (s, 1H), 8.72 (s, 1H), 7.73 (dd, J=7.8, 1.7 Hz, 1H), 7.60 (ddd, J=8.6, 7.2, 1.8 Hz, 1H), 7.49 (t, J=8.9 Hz, 1H), 7.15-7.04 (m, 3H), 6.85 (ddd, J=9.0, 2.9, 1.2 Hz, 1H), 5.04 (dd, J=9.7, 4.9 Hz, 1H), 4.48 (s, 2H), 3.02-2.81 (m, 2H), 2.27 (s, 6H); MS (APCI⁺) m/z 459 (M+H)⁺.

### Example 170: (2S)—N-{3-[2-(4-chloro-3-fluorophenoxy)acetamido]bicyclo[1.1.1]pentan-1-yl}-4-oxo-3,4-dihydro-2H-1-benzopyran-2-carboxamide (Compound 269)

### Example 170A: (S)-4-oxochroman-2-carboxylic Acid

4-Oxochroman-2-carboxylic acid (Enamine) was purified by preparative chiral HPLC [Daicel CHIRALPAK® AD-H 5 μm column, 20×250 mm, flow rate 6 mL/minute, 80% ethanol in heptane (isocratic gradient)] to give the title compound as the later eluting fraction. MS (ESI⁺) m/z 193 (M+H)⁺.

### Example 170B: (2S)—N-{3-[2-(4-chloro-3-fluorophenoxy)acetamido]bicyclo[1.1.1]pentan-1-yl}-4-oxo-3,4-dihydro-2H-1-benzopyran-2-carboxamide

The reaction and purification conditions described in Example 23C substituting the product of Example 170A for 2,3-dihydrobenzo[b][1,4]dioxine-2-carboxylic acid gave the title compound. ¹H NMR (400 MHz, DMSO-d₆) δ ppm 8.91 (s, 1H), 8.72 (s, 1H), 7.73 (dd, J=7.8, 1.7 Hz, 1H), 7.60 (ddd, J=8.6, 7.2, 1.8 Hz, 1H), 7.49 (t, J=8.9 Hz, 1H), 7.15-7.04 (m, 3H), 6.85 (ddd, J=9.0, 2.9, 1.2 Hz, 1H), 5.04 (dd, J=9.7, 4.9 Hz, 1H), 4.48 (s, 2H), 3.02-2.81 (m, 2H), 2.27 (s, 6H); MS (APCI⁺) m/z 459 (M+H)⁺.

### Example 171: (2S,4S)—N-{3-[2-(4-chloro-3-fluorophenoxy)acetamido]bicyclo[1.1.1]pentan-1-yl}-4-hydroxy-3,4-dihydro-2H-1-benzopyran-2-carboxamide (Compound 270)

The reaction and purification conditions described in Example 108F substituting the product of Example 170B for the product of Example 108E gave the title compound. ¹H NMR (500 MHz, DMSO-d₆) δ ppm 8.73 (s, 1H), 8.65 (s, 1H), 7.50 (t, J=8.9 Hz, 1H), 7.42-7.38 (m, 1H), 7.15 (dddd, J=8.0, 7.2, 1.8, 0.7 Hz, 1H), 7.08 (dd, J=11.4, 2.9 Hz, 1H), 6.92 (td, J=7.5, 1.2 Hz, 1H), 6.88-6.83 (m, 2H), 5.55-5.48 (m, 1H), 4.88-4.77 (m, 1H), 4.56 (dd, J=12.0, 2.2 Hz, 1H), 4.48 (s, 2H), 2.34 (ddd, J=12.8, 6.0, 2.3 Hz, 1H), 2.29 (s, 6H), 1.72 (ddd, J=12.9, 12.0, 10.7 Hz, 1H); MS (APCI⁻) m/z 459 (M−H)⁻.

### Example 172: (2R)—N-{3-[2-(3,4-difluorophenoxy)acetamido]bicyclo[1.1.1]pentan-1-yl}-6,7-difluoro-4-oxo-3,4-dihydro-2H-1-benzopyran-2-carboxamide (Compound 271)

### Example 172A: N-(3-aminobicyclo[1.1.1]pentan-1-yl)-2-(3,4-difluorophenoxy)acetamide

The reaction and purification conditions described in Example 23A through Example 23B substituting 2-(3,4-difluorophenoxy)acetic acid for 2-(4-chloro-3-fluorophenoxy)acetic acid gave the title compound. MS (APCI⁺) m/z 269 (M+H)⁺.

### Example 172B: (2R)—N-{3-[2-(3,4-difluorophenoxy)acetamido]bicyclo[1.1.1]pentan-1-yl}-6,7-difluoro-4-oxo-3,4-dihydro-2H-1-benzopyran-2-carboxamide

The reaction and purification conditions described in Example 108E substituting the product of Example 172A for the product of Example 108A gave the title compound. ¹H NMR (500 MHz, DMSO-d₆) δ ppm 8.94 (s, 1H), 8.71 (s, 1H), 7.69 (dd, J=10.2, 9.1 Hz, 1H), 7.37 (dt, J=10.6, 9.3 Hz, 1H), 7.27 (dd, J=11.3, 6.4 Hz, 1H), 7.09 (ddd, J=12.6, 6.7, 3.0 Hz, 1H), 6.79 (dtd, J=9.1, 3.3, 1.7 Hz, 1H), 5.11 (dd, J=7.5, 6.6 Hz, 1H), 4.44 (s, 2H), 2.97-2.92 (m, 2H), 2.26 (s, 6H); MS (APCI⁺) m/z 479 (M+H)⁺.

### Example 173: (2R,4R)—N-{3-[2-(3,4-difluorophenoxy)acetamido]bicyclo[1.1.1]pentan-1-yl}-6,7-difluoro-4-hydroxy-3,4-dihydro-2H-1-benzopyran-2-carboxamide (Compound 272)

The reaction and purification conditions described in Example 108F substituting the product of Example 172B for the product of Example 108E gave the title compound. ¹H NMR (400 MHz, DMSO-d₆) δ ppm 8.70 (s, 1H), 8.67 (s, 1H), 7.42-7.28 (m, 2H), 7.09 (ddd, J=12.6, 6.8, 3.0 Hz, 1H), 6.92 (dd, J=11.9, 7.0 Hz, 1H), 6.80 (dtd, J=9.1, 3.3, 1.7 Hz, 1H), 5.71 (s, 1H), 4.81-4.72 (m, 1H), 4.62 (dd, J=12.0, 2.3 Hz, 1H), 4.44 (s, 2H), 2.34 (ddd, J=13.0, 5.8, 2.4 Hz, 1H), 2.28 (s, 6H), 1.76-1.62 (m, 1H); MS (APCI⁺) m/z 463 (M−H₂O+H)⁺.

### Example 174: (2S,4S)—N-{3-[2-(3,4-difluorophenoxy)acetamido]bicyclo[1.1.1]pentan-1-yl}-6,7-difluoro-4-hydroxy-3,4-dihydro-2H-1-benzopyran-2-carboxamide (Compound 273)

The reaction and purification conditions described in Example 108F substituting the product of Example 222 for the product of Example 108E gave the title compound. ¹H NMR (400 MHz, DMSO-d₆) δ ppm 8.70 (s, 1H), 8.67 (s, 1H), 7.43-7.28 (m, 2H), 7.09 (ddd, J=12.7, 6.7, 3.0 Hz, 1H), 6.92 (dd, J=11.8, 6.9 Hz, 1H), 6.80 (dtd, J=9.2, 3.3, 1.7 Hz, 1H), 5.71 (s, 1H), 4.77 (dd, J=10.5, 5.9 Hz, 1H), 4.62 (dd, J=12.0, 2.3 Hz, 1H), 4.44 (s, 2H), 2.34 (ddd, J=13.0, 5.9, 2.4 Hz, 1H), 2.28 (s, 6H), 1.76-1.62 (m, 1H); MS (APCI⁺) m/z 463 (M−H₂O+H)⁺.

### Example 175: N-[(2S)-4-{[(2R,4R)-6-chloro-4-hydroxy-3,4-dihydro-2H-1-benzopyran-2-carbonyl]amino}-2-hydroxybicyclo[2.2.2]octan-1-yl]-2-ethyl-1,3-oxazole-5-carboxamide (Compound 274)

### Example 175A: tert-butyl ((S)-4-((R)-6-chloro-4-oxochroman-2-carboxamido)-2-hydroxybicyclo[2.2.2]octan-1-yl)carbamate

The reaction and purification conditions described in Example 108E substituting the product of Example 85C for the product of Example 108A, and the product of Example 124A for the product of Example 108D gave the title compound. MS (ESI⁺) m/z 409 [M−(t-Bu)+H]⁺.

### Example 175B: N-[(2S)-4-{[(2R)-6-chloro-4-oxo-3,4-dihydro-2H-1-benzopyran-2-carbonyl]amino}-2-hydroxybicyclo[2.2.2]octan-1-yl]-2-ethyl-1,3-oxazole-5-carboxamide

The product of Example 175A (20 mg, 0.043 mmol) and trifluoroacetic acid (0.5 mL) were combined and stirred at ambient temperature for 30 minutes and then the mixture was concentrated under reduced pressure. To the residue was added N,N-dimethylformamide (2 mL), triethylamine (0.018 mL) and 2-ethyloxazole-5-carboxylic acid (Enamine, 6.1 mg, 0.043 mmol). While stirring, 1-[bis(dimethylamino)methylene]-1H-1,2,3-triazolo[4,5-b]pyridinium 3-oxid hexafluorophosphate (HATU, 18 mg, 0.047 mmol) was added. After 30 minutes, water (0.2 mL) was added, and the resulting mixture was directly purified by preparative HPLC [Waters XBridge™ C18 5 μm OBD column, 30×100 mm, flow rate 40 mL/minute, 5-100% gradient of acetonitrile in buffer (0.025 M aqueous ammonium bicarbonate, adjusted to pH 10 with ammonium hydroxide)] to give the title compound (12 mg, 0.017 mmol, 40% yield). MS (APCI⁺) m/z 488 (M+H)⁺.

### Example 175C: N-[(2S)-4-{[(2R,4R)-6-chloro-4-hydroxy-3,4-dihydro-2H-1-benzopyran-2-carbonyl]amino}-2-hydroxybicyclo[2.2.2]octan-1-yl]-2-ethyl-1,3-oxazole-5-carboxamide

The reaction and purification conditions described in Example 108F substituting the product of Example 175B for the product of Example 108E gave the title compound. ¹H NMR (400 MHz, DMSO-d₆) δ ppm 6.64 (s, 1H), 6.48 (dd, J=2.6, 1.0 Hz, 1H), 6.21 (ddd, J=8.7, 2.6, 0.5 Hz, 1H), 5.95 (d, J=8.7 Hz, 1H), 3.98-3.92 (m, 1H), 3.61 (dd, J=11.4, 2.4 Hz, 1H), 3.45 (ddd, J=9.5, 3.2, 1.1 Hz, 1H), 1.92 (q, J=7.6 Hz, 2H), 1.65-1.57 (m, 1H), 1.54 (ddd, J=13.1, 6.0, 2.5 Hz, 1H), 1.32-1.16 (m, 4H), 1.13-0.89 (m, 6H), 0.41 (t, J=7.6 Hz, 3H); MS (APCI⁺) m/z 490 (M+H)⁺.

### Example 176: (2R)-6-chloro-N-{3-[2-(3,4-difluorophenoxy)acetamido]bicyclo[1.1.1]pentan-1-yl}-4-oxo-3,4-dihydro-2H-1-benzopyran-2-carboxamide (Compound 275)

The title compound was prepared using the methodologies described above. ¹H NMR (400 MHz, DMSO-d₆) δ ppm 8.94 (s, 1H), 8.69 (s, 1H), 7.68-7.60 (m, 2H), 7.36 (dt, J=10.6, 9.3 Hz, 1H), 7.21-7.13 (m, 1H), 7.08 (ddd, J=12.6, 6.7, 3.0 Hz, 1H), 6.84-6.75 (m, 1H), 5.09 (t, J=7.1 Hz, 1H), 4.44 (s, 2H), 2.95 (d, J=7.1 Hz, 2H), 2.26 (s, 6H); MS (APCI⁺) m/z 477 (M+H)⁺.

### Example 177: (2S)-6-chloro-N-{3-[2-(3,4-difluorophenoxy)acetamido]bicyclo[1.1.1]pentan-1-yl}-4-oxo-3,4-dihydro-2H-1-benzopyran-2-carboxamide (Compound 276)

The title compound was prepared using the methodologies described above. ¹H NMR (400 MHz, DMSO-d₆) δ ppm 8.94 (s, 1H), 8.69 (s, 1H), 7.68-7.60 (m, 2H), 7.36 (dt, J=10.7, 9.3 Hz, 1H), 7.21-7.13 (m, 1H), 7.08 (ddd, J=12.6, 6.7, 3.0 Hz, 1H), 6.84-6.75 (m, 1H), 5.09 (t, J=7.1 Hz, 1H), 4.44 (s, 2H), 2.95 (d, J=7.1 Hz, 2H), 2.26 (s, 6H); MS (APCI⁺) m/z 477 (M+H)⁺.

### Example 178: (2R,4R)-6-chloro-N-{3-[2-(3,4-difluorophenoxy)acetamido]bicyclo[1.1.1]pentan-1-yl}-4-hydroxy-3,4-dihydro-2H-1-benzopyran-2-carboxamide (Compound 277)

The reaction and purification conditions described in Example 108F substituting the product of Example 176 for the product of Example 108E gave the title compound. ¹H NMR (400 MHz, DMSO-d₆) δ ppm 8.70 (s, 1H), 8.68 (s, 1H), 7.43-7.29 (m, 2H), 7.20 (dd, J=8.7, 2.7 Hz, 1H), 7.09 (ddd, J=12.7, 6.8, 3.1 Hz, 1H), 6.88 (d, J=8.7 Hz, 1H), 6.85-6.76 (m, 1H), 5.70 (br s, 1H), 4.80 (dd, J=10.7, 5.9 Hz, 1H), 4.59 (dd, J=12.0, 2.2 Hz, 1H), 4.44 (s, 2H), 2.35 (ddd, J=12.9, 5.9, 2.4 Hz, 1H), 2.28 (s, 6H), 1.69 (td, J=12.4, 10.8 Hz, 1H); MS (APCI⁺) m/z 461 (M−H₂O+H)⁺.

### Example 179: (2S,4S)-6-chloro-N-{3-[2-(3,4-difluorophenoxy)acetamido]bicyclo[1.1.1]pentan-1-yl}-4-hydroxy-3,4-dihydro-2H-1-benzopyran-2-carboxamide (Compound 278)

The reaction and purification conditions described in Example 108F substituting the product of Example 177 for the product of Example 108E gave the title compound. ¹H NMR (400 MHz, DMSO-d₆) δ ppm 8.69 (s, 1H), 8.67 (s, 1H), 7.43-7.29 (m, 2H), 7.20 (dd, J=8.7, 2.7 Hz, 1H), 7.09 (ddd, J=12.5, 6.7, 3.0 Hz, 1H), 6.88 (d, J=8.7 Hz, 1H), 6.84-6.76 (m, 1H), 5.69 (br s, 1H), 4.80 (dd, J=10.7, 5.9 Hz, 1H), 4.59 (dd, J=12.0, 2.3 Hz, 1H), 4.44 (s, 2H), 2.35 (ddd, J=13.0, 5.9, 2.4 Hz, 1H), 2.28 (s, 6H), 1.69 (td, J=12.6, 10.8 Hz, 1H); MS (APCI⁺) m/z 461 (M−H₂O+H)⁺.

### Example 180: (2S)-6-chloro-N-{3-[2-(4-chloro-3-fluorophenoxy)acetamido]bicyclo[1.1.1]pentan-1-yl}-2H-1-benzopyran-2-carboxamide (Compound 279)

The methodologies described in Example 208 substituting Example 96 for Example 97 gave the title compound, as well as Example 96 and Example 99. ¹H NMR (500 MHz, DMSO-d₆) δ ppm 8.71 (s, 1H), 8.69 (s, 1H), 7.49 (t, J=8.9 Hz, 1H), 7.16 (dd, J=4.5, 2.2 Hz, 2H), 7.07 (dd, J=11.3, 2.9 Hz, 1H), 6.84 (d, J=9.3 Hz, 2H), 6.53 (d, J=9.7 Hz, 1H), 5.90 (dd, J=9.9, 3.6 Hz, 1H), 5.32-5.27 (m, 1H), 4.47 (s, 2H), 2.25 (s, 6H); MS (APCI⁺) m/z 477 (M+H)⁺.

### Example 181: (2R,4R)-6-chloro-N-{3-[2-(4-chloro-3-fluorophenoxy)acetamido]bicyclo[1.1.1]pentan-1-yl}-4-hydroxy-4-methyl-3,4-dihydro-2H-1-benzopyran-2-carboxamide (Compound 280)

The methodologies described in Example 182 gave the title compound (0.134 g, 0.264 mmol, 13% yield) (as the third eluting isomer). The stereochemistry of this title compound was arbitrarily assigned (This compound is the enantiomer of Examples 138 and diastereomer of Examples 182 and 189). ¹H NMR (400 MHz, DMSO-d₆) δ ppm 8.72 (s, 2H), 7.50 (t, J=8.8 Hz, 1H), 7.44 (d, J=2.7 Hz, 1H), 7.18 (dd, J=8.7, 2.7 Hz, 1H), 7.08 (dd, J=11.3, 2.8 Hz, 1H), 6.90-6.82 (m, 2H), 5.39 (s, 1H), 4.57 (dd, J=12.6, 2.4 Hz, 1H), 4.48 (s, 2H), 2.29 (s, 6H), 2.11 (dd, J=13.4, 2.5 Hz, 1H), 1.94 (t, J=12.9 Hz, 1H), 1.45 (s, 3H); MS (APCI⁺) m/z 491 (M−H₂O+H)⁺.

### Example 182: (2R,4S)-6-chloro-N-{3-[2-(4-chloro-3-fluorophenoxy)acetamido]bicyclo[1.1.1]pentan-1-yl}-4-hydroxy-4-methyl-3,4-dihydro-2H-1-benzopyran-2-carboxamide (Compound 281)

To a stirred solution of the product of Example 87 (1.0 g, 2.0 mmol) in tetrahydrofuran (36 mL) was added methyllithium (5.1 mL, 8.11 mmol, 1.6 molar in tetrahydrofuran) at −78° C. The reaction mixture stirred at this temperature for 1 hour before being quenched with water (10 mL) and warming to ambient temperature. The resulting mixture was extracted with CH₂Cl₂ (50 mL×3). The combined organic fractions were dried over MgSO₄, filtered, and concentrated. The residue was purified by preparative HPLC (Phenomenex® Luna® C18(2) 10 μm 100 Å AXIA™ column (250 mm×50 mm). A 30-100% gradient of acetonitrile (A) and 0.1% trifluoroacetic acid in water (B) is used over 25 minutes, at a flow rate of 50 mL/minute) to give the racemic title compound as well as its stereoisomers, Examples 181 and 189. The mixture of isomers was further purified by chiral SFC (supercritical fluid chromatography) using a Daicel CHIRALPAK® AD-H column eluting with 100% CH₃OH in CO₂ with a flow rate of 48 g/minute and back pressure of 100 bar to give the title compound (fourth isomer eluted out of the column, 0.1 g, 0.2 mmol, 10% yield). The stereochemistry of this title compound was arbitrarily assigned (This compound is the enantiomer of Examples 189 and diastereomer of Examples 138 and 181). ¹H NMR (400 MHz, DMSO-d₆) δ ppm 8.71 (s, 2H), 7.50 (t, J=8.9 Hz, 1H), 7.45 (d, J=2.7 Hz, 1H), 7.27-7.16 (m, 1H), 7.08 (dd, J=11.4, 2.8 Hz, 1H), 6.97-6.80 (m, 2H), 5.42 (s, 1H), 4.55 (dd, J=12.0, 2.4 Hz, 1H), 4.48 (s, 2H), 2.28 (s, 6H), 2.10 (dd, J=13.8, 2.5 Hz, 1H), 1.83 (dd, J=13.8, 12.0 Hz, 1H), 1.48 (s, 3H); MS (APCI⁺) m/z 491 (M−H₂O+H)⁺.

### Example 183: 6-chloro-N-{3-[2-(4-chloro-3-fluorophenoxy)acetamido]bicyclo[1.1.1]pentan-1-yl}-8-methyl-4-oxo-4H-1-benzopyran-2-carboxamide (Compound 282)

### Example 183A: ethyl 6-chloro-8-methyl-4-oxo-4H-chromene-2-carboxylate

The methodologies described in Example 131A substituting 1-(5-chloro-2-hydroxy-3-methylphenyl)ethanone for 1-(2-hydroxy-4-(trifluoromethyl)phenyl)ethanone gave the title compound. ¹H NMR (400 MHz, DMSO-d₆) δ ppm 7.86 (dt, J=1.9, 0.9 Hz, 1H), 7.81 (d, J=2.5 Hz, 1H), 6.97 (s, 1H), 4.41 (q, J=7.1 Hz, 2H), 2.49 (d, J=0.8 Hz, 3H), 1.36 (t, J=7.1 Hz, 3H); MS (ESI⁺) m/z 267 (M+H)⁺.

### Example 183B: 6-chloro-8-methyl-4-oxo-4H-chromene-2-carboxylic Acid

The methodologies described in Example 131B substituting Example 183A for Example 131A gave the title compound. ¹H NMR (400 MHz, DMSO-d₆) δ ppm 7.85 (dd, J=2.6, 1.0 Hz, 1H), 7.81 (d, J=2.6 Hz, 1H), 6.93 (s, 1H), 2.49 (d, J=0.8 Hz, 3H); MS (ESI⁺) m/z 239 (M+H)⁺.

### Example 183C: 6-chloro-N-{3-[2-(4-chloro-3-fluorophenoxy)acetamido]bicyclo[1.1.1]pentan-1-yl}-8-methyl-4-oxo-4H-1-benzopyran-2-carboxamide

The methodologies described in Example 131C substituting Example 183B for Example 131B gave the title compound. ¹H NMR (400 MHz, DMSO-d₆) δ ppm 7.81 (s, 2H), 7.50 (t, J=8.8 Hz, 1H), 7.07 (dd, J=11.3, 2.8 Hz, 1H), 6.92-6.84 (m, 2H), 4.50 (s, 2H), 2.41 (s, 6H), 2.06 (s, 3H); MS (ESI⁺) m/z 505 (M+H)⁺.

### Example 184: N-{3-[2-(4-chloro-3-fluorophenoxy)acetamido]bicyclo[1.1.1]pentan-1-yl}-7-methoxy-4-oxo-4H-1-benzopyran-2-carboxamide (Compound 283)

### Example 184A: ethyl 7-methoxy-4-oxo-4H-chromene-2-carboxylate

The methodologies described in Example 131A substituting 1-(2-hydroxy-4-methoxyphenyl)ethanone for 1-(2-hydroxy-4-(trifluoromethyl)phenyl)ethanone gave the title compound. ¹H NMR (400 MHz, DMSO-d₆) δ ppm 7.96 (d, J=8.9 Hz, 1H), 7.25 (d, J=2.3 Hz, 1H), 7.12 (dd, J=8.9, 2.4 Hz, 1H), 6.90 (s, 1H), 4.40 (q, J=7.1 Hz, 2H), 3.93 (s, 3H), 1.35 (t, J=7.1 Hz, 3H); MS (ESI⁺) m/z 249 (M+H)⁺.

### Example 184B: 7-methoxy-4-oxo-4H-chromene-2-carboxylic acid

The methodologies described in Example 131B substituting Example 183A for Example 131A gave the title compound. ¹H NMR (400 MHz, DMSO-d₆) δ ppm 7.95 (d, J=8.9 Hz, 1H), 7.22 (d, J=2.4 Hz, 1H), 7.10 (dd, J=8.9, 2.4 Hz, 1H), 6.85 (s, 1H), 3.92 (s, 3H); MS (ESI⁻) m/z 221 (M+H)⁺.

### Example 184C: N-{3-[2-(4-chloro-3-fluorophenoxy)acetamido]bicyclo[1.1.1]pentan-1-yl}-7-methoxy-4-oxo-4H-1-benzopyran-2-carboxamide

The methodologies described in Example 131C substituting Example 184B for Example 131B gave the title compound. ¹H NMR (400 MHz, DMSO-d₆) δ ppm 7.97 (d, J=8.8 Hz, 1H), 7.50 (t, J=8.8 Hz, 1H), 7.21 (d, J=2.3 Hz, 1H), 7.14 (dd, J=8.9, 2.4 Hz, 1H), 7.08 (dd, J=11.3, 2.8 Hz, 1H), 6.92-6.84 (m, 1H), 6.77 (d, J=1.2 Hz, 1H), 4.50 (s, 2H), 3.93 (s, 3H), 2.41 (s, 6H); MS (ESI⁺) m/z 487 (M+H)⁺.

### Example 185: N-{3-[2-(4-chloro-3-fluorophenoxy)acetamido]bicyclo[1.1.1]pentan-1-yl}-7-methyl-4-oxo-4H-1-benzopyran-2-carboxamide (Compound 284)

### Example 185A: ethyl 7-methyl-4-oxo-4H-chromene-2-carboxylate

The methodologies described in Example 131A substituting 1-(2-hydroxy-4-methylphenyl)ethanone for 1-(2-hydroxy-4-(trifluoromethyl)phenyl)ethanone gave the title compound. ¹H NMR (400 MHz, DMSO-d₆) δ ppm 7.93 (d, J=8.1 Hz, 1H), 7.56 (t, J=1.3 Hz, 1H), 7.36 (dd, J=8.1, 1.5 Hz, 1H), 6.91 (s, 1H), 4.40 (q, J=7.1 Hz, 2H), 2.48 (s, 3H), 1.36 (t, J=7.1 Hz, 3H); MS (ESI⁺) m/z 233 (M+H)⁺.

### Example 185B: 7-methyl-4-oxo-4H-chromene-2-carboxylic Acid

The methodologies described in Example 131B substituting Example 185A for Example 131A gave the title compound. ¹H NMR (400 MHz, DMSO-d₆) δ ppm 7.90 (d, J=8.1 Hz, 1H), 7.51 (t, J=1.2 Hz, 1H), 7.32 (dd, J=8.0, 1.5 Hz, 1H), 6.84 (s, 1H), 2.44 (s, 3H); MS (ESI⁺) m/z 205 (M+H)⁺.

### Example 185C: N-{3-[2-(4-chloro-3-fluorophenoxy)acetamido]bicyclo[1.1.1]pentan-1-yl}-7-methyl-4-oxo-4H-1-benzopyran-2-carboxamide

The methodologies described in Example 131C substituting Example 185B for Example 131B gave the title compound. ¹H NMR (400 MHz, DMSO-d₆) δ ppm 9.79 (s, 1H), 8.88 (s, 1H), 7.94 (d, J=8.1 Hz, 1H), 7.58 (s, 1H), 7.50 (t, J=8.8 Hz, 1H), 7.39 (d, J=8.2 Hz, 1H), 7.08 (dd, J=11.3, 2.8 Hz, 1H), 6.92-6.84 (m, 1H), 6.80 (s, 1H), 4.50 (s, 2H), 2.49 (s, 3H), 2.40 (s, 6H); MS (ESI⁺) m/z 471 (M+H)⁺.

### Example 186: N-{3-[2-(4-chloro-3-fluorophenoxy)acetamido]bicyclo[1.1.1]pentan-1-yl}-6,7-dimethyl-4-oxo-4H-1-benzopyran-2-carboxamide (Compound 285)

### Example 186A: ethyl 6,7-dimethyl-4-oxo-4H-chromene-2-carboxylate

The methodologies described in Example 131A substituting 1-(2-hydroxy-4,5-dimethylphenyl)ethanone for 1-(2-hydroxy-4-(trifluoromethyl)phenyl)ethanone gave the title compound. ¹H NMR (400 MHz, DMSO-d₆) δ ppm 7.79 (d, J=1.0 Hz, 1H), 7.57 (s, 1H), 6.89 (s, 1H), 4.39 (q, J=7.1 Hz, 2H), 2.39 (s, 3H), 2.34 (s, 3H), 1.39-1.21 (m, 3H); MS (ESI⁺) m/z 247 (M+H)⁺.

### Example 186B: 6,7-dimethyl-4-oxo-4H-chromene-2-carboxylic Acid

The methodologies described in Example 131B substituting Example 186A for Example 131A gave the title compound. ¹H NMR (400 MHz, DMSO-d₆) δ ppm 7.79 (d, J=1.0 Hz, 1H), 7.54 (s, 1H), 6.85 (s, 1H), 2.39 (s, 3H), 2.34 (s, 3H); MS (ESI⁺) m/z 219 (M+H)⁺.

### Example 186C: N-{3-[2-(4-chloro-3-fluorophenoxy)acetamido]bicyclo[1.1.1]pentan-1-yl}-6,7-dimethyl-4-oxo-4H-1-benzopyran-2-carboxamide

The methodologies described in Example 131C substituting Example 186B for Example 131B gave the title compound. ¹H NMR (400 MHz, DMSO-d₆) δ ppm 8.88 (s, 1H), 7.79 (s, 1H), 7.56 (s, 1H), 7.50 (t, J=8.8 Hz, 1H), 7.07 (dd, J=11.3, 2.9 Hz, 1H), 6.88 (dd, J=8.7, 2.8 Hz, 1H), 6.78 (s, 1H), 4.50 (s, 2H), 2.52 (d, J=1.9 Hz, 3H), 2.40 (s, 6H), 2.35 (s, 3H).

### Example 187: N-{3-[2-(4-chloro-3-fluorophenoxy)acetamido]bicyclo[1.1.1]pentan-1-yl}-7-(difluoromethoxy)-4-oxo-4H-1-benzopyran-2-carboxamide (Compound 286)

### Example 187A: ethyl 7-(difluoromethoxy)-4-oxo-4H-chromene-2-carboxylate

The methodologies described in Example 131A substituting 1-(4-(difluoromethoxy)-2-hydroxyphenyl)ethanone for 1-(2-hydroxy-4-(trifluoromethyl)phenyl)ethanone gave the title compound. ¹H NMR (400 MHz, DMSO-d₆) δ ppm 8.10 (d, J=8.8 Hz, 1H), 7.73 (s, 1H), 7.62-7.51 (m, 1H), 7.41-7.31 (m, 1H), 6.96 (s, 1H), 4.41 (q, J=7.1 Hz, 2H), 1.36 (t, J=7.1 Hz, 3H); MS (ESI⁺) m/z 285 (M+H)⁺.

### Example 187B: 7-(difluoromethoxy)-4-oxo-4H-chromene-2-carboxylic acid

The methodologies described in Example 131B substituting Example 187A for Example 131A gave the title compound. ¹H NMR (400 MHz, DMSO-d₆) δ ppm 8.09 (d, J=8.8 Hz, 1H), 7.71 (s, 1H), 7.58-7.51 (m, 1H), 7.38-7.29 (m, 1H), 6.87 (s, 1H); MS (ESI⁻) m/z 255 (M−H)⁻.

### Example 187C: N-{3-[2-(4-chloro-3-fluorophenoxy)acetamido]bicyclo[1.1.1]pentan-1-yl}-7-(difluoromethoxy)-4-oxo-4H-1-benzopyran-2-carboxamide

The methodologies described in Example 131C substituting Example 187B for Example 131B gave the title compound. ¹H NMR (400 MHz, DMSO-d₆) δ ppm 8.11 (d, J=8.8 Hz, 1H), 7.66-7.40 (m, 3H), 7.35 (dd, J=8.8, 2.4 Hz, 1H), 7.07 (dd, J=11.3, 2.8 Hz, 1H), 6.88 (dd, J=9.0, 2.8 Hz, 1H), 6.83 (s, 1H), 4.51 (s, 2H), 2.41 (s, 6H), 2.07 (d, J=1.1 Hz, 2H); MS (ESI⁺) m/z 523 (M+H)⁺.

### Example 188: N-{3-[2-(4-chloro-3-fluorophenoxy)acetamido]bicyclo[1.1.1]pentan-1-yl}-6-methoxy-4-oxo-4H-1-benzopyran-2-carboxamide (Compound 287)

### Example 188A: ethyl 6-methoxy-4-oxo-4H-chromene-2-carboxylate

The methodologies described in Example 131A substituting 1-(2-hydroxy-5-methoxyphenyl)ethanone for 1-(2-hydroxy-4-(trifluoromethyl)phenyl)ethanone gave the title compound. MS (ESI⁺) m/z 249 (M+H)⁺.

### Example 188B: 6-methoxy-4-oxo-4H-chromene-2-carboxylic Acid

The methodologies described in Example 131B substituting Example 188A for Example 131A gave the title compound. MS (ESI⁺) m/z 221 (M+H)⁺.

### Example 188C: N-{3-[2-(4-chloro-3-fluorophenoxy)acetamido]bicyclo[1.1.1]pentan-1-yl}-6-methoxy-4-oxo-4H-1-benzopyran-2-carboxamide

The methodologies described in Example 131C substituting Example 188B for Example 131B gave the title compound. ¹H NMR (400 MHz, DMSO-d₆) δ ppm 7.73 (d, J=9.2 Hz, 1H), 7.55-7.46 (m, 2H), 7.42 (d, J=3.1 Hz, 1H), 7.08 (dd, J=11.3, 2.8 Hz, 1H), 6.88 (dd, J=9.3, 2.7 Hz, 1H), 6.82 (s, 1H), 4.50 (s, 2H), 3.87 (s, 3H), 2.40 (s, 6H); MS (APCI⁺) m/z 487 (M+H)⁺.

### Example 189: (2S,4R)-6-chloro-N-{3-[2-(4-chloro-3-fluorophenoxy)acetamido]bicyclo[1.1.1]pentan-1-yl}-4-hydroxy-4-methyl-3,4-dihydro-2H-1-benzopyran-2-carboxamide (Compound 288)

The methodologies described in Example 182 gave the title compound (0.0571 g, 0.112 mmol, 6% yield) (as the second eluting isomer). The stereochemistry of this title compound was arbitrarily assigned (This compound is the enantiomer of Examples 182 and diastereomer of Examples 138 and 181). ¹H NMR (400 MHz, DMSO-d₆) δ ppm 8.71 (s, 2H), 7.50 (t, J=8.9 Hz, 1H), 7.45 (d, J=2.7 Hz, 1H), 7.23 (dd, J=8.8, 2.6 Hz, 1H), 7.08 (dd, J=11.4, 2.8 Hz, 1H), 6.93 (d, J=8.7 Hz, 1H), 6.86 (ddd, J=8.9, 2.8, 1.2 Hz, 1H), 5.41 (s, 1H), 4.55 (dd, J=12.0, 2.5 Hz, 1H), 4.48 (s, 2H), 2.28 (s, 6H), 2.10 (dd, J=13.9, 2.5 Hz, 1H), 1.83 (dd, J=13.9, 12.0 Hz, 1H), 1.48 (s, 3H); MS (APCI⁺) m/z 491 (M−H₂O+H)⁺.

### Example 190: 6,8-dichloro-N-{3-[2-(4-chloro-3-fluorophenoxy)acetamido]bicyclo[1.1.1]pentan-1-yl}-4-oxo-4H-1-benzopyran-2-carboxamide (Compound 289)

### Example 190A: ethyl 6,8-dichloro-4-oxo-4H-chromene-2-carboxylate

The methodologies described in Example 131A substituting 1-(3,5-dichloro-2-hydroxyphenyl)ethanone for 1-(2-hydroxy-4-(trifluoromethyl)phenyl)ethanone gave the title compound. ¹H NMR (400 MHz, DMSO-d₆) δ ppm 8.27 (d, J=2.6 Hz, 1H), 7.95 (d, J=2.5 Hz, 1H), 7.04 (s, 1H), 4.41 (q, J=7.1 Hz, 2H), 1.36 (t, J=7.1 Hz, 3H); MS (ESI⁺) m/z 287 (M+H)⁺.

### Example 190B: 6,8-dichloro-4-oxo-4H-chromene-2-carboxylic Acid

The methodologies described in Example 131B substituting Example 190A for Example 131A gave the title compound. ¹H NMR (400 MHz, DMSO-d₆) δ ppm 8.26 (d, J=2.5 Hz, 1H), 7.95 (d, J=2.6 Hz, 1H), 6.98 (s, 1H); MS (ESI⁻) m/z 257 (M−H)⁻.

### Example 190C: 6,8-dichloro-N-{3-[2-(4-chloro-3-fluorophenoxy)acetamido]bicyclo[1.1.1]pentan-1-yl}-4-oxo-4H-1-benzopyran-2-carboxamide

The methodologies described in Example 131C substituting Example 190B for Example 131B gave the title compound. ¹H NMR (400 MHz, DMSO-d₆) δ ppm 8.21 (d, J=2.5 Hz, 1H), 7.95 (d, J=2.6 Hz, 1H), 7.50 (t, J=8.8 Hz, 1H), 7.07 (dd, J=11.3, 2.8 Hz, 1H), 6.93 (s, 1H), 6.88 (dd, J=9.1, 2.8 Hz, 1H), 4.50 (s, 2H), 2.39 (s, 6H).

### Example 191: N-{3-[2-(4-chloro-3-fluorophenoxy)acetamido]bicyclo[1.1.1]pentan-1-yl}-4-oxo-6-(propan-2-yl)-4H-1-benzopyran-2-carboxamide (Compound 290)

### Example 191A: ethyl 6-isopropyl-4-oxo-4H-chromene-2-carboxylate

The methodologies described in Example 131A substituting 1-(2-hydroxy-5-isopropylphenyl)ethanone for 1-(2-hydroxy-4-(trifluoromethyl)phenyl)ethanone gave the title compound. ¹H NMR (400 MHz, DMSO-d₆) δ ppm 7.87 (d, J=2.4 Hz, 1H), 7.79 (dd, J=8.8, 2.4 Hz, 1H), 7.67 (d, J=8.7 Hz, 1H), 6.93 (s, 1H), 4.41 (q, J=7.1 Hz, 2H), 3.07 (hept, J=6.9 Hz, 1H), 1.37 (t, J=7.1 Hz, 3H), 1.29 (d, J=7.1 Hz, 3H), 1.26 (d, J=5.2 Hz, 3H); MS (ESI⁺) m/z 261 (M+H)⁺.

### Example 191B: 6-isopropyl-4-oxo-4H-chromene-2-carboxylic Acid

The methodologies described in Example 131B substituting Example 191A for Example 131A gave the title compound. ¹H NMR (400 MHz, DMSO-d₆) δ ppm 7.88 (d, J=2.4 Hz, 1H), 7.79 (dd, J=8.7, 2.4 Hz, 1H), 7.67 (d, J=8.7 Hz, 1H), 6.89 (s, 1H), 3.06 (h, J=6.9 Hz, 1H), 1.26 (s, 3H), 1.25 (s, 3H); MS (ESI⁻) m/z 231 (M−H)⁻.

### Example 191C: N-{3-[2-(4-chloro-3-fluorophenoxy)acetamido]bicyclo[1.1.1]pentan-1-yl}-4-oxo-6-(propan-2-yl)-4H-1-benzopyran-2-carboxamide

The methodologies described in Example 131C substituting Example 191B for Example 131B gave the title compound. ¹H NMR (400 MHz, DMSO-d₆) δ ppm 7.88 (d, J=2.2 Hz, 1H), 7.82 (dd, J=8.7, 2.3 Hz, 1H), 7.71 (d, J=8.6 Hz, 1H), 7.50 (t, J=8.8 Hz, 1H), 7.08 (dd, J=11.2, 2.8 Hz, 1H), 6.92-6.85 (m, 1H), 6.82 (d, J=1.0 Hz, 1H), 4.50 (s, 2H), 3.07 (hept, J=7.0 Hz, 1H), 2.41 (s, 6H), 1.27 (s, 3H), 1.25 (s, 3H); MS (ESI⁺) m/z 499 (M+H)⁺.

### Example 192: N-{3-[2-(4-chloro-3-fluorophenoxy)acetamido]bicyclo[1.1.1]pentan-1-yl}-6-oxo-2H,6H-[1,3]dioxolo[4,5-h][1]benzopyran-8-carboxamide (Compound 291)

### Example 192A: ethyl 6-oxo-6H-[1,3]dioxolo[4,5-h]chromene-8-carboxylate

The methodologies described in Example 131A substituting 1-(4-hydroxybenzo[d][1,3]dioxol-5-yl)ethanone for 1-(2-hydroxy-4-(trifluoromethyl)phenyl)ethanone gave the title compound. ¹H NMR (400 MHz, DMSO-d₆) δ ppm 7.63 (d, J=8.5 Hz, 1H), 7.21 (d, J=8.4 Hz, 1H), 6.86 (s, 1H), 6.33 (s, 2H), 4.39 (q, J=7.1 Hz, 2H), 1.34 (t, J=7.1 Hz, 3H); MS (ESI⁺) m/z 263 (M+H)⁺.

### Example 192B: 6-oxo-6H-[1,3]dioxolo[4,5-h]chromene-8-carboxylic Acid

The methodologies described in Example 131B substituting Example 192A for Example 131A gave the title compound. ¹H NMR (400 MHz, DMSO-d₆) δ ppm 7.62 (d, J=8.4 Hz, 1H), 7.19 (d, J=8.5 Hz, 1H), 6.81 (s, 1H), 6.33 (s, 2H); MS (ESI⁻) m/z 233 (M−H)⁻.

### Example 192C: N-{3-[2-(4-chloro-3-fluorophenoxy)acetamido]bicyclo[1.1.1]pentan-1-yl}-6-oxo-2H,6H-[1,3]dioxolo[4,5-h][1]benzopyran-8-carboxamide

The methodologies described in Example 131C substituting Example 192B for Example 131B gave the title compound. ¹H NMR (400 MHz, DMSO-d₆) δ ppm 7.65 (dd, J=8.5, 1.2 Hz, 1H), 7.50 (t, J=8.8 Hz, 1H), 7.20 (d, J=8.5 Hz, 1H), 7.07 (dd, J=11.3, 2.9 Hz, 1H), 6.88 (dd, J=9.2, 2.8 Hz, 1H), 6.75 (d, J=1.1 Hz, 1H), 6.33 (s, 1H), 4.50 (s, 1H), 2.53 (t, J=2.6 Hz, 6H), 2.38 (s, 2H); MS (ESI⁻⁺) m/z 168 (M+3H)⁺.

### Example 193: N-{3-[2-(4-chloro-3-fluorophenoxy)acetamido]bicyclo[1.1.1]pentan-1-yl}-6,8-difluoro-4-oxo-4H-1-benzopyran-2-carboxamide (Compound 292)

### Example 193A: ethyl 6,8-difluoro-4-oxo-4H-chromene-2-carboxylate

The methodologies described in Example 131A substituting 1-(3,5-difluoro-2-hydroxyphenyl)ethanone for 1-(2-hydroxy-4-(trifluoromethyl)phenyl)ethanone gave the title compound. ¹H NMR (400 MHz, DMSO-d₆) δ ppm 8.04 (ddd, J=10.9, 8.6, 3.0 Hz, 1H), 7.60 (ddd, J=8.1, 3.0, 1.8 Hz, 1H), 7.02 (s, 1H), 4.42 (q, J=7.1 Hz, 2H), 1.36 (t, J=7.1 Hz, 3H); MS (ESI⁺) m/z 255 (M+H)⁺.

### Example 193B: 6,8-difluoro-4-oxo-4H-chromene-2-carboxylic acid

The methodologies described in Example 131B substituting Example 193A for Example 131A gave the title compound. ¹H NMR (400 MHz, DMSO-d₆) δ ppm 8.02 (ddd, J=10.9, 8.6, 3.0 Hz, 1H), 7.60 (ddd, J=8.2, 3.0, 1.8 Hz, 1H), 6.96 (s, 1H); MS (ESI⁻) m/z 225 (M−H)⁻.

### Example 193C: N-{3-[2-(4-chloro-3-fluorophenoxy)acetamido]bicyclo[1.1.1]pentan-1-yl}-6,8-difluoro-4-oxo-4H-1-benzopyran-2-carboxamide

The methodologies described in Example 131C substituting Example 193B for Example 131B gave the title compound. ¹H NMR (400 MHz, DMSO-d₆) δ ppm 7.94 (ddd, J=11.0, 8.7, 3.0 Hz, 1H), 7.59 (ddd, J=8.2, 3.1, 1.6 Hz, 1H), 7.50 (t, J=8.9 Hz, 1H), 7.07 (dd, J=11.3, 2.8 Hz, 1H), 6.92-6.84 (m, 2H), 4.50 (s, 2H), 2.40 (s, 6H).

### Example 194: N-(4-{[rac-(2R,4R)-6-chloro-4-hydroxy-3,4-dihydro-2H-1-benzopyran-2-carbonyl]amino}bicyclo[2.1.1]hexan-1-yl)-5-(trifluoromethoxy)pyridine-2-carboxamide (Compound 293)

The reaction and purification conditions described in Example 108F substituting the product of Example 212 for the product of Example 108E gave the title compound. ¹H NMR (501 MHz, DMSO-d₆) δ ppm 9.09 (s, 1H), 8.73-8.68 (m, 1H), 8.47 (s, 1H), 8.15 (dd, J=8.7, 0.7 Hz, 1H), 8.11-8.05 (m, 1H), 7.39 (dd, J=2.8, 1.0 Hz, 1H), 7.20 (ddd, J=8.8, 2.8, 0.8 Hz, 1H), 6.90 (d, J=8.7 Hz, 1H), 5.71 (s, 1H), 4.81 (dd, J=10.8, 5.9 Hz, 1H), 4.61 (dd, J=11.9, 2.2 Hz, 1H), 2.35 (ddd, J=12.9, 5.9, 2.3 Hz, 1H), 2.17-2.13 (m, 2H), 1.99-1.94 (m, 2H), 1.94-1.83 (m, 4H), 1.79-1.68 (m, 1H); MS (ESI⁻) m/z 510 (M−H)⁻.

### Example 195: 8-chloro-N-{3-[2-(4-chloro-3-fluorophenoxy)acetamido]bicyclo[1.1.1]pentan-1-yl}-4-oxo-4H-1-benzopyran-2-carboxamide (Compound 294)

### Example 195A: ethyl 8-chloro-4-oxo-4H-chromene-2-carboxylate

The methodologies described in Example 131A substituting 1-(3-chloro-2-hydroxyphenyl)ethanone for 1-(2-hydroxy-4-(trifluoromethyl)phenyl)ethanone gave the title compound. ¹H NMR (400 MHz, DMSO-d₆) δ ppm 8.11-7.89 (m, 1H), 7.54 (t, J=7.9 Hz, 1H), 7.15-6.99 (m, 1H), 4.46-4.15 (m, 2H), 1.40-1.21 (m, 3H); MS (ESI⁺) m/z 253 (M+H)⁺.

### Example 195B: 8-chloro-4-oxo-4H-chromene-2-carboxylic Acid

The methodologies described in Example 131B substituting Example 195A for Example 131A gave the title compound. ¹H NMR (400 MHz, DMSO-d₆) δ ppm 8.09-7.97 (m, 2H), 7.92 (d, J=2.1 Hz, 0.4H), 7.78 (dd, J=8.5, 2.2 Hz, 0.4H), 7.53 (t, J=7.9 Hz, 1H), 7.08 (dd, J=19.9, 8.6 Hz, 1H), 6.96 (s, 1H); MS (ESI⁺) m/z 225 (M+H)⁺.

### Example 195C: 8-chloro-N-{3-[2-(4-chloro-3-fluorophenoxy)acetamido]bicyclo[1.1.1]pentan-1-yl}-4-oxo-4H-1-benzopyran-2-carboxamide

The methodologies described in Example 131C substituting Example 195B for Example 131B gave the title compound. ¹H NMR (400 MHz, DMSO-d₆) δ ppm 8.07-7.97 (m, 2H), 7.52 (dt, J=19.3, 8.3 Hz, 2H), 7.07 (dd, J=11.3, 2.8 Hz, 1H), 6.91 (s, 1H), 6.88 (dd, J=9.1, 2.8 Hz, 1H), 4.50 (s, 2H), 2.40 (s, 6H).

### Example 196: N-{3-[2-(4-chloro-3-fluorophenoxy)acetamido]bicyclo[1.1.1]pentan-1-yl}-7-hydroxy-4-oxo-4H-1-benzopyran-2-carboxamide (Compound 295)

### Example 196A: 7-hydroxy-4-oxo-4H-chromene-2-carboxylic Acid

The methodologies described in Example 131B substituting ethyl 7-hydroxy-4-oxo-4H-chromene-2-carboxylate (Guo B, et al. Bioorg. Med. Chem., 2018, 26, 5780-5791) for Example 131A gave the title compound. ¹H NMR (400 MHz, DMSO-d₆) δ ppm 10.98 (s, 1H), 7.89 (d, J=8.8 Hz, 1H), 6.96 (dd, J=8.8, 2.2 Hz, 1H), 6.91 (d, J=2.2 Hz, 1H), 6.79 (s, 1H); MS (ESI⁺) m/z 207 (M+H)⁺.

### Example 196B: N-{3-[2-(4-chloro-3-fluorophenoxy)acetamido]bicyclo[1.1.1]pentan-1-yl}-7-hydroxy-4-oxo-4H-1-benzopyran-2-carboxamide

The methodologies described in Example 131C substituting Example 196A for Example 131B gave the title compound. ¹H NMR (400 MHz, DMSO-d₆) δ ppm 8.88 (s, 1H), 7.91 (d, J=8.7 Hz, 1H), 7.50 (t, J=8.9 Hz, 1H), 7.12-6.95 (m, 3H), 6.88 (dd, J=8.9, 2.8 Hz, 1H), 6.73 (d, J=1.3 Hz, 1H), 4.50 (s, 2H), 2.40 (s, 6H); MS (ESI⁺) m/z 473 (M+H)⁺.

### Example 197: rac-(2R,4R)-6-chloro-N-{4-[2-(4-chloro-3-fluorophenoxy)acetamido]bicyclo[2.1.1]hexan-1-yl}-4-hydroxy-3,4-dihydro-2H-1-benzopyran-2-carboxamide (Compound 296)

The reaction and purification conditions described in Example 108F substituting the product of Example 211 for the product of Example 108E gave the title compound. ¹H NMR (501 MHz, DMSO-d₆) δ ppm 8.49 (s, 1H), 8.41 (s, 1H), 7.49 (t, J=8.9 Hz, 1H), 7.38 (dd, J=2.7, 1.0 Hz, 1H), 7.21-7.17 (m, 1H), 7.07 (dd, J=11.4, 2.9 Hz, 1H), 6.89 (d, J=8.7 Hz, 1H), 6.85 (ddd, J=9.0, 2.9, 1.2 Hz, 1H), 5.69 (d, J=5.2 Hz, 1H), 4.85-4.75 (m, 1H), 4.59 (dd, J=11.9, 2.2 Hz, 1H), 4.48 (s, 2H), 2.34 (ddd, J=12.9, 6.0, 2.3 Hz, 1H), 2.12-2.05 (m, 2H), 1.86-1.79 (m, 6H), 1.77-1.67 (m, 1H); MS (ESI⁻) m/z 507 (M−H)⁻.

### Example 198: 6-chloro-N-{3-[2-(4-chloro-3-fluorophenoxy)acetamido]bicyclo[1.1.1]pentan-1-yl}-4-ethyl-4-hydroxy-3,4-dihydro-2H-1-benzopyran-2-carboxamide (Compound 297)

To a stirred solution of the product of Example 87 (0.071 g, 0.144 mmol) in tetrahydrofuran (2.6 mL) was added ethyllithium (1.2 mL, 0.58 mmol, 0.5 molar in benzene/cyclohexane) at −78° C. The reaction mixture stirred at this temperature for 2 hours before being quenched with water (10 mL) and warming to ambient temperature. The resulting mixture was extracted with CH₂Cl₂ (10 mL×3). The combined organic fractions were dried over MgSO₄, filtered, and concentrated. The residue was purified by preparative HPLC (Phenomenex® Luna® C18(2) 10 μm 100 Å AXIA™ column (250 mm×50 mm). A 30-100% gradient of acetonitrile (A) and 0.1% trifluoroacetic acid in water (B) is used over 25 minutes, at a flow rate of 50 mL/minute) to give the title compound (0.033 g, 0.063 mmol, 44% yield). ¹H NMR (400 MHz, DMSO-d₆, dr 1.5:1) δ ppm 8.94 (s, 1H), 8.72 (d, J=2.6 Hz, 4H), 7.66-7.62 (m, 1H), 7.49 (td, J=8.9, 2.1 Hz, 2H), 7.38 (dd, J=5.7, 2.7 Hz, 2H), 7.26-7.16 (m, 1H), 7.20-7.13 (m, 1H), 7.07 (dt, J=11.4, 2.8 Hz, 3H), 6.98-6.84 (m, 3H), 6.85 (qd, J=3.0, 1.2 Hz, 2H), 5.29 (d, J=45.9 Hz, 2H), 5.09 (t, J=7.1 Hz, 1H), 4.56 (dd, J=12.8, 2.7 Hz, 2H), 4.54-4.49 (m, 1H), 4.48 (s, 4H), 4.47 (s, 2H), 2.95 (d, J=7.1 Hz, 2H), 2.28 (s, 9H), 2.26 (s, 6H), 1.83-1.60 (m, 4H), 0.90 (t, J=7.3 Hz, 4H), 0.75 (t, J=7.3 Hz, 1H); MS (APCI⁺) m/z 505 (M−H₂O+H)⁺.

### Example 199: N-{3-[2-(4-chloro-3-fluorophenoxy)acetamido]bicyclo[1.1.1]pentan-1-yl}-6-fluoro-4-(hydroxyimino)-3,4-dihydro-2H-1-benzopyran-2-carboxamide (Compound 298)

The methodologies described in Example 122 substituting 6-fluoro-4-(hydroxyimino)chroman-2-carboxylic acid for 6,7-dimethoxy-4-oxo-4H-chromene-2-carboxylic acid gave the title compound. ¹H NMR (501 MHz, DMSO-d₆) δ ppm 11.57 (s, 1H), 8.80 (s, 1H), 8.74 (s, 1H), 7.49 (t, J=8.9 Hz, 1H), 7.45 (dd, J=9.4, 3.1 Hz, 1H), 7.17 (ddd, J=8.8, 8.1, 3.1 Hz, 1H), 7.11-7.03 (m, 2H), 6.86 (ddd, J=9.0, 2.9, 1.1 Hz, 1H), 4.57 (dd, J=10.8, 4.0 Hz, 1H), 4.50 (s, 2H), 3.27 (dd, J=17.3, 4.0 Hz, 1H), 2.63 (dd, J=17.3, 10.8 Hz, 1H), 2.30 (s, 6H); MS (APCI⁺) m/z 492 (M+H)⁺.

### Example 200: (2S,4S)-6-chloro-4-hydroxy-N-[3-(2-{[6-(trifluoromethyl)pyridin-3-yl]oxy}acetamido)bicyclo[1.1.1]pentan-1-yl]-3,4-dihydro-2H-1-benzopyran-2-carboxamide (Compound 299)

The reaction and purification conditions described in Example 108F substituting the product of Example 236 for the product of Example 108E gave the title compound. ¹H NMR (500 MHz, DMSO-d₆) δ ppm 8.82 (s, 1H), 8.69 (s, 1H), 8.47 (d, J=2.9 Hz, 1H), 7.87 (dd, J=8.8, 0.6 Hz, 1H), 7.60-7.54 (m, 1H), 7.38 (dd, J=2.7, 1.0 Hz, 1H), 7.20 (ddd, J=8.7, 2.7, 0.7 Hz, 1H), 6.88 (d, J=8.7 Hz, 1H), 5.70 (d, J=4.6 Hz, 1H), 4.83-4.77 (m, 1H), 4.67 (s, 2H), 4.60 (dd, J=12.0, 2.2 Hz, 1H), 2.38-2.31 (m, 1H), 1.74-1.64 (m, 1H); MS (ESI⁻) m/z 510 (M−H)⁻.

### Example 201: N-{3-[2-(4-chloro-3-fluorophenoxy)acetamido]bicyclo[1.1.1]pentan-1-yl}-6-fluoro-4-hydroxy-3,4-dihydro-2H-1-benzopyran-2-carboxamide (Compound 300)

The methodologies described in Example 91 substituting Example 233 for Example 87 and purifying by preparative HPLC [Waters XBridge™ C18 5 μm OBD column, 30×100 mm, flow rate 40 mL/minute, 5-100% gradient of acetonitrile in buffer (0.025 M aqueous ammonium bicarbonate, adjusted to pH 10 with ammonium hydroxide)] gave the title compound as the major product (59% yield) and Example 231 as a minor product (4% yield). ¹H NMR (400 MHz, DMSO-d₆) δ ppm 8.72 (s, 1H), 8.65 (s, 1H), 7.50 (t, J=8.9 Hz, 1H), 7.14 (dd, J=9.4, 3.1 Hz, 1H), 7.08 (dd, J=11.4, 2.8 Hz, 1H), 7.00 (td, J=8.6, 3.2 Hz, 1H), 6.94-6.82 (m, 2H), 5.67 (d, J=6.0 Hz, 1H), 4.80 (dt, J=11.2, 5.7 Hz, 1H), 4.56 (dd, J=12.1, 2.2 Hz, 1H), 4.48 (s, 2H), 2.40-2.26 (m, 1H), 2.28 (s, 6H), 1.69 (td, J=12.4, 10.7 Hz, 1H); MS (APCI⁺) m/z 461 (M−H₂O+H)+.

### Example 202: rac-(2R,4S)-6-chloro-N-{3-[2-(4-chloro-3-fluorophenoxy)acetamido]bicyclo[1.1.1]pentan-1-yl}-4-[(2-hydroxyethyl)amino]-3,4-dihydro-2H-1-benzopyran-2-carboxamide (Compound 301)

Example 89 was purified by chiral SFC (supercritical fluid chromatography) using a Regis® Technologies, Inc. Whelk-O®1 column eluting with 40% CH₃OH with 0.1% diethylamine in CO₂ with a flow rate of 80 g/minute and back pressure of 120 bar to give the title compound (second isomer eluted out of the column, 0.027 g, 0.050 mmol, 35% yield). The stereochemistry of this title compound was arbitrarily assigned (This compound is the stereoisomer of Examples 209 and 210.). ¹H NMR (400 MHz, DMSO-d₆) δ ppm 8.71 (s, 1H), 8.69 (s, 1H), 7.50 (t, J=8.9 Hz, 1H), 7.30 (d, J=2.6 Hz, 1H), 7.22 (dd, J=8.8, 2.7 Hz, 1H), 7.08 (dd, J=11.4, 2.8 Hz, 1H), 6.92 (d, J=8.7 Hz, 1H), 6.85 (ddd, J=9.0, 2.9, 1.3 Hz, 1H), 4.63 (dd, J=11.1, 2.6 Hz, 1H), 4.52 (t, J=5.5 Hz, 1H), 4.48 (s, 2H), 3.73-3.67 (m, 1H), 3.54-3.43 (m, 2H), 2.72 (dt, J=11.6, 5.8 Hz, 1H), 2.64 (dt, J=11.7, 5.9 Hz, 1H), 2.28 (s, 6H), 2.16 (dt, J=13.9, 3.0 Hz, 1H), 1.80-1.68 (m, 1H); MS (APCI⁺) m/z 538 (M+H)⁺.

### Example 203: N-(4-{[rac-(2R,4R)-6-chloro-4-hydroxy-3,4-dihydro-2H-1-benzopyran-2-carbonyl]amino}bicyclo[2.2.2]octan-1-yl)-5-(difluoromethyl)pyrazine-2-carboxamide (Compound 302)

The reaction and purification conditions described in Example 108F substituting the product of Example 205 for the product of Example 108E gave the title compound. ¹H NMR (501 MHz, DMSO-d₆) δ ppm 9.22 (d, J=1.4 Hz, 1H), 8.98 (d, J=1.2 Hz, 1H), 8.02 (s, 1H), 7.73 (s, 1H), 7.65-7.59 (m, 2H), 7.20 (t, J=54.0 Hz, 1H), 7.16 (dd, J=8.7, 0.5 Hz, 1H), 5.06 (dd, J=8.3, 5.0 Hz, 1H), 2.99-2.87 (m, 2H), 2.07-1.88 (m, 12H); MS (ESI⁺) m/z 505 (M+H)⁺.

### Example 204: 6-chloro-N-{(3S)-4-[2-(4-chloro-3-fluorophenoxy)acetamido]-3-hydroxybicyclo[2.2.2]octan-1-yl}-4-(methylamino)-3,4-dihydro-2H-1-benzopyran-2-carboxamide (Compound 303)

The methodologies described in Example 89 substituting methanamine hydrochloride for 2-((trimethylsilyl)oxy)ethanamine, substituting Example 100C for Example 87, and purifying using preparative HPLC [Waters XBridge™ C18 5 μm OBD column, 30×100 mm, flow rate 40 mL/minute, 5-100% gradient of acetonitrile in buffer (0.025 M aqueous ammonium bicarbonate, adjusted to pH 10 with ammonium hydroxide)] gave the title compound. ¹H NMR (400 MHz, DMSO-d₆) δ ppm 7.53-7.44 (m, 2H), 7.30 (dd, J=16.4, 14.4 Hz, 2H), 7.23-7.12 (m, 1H), 7.06 (dd, J=11.4, 2.8 Hz, 1H), 6.88 (dd, J=10.2, 8.7 Hz, 1H), 6.83 (ddd, J=9.0, 2.9, 1.1 Hz, 1H), 5.08 (dt, J=4.4, 1.3 Hz, 1H), 4.54 (ddd, J=36.6, 10.8, 2.6 Hz, 1H), 4.47 (s, 2H), 4.08-4.02 (m, 1H), 3.83 (dd, J=10.5, 5.4 Hz, 1H), 3.53 (t, J=3.9 Hz, 1H), 2.32 (s, 1H), 2.31-2.27 (m, 1H), 2.27 (s, 3H), 2.07 (s, 1H), 1.94 (d, J=12.7 Hz, 1H), 1.88 (s, 1H), 1.82 (s, 5H), 1.80 (d, J=5.2 Hz, 1H), 1.80-1.61 (m, 1H); MS (APCI⁺) m/z 556 (M+H)⁺.

### Example 205: N-{4-[(6-chloro-4-oxo-3,4-dihydro-2H-1-benzopyran-2-carbonyl)amino]bicyclo[2.2.2]octan-1-yl}-5-(difluoromethyl)pyrazine-2-carboxamide (Compound 304)

### Example 205A: N-(4-aminobicyclo[2.2.2]octan-1-yl)-5-(difluoromethyl)pyrazine-2-carboxamide, 3 trifluoroacetic acid

The reaction and purification conditions described in Example 108E substituting the product of Example 1A for the product of Example 108A, and 5-(difluoromethyl)pyrazine-2-carboxylic acid (PharmaBlock) for the product of Example 108D gave the tert-butoxycarbonyl protected intermediate [tert-butyl (4-(5-(difluoromethyl)pyrazine-2-carboxamido)bicyclo[2.2.2]octan-1-yl)carbamate] which was further processed with trifluoroacetic acid as described in Example 114C to give the title compound. MS (APCI⁺) m/z 297 (M+H)⁺.

### Example 205B: N-{4-[(6-chloro-4-oxo-3,4-dihydro-2H-1-benzopyran-2-carbonyl)amino]bicyclo[2.2.2]octan-1-yl}-5-(difluoromethyl)pyrazine-2-carboxamide

The reaction and purification conditions described in Example 108E substituting the product of Example 205A for the product of Example 108A, and 6-chloro-4-oxochroman-2-carboxylic acid (Princeton) for the product of Example 108D gave the title compound. ¹H NMR (501 MHz, DMSO-d₆) δ ppm 9.22 (d, J=1.4 Hz, 1H), 8.98 (d, J=1.2 Hz, 1H), 8.02 (s, 1H), 7.73 (s, 1H), 7.65-7.59 (m, 2H), 7.20 (t, J=54.0 Hz, 1H), 7.16 (dd, J=8.7, 0.5 Hz, 1H), 5.06 (dd, J=8.3, 5.0 Hz, 1H), 2.99-2.87 (m, 2H), 2.07-1.88 (m, 12H); MS (ESI⁺) m/z 505 (M+H)⁺.

### Example 206: 4-amino-6-chloro-N-{3-[2-(4-chloro-3-fluorophenoxy)acetamido]bicyclo[1.1.1]pentan-1-yl}-3,4-dihydro-2H-1-benzopyran-2-carboxamide (Compound 305)

To a solution of the product of Example 87 (0.200 g, 0.405 mmol) in acetonitrile (2.7 mL) was added ammonium acetate (0.125 g, 1.62 mmol) and zinc chloride (powdered, 0.166 g, 1.22 mmol). After stirring at 50° C. for 5 minutes, sodium cyanoborohydride (0.076 g, 1.22 mmol) was added, and this mixture was allowed to stir at 60° C. for 8 hours. Then the reaction mixture was cooled to ambient temperature, diluted with a drop of water, and concentrated. The residue was diluted with N,N-dimethylformamide/water (1.2 mL, 3:1) and purified by preparative HPLC (Waters XBridge™ C18 5 μm OBD column, 30×100 mm, flow rate 40 mL/minute, 5-100% gradient of acetonitrile in 0.1% trifluoroacetic acid/water) to give the title compound (0.10 g, 0.20 mmol, 50% yield) as a mixture of diastereomers (dr 1:1). ¹H NMR (501 MHz, DMSO-d₆, dr 1:1) δ ppm 8.88 (s, 1H), 8.84 (s, 1H), 8.75 (s, 1H), 8.74 (s, 1H), 8.54 (s, 2H), 8.47 (s, 2H), 7.60 (d, J=2.5 Hz, 1H), 7.55 (d, J=2.7 Hz, 1H), 7.50 (td, J=8.9, 1.3 Hz, 2H), 7.38 (ddd, J=17.3, 8.8, 2.6 Hz, 2H), 7.23 (s, 1H), 7.13 (s, 1H), 7.07 (dt, J=11.4, 2.3 Hz, 2H), 7.06-6.98 (m, 3H), 6.86 (ddt, J=9.0, 2.9, 1.4 Hz, 2H), 4.70 (dd, J=10.7, 3.0 Hz, 1H), 4.65 (dd, J=11.7, 2.2 Hz, 1H), 4.49 (s, 2H), 4.48 (s, 2H), 2.56 (ddd, J=13.0, 6.0, 2.2 Hz, 1H), 2.39-2.32 (m, 1H), 2.30 (s, 6H), 2.28 (s, 6H), 2.19 (ddd, J=15.2, 10.7, 5.2 Hz, 1H), 1.85 (dt, J=13.3, 11.6 Hz, 1H); MS (APCI⁺) m/z 494 (M+H)⁺.

### Example 207: (2S,4S)-6-chloro-N-{4-[2-(4-chloro-3-fluorophenoxy)acetamido]bicyclo[2.2.2]octan-1-yl}-4-hydroxy-3,4-dihydro-2H-1-benzopyran-2-carboxamide (Compound 306)

Example 238 was purified by chiral SFC (supercritical fluid chromatography) using a Daicel CHIRALPAK® AD-H column eluting with 100% CH₃OH in CO₂ with a flow rate of 80 g/minute and back pressure of 120 bar to give the title compound (second enantiomer eluted out of the column, 0.0170 g, 0.032 mmol, 41% yield). The stereochemistry of this title compound was arbitrarily assigned (This compound is the enantiomer of Example 130.). ¹H NMR (500 MHz, DMSO-d₆) δ ppm 7.52-7.44 (m, 2H), 7.37 (dd, J=2.7, 1.0 Hz, 1H), 7.31 (s, 1H), 7.17 (dd, J=8.7, 2.6 Hz, 1H), 7.03 (dd, J=11.5, 2.9 Hz, 1H), 6.86 (d, J=8.7 Hz, 1H), 6.81 (ddd, J=8.9, 2.9, 1.2 Hz, 1H), 5.70 (s, 1H), 4.77 (dd, J=10.7, 6.0 Hz, 1H), 4.55 (dd, J=11.8, 2.3 Hz, 1H), 4.44 (s, 2H), 2.26 (ddd, J=13.0, 6.0, 2.3 Hz, 1H), 1.92 (s, 12H), 1.72 (dt, J=12.7, 11.0 Hz, 1H); MS (APCI⁺) m/z 520 (M−H₂O+H)⁺.

### Example 208: (2R)-6-chloro-N-{3-[2-(4-chloro-3-fluorophenoxy)acetamido]bicyclo[1.1.1]pentan-1-yl}-2H-1-benzopyran-2-carboxamide (Compound 307)

A solution of the product of Example 97 (0.553 g, 1.12 mmol) in trifluoroacetic acid (8.5 mL, 111 mmol) was stirred at 35° C. for 1 hour. Then the solution was concentrated in vacuo. The residue was taken up in acetonitrile (5 mL) and then NH₄OH (5 mL, 5% aqueous) was added. After stirring at ambient temperature for 10 minutes, the mixture was concentrated in vacuo. The residue was purified by preparative HPLC [YMC TriArt™ C18 Hybrid 5 m column, 50×100 mm, flow rate 70 mL/minute, 5-100% gradient of acetonitrile in buffer (0.025 M aqueous ammonium bicarbonate, adjusted to pH 10 with ammonium hydroxide)] to give the title compound (0.016 g, 0.034 mmol, 3% yield) as well as Example 97 and Example 98. ¹H NMR (400 MHz, DMSO-d₆) δ ppm 8.71 (s, 1H), 8.69 (s, 1H), 7.49 (t, J=8.9 Hz, 1H), 7.20-7.13 (m, 2H), 7.07 (dd, J=11.4, 2.8 Hz, 1H), 6.89-6.81 (m, 2H), 6.53 (dd, J=9.9, 2.1 Hz, 1H), 5.91 (dd, J=9.9, 3.6 Hz, 1H), 5.30 (dd, J=3.6, 2.2 Hz, 1H), 4.48 (s, 2H), 2.26 (s, 6H); MS (APCI⁺) m/z 477 (M+H)⁺.

### Example 209: (2R,4R)-6-chloro-N-{3-[2-(4-chloro-3-fluorophenoxy)acetamido]bicyclo[1.1.1]pentan-1-yl}-4-[(2-hydroxyethyl)amino]-3,4-dihydro-2H-1-benzopyran-2-carboxamide (Compound 308)

Example 89 was purified by chiral SFC (supercritical fluid chromatography) using a Whelk-O®1 column eluting with 40% CH₃OH with 0.1% diethylamine in CO₂ with a flow rate of 80 g/minute and back pressure of 120 bar to give the title compound (third isomer eluted out of the column, 0.014 g, 0.026 mmol, 18% yield). The stereochemistry of this title compound was arbitrarily assigned (This compound is the diastereomer of Examples 202 and enantiomer of Example 210.). ¹H NMR (400 MHz, DMSO-d₆) δ ppm 8.72 (s, 1H), 8.63 (s, 1H), 7.55-7.45 (m, 2H), 7.18 (dd, J=8.7, 2.7 Hz, 1H), 7.08 (dd, J=11.4, 2.9 Hz, 1H), 6.87 (dd, J=10.6, 7.6 Hz, 2H), 4.54 (dd, J=10.9, 2.6 Hz, 1H), 4.48 (s, 2H), 3.94 (dd, J=10.3, 5.3 Hz, 1H), 3.47 (t, J=5.9 Hz, 2H), 2.67 (dt, J=11.9, 6.0 Hz, 1H), 2.59-2.51 (m, 1H), 2.38 (ddd, J=13.1, 5.4, 2.7 Hz, 1H), 2.28 (s, 6H), 1.68 (dt, J=13.1, 10.6 Hz, 1H), 1.02 (t, J=6.9 Hz, 2H); MS (APCI⁺) m/z 538 (M+H)⁺.

### Example 210: (2S,4S)-6-chloro-N-{3-[2-(4-chloro-3-fluorophenoxy)acetamido]bicyclo[1.1.1]pentan-1-yl}-4-[(2-hydroxyethyl)amino]-3,4-dihydro-2H-1-benzopyran-2-carboxamide (Compound 309)

Example 89 was purified by chiral SFC (supercritical fluid chromatography) using a Whelk-O®1 column eluting with 40% CH₃OH with 0.1% diethylamine in CO₂ with a flow rate of 80 g/minute and back pressure of 120 bar to give the title compound (first isomer eluted out of the column, 0.015 g, 0.028 mmol, 20% yield). The stereochemistry of this title compound was arbitrarily assigned (This compound is the diastereomer of Examples 202 and enantiomer of Example 209.). ¹H NMR (400 MHz, DMSO-d₆) δ ppm 8.71 (s, 1H), 8.62 (s, 1H), 7.55-7.45 (m, 2H), 7.18 (dd, J=8.5, 2.7 Hz, 1H), 7.08 (dd, J=11.4, 2.8 Hz, 1H), 6.92-6.82 (m, 2H), 4.54 (dd, J=10.9, 2.6 Hz, 1H), 4.48 (s, 3H), 3.94 (dd, J=10.3, 5.3 Hz, 1H), 3.47 (q, J=5.8 Hz, 2H), 2.67 (dt, J=11.8, 6.1 Hz, 1H), 2.56 (q, J=5.7 Hz, 1H), 2.38 (ddd, J=13.0, 5.3, 2.7 Hz, 1H), 2.28 (s, 6H), 2.14-2.08 (m, 1H), 1.74-1.61 (m, 1H); MS (APCI⁺) m/z 538 (M+H)⁺.

### Example 211: 6-chloro-N-{4-[2-(4-chloro-3-fluorophenoxy)acetamido]bicyclo[2.1.1]hexan-1-yl}-4-oxo-3,4-dihydro-2H-1-benzopyran-2-carboxamide (Compound 310)

### Example 211A: N-(4-aminobicyclo[2.1.1]hexan-1-yl)-6-chloro-4-oxochroman-2-carboxamide, trifluoroacetic Acid

Trifluoroacetic acid (5 mL) was added to the product of Example 213 (0.40 g, 0.88 mmol) and the resulting solution was stirred at 55° C. for 2.5 hours. The reaction mixture was cooled to ambient temperature and concentrated under reduced pressure to give the title compound (0.34 g, 0.78 mmol, 89% yield). ¹H NMR (400 MHz, methanol-d₄) δ ppm 7.77 (d, J=2.6 Hz, 1H), 7.55 (dd, J=8.9, 2.7 Hz, 1H), 7.15 (d, J=8.9 Hz, 1H), 5.04 (dd, J=8.6, 6.1 Hz, 1H), 3.03-2.92 (m, 2H), 2.22-2.13 (m, 2H), 2.07-1.86 (m, 6H); MS (ESI⁺) m/z 312 (M+H)⁺.

### Example 211B: 6-chloro-N-{4-[2-(4-chloro-3-fluorophenoxy)acetamido]bicyclo[2.1.1]hexan-1-yl}-4-oxo-3,4-dihydro-2H-1-benzopyran-2-carboxamide

The reaction and purification conditions described in Example 108E substituting the product of Example 211A for the product of Example 108A, and 2-(4-chloro-3-fluorophenoxy)acetic acid for the product of Example 108D gave the title compound. ¹H NMR (501 MHz, DMSO-d₆) δ ppm 8.69 (s, 1H), 8.47 (s, 1H), 7.66-7.61 (m, 2H), 7.49 (t, J=8.9 Hz, 1H), 7.17 (dd, J=8.7, 0.6 Hz, 1H), 7.06 (dd, J=11.4, 2.8 Hz, 1H), 6.85 (ddd, J=9.0, 2.9, 1.2 Hz, 1H), 5.09 (t, J=7.0 Hz, 1H), 4.47 (s, 2H), 2.95 (d, J=7.0 Hz, 2H), 2.08-2.02 (m, 2H), 1.83-1.74 (m, 6H); MS (ESI⁻) m/z 505 (M−H)⁻.

### Example 212: N-{4-[(6-chloro-4-oxo-3,4-dihydro-2H-1-benzopyran-2-carbonyl)amino]bicyclo[2.1.1]hexan-1-yl}-5-(trifluoromethoxy)pyridine-2-carboxamide (Compound 311)

The reaction and purification conditions described in Example 108E substituting 5-(trifluoromethoxy)picolinic acid (Enamine) for the product of Example 108D, and the product of Example 211A for the product of Example 108A gave the title compound. ¹H NMR (501 MHz, DMSO-d₆) δ ppm 9.06 (s, 1H), 8.72 (s, 1H), 8.70-8.68 (m, 1H), 8.13 (dd, J=8.7, 0.7 Hz, 1H), 8.09-8.04 (m, 1H), 7.67-7.60 (m, 2H), 7.18 (d, J=8.7 Hz, 1H), 5.10 (t, J=7.0 Hz, 1H), 2.99-2.94 (m, 2H), 2.15-2.08 (m, 2H), 1.94-1.87 (m, 4H), 1.84-1.77 (m, 2H); MS (ESI⁺) m/z 510 (M+H)⁺.

### Example 213: benzyl {4-[(6-chloro-4-oxo-3,4-dihydro-2H-1-benzopyran-2-carbonyl)amino]bicyclo[2.1.1]hexan-1-yl}carbamate (Compound 312)

The reaction and purification conditions described in Example 108E substituting benzyl (4-aminobicyclo[2.1.1]hexan-1-yl)carbamate hydrochloride (Enamine) for the product of Example 108A, and 6-chloro-4-oxochroman-2-carboxylic acid (Princeton) for the product of Example 108D gave the title compound. ¹H NMR (400 MHz, DMSO-d₆) δ ppm 8.66 (s, 1H), 7.77 (s, 1H), 7.66-7.59 (m, 2H), 7.40-7.27 (m, 5H), 7.17 (dd, J=8.6, 0.7 Hz, 1H), 5.08 (t, J=6.9 Hz, 1H), 4.99 (s, 2H), 2.95 (d, J=6.9 Hz, 2H), 2.04 (br s, 2H), 1.79-1.62 (m, 6H); MS (ESI⁺) m/z 472 (M+H)⁺.

### Example 214: N-{3-[2-(4-chloro-3-fluorophenoxy)acetamido]bicyclo[1.1.1]pentan-1-yl}-7-fluoro-4-oxo-4H-1-benzopyran-2-carboxamide (Compound 313)

The methodologies described in Example 122 substituting 7-fluoro-4-oxo-4H-chromene-2-carboxylic acid for 6,7-dimethoxy-4-oxo-4H-chromene-2-carboxylic acid gave the title compound. ¹H NMR (501 MHz, DMSO-d₆) δ ppm 9.66 (s, 1H), 8.80 (s, 1H), 8.12 (dd, J=8.9, 6.4 Hz, 1H), 7.55 (dd, J=9.3, 2.4 Hz, 1H), 7.50 (t, J=8.9 Hz, 1H), 7.43 (td, J=8.7, 2.5 Hz, 1H), 7.09 (dd, J=11.4, 2.8 Hz, 1H), 6.87 (ddd, J=9.0, 2.9, 1.2 Hz, 1H), 6.81 (s, 1H), 4.51 (s, 2H), 2.39 (s, 6H); MS (APCI⁺) m/z 475 (M+H)⁺.

### Example 215: 6-chloro-N-{3-[2-(4-chloro-3-fluorophenoxy)acetamido]bicyclo[1.1.1]pentan-1-yl}-4-(methylamino)-3,4-dihydro-2H-1-benzopyran-2-carboxamide (Compound 314)

The methodologies described in Example 89 substituting methanamine hydrochloride for 2-((trimethylsilyl)oxy)ethanamine and purifying using preparative HPLC [Waters XBridge™ C18 5 μm OBD column, 30×100 mm, flow rate 40 mL/minute, 5-100% gradient of acetonitrile in buffer (0.025 M aqueous ammonium bicarbonate, adjusted to pH 10 with ammonium hydroxide)] gave the title compound. ¹H NMR (400 MHz, DMSO-d₆, dr 1:1) δ ppm 8.72 (s, 2H), 8.70 (s, 1H), 8.65 (s, 1H), 7.54-7.45 (m, 3H), 7.30 (d, J=2.7 Hz, 1H), 7.25-7.14 (m, 2H), 7.08 (dd, J=11.4, 2.8 Hz, 2H), 6.95-6.82 (m, 4H), 4.60 (dd, J=11.1, 2.6 Hz, 1H), 4.54 (dd, J=11.3, 2.4 Hz, 1H), 4.48 (s, 4H), 3.87 (m, 1H), 3.54 (m, 1H), 2.43-2.34 (m, 1H), 2.33 (s, 3H), 2.30-2.27 (m, 12H), 2.27 (s, 3H), 2.22-2.14 (m, 1H), 2.12 (s, 2H), 1.77-1.57 (m, 2H); MS (APCI⁺) m/z 508 (M+H)⁺.

### Example 216: 6-chloro-N-{3-[2-(4-chloro-3-fluorophenoxy)acetamido]bicyclo[1.1.1]pentan-1-yl}-4-hydroxy-4H-1-benzopyran-2-carboxamide (Compound 315)

To a cooled (0° C.) solution of the product of Example 219 (0.072 g, 0.15 mmol) in tetrahydrofuran (2.4 mL) was added borane dimethyl sulfide complex (0.15 mL, 0.29 mmol). This reaction mixture was allowed to stir at 0° C. for 2.5 hours. Although the conversion was incomplete, the reaction mixture was quenched with methanol (1 mL), and concentrated. The residue was diluted with N,N-dimethylformamide/water (3 mL), and purified by preparative HPLC [Waters XBridge™ C18 5 μm OBD column, 30×100 mm, flow rate 40 mL/minute, 5-100% gradient of acetonitrile in buffer (0.025 M aqueous ammonium bicarbonate, adjusted to pH 10 with ammonium hydroxide)] to give the title compound (0.015 g, 0.030 mmol, 21% yield) as the major product in an impure mixture. ¹H NMR (501 MHz, DMSO-d₆, dr 2.5:1) δ ppm 8.77 (s, 2H), 8.73 (s, 2H), 8.71 (s, 1H), 8.69 (s, 1H), 8.00-7.93 (m, 2H), 7.78 (d, J=8.9 Hz, 1H), 7.50 (td, J=8.9, 4.7 Hz, 4H), 7.27 (dd, J=8.6, 2.6 Hz, 2H), 7.23 (d, J=2.6 Hz, 3H), 7.19-7.14 (m, 2H), 7.08 (ddd, J=8.6, 6.1, 3.2 Hz, 6H), 6.85 (dt, J=9.1, 3.2 Hz, 8H), 6.53 (d, J=11.3 Hz, 1H), 5.95 (t, J=4.0 Hz, 2H), 5.90 (dd, J=9.8, 3.7 Hz, 1H), 4.49 (s, 5H), 4.47 (s, 2H), 2.31 (s, 15H), 2.26 (d, J=9.6 Hz, 6H); MS (APCI⁺) m/z 477 (M−H₂O+H)⁺.

### Example 217: (2S,4R)-6-chloro-N-{3-[2-(4-chloro-3-fluorophenoxy)acetamido]bicyclo[1.1.1]pentan-1-yl}-4-{[(1s,3S)-3-hydroxycyclobutyl]amino}-3,4-dihydro-2H-1-benzopyran-2-carboxamide (Compound 316)

Example 110 was purified by chiral SFC (supercritical fluid chromatography) using a Regis Technologies, Inc. Whelk-O®1 column eluting with 40% CH₃OH with 0.1% diethylamine in CO₂ with a flow rate of 80 g/minute and back pressure of 100 bar to give the title compound (second isomer eluted out of the column, 0.009 g, 0.016 mmol, 39% yield). The stereochemistry of this title compound was arbitrarily assigned (This compound is the stereoisomer of Example 229.). ¹H NMR (400 MHz, DMSO-d₆) δ ppm 8.71 (s, 1H), 8.68 (s, 1H), 7.50 (t, J=8.9 Hz, 1H), 7.27-7.17 (m, 2H), 7.07 (dd, J=11.4, 2.8 Hz, 1H), 6.91 (d, J=8.7 Hz, 1H), 6.85 (ddd, J=9.0, 2.9, 1.2 Hz, 1H), 4.91 (d, J=5.7 Hz, 1H), 4.63 (dd, J=11.1, 2.5 Hz, 1H), 4.48 (s, 2H), 3.78 (h, J=7.2 Hz, 1H), 3.63 (s, 1H), 2.93 (q, J=7.3 Hz, 1H), 2.79 (s, 1H), 2.47 (t, J=5.4 Hz, 1H), 2.27 (s, 6H), 2.11 (d, J=13.7 Hz, 1H), 1.66 (ddd, J=33.8, 24.9, 10.6 Hz, 3H), 1.16 (t, J=7.3 Hz, 1H); MS (APCI⁺) m/z 564 (M+H)⁺.

### Example 218: 7-chloro-N-{3-[2-(4-chloro-3-fluorophenoxy)acetamido]bicyclo[1.1.1]pentan-1-yl}-2H-1-benzopyran-3-carboxamide (Compound 317)

The methodologies described in Example 122 substituting 7-chloro-2H-chromene-3-carboxylic acid for 6,7-dimethoxy-4-oxo-4H-chromene-2-carboxylic acid gave the title compound. ¹H NMR (501 MHz, DMSO-d₆) δ ppm 8.82 (s, 1H), 8.73 (s, 1H), 7.50 (t, J=8.9 Hz, 1H), 7.26-7.19 (m, 2H), 7.08 (dd, J=11.4, 2.8 Hz, 1H), 7.01 (dd, J=8.1, 2.0 Hz, 1H), 6.94 (dd, J=2.0, 0.7 Hz, 1H), 6.86 (ddd, J=8.9, 2.8, 1.1 Hz, 1H), 4.91 (d, J=1.4 Hz, 2H), 4.48 (s, 2H), 2.29 (s, 6H); MS (APCI⁺) m/z 477 (M+H)⁺.

### Example 219: 6-chloro-N-{3-[2-(4-chloro-3-fluorophenoxy)acetamido]bicyclo[1.1.1]pentan-1-yl}-4-oxo-4H-1-benzopyran-2-carboxamide (Compound 318)

The methodologies described in Example 122 substituting 6-chloro-4-oxo-4H-chromene-2-carboxylic acid for 6,7-dimethoxy-4-oxo-4H-chromene-2-carboxylic acid gave the title compound. ¹H NMR (400 MHz, DMSO-d₆) δ ppm 9.72 (s, 1H), 8.80 (s, 1H), 8.00-7.91 (m, 1H), 7.78 (d, J=8.9 Hz, 1H), 7.50 (t, J=8.9 Hz, 1H), 7.09 (dd, J=11.4, 2.9 Hz, 1H), 6.87 (ddd, J=9.0, 2.9, 1.2 Hz, 1H), 6.84 (s, 1H), 4.51 (s, 2H), 2.39 (s, 6H); MS (APCI⁺) m/z 491 (M+H)⁺.

### Example 220: (2S,4S)-6-chloro-N-{3-[2-(4-chloro-3-fluorophenoxy)acetamido]bicyclo[1.1.1]pentan-1-yl}-4-[(2,2,2-trifluoroethyl)amino]-3,4-dihydro-2H-1-benzopyran-2-carboxamide (Compound 319)

Example 237 was purified by chiral SFC (supercritical fluid chromatography) using a Regis Technologies, Inc. Whelk-O®1 column eluting with 40% CH₃OH with 0.1% diethylamine in CO₂ with a flow rate of 80 g/minute and back pressure of 100 bar to give the title compound (first isomer eluted out of the column, 0.007 g, 0.012 mmol, 60% yield). The stereochemistry of this title compound was arbitrarily assigned (This compound is the stereoisomer of Examples 224 and 228.). ¹H NMR (400 MHz, DMSO-d₆) δ ppm 8.72 (s, 1H), 8.67 (s, 1H), 7.54-7.45 (m, 2H), 7.40-7.30 (m, 1H), 7.20 (dd, J=8.7, 2.7 Hz, 1H), 7.06 (td, J=11.6, 2.8 Hz, 1H), 6.90 (d, J=8.7 Hz, 1H), 6.88-6.78 (m, 1H), 4.55 (dd, J=11.5, 2.3 Hz, 1H), 4.48 (s, 2H), 4.02 (td, J=10.0, 5.7 Hz, 1H), 3.29-3.22 (m, 1H), 2.92 (q, J=7.8 Hz, 1H), 2.43 (ddd, J=13.1, 5.5, 2.4 Hz, 1H), 2.28 (s, 6H), 1.87 (d, J=2.7 Hz, 1H), 1.65 (dt, J=13.2, 11.2 Hz, 1H); MS (APCI⁺) m/z 576 (M+H)⁺.

### Example 221: (2R)-5-chloro-N-{3-[2-(4-chloro-3-fluorophenoxy)acetamido]bicyclo[1.1.1]pentan-1-yl}-2-methyl-3-oxo-2,3-dihydro-1-benzofuran-2-carboxamide (Compound 320)

The methodologies described in Example 160 substituting Example 159 for Example 158 gave solely the title compound. ¹H NMR (400 MHz, DMSO-d₆) δ ppm 8.75 (s, 1H), 8.70 (s, 1H), 7.83 (dd, J=8.9, 2.3 Hz, 1H), 7.72 (d, J=2.3 Hz, 1H), 7.48 (t, J=8.9 Hz, 1H), 7.39 (d, J=8.9 Hz, 1H), 7.06 (dd, J=11.4, 2.8 Hz, 1H), 6.85 (ddd, J=9.0, 2.9, 1.2 Hz, 1H), 4.47 (s, 2H), 2.23 (s, 6H), 1.61 (s, 3H); MS (APCI⁺) m/z 493 (M+H)⁺.

### Example 222: (2S)—N-{3-[2-(3,4-difluorophenoxy)acetamido]bicyclo[1.1.1]pentan-1-yl}-6,7-difluoro-4-oxo-3,4-dihydro-2H-1-benzopyran-2-carboxamide (Compound 321)

The reaction and purification conditions described in Example 108E substituting the product of Example 172A for the product of Example 108A, and the product of Example 109A for the product of Example 108D gave the title compound. ¹H NMR (400 MHz, DMSO-d₆) δ ppm 8.93 (s, 1H), 8.70 (s, 1H), 7.68 (dd, J=10.4, 9.0 Hz, 1H), 7.36 (dt, J=10.7, 9.3 Hz, 1H), 7.26 (dd, J=11.4, 6.5 Hz, 1H), 7.08 (ddd, J=12.6, 6.7, 3.0 Hz, 1H), 6.79 (dtd, J=9.1, 3.3, 1.7 Hz, 1H), 5.11 (t, J=7.1 Hz, 1H), 4.44 (s, 2H), 2.98-2.92 (m, 2H), 2.26 (s, 6H); MS (APCI⁺) m/z 479 (M+H)⁺.

### Example 223: N-{3-[2-(4-chloro-3-fluorophenoxy)acetamido]bicyclo[1.1.1]pentan-1-yl}-6-methoxy-4-oxo-4H-pyrano[3,2-b]pyridine-2-carboxamide (Compound 322)

### Example 223A: 6-methoxy-4-oxo-4H-pyrano[3,2-b]pyridine-2-carboxylic Acid

The methodologies described in Example 132A substituting 1-(3-hydroxy-6-methoxypyridin-2-yl)ethanone for 1-(5-chloro-4-fluoro-2-hydroxyphenyl)ethanone gave the title compound. ¹H NMR (400 MHz, DMSO-d₆) δ ppm 8.18 (d, J=9.1 Hz, 1H), 7.35 (d, J=9.1 Hz, 1H), 7.02 (s, 1H), 3.96 (s, 3H); MS (APCI⁺) m/z 222 (M+H)⁺.

### Example 223B: N-{3-[2-(4-chloro-3-fluorophenoxy)acetamido]bicyclo[1.1.1]pentan-1-yl}-6-methoxy-4-oxo-4H-pyrano[3,2-b]pyridine-2-carboxamide

The methodologies described in Example 122 substituting Example 223A for 6,7-dimethoxy-4-oxo-4H-chromene-2-carboxylic acid gave the title compound. ¹H NMR (400 MHz, DMSO-d₆) δ ppm 9.64 (s, 1H), 8.75 (s, 1H), 8.05 (d, J=9.1 Hz, 1H), 7.47 (t, J=8.9 Hz, 1H), 7.33 (d, J=9.1 Hz, 1H), 7.05 (dd, J=11.4, 2.9 Hz, 1H), 6.87 (s, 1H), 6.83 (ddd, J=9.0, 2.9, 1.2 Hz, 1H), 4.47 (s, 2H), 3.92 (s, 3H), 2.35 (s, 6H); MS (APCI⁺) m/z 489 (M+H)⁺.

### Example 224: (2S,4R)-6-chloro-N-{3-[2-(4-chloro-3-fluorophenoxy)acetamido]bicyclo[1.1.1]pentan-1-yl}-4-[(2,2,2-trifluoroethyl)amino]-3,4-dihydro-2H-1-benzopyran-2-carboxamide (Compound 323)

Example 237 was purified by chiral SFC (supercritical fluid chromatography) using a Regis Technologies, Inc. Whelk-O®1 column eluting with 40% CH₃OH with 0.1% diethylamine in CO₂ with a flow rate of 80 g/minute and back pressure of 100 bar to give the title compound (second isomer eluted out of the column, 0.003 g, 0.0052 mmol, 26% yield). The stereochemistry of this title compound was arbitrarily assigned (This compound is the a diastereomer of Examples 220 and the enantiomer of Example 228.). ¹H NMR (400 MHz, DMSO-d₆) δ ppm 8.71 (s, 1H), 8.69 (s, 1H), 7.50 (t, J=8.9 Hz, 1H), 7.34 (d, J=2.7 Hz, 1H), 7.24 (dd, J=8.8, 2.7 Hz, 1H), 7.07 (dd, J=11.4, 2.8 Hz, 1H), 6.93 (d, J=8.8 Hz, 1H), 6.88-6.79 (m, 1H), 4.61 (dd, J=10.7, 2.7 Hz, 1H), 4.48 (s, 2H), 3.76 (m, 1H), 3.31 (m, 1H), 2.91 (q, J=7.3 Hz, 1H), 2.28 (s, 6H), 2.17 (d, J=13.9 Hz, 1H), 1.86-1.75 (m, 1H); MS (APCI⁺) m/z 576 (M+H)⁺.

### Example 225: 6-chloro-N-{3-[2-(4-chloro-3-fluorophenoxy)acetamido]bicyclo[1.1.1]pentan-1-yl}-N-methyl-4-oxo-4H-1-benzopyran-2-carboxamide (Compound 324)

### Example 225A: N-(3-(benzyl(methyl)amino)bicyclo[1.1.1]pentan-1-yl)-2-(4-chloro-3-fluorophenoxy)acetamide

To a solution of Example 23B (0.15 g, 0.53 mmol) in 2.4 weight % sodium acetate trihydrate and 3.6 weight % acetic acid in methanol (5.3 mL) was added benzaldehyde (0.06 mL, 0.55 mmol). To this cooled (0° C.) reaction mixture was added sodium cyanoborohydride (0.1 g, 1.58 mmol). The mixture warmed to ambient temperature as the ice melted over 1 hour. Then to the benzylamine intermediate in the reaction mixture was added formaldehyde (0.041 mL, 0.55 mmol) and sodium cyanoborohydride (0.1 g, 1.58 mmol). After stirring at ambient temperature for 2 hours, the reaction mixture was concentrated, and the residue was purified by preparative HPLC (Waters XBridge™ C18 5 μm OBD column, 30×100 mm, flow rate 40 mL/minute, 5-100% gradient of acetonitrile in 0.1% trifluoroacetic acid/water) to give the title intermediate. MS (APCI⁺) m/z 389 (M+H)⁺.

### Example 225B: 2-(4-chloro-3-fluorophenoxy)-N-(3-(methylamino)bicyclo[1.1.1]pentan-1-yl)acetamide

To a mixture of Example 225A (0.0529 g, 0.136 mmol) and 5% Pd/C (wet, 36.3 weight %, 12.5 mg, 0.043 mmol) in tetrahydrofuran (1.0 mL) in a 20 mL RS10 reactor with a glass liner was added 4 M HCl in dioxane (0.10 mL, 0.40 mmol). The reactor was closed, purged with argon, and the mixture was stirred at 1200 RPM under 50 psi of hydrogen at 25° C. for 100 hours. The reaction mixture was filtered, the filtrate was concentrated, and a portion of the residue was purified by preparative HPLC (Phenomenex® Luna® C18(2) 10 μm 100 Å AXIA™ column (250 mm×50 mm). A 30-100% gradient of acetonitrile (A) and 0.1% trifluoroacetic acid in water (B) is used over 25 minutes, at a flow rate of 50 mL/minute) to give the title intermediate. 7.03 (m, 1H), 6.85 (ddd, J=8.9, 2.8, 1.2 Hz, 1H), 4.51 (s, 2H), 2.55 (s, 3H), 2.25 (s, 6H); MS (APCI⁺) m/z 505 (M+H)⁺.

### Example 225C: 6-chloro-N-{3-[2-(4-chloro-3-fluorophenoxy)acetamido]bicyclo[1.1.1]pentan-1-yl}-N-methyl-4-oxo-4H-1-benzopyran-2-carboxamide

The methodologies described in Example 122 substituting Example 225B for Example 23B and substituting 6-chloro-4-oxo-4H-chromene-2-carboxylic acid for 6,7-dimethoxy-4-oxo-4H-chromene-2-carboxylic acid gave the title compound. ¹H NMR (400 MHz, DMSO-d6, 90° C. to resolve rotamers) δ ppm 8.39 (s, 1H), 7.99 (d, J=2.6 Hz, 1H), 7.86 (dd, J=9.0, 2.7 Hz, 1H), 7.75 (d, J=8.9 Hz, 1H), 7.41 (t, J=8.8 Hz, 1H), 6.97 (dd, J=11.3, 2.8 Hz, 1H), 6.80 (ddd, J=8.9, 2.8, 1.2 Hz, 1H), 6.53 (s, 1H), 4.41 (s, 2H), 3.00 (s, 3H), 2.30 (s, 6H); MS (APCI⁺) m/z 505 (M+H)⁺.

### Example 226: (2R)-6-chloro-N-{3-[2-(4-chloro-3-fluorophenoxy)acetamido]bicyclo[1.1.1]pentan-1-yl}-4-[(1-hydroxycyclopropyl)methyl]-3,4-dihydro-2H-1,4-benzoxazine-2-carboxamide (Compound 325)

### Example 226A: (R)-4-((1-((tert-butyldimethylsilyl)oxy)cyclopropyl)methyl)-6-chloro-N-(3-(2-(4-chloro-3-fluorophenoxy)acetamido)bicyclo[1.1.1]pentan-1-yl)-3,4-dihydro-2H-benzo[b][1,4]oxazine-2-carboxamide

The methodologies described in Example 88 substituting the product of Example 3 for Example 14, substituting 1-((tert-butyldimethylsilyl)oxy)cyclopropanecarbaldehyde for 2-methoxy-2-methylpropanal, and purifying by preparative HPLC (Waters XBridge™ C18 5 μm OBD column, 30×100 mm, flow rate 40 mL/minute, 5-100% gradient of acetonitrile in 0.1% trifluoroacetic acid/water) gave the title intermediate. MS (ESI⁺) m/z 664 (M+H)⁺.

### Example 226B: (2R)-6-chloro-N-{3-[2-(4-chloro-3-fluorophenoxy)acetamido]bicyclo[1.1.1]pentan-1-yl}-4-[(I-hydroxycyclopropyl)methyl]-3,4-dihydro-2H-1,4-benzoxazine-2-carboxamide

To a solution of Example 226A (0.0003 g, 0.451 μmol) in dichloromethane (3 μL) was added trifluoroacetic acid (0.1 μL, 1 μmol), and the reaction mixture was allowed to stir at ambient temperature for 1.5 hours. Then the reaction mixture was concentrated to give the title compound (0.0002 g, 0.363 μmol, 81% yield). ¹H NMR (500 MHz, DMSO-d₆) δ ppm 8.77 (s, 1H), 8.75 (s, 1H), 7.54 (t, J=8.9 Hz, 1H), 7.11 (dd, J=11.4, 2.9 Hz, 1H), 6.89 (dd, J=9.1, 2.6 Hz, 2H), 6.82 (d, J=8.4 Hz, 1H), 6.59 (dd, J=8.4, 2.4 Hz, 1H), 4.54 (dd, J=7.4, 2.8 Hz, 1H), 4.52 (s, 2H), 3.44-3.33 (m, 3H), 2.30 (s, 6H), 1.29 (d, J=9.6 Hz, 2H), 0.88 (s, 2H), 0.65-0.52 (m, 4H), 0.04 (s, 1H), 0.00 (s, 1H); MS (APCI⁺) m/z 550 (M+H)⁺.

### Example 227: 6-chloro-N-{4-[2-(4-chloro-3-fluorophenoxy)acetamido]bicyclo[2.2.2]octan-1-yl}-4-oxo-3,4-dihydro-2H-1-benzopyran-2-carboxamide (Compound 326)

### Example 227A: tert-butyl (4-(6-chloro-4-oxochroman-2-carboxamido)bicyclo[2.2.2]octan-1-yl)carbamate

The methodologies described in Example 122 substituting Example 1A for Example 23B and substituting 6-chloro-4-oxochroman-2-carboxylic acid for 6,7-dimethoxy-4-oxo-4H-chromene-2-carboxylic acid gave the title intermediate. MS (APCI*) m/z 490 (M+H)⁺.

### Example 227B: N-(4-aminobicyclo[2.2.2]octan-1-yl)-6-chloro-4-oxochroman-2-carboxamide

To a solution of Example 227A (0.158 g, 0.352 mmol) in dichloromethane (2.3 mL) was added trifluoroacetic acid (0.14 mL, 1.8 mmol), and the reaction mixture was allowed to stir at ambient temperature for 1.5 hours. Then the reaction mixture was concentrated and carried forward without purification. MS (APCI⁺) m/z 349 (M+H)⁺.

### Example 227C: 6-chloro-N-{4-[2-(4-chloro-3-fluorophenoxy)acetamido]bicyclo[2.2.2]octan-1-yl}-4-oxo-3,4-dihydro-2H-1-benzopyran-2-carboxamide

The methodologies described in Example 122 substituting Example 227B for Example 23B and substituting 2-(4-chloro-3-fluorophenoxy)acetic acid for 6,7-dimethoxy-4-oxo-4H-chromene-2-carboxylic acid gave the title compound. ¹H NMR (400 MHz, DMSO-d₆) δ ppm 7.68 (s, 1H), 7.65-7.57 (m, 2H), 7.52-7.43 (m, 2H), 7.15 (d, J=8.6 Hz, 1H), 7.02 (dd, J=11.4, 2.9 Hz, 1H), 6.80 (ddd, J=9.0, 2.9, 1.2 Hz, 1H), 5.04 (dd, J=8.2, 5.0 Hz, 1H), 4.42 (s, 2H), 3.00-2.83 (m, 2H), 1.87 (d, J=2.4 Hz, 12H); MS (APCI⁺) m/z 535 (M+H)⁺.

### Example 228: (2R,4S)-6-chloro-N-{3-[2-(4-chloro-3-fluorophenoxy)acetamido]bicyclo[1.1.1]pentan-1-yl}-4-[(2,2,2-trifluoroethyl)amino]-3,4-dihydro-2H-1-benzopyran-2-carboxamide (Compound 327)

Example 237 was purified by chiral SFC (supercritical fluid chromatography) using a Regis Technologies, Inc. Whelk-O®1 column eluting with 40% CH₃OH with 0.1% diethylamine in CO₂ with a flow rate of 80 g/minute and back pressure of 100 bar to give the title compound (third isomer eluted out of the column, 0.005 g, 0.0087 mmol, 43% yield). The stereochemistry of this title compound was arbitrarily assigned (This compound is the diastereomer of Examples 220 and enantiomer of Example 224.). ¹H NMR (400 MHz, DMSO-d₆) δ ppm 8.71 (s, 1H), 8.69 (s, 1H), 7.50 (t, J=8.8 Hz, 1H), 7.34 (d, J=2.6 Hz, 1H), 7.24 (dd, J=8.8, 2.7 Hz, 1H), 7.07 (dd, J=11.4, 2.8 Hz, 1H), 6.93 (d, J=8.7 Hz, 1H), 6.85 (ddd, J=9.0, 2.8, 1.2 Hz, 1H), 4.61 (dd, J=10.6, 2.7 Hz, 1H), 4.48 (s, 2H), 3.76 (d, J=6.1 Hz, 1H), 3.42-3.36 (m, 1H), 2.91 (q, J=7.2 Hz, 1H), 2.28 (s, 6H), 2.17 (dt, J=14.2, 3.4 Hz, 1H), 1.80 (ddd, J=14.3, 10.7, 4.1 Hz, 1H); MS (APCI⁺) m/z 576 (M+H)⁺.

### Example 229: (2R,4R)-6-chloro-N-{3-[2-(4-chloro-3-fluorophenoxy)acetamido]bicyclo[1.1.1]pentan-1-yl}-4-{[(1s,3S)-3-hydroxycyclobutyl]amino}-3,4-dihydro-2H-1-benzopyran-2-carboxamide (Compound 328)

Example 110 was purified by chiral SFC (supercritical fluid chromatography) using a Regis Technologies, Inc. Whelk-O®1 column eluting with 40% CH₃OH with 0.1% diethylamine in CO₂ with a flow rate of 80 g/minute and back pressure of 100 bar to give the title compound (first isomer eluted out of the column, 0.007 g, 0.012 mmol, 31% yield). The stereochemistry of this title compound was arbitrarily assigned (This compound is the stereoisomer of Example 217.). ¹H NMR (501 MHz, DMSO-d₆) δ ppm 8.72 (s, 1H), 8.63 (s, 1H), 7.53-7.46 (m, 2H), 7.19-7.14 (m, 1H), 7.08 (dd, J=11.4, 2.9 Hz, 1H), 6.89-6.83 (m, 2H), 4.89 (d, J=6.0 Hz, 1H), 4.51 (dd, J=11.2, 2.5 Hz, 1H), 4.48 (s, 2H), 3.86 (s, 1H), 3.73 (q, J=7.1 Hz, 1H), 2.73 (s, 1H), 2.64 (d, J=2.0 Hz, 2H), 2.48-2.39 (m, 2H), 2.38-2.29 (m, 1H), 2.28 (s, 6H), 1.66-1.54 (m, 3H); MS (APCI⁺) m/z 564 (M+H)⁺.

### Example 230: (2R)-6-chloro-4-oxo-N-[3-(2-{[2-(trifluoromethyl)pyrimidin-5-yl]oxy}acetamido)bicyclo[1.1.1]pentan-1-yl]-3,4-dihydro-2H-1-benzopyran-2-carboxamide (Compound 329)

The reaction and purification conditions described in Example 128C and Example 234 substituting 2-(trifluoromethyl)pyrimidin-5-ol for 2-methoxypyrimidin-5-ol gave the title compound. ¹H NMR (400 MHz, DMSO-d₆) δ ppm 8.95 (s, 1H), 8.85 (s, 1H), 8.75 (s, 2H), 7.68-7.60 (m, 2H), 7.21-7.13 (m, 1H), 5.09 (t, J=7.1 Hz, 1H), 4.79 (s, 2H), 2.95 (d, J=7.1 Hz, 2H), 2.26 (s, 6H); MS (ESI⁺) m/z 511 (M+H)⁺.

### Example 231: N-{3-[2-(4-chloro-3-fluorophenoxy)acetamido]bicyclo[1.1.1]pentan-1-yl}-6-fluoro-4-methoxy-3,4-dihydro-2H-1-benzopyran-2-carboxamide (Compound 330)

The methodologies described in Example 91 substituting Example 233 for Example 87 and purifying by preparative HPLC (Waters XBridge™ C18 5 μm OBD column, 30×100 mm, flow rate 40 mL/minute, 5-100% gradient of acetonitrile in 0.1% trifluoroacetic acid/water) gave the title compound as a minor product (4% yield) and Example 201 as the major product (59% yield). ¹H NMR (400 MHz, DMSO-d₆) δ ppm 8.71 (s, 1H), 8.60 (s, 1H), 7.50 (t, J=8.9 Hz, 1H), 7.11-7.00 (m, 3H), 6.96-6.82 (m, 2H), 4.62-4.53 (m, 2H), 4.48 (s, 2H), 3.38 (s, 3H), 2.27 (s, 6H), 1.81 (dt, J=13.0, 9.8 Hz, 1H); MS (APCI⁺) m/z 493 (M+H)⁺.

### Example 232: (2R)-6-chloro-N-(3-{2-[(5-chloropyridin-2-yl)oxy]acetamido}bicyclo[1.1.1]pentan-1-yl)-4-oxo-3,4-dihydro-2H-1-benzopyran-2-carboxamide (Compound 331)

The reaction and purification conditions described in Example 108E substituting 2-((5-chloropyridin-2-yl)oxy)acetic acid (Enamine) for the product of Example 108D, and the product of Example 128B for the product of Example 108A gave the title compound. ¹H NMR (500 MHz, DMSO-d₆) δ ppm 8.92 (s, 1H), 8.65 (s, 1H), 8.18 (dd, J=2.8, 0.6 Hz, 1H), 7.84 (dd, J=8.8, 2.7 Hz, 1H), 7.68-7.60 (m, 2H), 7.20-7.12 (m, 1H), 6.95 (dd, J=8.8, 0.7 Hz, 1H), 5.08 (t, J=7.1 Hz, 1H), 4.66 (s, 2H), 2.94 (d, J=7.1 Hz, 2H), 2.23 (s, 6H); MS (ESI⁺) m/z 476 (M+H)⁺.

### Example 233: N-{3-[2-(4-chloro-3-fluorophenoxy)acetamido]bicyclo[1.1.1]pentan-1-yl}-6-fluoro-4-oxo-3,4-dihydro-2H-1-benzopyran-2-carboxamide (Compound 332)

The methodologies described in Example 122 substituting 6-fluoro-4-oxochroman-2-carboxylic acid for 6,7-dimethoxy-4-oxo-4H-chromene-2-carboxylic acid gave the title compound. ¹H NMR (501 MHz, DMSO-d₆) δ ppm 8.92 (s, 1H), 8.72 (s, 1H), 7.53-7.45 (m, 2H), 7.43 (dd, J=8.4, 3.2 Hz, 1H), 7.18 (dd, J=9.1, 4.3 Hz, 1H), 7.07 (dd, J=11.4, 2.8 Hz, 1H), 6.85 (ddd, J=9.0, 2.9, 1.2 Hz, 1H), 5.06 (dd, J=9.1, 5.4 Hz, 1H), 4.48 (s, 2H), 2.97-2.87 (m, 2H), 2.26 (s, 6H); MS (APCI⁺) m/z 477 (M−H₂O+H)⁺.

### Example 234: (2R)-6-chloro-N-(3-{2-[(2-methoxypyrimidin-5-yl)oxy]acetamido}bicyclo[1.1.1]pentan-1-yl)-4-oxo-3,4-dihydro-2H-1-benzopyran-2-carboxamide (Compound 333)

The reaction and purification conditions described in Example 108E substituting the product of Example 128B for the product of Example 108A, and the product of Example 128C for the product of Example 108D gave the title compound. ¹H NMR (400 MHz, DMSO-d₆) δ ppm 8.94 (s, 1H), 8.74 (s, 1H), 8.35 (s, 2H), 7.68-7.60 (m, 2H), 7.21-7.13 (m, 1H), 5.09 (t, J=7.0 Hz, 1H), 4.54 (s, 2H), 3.86 (s, 3H), 2.95 (d, J=7.0 Hz, 2H), 2.26 (s, 6H); MS (APCI⁺) m/z 473 (M+H)⁺.

### Example 235: rac-(2R,4R)—N-{3-[2-(4-chloro-3-fluorophenoxy)acetamido]bicyclo[1.1.1]pentan-1-yl}-4-hydroxy-6-methoxy-3,4-dihydro-2H-pyrano[3,2-b]pyridine-2-carboxamide (Compound 334)

The methodologies described in Example 123 substituting Example 223 for Example 214 gave the title compound. ¹H NMR (500 MHz, DMSO-d₆, dr 25:1) δ ppm 8.73 (s, 1H), 8.65 (s, 1H), 7.50 (t, J=8.9 Hz, 1H), 7.32 (d, J=8.8 Hz, 0.04H), 7.25 (d, J=8.8 Hz, 1H), 7.08 (dd, J=11.4, 2.8 Hz, 1H), 6.86 (ddd, J=9.0, 2.9, 1.2 Hz, 1H), 6.73 (d, J=8.8 Hz, 0.04H), 6.67 (dd, J=8.8, 0.7 Hz, 1H), 4.77-4.70 (m, 1H), 4.57 (dd, J=11.0, 2.6 Hz, 1H), 4.48 (s, 2H), 3.83 (s, 3H), 2.41 (ddd, J=13.3, 6.3, 2.6 Hz, 1H), 2.28 (s, 6H), 1.89 (ddd, J=13.3, 11.0, 9.5 Hz, 1H); MS (APCI⁺) m/z 492 (M+H)⁺.

### Example 236: (2S)-6-chloro-4-oxo-N-[3-(2-{[6-(trifluoromethyl)pyridin-3-yl]oxy}acetamido)bicyclo[1.1.1]pentan-1-yl]-3,4-dihydro-2H-1-benzopyran-2-carboxamide (Compound 335)

The reaction and purification conditions described in Example 108E substituting the product of Example 126A for the product of Example 108A, and the product of Example 125A for the product of Example 108D gave the title compound. ¹H NMR (400 MHz, DMSO-d₆) δ ppm 8.96 (s, 1H), 8.83 (s, 1H), 8.46 (d, J=2.9 Hz, 1H), 7.87 (d, J=8.8 Hz, 1H), 7.68-7.61 (m, 2H), 7.57 (dd, J=8.7, 2.9 Hz, 1H), 7.20-7.13 (m, 1H), 5.09 (t, J=7.1 Hz, 1H), 4.66 (s, 2H), 2.95 (d, J=7.0 Hz, 2H), 2.26 (s, 6H); MS (APCI⁺) m/z 510 (M+H)⁺.

### Example 237: 6-chloro-N-{3-[2-(4-chloro-3-fluorophenoxy)acetamido]bicyclo[1.1.1]pentan-1-yl}-4-[(2,2,2-trifluoroethyl)amino]-3,4-dihydro-2H-1-benzopyran-2-carboxamide (Compound 336)

The methodologies described in Example 89 substituting 2,2,2-trifluoroethanamine for 2-((trimethylsilyl)oxy)ethanamine and purifying using preparative HPLC (Waters XBridge™ C₁₈ m OBD column, 30×100 mm, flow rate 40 mL/minute, 5-100% gradient of acetonitrile in 0.1% trifluoroacetic acid/water) gave the title compound. ¹H NMR (500 MHz, DMSO-d₆, dr 3:1) δ ppm 8.73 (s, 3H), 8.73 (s, 1H), 8.71 (s, 1H), 8.69 (s, 3H), 7.54-7.46 (m, 7H), 7.34 (d, J=2.7 Hz, 1H), 7.24 (dd, J=8.8, 2.7 Hz, 1H), 7.20 (dd, J=8.7, 2.7 Hz, 3H), 7.08 (dd, J=11.3, 2.9 Hz, 4H), 6.92 (dd, J=15.2, 8.7 Hz, 4H), 6.86 (ddd, J=8.9, 2.9, 1.1 Hz, 4H), 4.62 (dd, J=10.7, 2.7 Hz, 1H), 4.55 (dd, J=11.5, 2.3 Hz, 3H), 4.48 (d, J=1.8 Hz, 8H), 4.03 (td, J=9.9, 5.6 Hz, 3H), 3.77 (dt, J=7.1, 4.0 Hz, 1H), 3.32-3.23 (m, 5H), 2.93 (q, J=7.9 Hz, 4H), 2.47-2.39 (m, 3H), 2.29 (s, 18H), 2.28 (s, 6H), 2.24-2.12 (m, 1H), 1.81 (ddd, J=14.4, 10.7, 4.1 Hz, 1H), 1.66 (dt, J=13.1, 11.3 Hz, 3H); MS (APCI⁺) m/z 576 (M+H)⁺.

### Example 238: 6-chloro-N-{4-[2-(4-chloro-3-fluorophenoxy)acetamido]bicyclo[2.2.2]octan-1-yl}-4-hydroxy-3,4-dihydro-2H-1-benzopyran-2-carboxamide (Compound 337)

The methodologies described in Example 123 substituting the product of Example 227 for Example 214, reducing the reaction time to 5 minutes, and purifying by preparative HPLC (Waters XBridge™ C18 5 μm OBD column, 30×100 mm, flow rate 40 mL/minute, 5-100% gradient of acetonitrile in 0.1% trifluoroacetic acid/water) gave the title compound. ¹H NMR (400 MHz, DMSO-d₆) δ ppm 7.53-7.43 (m, 2H), 7.37 (dd, J=2.8, 1.0 Hz, 1H), 7.30 (s, 1H), 7.17 (ddd, J=8.7, 2.7, 0.7 Hz, 1H), 7.02 (dd, J=11.4, 2.8 Hz, 1H), 6.86 (d, J=8.8 Hz, 1H), 6.81 (ddd, J=9.0, 2.9, 1.2 Hz, 1H), 5.67 (d, J=5.0 Hz, 1H), 4.77 (s, 0.1H), 4.55 (dd, J=11.8, 2.3 Hz, 1H), 4.43 (s, 2H), 2.27 (ddd, J=12.8, 5.9, 2.3 Hz, 1H), 1.92 (s, 12H), 1.72 (ddd, J=13.0, 11.8, 10.7 Hz, 1H); MS (APCI⁺) m/z 520 (M−H₂O+H)⁺.

### Example 239: 6-chloro-N-{3-[2-(4-chloro-3-fluorophenoxy)acetamido]bicyclo[1.1.1]pentan-1-yl}-2-methyl-4-oxo-3,4-dihydro-2H-1-benzopyran-2-carboxamide (Compound 338)

To a mixture of the product of Example 141 (0.113 g, 0.229 mmol) in acetic acid (0.69 mL) and chloroform (0.01 mL) was added chromium trioxide (0.053 g, 0.53 mmol) dissolved in water (1 mL) at 0° C. The reaction mixture was allowed to stir at ambient temperature for 1 hour and then was heated to 50° C. overnight. Additional chromium trioxide (0.053 g, 0.53 mmol) was added to the reaction mixture, which was then heated to 70° C. for 7 hours and 50° C. for 3 days. Additional chromium trioxide (0.053 g, 0.53 mmol) and acetic acid (0.69 mL) were added to the reaction mixture, which was then heated to 70° C. for 5 hours. Although the conversion was incomplete, the reaction mixture was diluted with H₂O (1 mL) and extracted with ethyl acetate (3×5 mL). The combined organic extracts were dried over Na₂SO₄, filtered, and concentrated. The crude material was purified preparative HPLC (Waters XBridge™ C18 5 μm OBD column, 30×100 mm, flow rate 40 mL/minute, 5-100% gradient of acetonitrile in 0.1% trifluoroacetic acid/water) to give the title compound (0.008 g, 0.016 mmol, 7% yield). ¹H NMR (500 MHz, DMSO-d₆) δ ppm 8.78 (s, 1H), 8.67 (s, 1H), 7.66 (dd, J=8.8, 2.8 Hz, 1H), 7.63 (d, J=2.6 Hz, 1H), 7.48 (t, J=8.9 Hz, 1H), 7.17 (d, J=8.8 Hz, 1H), 7.06 (dd, J=11.4, 2.8 Hz, 1H), 6.83 (ddd, J=9.0, 2.9, 1.2 Hz, 1H), 4.45 (s, 2H), 3.12 (d, J=16.7 Hz, 1H), 2.91 (d, J=16.8 Hz, 1H), 2.13 (s, 6H), 1.56 (s, 3H); MS (APCI⁺) m/z 507 (M+H)⁺.

### Example 240: 6-chloro-N-{3-[2-(4-chloro-3-fluorophenoxy)acetamido]bicyclo[1.1.1]pentan-1-yl}-4-{[1-(hydroxymethyl)cyclopropyl]amino}-3,4-dihydro-2H-1-benzopyran-2-carboxamide (Compound 339)

The methodologies described in Example 89 substituting (1-aminocyclopropyl)methanol for 2-((trimethylsilyl)oxy)ethanamine and purifying using preparative HPLC [Waters XBridge™ C18 5 μm OBD column, 30×100 mm, flow rate 40 mL/minute, 5-100% gradient of acetonitrile in buffer (0.025 M aqueous ammonium bicarbonate, adjusted to pH 10 with ammonium hydroxide)] gave the title compound as the minor product (26% yield), along with the product of Example 91. ¹H NMR (400 MHz, DMSO-d₆, dr 1:1) δ ppm 8.71 (s, 2H), 8.67 (s, 1H), 8.62 (s, 1H), 7.54-7.44 (m, 3H), 7.32 (d, J=2.7 Hz, 1H), 7.20 (dd, J=8.7, 2.6 Hz, 1H), 7.15 (ddd, J=8.7, 2.7, 0.7 Hz, 1H), 7.08 (dd, J=11.4, 2.8 Hz, 2H), 6.93-6.82 (m, 4H), 4.68 (s, 1H), 4.57 (ddd, J=13.1, 11.1, 2.4 Hz, 2H), 4.48 (s, 4H), 4.09 (d, J=13.6 Hz, 2H), 3.74 (d, J=11.1 Hz, 1H), 3.42 (d, J=3.9 Hz, 2H), 3.18 (d, J=11.0 Hz, 1H), 2.59-2.52 (m, 1H), 2.49-2.40 (m, 2H), 2.33 (t, J=2.8 Hz, 1H), 2.28 (s, 12H), 1.78-1.60 (m, 2H), 0.65-0.30 (m, 8H); MS (APCI⁺) m/z 564 (M+H)⁺.

### Example 241: rac-(2R,4R)-6-chloro-N-{3-[2-(4-chloro-3-fluorophenoxy)acetamido]bicyclo[1.1.1]pentan-1-yl}-4-methoxy-3,4-dihydro-2H-1-benzopyran-2-carboxamide (Compound 340)

The methodologies described in Example 89 substituting 3,3-difluorocyclobutanamine hydrochloride for 2-((trimethylsilyl)oxy)ethanamine gave the title compound as a minor product (7% yield) and single diastereomer, along with the product of Example 91 and Example 113. ¹H NMR (500 MHz, DMSO-d₆) δ ppm 8.77 (d, J=2.9 Hz, 1H), 8.67 (d, J=16.5 Hz, 1H), 7.49 (t, J=8.9 Hz, 1H), 7.29 (d, J=2.7 Hz, 1H), 7.24 (dd, J=8.7, 2.8 Hz, 1H), 7.07 (dd, J=11.4, 2.9 Hz, 1H), 6.92 (d, J=8.8 Hz, 1H), 6.88-6.82 (m, 1H), 4.62 (dd, J=10.0, 3.1 Hz, 1H), 4.55 (dd, J=9.0, 5.1 Hz, 1H), 4.48 (s, 2H), 3.37 (s, 2H), 2.53-2.45 (m, 1H), 2.27 (d, J=6.9 Hz, 6H), 1.85 (dt, J=13.2, 9.4 Hz, 1H); MS (APCI⁺) m/z 477 (M−CH₃OH+H)⁺.

### Example 242: N-{3-[2-(4-chloro-3-fluorophenoxy)acetamido]bicyclo[1.1.1]pentan-1-yl}-6-methyl-4-oxo-4H-1-benzopyran-2-carboxamide (Compound 341)

### Example 242A: ethyl 6-methyl-4-oxo-4H-chromene-2-carboxylate

The methodologies described in Example 131A substituting 1-(2-hydroxy-5-methylphenyl)ethanone for 1-(2-hydroxy-4-(trifluoromethyl)phenyl)ethanone gave the title compound. ¹H NMR (400 MHz, DMSO-d₆) δ ppm 7.84 (dt, J=2.3, 0.8 Hz, 1H), 7.70 (dd, J=8.8, 2.2 Hz, 1H), 7.65 (d, J=8.6 Hz, 1H), 6.93 (s, 1H), 4.40 (q, J=7.1 Hz, 2H), 2.44 (s, 3H), 1.35 (t, J=7.1 Hz, 3H); MS (ESI⁺) m/z 233 (M+H)⁺.

### Example 242B: 6-methyl-4-oxo-4H-chromene-2-carboxylic Acid

The methodologies described in Example 131B substituting Example 242A for Example 131A gave the title compound. ¹H NMR (400 MHz, DMSO-d₆) δ ppm 7.87-7.81 (m, 1H), 7.69 (dd, J=8.8, 2.2 Hz, 1H), 7.63 (d, J=8.6 Hz, 1H), 6.89 (s, 1H), 2.44 (s, 3H); MS (ESI⁺) m/z 205 (M+H)⁺.

### Example 242C: N-{3-[2-(4-chloro-3-fluorophenoxy)acetamido]bicyclo[1.1.1]pentan-1-yl}-6-methyl-4-oxo-4H-1-benzopyran-2-carboxamide

The methodologies described in Example 131C substituting Example 242B for Example 131B gave the title compound. ¹H NMR (400 MHz, DMSO-d₆) δ ppm 8.86 (s, 1H), 7.83 (s, 1H), 7.71 (dd, J=8.7, 2.2 Hz, 1H), 7.65 (d, J=8.6 Hz, 1H), 7.48 (t, J=8.9 Hz, 1H), 7.06 (dd, J=11.3, 2.8 Hz, 1H), 6.86 (dd, J=9.1, 2.8 Hz, 1H), 6.80 (s, 1H), 4.49 (s, 2H), 2.43 (s, 3H), 2.39 (s, 6H).

### Example 243: (2S)—N-{3-[2-(3,4-dichlorophenoxy)acetamido]bicyclo[1.1.1]pentan-1-yl}-6,7-difluoro-4-oxo-3,4-dihydro-2H-1-benzopyran-2-carboxamide (Compound 342)

The reaction and purification conditions described in Example 108E substituting the product of Example 109A for the product of Example 108D gave the title compound. ¹H NMR (500 MHz, DMSO-d₆) δ ppm 8.95 (s, 1H), 8.74 (s, 1H), 7.69 (dd, J=10.3, 9.1 Hz, 1H), 7.55 (d, J=8.9 Hz, 1H), 7.30-7.23 (m, 2H), 6.98 (dd, J=8.9, 2.9 Hz, 1H), 5.11 (dd, J=7.5, 6.7 Hz, 1H), 4.49 (s, 2H), 2.97-2.92 (m, 2H), 2.26 (s, 6H); MS (APCI⁺) m/z 511 (M+H)⁺.

### Example 244: rac-(2R,4S)-6-chloro-N-{3-[2-(4-chloro-3-fluorophenoxy)acetamido]bicyclo[1.1.1]pentan-1-yl}-4-{[1-(hydroxymethyl)cyclopropyl]amino}-3,4-dihydro-2H-1-benzopyran-2-carboxamide (Compound 343)

Example 240 was purified by chiral SFC (supercritical fluid chromatography) using a Regis Technologies, Inc. Whelk-O®1 column eluting with 40% CH₃OH with 0.1% diethylamine in CO₂ with a flow rate of 80 g/minute and back pressure of 100 bar to give the title compound (second isomer eluted out of the column, 0.005 g, 0.009 mmol, 22% yield). The stereochemistry of this title compound was arbitrarily assigned. ¹H NMR (400 MHz, DMSO-d₆) δ ppm 8.72 (s, 1H), 8.67 (s, 1H), 7.50 (t, J=8.9 Hz, 1H), 7.32 (d, J=2.6 Hz, 1H), 7.20 (dd, J=8.8, 2.6 Hz, 1H), 7.08 (dd, J=11.4, 2.8 Hz, 1H), 6.90 (d, J=8.8 Hz, 1H), 6.86 (ddd, J=9.0, 2.8, 1.2 Hz, 1H), 4.70 (t, J=5.4 Hz, 1H), 4.58 (dd, J=11.1, 2.4 Hz, 1H), 4.48 (s, 2H), 4.10-4.03 (m, 1H), 3.74 (dd, J=11.7, 5.6 Hz, 1H), 3.18 (dd, J=11.5, 5.1 Hz, 1H), 2.42 (s, 1H), 2.31 (dd, J=13.9, 2.9 Hz, 1H), 2.28 (s, 6H), 1.71 (ddd, J=14.3, 11.2, 3.7 Hz, 1H), 0.60 (ddd, J=9.7, 5.9, 4.0 Hz, 1H), 0.49 (ddd, J=9.8, 6.1, 3.9 Hz, 1H), 0.41 (ddd, J=9.5, 5.9, 3.8 Hz, 1H), 0.34 (dt, J=10.3, 5.0 Hz, 1H); MS (APCI⁺) m/z 564 (M+H)⁺.

### Example 245: N-[(3S)-4-{[(2R,4R)-6-chloro-4-hydroxy-3,4-dihydro-2H-1-benzopyran-2-carbonyl]amino}-3-hydroxybicyclo[2.2.2]octan-1-yl]-4-methyl-1,3-thiazole-2-carboxamide (Compound 344)

### Example 245A: (S)-tert-butyl (2-hydroxy-4-(4-methylthiazole-2-carboxamido)bicyclo[2.2.2]octan-1-yl)carbamate

4-Methylthiazole-2-carboxylic acid (CombiBlocks, 0.208 g, 1.455 mmol), the product of Example 85C (0.426 g, 1.455 mmol) and triethylamine (0.608 mL) were combined with N,N-dimethylformamide (7 mL) and stirred at ambient temperature. 1-[Bis(dimethylamino)methylene]-1H-1,2,3-triazolo[4,5-b]pyridinium 3-oxid hexafluorophosphate (HATU, 0.609 g, 1.60 mmol) was added in one portion. The resulting mixture was stirred for 1 hour. Water (0.5 mL) was added, and the resulting solution was filtered through a glass microfiber frit and purified by preparative HPLC [YMC TriArt™ C18 Hybrid 5 μm column, 50×100 mm, flow rate 140 mL/minute, 5-100% gradient of acetonitrile in buffer (0.025 M aqueous ammonium bicarbonate, adjusted to pH 10 with ammonium hydroxide)] to give the title compound (0.30 g, 0.79 mmol, 54% yield). MS (ESI⁺) m/z 326 (M−C(CH₃)₃+H)⁺.

### Example 245B: (S)—N-(4-amino-3-hydroxybicyclo[2.2.2]octan-1-yl)-4-methylthiazole-2-carboxamide, trifluoroacetic Acid

The reaction and purification conditions described in Example 128B substituting the product of Example 245A for the product of Example 128A gave the title compound. MS (ESI⁺) m/z 282 (M+H)⁺.

### Example 245C: (2R,4R)-6-chloro-4-hydroxychroman-2-carboxylic Acid

The product of Example 124A was treated using the procedure described in Example 108F. After quenching with ammonium chloride, the resulting reaction mixture was combined with diatomaceous earth and then concentrated under reduced pressure to give a free flowing powder. The powder was directly applied to reversed-phase flash chromatography [Interchim® PuriFlash® C18XS 30 μm 175 g column, flow rate 100 mL/minute, 5-100% gradient of acetonitrile in buffer (0.1% trifluoroacetic acid)] to give the title compound. MS (APCI⁻) m/z 227 (M−H)⁻.

### Example 245D: N-[(3S)-4-{[(2R,4R)-6-chloro-4-hydroxy-3,4-dihydro-2H-1-benzopyran-2-carbonyl]amino}-3-hydroxybicyclo[2.2.2]octan-1-yl]-4-methyl-1,3-thiazole-2-carboxamide

Hunig's base (0.124 mL) was added to a solution of the product of Example 245C (16 mg, 0.07 mmol) and the product of Example 245B (28 mg, 0.07 mmol) in N,N-dimethylformamide (2 mL). While stirring at ambient temperature, 1-propanephosphonic anhydride (50 weight % solution in N,N-dimethylformamide, 0.050 mL) was added dropwise over a period of 2 minutes. After stirring for 1 hour, more 1-propanephosphonic anhydride (50 weight % solution in N,N-dimethylformamide, 0.020 mL) and the product of Example 245C (8 mg, 0.035 mmol) were added. The resulting reaction mixture was stirred for another 30 minutes, and then partitioned between dichloromethane (2×25 mL) and aqueous sodium carbonate (1.0 M, 20 mL). The organic layers were combined, dried over anhydrous sodium sulfate and concentrated under reduced pressure. The resulting residue was purified by preparative HPLC [YMC TriArt™ C18 Hybrid 5 μm column, 50×100 mm, flow rate 140 mL/minute, 5-100% gradient of acetonitrile in buffer (0.025 M aqueous ammonium bicarbonate, adjusted to pH 10 with ammonium hydroxide)] to give the title compound (4 mg, 8.1 μmol, 12% yield). ¹H NMR (500 MHz, DMSO-d₆) δ ppm 7.73 (s, 1H), 7.57-7.55 (m, 1H), 7.38 (dd, J=2.7, 1.0 Hz, 1H), 7.26-7.18 (m, 2H), 6.86 (d, J=8.7 Hz, 1H), 5.70 (d, J=6.0 Hz, 1H), 5.19 (d, J=4.6 Hz, 1H), 4.79 (dt, J=11.0, 5.5 Hz, 1H), 4.65-4.59 (m, 1H), 4.09-4.05 (m, 1H), 2.44-2.40 (m, 4H), 2.36 (ddd, J=13.0, 5.8, 2.5 Hz, 1H), 2.25-2.17 (m, 1H), 2.12-2.03 (m, 1H), 1.99-1.82 (m, 7H), 1.73 (ddd, J=13.1, 11.5, 10.4 Hz, 1H); MS (APCI⁺) m/z 492 (M+H)⁺.

### Example 246: 7-chloro-N-{3-[2-(4-chloro-3-fluorophenoxy)acetamido]bicyclo[1.1.1]pentan-1-yl}-3,4-dihydro-2H-1-benzopyran-3-carboxamide (Compound 345)

The methodologies described in Example 122 substituting 7-chlorochroman-3-carboxylic acid for 6,7-dimethoxy-4-oxo-4H-chromene-2-carboxylic acid gave the title compound. ¹H NMR (400 MHz, DMSO-d₆) δ ppm 8.73 (s, 1H), 8.71 (s, 1H), 7.49 (t, J=8.9 Hz, 1H), 7.13 (d, J=8.3 Hz, 1H), 7.07 (dd, J=11.4, 2.8 Hz, 1H), 6.92-6.81 (m, 3H), 4.47 (s, 2H), 4.31 (ddd, J=10.8, 3.6, 1.8 Hz, 1H), 3.90 (dd, J=10.8, 9.6 Hz, 1H), 2.92-2.76 (m, 2H), 2.79-2.68 (m, 1H), 2.24 (s, 6H).; MS (APCI⁺) m/z 479 (M+H)⁺.

### Example 247: N-{3-[2-(4-chloro-3-fluorophenoxy)acetamido]bicyclo[1.1.1]pentan-1-yl}-6-hydroxy-4-oxo-4H-1-benzopyran-2-carboxamide (Compound 346)

### Example 247A: 6-hydroxy-4-oxo-4H-chromene-2-carboxylic Acid

The methodologies described in Example 131B substituting ethyl 6-hydroxy-4-oxo-4H-chromene-2-carboxylate (European Patent Application EP1473293 Al, 2004, Page 25) for Example 131A gave the title compound. ¹H NMR (400 MHz, DMSO-d₆) δ ppm 10.16 (s, 1H), 7.65-7.58 (m, 1H), 7.34-7.27 (m, 2H), 6.83 (s, 1H); MS (ESI⁺) m/z 207 (M+H)⁺.

### Example 247B: N-{3-[2-(4-chloro-3-fluorophenoxy)acetamido]bicyclo[1.1.1]pentan-1-yl}-6-hydroxy-4-oxo-4H-1-benzopyran-2-carboxamide

The methodologies described in Example 131C substituting Example 247A for Example 131B gave the title compound. ¹H NMR (400 MHz, DMSO-d₆) δ ppm 7.65 (d, J=8.8 Hz, 1H), 7.50 (t, J=8.8 Hz, 1H), 7.38-7.30 (m, 2H), 7.07 (dd, J=11.3, 2.9 Hz, 1H), 6.88 (dd, J=9.0, 2.8 Hz, 1H), 6.77 (s, 1H), 4.50 (s, 2H), 2.40 (s, 6H).

### Example 248: N-{3-[2-(4-chloro-3-fluorophenoxy)acetamido]bicyclo[1.1.1]pentan-1-yl}-6-fluoro-4-hydroxy-7-methyl-3,4-dihydro-2H-1-benzopyran-2-carboxamide (Compound 347)

The methodologies described in Example 122 substituting 6-fluoro-4-hydroxy-7-methylchroman-2-carboxylic acid for 6,7-dimethoxy-4-oxo-4H-chromene-2-carboxylic acid gave the title compound. ¹H NMR (500 MHz, DMSO-d₆) δ ppm 8.73 (s, 1H), 8.64 (s, 1H), 7.50 (t, J=8.9 Hz, 1H), 7.11-7.03 (m, 2H), 6.86 (ddd, J=9.0, 2.8, 1.2 Hz, 1H), 6.76 (dd, J=6.7, 0.9 Hz, 1H), 5.60 (d, J=6.2 Hz, 1H), 4.76 (t, J=5.6 Hz, 1H), 4.52 (dd, J=12.0, 2.2 Hz, 1H), 4.48 (s, 2H), 2.32 (ddd, J=13.1, 6.1, 2.4 Hz, 1H), 2.28 (s, 6H), 2.18-2.13 (m, 3H), 1.71-1.60 (m, 1H); MS (APCI⁺) m/z 475 (M−H₂O+H)⁺.

### Example 249: (2S)-6-chloro-N-{3-[2-(4-chloro-3-fluorophenoxy)acetamido]bicyclo[1.1.1]pentan-1-yl}-4-(2-hydroxy-2-methylpropyl)-3,4-dihydro-2H-1,4-benzoxazine-2-carboxamide (Compound 348)

To a solution of the product of Example 112 (0.053 g, 0.096 mmol) in tetrahydrofuran (0.27 mL) at 0° C. was added methylmagnesium bromide (0.102 mL, 0.306 mmol, 3 M in tetrahydrofuran). The reaction mixture stirred for 4 days as additional methylmagnesium bromide (0.1 mL, 0.306 mmol, 3 M in tetrahydrofuran) was added three times, as the reaction mixture was presumed to be incomplete due to the similar retention time and mass of the starting material and product by liquid chromatography-mass spectrometry (LCMS). The reaction mixture was then quenched with H₂O (1 mL) and dilute HCl (1 M, 0.5 mL) and then was extracted with dichloromethane (3×5 mL). The combined organic fractions were dried (Na₂SO₄), and concentrated. The residue was purified by preparative HPLC (Waters XBridge™ C18 5 μm OBD column, 30×100 mm, flow rate 40 mL/minute, 5-100% gradient of acetonitrile in 0.1% trifluoroacetic acid/water) to give the title compound (0.005 g, 0.0091 mmol, 9% yield). ¹H NMR (501 MHz, DMSO-d₆) δ ppm 8.71 (s, 2H), 7.49 (t, J=8.9 Hz, 1H), 7.12-7.04 (m, 1H), 6.90 (d, J=2.4 Hz, 1H), 6.85 (ddd, J=9.1, 2.8, 1.2 Hz, 1H), 6.77 (d, J=8.4 Hz, 1H), 6.53 (dd, J=8.4, 2.4 Hz, 1H), 4.50-4.45 (m, 1H), 4.47 (s, 3H), 3.58 (dd, J=12.7, 2.8 Hz, 1H), 3.21 (d, J=14.8 Hz, 1H), 3.09 (d, J=14.9 Hz, 1H), 2.25 (s, 6H), 2.07 (s, 1H), 1.13 (s, 3H), 1.11 (s, 3H).; MS (APCI⁺) m/z 553 (M+H)⁺.

### Example 250: 6-chloro-N-{3-[2-(4-chloro-3-fluorophenoxy)acetamido]bicyclo[1.1.1]pentan-1-yl}-4-[(oxetan-3-yl)amino]-3,4-dihydro-2H-1-benzopyran-2-carboxamide (Compound 349)

The methodologies described in Example 89 substituting oxetan-3-amine for 2-((trimethylsilyl)oxy)ethanamine and purifying using preparative HPLC [Waters XBridge™ C18 5 μm OBD column, 30×100 mm, flow rate 40 mL/minute, 5-100% gradient of acetonitrile in buffer (0.025 M aqueous ammonium bicarbonate, adjusted to pH 10 with ammonium hydroxide)] gave the title compound. ¹H NMR (501 MHz, DMSO-d₆, dr 1:1) δ ppm 8.72 (s, 2H), 8.69 (s, 1H), 8.64 (s, 1H), 7.53-7.46 (m, 3H), 7.27 (d, J=2.6 Hz, 1H), 7.25-7.16 (m, 2H), 7.07 (dd, J=11.4, 2.9 Hz, 2H), 6.95-6.84 (m, 3H), 6.85 (dd, J=2.9, 1.2 Hz, 1H), 4.70-4.58 (m, 5H), 4.51 (dd, J=11.2, 2.5 Hz, 1H), 4.48 (s, 4H), 4.37 (q, J=6.7 Hz, 3H), 4.31 (t, J=6.2 Hz, 1H), 4.13-3.94 (m, 2H), 3.95-3.87 (m, 1H), 3.65 (s, 1H), 3.03-2.97 (m, 1H), 2.95 (t, J=9.0 Hz, 1H), 2.28 (s, 12H), 2.17 (ddd, J=13.0, 5.4, 2.5 Hz, 1H), 2.03-1.97 (m, 1H), 1.76-1.66 (m, 1H), 1.59-1.49 (m, 1H); MS (APCI⁺) m/z 550 (M+H)⁺.

### Example 251: 6-chloro-N-{3-[2-(4-chloro-3-fluorophenoxy)acetamido]bicyclo[1.1.1]pentan-1-yl}-2-fluoro-4-hydroxy-3,4-dihydro-2H-1-benzopyran-2-carboxamide (Compound 350)

### Example 251A: methyl 6-chloro-4-hydroxychroman-2-carboxylate

To a solution of 6-chloro-4-hydroxychroman-2-carboxylic acid (1.4 g, 6.1 mmol) in methanol (61 mL) was added Amberlyst® 15 hydrogen form (0.7 g, 50 weight %), and the mixture was allowed to stir at ambient temperature overnight. Then the reaction mixture was filtered over a bed of sand and diatomaceous earth, and the filtrate was concentrated in vacuo to afford the title compound (1.48 g, 6.12 mmol, quantitative yield) which was carried forward without purification. ¹H NMR (400 MHz, DMSO-d₆) δ ppm 7.32 (dd, J=2.6, 0.8 Hz, 1H), 7.21 (dd, J=8.7, 2.7 Hz, 1H), 6.87 (d, J=8.7 Hz, 1H), 5.55 (d, J=4.7 Hz, 1H), 5.00 (dd, J=7.5, 4.1 Hz, 1H), 4.73 (dt, J=6.9, 4.7 Hz, 1H), 3.66 (s, 3H), 2.30 (dt, J=13.6, 4.5 Hz, 1H), 2.08 (dt, J=13.9, 7.2 Hz, 1H).

### Example 251B: methyl 4-((tert-butyldimethylsilyl)oxy)-6-chlorochroman-2-carboxylate

To a solution of Example 251A (1.49 g, 6.12 mmol) in tetrahydrofuran (24 mL) at 0° C. was added tert-butyldimethylchlorosilane (2.031 g, 13.5 mmol) followed by imidazole (1.00 g, 14.70 mmol). The cooling bath was removed, and the flask was allowed to warm to ambient temperature overnight. Then additional tert-butyldimethylchlorosilane (2.031 g, 13.5 mmol) and imidazole (1.00 g, 14.70 mmol) were added to the reaction mixture. After stirring at ambient temperature for another 4 hours, the reaction mixture was diluted with water (80 mL), extracted with diethyl ether (3×25 mL), and concentrated. A portion of the residue was purified by preparative HPLC [Waters XBridge™ C18 5 μm OBD column, 30×100 mm, flow rate 40 mL/minute, 5-100% gradient of acetonitrile in buffer (0.025 M aqueous ammonium bicarbonate, adjusted to pH 10 with ammonium hydroxide)] to give the title compound. ¹H NMR (500 MHz, DMSO-d₆) δ ppm 7.25 (dd, J=8.7, 2.6 Hz, 1H), 7.16 (dd, J=2.7, 0.7 Hz, 1H), 6.91 (d, J=8.8 Hz, 1H), 5.07 (dd, J=6.5, 4.6 Hz, 1H), 4.97-4.92 (m, 1H), 3.66 (s, 3H), 2.35 (dt, J=13.9, 4.6 Hz, 1H), 2.15 (dt, J=13.9, 6.2 Hz, 1H), 0.87 (s, 9H), 0.16 (s, 3H), 0.15 (s, 3H).

### Example 251C: methyl 4-((tert-butyldimethylsilyl)oxy)-6-chloro-2-fluorochroman-2-carboxylate

A solution of Example 251B (0.25 g, 0.70 mmol) and N-fluoro-N-(phenylsulfonyl)benzenesulfonamide (0.442 g, 1.40 mmol) in tetrahydrofuran (1.8 mL) was cooled to −78° C. Then lithium bis(trimethylsilyl)amide (1.05 mL, 1.05 mmol, 1 M in tetrahydrofuran) was added dropwise. The reaction mixture was stirred at −78° C. for 8 hours, quenched with 0.5 M HCl (75 mL), and extracted with ethyl ether (3×50 mL). The combined organic phases were concentrated under reduced pressure. The residue was diluted with N,N-dimethylformamide/water and purified by preparative HPLC [Waters XBridge™ C18 5 μm OBD column, 30×100 mm, flow rate 40 mL/minute, 5-100% gradient of acetonitrile in buffer (0.025 M aqueous ammonium bicarbonate, adjusted to pH 10 with ammonium hydroxide)] to give the title compound (0.040 g, 0.107 mmol, 15% yield, dr 14:1) and recovered starting material. ¹H NMR (400 MHz, DMSO-d₆, dr 14.3:1) δ ppm 7.19 (ddd, J=8.8, 2.7, 0.7 Hz, 1H), 7.14 (dd, J=2.6, 1.0 Hz, 1H), 7.07 (dd, J=8.7, 2.7 Hz, 0.06H), 6.98 (d, J=2.6 Hz, 0.06H), 6.89 (d, J=8.7 Hz, 1H), 6.73 (d, J=8.8 Hz, 0.07H), 4.86 (dd, J=11.0, 5.7 Hz, 1H), 4.77 (t, J=5.2 Hz, 0.06H), 3.67 (s, 3H), 3.49 (s, 0.2H), 2.57-2.46 (m, 1H), 2.18-1.95 (m, 1H), 0.75 (s, 9H), 0.69 (d, J=5.4 Hz, 0.6H), 0.04 (s, 3H), −0.00 (s, 3H), −0.02 (s, 0.2H), −0.03 (s, 0.2H).

### Example 251D: 4-((tert-butyldimethylsilyl)oxy)-6-chloro-2-fluorochroman-2-carboxylic Acid

To a solution of Example 251C (0.020 g, 0.054 mmol) in methanol (0.07 mL) and water (0.04 mL) was added NaOH (0.11 mL, 0.27 mmol, 2.5 N). The reaction mixture was stirred at ambient temperature for 30 minutes, was diluted with 1 drop of 10% HCl, and was concentrated to give the title compound (0.020 g, quantitative yield), which was carried forward without purification. ¹H NMR (400 MHz, DMSO-d₆) δ ppm 7.14-7.05 (m, 2H), 6.74 (d, J=9.4 Hz, 1H), 4.75 (dd, J=11.7, 6.0 Hz, 1H), 2.25 (ddd, J=13.7, 6.2, 2.7 Hz, 1H), 1.94-1.75 (m, 1H), 0.79 (s, 9H), 0.05 (s, 3H), −0.00 (s, 3H).

### Example 251E: 6-chloro-N-{3-[2-(4-chloro-3-fluorophenoxy)acetamido]bicyclo[1.1.1]pentan-1-yl}-2-fluoro-4-hydroxy-3,4-dihydro-2H-1-benzopyran-2-carboxamide

The methodologies described in Example 122 substituting Example 251D for 6,7-dimethoxy-4-oxo-4H-chromene-2-carboxylic acid and purifying by preparative HPLC [Waters XBridge™ C18 5 μm OBD column, 30×100 mm, flow rate 40 mL/minute, 5-100% gradient of acetonitrile in buffer (0.025 M aqueous ammonium bicarbonate, adjusted to pH 10 with ammonium hydroxide)] gave the title compound. ¹H NMR (500 MHz, DMSO-d₆) δ ppm 9.30 (s, 1H), 8.82 (s, 1H), 7.53-7.45 (m, 2H), 7.33 (dd, J=8.7, 2.6 Hz, 1H), 7.08 (dd, J=11.3, 2.9 Hz, 1H), 7.04 (d, J=8.7 Hz, 1H), 6.87 (dd, J=9.2, 2.9 Hz, 1H), 4.80 (dd, J=11.8, 6.1 Hz, 1H), 4.48 (s, 2H), 2.57 (dd, J=6.1, 3.3 Hz, 1H), 2.31 (s, 6H), 2.08-1.92 (m, 1H); MS (APCI⁺) m/z 495 (M−H₂O+H)⁺.

### Example 252: (2R,4R)-6-chloro-N-(3-{2-[(4-fluoro-1H-indazol-6-yl)oxy]acetamido}bicyclo[1.1.1]pentan-1-yl)-4-hydroxy-3,4-dihydro-2H-1-benzopyran-2-carboxamide (Compound 351)

### Example 252A: (R)-6-chloro-N-(3-(2-((4-fluoro-1H-indazol-6-yl)oxy)acetamido)bicyclo[1.1.1]pentan-1-yl)-4-oxochroman-2-carboxamide

The reaction and purification conditions described in Example 108E substituting the product of Example 128B for the product of Example 108A, and 2-((4-fluoro-1H-indazol-6-yl)oxy)acetic acid (prepared as described in International Patent Publication WO2017/193034, 2017, Al) for the product of Example 108D gave the title compound. MS (APCI⁺) m/z 499 (M+H)⁺.

### Example 252B: (2R,4R)-6-chloro-N-(3-{2-[(4-fluoro-1H-indazol-6-yl)oxy]acetamido}bicyclo[1.1.1]pentan-1-yl)-4-hydroxy-3,4-dihydro-2H-1-benzopyran-2-carboxamide

The reaction and purification conditions described in Example 108F substituting the product of Example 252A for the product of Example 108E gave the title compound. ¹H NMR (400 MHz, DMSO-d₆) δ ppm 8.75 (s, 1H), 8.68 (s, 1H), 8.05 (d, J=0.9 Hz, 1H), 7.38 (d, J=2.6 Hz, 1H), 7.20 (dd, J=8.7, 2.7 Hz, 1H), 6.88 (d, J=8.7 Hz, 1H), 6.81-6.75 (m, 1H), 6.68 (dd, J=11.6, 1.8 Hz, 1H), 5.72 (br s, 1H), 4.80 (dd, J=10.7, 5.9 Hz, 1H), 4.60 (dd, J=11.9, 2.3 Hz, 1H), 4.52 (s, 2H), 2.35 (ddd, J=13.0, 5.9, 2.4 Hz, 1H), 2.30 (s, 6H), 1.70 (td, J=12.6, 10.8 Hz, 1H); MS (ESI⁺) m/z 483 (M−H₂O+H)⁺.

### Example 253: N-[(2S)-4-{[(2R)-6-chloro-4-oxo-3,4-dihydro-2H-1-benzopyran-2-carbonyl]amino}-2-hydroxybicyclo[2.2.2]octan-1-yl]-5-(difluoromethyl)pyrazine-2-carboxamide (Compound 352)

The product of Example 175A (26.7 mg, 0.057 mmol) and trifluoroacetic acid (0.5 mL) were combined and stirred at ambient temperature for 30 minutes, and then the mixture was concentrated under reduced pressure. To the residue was added N,N-dimethylformamide (2 mL), 5-(difluoromethyl)pyrazine-2-carboxylic acid (Enamine, 10 mg, 0.057 mmol) and triethylamine (0.040 mL) sequentially. While the mixture was stirring, 1-[bis(dimethylamino)methylene]-1H-1,2,3-triazolo[4,5-b]pyridinium 3-oxid hexafluorophosphate (HATU, 26.2 mg, 0.069 mmol) was added. After 1 hour, the reaction mixture was partitioned between dichloromethane (2×25 mL) and aqueous sodium carbonate (1.0 M, 20 mL). The organic layers were combined and dried over anhydrous sodium sulfate and concentrated under reduced pressure. The residue was purified by preparative HPLC [Waters XBridge™ C18 5 μm OBD column, 30×100 mm, flow rate 40 mL/minute, 5-100% gradient of acetonitrile in buffer (0.025 M aqueous ammonium bicarbonate, adjusted to pH 10 with ammonium hydroxide)] to give the title compound. ¹H NMR (500 MHz, DMSO-d₆) δ ppm 9.25 (d, J=1.4 Hz, 1H), 9.00 (d, J=1.4 Hz, 1H), 8.14 (s, 1H), 7.79 (s, 1H), 7.66-7.58 (m, 2H), 7.21 (t, J=54.0 Hz, 1H), 7.16 (d, J=8.7 Hz, 1H), 5.32 (d, J=5.2 Hz, 1H), 5.07 (dd, J=8.3, 4.9 Hz, 1H), 4.07-4.00 (m, 1H), 3.01-2.87 (m, 3H), 2.30 (td, J=10.3, 9.4, 5.4 Hz, 1H), 2.11-2.04 (m, 1H), 1.98-1.90 (m, 2H), 1.87-1.76 (m, 5H); MS (ESI⁺) m/z 520 (M+H)⁺.

### Example 254: N-[(2S)-4-{[(2R,4R)-6-chloro-4-hydroxy-3,4-dihydro-2H-1-benzopyran-2-carbonyl]amino}-2-hydroxybicyclo[2.2.2]octan-1-yl]-5-(difluoromethyl)pyrazine-2-carboxamide (Compound 353)

The reaction and purification conditions described in Example 108F substituting the product of Example 253B for the product of Example 108E gave the title compound. ¹H NMR (400 MHz, DMSO-d₆) δ ppm 9.26 (d, J=1.4 Hz, 1H), 9.00 (d, J=1.3 Hz, 1H), 8.15 (s, 1H), 7.40 (s, 1H), 7.37 (dd, J=2.7, 0.9 Hz, 1H), 7.20 (t, J=54.0 Hz, 1H), 7.18 (dd, J=8.6, 2.6 Hz, 1H), 6.87 (d, J=8.7 Hz, 1H), 5.67 (br s, 1H), 5.34 (br s, 1H), 4.78 (dd, J=10.6, 5.9 Hz, 1H), 4.56 (dd, J=11.8, 2.2 Hz, 1H), 4.11-4.02 (m, 1H), 2.55-2.51 (m, 1H), 2.38 (ddd, J=12.8, 9.4, 2.7 Hz, 1H), 2.28 (ddd, J=13.0, 6.0, 2.3 Hz, 1H), 2.15-1.93 (m, 3H), 1.93-1.80 (m, 5H), 1.74 (ddd, J=12.9, 11.9, 10.7 Hz, 1H); MS (APCI⁺) m/z 505 (M−H₂O+H)⁺.

### Example 255: N-[(2S)-4-{[(2R)-6-chloro-4-oxo-3,4-dihydro-2H-1-benzopyran-2-carbonyl]amino}-2-hydroxybicyclo[2.2.2]octan-1-yl]-4-methyl-1,3-thiazole-2-carboxamide (Compound 354)

The title compound was prepared using the methodologies described above. ¹H NMR (400 MHz, DMSO-d₆) δ ppm 7.76 (s, 1H), 7.66-7.58 (m, 3H), 7.56 (d, J=1.1 Hz, 1H), 7.16 (dd, J=8.6, 0.6 Hz, 1H), 5.33 (br s, 1H), 5.05 (dd, J=8.1, 5.0 Hz, 1H), 4.01-3.95 (m, 1H), 3.01-2.84 (m, 2H), 2.47-2.38 (m, 4H), 2.34-2.24 (m, 1H), 2.09-2.00 (m, 1H), 1.98-1.88 (m, 2H), 1.85-1.69 (m, 5H); MS (APCI⁺) m/z 490 (M+H)⁺.

### Example 256: (2R,4R)-6-chloro-N-[(2S)-4-{2-[(4-fluoro-1H-indazol-6-yl)oxy]acetamido}-2-hydroxybicyclo[2.2.2]octan-1-yl]-4-hydroxy-3,4-dihydro-2H-1-benzopyran-2-carboxamide (Compound 355)

### Example 256A: tert-butyl [(2S)-4-{2-[(4-fluoro-1H-indazol-6-yl)oxy]acetamido}-2-hydroxybicyclo[2.2.2]octan-1-yl]carbamate

The reaction and purification conditions described in Example 108E substituting the product of Example 85C for the product of Example 108A, and 2-((4-fluoro-1H-indazol-6-yl)oxy)acetic acid (prepared as described in International Patent Publication WO2017/193034, 2017, A1) for the product of Example 108D gave the title compound. MS (ESI⁻) m/z 447 (M−H)⁻

### Example 256B: (S)—N-(4-amino-3-hydroxybicyclo[2.2.2]octan-1-yl)-2-((4-fluoro-1H-indazol-6-yl)oxy)acetamide, bis-trifluoroacetate

The reaction and purification conditions described in Example 114C substituting the product of Example 256A for the product of Example 114B gave the title compound as a trifluoroacetic acid salt. MS (ESI⁺) m/z 349 (M+H)⁺.

### Example 256C: (2R,4R)-6-chloro-N-[(2S)-4-{2-[(4-fluoro-1H-indazol-6-yl)oxy]acetamido}-2-hydroxybicyclo[2.2.2]octan-1-yl]-4-hydroxy-3,4-dihydro-2H-1-benzopyran-2-carboxamide

The reaction and purification conditions described in Example 245D substituting the product of Example 256B for the product of Example 245B gave the title compound. ¹H NMR (400 MHz, DMSO-d₆) δ ppm 13.18 (s, 1H), 8.04 (s, 1H), 7.52 (s, 1H), 7.39-7.31 (m, 1H), 7.26-7.16 (m, 2H), 6.93-6.82 (m, 1H), 6.74-6.71 (m, 1H), 6.64 (dd, J=11.6, 1.8 Hz, 1H), 5.74-5.57 (m, 1H), 5.24-5.11 (m, 1H), 4.78 (t, J=8.4 Hz, 1H), 4.63-4.53 (m, 1H), 4.47 (s, 2H), 4.03 (d, J=9.2 Hz, 1H), 2.37-2.24 (m, 2H), 2.25-2.13 (m, 1H), 2.01-1.76 (m, 8H), 1.77-1.64 (m, 1H); MS (APCI⁺) m/z 559 (M+H)⁺.

### Example 257: N-[(2S)-4-{[(2R,4R)-6-chloro-4-hydroxy-3,4-dihydro-2H-1-benzopyran-2-carbonyl]amino}-2-hydroxybicyclo[2.2.2]octan-1-yl]-4-methyl-1,3-thiazole-2-carboxamide (Compound 356)

The reaction and purification conditions described in Example 108F substituting the product of Example 255 for the product of Example 108E gave the title compound. ¹H NMR (400 MHz, DMSO-d₆) δ ppm 7.64 (s, 1H), 7.59-7.54 (m, 1H), 7.41-7.35 (m, 2H), 7.18 (dd, J=8.7, 2.7 Hz, 1H), 6.87 (d, J=8.7 Hz, 1H), 5.67 (br s, 2H), 4.78 (dd, J=10.6, 5.9 Hz, 1H), 4.56 (dd, J=11.8, 2.2 Hz, 1H), 4.05-3.96 (m, 1H), 2.49-2.44 (m, 1H), 2.41 (d, J=0.9 Hz, 3H), 2.40-2.32 (m, 1H), 2.27 (ddd, J=12.8, 5.9, 2.3 Hz, 1H), 2.13-2.02 (m, 1H), 2.02-1.92 (m, 2H), 1.92-1.81 (m, 4H), 1.81-1.69 (m, 2H); MS (APCI⁺) m/z 492 (M+H)⁺.

### Example 258: N-(4-{[(2R)-6-chloro-4-oxo-3,4-dihydro-2H-1-benzopyran-2-carbonyl]amino}bicyclo[2.2.2]octan-1-yl)-5-(trifluoromethoxy)pyridine-2-carboxamide (Compound 357)

### Example 258A: N-(4-aminobicyclo[2.2.2]octan-1-yl)-5-(trifluoromethoxy)picolinamide, bis-trifluoroacetate

The reaction and purification conditions described in Example 108E substituting the product of Example 1A for the product of Example 108A, and 5-(trifluoromethoxy)picolinic acid (Enamine) for the product of Example 108D gave the tert-butyl (4-(5-(trifluoromethoxy)picolinamido)bicyclo[2.2.2]octan-1-yl)carbamate intermediate which was further processed with trifluoroacetic acid as described in Example 114C and then purified by preparative HPLC [YMC TriArt™ Hybrid ODS 5 μm column, 50×100 mm, flow rate 140 mL/minute, 5-100% gradient of acetonitrile in buffer (0.1% TFA)] to give the title compound as a trifluoroacetic acid salt. MS (APCI⁺) m/z 330 (M+H)⁺.

### Example 258B: N-(4-{[(2R)-6-chloro-4-oxo-3,4-dihydro-2H-1-benzopyran-2-carbonyl]amino}bicyclo[2.2.2]octan-1-yl)-5-(trifluoromethoxy)pyridine-2-carboxamide

The reaction and purification conditions described in Example 108E substituting the product of Example 258A for the product of Example 108A, and the product of Example 124A for the product of Example 108D gave the title compound. ¹H NMR (501 MHz, DMSO-d₆) δ ppm 8.69 (d, J=2.7 Hz, 1H), 8.16-8.09 (m, 1H), 8.09-8.02 (m, 1H), 7.89 (s, 1H), 7.73 (s, 1H), 7.66-7.58 (m, 2H), 7.16 (d, J=8.6 Hz, 1H), 5.06 (dd, J=8.1, 5.1 Hz, 1H), 3.01-2.87 (m, 2H), 2.09-1.87 (m, 12H); MS (APCI⁺) m/z 538 (M+H)⁺.

### Example 259: N-(4-{[(2S)-6-chloro-4-oxo-3,4-dihydro-2H-1-benzopyran-2-carbonyl]amino}bicyclo[2.2.2]octan-1-yl)-5-(trifluoromethoxy)pyridine-2-carboxamide (Compound 358)

The reaction and purification conditions described in Example 108E substituting the product of Example 258A for the product of Example 108A, and the product of Example 125A for the product of Example 108D gave the title compound. ¹H NMR (400 MHz, DMSO-d₆) δ ppm 8.69 (d, J=2.6 Hz, 1H), 8.17-8.09 (m, 1H), 8.09-8.02 (m, 1H), 7.89 (s, 1H), 7.73 (s, 1H), 7.66-7.58 (m, 2H), 7.16 (d, J=8.6 Hz, 1H), 5.06 (dd, J=8.1, 5.1 Hz, 1H), 3.01-2.85 (m, 2H), 2.07-1.87 (m, 12H); MS (APCI⁺) m/z 538 (M+H)⁺.

### Example 260: (2R,4R)-6,7-difluoro-N-{3-[2-(4-fluoro-3-methylphenoxy)acetamido]bicyclo[1.1.1]pentan-1-yl}-4-hydroxy-3,4-dihydro-2H-1-benzopyran-2-carboxamide (Compound 359)

### Example 260A: 2-(4-fluoro-3-methylphenoxy)acetic Acid

The reaction and purification conditions described in Example 128C substituting 4-fluoro-3-methylphenol for 2-methoxypyrimidin-5-ol gave the title compound. ¹H NMR (400 MHz, DMSO-d₆) δ ppm 7.03 (t, J=9.2 Hz, 1H), 6.84 (dd, J=6.4, 3.2 Hz, 1H), 6.72 (dt, J=8.9, 3.6 Hz, 1H), 4.62 (s, 2H), 2.19 (d, J=2.0 Hz, 3H).

### Example 260B: N-(3-aminobicyclo[1.1.1]pentan-1-yl)-2-(4-fluoro-3-methylphenoxy)acetamide, bis-trifluoroacetate

The reaction and purification conditions described in Example 108E substituting tert-butyl (3-aminobicyclo[1.1.1]pentan-1-yl)carbamate for the product of Example 108A, and the product of Example 260A for the product of Example 108D gave the tert-butyl (3-(2-(4-fluoro-3-methylphenoxy)acetamido)bicyclo[1.1.1]pentan-1-yl)carbamate intermediate which was further processed with trifluoroacetic acid as described in Example 114C to give the title compound. MS (APCI⁺) m/z 265 (M+H)⁺.

### Example 260C: (2R,4R)-6,7-difluoro-N-{3-[2-(4-fluoro-3-methylphenoxy)acetamido]bicyclo[1.1.1]pentan-1-yl}-4-hydroxy-3,4-dihydro-2H-1-benzopyran-2-carboxamide

The reaction and purification conditions described in Example 108E through Example 108F substituting the product of Example 260B for the product of Example 108A gave the title compound. ¹H NMR (400 MHz, DMSO-d₆) δ ppm 8.66 (s, 1H), 8.66 (s, 1H), 7.33 (ddd, J=11.4, 9.3, 1.0 Hz, 1H), 7.05 (t, J=9.2 Hz, 1H), 6.95-6.87 (m, 2H), 6.78 (dt, J=9.0, 3.6 Hz, 1H), 5.71 (d, J=6.1 Hz, 1H), 4.77 (dt, J=11.5, 6.0 Hz, 1H), 4.62 (dd, J=11.9, 2.3 Hz, 1H), 4.38 (s, 2H), 2.35 (ddd, J=13.0, 5.9, 2.4 Hz, 1H), 2.28 (s, 6H), 2.20 (d, J=2.0 Hz, 3H), 1.69 (ddd, J=13.0, 12.1, 10.7 Hz, 1H); MS (APCI⁺) m/z 459 (M+H)⁺.

### Example 261: (2S,4S)-6-chloro-N-{3-[2-(4-fluoro-3-methylphenoxy)acetamido]bicyclo[1.1.1]pentan-1-yl}-4-hydroxy-3,4-dihydro-2H-1-benzopyran-2-carboxamide (Compound 360)

### Example 261A: (2S,4S)-6-chloro-4-hydroxychroman-2-carboxylic Acid

The reaction and purification conditions described in Example 245C substituting the product of Example 125A for the product of Example 124A gave the title compound. MS (APCI⁻) m/z 227 (M−H)⁻.

### Example 261B: (2S,4S)-6-chloro-N-{3-[2-(4-fluoro-3-methylphenoxy)acetamido]bicyclo[1.1.1]pentan-1-yl}-4-hydroxy-3,4-dihydro-2H-1-benzopyran-2-carboxamide

The reaction and purification conditions described in Example 108E substituting the product of Example 261A for the product of Example 108D, and the product of Example 260B for the product of Example 108A gave the title compound. ¹H NMR (400 MHz, DMSO-d₆) δ ppm 8.67 (s, 1H), 8.65 (s, 1H), 7.40-7.35 (m, 1H), 7.20 (dd, J=8.7, 2.7 Hz, 1H), 7.05 (t, J=9.2 Hz, 1H), 6.91-6.86 (m, 2H), 6.77 (dt, J=8.9, 3.6 Hz, 1H), 5.69 (d, J=6.3 Hz, 1H), 4.80 (dt, J=11.8, 6.1 Hz, 1H), 4.59 (dd, J=11.9, 2.3 Hz, 1H), 4.38 (s, 2H), 2.35 (ddd, J=12.8, 5.8, 2.3 Hz, 1H), 2.28 (s, 6H), 2.20 (d, J=2.0 Hz, 3H), 1.69 (td, J=12.4, 10.7 Hz, 1H); MS (APCI⁺) m/z 459 (M+H)⁺.

### Example 262: N-(4-{[(2R,4R)-6-chloro-4-hydroxy-3,4-dihydro-2H-1-benzopyran-2-carbonyl]amino}bicyclo[2.2.2]octan-1-yl)-5-(trifluoromethoxy)pyridine-2-carboxamide (Compound 361)

The reaction and purification conditions described in Example 108F substituting the product of Example 258 for the product of Example 108E gave the title compound. ¹H NMR (400 MHz, DMSO-d₆) δ ppm 8.73-8.67 (m, 1H), 8.13 (dd, J=8.7, 0.7 Hz, 1H), 8.10-8.03 (m, 1H), 7.90 (s, 1H), 7.38 (dd, J=2.7, 1.0 Hz, 1H), 7.35 (s, 1H), 7.20-7.14 (m, 1H), 6.87 (d, J=8.7 Hz, 1H), 5.66 (d, J=6.3 Hz, 1H), 4.78 (dt, J=11.4, 6.1 Hz, 1H), 4.57 (dd, J=11.8, 2.2 Hz, 1H), 2.28 (ddd, J=12.9, 6.0, 2.3 Hz, 1H), 2.10-1.94 (m, 12H), 1.74 (ddd, J=13.0, 11.7, 10.7 Hz, 1H); MS (APC⁺) m/z 522 (M+H)⁺.

### Example 263: N-(4-{[(2S,4S)-6-chloro-4-hydroxy-3,4-dihydro-2H-1-benzopyran-2-carbonyl]amino}bicyclo[2.2.2]octan-1-yl)-5-(trifluoromethoxy)pyridine-2-carboxamide (Compound 362)

The reaction and purification conditions described in Example 108F substituting the product of Example 259 for the product of Example 108E gave the title compound. ¹H NMR (500 MHz, DMSO-d₆) δ ppm 8.72-8.68 (m, 1H), 8.13 (dd, J=8.7, 0.7 Hz, 1H), 8.10-8.04 (m, 1H), 7.91 (s, 1H), 7.38 (dd, J=2.7, 1.0 Hz, 1H), 7.36 (s, 1H), 7.18 (ddd, J=8.7, 2.7, 0.7 Hz, 1H), 6.87 (d, J=8.7 Hz, 1H), 5.68 (d, J=6.3 Hz, 1H), 4.78 (dt, J=11.4, 6.0 Hz, 1H), 4.57 (dd, J=11.8, 2.2 Hz, 1H), 2.28 (ddd, J=12.9, 6.0, 2.3 Hz, 1H), 2.08-1.93 (m, 12H), 1.74 (ddd, J=12.9, 11.9, 10.8 Hz, 1H); MS (APC⁺) m/z 522 (M+H)⁺.

### Example 264 6-chloro-N-{4-[2-(4-chloro-3-fluorophenoxy)acetamido]-3-oxo-2-azabicyclo[2.2.2]octan-1-yl}-4-oxo-3,4-dihydro-2H-1-benzopyran-2-carboxamide (Compound 363)

### Example 264A: methyl 8-amino-1,4-dioxaspiro[4.5]decane-8-carboxylate

To a suspension of 8-amino-1,4-dioxaspiro[4.5]decane-8-carboxylic acid (2.62 g, 13.02 mmol, ArkPharm) in methanol (7.5 mL) and dichloromethane (30 mL), trimethylsilyldiazomethane (9.77 mL, 19.53 mmol) was added at 0° C., and the mixture was stirred at ambient temperature overnight. The reaction was quenched with acetic acid (2.0 mL), and the mixture was stirred for 5 minutes. The reaction mixture was partitioned between saturated NaHCO₃and dichloromethane. The organic layer was dried over magnesium sulfate and filtered. The filtrate was concentrated to give 2.97 g of the title compound, which was used without further purification. ¹H NMR (400 MHz, DMSO-d₆) δ ppm 3.78 (d, J=0.8 Hz, 4H), 3.56 (s, 3H), 1.88-1.75 (m, 2H), 1.75 (d, J=3.9 Hz, 2H), 1.76-1.64 (m, 2H), 1.53-1.42 (m, 2H), 1.44-1.34 (m, 2H); MS (ESI⁺) m/z 216.1 (M+H)⁺.

### Example 264B: methyl 8-(2-(4-chloro-3-fluorophenoxy)acetamido)-1,4-dioxaspiro[4.5]decane-8-carboxylate

A mixture of Example 264A (0.4 g, 1.86 mmol), 2-(3,4-dichlorophenoxy)acetic acid (0.48 g, 2.32 mmol) and N-ethyl-N-isopropylpropan-2-amine (1.14 mL, 6.50 mmol) in N,N-dimethylformamide (10.0 mL) was treated with 2-(3H-[1,2,3]triazolo[4,5-b]pyridin-3-yl)-1,1,3,3-tetramethylisouronium hexafluorophosphate (V) (1.06 g, 2.79 mmol), and the reaction mixture was stirred at ambient temperature for about 16 hours. The mixture was partitioned between water and dichloromethane. The organic layer was washed with brine, dried over magnesium sulfate and filtered. The filtrate was concentrated and purified by HPLC (Phenomenex® Luna® C18(2) 10 μm 100 Å AXIA™ column (250 mm×50 mm). A 25-95% gradient of acetonitrile (A) and 0.1% trifluoroacetic acid in water (B) was used over 25 minutes, at a flow rate of 50 mL/minute) to give 0.46 g of the title compound. ¹H NMR (400 MHz, DMSO-d₆) δ ppm 8.36 (s, 1H), 7.49 (t, J=8.9 Hz, 1H), 6.99 (dd, J=11.4, 2.9 Hz, 1H), 6.81 (ddd, J=8.9, 2.9, 1.2 Hz, 1H), 4.61 (s, 2H), 3.86 (s, 4H), 3.55 (s, 3H), 2.04 (d, J=13.7 Hz, 2H), 1.91 (td, J=13.3, 12.9, 4.4 Hz, 2H), 1.68-1.53 (m, 4H); MS (ESI⁺) m/z 402.2 (M+H)⁺.

### Example 264C: methyl 1-(2-(4-chloro-3-fluorophenoxy)acetamido)-4-oxocyclohexanecarboxylate

To a solution of Example 264B (3.3 g, 8.21 mmol) in acetone (30 mL), 3 N HCl (30 mL, 90 mmol) was added, and the reaction mixture was stirred for 3 hours at ambient temperature to give a suspension. The precipitate was collected by filtration, washed with water, and dried under vacuum at 50° C. to give 2.82 g of the title compound which was used without further purification. ¹H NMR (400 MHz, DMSO-d₆) δ ppm 8.63 (s, 1H), 7.50 (t, J=8.9 Hz, 1H), 7.02 (dd, J=11.4, 2.9 Hz, 1H), 6.83 (ddd, J=9.0, 2.9, 1.2 Hz, 1H), 4.66 (s, 2H), 3.59 (s, 3H), 2.44 (dq, J=16.1, 5.1 Hz, 2H), 2.37-2.27 (m, 2H), 2.26-2.06 (m, 4H); MS (ESI⁺) m/z 358.2 (M+H)⁺.

### Example 264D: N-(1-amino-3-oxo-2-azabicyclo[2.2.2]octan-4-yl)-2-(4-chloro-3-fluorophenoxy)acetamide

A mixture of Example 264C (2.82 g, 7.88 mmol) and 7 N ammonia (50 mL, 350 mmol) in methanol (50 mL) was stirred at ambient temperature for 24 hours. The precipitate in the suspension was collected by filtration and air-dried to give 1.99 g of the title compound. ¹H NMR (400 MHz, DMSO-d₆) δ ppm 8.12 (s, 1H), 7.82 (s, 1H), 7.51 (t, J=8.8 Hz, 1H), 7.11 (dd, J=11.3, 2.9 Hz, 1H), 6.86 (ddd, J=8.9, 2.9, 1.2 Hz, 1H), 4.57 (s, 2H), 2.66 (ddd, J=12.5, 10.7, 4.2 Hz, 2H), 2.27 (s, 2H), 1.77 (td, J=11.5, 4.3 Hz, 2H), 1.60 (td, J=11.2, 4.4 Hz, 2H), 1.42 (ddt, J=14.8, 12.9, 3.2 Hz, 2H); MS (ESI⁺) m/z 342.3 (M+H)⁺.

### Example 264E: 6-chloro-N-{4-[2-(4-chloro-3-fluorophenoxy)acetamido]-3-oxo-2-azabicyclo[2.2.2]octan-1-yl}-4-oxo-3,4-dihydro-2H-1-benzopyran-2-carboxamide

To a mixture of Example 264D (75 mg, 0.219 mmol), 6-chloro-4-oxochroman-2-carboxylic acid (52.2 mg, 0.230 mmol), and N-ethyl-N-isopropylpropan-2-amine (0.153 mL, 0.878 mmol) in N,N-dimethylformamide (2.0 mL), 2-(3H-[1,2,3]triazolo[4,5-b]pyridin-3-yl)-1,1,3,3-tetramethylisouronium hexafluorophosphate (V) (104 mg, 0.274 mmol) was added, and the mixture was stirred at ambient temperature for 30 minutes. Volatiles were removed, and the residue was purified by HPLC (Phenomenex® Luna® C18(2) 10 μm 100 Å AXIA™ column (250 mm×50 mm). A 30-100% gradient of acetonitrile (A) and 0.1% trifluoroacetic acid in water (B) was used over 25 minutes, at a flow rate of 50 mL/minute) to give 76 mg of the title compound. ¹H NMR (400 MHz, DMSO-d₆) δ ppm 8.78 (s, 1H), 8.57 (s, 1H), 7.83 (s, 1H), 7.68 7.59 (m, 2H), 7.50 (t, J=8.8 Hz, 1H), 7.20-7.07 (m, 2H), 6.87 (ddd, J=9.0, 2.9, 1.2 Hz, 1H), 5.11 (t, J=7.0 Hz, 1H), 4.58 (s, 2H), 3.00 (d, J=7.0 Hz, 2H), 2.70 (td, J=11.2, 5.6 Hz, 2H), 2.34 (td, J=11.9, 5.7 Hz, 1H), 2.20-2.05 (m, 1H), 1.89 (ddt, J=18.4, 12.2, 5.8 Hz, 2H), 1.51 (tq, J=11.2, 3.8, 3.2 Hz, 2H); MS (APCI⁺) m/z 549.95 (M+H)⁺.

### Example 265 rac-(2R,4R)-6-chloro-N-{4-[2-(4-chloro-3-fluorophenoxy)acetamido]-3-oxo-2-azabicyclo[2.2.2]octan-1-yl}-4-hydroxy-3,4-dihydro-2H-1-benzopyran-2-carboxamide (Compound 364)

To a suspension of Example 264 (0.084 g, 0.153 mmol) in methanol (2.0 mL), sodium tetrahydroborate (0.012 g, 0.305 mmol) was added, and the reaction mixture was stirred at ambient temperature for 45 minutes. Water and saturated ammonium chloride solution (4 mL, 1:1) were added. The precipitate was collected by filtration, washed with water and heptane, and air-dried to give 74 mg of the title compound. ¹H NMR (400 MHz, DMSO-d₆) δ ppm 8.75 (s, 1H), 8.22 (s, 1H), 7.85 (s, 1H), 7.51 (t, J=8.8 Hz, 1H), 7.38 (d, J=2.7 Hz, 1H), 7.20 (dd, J=8.7, 2.7 Hz, 1H), 7.12 (dd, J=11.3, 2.9 Hz, 1H), 6.92-6.83 (m, 2H), 5.72 (d, J=6.2 Hz, 1H), 4.80 (dt, J=11.5, 6.0 Hz, 1H), 4.65 (dd, J=11.5, 2.4 Hz, 1H), 4.59 (s, 2H), 2.71 (td, J=11.9, 4.2 Hz, 2H), 2.36 (ddd, J=13.2, 5.9, 2.4 Hz, 1H), 2.32-2.17 (m, 1H), 1.96 (dd, J=13.5, 9.7 Hz, 2H), 1.84 (q, J=11.8 Hz, 1H), 1.58-1.47 (m, 2H); MS (APCI⁺) m/z 551.99 (M+H)⁺.

### Example 266 6-chloro-4-oxo-N-{rac-(1R,2S,4R,5S)-5-[4-(trifluoromethyl)benzamido]bicyclo[2.2.1]heptan-2-yl}-3,4-dihydro-2H-1-benzopyran-2-carboxamide (Compound 365)

### Example 266A: N-[rac-(1R,2S,4R)-bicyclo[2.2.1]hept-5-en-2-yl]-4-(trifluoromethyl)benzamide

A 20 mL screw-capped vial was charged with chlorobis(cyclooctene)iridium(i)dimer ([Ir(coe)₂Cl]₂, 0.190 g, 0.211 mmol), (S)-(+)-5,5′-bis[di(3,5-di-tert-butyl-4-methoxyphenyl)phosphino]-4,4′-bi-1,3-benzodioxole (Aldrich, 0.50 g, 0.42 mmol) and 4-(trifluoromethyl)benzamide (2.0 g, 10.6 mmol). Toluene (10.6 mL) was added to these solids, and the mixture was stirred vigorously at 50° C. for 30 minutes. 2,5-Norbornadiene (4.30 mL, 42.3 mmol) was added to the resultant suspension. The vial was sealed with a polytetrafluoroethylene cap and stirred at 125° C. for 15 hours. The reaction mixture was then taken up in N,N-dimethylformamide (3 mL) and concentrated under reduced pressure to remove most of the toluene. The residue was then diluted again in N,N-dimethylformamide (10 mL), filtered through a glass microfiber frit and purified by preparative HPLC [YMC TriArt™ C18 Hybrid 5 μm column, 50×100 mm, flow rate 140 mL/minute, 20-100% gradient of acetonitrile in buffer (0.025 M aqueous ammonium bicarbonate, adjusted to pH 10 with ammonium hydroxide)] to give the title compound (0.99 g, 3.52 mmol, 33% yield). MS (APCI⁺) m/z 282 (M+H)⁺.

### Example 266B: N,N′-[rac-(1R,2S,4R,5S)-bicyclo[2.2.1]heptane-2,5-diyl]bis[4-(trifluoromethyl)benzamide]

A 20 mL screw-capped vial was charged with chlorobis(cyclooctene)iridium(i)dimer ([Ir(coe)₂Cl]₂, 147 mg, 0.164 mmol), (S)-(+)-5,5′-bis[di(3,5-di-tert-butyl-4-methoxyphenyl)phosphino]-4,4′-bi-1,3-benzodioxole (Aldrich, 386 mg, 0.33 mmol), and 4-(trifluoromethyl)benzamide (1.08 g, 5.72 mmol). Toluene (1.6 mL) was added to these solids, and the mixture was stirred vigorously at 50° C. for 30 minutes. The product of Example 264A (0.46 g, 1.64 mmol) was added to the resultant suspension. The vial was sealed with a polytetrafluoroethylene cap and stirred at 125° C. for 15 hours. The reaction mixture was then taken up in N,N-dimethylformamide (3 mL) and concentrated under reduced pressure to remove most of the toluene. The residue was then diluted again in N,N-dimethylformamide (10 mL), filtered through a glass microfiber frit and purified by preparative HPLC [YMC TriArt™ C18 Hybrid 5 μm column, 50×100 mm, flow rate 140 mL/minute, 20-100% gradient of acetonitrile in buffer (0.025 M aqueous ammonium bicarbonate, adjusted to pH 10 with ammonium hydroxide)] to give the title compound (0.15 g, 0.32 mmol, 20% yield). ¹H NMR (400 MHz, DMSO-d₆) δ ppm 8.40 (d, J=6.6 Hz, 2H), 8.07-7.98 (m, 4H), 7.86-7.79 (m, 4H), 3.79-3.70 (m, 2H), 2.26 (d, J=4.6 Hz, 2H), 1.77-1.67 (m, 2H), 1.63-1.53 (m, 4H); MS (APCI⁺) m/z 471 (M+H)⁺.

### Example 266C: N-[rac-(1R,2S,4R,5S)-5-aminobicyclo[2.2.1]heptan-2-yl]-4-(trifluoromethyl)benzamide

A 20 mL microwave vial was charged with the product of Example 266B (45 mg, 0.096 mmol), acetic acid (2 mL) and aqueous HCl (12 N, 2 mL). The vial was sealed and stirred for 1 hour at 165° C. in a Biotage® Initiator microwave reactor. The resulting mixture was then concentrated under reduced pressure to dryness, and the residue was purified by preparative HPLC [YMC TriArt™ C18 Hybrid 5 μm column, 50×100 mm, flow rate 140 mL/minute, 5-100% gradient of acetonitrile in buffer (0.025 M aqueous ammonium bicarbonate, adjusted to pH 10 with ammonium hydroxide)] to give the title compound (2.5 mg, 8.4 μmol, 9% yield). MS (APCI⁺) m/z 299 (M+H)⁺.

### Example 266D: 6-chloro-4-oxochroman-2-carbonyl chloride

Dichloromethane (2 mL) was added to 6-chloro-4-oxochroman-2-carboxylic acid (Princeton Bio, 50 mg, 0.22 mmol) and stirred at ambient temperature. One drop of N,N-dimethylformamide was added followed by oxalyl chloride solution (2.0 M in dichloromethane, 0.22 mL). The resulting mixture was stirred for 30 minutes and then concentrated under high vacuum to give the title compound (55 mg, 0.22 mmol, 100% yield) which was used as is without further purification.

### Example 266E: 6-chloro-4-oxo-N-{rac-(1R,2S,4R,5S)-5-[4-(trifluoromethyl)benzamido]bicyclo[2.2.1]heptan-2-yl}-3,4-dihydro-2H-1-benzopyran-2-carboxamide

The product of Example 266C (2.5 mg, 8.4 μmol), dichloromethane (1 mL) and pyridine (0.1 mL) were combined and stirred at ambient temperature. The product of Example 266D (6.2 mg, 0.025 mmol) was added in one portion. The resulting mixture was stirred for 30 minutes and then concentrated under reduced pressure. The residue was directly purified by preparative HPLC [Waters XBridge™ C18 5 μm OBD column, 30×100 mm, flow rate 40 mL/minute, 5-100% gradient of acetonitrile in buffer (0.025 M aqueous ammonium bicarbonate, adjusted to pH 10 with ammonium hydroxide)] to give the title compound (3.8 mg, 7.5 μmol, 89% yield). ¹H NMR (400 MHz, DMSO-d₆) δ ppm 8.38 (t, J=6.9 Hz, 2H), 8.20-8.14 (m, 1H), 8.02 (d, J=8.0 Hz, 2H), 7.83 (d, J=8.1 Hz, 2H), 7.68-7.59 (m, 2H), 7.18 (dd, J=8.6, 3.5 Hz, 1H), 5.15-5.07 (m, 1H), 3.01-2.94 (m, 2H), 2.24-2.20 (m, 2H), 2.16-1.98 (m, 1H), 1.70-1.59 (m, 2H), 1.52 (t, J=14.9 Hz, 2H), 1.38 (dt, J=28.5, 9.3 Hz, 2H); MS (APCI⁺) m/z 507 (M+H)⁺.

### Example 267: (2RS,4RS)-6-chloro-4-hydroxy-N-{(1SR,2RS,4SR,5RS)-5-[4-(trifluoromethyl)benzamido]bicyclo[2.2.1]heptan-2-yl}-3,4-dihydro-2H-1-benzopyran-2-carboxamide (Compound 366)

The reaction and purification conditions described in Example 108F substituting the product of Example 266E for the product of Example 108E gave the title compound. ¹H NMR (400 MHz, methanol-d₄) δ ppm 7.95 (d, J=8.0 Hz, 2H), 7.76 (d, J=8.2 Hz, 2H), 7.42 (dd, J=2.7, 0.9 Hz, 1H), 7.15 (dd, J=8.7, 2.6 Hz, 1H), 6.91 (d, J=8.8 Hz, 1H), 4.95-4.88 (m, 1H), 4.64 (dt, J=11.4, 2.2 Hz, 1H), 3.82 (dd, J=8.3, 3.6 Hz, 1H), 3.75-3.68 (m, 1H), 2.51 (ddt, J=13.1, 6.0, 2.0 Hz, 1H), 2.42-2.37 (m, 1H), 2.33 (dd, J=9.7, 4.7 Hz, 1H), 1.98-1.81 (m, 3H), 1.64-1.47 (m, 4H); MS (APCI⁺) m/z 491 (M−H₂O+H)⁺.

### Example 268: (2R,4S)-6-chloro-N-{3-[2-(4-chloro-3-fluorophenoxy)acetamido]bicyclo[1.1.1]pentan-1-yl}-7-fluoro-4-hydroxy-3,4-dihydro-2H-1-benzopyran-2-carboxamide (Compound 367)

Example 165 (0.025 g, 0.049 mmol) was dissolved in trifluoroacetic acid (8.5 mL, 111 mmol) and stirred at 35° C. for 1 hour. Then the solution was concentrated in vacuo. The resulting residue was dissolved in acetonitrile (2 mL) and then 5% ammonium hydroxide aqueous buffer (2 mL) was added and the mixture was stirred at ambient temperature for 10 minutes before being concentrated in vacuo. The crude residue was purified by preparative HPLC (Phenomenex® Luna® C8(2) 5 μm AXIA™ column (150 mm×30 mm). A 30-100% gradient of acetonitrile (A) and 0.1% trifluoroacetic acid in water (B) was used over 25 minutes, at a flow rate of 50 mL/minute) to give the title compound as a mixture with starting material Example 165 (0.018 g, dr 5:1, trans:cis). This mixture of diastereomers was further purified by chiral SFC (supercritical fluid chromatography) using a Chiralpak® IB, 250×21 mm I.D., 5 μm column eluting with 20% CH₃OH in CO₂ with a flow rate of 80 g/minute and back pressure of 120 bar to give the title compound (first isomer eluted, 0.011 g, 0.021 mmol, 73% yield). ¹H NMR (400 MHz, DMSO-d₆) δ ppm 8.77 (s, 1H), 8.73 (s, 1H), 7.50 (t, J=8.9 Hz, 1H), 7.46 (d, J=8.5 Hz, 1H), 7.07 (dd, J=11.2, 2.7 Hz, 1H), 6.97 (d, J=10.6 Hz, 1H), 6.85 (d, J=9.4 Hz, 1H), 4.58 (dd, J=7.4, 3.4 Hz, 2H), 4.48 (s, 2H), 2.28 (s, 6H), 2.09 (d, J=13.6 Hz, 1H), 1.96-1.85 (m, 1H); MS (APCI⁺) m/z 513 (M+H)⁺.

### Example 269: (2R)-6-chloro-N-{(1RS,2SR,4RS,5SR)-5-[2-(4-chloro-3-fluorophenoxy)acetamido]bicyclo[2.2.1]heptan-2-yl}-4-oxo-3,4-dihydro-2H-1-benzopyran-2-carboxamide (Compound 368)

### Example 269A: tetramethyl 2,6-dioxobicyclo[3.3.1]nonane-1,3,5,7-tetracarboxylate

To a solution of dimethyl malonate (264 g, 1998 mmol) in toluene (400 mL) stirred at 20° C. was added formaldehyde (37 weight % in water, 45.0 g) and piperidine (4 mL, 40.4 mmol) in sequential order. The reaction was refluxed at 110° C. in a Dean-Stark trap. The refluxing was continued until water ceased to accumulate in the trap. The reaction mixture was cooled and most of the solvent was removed under reduced pressure. The residue was taken up in absolute methanol (150 mL) and added rapidly to a stirring solution of sodium (22.97 g, 999 mmol) in methanol (400 mL). More methanol was added as needed to facilitate stirring. The mixture was stirred and refluxed gently at 70° C. for 7 hours, cooled, and then concentrated under reduced pressure. The resulting residue was taken up in ice water (200 mL) and washed several times with tert-butyl methyl ether. Precipitation of the product was achieved by bubbling carbon dioxide gas through the aqueous layer. The precipitate was collected by vacuum filtration, and the filter cake was washed with water and then air dried. Residual water was removed by azeotropic distillation with benzene, the benzene solution was filtered while hot and then cooled. The crystallized solids were isolated by suction filtration and dried at 20° C. under reduced pressure to give the title compound (25 g, 65 mmol, 6.5% yield). ¹H NMR (400 MHz, CDCl₃) δ ppm 2.22-2.42 (m, 1H) 2.61-2.73 (m, 2H) 2.88 (s, 1H) 3.04-3.12 (m, 1H) 3.52-3.60 (m, 1H) 3.73-3.83 (m, 12H) 12.17 (s, 1H).

### Example 269B: bicyclo[3.3.1]nonane-2,6-dione

To a mixture of the product of Example 269A (25 g, 65 mmol) in acetic acid (90 mL) stirred at 20° C. was added water (30 mL) and HCl (11.6 M, 30 mL) in sequential order. The resulting mixture was stirred at 100° C. for 12 hours and then concentrated under reduced pressure in a 50° C. bath. The resulting residue was purified by flash chromatography (SiO₂, 50% petroleum ether in ethyl acetate) to give the title compound (2 g, 11.8 mmol, 18% yield). ¹H NMR (400 MHz, CDCl₃) δ ppm 1.98-2.15 (m, 4H) 2.22 (br s, 2H) 2.33-2.48 (m, 2H) 2.55-2.67 (m, 2H) 2.76 (br s, 2H).

### Example 269C: dimethyl rac-(R,2S,4R,5S)-bicyclo[2.2.1]heptane-2,5-dicarboxylate

To a mixture of the product of Example 269B (3 g, 17.7 mmol) in methanol (30 mL) stirred at 20° C. was added a solution of thallium (III) nitrate trihydrate (23.65 g, 53.2 mmol) in methanol (30 mL). The mixture was stirred at 20° C. for 12 hours and then filtered. The filtrate was concentrated under reduced pressure. The residue was diluted with water (10 mL) and washed with ethyl acetate (20 mL). The organic phase was concentrated under reduced pressure to give the title compound (1.3 g, 4.90 mmol, 28% yield). ¹H NMR (400 MHz, CDCl₃) δ ppm 1.44 (s, 2H) 1.51-1.62 (m, 2H) 1.86-1.98 (m, 2H) 2.36 (dd, J=8.88, 5.50 Hz, 2H) 2.58 (br d, J=4.25 Hz, 2H) 3.68 (s, 6H).

### Example 269D: rac-(1R,2S,4R,5S)-5-(methoxycarbonyl)bicyclo[2.2.1]heptane-2-carboxylic Acid

To a mixture of the product of Example 269C (0.6 g, 2.26 mmol) in tetrahydrofuran (5 mL) stirred at 20° C. was added a solution of lithium hydroxide monohydrate (95 mg, 2.26 mmol) in water (2.0 mL). After stirring for 2 hours, the reaction solution was washed with ethyl acetate (5 mL). The aqueous layer was further diluted with water (10 mL), adjust the pH to 3 with aqueous HCl, and then extracted with ethyl acetate (2×20 mL). The ethyl acetate layers were combined and dried over sodium sulfate and concentrated under reduced pressure to give the title compound (0.3 g, 1.21 mmol, 53% yield). ¹H NMR (400 MHz, CDCl₃) δ ppm 1.46 (br d, J=9.6 Hz, 2H) 1.52-1.66 (m, 2H) 1.89-2.00 (m, 2H) 2.31-2.46 (m, 3H) 2.56-2.69 (m, 2H) 3.68 (d, J=2.3 Hz, 3H).

### Example 269E: methyl rac-(R,2S,4R,5S)-5-(((benzyloxy)carbonyl)amino)bicyclo[2.2.1]heptane-2-carboxylate

To a mixture of the product of Example 269D (1.1 g, 4.44 mmol) in toluene (10 mL) were added triethylamine (0.93 mL, 6.66 mmol) and diphenyl phosphorazidate (1.59 g, 5.77 mmol) in sequential order. The resulting mixture was stirred at 90° C. for 3 hours under nitrogen protection. The reaction mixture was cooled and concentrated under reduced pressure at 45° C. The resulting solution was quenched with benzyl alcohol (0.720 g, 6.66 mmol) and then stirred at 90° C. for 12 hours. The mixture was cooled and then concentrated under reduced pressure at 45° C. The residue was purified by flash chromatography (SiO₂, 9-10% ethyl acetate in petroleum ether) to give the title compound (0.8 g, 2.1 mmol, 33% yield). MS (ESI⁺) m/z 304 (M+H)⁺.

### Example 269F: rac-(1S,2R,4S,5R)-5-(((benzyloxy)carbonyl)amino)bicyclo[2.2.1]heptane-2-carboxylic acid

The reaction and purification conditions described in Example 269C substituting the product of Example 269E for the product of 269B, and also increasing the amount of lithium hydroxide monohydrate to 3 equivalents gave the title compound. MS (APCI⁺) m/z 290 (M+H)⁺.

### Example 269G: benzyl tert-butyl rac-((R,2S,4R,5S)-bicyclo[2.2.1]heptane-2,5-diyl)dicarbamate

To a solution of the product of Example 269F (0.15 g, 0.42 mmol) in tert-butanol (20 mL) were added triethylamine (0.087 mL, 0.62 mmol) and diphenyl phosphoryl azide (0.148 g, 0.54 mmol) in sequential order. The resulting mixture was stirred at 90° C. for 12 hours under nitrogen protection. The reaction mixture was cooled and concentrated under reduced pressure in a 45° C. bath. The residue was purified by silica gel column chromatography (20% ethyl acetate in petroleum ether) to give the title compound (200 mg (about 15% purity), 0.08 mmol, 9% yield). MS (ESI⁺) m/z 305 (M−C(CH₃)₃+H)⁺.

### Example 269H: tert-butyl (rac-(1R,2S,4R,5S)-5-aminobicyclo[2.2.1]heptan-2-yl)carbamate, monoformate

A mixture of palladium hydroxide (300 mg) and the product of Example 269G (300 mg (about 15% purity), 0.125 mmol) in tetrahydrofuran was stirred under hydrogen atmosphere (15 psi) for 12 hours. The reaction mixture was filtered and concentrated under reduced pressure. The residue was purified by preparative HPLC [Waters XBridge™ C18 10 μm OBD column, 30×100 mm, flow rate 40 mL/minute, 5-100% gradient of acetonitrile in buffer (0.2% formic acid)] to give the title compound (23 mg, 0.08 mmol, 58% yield). ¹H NMR (400 MHz, DMSO-d₆) δ ppm 1.14-1.30 (m, 3H) 1.34-1.39 (m, 9H) 1.47-1.58 (m, 3H) 2.01-2.12 (m, 2H) 2.86 (br d, J=4.52 Hz, 1H) 3.19-3.22 (m, 1H) 6.72-6.89 (m, 1H) 8.40 (s, 1H); MS (ESI⁺) m/z 227 (M+H)⁺.

### Example 269I: tert-butyl [(1RS,2SR,4RS,5SR)-5-{[(2R)-6-chloro-4-oxo-3,4-dihydro-2H-1-benzopyran-2-carbonyl]amino}bicyclo[2.2.1]heptan-2-yl]carbamate

The reaction and purification conditions described in Example 23C substituting the product of Example 296H for the product of Example 23B, the product of Example 124A for 2,3-dihydrobenzo[b][1,4]dioxine-2-carboxylic acid, and triethylamine for N-ethyl-N-isopropylpropan-2-amine gave the title compound. MS (APCI⁺) m/z 379 (M-tBu+H)⁺.

### Example 269J: (2R)-6-chloro-N-{(1RS,2SR,4RS,5SR)-5-[2-(4-chloro-3-fluorophenoxy)acetamido]bicyclo[2.2.1]heptan-2-yl}-4-oxo-3,4-dihydro-2H-1-benzopyran-2-carboxamide

Trifluoroacetic acid (100 μL, 1.3 mmol) was added to the product of Example 2691 (12 mg, 0.03 mmol) and stirred at ambient temperature for 30 minutes. The mixture was concentrated under reduced pressure, and N,N-dimethylformamide (1 mL), triethylamine (27 μL, 0.19 mmol), 2-(4-chloro-3-fluorophenoxy)acetic acid (5.6 mg, 0.03 mmol) and 1-[bis(dimethylamino)methylene]-1H-1,2,3-triazolo[4,5-b]pyridinium 3-oxid hexafluorophosphate (13.6 mg, 0.036 mmol) were added sequentially. The mixture was stirred for 1 hour, and water (0.1 mL) was added. The resulting solution was directly purified by preparative HPLC [Waters XBridge™ C18 5 μm OBD column, 30×100 mm, flow rate 40 mL/minute, 5-100% gradient of acetonitrile in buffer (0.025 M aqueous ammonium bicarbonate, adjusted to pH 10 with ammonium hydroxide)] to give the title compound (13 mg, 0.025 mmol, 90% yield). ¹H NMR (400 MHz, DMSO-d₆)) δ ppm 8.10 (dd, J=6.8, 2.2 Hz, 1H), 7.90 (t, J=6.9 Hz, 1H), 7.67-7.58 (m, 2H), 7.48 (td, J=8.9, 0.9 Hz, 1H), 7.22-7.13 (m, 1H), 7.05 (ddd, J=11.4, 2.9, 1.5 Hz, 1H), 6.83 (ddd, J=8.9, 2.8, 1.4 Hz, 1H), 5.09 (ddd, J=7.8, 5.4, 3.8 Hz, 1H), 4.49 (d, J=2.4 Hz, 2H), 3.54-3.45 (m, 2H), 3.04-2.88 (m, 2H), 2.13-1.91 (m, 2H), 1.64-1.52 (m, 2H), 1.40 (s, 1H), 1.38-1.21 (m, 3H); MS (ESI⁺) m/z 521 (M+H)⁺.

### Example 270: (2S,4R)-6-chloro-N-{(1RS,2SR,4RS,5SR)-5-[2-(4-chloro-3-fluorophenoxy)acetamido]-7-oxabicyclo[2.2.1]heptan-2-yl}-4-hydroxy-3,4-dihydro-2H-1-benzopyran-2-carboxamide (Compound 369)

### Example 270A: furan-3-ylmethanol

To a solution of furan-3-carboxylic acid (50 g, 446 mmol) in tetrahydrofuran (500 mL) was added a 1 N solution of borane in tetrahydrofuran (669 mL, 669 mmol) at 0° C., and the mixture was stirred at 20° C. for 1 hour. One additional vial on 25 g scale and six additional vials on 50 g scale were set up as described above. The reactions conducted in parallel were combined for work up. After cooling to 0° C., the reaction mixture was quenched with water until gas evolution had ceased. After bulk solvent removal, the resulting crude residue was then partitioned between saturated aqueous NaHCO₃and ethyl acetate, and the aqueous layer was further extracted with ethyl acetate (2×1000 mL). The combined organic phases were washed with brine (1000 mL), dried Na₂SO₄, and concentrated to dryness under reduced pressure. The residue was purified by column chromatography on silica gel with petroleum ether: ethyl acetate=3:1 to give the title compound (230 g, yield 63.1%). ¹HNMR (400 MHz, DMSO-d₆) δ ppm 7.46-7.61 (m, 2H), 4.34 (d, J=5.50 Hz, 2H), 4.97 (t, J=5.50 Hz, 1H), 6.44 (d, J=0.63 Hz, 1H).

### Example 270B: 3-((benzyloxy)methyl)furan

To a solution of Example 270A (20 g, 183 mmol) in N,N-dimethylformamide (200 mL) was added NaH (8.81 g, 220 mmol) at 0° C. and the mixture was stirred at 0° C. for 0.5 hour. (Bromomethyl)benzene (37.7 g, 220 mmol) was added to the reaction mixture at 0° C. and stirred at 20° C. for 12 hours. One additional vial on 5 g scale and nine additional vials on 20 g scale were set up as described above. The reactions conducted in parallel were combined for work up. After cooling to 0° C., the reaction was quenched with water until gas evolution had ceased. The mixture was extracted with ethyl acetate (3×3000 mL). The combined organic fractions were washed with brine (2×1000 mL), dried over Na₂SO₄, filtered and concentrated under reduced pressure. The residue was purified by column chromatography on silica gel eluted with petroleum ether: ethyl acetate=100:1 to 50:1 to give the title compound (480 g, yield 91%). ¹H NMR (400 MHz, CDCl₃) δ ppm 7.27 (s, 7H), 6.37 (s, 1H), 4.45 (s, 2H), 4.35 (s, 2H).

### Example 270C: rac-(1R,2R,4R)-5-((benzyloxy)methyl)-7-oxabicyclo[2.2.1]hept-5-ene-2-carbonitrile

Acrylonitrile (33.8 g, 638 mmol) was treated portionwise with zinc chloride (20.85 g, 153 mmol), and the mixture was stirred at 20° C. for 10 minutes. Then the product of Example 270B (30 g, 128 mmol) was added to the mixture and the mixture was stirred at 20° C. for 12 hours. Fifteen additional vials on 30 g scale were set up as described above. The reactions conducted in parallel were combined for work up. The combined reaction mixtures were diluted with ethyl acetate (1000 mL), silica gel (1000 g) was added, and the resulting mixture was concentrated under reduced pressure. The residue was purified by column chromatography on silica gel eluted with petroleum ether: ether acetate=3:1 to give the title compound (129 g, yield 20.96%). ¹H NMR (400 MHz, DMSO-d₆) δ ppm 7.20-7.41 (m, 5H), 6.01-6.33 (m, 1H), 5.17-5.23 (m, 1H), 5.01-5.08 (m, 1H), 4.40-4.52 (m, 2H), 4.08-4.23 (m, 2H), 3.97-4.07 (m, 1H), 2.72 (dd, J=8.57, 3.81 Hz, 1H), 1.98 (s, 2H), 1.85-1.94 (m, 1H), 1.71-1.82 (m, 1H), 1.17 (t, J=7.13 Hz, 2H).

### Example 270D: rac-(1R,2R,4R)-5-((benzyloxy)methyl)-7-oxabicyclo[2.2.1]heptane-2-carbonitrile

To a solution of the product of Example 270C (15 g, 49.7 mmol) in methanol (150 mL) was added Pd/C (5.29 g, 2.487 mmol) under argon, and the mixture was stirred at 20° C. under hydrogen (15 psi) for 2 hours. One additional vial on 1 g scale and two additional vials on 15 g scale were set up as described above. The suspension was filtered through a pad of diatomaceous earth and the pad was washed with methanol (5×200 mL). The combined filtrates were concentrated to dryness and the residue was purified by column chromatography on silica gel eluted with petroleum ether: ethyl acetate=3:1 to give the title compound (38 g, yield 64.5%). ¹H NMR (400 MHz, DMSO-d₆) δ ppm 7.25-7.45 (m, 5H), 4.74-4.88 (m, 1H), 4.56-4.71 (m, 1H), 4.37-4.52 (m, 1H), 3.45-3.64 (m, 1H), 2.89-3.23 (m, 1H), 2.09-2.36 (m, 2H), 1.85-2.04 (m, 1H), 1.62-1.84 (m, 1H), 1.05 (dd, J=12.51, 5.50 Hz, 1H).

### Example 270E: rac-(1R,2S,4R)-5-((benzyloxy)methyl)-7-oxabicyclo[2.2.1]heptane-2-carboxylic Acid

To the solution of the product of Example 270D (27 g, 89 mmol) in ethanol (270 mL) was added 3 N aqueous solution of KOH (39.8 g, 710 mmol) at 20° C., and the mixture was stirred at 100° C. for 16 hours. One additional vial on 1 g scale and one additional vial on 10 g scale were set up as described above. The reactions conducted in parallel were combined for work up. The mixture was concentrated under reduced pressure and the residue was extracted with ethyl acetate (3×500 mL). The aqueous phase was adjusted to pH=1 with 1 N HCl solution. The mixture was extracted with ethyl acetate (3×500 mL), and the combined organic phases were concentrated under reduced pressure to give the title compound (35 g, yield 85%). ¹H NMR (400 MHz, DMSO-d₆) δ ppm 12.03-12.41 (m, 1H), 7.23-7.49 (m, 5H), 4.55-4.67 (m, 1H), 4.33-4.54 (m, 3H), 3.52 (dd, J=9.66, 6.36 Hz, 1H), 2.19-2.38 (m, 1H), 1.70-1.90 (m, 2H), 1.02 (dd, J=12.04, 5.20 Hz, 1H).

### Example 270F: tert-butyl (rac-(1R,2S,4R)-5-((benzyloxy)methyl)-7-oxabicyclo[2.2.1]heptan-2-yl)carbamate

To a solution of the product of Example 270E (14 g, 42.7 mmol) in toluene (140 mL) and t-butanol (10 mL, 105 mmol) was added diphenylphosphoryl azide (17.63 g, 64.0 mmol) and triethylamine (11.90 mL, 85 mmol) at 20° C., and the mixture was stirred at 100° C. for 16 hours under N₂. One additional vial on 1 g scale and two additional vials on 10 g scale were set up as described above. The reactions conducted in parallel were combined for work up. Silica gel (200 g) was added to the solution, and the resulting mixture was concentrated under reduced pressure. The residue was purified by column chromatography on silica gel eluted with petroleum ether:ethyl acetate=3:1 to give the title compound (15 g, yield 33.7%). ¹H NMR (400 MHz, CDCl₃) δ ppm 7.29-7.39 (m, 5H), 4.71 (br d, J=6.50 Hz, 1H), 4.42-4.61 (m, 3H), 4.24-4.36 (m, 1H), 3.60-3.72 (m, 1H), 3.47-3.58 (m, 1H), 3.15-3.33 (m, 1H), 2.40 (tq, J=10.43, 5.14 Hz, 1H), 2.23 (br dd, J=13.45, 8.07 Hz, 1H), 1.89 (td, J=11.94, 6.00 Hz, 1H), 1.35-1.53 (m, 9H), 1.29-1.33 (m, 1H), 0.81-0.98 (m, 1H).

### Example 270G: tert-butyl (rac-(1R,2S,4R)-5-(hydroxymethyl)-7-oxabicyclo[2.2.1]heptan-2-yl)carbamate

To the solution of the product of Example 270F (3 g, 7.20 mmol) in tetrahydrofuran (30 mL) was added 10% Pd(OH)₂ on carbon (1.011 g, 0.720 mmol) under N₂, and the mixture was stirred at 50° C. for 24 hours under H₂ at 50 psi. One additional vial on 0.3 g scale and one additional vial on 3 g were set up as described above. The reactions conducted in parallel were combined for work up. The suspension was filtered through a pad of diatomaceous earth and the pad was washed with tetrahydrofuran (3×50 mL). The combined filtrates were concentrated to dryness under reduced pressure to give the title compound (4 g, yield 82%). ¹H NMR (400 MHz, CDCl₃) δ ppm 4.77 (br d, J=7.38 Hz, 1H), 4.58 (t, J=5.07 Hz, 1H), 4.32 (br d, J=5.88 Hz, 1H), 3.65-3.84 (m, 3H), 3.48 (t, J=10.01 Hz, 1H), 2.19-2.40 (m, 2H), 1.83-1.95 (m, 3H), 1.44 (s, 9H), 1.34 (dt, J=13.45, 4.35 Hz, 1H), 0.87-1.00 (m, 1H).

### Example 270H: tert-butyl (rac-(1R,2S,4R)-5-cyano-7-oxabicyclo[2.2.1]heptan-2-yl)carbamate

To a solution of the product of Example 270G (3.5 g, 8.63 mmol) in acetonitrile (180 mL) and water (20 mL) was added TEMPO (0.067 g, 0.432 mmol), iodobenzene diacetate (6.12 g, 18.99 mmol) and ammonium acetate (2.66 g, 34.5 mmol) at 20° C., and the mixture was stirred at 20° C. for 3 hours. One additional vial on 0.5 g scale was set up as described above. The reactions conducted in parallel were combined for work up. The solvent was removed under reduced pressure and the residue was partitioned between water (50 mL) and ethyl acetate (50 mL). The organic layer was separated, dried over Na₂SO₄ and filtered. The filtrate was concentrated under reduced pressure and the residue was purified by column chromatography on silica gel eluted with petroleum ether: ethyl acetate=4:1 to get the title compound (2.6 g, yield 94%). ¹H NMR (400 MHz, CDCl₃) δ ppm 4.75 (t, J=5.07 Hz, 1H), 4.65 (br s, 1H), 4.51 (br d, J=5.63 Hz, 1H), 3.94 (br s, 1H), 2.77-2.87 (m, 1H), 2.62 (dd, J=14.01, 8.13 Hz, 1H), 2.22 (td, J=12.35, 5.82 Hz, 1H), 1.74 (br dd, J=12.82, 5.32 Hz, 1H), 1.49-1.52 (m, 1H), 1.41-1.48 (m, 9H), 1.23-1.33 (m, 1H), 0.94-1.01 (m, 1H), 0.83-0.92 (m, 2H).

### Example 2701: rac-(1R,2S,4R,5S)-5-((tert-butoxycarbonyl)amino)-7-oxabicyclo[2.2.1]heptane-2-carboxylic Acid

To a solution of the product of Example 270H (1.3 g, 4.64 mmol) in ethanol (20 mL) was added a solution of KOH (12.37 mL, 37.1 mmol, 3 mol/L in water) at 20° C., and the mixture was stirred at 100° C. for 16 hours. One additional vial on 1.3 g scale was set up as described above. The reactions conducted in parallel were combined for work up. The mixture was cooled to 20° C., and extracted with ethyl acetate (3×100 mL). The aqueous phase was adjusted to pH=1 with HCl (1 mol/L in water) and then extracted with ethyl acetate (5×50 mL). The combined organic fractions were concentrated under reduced pressure to give the title compound (2.2 g, yield 83%). ¹H NMR (400 MHz, DMSO-d₆) δ ppm 12.14 (br s, 2H), 7.21-7.43 (m, 6H), 4.60 (d, J=5.63 Hz, 1H), 4.39-4.52 (m, 4H), 3.47-3.56 (m, 1H), 2.51-2.57 (m, 2H), 2.16-2.34 (m, 2H), 1.70-1.89 (m, 3H), 1.02 (dd, J=11.94, 5.19 Hz, 1H).

### Example 270J: benzyl tert-butyl rac-(R,2S,4R,5S)-7-oxabicyclo[2.2.1]heptane-2,5-diyldicarbamate

To a solution of the product of Example 2701 (2 g, 7.00 mmol) in toluene (20 mL) was added diphenylphosphoryl azide (2.89 g, 10.49 mmol) and triethylamine (1.950 mL, 13.99 mmol) at 20° C., and the mixture was stirred at 90° C. for 2 hours. Then phenylmethanol (1.084 mL, 10.49 mmol) was added and the mixture was stirred at 110° C. for 16 hours. One additional vial on 0.2 g scale was set up as described above. The reactions conducted in parallel were combined for work up. The mixture was concentrated and the residue was purified by flash chromatographyl on silica gel (0-70% ethyl acetate in petroleum ether on 40 g column) to give the title compound (1.9 g, yield 68.1%). ¹H NMR (400 MHz, CDCl₃) δ ppm 7.28-7.39 (m, 5H), 4.70 (br d, J=6.88 Hz, 1H), 4.40-4.61 (m, 3H), 4.25-4.34 (m, 1H), 3.67 (br s, 1H), 3.48-3.58 (m, 1H), 3.19-3.30 (m, 1H), 2.40 (tt, J=10.27, 5.30 Hz, 1H), 2.24 (br dd, J=13.51, 8.13 Hz, 1H), 1.82-1.96 (m, 1H), 1.39-1.50 (m, 10H), 1.24-1.35 (m, 1H), 0.92 (br dd, J=12.57, 5.57 Hz, 1H).

### Example 270K: tert-butyl (rac-(1R,2S,4R,5S)-5-amino-7-oxabicyclo[2.2.1]heptan-2-yl)carbamate

To a solution of the product of Example 270J (0.9 g, 2.235 mmol) in tetrahydrofuran (30 mL) was added 10% Pd(OH)₂ on carbon (0.314 g, 0.223 mmol) under N₂ at 20° C., and the mixture was stirred at 30° C. under H2 (30 psi) for 16 hours. One additional vial on 0.1 g scale and one additional vial on 0.9 g scale were set up as described above. The combined reaction mixtures were filtered through a pad of diatomaceous earth and the pad was washed with tetrahydrofuran (4×30 mL). The combined filtrate was concentrated to dryness, and the residue was purified by HPLC (performed on a Gilson 281 semi-preparative HPLC system using a Phenomenex® Luna® C18(2) 10 μm 100 Å AXIA™ column (250 mm×80 mm) column. A gradient of acetonitrile (A) and 0.075% trifluoroacetic acid in water (B) is used, at a flow rate of 80 mL/minute. A linear gradient is used from about 30% of A to about 100% of A over about 30 minutes. Detection method is UV at wave length of 220 nm and 254 nm) and lyophilized to give the title compound (1.05 g, yield 61.7%). ¹H NMR (400 MHz, DMSO-d₆) δ ppm 7.86 (br s, 2H), 6.85-6.96 (m, 1H), 4.44 (d, J=5.88 Hz, 1H), 4.36 (d, J=5.63 Hz, 1H), 3.40-3.44 (m, 1H), 3.40-3.44 (m, 1H), 3.34 (br s, 1H), 1.79-1.91 (m, 2H), 1.43-1.61 (m, 2H), 1.38 (s, 9H).

### Example 270L: tert-butyl (rac-(1R,2S,4R,5S)-5-(2-(4-chloro-3-fluorophenoxy)acetamido)-7-oxabicyclo[2.2.1]heptan-2-yl)carbamate

To a mixture of the product of Example 270K (0.326 g, 0.952 mmol), 2-(4-chloro-3-fluorophenoxy)acetic acid (0.214 g, 1.048 mmol), and N-ethyl-N-isopropylpropan-2-amine (0.582 mL, 3.33 mmol) in N,N-dimethylformamide (10.0 mL), 2-(3H-[1,2,3]triazolo[4,5-b]pyridin-3-yl)-1,1,3,3-tetramethylisouronium hexafluorophosphate (V) (0.453 g, 1.190 mmol) was added and the mixture was stirred at ambient temperature for 30 minutes. Water was added, and the suspension was stirred for 5 minutes. The precipitate was collected by filtration, washed with water and air dried to give 220 mg of the title compound. ¹H NMR (400 MHz, DMSO-d₆) δ ppm 8.06 (d, J=6.9 Hz, 1H), 7.48 (t, J=8.9 Hz, 1H), 7.05 (dq, J=11.4, 3.1 Hz, 1H), 6.88-6.79 (m, 2H), 4.51 (s, 2H), 4.24 (dd, J=17.6, 5.8 Hz, 2H), 3.78 (td, J=7.6, 3.2 Hz, 1H), 3.46 (s, 1H), 1.81 (ddd, J=17.0, 12.9, 8.1 Hz, 2H), 1.52-1.42 (m, 2H), 1.38 (s, 9H).

### Example 270M: N-(rac-(1R,2S,4R,5S)-5-amino-7-oxabicyclo[2.2.1]heptan-2-yl)-2-(4-chloro-3-fluorophenoxy)acetamide trifluoroacetic Acid

To a solution of the product of Example 270L (0.22 g, 0.530 mmol) in dichloromethane (5.0 mL) was added 2,2,2-trifluoroacetic acid (2.5 mL, 32.4 mmol). The reaction mixture was stirred at ambient temperature for one hour. Solvent and excess 2,2,2-trifluoroacetic acid were removed under high vacuum to give 0.24 g of the title compound. ¹H NMR (400 MHz, DMSO-d₆) δ ppm 8.16 (d, J=6.7 Hz, 1H), 7.95-7.89 (m, 3H), 7.49 (t, J=8.9 Hz, 1H), 7.06 (dd, J=11.4, 2.9 Hz, 1H), 6.84 (ddd, J=9.0, 2.9, 1.2 Hz, 1H), 4.64-4.47 (m, 1H), 4.53 (s, 2H), 4.50-4.39 (m, 1H), 3.84 (ddd, J=8.0, 6.7, 3.0 Hz, 1H), 3.39 (ddt, J=9.7, 6.8, 3.5 Hz, 1H), 1.95 (ddd, J=13.6, 8.0, 5.5 Hz, 2H), 1.62 (ddd, J=13.2, 6.2, 3.0 Hz, 1H), 1.52 (ddd, J=13.6, 6.4, 2.3 Hz, 1H).

### Example 270N: (2S,4R)-6-chloro-4-hydroxychroman-2-carboxylic Acid

The product of Example 261A (140 mg, 0.612 mmol) was combined with trifluoroacetic acid (1.0 mL) and stirred at 30° C. for 2 hours. The reaction mixture was concentrated under high vacuum. The residue was taken up in acetonitrile (3.0 mL), and aqueous ammonium hydroxide (3 M, 3 mL) was added. The resulting mixture was stirred at ambient temperature for 18 hours and then concentrated under high vacuum. The residue was taken up in methanol, filtered through a glass microfiber frit and purified by preparative HPLC [Waters SunFire™ C18 5 μm OBD column, 30×150 mm, flow rate 30 mL/minute, 3-100% gradient of acetonitrile in buffer (0.1% trifluoroacetic acid)] to give the title compound (80 mg, 0.35 mmol, 57% yield). MS (ESI⁻) m/z 227 (M−H)⁻.

### Example 2700: (2S,4R)-6-chloro-N-{(1RS,2SR,4RS,5SR)-5-[2-(4-chloro-3-fluorophenoxy)acetamido]-7-oxabicyclo[2.2.1]heptan-2-yl}-4-hydroxy-3,4-dihydro-2H-1-benzopyran-2-carboxamide

To a solution of the product of Example 270M (21.43 mg, 0.050 mmol), the product of Example 270N (12 mg, 0.052 mmol) and N-ethyl-N-isopropylpropan-2-amine (0.035 mL, 0.200 mmol) in N,N-dimethylformamide (1 mL) was added 2-(3H-[1,2,3]triazolo[4,5-b]pyridin-3-yl)-1,1,3,3-tetramethylisouronium hexafluorophosphate (V) (28.5 mg, 0.075 mmol) and the mixture was stirred at ambient temperature for 30 minutes. Solvent was removed under high vacuum and the residue was purified by HPLC (Phenomenex® Luna® C18(2) 10 μm 100 Å AXIA™ column (250 mm×50 mm). A 30-100% gradient of acetonitrile (A) and 0.1% trifluoroacetic acid in water (B) was used over 25 minutes, at a flow rate of 50 mL/minute) to give 17 mg of the title compound. ¹H NMR (500 MHz, DMSO-d₆) δ ppm 8.11 (dd, J=6.8, 2.9 Hz, 1H), 8.04 (dd, J=10.6, 6.7 Hz, 1H), 7.49 (td, J=8.9, 1.3 Hz, 1H), 7.31 (d, J=2.6 Hz, 1H), 7.24 (dt, J=8.8, 2.3 Hz, 1H), 7.06 (ddd, J=11.4, 2.9, 1.5 Hz, 1H), 6.93 (d, J=8.7 Hz, 1H), 6.87-6.81 (m, 1H), 4.65-4.55 (m, 2H), 4.53 (s, 2H), 4.39-4.29 (m, 2H), 3.90-3.78 (m, 2H), 2.07 (dq, J=13.9, 3.6 Hz, 1H), 1.92 (ddt, J=16.6, 12.5, 6.0 Hz, 3H), 1.57 (dddt, J=29.4, 12.8, 6.3, 3.3 Hz, 2H); MS (APCI⁺) m/z 525.50 (M+H)⁺.

### Example 271: (2R,4R)-6-chloro-N-{(1RS,2SR,4RS,5SR)-5-[2-(4-chloro-3-fluorophenoxy)acetamido]-7-oxabicyclo[2.2.1]heptan-2-yl}-4-hydroxy-3,4-dihydro-2H-1-benzopyran-2-carboxamide (Compound 370)

### Example 271A: (2R)-6-chloro-N-{(1RS,2SR,4RS,5SR)-5-[2-(4-chloro-3-fluorophenoxy)acetamido]-7-oxabicyclo[2.2.1]heptan-2-yl}-4-oxo-3,4-dihydro-2H-1-benzopyran-2-carboxamide

To a solution of the product of Example 270M (40 mg, 0.093 mmol), the product of Example 124A (23.26 mg, 0.103 mmol) and N-ethyl-N-isopropylpropan-2-amine (0.065 mL, 0.373 mmol) in N,N-dimethylformamide (1 mL) was added 2-(3H-[1,2,3]triazolo[4,5-b]pyridin-3-yl)-1,1,3,3-tetramethylisouronium hexafluorophosphate (V) (44.3 mg, 0.117 mmol) and the mixture was stirred at ambient temperature for 30 minutes. Solvent was removed under high vacuum and the residue was purified by HPLC (Phenomenex® Luna® C18(2) 10 μm 100 Å AXIA™ column (250 mm×50 mm). A 30-100% gradient of acetonitrile (A) and 0.1% trifluoroacetic acid in water (B) was used over 25 minutes, at a flow rate of 50 mL/minute) to give 38 mg of the title compound. ¹H NMR (600 MHz, DMSO-d₆) δ ppm 8.31 (d, J=6.8 Hz, 1H), 8.09 (dd, J=6.8, 5.4 Hz, 1H), 7.66-7.59 (m, 2H), 7.48 (td, J=8.9, 0.7 Hz, 1H), 7.16 (ddd, J=8.7, 2.3, 0.5 Hz, 1H), 7.06 (ddd, J=11.4, 2.9, 1.1 Hz, 1H), 6.83 (ddt, J=8.9, 2.6, 1.2 Hz, 1H), 5.12 (ddd, J=8.6, 5.5, 3.8 Hz, 1H), 4.57-4.48 (m, 2H), 4.33-4.23 (m, 2H), 3.84-3.74 (m, 2H), 3.02-2.90 (m, 2H), 1.90 (tdd, J=12.9, 7.0, 3.7 Hz, 2H), 1.56-1.47 (m, 2H).

### Example 271B: (2R,4R)-6-chloro-N-{(1RS,2SR,4RS,5SR)-5-[2-(4-chloro-3-fluorophenoxy)acetamido]-7-oxabicyclo[2.2.1]heptan-2-yl}-4-hydroxy-3,4-dihydro-2H-1-benzopyran-2-carboxamide

A mixture of the product of Example 271A (0.037 g, 0.071 mmol) and sodium tetrahydroborate (0.021 g, 0.566 mmol) in methanol (2.5 mL) was stirred at ambient temperature for 15 minutes. Solvent was removed under high vacuum and the residue was purified by HPLC (Phenomenex® Luna® C18(2) 10 μm 100 Å AXIA™ column (250 mm×50 mm). A 20-90% gradient of acetonitrile (A) and 0.1% trifluoroacetic acid in water (B) was used over 25 minutes, at a flow rate of 50 mL/minute) to give 23 mg of the title compound. ¹H NMR (500 MHz, DMSO-d₆) δ ppm 8.11 (d, J=6.8 Hz, 1H), 7.96 (dd, J=14.8, 6.8 Hz, 1H), 7.49 (td, J=8.9, 1.1 Hz, 1H), 7.38 (dd, J=2.6, 1.2 Hz, 1H), 7.19 (dt, J=8.8, 2.9 Hz, 1H), 7.06 (ddd, J=11.4, 2.9, 1.2 Hz, 1H), 6.91-6.81 (m, 2H), 5.69 (s, 1H), 4.80 (ddd, J=9.7, 6.1, 2.3 Hz, 1H), 4.65 (dt, J=11.8, 2.5 Hz, 1H), 4.53 (s, 2H), 4.39-4.33 (m, 1H), 4.31 (d, J=5.8 Hz, 1H), 3.83 (tt, J=7.0, 3.2 Hz, 2H), 2.36-2.27 (m, 1H), 1.96-1.86 (m, 2H), 1.81-1.70 (m, 1H), 1.61 (s, 1H), 1.53 (s, 1H); MS (APCI⁺) m/z 525.06 (M+H)⁺.

### Example 272: (2R,4R)-6-chloro-N-{(1RS,2SR,4RS,5SR)-5-[2-(4-chloro-3-fluorophenoxy)acetamido]bicyclo[2.2.1]heptan-2-yl}-4-hydroxy-3,4-dihydro-2H-1-benzopyran-2-carboxamide (Compound 371)

The reaction and purification conditions described in Example 108F substituting the product of Example 269 for the product of Example 108E gave the title compound. ¹H NMR (500 MHz, DMSO-d₆) δ ppm 7.94 (d, J=7.0 Hz, 1H), 7.84 (dd, J=7.0, 3.9 Hz, 1H), 7.49 (td, J=8.8, 1.0 Hz, 1H), 7.40-7.36 (m, 1H), 7.23-7.16 (m, 1H), 7.06 (ddd, J=11.4, 2.9, 1.1 Hz, 1H), 6.88 (d, J=8.7 Hz, 1H), 6.87-6.81 (m, 1H), 5.71 (s, 1H), 4.80 (dd, J=10.7, 5.9 Hz, 1H), 4.64-4.57 (m, 1H), 4.55-4.45 (m, 2H), 3.58-3.51 (m, 2H), 2.34-2.26 (m, 1H), 2.16-2.06 (m, 2H), 1.80-1.69 (m, 1H), 1.66-1.57 (m, 2H), 1.47-1.32 (m, 4H); MS (APCI⁺) m/z 505 (M−H₂O+H)⁺.

### Example 273: (2S,4S)-6-chloro-N-{(1RS,2SR,4RS,5SR)-5-[2-(4-chloro-3-fluorophenoxy)acetamido]-7-oxabicyclo[2.2.1]heptan-2-yl}-4-hydroxy-3,4-dihydro-2H-1-benzopyran-2-carboxamide (Compound 372)

The title compound was synthesized using the same procedure as described in Example 271A through Example 271B substituting the product of Example 124A with the product of Example 125A. ¹H NMR (500 MHz, DMSO-d₆) δ ppm 8.11 (d, J=6.8 Hz, 1H), 7.96 (dd, J=14.8, 6.8 Hz, 1H), 7.49 (td, J=8.9, 1.1 Hz, 1H), 7.38 (dd, J=2.6, 1.2 Hz, 1H), 7.19 (dt, J=8.7, 2.9 Hz, 1H), 7.06 (ddd, J=11.4, 2.9, 1.2 Hz, 1H), 6.88 (dd, J=8.7, 1.5 Hz, 1H), 6.86-6.81 (m, 1H), 5.69 (s, 1H), 4.80 (ddd, J=10.4, 6.0, 2.4 Hz, 1H), 4.65 (dt, J=11.9, 2.5 Hz, 1H), 4.53 (s, 2H), 4.39-4.33 (m, 1H), 4.31 (d, J=5.8 Hz, 1H), 3.83 (tt, J=5.8, 2.8 Hz, 2H), 2.36-2.27 (m, 1H), 1.96-1.86 (m, 2H), 1.81-1.70 (m, 1H), 1.64-1.50 (m, 2H); MS (APCI⁺) m/z 525.02 (M+H)⁺.

### Example 274: N-(4-{[(2R,4R)-6-chloro-4-hydroxy-3,4-dihydro-2H-1-benzopyran-2-carbonyl]amino}bicyclo[2.1.1]hexan-1-yl)-5-(trifluoromethoxy)pyridine-2-carboxamide (Compound 373)

### Example 274A: tert-butyl (4-{[(2R)-6-chloro-4-oxo-3,4-dihydro-2H-1-benzopyran-2-carbonyl]amino}bicyclo[2.1.1]hexan-1-yl)carbamate

tert-Butyl (4-aminobicyclo[2.1.1]hexan-1-yl)carbamate (0.15 g, 0.71 mmol, Matrix) and the product of Example 124A (0.16 g, 0.71 mmol) were combined with N,N-dimethylformamide (5 mL). Triethylamine (0.295 mL, 2.12 mmol) and 1-[bis(dimethylamino)methylene]-1H-1,2,3-triazolo[4,5-b]pyridinium 3-oxid hexafluorophosphate (349 mg, 0.92 mmol) were added sequentially. The reaction mixture was stirred at ambient temperature for 1 hour and then water (1 mL) was added. The resulting solution was filtered through a glass microfiber frit and purified by preparative HPLC [YMC TriArt™ C18 Hybrid 5 μm column, 50×100 mm, flow rate 140 mL/minute, 5-100% gradient of acetonitrile in buffer (0.025 M aqueous ammonium bicarbonate, adjusted to pH 10 with ammonium hydroxide)] to give the title compound (0.25 g, 0.59 mmol, 84% yield). MS (APCI⁺) m/z 365 (M−C(CH₃)₃+H)⁺.

### Example 274B: N-(4-{[(2R)-6-chloro-4-oxo-3,4-dihydro-2H-1-benzopyran-2-carbonyl]amino}bicyclo[2.1.1]hexan-1-yl)-5-(trifluoromethoxy)pyridine-2-carboxamide

Trifluoroacetic acid (0.5 mL, 6.49 mmol) was added to the product of Example 274A (36 mg, 0.086 mmol). After stirring at ambient temperature for 15 minutes, the mixture was concentrated under reduced pressure. To the residue was added N,N-dimethylformamide (0.8 mL), triethylamine (0.083 ml, 0.60 mmol), 5-(trifluoromethoxy)picolinic acid (18 mg, 0.086 mmol) and 1-[bis(dimethylamino)methylene]-1H-1,2,3-triazolo[4,5-b]pyridinium 3-oxid hexafluorophosphate (39 mg, 0.10 mmol) in sequential order. The resulting mixture was stirred at ambient temperature for 30 minutes. Water (0.1 mL) was added, and the resulting solution was filtered through a glass microfiber frit and purified by preparative HPLC [YMC TriArt™ C18 Hybrid 5 μm column, 50×100 mm, flow rate 140 mL/minute, 5-100% gradient of acetonitrile in buffer (0.025 M aqueous ammonium bicarbonate, adjusted to pH 10 with ammonium hydroxide)] to give the title compound (35 mg, 0.069 mmol, 80% yield). MS (APCI⁺) m/z 510 (M+H)⁺.

### Example 274C: N-(4-{[(2R,4R)-6-chloro-4-hydroxy-3,4-dihydro-2H-1-benzopyran-2-carbonyl]amino}bicyclo[2.1.1]hexan-1-yl)-5-(trifluoromethoxy)pyridine-2-carboxamide

The product of Example 274B (35 mg, 0.069 mmol) was combined with methanol (1.0 mL) and stirred at ambient temperature. Sodium borohydride (23.4 mg, 0.62 mmol) was added. After stirring at ambient temperature for 30 minutes, saturated ammonium chloride solution (0.1 mL) was added. The mixture was stirred for 10 minutes, combined with diatomaceous earth (about 5 grams), and concentrated under reduced pressure to a free flowing powder. The powder was directly purified by reversed-phase flash chromatography [Custom packed YMC TriArt™ C18 Hybrid 20 μm column, 25×150 mm, flow rate 70 mL/minute, 5-100% gradient of acetonitrile in buffer (0.025 M aqueous ammonium bicarbonate, adjusted to pH 10 with ammonium hydroxide)] to give the title compound (30 mg, 0.059 mmol, 85% yield). ¹H NMR (500 MHz, DMSO-d₆) δ ppm 9.09 (s, 1H), 8.73-8.68 (m, 1H), 8.47 (s, 1H), 8.18-8.12 (m, 1H), 8.11-8.05 (m, 1H), 7.39 (dd, J=2.6, 1.0 Hz, 1H), 7.20 (ddd, J=8.6, 2.7, 0.7 Hz, 1H), 6.90 (d, J=8.7 Hz, 1H), 5.71 (s, 1H), 4.81 (dd, J=10.8, 5.9 Hz, 1H), 4.61 (dd, J=11.9, 2.2 Hz, 1H), 2.35 (ddd, J=12.9, 5.9, 2.3 Hz, 1H), 2.17-2.13 (m, 2H), 1.99-1.94 (m, 2H), 1.94-1.84 (m, 4H), 1.73 (ddd, J=12.9, 12.0, 10.8 Hz, 1H); MS (APCI⁺) m/z 494 (M−H₂O+H)⁺.

### Example 275: (2R,4R)-6-chloro-4-hydroxy-N-{4-[4-(trifluoromethyl)benzamido]bicyclo[2.1.1]hexan-1-yl}-3,4-dihydro-2H-1-benzopyran-2-carboxamide (Compound 374)

The reaction and purification conditions described in Examples 274B through 274C substituting 4-(trifluoromethyl)benzoic acid for 5-(trifluoromethoxy)picolinic acid gave the title compound. ¹H NMR (500 MHz, DMSO-d₆) δ ppm 9.03 (s, 1H), 8.46 (s, 1H), 8.04 (d, J=8.1 Hz, 2H), 7.84 (d, J=8.2 Hz, 2H), 7.39 (dd, J=2.7, 1.0 Hz, 1H), 7.23-7.17 (m, 1H), 6.90 (d, J=8.7 Hz, 1H), 5.71 (s, 1H), 4.85-4.78 (m, 1H), 4.61 (dd, J=11.9, 2.2 Hz, 1H), 2.35 (ddd, J=12.9, 5.8, 2.2 Hz, 1H), 2.18-2.13 (m, 2H), 1.97-1.84 (m, 6H), 1.79-1.68 (m, 1H); MS (APCI⁺) m/z 495 (M+H)⁺.

### Example 276: (2R,4R)-6-chloro-N-{(1S,2R,4S,5R)-5-[2-(4-chloro-3-fluorophenoxy)acetamido]-7-oxabicyclo[2.2.1]heptan-2-yl}-4-hydroxy-3,4-dihydro-2H-1-benzopyran-2-carboxamide (Compound 375)

### Example 276A: tert-butyl ((1S,2R,4S,5R)-5-(2-(4-chloro-3-fluorophenoxy)acetamido)-7-oxabicyclo[2.2.1]heptan-2-yl)carbamate

The title compound was isolated from the product of Example 270L by preparative chiral HPLC [Chiralpak© AD-H 5 μm column, 20×250 mm, flow rate 6 mL/minute, 80% ethanol in heptane (isocratic gradient)] as the earlier eluting fraction. MS (APCI⁺) m/z 414.71 (M+H)⁺.

### Example 276B: N-((1S,2R,4S,5R)-5-amino-7-oxabicyclo[2.2.1]heptan-2-yl)-2-(4-chloro-3-fluorophenoxy)acetamide trifluoroacetic Acid

The title compound was synthesized using the same procedure as described in Example 270M substituting the product of Example 270L with the product of Example 276A. MS (APCI⁺) m/z 315.04 (M+H)⁺.

### Example 276C: (2R,4R)-6-chloro-N-{(1S,2R,4S,5R)-5-[2-(4-chloro-3-fluorophenoxy)acetamido]-7-oxabicyclo[2.2.1]heptan-2-yl}-4-hydroxy-3,4-dihydro-2H-1-benzopyran-2-carboxamide

The title compound was synthesized using the same procedure as described in Example 271A through Example 271B substituting the product of Example 271A with the product of Example 276B. ¹H NMR (500 MHz, DMSO-d₆) δ ppm 8.11 (d, J=6.9 Hz, 1H), 7.95 (d, J=6.8 Hz, 1H), 7.49 (t, J=8.9 Hz, 1H), 7.38 (dd, J=2.7, 1.0 Hz, 1H), 7.19 (ddd, J=8.8, 2.7, 0.7 Hz, 1H), 7.06 (dd, J=11.4, 2.8 Hz, 1H), 6.88 (d, J=8.7 Hz, 1H), 6.84 (ddd, J=9.0, 2.9, 1.2 Hz, 1H), 5.70 (d, J=6.4 Hz, 1H), 4.80 (dt, J=11.6, 6.1 Hz, 1H), 4.65 (dd, J=11.8, 2.3 Hz, 1H), 4.53 (d, J=1.0 Hz, 2H), 4.36 (d, J=5.8 Hz, 1H), 4.31 (d, J=5.8 Hz, 1H), 3.86-3.79 (m, 2H), 3.31 (s, 2H), 2.31 (ddd, J=12.9, 5.9, 2.3 Hz, 1H), 1.91 (dt, J=12.9, 7.8 Hz, 2H), 1.76 (ddd, J=12.9, 11.7, 10.6 Hz, 1H), 1.57 (dddd, J=27.7, 12.9, 5.9, 3.0 Hz, 2H); MS (APCI⁺) m/z 525.07 (M+H)⁺.

### Example 277: N-(3-{[(2R,4R)-6-chloro-4-hydroxy-3,4-dihydro-2H-1-benzopyran-2-carbonyl]amino}bicyclo[1.1.1]pentan-1-yl)-5-(trifluoromethoxy)pyridine-2-carboxamide (Compound 376)

The reaction and purification conditions described in Example 274C substituting the product of Example 280 for the product of Example 274B gave the title compound. ¹H NMR (400 MHz, DMSO-d₆) δ ppm 9.35 (s, 1H), 8.73-8.67 (m, 2H), 8.18-8.11 (m, 1H), 8.11-8.04 (m, 1H), 7.38 (dd, J=2.7, 1.0 Hz, 1H), 7.20 (ddd, J=8.7, 2.7, 0.7 Hz, 1H), 6.89 (d, J=8.7 Hz, 1H), 5.70 (d, J=5.5 Hz, 1H), 4.85-4.78 (m, 1H), 4.61 (dd, J=12.0, 2.2 Hz, 1H), 2.37 (s, 6H), 2.36-2.31 (m, 1H), 1.78-1.64 (m, 1H); MS (APCI⁺) m/z 480 (M−H₂O+H)⁺.

### Example 278: N-(3-{[(2R,4R)-6-chloro-4-hydroxy-3,4-dihydro-2H-1-benzopyran-2-carbonyl]amino}bicyclo[1.1.1]pentan-1-yl)-6-(trifluoromethoxy)pyridine-3-carboxamide (Compound 377)

The reaction and purification conditions described in Example 274C substituting the product of Example 281 for the product of Example 274B gave the title compound. ¹H NMR (500 MHz, DMSO-d₆) δ ppm 9.30 (s, 1H), 8.82-8.72 (m, 2H), 8.38 (dd, J=8.5, 2.5 Hz, 1H), 7.42-7.36 (m, 2H), 7.21 (ddd, J=8.7, 2.7, 0.7 Hz, 1H), 6.89 (d, J=8.7 Hz, 1H), 5.71 (d, J=5.6 Hz, 1H), 4.85-4.77 (m, 1H), 4.61 (dd, J=12.0, 2.2 Hz, 1H), 2.37 (s, 6H), 2.39-2.32 (m, 1H), 1.76-1.65 (m, 1H); MS (APCI⁺) m/z 480 (M−H₂O+H)⁺.

### Example 279: (2R,4R)-6-chloro-N-[3-(4-chloro-3-fluorobenzamido)bicyclo[1.1.1]pentan-1-yl]-4-hydroxy-3,4-dihydro-2H-1-benzopyran-2-carboxamide (Compound 378)

The reaction and purification conditions described in Examples 274A through 274C substituting tert-butyl (3-aminobicyclo[1.1.1]pentan-1-yl)carbamate for tert-butyl (4-aminobicyclo[2.1.1]hexan-1-yl)carbamate, and 4-chloro-3-fluorobenzoic acid for 5-(trifluoromethoxy)picolinic acid gave the title compound. ¹H NMR (500 MHz, DMSO-d₆) δ ppm 9.20 (s, 1H), 8.73 (s, 1H), 7.87-7.81 (m, 1H), 7.75-7.68 (m, 2H), 7.39 (dd, J=2.7, 1.0 Hz, 1H), 7.21 (ddd, J=8.7, 2.7, 0.7 Hz, 1H), 6.89 (d, J=8.7 Hz, 1H), 5.72 (s, 1H), 4.81 (dd, J=10.7, 5.9 Hz, 1H), 4.61 (dd, J=12.0, 2.3 Hz, 1H), 2.40-2.32 (m, 1H), 2.36 (s, 6H), 1.71 (ddd, J=12.9, 12.0, 10.8 Hz, 1H); MS (APCI⁺) m/z 447 (M−H₂O+H)⁺.

### Example 280: N-(3-{[(2R)-6-chloro-4-oxo-3,4-dihydro-2H-1-benzopyran-2-carbonyl]amino}bicyclo[1.1.1]pentan-1-yl)-5-(trifluoromethoxy)pyridine-2-carboxamide (Compound 379)

The reaction and purification conditions described in Examples 274A through 274B substituting tert-butyl (3-aminobicyclo[1.1.1]pentan-1-yl)carbamate for tert-butyl (4-aminobicyclo[2.1.1]hexan-1-yl)carbamate gave the title compound. ¹H NMR (500 MHz, DMSO-d₆) δ ppm 9.37 (s, 1H), 8.98 (s, 1H), 8.70 (dt, J=2.7, 0.8 Hz, 1H), 8.16-8.12 (m, 1H), 8.10-8.05 (m, 1H), 7.67-7.63 (m, 2H), 7.18 (dd, J=8.4, 0.8 Hz, 1H), 5.11 (dd, J=7.6, 6.6 Hz, 1H), 3.01-2.90 (m, 2H), 2.34 (s, 6H); MS (APCI⁺) m/z 496 (M+H)⁺.

### Example 281: N-(3-{[(2R)-6-chloro-4-oxo-3,4-dihydro-2H-1-benzopyran-2-carbonyl]amino}bicyclo[1.1.1]pentan-1-yl)-6-(trifluoromethoxy)pyridine-3-carboxamide (Compound 380)

The reaction and purification conditions described in Examples 274A through 274B substituting tert-butyl (3-aminobicyclo[1.1.1]pentan-1-yl)carbamate for tert-butyl (4-aminobicyclo[2.1.1]hexan-1-yl)carbamate, and 6-(trifluoromethoxy)nicotinic acid for 5-(trifluoromethoxy)picolinic acid gave the title compound. ¹H NMR (400 MHz, DMSO-d₆) δ ppm 9.29 (s, 1H), 8.99 (s, 1H), 8.75 (dd, J=2.5, 0.7 Hz, 1H), 8.37 (dd, J=8.6, 2.5 Hz, 1H), 7.69-7.61 (m, 2H), 7.38 (dd, J=8.6, 0.7 Hz, 1H), 7.22-7.14 (m, 1H), 5.11 (t, J=7.1 Hz, 1H), 2.97 (d, J=7.1 Hz, 2H), 2.35 (s, 6H); MS (APCI⁺) m/z 496 (M+H)⁺.

### Example 282: N-(3-{[(2R,4R)-6-chloro-4-hydroxy-3,4-dihydro-2H-1-benzopyran-2-carbonyl]amino}bicyclo[1.1.1]pentan-1-yl)-1-[cis-3-(trifluoromethoxy)cyclobutyl]-1H-pyrazole-4-carboxamide (Compound 381)

### Example 282A: ethyl 1-(5,8-dioxaspiro[3.4]octan-2-yl)-1H-pyrazole-4-carboxylate

Ethyl 1H-pyrazole-4-carboxylate (536 mg, 3.82 mmol) and 2-bromo-5,8-dioxaspiro[3.4]octane (738 mg, 3.82 mmol) were dissolved in anhydrous N,N-dimethylformamide (8 mL) under a nitrogen atmosphere. Potassium iodide (317 mg, 1.912 mmol) and cesium carbonate (3114 mg, 9.56 mmol) were added. The reaction mixture was heated at 100° C. overnight. The mixture was partitioned between ethyl acetate (25 mL) and water (10 mL). The layers were separated, and the organic layer was washed with brine (4×5 mL). The organic layer was dried over MgSO₄, filtered and concentrated in vacuo. The residue was purified by chromatography on silica gel (0-50% ethyl acetate/isohexane) to afford the title compound (416 mg, 42% yield). ¹H NMR (500 MHz, DMSO-d₆) δ ppm 8.41 (s, 1H), 7.91 (s, 1H), 4.80 (p, J=8.0 Hz, 1H), 4.21 (q, J=7.1 Hz, 2H), 3.93-3.87 (m, 2H), 3.87-3.81 (m, 2H), 2.86-2.78 (m, 2H), 2.78-2.69 (m, 2H), 1.26 (t, J=7.1 Hz, 3H); MS (ESI) m/z 253 (M+H)⁺.

### Example 282B: ethyl 1-(3-oxocyclobutyl)-1H-pyrazole-4-carboxylate

To the product of Example 282A (416 mg, 1.649 mmol) in a mixture of dioxane (5 mL) and water (5 mL) was added pyridinium p-toluenesulfonate (2072 mg, 8.25 mmol) and the reaction mixture was stirred at 85° C. overnight. The reaction mixture was diluted with ethyl acetate (20 mL) and washed with water (3×10 mL). The organic layer was dried over MgSO₄, filtered and concentrated in vacuo to afford the title compound (287 mg, 82% yield). ¹H NMR (500 MHz, DMSO-d₆) δ ppm 8.59 (d, J=0.7 Hz, 1H), 7.95 (s, 1H), 5.25 (tt, J=8.0, 5.7 Hz, 1H), 4.23 (q, J=7.2 Hz, 2H), 3.68-3.50 (m, 4H), 1.27 (t, J=7.1 Hz, 3H); MS (ESI) m/z 209 (M+H)⁺.

### Example 282C: ethyl 1-(cis-3-hydroxycyclobutyl)-1H-pyrazole-4-carboxylate

To a stirred solution of the product of Example 282B (287 mg, 1.378 mmol) in tetrahydrofuran (6 mL), at −78° C. under an atmosphere of nitrogen was added lithium tri-tert-butoxyaluminum hydride in tetrahydrofuran (1 M, 1.7 mL, 1.700 mmol) slowly over 5 minutes and the resultant reaction mixture was stirred for 1 hour at this temperature. Additional lithium tri-tert-butoxyaluminum hydride in tetrahydrofuran (1 M, 0.2 mL, 0.200 mmol) was added and the reaction mixture was stirred for 30 minutes at −78° C. The reaction mixture was quenched with 1 M aqueous HCl (10 mL) and extracted with dichloromethane (3×10 mL). The combined organic fractions were dried over MgSO₄, filtered, and concentrated in vacuo. The residue was purified by chromatography on silica gel (0-100% ethyl acetate/isohexane) to afford the title compound (207 mg, 70% yield). ¹H NMR (500 MHz, DMSO-d₆) δ ppm 8.37 (s, 1H), 7.88 (s, 1H), 5.27 (d, J=6.9 Hz, 1H), 4.47-4.37 (m, 1H), 4.21 (q, J=7.1 Hz, 2H), 3.99-3.89 (m, 1H), 2.77-2.67 (m, 2H), 2.39-2.28 (m, 2H), 1.26 (t, J=7.1 Hz, 3H); MS (ESI) m/z 211 (M+H)⁺.

### Example 282D: ethyl 1-(cis-3-(trifluoromethoxy)cyclobutyl)-1H-pyrazole-4-carboxylate

A mixture of silver (I) trifluoromethanesulfonate (683 mg, 2.66 mmol), potassium fluoride (229 mg, 3.94 mmol), and Selectfluor™ (523 mg, 1.477 mmol) was stirred under a nitrogen atmosphere, in a flask wrapped with aluminum foil, and cooled with a water bath. To this was slowly added a solution of the product of Example 282C (207 mg, 0.985 mmol) in ethyl acetate (15 mL) followed by slow addition of 2-fluoropyridine (0.254 mL, 2.95 mmol) and then trimethyl(trifluoromethyl)silane (0.437 mL, 2.95 mmol). The reaction mixture was stirred at ambient temperature for 48 hours, and then filtered through a pad of diatomaceous earth. The filter cake was washed with ethyl acetate (20 mL) and the filtrate was concentrated in vacuo. The resulting residue was purified by chromatography on silica gel (0-100% ethyl acetate/isohexane) to afford the title compound (55 mg, 20% yield). ¹H NMR (500 MHz, DMSO-d₆) δ ppm 8.48 (s, 1H), 7.94 (s, 1H), 4.78 (p, J=7.2 Hz, 1H), 4.70-4.60 (m, 1H), 4.22 (q, J=7.1 Hz, 2H), 2.99-2.89 (m, 2H), 2.80-2.70 (m, 2H), 1.26 (t, J=7.1 Hz, 3H).

### Example 282E: 1-(cis-3-(trifluoromethoxy)cyclobutyl)-1H-pyrazole-4-carboxylic Acid

The product of Example 282D (53 mg, 0.190 mmol) was dissolved in tetrahydrofuran (0.5 mL) and methanol (0.25 mL) and lithium hydroxide hydrate (24 mg, 0.571 mmol) and water (0.25 mL) were added. The reaction mixture was stirred at ambient temperature overnight. 1 M aqueous HCl (4 mL) and dichloromethane (5 mL) were added and the layers were separated using a phase separator cartridge. The aqueous layer was re-extracted with dichloromethane (3 mL×2). The combined organic layers were concentrated under reduced pressure to afford the title compound (46 mg, 84% yield). ¹H NMR (500 MHz, DMSO-d₆) δ ppm 8.37 (s, 1H), 7.88 (s, 1H), 4.82-4.73 (m, 1H), 4.69-4.59 (m, 1H), 2.98-2.89 (m, 2H), 2.79-2.70 (m, 2H), one exchangeable proton not observed; MS (ESI) m/z 251 (M+H)⁺.

### Example 282F: tert-butyl (3-(1-(cis-3-(trifluoromethoxy)cyclobutyl)-1H-pyrazole-4-carboxamido)bicyclo[1.1.1]pentan-1-yl)carbamate

To an ice-cooled solution of the product of Example 282E (45 mg, 0.180 mmol) and tert-butyl (3-aminobicyclo[1.1.1]pentan-1-yl)carbamate (42.8 mg, 0.216 mmol) in dichloromethane (1 mL) was added N,N-diisopropylethylamine (0.126 mL, 0.719 mmol) followed by 2-(3H-[1,2,3]triazolo[4,5-b]pyridin-3-yl)-1,1,3,3-tetramethylisouronium hexafluorophosphate (V) (HATU, 103 mg, 0.270 mmol) and the reaction mixture was stirred at ambient temperature for 1 hour. The reaction mixture was diluted with dichloromethane (5 mL) and washed with 1 M aqueous HCl (2×5 mL) using a phase separator cartridge. The organic phase was concentrated under reduced pressure to give the title product (43 mg, 0.098 mmol, 54.4% yield). MS (ESI) m/z 431 (M+H)⁺.

### Example 282G: N-(3-{[(2R,4R)-6-chloro-4-hydroxy-3,4-dihydro-2H-1-benzopyran-2-carbonyl]amino}bicyclo[1.1.1]pentan-1-yl)-1-[cis-3-(trifluoromethoxy)cyclobutyl]-1H-pyrazole-4-carboxamide

To a solution of the product of Example 282F (43 mg, 0.10 mmol) in dichloromethane (2 mL) was added trifluoroacetic acid (0.12 mL, 1.5 mmol). The resulting mixture was stirred at ambient temperature for 1 hour. To the reaction mixture was added an SCX resin (about 1 g) and the suspension was stirred for 10 minutes, filtered, and then washed with methanol (20 mL). The filtrate from the methanol wash was discarded. The filter cake was further washed with an ammonia solution (3.5 M in methanol, 20 mL), and the filtrate from the second wash was concentrated in vacuo to afford the crude product of N-(3-aminobicyclo[1.1.1]pentan-1-yl)-1-(cis-3-(trifluoromethoxy)cyclobutyl)-1H-pyrazole-4-carboxamide (13 mg, 0.038 mmol), which was then combined with the product of Example 245C (10.8 mg, 0.047 mmol), N,N-diisopropylethylamine (0.048 mL, 0.276 mmol), and anhydrous N,N-dimethylformamide (1 mL). The reaction mixture was stirred in an ice-water bath under a nitrogen atmosphere, and a 50% solution of propanephosphonic acid anhydride (T3P®) in N,N-dimethylformamide (0.027 mL, 0.047 mmol) was added. The resulting solution was allowed to warm to room temperature and stirred for 1 hour. The mixture was directly purified by preparative HPLC [Waters XBridge™ C18 5 μm OBD column, 19×50 mm, 25-55% gradient of acetonitrile in buffer (0.1% aqueous ammonium bicarbonate)] to afford the title compound (6 mg, 11% yield). ¹H NMR (500 MHz, DMSO-d₆) δ ppm 8.69 (s, 1H), 8.60 (s, 1H), 8.26 (s, 1H), 7.91 (s, 1H), 7.39 (dd, J=2.8, 1.0 Hz, 1H), 7.21 (dd, J=8.8, 2.7 Hz, 1H), 6.89 (d, J=8.7 Hz, 1H), 5.71 (s, 1H), 4.85-4.79 (m, 1H), 4.79-4.72 (m, 1H), 4.66-4.56 (m, 2H), 3.00-2.91 (m, 2H), 2.74-2.63 (m, 2H), 2.39-2.33 (m, 1H), 2.31 (s, 6H), 1.70 (q, J=12.0 Hz, 1H); MS (ESI) m/z 541 (M+H)⁺.

### Example 283: N-(3-{[(2R,4R)-6-chloro-4-hydroxy-3,4-dihydro-2H-1-benzopyran-2-carbonyl]amino}bicyclo[1.1.1]pentan-1-yl)-5-methylpyrazine-2-carboxamide (Compound 382)

The reaction and purification conditions described in Examples 274A through 274C substituting tert-butyl (3-aminobicyclo[1.1.1]pentan-1-yl)carbamate for tert-butyl (4-aminobicyclo[2.1.1]hexan-1-yl)carbamate, and 5-methylpyrazine-2-carboxylic acid for 5-(trifluoromethoxy)picolinic acid gave the title compound. ¹H NMR (500 MHz, DMSO-d₆) δ ppm 9.38 (s, 1H), 9.01 (d, J=1.5 Hz, 1H), 8.71 (s, 1H), 8.60 (d, J=1.4 Hz, 1H), 7.38 (dd, J=2.7, 1.0 Hz, 1H), 7.20 (dd, J=8.7, 2.7 Hz, 1H), 6.89 (d, J=8.7 Hz, 1H), 5.73 (br s, 1H), 4.81 (dd, J=10.7, 5.9 Hz, 1H), 4.61 (dd, J=12.0, 2.3 Hz, 1H), 2.59 (s, 3H), 2.37 (s, 6H), 2.40-2.32 (m, 1H), 1.77-1.65 (m, 1H); MS (APCI⁺) m/z 411 (M−H₂O+H)⁺.

### Example 284: N-(3-{[(2R,4R)-6-chloro-4-hydroxy-3,4-dihydro-2H-1-benzopyran-2-carbonyl]amino}bicyclo[1.1.1]pentan-1-yl)-2-cyclopropyl-1,3-oxazole-5-carboxamide (Compound 383)

The reaction and purification conditions described in Examples 274A through 274C substituting tert-butyl (3-aminobicyclo[1.1.1]pentan-1-yl)carbamate for tert-butyl (4-aminobicyclo[2.1.1]hexan-1-yl)carbamate, and 2-cyclopropyloxazole-5-carboxylic acid (J-W Pharmlab) for 5-(trifluoromethoxy)picolinic acid gave the title compound. ¹H NMR (500 MHz, DMSO-d₆) δ ppm 9.00 (s, 1H), 8.72 (s, 1H), 7.56 (s, 1H), 7.38 (dd, J=2.7, 1.0 Hz, 1H), 7.20 (ddd, J=8.7, 2.7, 0.7 Hz, 1H), 6.88 (d, J=8.7 Hz, 1H), 5.71 (br s, 1H), 4.81 (dd, J=10.7, 5.9 Hz, 1H), 4.61 (dd, J=12.0, 2.3 Hz, 1H), 2.39-2.27 (m, 1H), 2.32 (s, 6H), 2.19-2.10 (m, 1H), 1.77-1.64 (m, 1H), 1.13-0.99 (m, 4H); MS (APCI⁺) m/z 444 (M+H)⁺.

### Example 285: N-(3-{[(2R,4R)-6-chloro-4-hydroxy-3,4-dihydro-2H-1-benzopyran-2-carbonyl]amino}bicyclo[1.1.1]pentan-1-yl)-2-ethyl-1,3-oxazole-5-carboxamide (Compound 384)

The reaction and purification conditions described in Examples 274A through 274C substituting tert-butyl (3-aminobicyclo[1.1.1]pentan-1-yl)carbamate for tert-butyl (4-aminobicyclo[2.1.1]hexan-1-yl)carbamate, and 2-ethyloxazole-5-carboxylic acid (J-W Pharmlab) for 5-(trifluoromethoxy)picolinic acid gave the title compound. ¹H NMR (400 MHz, DMSO-d₆) δ ppm 9.07 (s, 1H), 8.71 (s, 1H), 7.60 (s, 1H), 7.38 (dd, J=2.7, 0.9 Hz, 1H), 7.20 (dd, J=8.7, 2.7 Hz, 1H), 6.88 (d, J=8.7 Hz, 1H), 5.72 (s, 1H), 4.81 (dd, J=10.8, 6.0 Hz, 1H), 4.60 (dd, J=12.0, 2.3 Hz, 1H), 2.79 (q, J=7.6 Hz, 2H), 2.40-2.32 (m, 1H), 2.32 (s, 6H), 1.70 (td, J=12.5, 10.8 Hz, 1H), 1.25 (t, J=7.6 Hz, 3H); MS (APCI⁺) m/z 444 (M−H₂O+H)⁺.

### Example 286: N-(3-{[(2R,4R)-6-chloro-4-hydroxy-3,4-dihydro-2H-1-benzopyran-2-carbonyl]amino}bicyclo[1.1.1]pentan-1-yl)-2-methyl-1,3-thiazole-5-carboxamide (Compound 385)

The reaction and purification conditions described in Examples 274A through 274C substituting tert-butyl (3-aminobicyclo[1.1.1]pentan-1-yl)carbamate for tert-butyl (4-aminobicyclo[2.1.1]hexan-1-yl)carbamate, and 2-methylthiazole-5-carboxylic acid (Atlantic Research Chemicals) for 5-(trifluoromethoxy)picolinic acid gave the title compound. ¹H NMR (400 MHz, DMSO-d₆) δ ppm 9.15 (s, 1H), 8.72 (s, 1H), 8.17 (s, 1H), 7.38 (dd, J=2.6, 0.9 Hz, 1H), 7.20 (dd, J=8.5, 2.7 Hz, 1H), 6.89 (d, J=8.8 Hz, 1H), 5.72 (br s, 1H), 4.81 (dd, J=10.7, 5.9 Hz, 1H), 4.60 (dd, J=12.0, 2.3 Hz, 1H), 2.65 (s, 3H), 2.40-2.32 (m, 1H), 2.33 (s, 6H), 1.70 (td, J=12.4, 10.7 Hz, 1H); MS (APCI⁺) m/z 416 (M−H₂O+H)⁺.

### Example 287: N-(3-{[(2R,4R)-6-chloro-4-hydroxy-3,4-dihydro-2H-1-benzopyran-2-carbonyl]amino}bicyclo[1.1.1]pentan-1-yl)pyridine-2-carboxamide (Compound 386)

The reaction and purification conditions described in Examples 274A through 274C substituting tert-butyl (3-aminobicyclo[1.1.1]pentan-1-yl)carbamate for tert-butyl (4-aminobicyclo[2.1.1]hexan-1-yl)carbamate, and picolinic acid for 5-(trifluoromethoxy)picolinic acid gave the title compound. ¹H NMR (400 MHz, DMSO-d₆) δ ppm 9.25 (s, 1H), 8.70 (s, 1H), 8.62 (dt, J=4.7, 1.4 Hz, 1H), 8.04-7.95 (m, 2H), 7.64-7.56 (m, 1H), 7.38 (dd, J=2.6, 1.0 Hz, 1H), 7.20 (dd, J=8.8, 2.9 Hz, 1H), 6.89 (d, J=8.7 Hz, 1H), 5.74-5.70 (m, 1H), 4.83-4.79 (m, 1H), 4.61 (dd, J=12.0, 2.3 Hz, 1H), 2.37 (s, 6H), 2.41-2.31 (m, 1H), 1.77-1.64 (m, 1H); MS (APCI⁺) m/z 414 (M+H)⁺.

### Example 288: N-(3-{[(2R,4R)-6-chloro-4-hydroxy-3,4-dihydro-2H-1-benzopyran-2-carbonyl]amino}bicyclo[1.1.1]pentan-1-yl)-2-cyclobutyl-1,3-oxazole-5-carboxamide (Compound 387)

### Example 288A: 2-cyclobutyloxazole-5-carboxylic Acid

A 20 mL vial was charged with methyl 2-bromooxazole-5-carboxylate (250 mg, 1.21 mmol, Combi-Blocks), tris(dibenzylideneacetone)dipalladium(0) (16.7 mg, 0.018 mmol), tri(2-furyl)phosphine (16.9 mg, 0.073 mmol) and N,N-dimethylformamide (3 mL). The vial was purged with a nitrogen stream for 2 minutes, sealed and stirred at ambient temperature. Then cyclobutylzinc(II) bromide (0.5 M in tetrahydrofuran, 3.40 mL) was added dropwise over a period of 2 minutes. After stirring at ambient temperature for 20 minutes, water (1 mL) and methanol (1 mL) were added, and the resulting reaction mixture was stirred for 5 minutes and then concentrated under reduced pressure briefly to remove most of the volatiles. The resulting mixture was filtered through a microfiber frit and directly purified by reversed-phase flash chromatography [Interchim 120 g C18XS column, flow rate 60 mL/minute, 5-100% gradient of acetonitrile in buffer (0.025 M aqueous ammonium bicarbonate, adjusted to pH 10 with ammonium hydroxide)]. Fractions containing both the title compound and the methyl ester form of the title compound were combine (about 100 mL), and aqueous NaOH (2.5 M, 3.4 mL) was added. Then the mixture was stirred at ambient temperature for 10 minutes. The resulting solution was partitioned between dichloromethane (2×100 mL) and aqueous citric acid (10 weight %, 50 mL). The organic layers were combined, dried over sodium sulfate, and concentrated under reduced pressure to give the title compound (30 mg, 0.18 mmol, 15% yield). ¹H NMR (500 MHz, methanol-d₄) δ ppm 7.45 (s, 1H), 3.70 (pd, J=8.6, 1.1 Hz, 1H), 2.51-2.33 (m, 4H), 2.19-2.05 (m, 1H), 2.05-1.92 (m, 1H); MS (APCI⁺) m/z 168 (M+H)⁺.

### Example 288B: N-(3-{[(2R,4R)-6-chloro-4-hydroxy-3,4-dihydro-2H-1-benzopyran-2-carbonyl]amino}bicyclo[1.1.1]pentan-1-yl)-2-cyclobutyl-1,3-oxazole-5-carboxamide

1-[Bis(dimethylamino)methylene]-1H-1,2,3-triazolo[4,5-b]pyridinium 3-oxid hexafluorophosphate (HATU, 71 mg, 0.187 mmol) was added to a mixture of the product of Example 288A (26 mg, 0.156 mmol), tert-butyl (3-aminobicyclo[1.1.1]pentan-1-yl)carbamate (31 mg, 0.156 mmol), and triethylamine (0.108 mL, 0.778 mmol) in N,N-dimethylformamide (2 mL). The resulting mixture was stirred at ambient temperature for 1 hour and then water (0.1 mL) was added. The resulting solution was filtered through a glass microfiber frit and purified by preparative HPLC [YMC TriArt™ C18 Hybrid 5 μm column, 50×100 mm, flow rate 140 mL/minute, 5-100% gradient of acetonitrile in buffer (0.025 M aqueous ammonium bicarbonate, adjusted to pH 10 with ammonium hydroxide)] to give tert-butyl (3-(2-cyclobutyloxazole-5-carboxamido)bicyclo[1.1.1]pentan-1-yl)carbamate (42 mg, 0.121 mmol, 78%). MS (APCI⁺) m/z 348 (M+H)⁺. A portion of this intermediate (20 mg, 0.058 mmol) and the product of Example 124A (13 mg, 0.058 mmol) were processed as described in Examples 274B and Example 274C to give the title compound (22 mg, 0.048 mmol, 83% yield). ¹H NMR (400 MHz, DMSO-d₆) δ ppm 9.07 (s, 1H), 8.71 (s, 1H), 7.62 (s, 1H), 7.38 (dd, J=2.7, 0.9 Hz, 1H), 7.20 (dd, J=8.7, 2.7 Hz, 1H), 6.88 (d, J=8.7 Hz, 1H), 5.68 (br s, 1H), 4.81 (dd, J=10.6, 5.8 Hz, 1H), 4.60 (dd, J=11.9, 2.2 Hz, 1H), 3.74-3.58 (m, 1H), 2.40-2.28 (m, 11H), 2.11-1.96 (m, 1H), 1.96-1.85 (m, 1H), 1.76-1.63 (m, 1H); MS (APCI⁺) m/z 458 (M+H)⁺.

### Example 289: N-(3-{[(2R,4R)-6-chloro-4-hydroxy-3,4-dihydro-2H-1-benzopyran-2-carbonyl]amino}bicyclo[1.1.1]pentan-1-yl)-2-[1-(2,2,2-trifluoroethyl)azetidin-3-yl]-1,3-oxazole-5-carboxamide (Compound 388)

### Example 289A: 2-(1-(tert-butoxycarbonyl)azetidin-3-yl)oxazole-5-carboxylic Acid)

To a 30 mL vial equipped with a stir bar was added [4,4′-bis(1,1-dimethylethyl)-2,2′-bipyridine-N1,N1′]bis[3,5-difluoro-2-[5-(trifluoromethyl)-2-pyridinyl-N]phenyl-C]Iridium(III) hexafluorophosphate (23.8 mg, 0.021 mmol, [Ir{dF(CF₃)ppy}₂(dtbpy)]PF₆), methyl 2-bromooxazole-5-carboxylate (350 mg, 1.70 mmol, Combi-Blocks), tert-butyl 3-bromoazetidine-1-carboxylate (602 mg, 2.55 mmol, Enamine), tris(trimethylsilyl)silane (0.524 mL, 1.67 mmol), and anhydrous LiOH (81 mg, 3.40 mmol). The vial was sealed, purged with nitrogen then to it was added 1,2-dimethoxyethane (4 mL). To a separate 4 mL vial was added nickel(II) chloride ethylene glycol dimethyl ether complex (93 mg, 0.425 mmol) and 4,4′-di-tert-butyl-2,2′-bipyridine (125 mg, 0.467 mmol). The catalyst vial was sealed, purged with nitrogen, and then 1,2-dimethoxyethane (2 mL) was added. This precatalyst solution was stirred at ambient temperature for 5 minutes, after which, 1 mL (out of the 2 mL total) was syringed into the reaction vessel. The resulting reaction solution was degassed by sparging with nitrogen while stirring for 10 minutes before sealing with Parafilm©. The reaction vial was put inside a continuously running tap water cooled bath and the reaction was stirred and irradiated using 2 lamps: a 40W Kessil PR160 390 nm Photoredox lamp and a PAR20-18W CREE XPE 450 nm blue LED lamp (both lamps were placed 3 cm away from the reaction vial set inside the water bath). The reaction temperature was measured to be around 18° C. when the reaction started, and was maintained at that temperature for the duration of the reaction. After 12 hours, the reaction mixture was first quenched by exposing to air and then the mixture was partitioned between dichloromethane (2×50 mL) and saturated aqueous sodium bicarbonate (50 mL). The combined organic layers were dried over sodium sulfate, concentrated under reduced pressure, and then taken up in methanol (3 mL). Aqueous sodium hydroxide (2.04 mL, 2.5 M) was added. The resulting mixture was stirred at ambient temperature for 30 minutes, filtered through a glass microfiber frit, and directly purified by reverse phase flash chromatography [Custom packed YMC TriArt™ C18 Hybrid 20 μm column, 25×150 mm, flow rate 70 mL/minute, 5-100% gradient of acetonitrile in carbonic acid buffer (prepared by adding 50 g of large chunks of dry ice into every 4 L of deionized water until bubbling stops; buffer freshly prepared every hour)] to provide the salt free form of the title compound (142 mg, 0.53 mmol, 31% yield). ¹H NMR (600 MHz, DMSO-d₆) δ ppm 7.77 (s, 1H), 4.22 (s, 2H), 4.03 (dd, J=10.6, 5.2 Hz, 3H), 1.39 (s, 9H); MS (ESI⁻) m/z 267 (M−H)⁻.

### Example 289B: tert-butyl 3-{5-[(3-{[(2R)-6-chloro-4-oxo-3,4-dihydro-2H-1-benzopyran-2-carbonyl]amino}bicyclo[1.1.1]pentan-1-yl)carbamoyl]-1,3-oxazol-2-yl}azetidine-1-carboxylate

The reaction and purification conditions described in Examples 274A through 274B substituting tert-butyl (3-aminobicyclo[1.1.1]pentan-1-yl)carbamate for tert-butyl (4-aminobicyclo[2.1.1]hexan-1-yl)carbamate, and the product of Example 289A for 5-(trifluoromethoxy)picolinic acid gave the title compound. ¹H NMR (500 MHz, CDCl₃) δ ppm 7.89 (d, J=2.6 Hz, 1H), 7.64 (s, 1H), 7.49 (dd, J=8.8, 2.7 Hz, 1H), 7.08-7.03 (m, 2H), 6.66 (s, 1H), 4.86 (dd, J=13.5, 3.3 Hz, 1H), 4.31 (t, J=8.9 Hz, 2H), 4.26-4.19 (m, 2H), 3.92 (tt, J=8.9, 6.0 Hz, 1H), 3.19 (dd, J=17.3, 3.3 Hz, 1H), 2.87 (dd, J=17.3, 13.5 Hz, 1H), 2.60 (s, 6H), 1.46 (s, 9H); MS (APCI⁺) m/z 557 (M+H)⁺.

### Example 289C: N-(3-{[(2R,4R)-6-chloro-4-hydroxy-3,4-dihydro-2H-1-benzopyran-2-carbonyl]amino}bicyclo[1.1.1]pentan-1-yl)-2-[1-(2,2,2-trifluoroethyl)azetidin-3-yl]-1,3-oxazole-5-carboxamide

Trifluoroacetic acid (1.0 mL) was added to the product of Example 289B (66 mg, 0.12 mmol). After stirring at ambient temperature for 30 minutes, the mixture was concentrated under reduced pressure. To the resulting residue was added acetonitrile (2 mL) and potassium carbonate (82 mg, 0.592 mmol). While stirring at ambient temperature, 2,2,2-trifluoroethyl trifluoromethanesulfonate (0.051 mL, 0.355 mmol) was added. The reaction mixture was then stirred at 70° C. for 1 hour, cooled to ambient temperature, and partitioned between dichloromethane (2×20 mL) and aqueous sodium carbonate (1.0 M, 20 mL). The organic layers were combined, dried over sodium sulfate, and concentrated under reduced pressure. The residue was taken up in methanol (2 mL). While stirring at ambient temperature, sodium borohydride (27 mg, 0.71 mmol) was added in one portion. After stirring for 10 minutes, saturated aqueous ammonium chloride (0.1 mL) was added, and the mixture was again partitioned between dichloromethane (2×20 mL) and aqueous sodium carbonate (1.0 M, 20 mL). The organic layers were combined, dried over sodium sulfate, and concentrated under reduced pressure. The residue was purified by preparative HPLC [YMC TriArt™ C18 Hybrid 5 μm column, 50×100 mm, flow rate 140 mL/minute, 5-100% gradient of acetonitrile in buffer (0.025 M aqueous ammonium bicarbonate, adjusted to pH 10 with ammonium hydroxide)] to give the title compound (5 mg, 9.2 μmol, 8% yield). ¹H NMR (500 MHz, CDCl₃) δ ppm 7.61 (s, 1H), 7.45 (d, J=2.6 Hz, 1H), 7.17 (dd, J=8.7, 2.6 Hz, 1H), 7.00 (s, 1H), 6.85 (d, J=8.7 Hz, 1H), 6.64 (s, 1H), 4.93 (dd, J=8.8, 5.5 Hz, 1H), 4.60 (dd, J=9.8, 3.1 Hz, 1H), 3.99-3.86 (m, 3H), 3.69-3.58 (m, 2H), 3.09 (q, J=9.3 Hz, 2H), 2.66 (ddd, J=13.6, 5.6, 3.1 Hz, 1H), 2.46-2.35 (m, 1H), 2.12 (dt, J=13.5, 9.3 Hz, 1H); MS (APCI⁺) m/z 514 (M+H)⁺.

### Example 290: 2-(azetidin-3-yl)-N-(3-{[(2R,4R)-6-chloro-4-hydroxy-3,4-dihydro-2H-1-benzopyran-2-carbonyl]amino}bicyclo[1.1.1]pentan-1-yl)-1,3-oxazole-5-carboxamide (Compound 389)

Trifluoroacetic acid (0.25 mL) was combined with the product of Example 289B (11 mg, 0.02 mmol). The resulting mixture was stirred at ambient temperature for 30 minutes and then concentrated under reduced pressure. The residue was partitioned between dichloromethane (2×30 mL) and aqueous sodium carbonate (1.0 M, 30 mL). The organic layers were combined and dried over sodium sulfate, concentrated under reduced pressure, and then taken up in methanol (2 mL). While stirring at ambient temperature, sodium borohydride (7.5 mg, 0.20 mmol) was added in one portion. After stirring for another 20 minutes, saturated aqueous ammonium chloride (0.1 mL) was added, and the mixture was again partitioned between dichloromethane (2×30 mL) and aqueous sodium carbonate (1.0 M, 30 mL). The organic layers were combined, dried over sodium sulfate, and concentrated under reduced pressure. The residue was purified by preparative HPLC [YMC TriArt™ C18 Hybrid 5 μm column, 50×100 mm, flow rate 140 mL/minute, 5-100% gradient of acetonitrile in buffer (0.025 M aqueous ammonium bicarbonate, adjusted to pH 10 with ammonium hydroxide)] to give the title compound (7 mg, 0.015 mmol, 77% yield). ¹H NMR (400 MHz, CDCl₃) δ ppm 7.62 (s, 1H), 7.45 (d, J=2.4 Hz, 1H), 7.18 (dd, J=8.6, 2.6 Hz, 1H), 6.97 (s, 1H), 6.92-6.87 (m, 1H), 6.85 (d, J=8.7 Hz, 1H), 4.94 (dd, J=8.9, 5.7 Hz, 1H), 4.61 (dd, J=9.7, 3.1 Hz, 1H), 4.12-4.03 (m, 1H), 3.99 (s, 2H), 4.02-3.94 (m, 2H), 2.72-2.61 (m, 1H), 2.55 (s, 6H), 2.18-2.06 (m, 1H); MS (APCI⁺) m/z 459 (M+H)⁺.

### Example 291: N-(3-{[(2R,4R)-6-chloro-4-hydroxy-3,4-dihydro-2H-1-benzopyran-2-carbonyl]amino}bicyclo[1.1.1]pentan-1-yl)-1-phenyl-1H-pyrazole-4-carboxamide (Compound 390)

### Example 291A: tert-butyl {3-[(1-phenyl-1H-pyrazole-4-carbonyl)amino]bicyclo[1.1.1]pentan-1-yl}carbamate

To an ice-cooled solution of 1-phenyl-1H-pyrazole-4-carboxylic acid (56 mg, 0.29 mmol) and tert-butyl (3-aminobicyclo[1.1.1]pentan-1-yl)carbamate (PharmaBlock, 63.6 mg, 0.321 mmol) in dichloromethane (2 mL) and N,N-dimethylformamide (3 mL) was added N,N-diisopropylethylamine (0.20 mL, 1.2 mmol) followed by 2-(3H-[1,2,3]triazolo[4,5-b]pyridin-3-yl)-1,1,3,3-tetramethylisouronium hexafluorophosphate (V) (HATU, 166 mg, 0.437 mmol) and the reaction mixture was stirred at ambient temperature for 20 hours. The reaction mixture was then diluted with dichloromethane (100 mL) and washed with HCl (1 M, 2×50 mL). The organic layer was concentrated under reduced pressure to give the crude product. The crude product was purified by chromatography on silica gel (₀-1₀₀% ethyl acetate in isohexanes) to afford the title intermediate (52.9 mg, 0.136 mmol, 47% yield). ¹H NMR (500 MHz, DMSO-d₆) δ ppm 8.86 (s, 1H), 8.71 (s, 1H), 8.10 (s, 1H), 7.85-7.79 (m, 2H), 7.59-7.48 (m, 3H), 7.39-7.32 (m, 1H), 2.20 (s, 6H), 1.39 (s, 9H); MS (ESI⁺) m/z 369 (M+H)⁺.

### Example 291B: N-(3-aminobicyclo[1.1.1]pentan-1-yl)-1-phenyl-1H-pyrazole-4-carboxamide

To a solution of the product of Example 291A (25 mg, 0.064 mmol) in dichloromethane (2.5 mL) was added trifluoroacetic acid (250 μL, 3.24 mmol) and the reaction mixture stirred at ambient temperature for 1 hour and then was diluted with methanol (15 mL). SCX resin (SiliCycle® SiliaBond®-Tosic acid resin, 500 mg) was added and the reaction mixture was stirred for 30 minutes. The mixture was loaded onto additional SCX resin (1 g), washed with methanol (25 mL) and eluted with 0.7 M NH₃ in methanol (25 mL) to afford the title intermediate (19.1 mg, 0.065 mmol, quantitative yield). ¹H NMR (500 MHz, DMSO-d₆) δ ppm 8.85 (s, 1H), 8.58 (s, 1H), 8.09 (s, 1H), 7.85-7.79 (m, 2H), 7.56-7.48 (m, 2H), 7.38-7.32 (m, 1H), 2.38-2.24 (m, 2H), 2.00 (s, 6H); MS (ESI⁺) m/z 269 (M+H)⁺.

### Example 291C: N-(3-{[(2R,4R)-6-chloro-4-hydroxy-3,4-dihydro-2H-1-benzopyran-2-carbonyl]amino}bicyclo[1.1.1]pentan-1-yl)-1-phenyl-1H-pyrazole-4-carboxamide

The products of Example 245C (21 mg, 0.095 mmol) and Example 291B (17 mg, 0.054 mmol) were dissolved in N,N-dimethylformamide (0.7 mL) at room temperature. To this reaction mixture was added N-ethyl-N-isopropylpropan-2-amine (0.077 mL, 0.44 mmol) and 2,4,6-tripropyl-1,3,5,2,4,6-trioxatriphosphinane 2,4,6-trioxide (T3P© in 50% N,N-dimethylformamide, 0.044 mL, 0.076 mmol), and the reaction mixture was stirred at room temperature for 4 days. The reaction mixture was then purified by preparative HPLC [Waters XBridge™ C18 5 μm, 19×50 mm, 20-55% gradient of acetonitrile in buffer (0.025 M aqueous ammonium bicarbonate, adjusted to pH 10 with ammonium hydroxide)] to give the title compound (10 mg, 0.021 mmol, 34% yield). ¹H NMR (500 MHz, DMSO-d₆) δ ppm 8.89 (s, 1H), 8.79 (s, 1H), 8.74 (s, 1H), 8.12 (s, 1H), 7.91-7.80 (m, 2H), 7.54 (t, J=8.0 Hz, 2H), 7.44-7.33 (m, 2H), 7.22 (dd, J=8.7, 2.7 Hz, 1H), 6.90 (d, J=8.7 Hz, 1H), 5.73 (d, J=6.2 Hz, 1H), 4.84-4.79 (m, 1H), 4.62 (dd, J=11.9, 2.2 Hz, 1H), 2.35 (s, 6H), 1.78-1.63 (m, 2H); MS (ESI+) m/z 480 (M+H)⁺.

### Example 292: 1-(4-chloro-3-fluorophenyl)-N-(3-{[(2R,4R)-6-chloro-4-hydroxy-3,4-dihydro-2H-1-benzopyran-2-carbonyl]amino}bicyclo[1.1.1]pentan-1-yl)-1H-pyrazole-4-carboxamide (Compound 391)

### Example 292A: ethyl 1-(4-chloro-3-fluorophenyl)-1H-pyrazole-4-carboxylate

A mixture of ethyl 1H-pyrazole-4-carboxylate (1.0 g, 7.1 mmol), 4-bromo-1-chloro-2-fluorobenzene (1.50 ml, 10.7 mmol), potassium carbonate (3.16 g, 22.8 mmol), copper(I) iodide (0.272 g, 1.43 mmol) and (1S,2S)—N¹,N²-dimethylcyclohexane-1,2-diamine (1.02 g, 7.14 mmol) in N,N-dimethylformamide (10 mL) was heated at 110° C. for 3 hours. Then the reaction mixture was partitioned between ethyl acetate (100 mL) and water (25 mL). The aqueous layer was extracted with ethyl acetate (2×50 mL). The organic layers were combined, washed with brine (2×50 mL), dried over MgSO₄, filtered, and concentrated in vacuo. The residue was purified by chromatography on silica gel (0-25% ethyl acetate in isohexane) to afford the title intermediate (1.33 g, 4.85 mmol, 68% yield). ¹H NMR (500 MHz, DMSO-d₆) δ ppm 9.20 (s, 1H), 8.18 (s, 1H), 8.09 (dd, J=10.7, 2.5 Hz, 1H), 7.89-7.84 (m, 1H), 7.80-7.72 (m, 1H), 4.28 (q, J=7.1 Hz, 2H), 1.31 (t, J=7.1 Hz, 3H).

### Example 292B: 1-(4-chloro-3-fluorophenyl)-1H-pyrazole-4-carboxylic Acid

To a mixture of the product of Example 292A (250 mg, 0.912 mmol) in tetrahydrofuran (10 mL) was added lithium hydroxide (1 M in H₂O, 3.65 mL, 3.65 mmol). To this suspension was added methanol (3 mL) dropwise until the reaction became homogeneous. The reaction mixture was stirred at ambient temperature overnight. The reaction mixture was diluted with water (2 mL) and was then concentrated in vacuo to remove the methanol and tetrahydrofuran. The aqueous mixture was diluted with H₂O (3 mL) and the aqueous layer was washed with ethyl acetate (2×5 mL). To the aqueous layer was added HCl (1 M, aqueous) dropwise until a precipitate was observed. The precipitate was collected by filtration, washed with water (2×2 mL), and dried to afford the title intermediate (78 mg, 0.318 mmol, 34.8% yield). The filtrate was acidified further to pH 2-3 to afford more precipitate, which was collected by filtration, washed with water (2×2 mL), and dried to afford more of the title intermediate (101 mg, 0.411 mmol, 45% yield). ¹H NMR (500 MHz, DMSO-d₆) δ ppm 12.76 (s, 1H), 9.12 (s, 1H), 8.12 (s, 1H), 8.07 (dd, J=10.7, 2.5 Hz, 1H), 7.87-7.82 (m, 1H), 7.79-7.72 (m, 1H); MS (ESI⁺) m/z 241 (M+H)⁺.

### Example 292C: tert-butyl (3-{[1-(4-chloro-3-fluorophenyl)-1H-pyrazole-4-carbonyl]amino}bicyclo[1.1.1]pentan-1-yl)carbamate

The methodologies described in Example 291A substituting Example 292B for 1-phenyl-1H-pyrazole-4-carboxylic acid gave the title intermediate. ¹H NMR (500 MHz, DMSO-d₆) δ ppm 8.95 (s, 1H), 8.75 (s, 1H), 8.15 (s, 1H), 7.99-7.93 (m, 1H), 7.78-7.72 (m, 2H), 7.56 (s, 1H), 2.20 (s, 6H), 1.38 (s, 9H); MS (ESI⁺) m/z 365 (M−C(CH₃)₃+H)⁺.

### Example 292D: N-(3-aminobicyclo[1.1.1]pentan-1-yl)-1-(4-chloro-3-fluorophenyl)-1H-pyrazole-4-carboxamide

The methodologies described in Example 291B substituting Example 292C for Example 291A gave the title intermediate. ¹H NMR (500 MHz, DMSO-d₆) δ ppm 8.93 (s, 1H), 8.62 (s, 1H), 8.14 (s, 1H), 7.99-7.92 (m, 1H), 7.78-7.70 (m, 2H), 2.34-2.18 (m, 2H), 1.99 (s, 6H); MS (ESI⁺) m/z 321 (M+H)⁺.

### Example 292E: 1-(4-chloro-3-fluorophenyl)-N-(3-{[(2R,4R)-6-chloro-4-hydroxy-3,4-dihydro-2H-1-benzopyran-2-carbonyl]amino}bicyclo[1.1.1]pentan-1-yl)-1H-pyrazole-4-carboxamide

The methodologies described in Example 291C substituting Example 292D for Example 291B gave the title compound. ¹H NMR (500 MHz, DMSO-d₆) δ ppm 8.98 (s, 1H), 8.83 (s, 1H), 8.74 (s, 1H), 8.18 (s, 1H), 7.98 (d, J=10.4 Hz, 1H), 7.79-7.75 (m, 1H), 7.39 (d, J=2.7 Hz, 1H), 7.22 (dd, J=8.7, 2.7 Hz, 1H), 6.90 (d, J=8.7 Hz, 1H), 5.75-5.70 (m, 1H), 4.84-4.79 (m, 1H), 4.65-4.60 (m, 1H), 2.35 (s, 6H), 1.76-1.65 (m, 2H); MS (ESI⁻) m/z 531 (M−H)⁻.

### Example 293: N-(3-{[(2R,4R)-6-chloro-4-hydroxy-3,4-dihydro-2H-1-benzopyran-2-carbonyl]amino}bicyclo[1.1.1]pentan-1-yl)-1-[6-(trifluoromethyl)pyridin-3-yl]-1H-pyrazole-4-carboxamide (Compound 392)

### Example 293A: ethyl 1-[6-(trifluoromethyl)pyridin-3-yl]-1H-pyrazole-4-carboxylate

The methodologies described in Example 292A substituting 5-bromo-2-(trifluoromethyl)pyridine for 4-bromo-1-chloro-2-fluorobenzene gave the title intermediate. ¹H NMR (500 MHz, DMSO-d₆) δ ppm 9.39 (d, J=2.5 Hz, 1H), 9.37 (s, 1H), 8.62 (dd, J=8.7, 2.6 Hz, 1H), 8.28 (s, 1H), 8.11 (d, J=8.4 Hz, 1H), 4.30 (q, J=7.1 Hz, 2H), 1.32 (t, J=7.1 Hz, 3H); MS (ESI⁻) m/z 284 (M−H)⁻.

### Example 293B: 1-[6-(trifluoromethyl)pyridin-3-yl]-1H-pyrazole-4-carboxylic acid

The methodologies described in Example 292B substituting the product of Example 293A for the product of Example 292A gave the title intermediate. ¹H NMR (500 MHz, DMSO-d₆) δ ppm 12.88 (s, 1H), 9.38 (d, J=2.5 Hz, 1H), 9.28 (s, 1H), 8.60 (dd, J=8.6, 2.6 Hz, 1H), 8.22 (s, 1H), 8.10 (d, J=8.6 Hz, 1H); MS (ESI⁺) m/z 258 (M+H)⁺.

### Example 293C: tert-butyl [3-({1-[6-(trifluoromethyl)pyridin-3-yl]-1H-pyrazole-4-carbonyl}amino)bicyclo[1.1.1]pentan-1-yl]carbamate

The methodologies described in Example 291A substituting the product of Example 293B for 1-phenyl-1H-pyrazole-4-carboxylic acid gave the title intermediate. ¹H NMR (500 MHz, DMSO-d₆) δ ppm 9.30 (d, J=2.6 Hz, 1H), 9.11 (s, 1H), 8.83 (s, 1H), 8.51 (dd, J=8.6, 2.6 Hz, 1H), 8.26 (s, 1H), 8.10 (d, J=8.6 Hz, 1H), 7.57 (s, 1H), 2.21 (s, 6H), 1.39 (s, 9H); MS (ESI-) m/z 436 (M−H)⁻.

### Example 293D: N-(3-aminobicyclo[1.1.1]pentan-1-yl)-1-[6-(trifluoromethyl)pyridin-3-yl]-1H-pyrazole-4-carboxamide

The methodologies described in Example 291B substituting the product of Example 293C for the product of Example 291A gave the title intermediate. ¹H NMR (500 MHz, DMSO-d₆) δ ppm 9.30 (d, J=2.6 Hz, 1H), 9.10 (s, 1H), 8.74 (s, 1H), 8.51 (dd, J=8.5, 2.6 Hz, 1H), 8.25 (s, 1H), 8.09 (d, J=8.6 Hz, 1H), 2.04 (s, 6H); MS (ESI⁺) m/z 338 (M+H)⁺.

### Example 293E: N-(3-{[(2R,4R)-6-chloro-4-hydroxy-3,4-dihydro-2H-1-benzopyran-2-carbonyl]amino}bicyclo[1.1.1]pentan-1-yl)-1-[6-(trifluoromethyl)pyridin-3-yl]-1H-pyrazole-4-carboxamide

The methodologies described in Example 291C substituting the product of Example 293D for the product of Example 291B gave the title compound. ¹H NMR (500 MHz, DMSO-d₆) δ ppm 9.32 (d, J=2.5 Hz, 1H), 9.14 (s, 1H), 8.92 (s, 1H), 8.74 (s, 1H), 8.56-8.49 (m, 1H), 8.28 (s, 1H), 8.11 (d, J=8.6 Hz, 1H), 7.39 (d, J=2.8 Hz, 1H), 7.22 (dd, J=8.7, 2.7 Hz, 1H), 6.90 (d, J=8.7 Hz, 1H), 5.76-5.69 (m, 1H), 4.85-4.79 (m, 1H), 4.66-4.59 (m, 1H), 2.36 (s, 6H), 1.79-1.63 (m, 2H); MS (ESI⁻) m/z 546 (M−H)⁻.

### Example 294: Activity of Exemplary Compounds in an In Vitro Model of Vanishing Cell White Matter Disease (VWMD)

In order to test exemplary compounds of the invention in a cellular context, a stable VWMD cell line was first constructed. The ATF4 reporter was prepared by fusing the human full-length ATF4 5′-UTR (NCBI Accession No. BC022088.2) in front of the firefly luciferase (FLuc) coding sequence lacking the initiator methionine as described in Sidrauski et al (eLife 2013). The construct was used to produce recombinant retroviruses using standard methods and the resulting viral supernatant was used to transduce HEK293T cells, which were then subsequently selected with puromycin to generate a stable cell line.

HEK293T cells carrying the ATF4 luciferase reporter were plated on polylysine coated 384-well plates (Greiner Bio-one) at 30,000 cells per well. Cells were treated the next day with 1 μg/mL tunicamycin and 200 nM of a compound of Formula (I) for 7 hours. Luminescence was measured using One Glo (Promega) as specified by the manufacturer. Cells were maintained in DMEM with L-glutamine supplemented with 10% heat-inactivated FBS (Gibco) and Antibiotic-Antimycotic solution (Gibco).

Table 2 below summarizes the EC₅₀ data obtained using the ATF4-Luc assay for exemplary compounds of the invention. In this table, “A” represents an EC₅₀ of less than 10 nM; “B” an EC₅₀ of between 10 nM and 50 nM; “C” an EC₅₀ of between 50 nM and 250 nM; “D” an EC₅₀ of between 250 nM and 500 nM; “E” an EC₅₀ of between 500 nM and 2 μM; “F” an EC₅₀ of greater than 2 μM; and “G” indicates that data is not available.

TABLE 2
EC₅₀ values of exemplary compounds of
the invention in the ATF4-Luc assay.

| Compound No. | ATF4-Luc EC₅₀ |
|---|---|
| 100 | F |
| 101 | E |
| 102 | A |
| 103 | A |
| 104 | A |
| 105 | A |
| 106 | A |
| 107 | A |
| 108 | A |
| 109 | E |
| 110 | B |
| 111 | C |
| 112 | A |
| 113 | A |
| 114 | A |
| 115 | A |
| 116 | A |
| 117 | A |
| 118 | A |
| 119 | A |
| 120 | A |
| 121 | A |
| 122 | B |
| 123 | D |
| 124 | B |
| 125 | C |
| 126 | A |
| 127 | B |
| 128 | C |
| 129 | D |
| 130 | F |
| 131 | E |
| 132 | C |
| 133 | B |
| 134 | B |
| 135 | A |
| 136 | A |
| 137 | F |
| 138 | D |
| 139 | C |
| 140 | C |
| 141 | B |
| 142 | C |
| 143 | E |
| 144 | C |
| 145 | C |
| 146 | E |
| 147 | E |
| 148 | C |
| 149 | C |
| 150 | D |
| 151 | E |
| 152 | D |
| 153 | B |
| 154 | A |
| 155 | F |
| 156 | B |
| 157 | B |
| 158 | B |
| 159 | A |
| 160 | F |
| 161 | A |
| 162 | C |
| 163 | F |
| 164 | F |
| 165 | B |
| 166 | E |
| 167 | D |
| 168 | C |
| 169 | D |
| 170 | D |
| 171 | G |
| 172 | A |
| 173 | E |
| 174 | C |
| 175 | D |
| 176 | D |
| 177 | A |
| 178 | A |
| 179 | E |
| 180 | B |
| 181 | B |
| 182 | C |
| 183 | B |
| 184 | D |
| 185 | C |
| 186 | A |
| 187 | B |
| 188 | B |
| 189 | B |
| 190 | A |
| 191 | E |
| 192 | E |
| 193 | G |
| 194 | G |
| 195 | A |
| 196 | A |
| 197 | A |
| 198 | A |
| 199 | A |
| 199C | A |
| 200 | A |
| 201 | A |
| 202 | A |
| 203 | A |
| 204 | A |
| 205 | A |
| 206 | A |
| 207 | A |
| 207E | A |
| 208 | A |
| 209 | C |
| 210 | D |
| 211 | B |
| 212 | G |
| 213 | B |
| 214 | D |
| 215 | B |
| 216 | D |
| 217 | C |
| 218 | B |
| 219 | A |
| 220 | B |
| 221 | F |
| 222 | A |
| 223 | A |
| 224 | A |
| 225 | B |
| 226 | A |
| 227 | C |
| 228 | B |
| 229 | A |
| 230 | A |
| 231 | A |
| 232 | A |
| 233 | B |
| 234 | A |
| 235 | B |
| 236 | A |
| 237 | A |
| 238 | A |
| 239 | A |
| 240 | B |
| 241 | A |
| 242 | A |
| 243 | A |
| 244 | B |
| 245 | A |
| 246 | A |
| 247 | A |
| 248 | A |
| 249 | A |
| 250 | C |
| 251 | B |
| 252 | A |
| 253 | A |
| 254 | A |
| 255 | A |
| 256 | A |
| 257 | F |
| 258 | F |
| 259 | E |
| 260 | A |
| 261 | C |
| 262 | F |
| 263 | F |
| 264 | A |
| 265 | A |
| 266 | C |
| 267 | B |
| 268 | B |
| 269 | C |
| 270 | C |
| 271 | B |
| 272 | A |
| 273 | B |
| 274 | C |
| 275 | A |
| 276 | B |
| 277 | A |
| 278 | B |
| 279 | A |
| 280 | B |
| 281 | A |
| 282 | F |
| 283 | B |
| 284 | B |
| 285 | A |
| 286 | C |
| 287 | C |
| 288 | A |
| 289 | E |
| 290 | B |
| 291 | C |
| 292 | C |
| 293 | B |
| 294 | E |
| 295 | F |
| 296 | A |
| 297 | A |
| 298 | B |
| 299 | B |
| 300 | A |
| 301 | A |
| 302 | C |
| 303 | A |
| 304 | E |
| 305 | A |
| 306 | B |
| 307 | A |
| 308 | B |
| 309 | A |
| 310 | B |
| 311 | C |
| 312 | E |
| 313 | B |
| 314 | A |
| 315 | A |
| 316 | C |
| 317 | B |
| 318 | A |
| 319 | C |
| 320 | E |
| 321 | C |
| 322 | C |
| 323 | C |
| 324 | D |
| 325 | B |
| 326 | B |
| 327 | C |
| 328 | C |
| 329 | C |
| 330 | C |
| 331 | C |
| 332 | A |
| 333 | D |
| 334 | C |
| 335 | C |
| 336 | D |
| 337 | A |
| 338 | G |
| 339 | B |
| 340 | A |
| 341 | A |
| 342 | B |
| 343 | B |
| 344 | C |
| 345 | F |
| 346 | E |
| 347 | A |
| 348 | C |
| 349 | B |
| 350 | A |
| 351 | A |
| 352 | E |
| 353 | C |
| 354 | G |
| 355 | A |
| 356 | C |
| 357 | E |
| 358 | F |
| 359 | A |
| 360 | B |
| 361 | C |
| 362 | E |
| 363 | B |
| 364 | A |
| 365 | G |
| 366 | G |
| 367 | A |
| 368 | A |
| 369 | B |
| 370 | A |
| 371 | A |
| 372 | B |
| 373 | B |
| 374 | D |
| 375 | A |
| 376 | B |
| 377 | C |
| 378 | C |
| 379 | E |
| 380 | G |
| 381 | A |
| 382 | C |
| 383 | C |
| 384 | C |
| 385 | C |
| 386 | C |
| 387 | C |
| 388 | D |
| 389 | F |
| 390 | B |
| 391 | A |
| 392 | E |

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims are introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

We claim:

1. A compound of Formula (I): 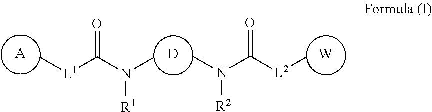
or a pharmaceutically acceptable salt, solvate, hydrate, tautomer, N-oxide, or stereoisomer thereof, wherein:
D is a bridged bicyclic cycloalkyl, bridged bicyclic heterocyclyl, or cubanyl, wherein each bridged bicyclic cycloalkyl, bridged bicyclic heterocyclyl, or cubanyl is optionally substituted on one or more available carbons with 1-4 R^{X}; and wherein if the bridged bicyclic heterocyclyl contains a substitutable nitrogen moiety, the substitutable nitrogen may be optionally substituted by R^{N1};
L¹ is a bond, C₁-C₆ alkylene, 2-7 membered heteroalkylene, —NR^{N2}—, or —O—, wherein C₁-C₆ alkylene or 2-7 membered heteroalkylene is optionally substituted with 1-5 R^{L1};
L² is a bond, C₁-C₆ alkylene, or 2-7 membered heteroalkylene, wherein C₁-C₆ alkylene or 2-7 membered heteroalkylene is optionally substituted with 1-5 R^{L2};
R¹ is hydrogen or C₁-C₆ alkyl;
R² is hydrogen or C₁-C₆ alkyl;
W is a 8-10 membered, partially unsaturated, fused bicyclic ring moiety comprising a 5-6 membered heterocyclyl fused to a phenyl or 5-6-membered heteroaryl; wherein the heterocyclyl may be optionally substituted on one or more available carbons with 1-4 R^{W1}; and wherein the phenyl or heteroaryl may optionally be substituted on one or more available unsaturated carbons with 1-4 R^{W2}; and wherein if the heterocyclyl contains a substitutable nitrogen moiety, the substitutable nitrogen may optionally be substituted with R^{N3};
A is phenyl or 5-6-membered heteroaryl, wherein phenyl or 5-6-membered heteroaryl is optionally substituted on one or more available carbons with 1-5 R^{Y}; and wherein if the 5-6-membered heteroaryl contains a substitutable nitrogen moiety, the substitutable nitrogen may be optionally substituted by R^{N4};
each R^{L1} is independently selected from the group consisting of hydrogen, C₁-C₆ alkyl, hydroxy-C₁-C₆ alkyl, halo-C₁-C₆ alkyl, amino-C₁-C₆ alkyl, cyano-C₁-C₆ alkyl, oxo, halo, cyano, —OR^{A}, —NR^{B}R^{C}, —NR^{B}C(O)R^{D}, —C(O)NR^{B}R^{C}, —C(O)R^{D}, —C(O)OH, —C(O)OR^{D}, —SR^{E}, —S(O)R^{D}, and —S(O)₂R^{D};
each R^{L2} is independently selected from the group consisting of hydrogen, C₁-C₆ alkyl, hydroxy-C₁-C₆ alkyl, halo-C₁-C₆ alkyl, amino-C₁-C₆ alkyl, cyano-C₁-C₆ alkyl, oxo, halo, cyano, —OR^{A}, —NR^{B}R^{C}, —NR^{B}C(O)R^{D}, —C(O)NR^{B}R^{C}, —C(O)R^{D}, —C(O)OH, —C(O)OR^{D}, —SR^{E}, —S(O)R^{D}, and —S(O)₂R^{D};
R^{N1} is selected from the group consisting of hydrogen, C₁-C₆ alkyl, hydroxy-C₂-C₆ alkyl, halo-C₂-C₆ alkyl, amino-C₂-C₆ alkyl, cyano-C₂-C₆ alkyl, —C(O)NR^{B}R^{C}, —C(O)R^{D}, —C(O)OR^{D}, and —S(O)₂R^{D};
R^{N2} is selected from the group consisting of hydrogen, C₁-C₆ alkyl, hydroxy-C₂-C₆ alkyl, halo-C₂-C₆ alkyl, amino-C₂-C₆ alkyl, cyano-C₂-C₆ alkyl, —C(O)NR^{B}R^{C}, —C(O)R^{D}, —C(O)OR^{D}, and —S(O)₂R^{D};
R^{N3} is selected from the group consisting of hydrogen, C₁-C₆ alkyl, hydroxy-C₂-C₆ alkyl, C₁-C₆ alkyl-C₁-C₆ cycloalkyl, C₁-C₆ alkenyl, —C(O)—C₁-C₆ alkyl, —C(O)—C₁-C₆ cycloalkyl, C₁-C₆ alkyl-CO₂H, C₁-C₆ alkyl-CO₂—C₁-C₆ alkyl, —C(O)—C₁-C₃ alkyl-O—C₁-C₃ alkyl-O—C₁-C₃ alkyl, —C(O)-phenyl, —C(O)-heteroaryl, —C(O)-heterocyclyl, —S—C₁-C₆ alkyl, —S(O)₂—C₁-C₆ alkyl, —S(O)₂-phenyl, —S(O)₂-heteroaryl, —C(O)NR^{B}R^{C} and —C(O)OR^{D};
wherein C₁-C₆ alkyl, hydroxy-C₂-C₆ alkyl, C₁-C₆ alkyl-C₁-C₆ cycloalkyl, C₁-C₆ alkenyl, C(O)—C₁-C₆ alkyl, —C(O)—C₁-C₆ cycloalkyl, C₁-C₆ alkyl-CO₂H, C₁-C₆ alkyl-CO₂—C₁-C₆ alkyl, —C(O)-heterocyclyl, —S—C₁-C₆ alkyl and —S(O)₂—C₁-C₆ alkyl may optionally be substituted by one or more substituents each independently selected from the group consisting of fluoro, hydroxyl, C₁-C₆ alkoxy, C₁-C₆ alkyl (optionally substituted by one, two or three fluorine atoms) and S(O)_{w}C₁₋₆ alkyl (wherein w is 0, 1 or 2); and
wherein —C(O)-phenyl, —C(O)-heteroaryl, —S(O)₂-phenyl and —S(O)₂-heteroaryl may optionally be substituted by one or more substituents each independently selected from the group consisting of halogen, hydroxyl, C₁-C₆ alkyl (optionally substituted by one, two or three fluorine atoms), C₁-C₆ alkoxy (optionally substituted by one, two or three fluorine atoms), S(O₂)NR^{B}R^{C} and SO₂F;
R^{N4} is selected from the group consisting of hydrogen, C₁-C₆ alkyl, hydroxy-C₂-C₆ alkyl, halo-C₂-C₆ alkyl, amino-C₂-C₆ alkyl, cyano-C₂-C₆ alkyl, C₃-C₆ cycloalkyl, phenyl, 5-6-membered heteroaryl, —C(O)NR^{B}R^{C}, —C(O)R^{D}, —C(O)OR^{D}, and —S(O)₂R^{D};
wherein C₃-C₆ cycloalkyl, phenyl, and 5-6-membered heteroaryl may optionally be substituted by one or more substituents each independently selected from the group consisting of halo, C₁-C₆ alkyl (optionally substituted by one, two or three fluorine atoms), and C₁-C₆ alkoxy (optionally substituted by one, two or three fluorine atoms).
each R^{W1} is independently selected from the group consisting of hydrogen, C₁-C₆ alkyl (optionally substituted by —CO₂H), hydroxy-C₁-C₆ alkyl, hydroxy-C₂-C₆ alkyl-O—, halo-C₁-C₆ alkyl, amino-C₁-C₆ alkyl, cyano-C₁-C₆ alkyl, oxo, C═NOH, halo, cyano, —OR^{A}, —NR^{B}R^{C}, —NR^{B}R^{CC}, —R^{B}C(O)R^{D}, —C(O)NR^{B}R^{C}, —C(O)R^{D}, —C(O)OH, —C(O)OR^{D}, —SR^{E}, —S(O)R^{D}, and —S(O)₂R^{D};
each R^{W2} is independently selected from the group consisting of hydrogen, C₁-C₆ alkyl, hydroxy-C₁-C₆ alkyl, hydroxy-C₂-C₆ alkyl-O—, halo-C₁-C₆ alkyl, halo-C₁-C₆ alkoxy, amino-C₁-C₆ alkyl, cyano-C₁-C₆ alkyl, halo, cyano, —OR^{A}, —NR^{B}R^{C}, —N^{B}C(O)R^{D}, —C(O)NR^{B}R^{C}, —C(O)R^{D}, —C(O)OH, —C(O)OR^{D}, —S(R^{F})ₘ, —S(O)R^{D}, and —S(O)₂R^{D}; or
2 R^{W2} groups on adjacent atoms, together with the atoms to which they are attached, form a 3-7-membered fused cycloalkyl, 3-7-membered fused heterocyclyl, fused aryl, or 5-6 membered fused heteroaryl, each of which is optionally substituted with 1-5 R^{X};
each R^{X} is independently selected from the group consisting of hydrogen, C₁-C₆ alkyl, hydroxy-C₁-C₆ alkyl, halo-C₁-C₆ alkyl, amino-C₁-C₆ alkyl, cyano-C₁-C₆ alkyl, oxo, halo, cyano, —OR^{A}, —NR^{B}R^{C}, —N^{B}C(O)R^{D}, —C(O)NR^{B}R^{C}, —C(O)R^{D}, —C(O)OH, —C(O)OR^{D}, —SR^{E}, —S(O)R^{D}, and —S(O)₂R^{D};
each R^{Y} is independently selected from the group consisting of hydrogen, C₁-C₆ alkyl, hydroxy-C₁-C₆ alkyl, halo-C₁-C₆ alkyl, halo-C₁-C₆ alkoxy, amino-C₁-C₆ alkyl, cyano-C₁-C₆ alkyl, C₃-C₆ cycloalkyl, 3-7 membered heterocyclyl, halo-C₁-C₆ alkyl-3-7 membered heterocyclyl, halo, cyano, —OR^{A}, —NR^{B}R^{C}, —N^{B}C(O)R^{D}, —C(O)NR^{B}R^{C}, —C(O)R^{D}, —C(O)OH, —C(O)OR^{D}, —S(R^{F})ₘ, —S(O)R^{D}, —S(O)₂R^{D}, and G¹; or
2 R^{Y} groups on adjacent atoms, together with the atoms to which they are attached form a 3-7-membered fused cycloalkyl, 3-7-membered fused heterocyclyl, fused aryl, or 5-6 membered fused heteroaryl, each of which is optionally substituted with 1-5 R^{X};
each G¹ is independently 3-7-membered cycloalkyl, 3-7-membered heterocyclyl, aryl, or 5-6-membered heteroaryl, wherein each 3-7-membered cycloalkyl, 3-7-membered heterocyclyl, aryl, or 5-6-membered heteroaryl is optionally substituted with 1-3 R^{Z};
each R^{Z} is independently selected from the group consisting of C₁-C₆ alkyl, hydroxy-C₁-C₆ alkyl, halo-C₁-C₆ alkyl, halo, cyano, —OR^{A}, —NR^{B}R^{C}, —NR^{B}C(O)R^{D}, —C(O)NR^{B}R^{C}, —C(O)R^{D}, —C(O)OH, —C(O)OR^{D}, and —S(O)₂R^{D};
R^{A} is, at each occurrence, independently hydrogen, C₁-C₆ alkyl, halo-C₁-C₆ alkyl, —C(O)NR^{B}R^{C}, —C(O)R^{D}, or —C(O)OR^{D};
each of R^{B} and R^{C} is independently hydrogen or C₁-C₆ alkyl;
R^{B} and R^{C} together with the atom to which they are attached form a 3-7-membered heterocyclyl ring optionally substituted with 1-3 R^{Z};
each R^{CC} is independently selected from the group consisting of hydroxy-C₁-C₆ alkyl, halo-C₁-C₆ alkyl, C₁-C₆ alkyl-CO₂H, C₁-C₆ alkyl-CO₂—C₁-C₆ alkyl, C(O) C₁-C₆ alkyl, S(O)₂—C₁-C₆ alkyl, 3-6-membered cycloalkyl and 4-6-membered heterocyclyl; wherein 3-6-membered cycloalkyl and 4-6-membered heterocyclyl may optionally be substituted by one or more substituents each independently selected from the group consisting of C₁-C₆ alkyl, hydroxy-C₁-C₆ alkyl, halo-C₁-C₆ alkyl, hydroxyl, halo and —C(O)OH;
each R^{D} is independently C₁-C₆ alkyl or halo-C₁-C₆ alkyl;
each R^{E} is independently hydrogen, C₁-C₆ alkyl, or halo-C₁-C₆ alkyl;
each R^{F} is independently hydrogen, C₁-C₆ alkyl, or halo; and
m is 1 when R^{F} is hydrogen or C₁-C₆ alkyl, or 5 when R^{F} is halo.

2. The compound of claim 1, wherein D is a bridged bicyclic cycloalkyl or bridged bicyclic heterocyclyl, wherein each bridged bicyclic cycloalkyl or bridged bicyclic heterocyclyl may optionally be substituted by 1-4 R^{X}.

3. The compound of any one of claims 1-2, wherein D is a bridged bicyclic 5-8 membered cycloalkyl or a bridged bicyclic 5-8-membered heterocyclyl, wherein each bridged bicyclic 5-8-membered cycloalkyl or bridged bicyclic 5-8-membered heterocyclyl may optionally be substituted by 1-4 R^{X}.

4. The compound of any one of claims 1-3, wherein D is bicyclo[1.1.1]pentane, bicyclo[2.2.1]heptane, bicyclo[2.1.1]hexane, bicyclo[2.2.2]octane, bicyclo[3.2.1]octane, 7-oxabicyclo[2.2.1]heptane, 2-oxabicyclo[2.2.2]octane, or 2-azabicyclo[2.2.2]octane, each of which may optionally be substituted by 1-4 R^{X} groups.

5. The compound of any one of claims 1-4, wherein D is

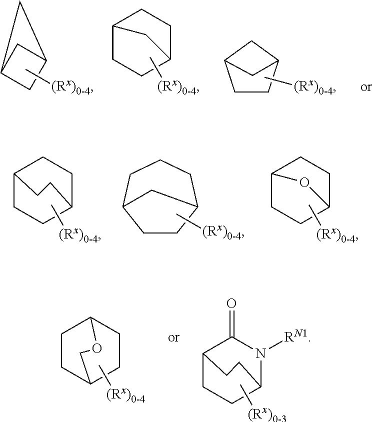

6. The compound of any one of claims 1-5, wherein D is

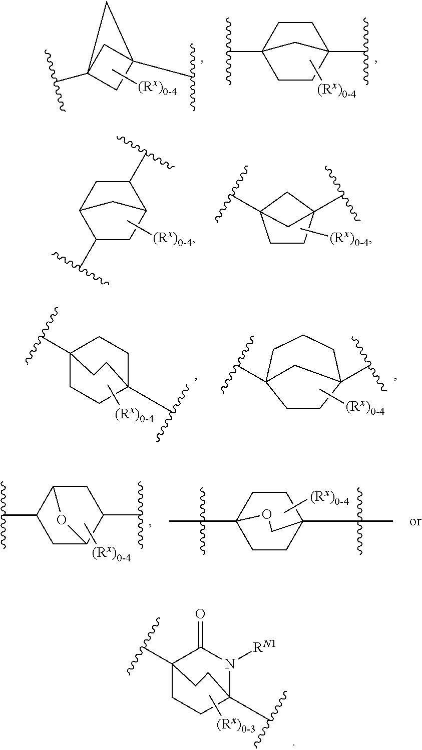

7. The compound of any one of claims 1-5, wherein D is

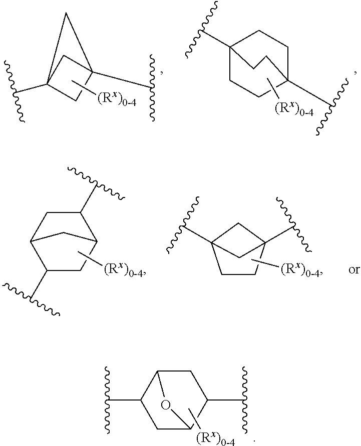

8. The compound of any one of claims 1-7, wherein D is substituted with 0 R^{X}.

9. The compound of any one of claims 1-8, wherein D is

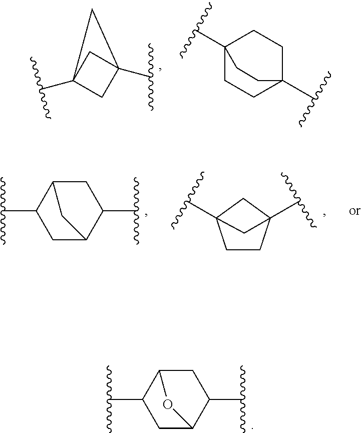 [SMILES: *C12CC(*)(C1)C2 ; *C12CCC(*)(CC1)CC2 ; *C1CC2CC1CC2* ; *C12CCC(*)(C1)C2 ; *C1CC2OC1CC2*]

10. The compound of any one of claims 1-9, wherein D is

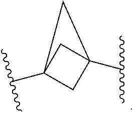 [SMILES: *C12CC(*)(C1)C2]

11. The compound of any one of claims 1-9, wherein D is

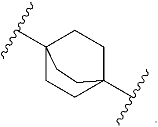 [SMILES: *C12CCC(*)(CC1)CC2]

12. The compound of any one of claims 1-7, wherein D is substituted with 1 or 2 R^{X}.

13. The compound of any one of claims 1-7 and 12, wherein D is

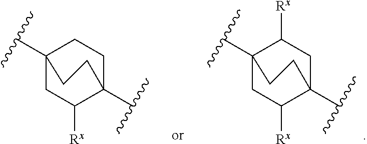

14. The compound of any one of claims 12-13, wherein each R^{X} is independently selected from the group consisting of oxo, —OH, —C(O)OH, —C(O)OR^{D}, halo, and hydroxy-C₁-C₆ alkyl.

15. The compound of any one of claims 1-14, wherein L¹ is a bond, 2-7 membered heteroalkylene, —NR^{N2}—, or —O—, wherein 2-7 membered heteroalkylene is optionally substituted by 1-5 R^{L1}.

16. The compound of any one of claims 1-15, wherein L¹ is a bond, 2-7 membered heteroalkylene, —NR^{N2}—, or —O—, wherein 2-7 membered heteroalkylene is substituted by 0 R^{L}.

17. The compound of any one of claims 1-16, wherein L¹ is selected from a bond and CH₂O—*, wherein “-*” indicates the attachment point to A.

18. The compound of any one of claims 1-17, wherein R¹ is hydrogen or CH₃.

19. The compound of any one of claims 1-18, wherein R² is hydrogen or CH₃.

20. The compound of any one of claims 1-19, wherein A is selected from the group consisting of phenyl, pyrazinyl, isoxazolyl, pyrimidinyl, oxazolyl, thiazolyl and pyridyl, each of which is optionally substituted with 1-2 R^{Y} groups; or
A is pyrazolyl optionally substituted by R^{N4}.

21. The compound of any one of claims 1-20, wherein A is selected from the group consisting of:

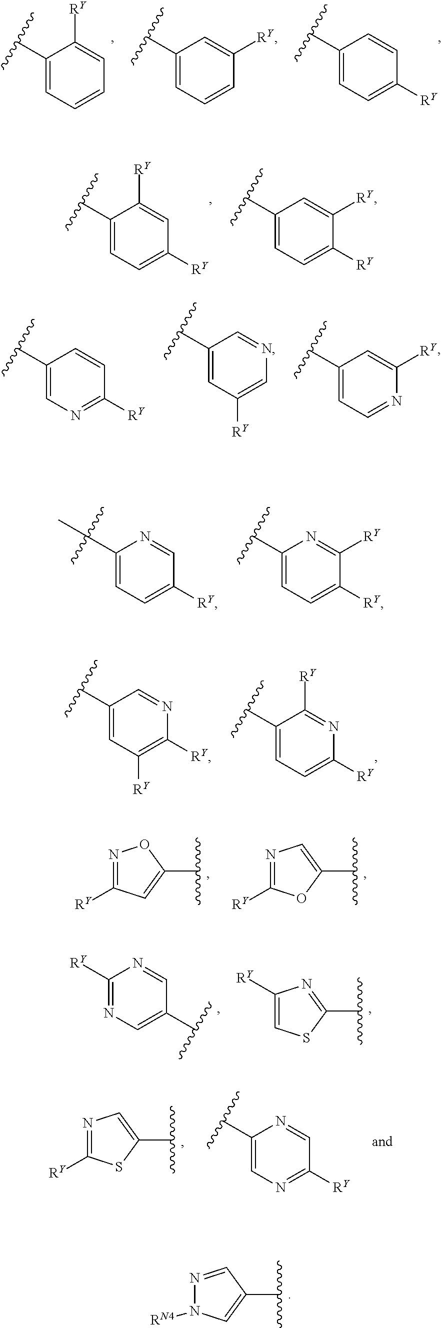

22. The compound of any one of claims 1-21, wherein each R^{Y} is independently selected from the group consisting of hydrogen, chloro, fluoro, CHF₂, CF₃, CH₃, CH₂CH₃, CH(CH₃)₂, 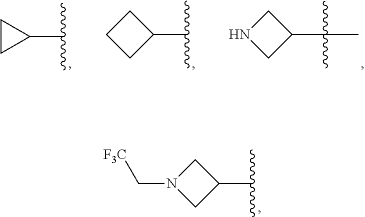
OCH₃, OCHF₂, OCF₃, OCH₂CF₃, OCH(CH₃)₂, and CN.

23. The compound of any one of claims 1-21, wherein 2 R^{Y} on adjacent carbons, together with the atoms to which they are attached form a 1,3-dioxolanyl ring, which is optionally substituted with 1-2 R^{X}.

24. The compound of claim 23, wherein each R^{X} is independently fluoro.

25. The compound of any one of claims 1-21, wherein R^{N4} is selected from the group consisting of hydrogen, phenyl (optionally substituted by one or more halo atoms), pyridyl (optionally substituted by CF₃), and cyclobutyl (optionally substituted by OCF₃).

26. The compound of any one of claims 1-25, wherein L² is a bond or C₁-C₆ alkylene, wherein C₁-C₆ alkylene is optionally substituted by 1-5 R^{L2}.

27. The compound of any one of claims 1-26, wherein L² is a bond or C₁-C₆ alkylene, wherein C₁-C₆ alkylene is optionally substituted by 0 R^{L2}.

28. The compound of any one of claims 1-27, wherein L² is selected from a bond or —CH₂—.

29. The compound of any one of claims 1-28, wherein L² is a bond.

30. The compound of any one of claims 1-29, wherein W is represented by Formula (W-a): 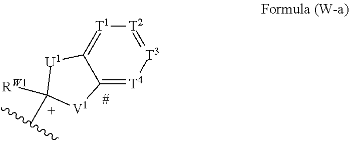
wherein:
T¹ is nitrogen or C(R^{W2});
T² is nitrogen or C(R^{W2});
T³ is nitrogen or C(R^{W2});
T⁴ is nitrogen or C(R^{W2});
wherein no more than two of T¹, T², T³, and T⁴ may be nitrogen;
U¹ is selected from the group consisting of a bond, —O—, —CO—, —NR^{N3}—, and —S(O)_{w}— (wherein w is 0, 1, or 2);
V¹ is selected from the group consisting of ⁺—O—^{#}, ⁺—C(R^{V11}R^{V12})—^{#}, ⁺—C(R^{V11}R^{V12})—C(O)—^{#}, ⁺—C(R^{V11}R^{V12})—C(═N—OH)—^{#}, ⁺—C(R^{V11}R^{V12})—C(R^{V13}R^{V14})—^{#}, ⁺—C(R^{V15}R^{V16})—O—^{#}, ⁺—C(R^{V15}R^{V16})—NR^{N3}—^{#}, ⁺—C(O)—NR^{N3}—^{#}, ⁺—NR^{N3}—^{#}, ⁺—O—C(R^{V15}R^{V16})—^{#}, ⁺—NR^{N3}C(R^{V15}R^{V16})—^{#}, ⁺—NR^{N3}C(O)—^{#}, ⁺—C(O)—O—^{#}, ⁺—O—C(O)—^{#}, ⁺—C(R^{V15}R^{V16})—S(O)_{w}—^{#}, ⁺S(O)_{w}—C(R^{V15}R^{V16})—^{#} (wherein w is 0, 1, or 2) and 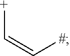
wherein the “⁺-” and “-^{#}” indicate the attachment points of V¹ as indicated in Formula (W-a);
wherein if V¹ is ⁺—O—^{#}, ⁺—NR^{N3}—^{#} or ⁺—C(R^{V11}R^{V12})—^{#}; U¹ is not a bond;
R^{V11} and R^{V12} are each independently selected from the group consisting of hydrogen, C₁-C₆ alkyl, hydroxy-C₁-C₆ alkyl, halo-C₁-C₆ alkyl, amino-C₁-C₆ alkyl, cyano-C₁-C₆ alkyl, halo, cyano, —OR^{A}, —NR^{B}R^{C}, —NR^{B}R^{CC}, —NR^{B}C(O)R^{D}, —C(O)NR^{B}R^{C}, —C(O)R^{D}, —C(O)OH, —C(O)OR^{D}, —SR^{E}, —S(O)R^{D}, and —S(O)₂R^{D};
R^{V13} and R^{V14} are each independently selected from the group consisting of hydrogen, C₁-C₆ alkyl, hydroxy-C₁-C₆ alkyl, halo-C₁-C₆ alkyl, amino-C₁-C₆ alkyl, cyano-C₁-C₆ alkyl, halo, cyano, —OR^{A}, —NR^{B}R^{C}, —NR^{B}R^{CC}, —NR^{B}C(O)R^{D}, —C(O)NR^{B}R^{C}, —C(O)R^{D}, —C(O)OH, —C(O)OR^{D}, —SR^{E}, —S(O)R^{D}, and —S(O)₂R^{D};
R^{V15} and R^{V16} are each independently selected from the group consisting of hydrogen, C₁-C₆ alkyl, hydroxy-C₂-C₆ alkyl, halo-C₂-C₆ alkyl, amino-C₂-C₆ alkyl, cyano-C₂-C₆ alkyl, —C(O)NR^{B}R^{C}, —C(O)R^{D}, —C(O)OH, and —C(O)OR^{D}; and
R^{W1} is selected from the group consisting of hydrogen and C₁-C₆ alkyl.

31. The compound of claim 30, wherein W is represented by Formula (W-a-1), Formula (W-a-2), Formula (W-a-3), Formula (W-a-4), or Formula (W-a-5):

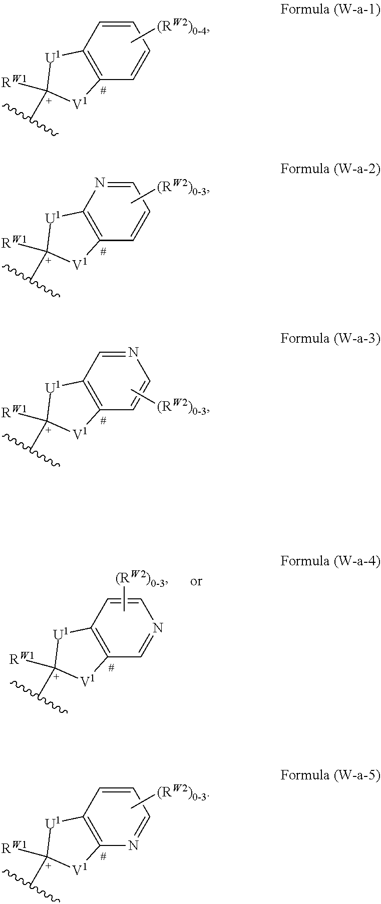

32. The compound of claim 31, wherein W is represented by Formula (W-a-1):

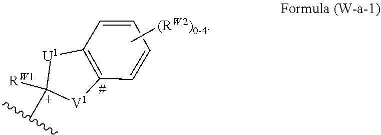

33. The compound of any one of claims 30-32, wherein U¹ is selected from the group consisting of a bond, —O—, —CO— and —NR^{N3}—; and V¹ is selected from the group consisting of ⁺—O—^{#}, ⁺—C(R^{V11}R^{V12})—^{#}, ⁺—C(R^{V11}R^{V12})—C(R^{V13}R^{V14})—^{#}, ⁺—C(R^{V15}R^{V16})—O—^{#}, ⁺—C(R^{V11}R^{V12})—C(O)—^{#}, ⁺—C(R^{V11}R^{V12})—C(═N—OH)—^{#}, ⁺—O—C(R^{V15}R^{V16})—^{#}, ⁺—C(R^{V15}R^{V16})—NR^{N3}—^{#}, ⁺—C(O)—NR^{N3}—^{#} and 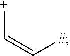
wherein “⁺-” and “-^{#}” indicate the attachment points of V¹ as indicated in Formula (W-a); and wherein if V¹ is ⁺—O—^{#} or ⁺—C(R^{V11}R^{V12})—^{#}, U¹ is not a bond.

34. The compound of any one of claims 30-33, wherein each of R^{V11}, R^{V12}, R^{V13}, and R^{V14} is independently selected from the group consisting of hydrogen, halo, C₁-C₃ alkyl, cyano, —OR^{A}, —NR^{B}R^{C} and —NR^{B}R^{CC}.

35. The compound of any one of claims 30-34, wherein each of R^{V11}, R^{V12}, R^{V13}, and R^{V14} is independently selected from the group consisting of hydrogen, hydroxyl, C₁-C₃ alkyl, —O—C₁-C₃ alkyl, —NR^{B}R^{C} and —NR^{B}R^{CC}.

36. The compound of any one of claims 30-35, wherein each of R^{V15} and ^{V16} is independently selected from the group consisting of hydrogen and C₁-C₃ alkyl.

37. The compound of any one of claims 30-36, wherein each of R^{V15} and R^{V16} is hydrogen.

38. The compound of any one of claims 30-37, wherein R^{V13} is selected from the group consisting of hydrogen, hydroxyl, CH₃, OCH₃, NH(CH₂)₂₀H, NH(CH₂)₂CO₂H, NH(CH₂)₂CO₂CH₃, NH—SO₂—CH₃, NH(CO)CH₃, NH₂, NHCH₃, NHCH₂CF₃,

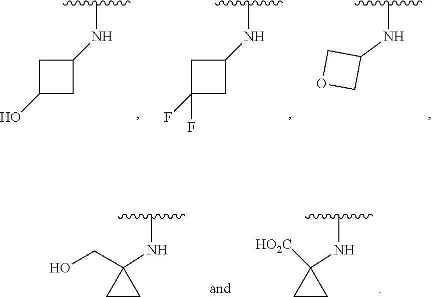 [SMILES: *NC1CC(O)C1 ; *NC1CC(F)(F)C1 ; *NC1COC1 ; *NC1(CO)CC1 ; *NC1(C(=O)O)CC1]

39. The compound of any one of claims 30-38, wherein R^{V12} is selected from the group consisting of hydrogen and C₁-C₃ alkyl.

40. The compound of any one of claims 30-39, wherein W is a benzo[d][1,3]dioxole, 3,4-dihydro-2H-benzo[b][1,4]oxazine, chromane, chromene, chroman-4-one, chroman-4-ol, chroman-4-one oxime, 2H-benzo[b][1,4]oxazin-3(4H)-one, 2,3-dihydrobenzo[b][1,4]dioxine, indoline, 2,3-dihydrobenzofuran or benzofuran-3(2H)-one moiety; wherein each of which is attached to L² through a saturated carbon atom, and wherein each of which is optionally substituted on one or more available unsaturated carbons with 1-4 R^{W2}, and wherein each R^{W2} is independently selected from the group consisting of C₁-C₆ alkyl, halo-C₁-C₆ alkyl, halo, oxo, cyano, and —OR^{A}.

41. The compound of any one of claims 30-40, wherein W is selected from the group consisting of:

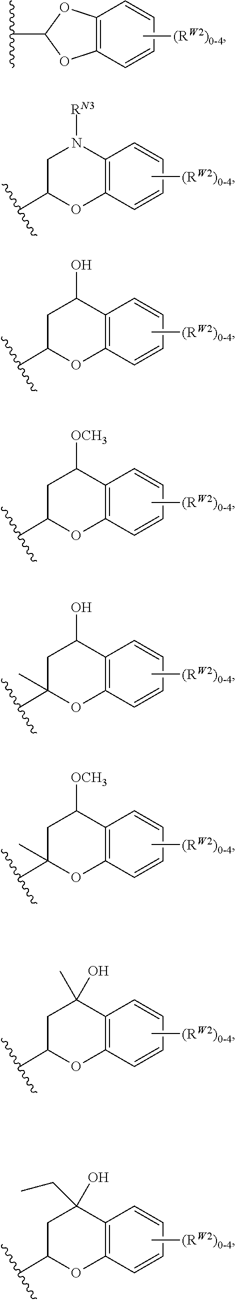

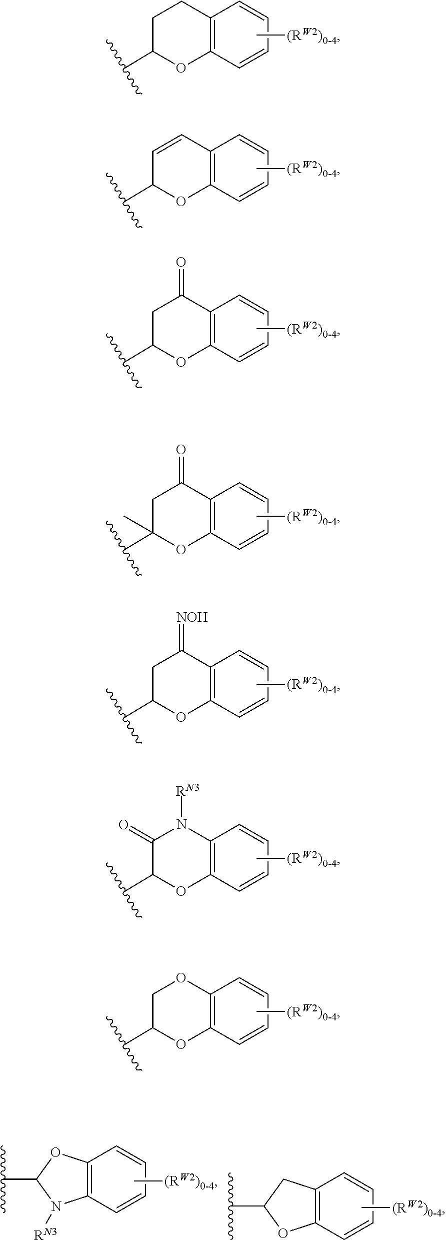

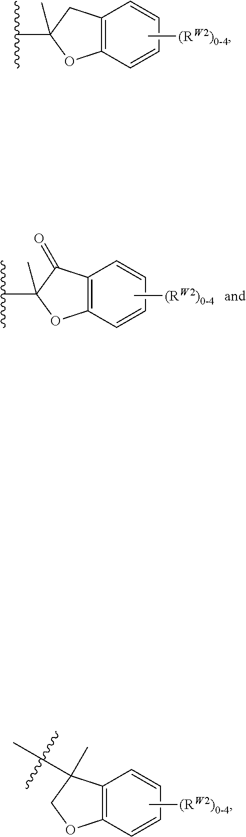

42. The compound of any one of claims 1-29, wherein W is represented by Formula (W-b): 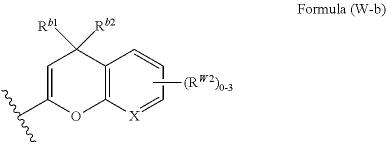
wherein:
X is nitrogen or C(R^{W2});
R^{b1} is hydrogen;
R^{b2} is hydroxyl; or
R^{b1} and R^{b2} taken together form an oxo moiety;
each R^{W2} is independently selected from the group consisting of hydrogen, C₁-C₆ alkyl, hydroxy-C₁-C₆ alkyl, hydroxy-C₂-C₆ alkyl-O—, halo-C₁-C₆ alkyl, halo-C₁-C₆ alkoxy, amino-C₁-C₆ alkyl, cyano-C₁-C₆ alkyl, halo, cyano, —OR^{A}, —NR^{B}R^{C}, —N^{B}C(O)R^{D}, —C(O)NR^{B}R^{C}, —C(O)R^{D}, —C(O)OH, —C(O)OR^{D}, —S(R^{F})ₘ, —S(O)R^{D}, and —S(O)₂R^{D}; or
2 R^{W2} groups on adjacent atoms, together with the atoms to which they are attached, form a 3-7-membered fused cycloalkyl, 3-7-membered fused heterocyclyl, fused aryl, or 5-6 membered fused heteroaryl, each of which is optionally substituted with 1-5 R^{X};
each R^{X} is independently selected from the group consisting of hydrogen, C₁-C₆ alkyl, hydroxy-C₁-C₆ alkyl, halo-C₁-C₆ alkyl, amino-C₁-C₆ alkyl, cyano-C₁-C₆ alkyl, oxo, halo, cyano, —OR^{A}, —NR^{B}R^{C}, —N^{B}C(O)R^{D}, —C(O)NR^{B}R^{C}, —C(O)R^{D}, —C(O)OH, —C(O)OR^{D}, —SR^{E}, —S(O)R^{D}, and —S(O)₂R^{D};
R^{A} is, at each occurrence, independently hydrogen, C₁-C₆ alkyl, halo-C₁-C₆ alkyl, —C(O)NR^{B}R^{C}, —C(O)R^{D}, or —C(O)OR^{D};
each of R^{B} and R^{C} is independently hydrogen or C₁-C₆ alkyl;
R^{B} and R^{C} together with the atom to which they are attached form a 3-7-membered heterocyclyl ring optionally substituted with 1-3 R^{Z};
each R^{CC} is independently selected from the group consisting of hydroxy-C₁-C₆ alkyl, halo-C₁-C₆ alkyl, C₁-C₆ alkyl-CO₂H, C₁-C₆ alkyl-CO₂—C₁-C₆ alkyl, C(O) C₁-C₆ alkyl, S(O)₂—C₁-C₆ alkyl and 3-6-membered cycloalkyl; wherein 3-6-membered cycloalkyl may optionally be substituted by one or more substituents each independently selected from the group consisting of hydroxyl, halogen and —C(O)OH;
each R^{D} is independently C₁-C₆ alkyl or halo-C₁-C₆ alkyl;
each R^{E} is independently hydrogen, C₁-C₆ alkyl, or halo-C₁-C₆ alkyl;
each R^{F} is independently hydrogen, C₁-C₆ alkyl, or halo;
each R^{Z} is independently selected from the group consisting of C₁-C₆ alkyl, hydroxy-C₁-C₆ alkyl, halo-C₁-C₆ alkyl, halo, cyano, —OR^{A}, —NR^{B}R^{C}, —N^{B}C(O)R^{D}, —C(O)NR^{B}R^{C}, —C(O)R^{D}, —C(O)OH, —C(O)OR^{D}, and —S(O)₂R^{D}; and
m is 1 when RE is hydrogen or C₁-C₆ alkyl, 3 when RE is C₁-C₆ alkyl, or 5 when R^{F} is halo.

43. The compound of claim 42, wherein X is C(R^{W2}).

44. The compound of claim 42 or 43, wherein R^{b1} and R^{b2} taken together form an oxo moiety.

45. The compound of any one of claims 42-44, wherein the compound is represented by:

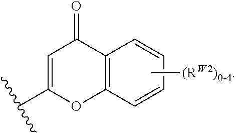

46. The compound of claim 42 or 43, wherein R^{b1} is hydrogen and R^{b2} is hydroxyl.

47. The compound of claim 46, wherein the compound is represented by:

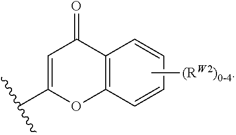

48. The compound of any one of claims 42-47, wherein each R^{W2} is independently selected from the group consisting of C₁-C₆ alkyl, halo-C₁-C₆ alkyl, halo, cyano, and —OR^{A}, or
2 R^{W2} groups on adjacent carbons, together with the atoms to which they are attached form a 1,3-dioxolanyl ring, which is optionally substituted with 1-2 fluorine atoms.

49. The compound of any one of claims 1-29, wherein W is represented by Formula (W-c): 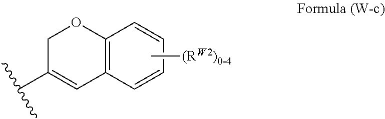
wherein:
each R^{W2} is independently selected from the group consisting of hydrogen, C₁-C₆ alkyl, hydroxy-C₁-C₆ alkyl, hydroxy-C₂-C₆ alkyl-O—, halo-C₁-C₆ alkyl, halo-C₁-C₆ alkoxy, amino-C₁-C₆ alkyl, cyano-C₁-C₆ alkyl, halo, cyano, —OR^{A}, —NR^{B}R^{C}, —NR^{B}C(O)R^{D}, —C(O)NR^{B}R^{C}, —C(O)R^{D}, —C(O)OH, —C(O)OR^{D}, —S(R^{F})ₘ, —S(O)R^{D}, and —S(O)₂R^{D}; or
2 R^{W2} groups on adjacent atoms, together with the atoms to which they are attached, form a 3-7-membered fused cycloalkyl, 3-7-membered fused heterocyclyl, fused aryl, or 5-6 membered fused heteroaryl, each of which is optionally substituted with 1-5 R^{X};
each R^{X} is independently selected from the group consisting of hydrogen, C₁-C₆ alkyl, hydroxy-C₁-C₆ alkyl, halo-C₁-C₆ alkyl, amino-C₁-C₆ alkyl, cyano-C₁-C₆ alkyl, oxo, halo, cyano, —OR^{A}, —NR^{B}R^{C}, —NR^{B}C(O)R^{D}, —C(O)NR^{B}R^{C}, —C(O)R^{D}, —C(O)OH, —C(O)OR^{D}, —SR^{E}, —S(O)R^{D}, and —S(O)₂R^{D};
R^{A} is, at each occurrence, independently hydrogen, C₁-C₆ alkyl, halo-C₁-C₆ alkyl, —C(O)NR^{B}R^{C}, —C(O)R^{D}, or —C(O)OR^{D};
each of R^{B} and R^{C} is independently hydrogen or C₁-C₆ alkyl;
R^{B} and R^{C} together with the atom to which they are attached form a 3-7-membered heterocyclyl ring optionally substituted with 1-3 R^{Z};
each R^{CC} is independently selected from the group consisting of hydroxy-C₁-C₆ alkyl, halo-C₁-C₆ alkyl, C₁-C₆ alkyl-CO₂H, C₁-C₆ alkyl-CO₂—C₁-C₆ alkyl, C(O) C₁-C₆ alkyl, S(O)₂—C₁-C₆ alkyl and 3-6-membered cycloalkyl; wherein 3-6-membered cycloalkyl may optionally be substituted by one or more substituents each independently selected from the group consisting of hydroxyl, halogen and —C(O)OH;
each R^{D} is independently C₁-C₆ alkyl or halo-C₁-C₆ alkyl;
each R^{E} is independently hydrogen, C₁-C₆ alkyl, or halo-C₁-C₆ alkyl;
each R^{F} is independently hydrogen, C₁-C₆ alkyl, or halo;
each R^{Z} is independently selected from the group consisting of C₁-C₆ alkyl, hydroxy-C₁-C₆ alkyl, halo-C₁-C₆ alkyl, halo, cyano, —OR^{A}, —NR^{B}R^{C}, —N^{B}C(O)R^{D}, —C(O)NR^{B}R^{C}, —C(O)R^{D}, —C(O)OH, —C(O)OR^{D}, and —S(O)₂R^{D}; and
m is 1 when R^{F} is hydrogen or C₁-C₆ alkyl, 3 when R^{F} is C₁-C₆ alkyl, or 5 when R^{F} is halo.

50. The compound of claim 49, wherein each R^{W2} is independently selected from the group consisting of C₁-C₆ alkyl, halo-C₁-C₆ alkyl, halo, cyano, and —OR^{A}, or
2 R^{W2} groups on adjacent carbons, together with the atoms to which they are attached form a 1,3-dioxolanyl ring, which is optionally substituted with 1-2 fluorine atoms.

51. The compound of any one of claims 1-29, wherein W is represented by Formula (W-d): 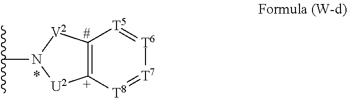
wherein:
T⁵ is nitrogen or C(R^{W2});
T⁶ is nitrogen or C(R^{W2});
T⁷ is nitrogen or C(R^{W2});
T⁸ is nitrogen or C(R^{W2});
wherein no more than two of T⁵, T⁶, T⁷, and T⁸ may be nitrogen;
V² is selected from the group consisting of ^{*}—C(R^{V21}R^{V22})—^{#}, ^{*}—C(R^{V21}R^{V22})—C(R^{V23}R^{V24})—^{#}, ^{*}—C(R^{V21}R^{V22})—C(R^{V23}R^{V24})—C(R^{V23}R^{V24})—^{#}, ^{*}—C(R^{V21}R^{V22})—C(R^{V21}R^{V22})—O—^{#}, ^{*}—C(R^{V21}R^{V22})—C(R^{V21}R^{V22})—NR^{N3}—^{#}, —C(R^{V21}R^{V22})—NR^{N3}—^{#}, ^{*}—C(O)—C(R^{V23}R^{V24})—^{#}, ^{*}—C(O)—C(R^{V23}R^{V24})—C(R^{V23}R^{V24})—^{#}, ^{*}—C(O)—NR^{N3}—^{#} and ^{*}—C(O)—O—^{#}, wherein “*-” and “-^{#}” indicate the attachment points of V² as indicated in Formula (W-d);
U² is selected from the group consisting of a bond, ^{*}—C(O)—⁺, and ^{*}—C(R^{U21}R^{U22})—⁺, wherein “^{*}-” and “-⁺” indicate the attachment points of U² as indicated in Formula (W-d);
wherein if V² is ^{*}—C(R^{V21}R^{V22})—^{#}, U² is not a bond;
R^{U21} and R^{U22} are each independently selected from the group consisting of hydrogen, C₁-C₆ alkyl, hydroxy-C₂-C₆ alkyl, halo-C₂-C₆ alkyl, amino-C₂-C₆ alkyl, cyano-C₂-C₆ alkyl, —C(O)NR^{B}R^{C}, —C(O)R^{D}, —C(O)OH, —C(O)OR^{D}, C₁-C₆ alkyl-C(O)OH, and C₁-C₆ alkyl-C(O)OR^{D};
R^{V21} and R^{V22} are each independently selected from the group consisting of hydrogen, C₁-C₆ alkyl, hydroxy-C₂-C₆ alkyl, halo-C₂-C₆ alkyl, amino-C₂-C₆ alkyl, cyano-C₂-C₆ alkyl, —C(O)NR^{B}R^{C}, —C(O)R^{D}, —C(O)OH, and —C(O)OR^{D}; and
R^{V23} and R^{V24} are each independently selected from the group consisting of hydrogen, C₁-C₆ alkyl, hydroxy-C₁-C₆ alkyl, halo-C₁-C₆ alkyl, amino-C₁-C₆ alkyl, cyano-C₁-C₆ alkyl, halo, cyano, —OR^{A}, —NR^{B}R^{C}, —NR^{B}C(O)R^{D}, —C(O)NR^{B}R^{C}, —C(O)R^{D}, —C(O)OH, —C(O)OR^{D}, —SR^{E}, —S(O)R^{D}, and —S(O)₂R^{D}.

52. The compound of claim 51, wherein W is represented by Formula (W-d-1), Formula (W-d-2), Formula (W-d-3), Formula (W-d-4), or Formula (W-d-5):

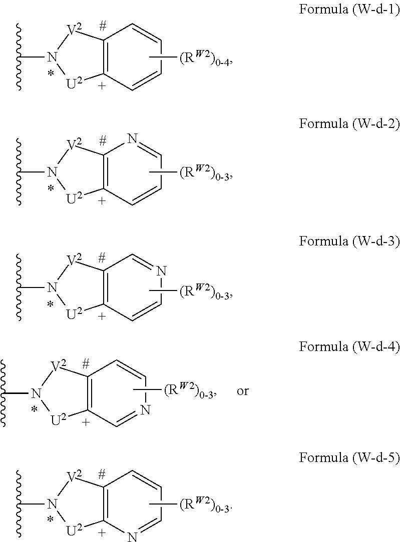

53. The compound of claim 52, wherein W is represented by Formula (W-d-1):

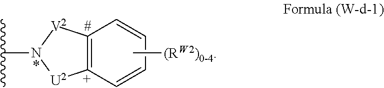

54. The compound of any one of claims 51-53, wherein V² is selected from the group consisting of ^{*}—C(R^{V21}R^{V22})—^{#}, ^{*}—C(R^{V21}R^{V22})—C(R^{V23}R^{V24})—^{#}, ^{*}—C(O)—C(R^{V23}R^{V24})—^{#} and ^{*}—C(R^{V21}R^{V22})—C(R^{V23}R^{V24})—C(R^{V23}R^{V24})—^{#}; wherein “^{*}-” and “-^{#}” indicate the attachment points of V² as indicated in Formula (W-d).

55. The compound of any one of claims 51-54, wherein each of R^{V21} and R^{V22} is independently selected from the group consisting of hydrogen and C₁-C₃ alkyl.

56. The compound of any one of claims 51-55, wherein each of R^{V21} and R^{V22} is hydrogen.

57. The compound of any one of claims 51-56, wherein each of R^{V23} and R^{V24} is independently selected from the group consisting of hydrogen, halo, C₁-C₃ alkyl, cyano, —OR^{A} and —NR^{B}R^{C}.

58. The compound of any one of claims 51-57, wherein each of R^{V23} and R^{V24} is hydrogen.

59. The compound of any one of claims 51-58, wherein U² is selected from the group consisting of a bond, ^{*}—C(O)—⁺, ^{*}—CH₂—⁺, and ^{*}—CH(CH₂CO₂H)—⁺, wherein “^{*}-” and “-⁺” indicate the attachment points of U² as indicated in Formula (W-d); and V² is selected from the group consisting of ^{*}—CH₂—^{#}, ^{*}—CH₂—CH₂—^{#}, ^{*}—C(O)—CH₂—^{#}, ^{*}—C(O)—NH—^{#}, ^{*}—CH₂—NH—^{#}, and ^{*}—CH₂—CH₂—CH₂—^{#}; wherein “^{*}-” and “-^{#}” indicate the attachment points of V² as indicated in Formula (W-d).

60. The compound of any one of claims 51-59, wherein W is an indoline, indolin-2-one, isoindoline, isoindolin-1-one, 1,2,3,4-tetrahydroquinoline, 1,2,3,4-tetrahydroisoquinoline, quinazoline-2,4(1H,3H)-dione, or 2,3-dihydroquinazolin-4(1H)-one moiety; wherein each of which is attached to L² through a nitrogen atom, and wherein each of which is optionally substituted on one or more available unsaturated carbon atoms with 1-4 R^{W2}, and wherein each R^{W2} is independently selected from the group consisting of C₁-C₆ alkyl, halo-C₁-C₆ alkyl, hydroxy-C₂-C₆ alkyl-O—, halo, cyano, and —OR^{A}.

61. The compound of any one of claims 51-60, wherein W is selected from the group consisting of: 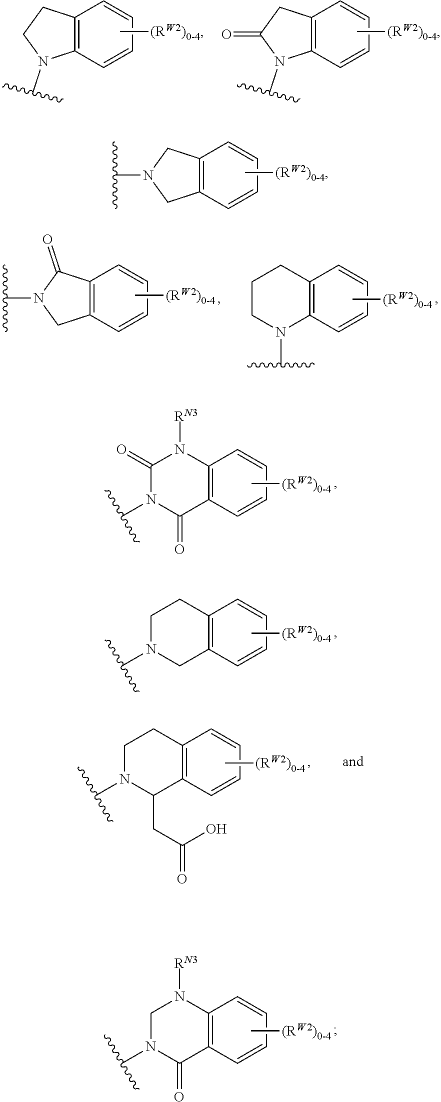
wherein R^{N3} is selected from the group consisting of hydrogen, C₁-C₆ alkyl, and hydroxy-C₂-C₆ alkyl.

62. The compound of any one of claims 1-61, wherein the compound of Formula (I) is a compound of Formula (I-a): 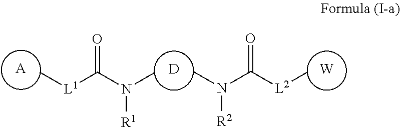
or a pharmaceutically acceptable salt, solvate, hydrate, tautomer, N-oxide, or stereoisomer thereof, wherein:
D is bicyclo[1.1.1]pentanyl or bicyclo[2.2.2]octanyl, each of which is optionally substituted with 1-4 R^{X} groups;
L¹ is selected from the group consisting of a bond and CH₂O—*, wherein “-*” indicates the attachment point to A;
L² is a bond;
R¹ is selected from the group consisting of hydrogen and CH₃;
R² is selected from the group consisting of hydrogen and CH₃;
A is phenyl, pyrazinyl or pyridyl, each of which is optionally substituted with 1-5 R^{Y}, groups;
W is a benzo[d][1,3]dioxole, 3,4-dihydro-2H-benzo[b][1,4]oxazine, chromane, chromene, chroman-4-one, chroman-4-ol, chroman-4-one oxime, 2H-benzo[b][1,4]oxazin-3(4H)-one, 2,3-dihydrobenzo[b][1,4]dioxine, indoline, 2,3-dihydrobenzofuran, benzofuran-3(2H)-one, 4H-chromen-4-ol or 4H-chromen-4-one moiety; wherein each of which is attached to L² through a carbon atom, and wherein each of which is optionally substituted on one or more available aromatic carbon atoms with 1-4 R^{W2} groups; and wherein 3,4-dihydro-2H-benzo[b][1,4]oxazine, 2H-benzo[b][1,4]oxazin-3(4H)-one, and indoline may be optionally substituted on an available nitrogen atom with hydrogen or CH₃;
each R^{W2} is independently selected from the group consisting of hydrogen, chloro, fluoro, CHF₂, CF₃, CH₃, CH₂CH₃, CH(CH₃)₂, OCH₃, OCHF₂, OCF₃, OCH₂CF₃, OCH(CH₃)₂, and CN; or
2 R^{W2} groups on adjacent carbons, together with the atoms to which they are attached form a 1,3-dioxolanyl ring, which is optionally substituted with 1-2 fluorine atoms;
each R^{X} is independently fluoro, oxo, OH, OCH₃, C(O)OH, or C(O)OCH₃; and
each R^{Y} is independently chloro, fluoro, CHF₂, CF₃, CH₃, CH₂CH₃, CH(CH₃)₂, OCH₃, OCHF₂, OCF₃, OCH₂CF₃, OCH(CH₃)₂, or CN; or
2 R^{Y} groups on adjacent atoms, together with the atoms to which they are attached form a 1,3-dioxolanyl ring, which is optionally substituted with 1-2 fluorine atoms.

63. The compound of any one of claims 1-61, wherein the compound of Formula (I) is a compound of Formula (I-b): 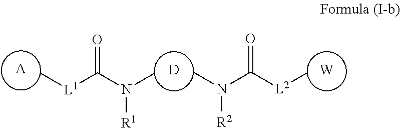
or a pharmaceutically acceptable salt, solvate, hydrate, tautomer, N-oxide, or stereoisomer thereof, wherein:
D is bicyclo[1.1.1]pentanyl or bicyclo[2.2.2]octanyl, each of which is optionally substituted with 1-4 R^{X} groups;
L¹ is selected from the group consisting of a bond and CH₂O—*, wherein “-*” indicates the attachment point to A;
L² is CH₂—*, wherein “-*” indicates the attachment point to W;
R¹ is selected from the group consisting of hydrogen and CH₃;
R² is selected from the group consisting of hydrogen and CH₃;
A is phenyl, pyrazinyl or pyridyl, each of which is optionally substituted with 1-5 R^{Y} groups;
W is an indoline or tetrahydroisoquinoline moiety; wherein indoline or tetrahydroisoquinoline is attached to L² through a nitrogen atom, and wherein indoline or tetrahydroisoquinoline is optionally substituted on one or more available unsaturated carbon atoms with 1-4 R^{W2} groups;
each R^{W2} is independently selected from the group consisting of hydrogen, chloro, fluoro, CHF₂, CF₃, CH₃, CH₂CH₃, CH(CH₃)₂, OCH₃, OCHF₂, OCF₃, OCH₂CF₃, OCH(CH₃)₂, and CN; or
2 R^{W2} groups on adjacent carbons, together with the atoms to which they are attached form a 1,3-dioxolanyl ring, which is optionally substituted with 1-2 fluorine atoms;
each R^{X} is independently fluoro, oxo, OH, OCH₃, C(O)OH, or C(O)OCH₃; and
each R^{Y} is independently chloro, fluoro, CHF₂, CF₃, CH₃, CH₂CH₃, CH(CH₃)₂, OCH₃, OCHF₂, OCF₃, OCH₂CF₃, OCH(CH₃)₂, or CN; or
2 R^{Y} groups on adjacent atoms, together with the atoms to which they are attached form a 1,3-dioxolanyl ring, which is optionally substituted with 1-2 fluorine atoms.

64. The compound of any one of claims 1-63, wherein the compound of Formula (I) is a compound of Formula (I-e-1), Formula (I-e-2), Formula (I-e-3), Formula (I-e-4), Formula (I-e-5), Formula (I-e-6), Formula (I-e-7), Formula (I-e-8), Formula (I-e-9), Formula (I-e-10), Formula (I-e-11), Formula (I-e-12), Formula (I-e-13), Formula (I-e-14), Formula (I-e-15), Formula (I-e-16), or Formula (I-e-17). 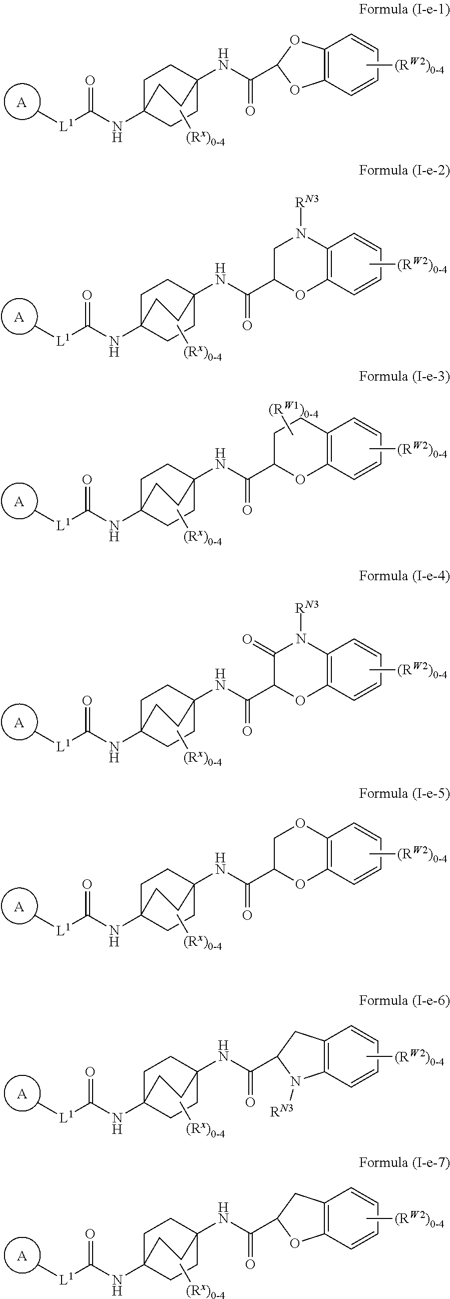 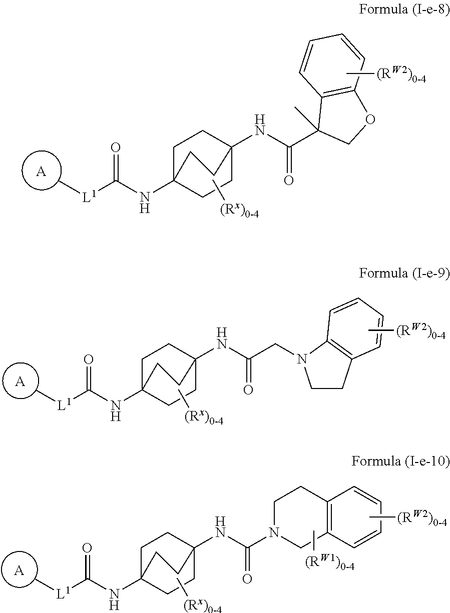 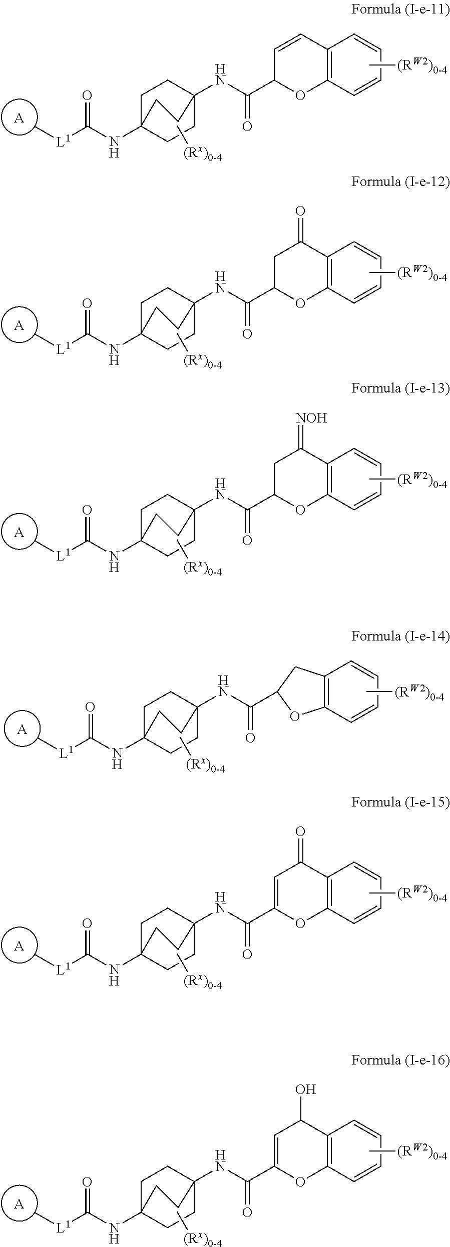 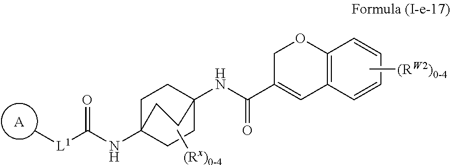
or a pharmaceutically acceptable salt, solvate, hydrate, tautomer, N-oxide, or stereoisomer thereof.

65. The compound of any one of claims 1-63, wherein the compound of Formula (I) is a compound of Formula (I-f-1), Formula (I-f-2), Formula (I-f-3), Formula (I-f-4), Formula (I-f-5), Formula (I-f-6), Formula (I-f-7), Formula (I-f-8), Formula (I-f-9), Formula (I-f-10), Formula (I-f-11), Formula (I-f-12), Formula (I-f-13), Formula (I-f-14), Formula (I-f-15), Formula (I-f-16), or Formula (I-f-17): 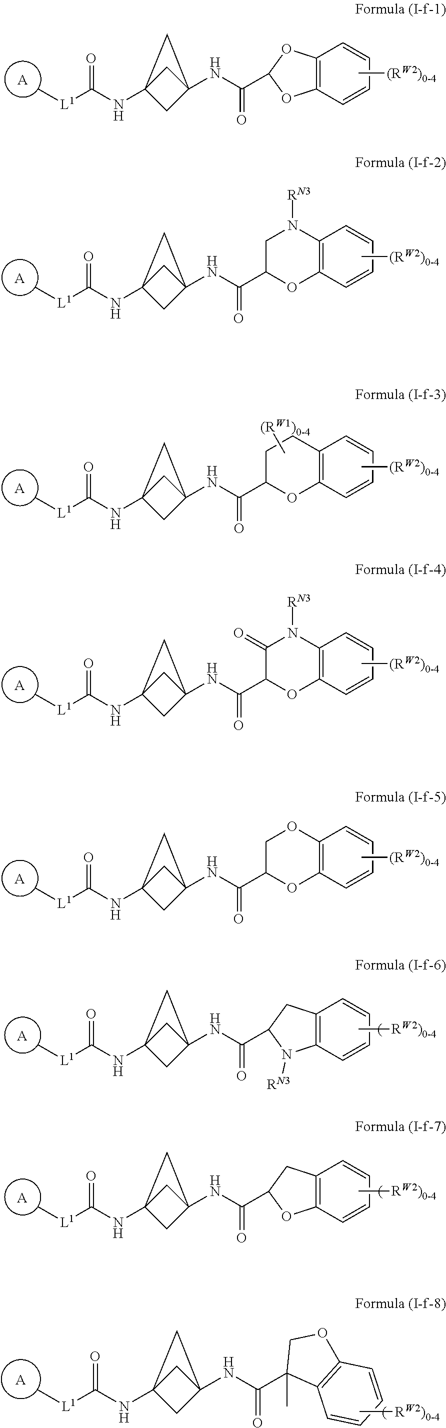 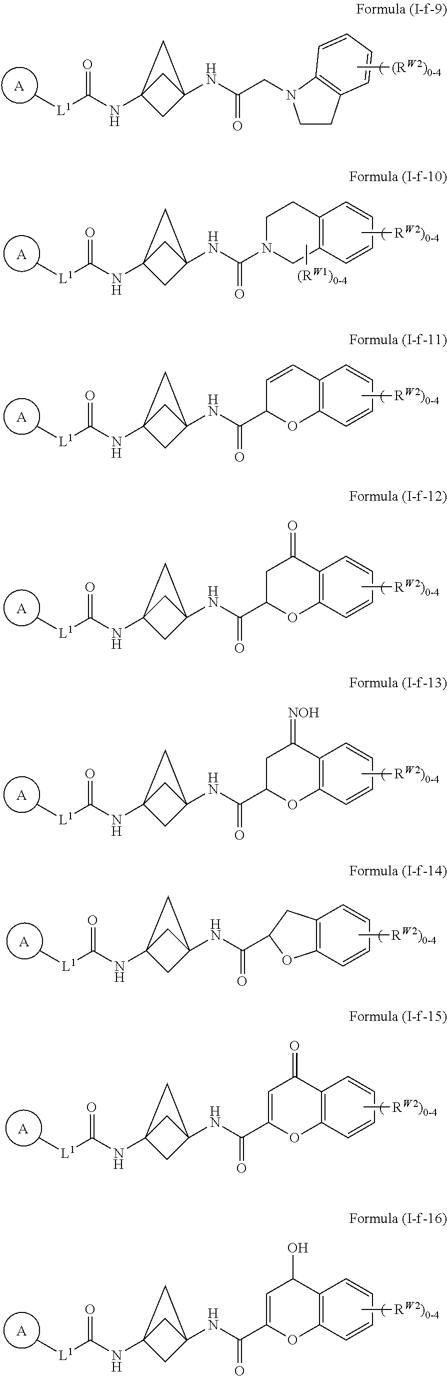 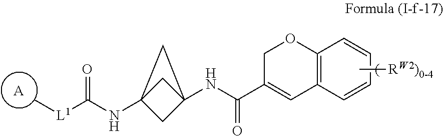
or a pharmaceutically acceptable salt, solvate, hydrate, tautomer, N-oxide, or stereoisomer thereof.

66. A compound selected from the group consisting of: 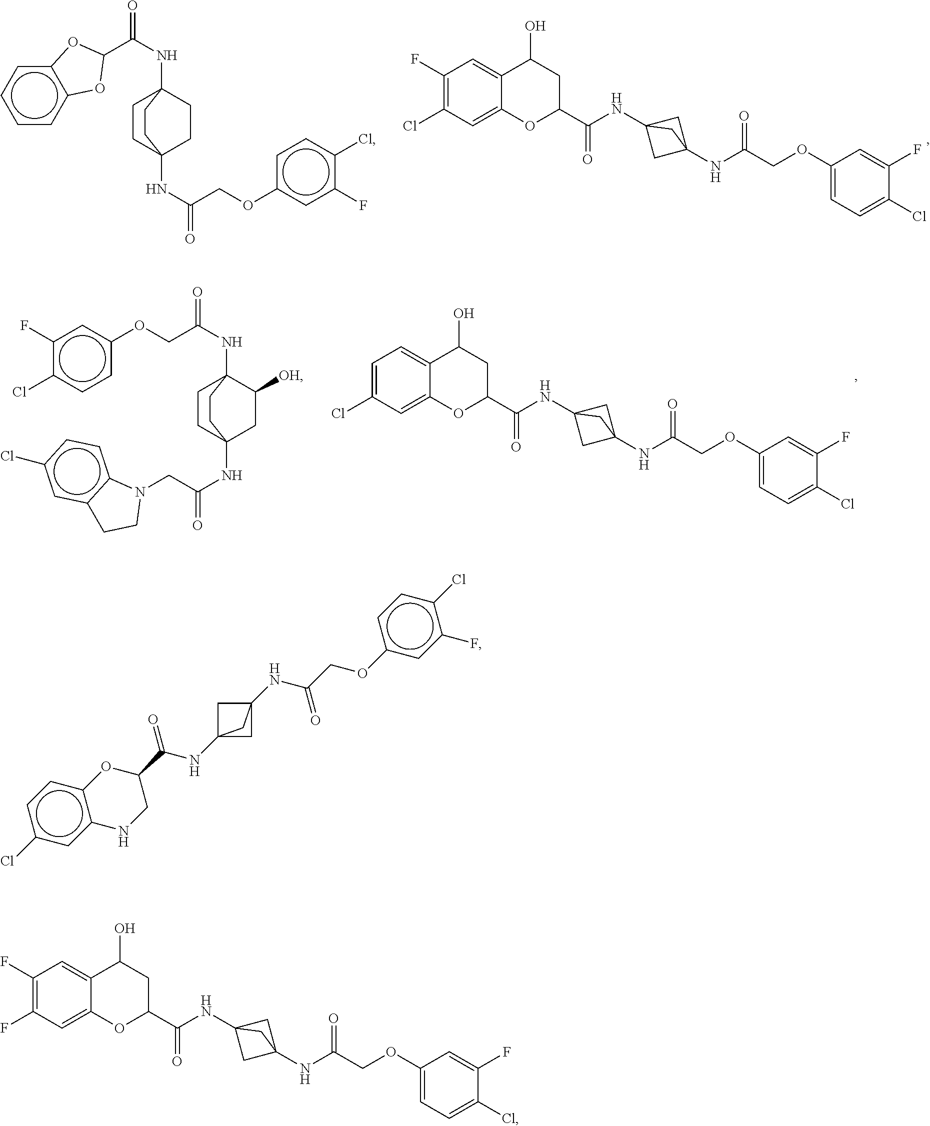 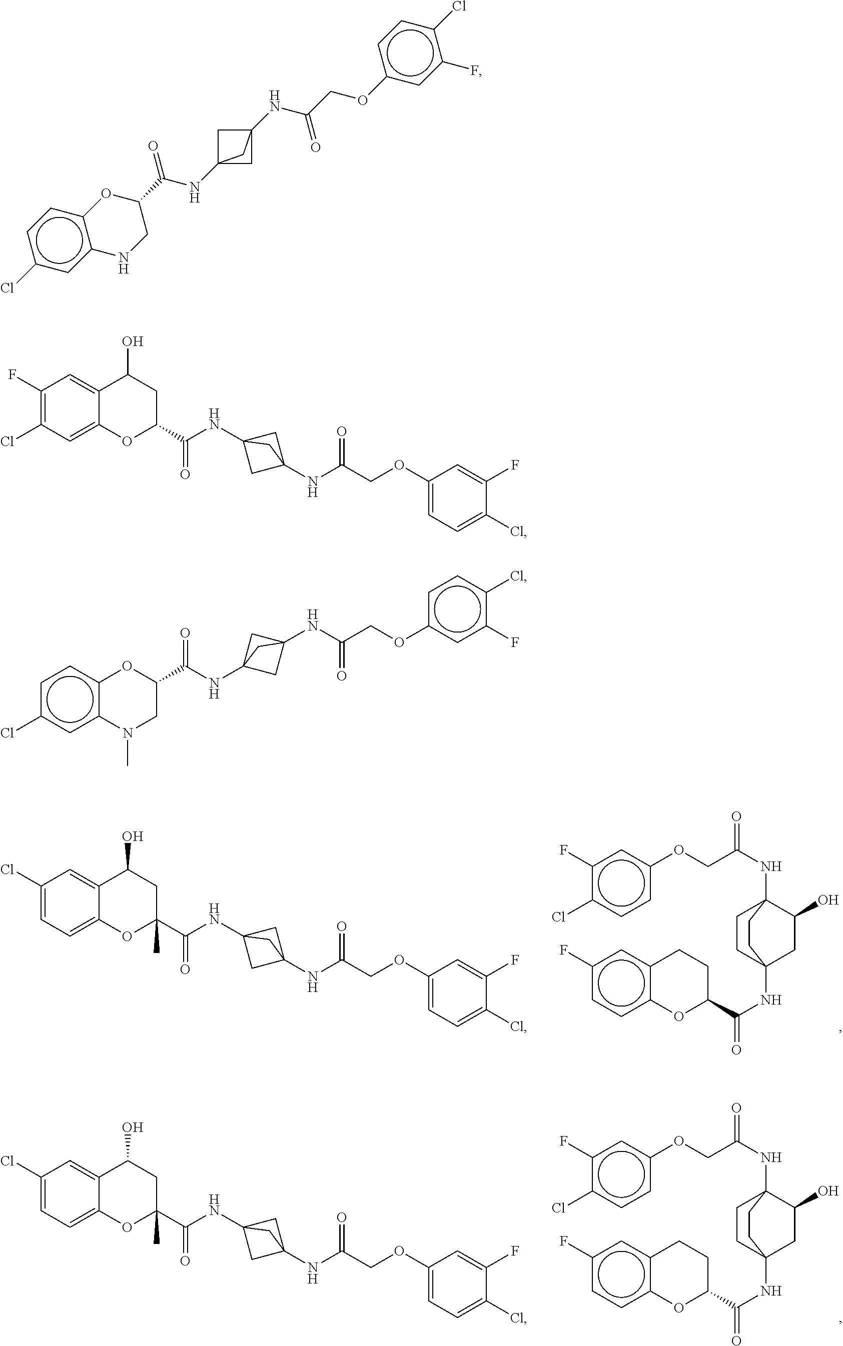 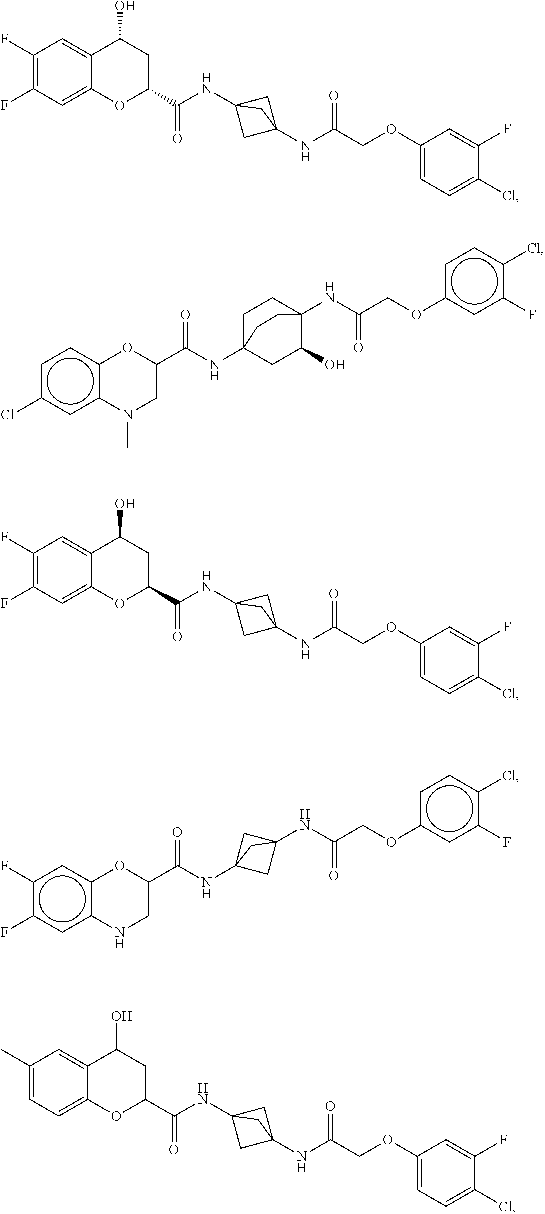 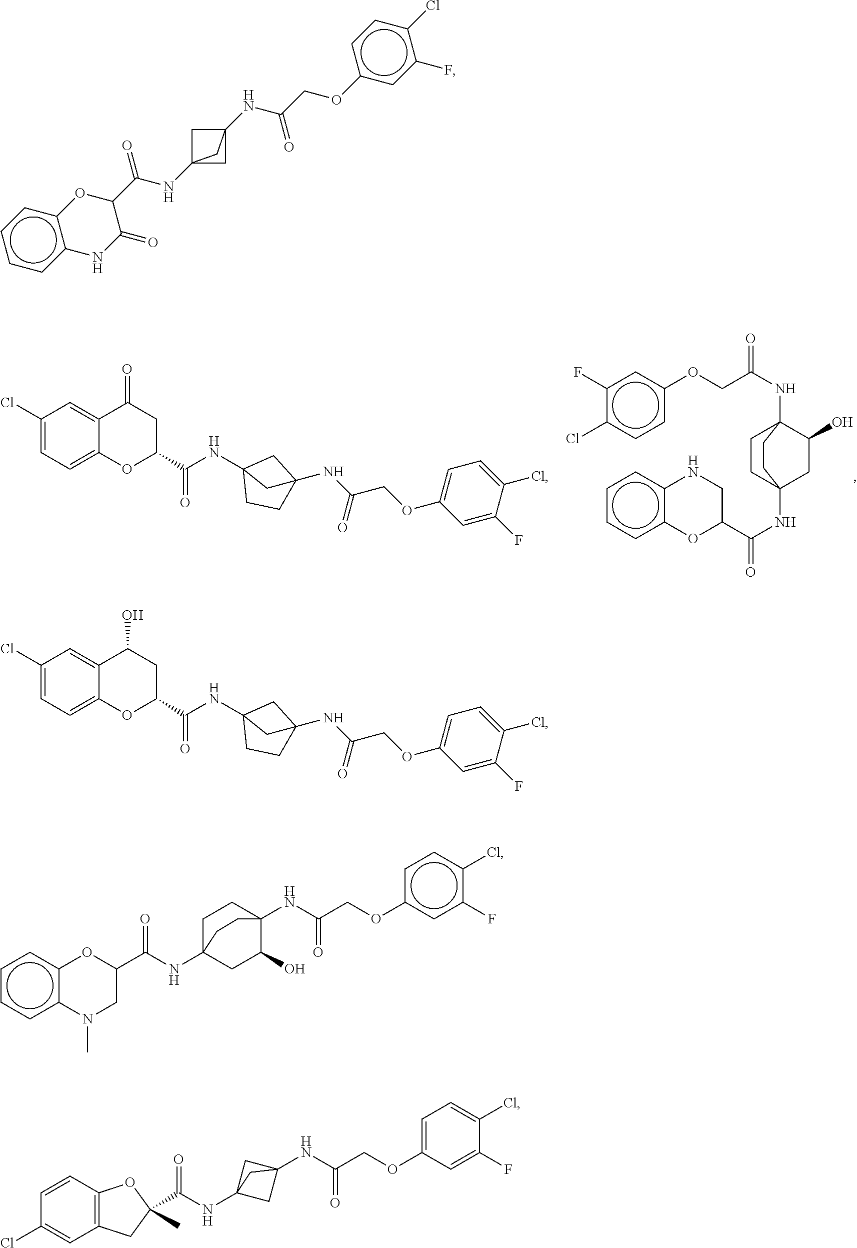 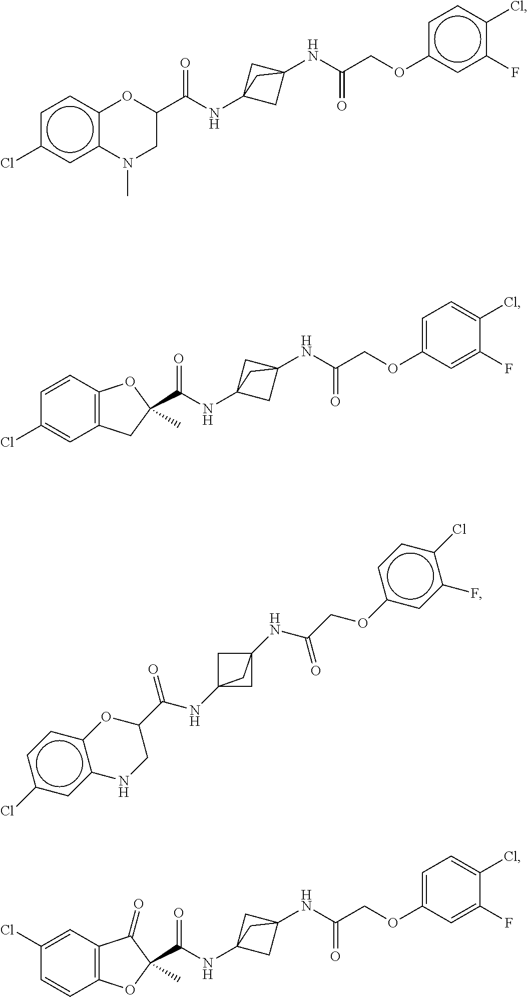 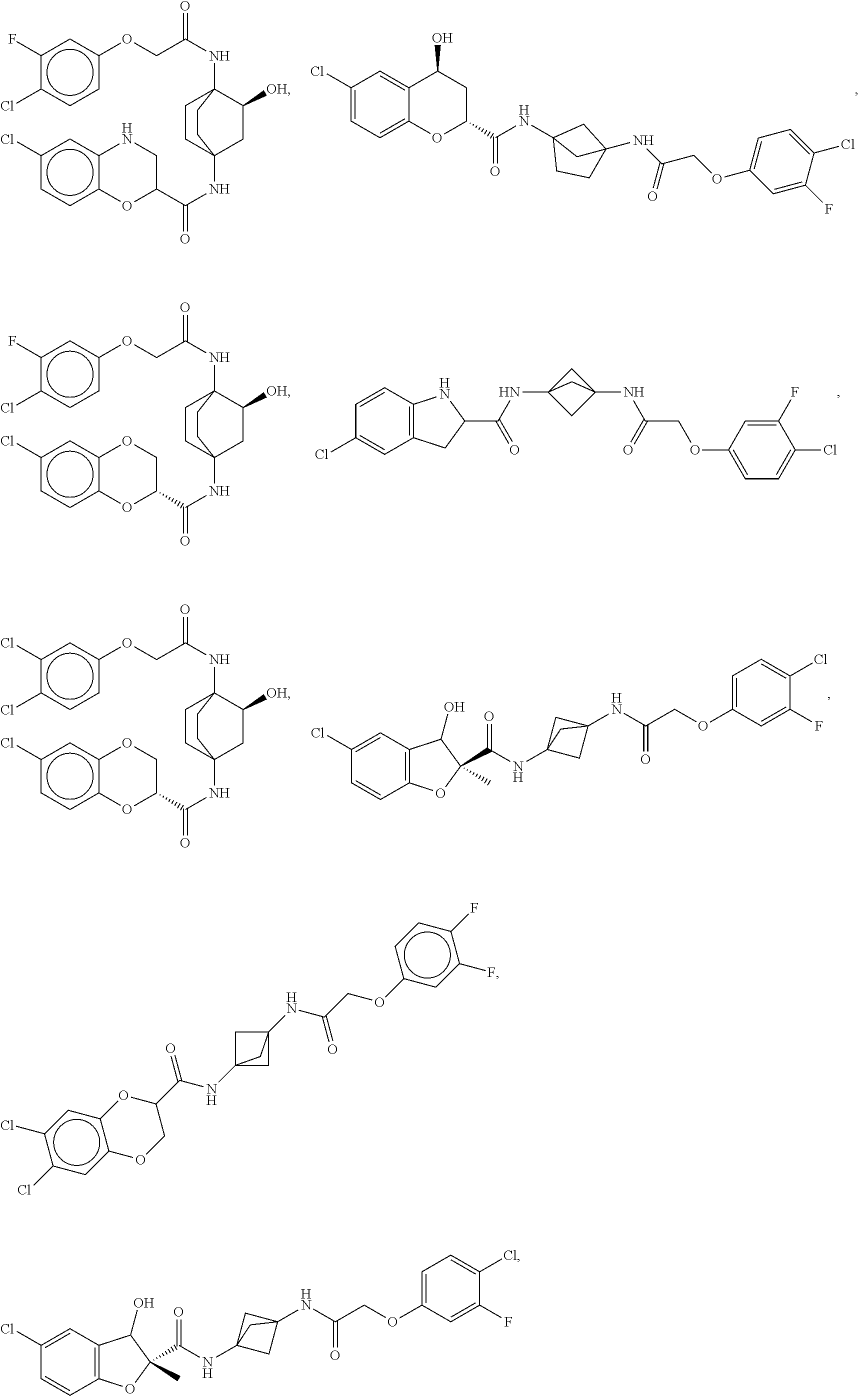 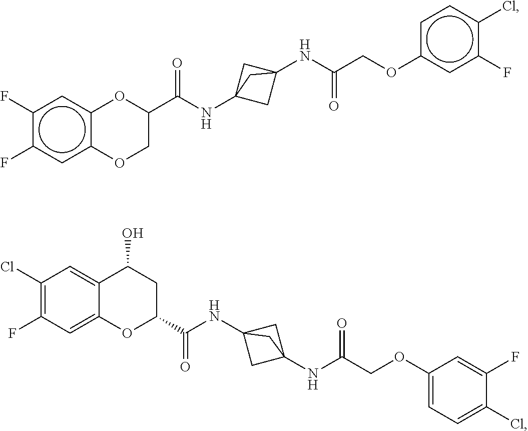 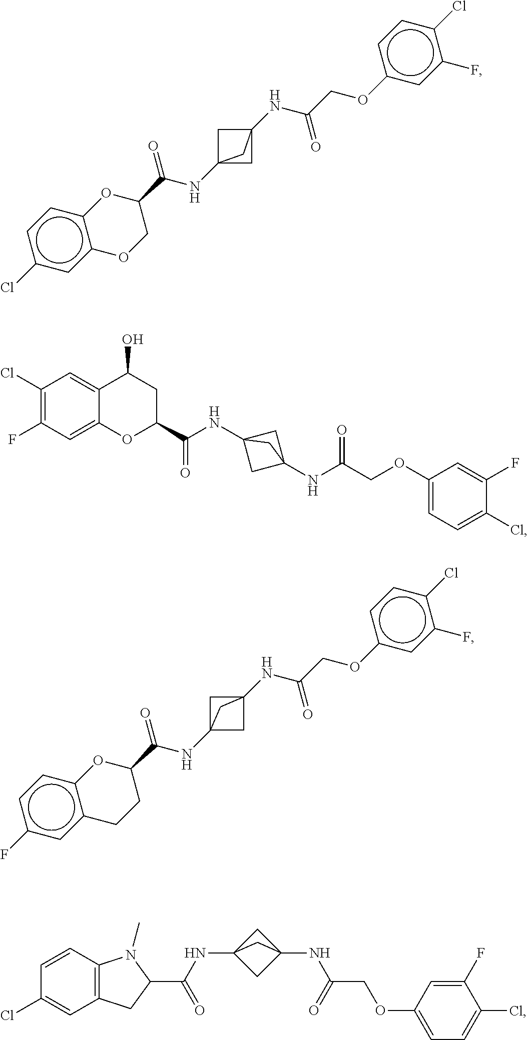 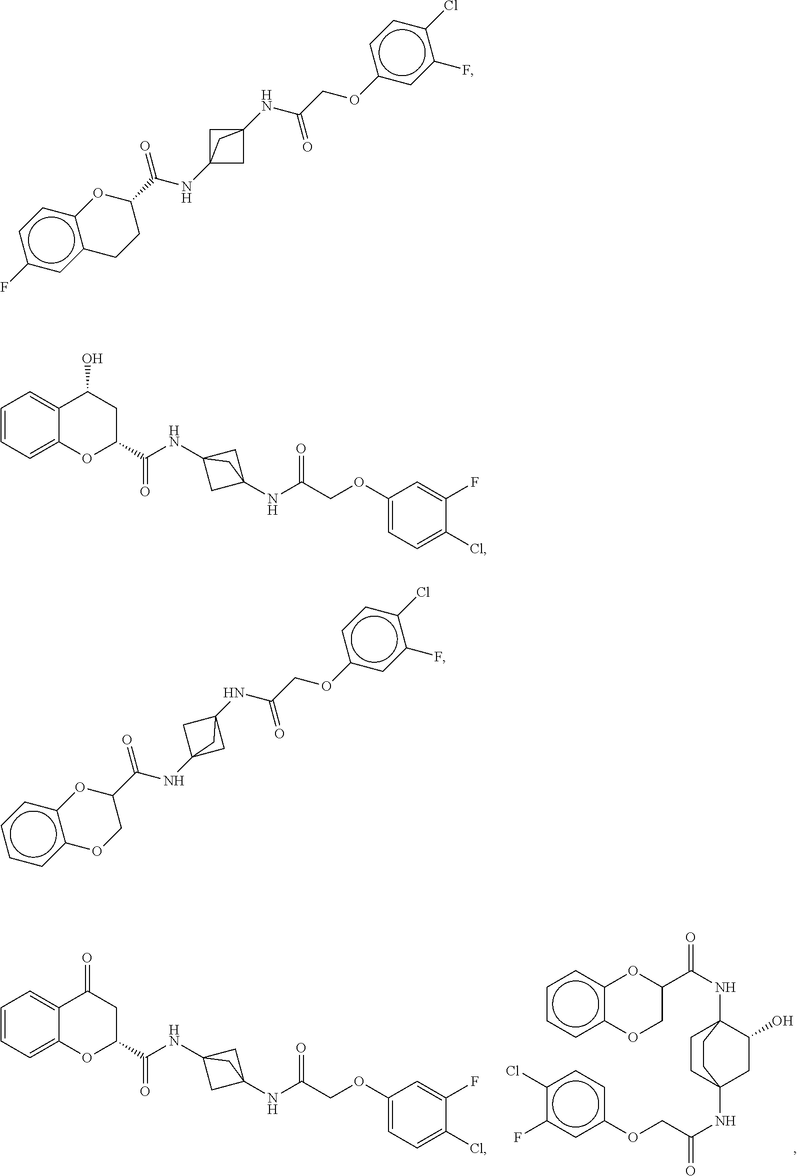 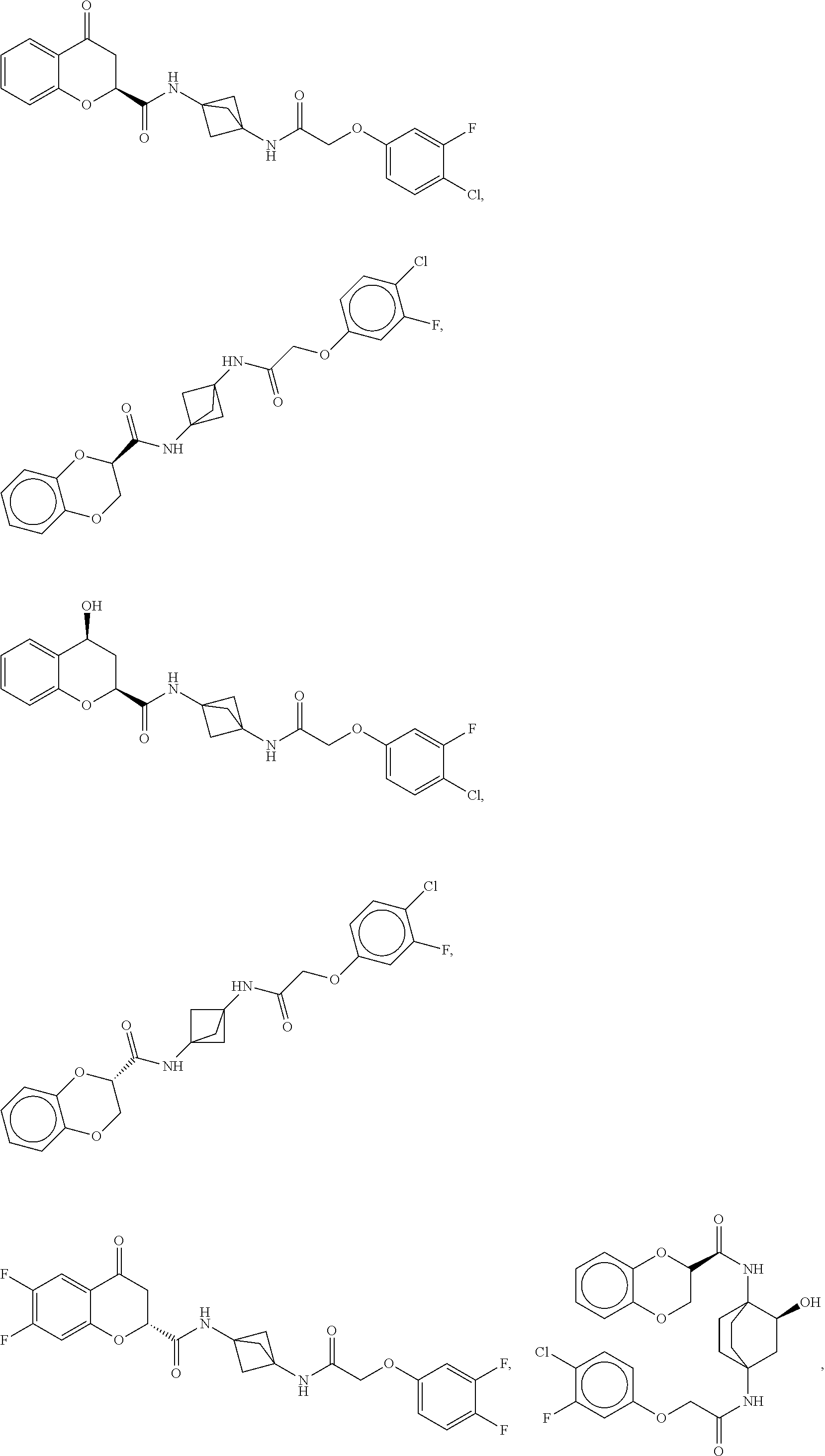 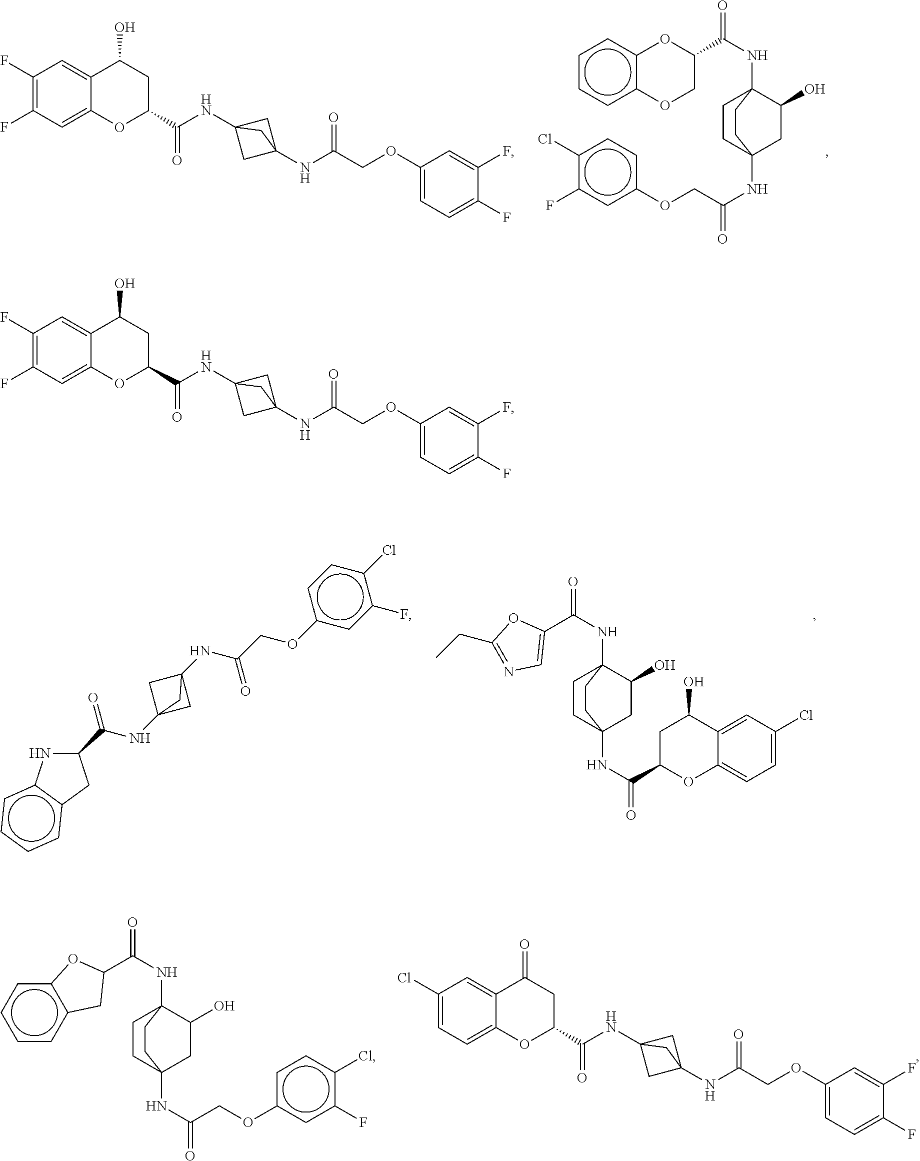 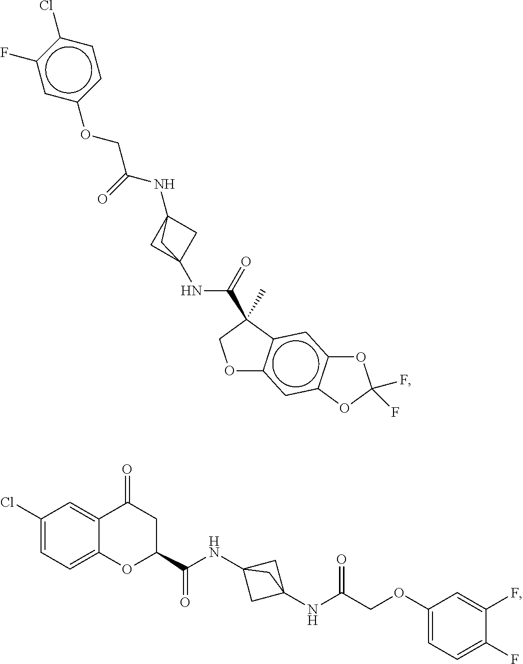 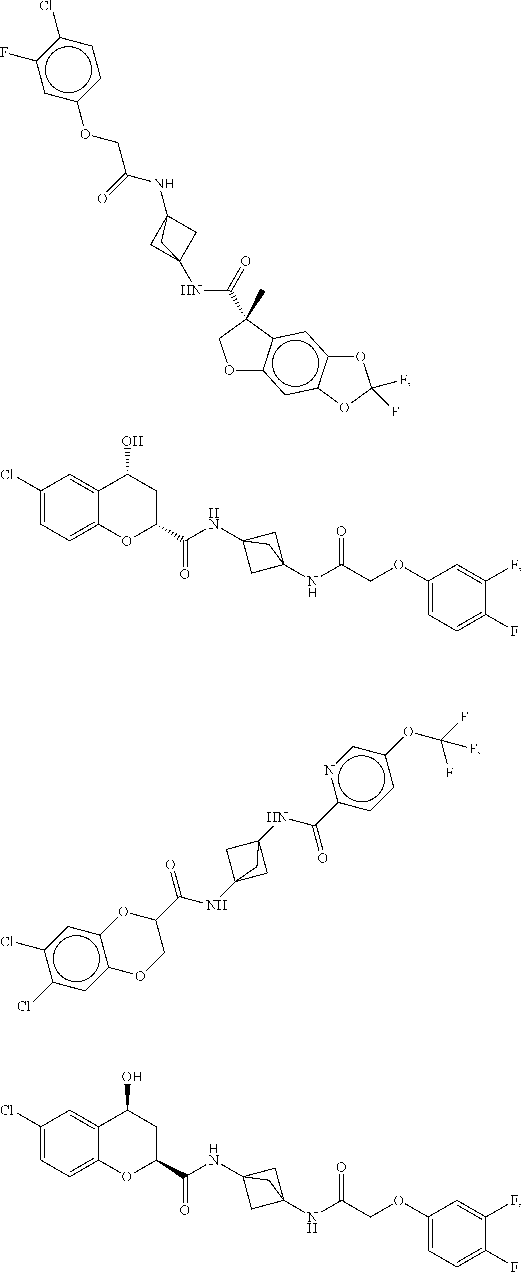 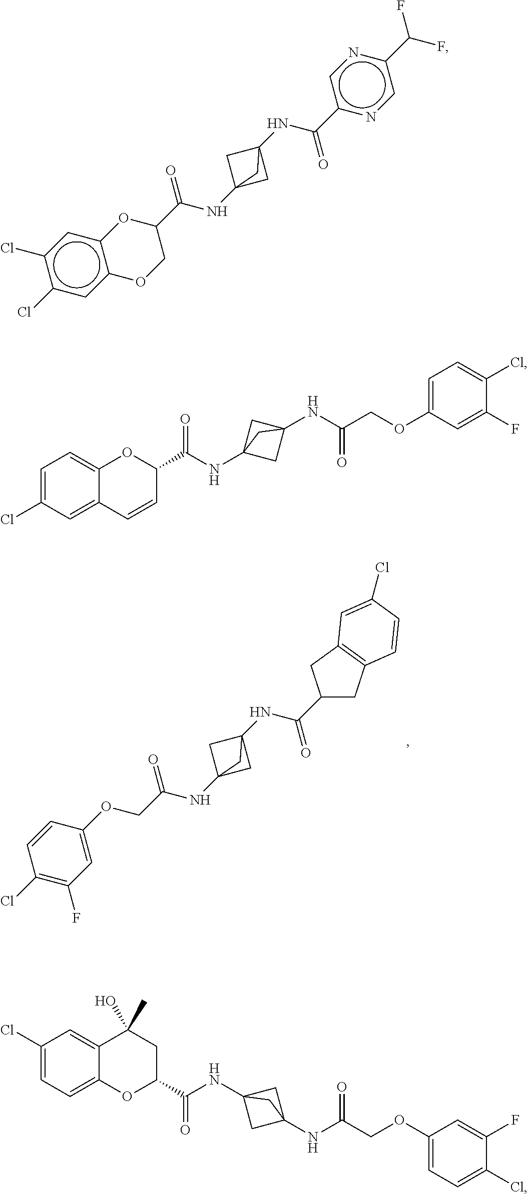 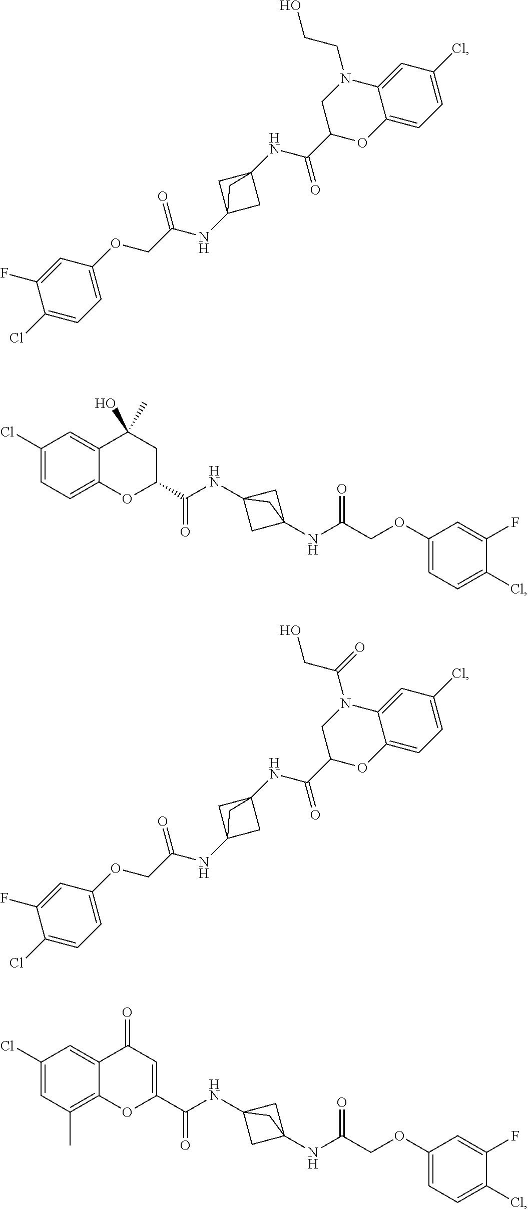 [SMILES: O=C(COc1ccc(Cl)c(F)c1)NC12CC(NC(=O)C3CN(CCO)c4cc(Cl)ccc4O3)(C1)C2 ; C[C@]1(O)C[C@H](C(=O)NC23CC(NC(=O)COc4ccc(Cl)c(F)c4)(C2)C3)Oc2ccc(Cl)cc21 ; O=C(COc1ccc(Cl)c(F)c1)NC12CC(NC(=O)C3CN(C(=O)CO)c4cc(Cl)ccc4O3)(C1)C2 ; Cc1cc(Cl)cc2c(=O)cc(C(=O)NC34CC(NC(=O)COc5ccc(Cl)c(F)c5)(C3)C4)oc12] 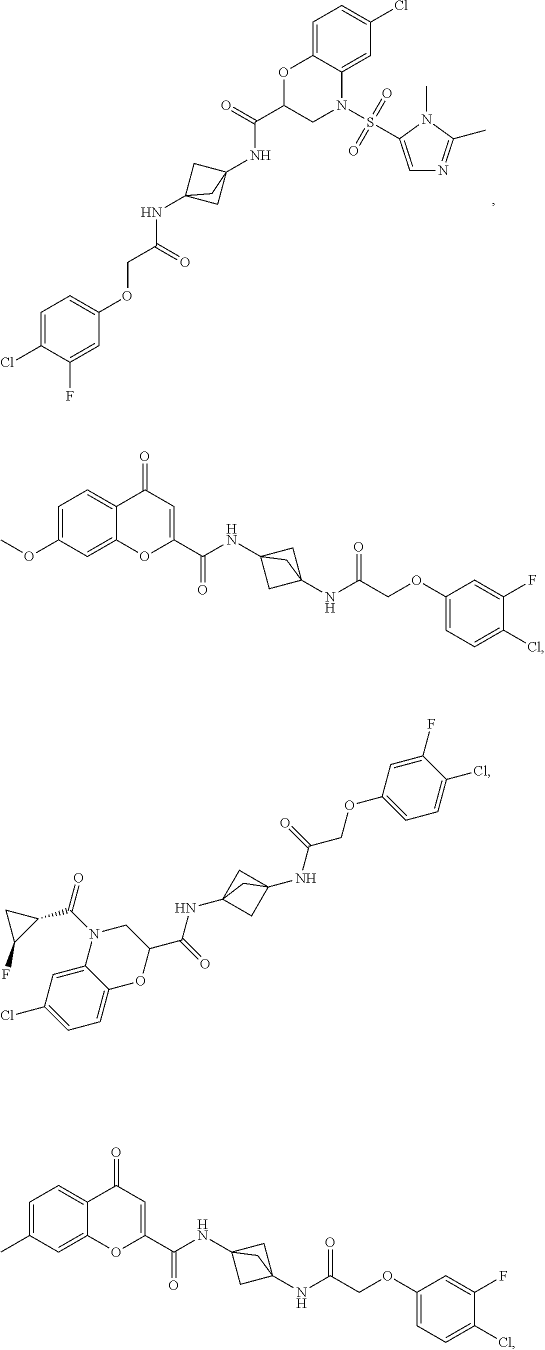 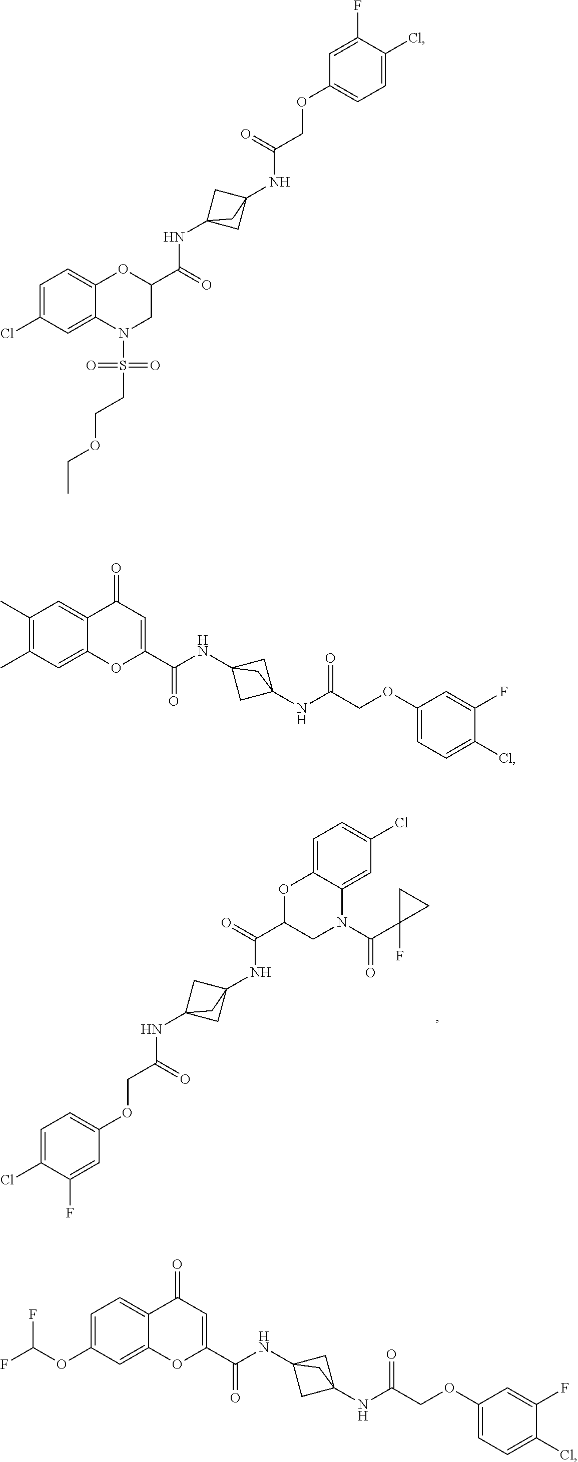 [SMILES: CCOCCS(=O)(=O)N1CC(C(=O)NC23CC(NC(=O)COc4ccc(Cl)c(F)c4)(C2)C3)Oc2ccc(Cl)cc21 ; Cc1cc2oc(C(=O)NC34CC(NC(=O)COc5ccc(Cl)c(F)c5)(C3)C4)cc(=O)c2cc1C ; O=C(COc1ccc(Cl)c(F)c1)NC12CC(NC(=O)C3CN(C(=O)C4(F)CC4)c4cc(Cl)ccc4O3)(C1)C2 ; O=C(COc1ccc(Cl)c(F)c1)NC12CC(NC(=O)c3cc(=O)c4ccc(OC(F)F)cc4o3)(C1)C2] 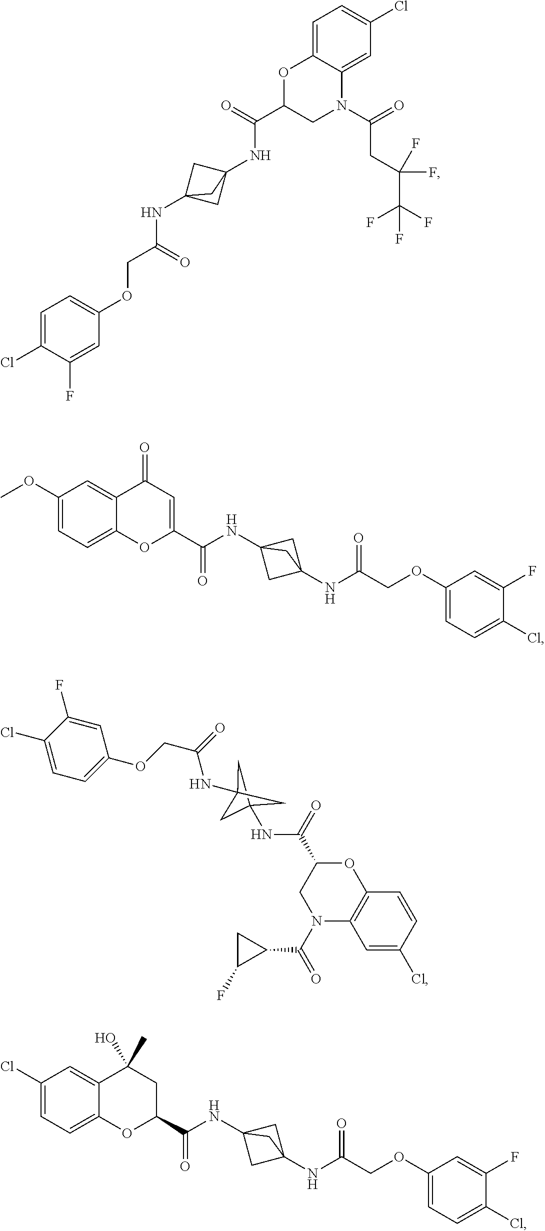 [SMILES: O=C(COc1ccc(Cl)c(F)c1)NC12CC(NC(=O)C3CN(C(=O)CC(F)(F)C(F)(F)F)c4cc(Cl)ccc4O3)(C1)C2 ; COc1ccc2oc(C(=O)NC34CC(NC(=O)COc5ccc(Cl)c(F)c5)(C3)C4)cc(=O)c2c1 ; O=C(COc1ccc(Cl)c(F)c1)NC12CC(NC(=O)[C@H]3CN(C(=O)[C@H]4C[C@H]4F)c4cc(Cl)ccc4O3)(C1)C2 ; C[C@@]1(O)C[C@@H](C(=O)NC23CC(NC(=O)COc4ccc(Cl)c(F)c4)(C2)C3)Oc2ccc(Cl)cc21] 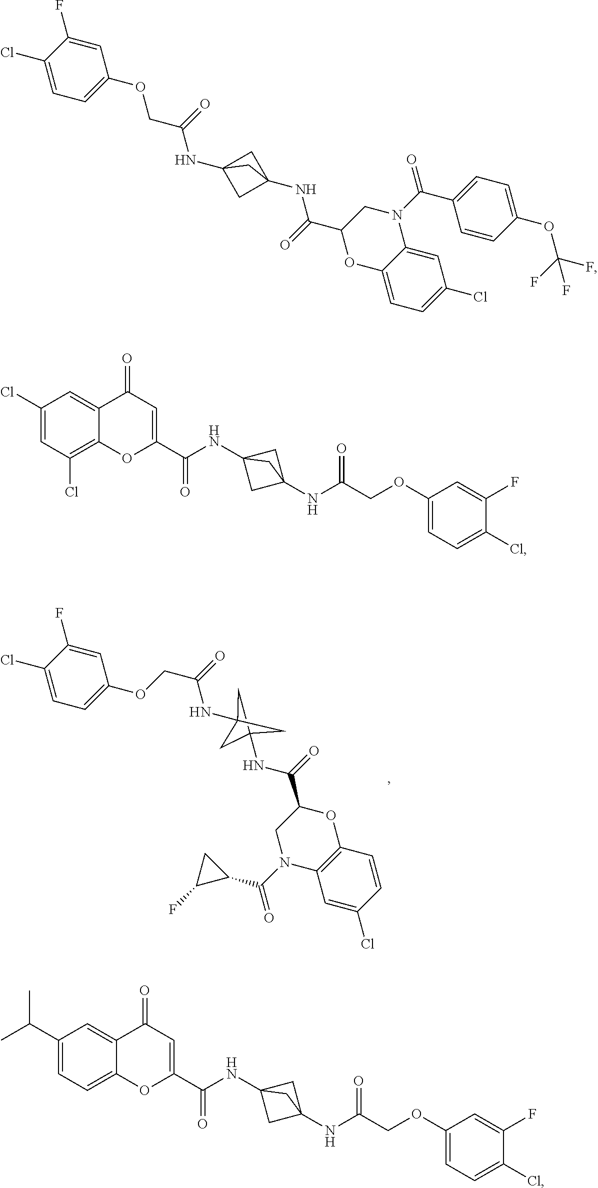 [SMILES: O=C(COc1ccc(Cl)c(F)c1)NC12CC(NC(=O)C3CN(C(=O)c4ccc(OC(F)(F)F)cc4)c4cc(Cl)ccc4O3)(C1)C2 ; O=C(COc1ccc(Cl)c(F)c1)NC12CC(NC(=O)c3cc(=O)c4cc(Cl)cc(Cl)c4o3)(C1)C2 ; O=C(COc1ccc(Cl)c(F)c1)NC12CC(NC(=O)[C@@H]3CN(C(=O)[C@H]4C[C@H]4F)c4cc(Cl)ccc4O3)(C1)C2 ; CC(C)c1ccc2oc(C(=O)NC34CC(NC(=O)COc5ccc(Cl)c(F)c5)(C3)C4)cc(=O)c2c1] 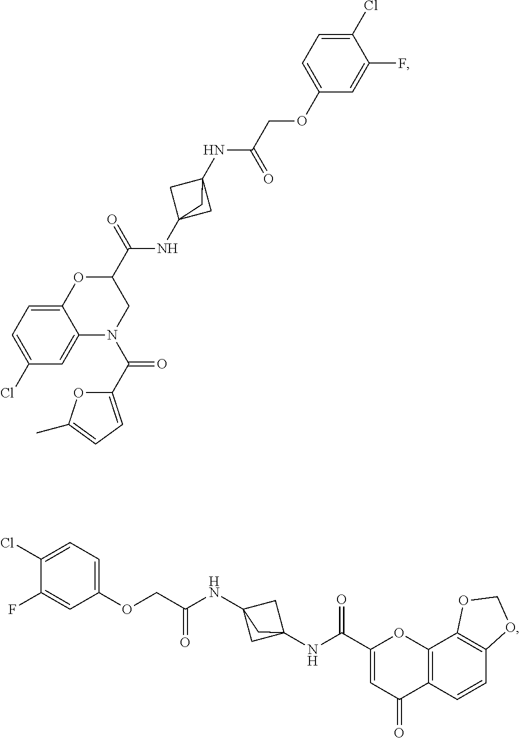 [SMILES: Cc1ccc(C(=O)N2CC(C(=O)NC34CC(NC(=O)COc5ccc(Cl)c(F)c5)(C3)C4)Oc3ccc(Cl)cc32)o1 ; O=C(COc1ccc(Cl)c(F)c1)NC12CC(NC(=O)c3cc(=O)c4ccc5c(c4o3)OCO5)(C1)C2] 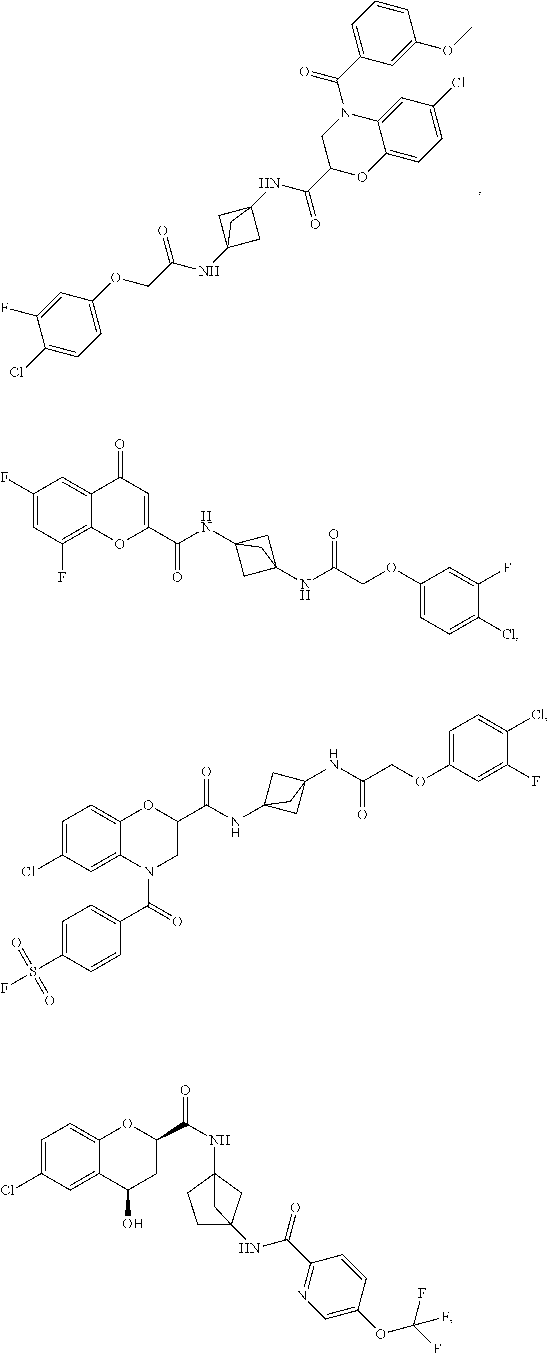 [SMILES: COc1cccc(C(=O)N2CC(C(=O)NC34CC(NC(=O)COc5ccc(Cl)c(F)c5)(C3)C4)Oc3ccc(Cl)cc32)c1 ; O=C(COc1ccc(Cl)c(F)c1)NC12CC(NC(=O)c3cc(=O)c4cc(F)cc(F)c4o3)(C1)C2 ; O=C(COc1ccc(Cl)c(F)c1)NC12CC(NC(=O)C3CN(C(=O)c4ccc(S(=O)(=O)F)cc4)c4cc(Cl)ccc4O3)(C1)C2 ; O=C(NC12CCC(NC(=O)[C@H]3C[C@@H](O)c4cc(Cl)ccc4O3)(C1)C2)c1ccc(OC(F)(F)F)cn1] 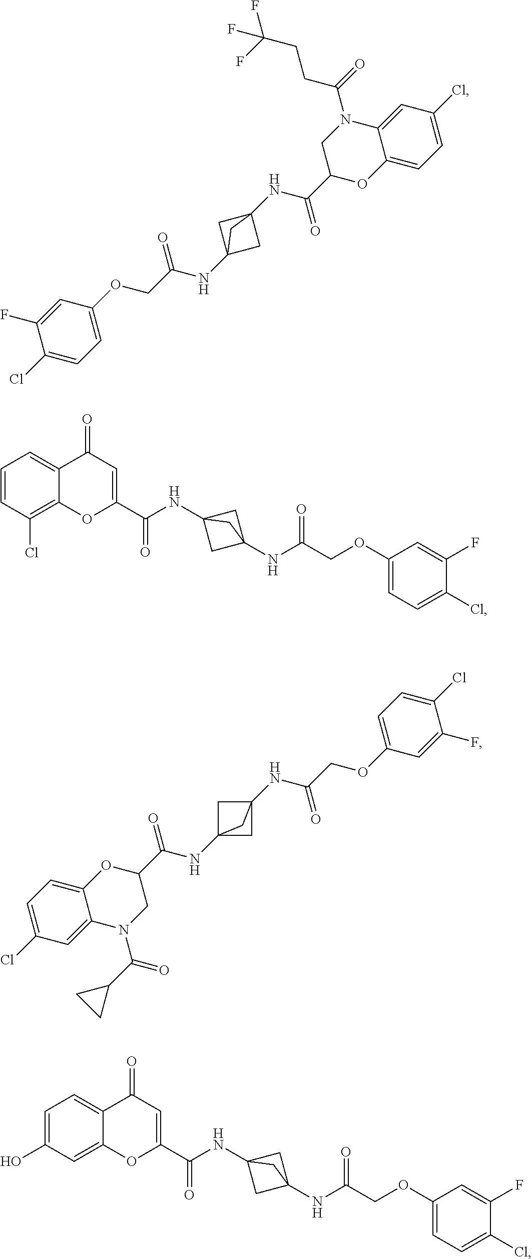 [SMILES: O=C(COc1ccc(Cl)c(F)c1)NC12CC(NC(=O)C3CN(C(=O)CCC(F)(F)F)c4cc(Cl)ccc4O3)(C1)C2 ; O=C(COc1ccc(Cl)c(F)c1)NC12CC(NC(=O)c3cc(=O)c4cccc(Cl)c4o3)(C1)C2 ; O=C(COc1ccc(Cl)c(F)c1)NC12CC(NC(=O)C3CN(C(=O)C4CC4)c4cc(Cl)ccc4O3)(C1)C2 ; O=C(COc1ccc(Cl)c(F)c1)NC12CC(NC(=O)c3cc(=O)c4ccc(O)cc4o3)(C1)C2] 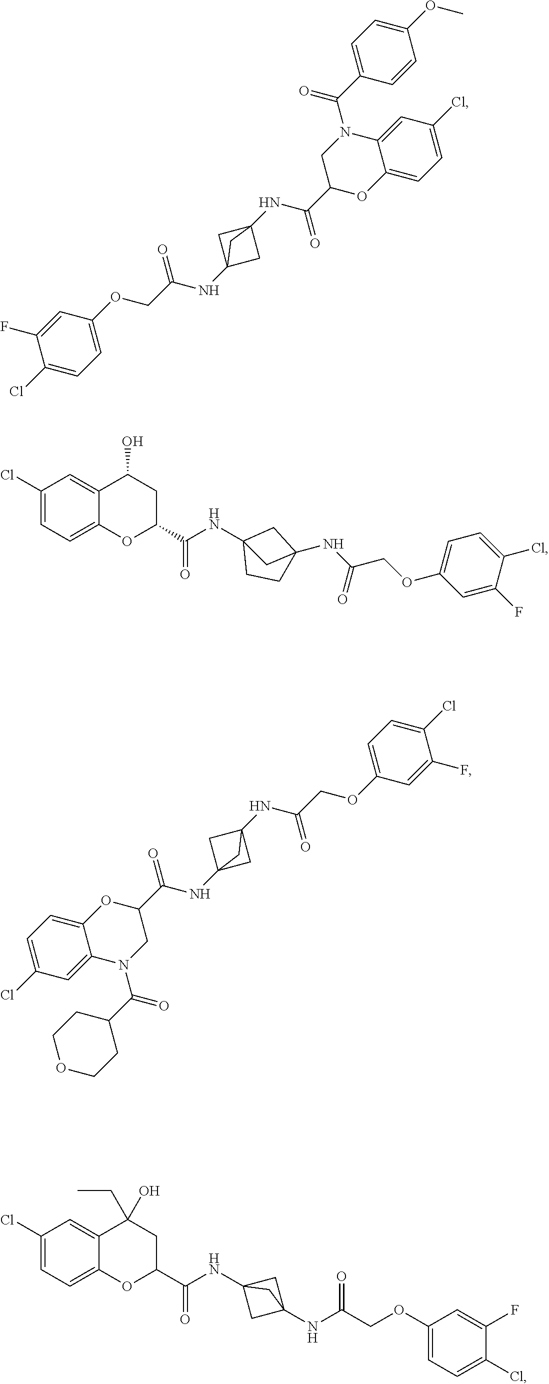 [SMILES: COc1ccc(C(=O)N2CC(C(=O)NC34CC(NC(=O)COc5ccc(Cl)c(F)c5)(C3)C4)Oc3ccc(Cl)cc32)cc1 ; O=C(COc1ccc(Cl)c(F)c1)NC12CCC(NC(=O)[C@H]3C[C@@H](O)c4cc(Cl)ccc4O3)(C1)C2 ; O=C(COc1ccc(Cl)c(F)c1)NC12CC(NC(=O)C3CN(C(=O)C4CCOCC4)c4cc(Cl)ccc4O3)(C1)C2 ; CCC1(O)CC(C(=O)NC23CC(NC(=O)COc4ccc(Cl)c(F)c4)(C2)C3)Oc2ccc(Cl)cc21] 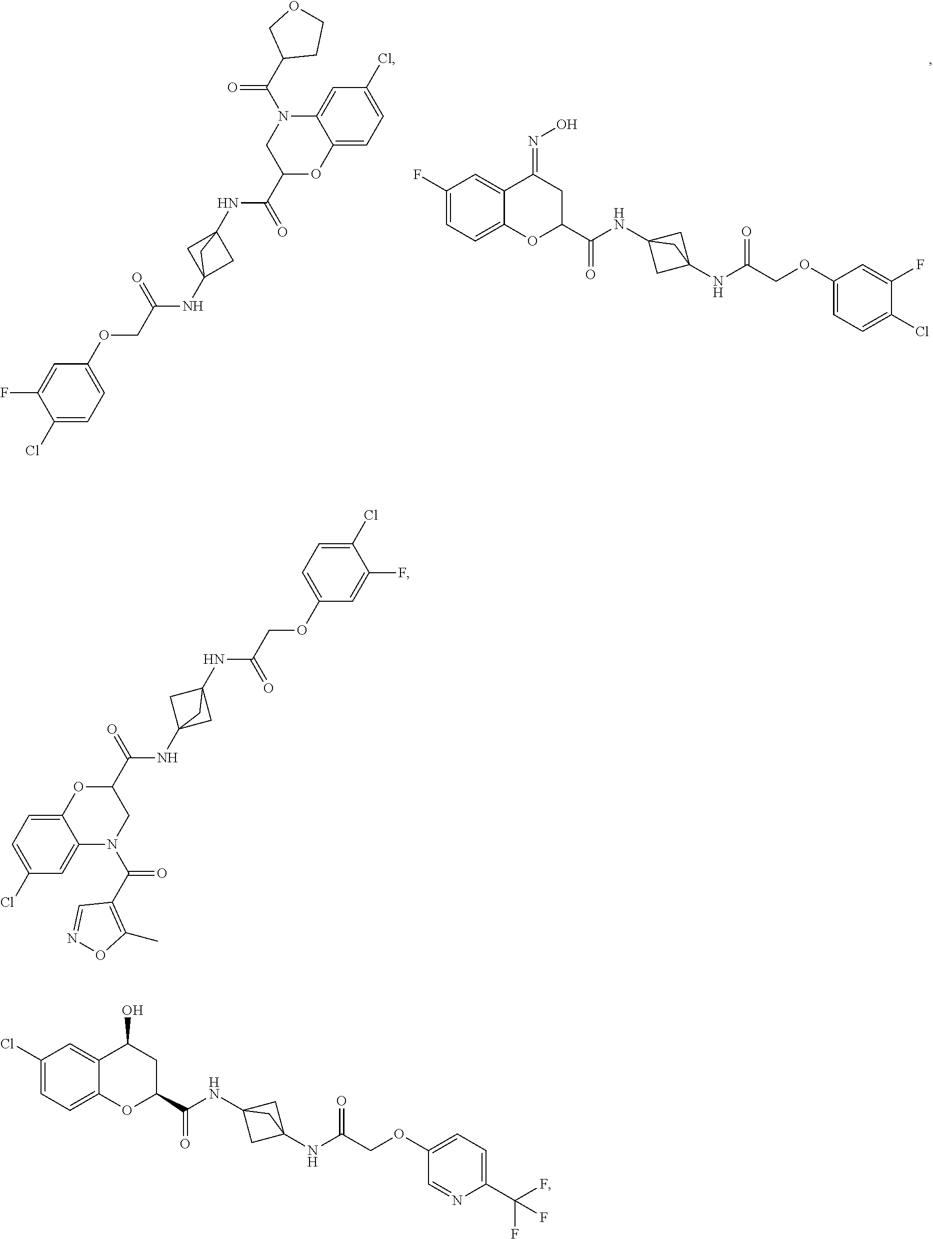 [SMILES: O=C(COc1ccc(Cl)c(F)c1)NC12CC(NC(=O)C3CN(C(=O)C4CCOC4)c4cc(Cl)ccc4O3)(C1)C2 ; O=C(COc1ccc(Cl)c(F)c1)NC12CC(NC(=O)C3C/C(=N\O)c4cc(F)ccc4O3)(C1)C2 ; Cc1oncc1C(=O)N1CC(C(=O)NC23CC(NC(=O)COc4ccc(Cl)c(F)c4)(C2)C3)Oc2ccc(Cl)cc21 ; O=C(COc1ccc(C(F)(F)F)nc1)NC12CC(NC(=O)[C@@H]3C[C@H](O)c4cc(Cl)ccc4O3)(C1)C2] 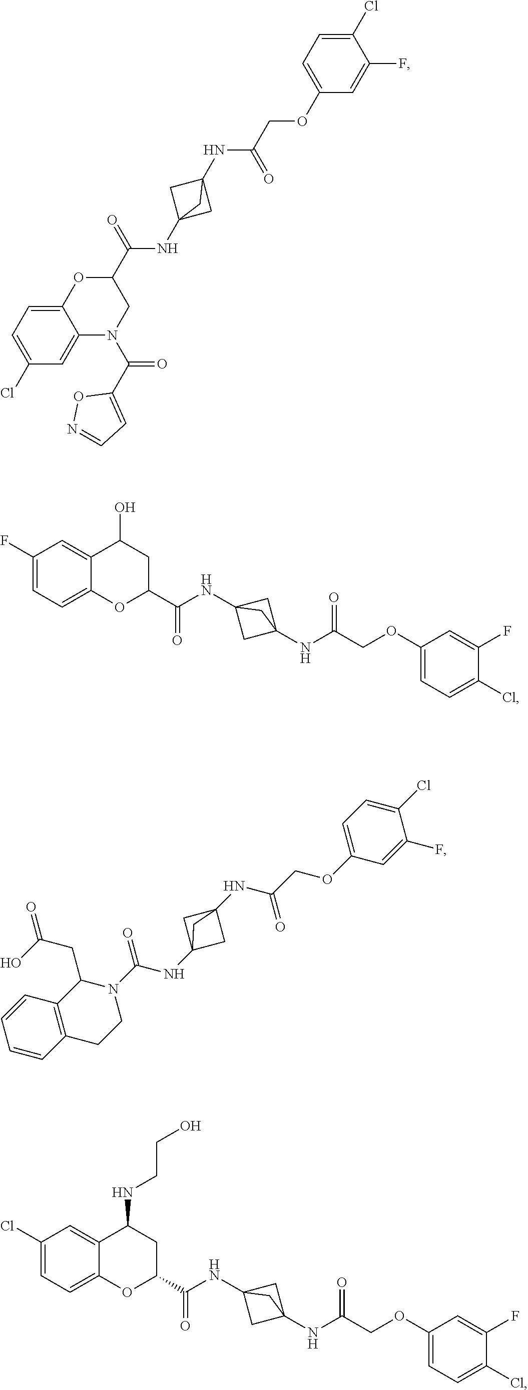 [SMILES: O=C(COc1ccc(Cl)c(F)c1)NC12CC(NC(=O)C3CN(C(=O)c4ccno4)c4cc(Cl)ccc4O3)(C1)C2 ; O=C(COc1ccc(Cl)c(F)c1)NC12CC(NC(=O)C3CC(O)c4cc(F)ccc4O3)(C1)C2 ; O=C(O)CC1c2ccccc2CCN1C(=O)NC12CC(NC(=O)COc3ccc(Cl)c(F)c3)(C1)C2 ; O=C(COc1ccc(Cl)c(F)c1)NC12CC(NC(=O)[C@H]3C[C@H](NCCO)c4cc(Cl)ccc4O3)(C1)C2] 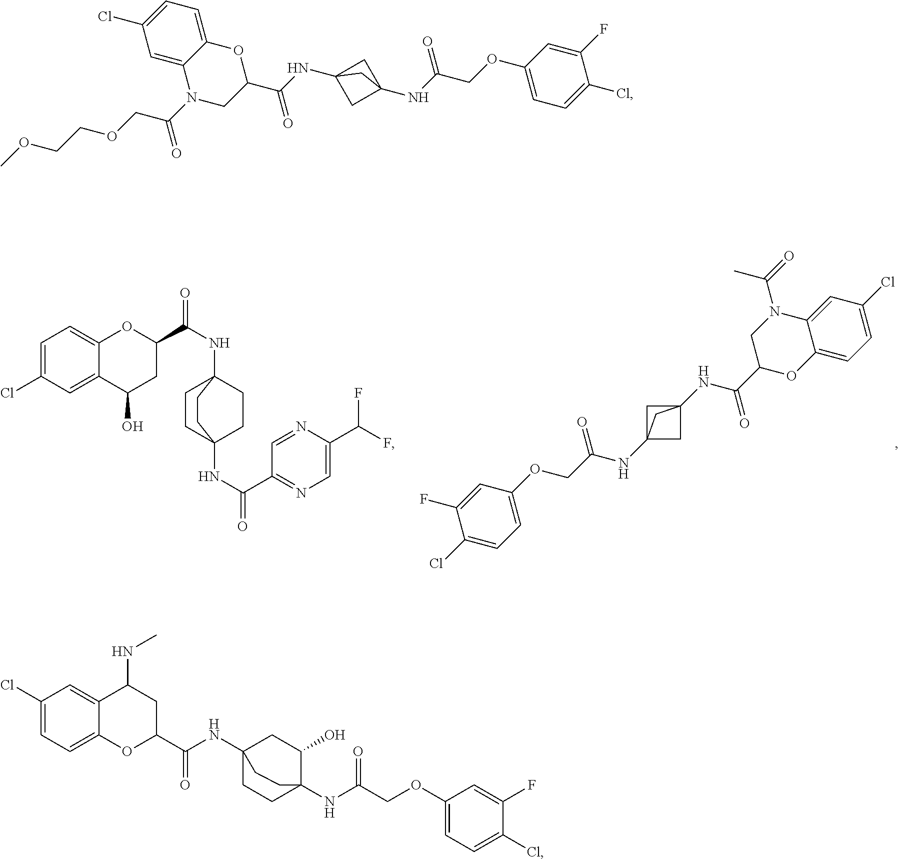 [SMILES: COCCOCC(=O)N1CC(C(=O)NC23CC(NC(=O)COc4ccc(Cl)c(F)c4)(C2)C3)Oc2ccc(Cl)cc21 ; O=C(NC12CCC(NC(=O)[C@H]3C[C@@H](O)c4cc(Cl)ccc4O3)(CC1)CC2)c1cnc(C(F)F)cn1 ; CC(=O)N1CC(C(=O)NC23CC(NC(=O)COc4ccc(Cl)c(F)c4)(C2)C3)Oc2ccc(Cl)cc21 ; CNC1CC(C(=O)NC23CCC(NC(=O)COc4ccc(Cl)c(F)c4)(CC2)[C@@H](O)C3)Oc2ccc(Cl)cc21] 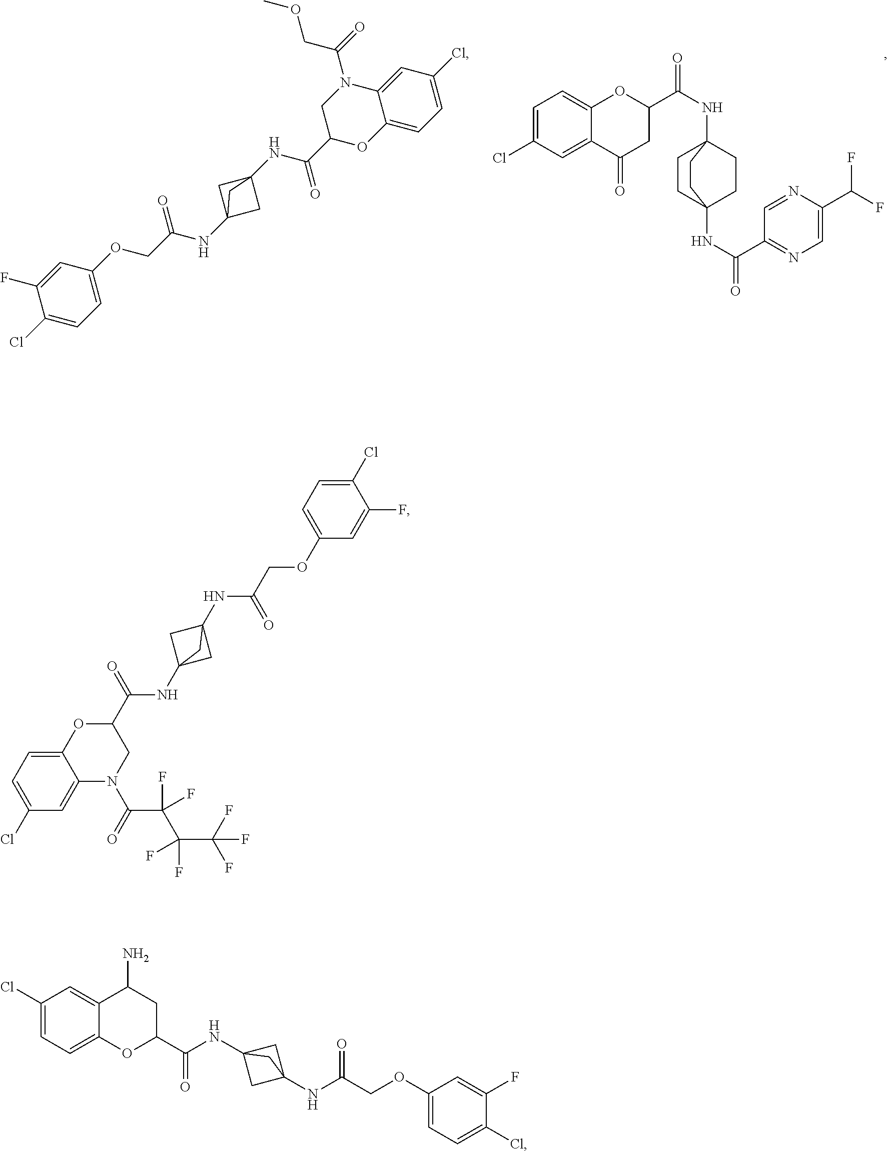 [SMILES: COCC(=O)N1CC(C(=O)NC23CC(NC(=O)COc4ccc(Cl)c(F)c4)(C2)C3)Oc2ccc(Cl)cc21 ; O=C(NC12CCC(NC(=O)C3CC(=O)c4cc(Cl)ccc4O3)(CC1)CC2)c1cnc(C(F)F)cn1 ; O=C(COc1ccc(Cl)c(F)c1)NC12CC(NC(=O)C3CN(C(=O)C(F)(F)C(F)(F)C(F)(F)F)c4cc(Cl)ccc4O3)(C1)C2 ; NC1CC(C(=O)NC23CC(NC(=O)COc4ccc(Cl)c(F)c4)(C2)C3)Oc2ccc(Cl)cc21] 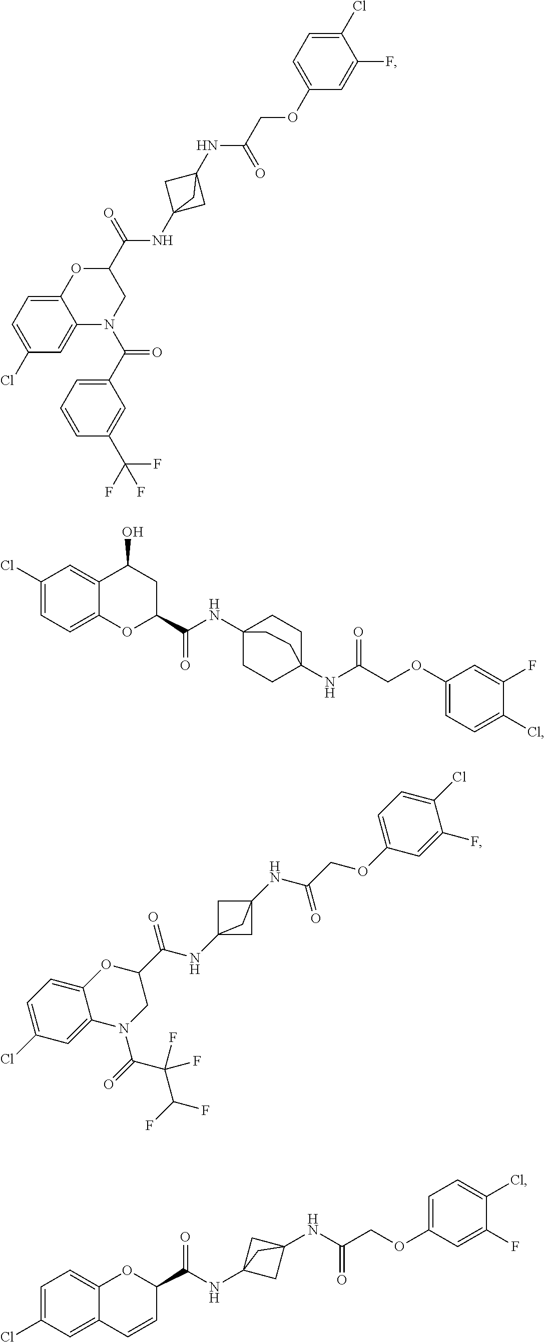 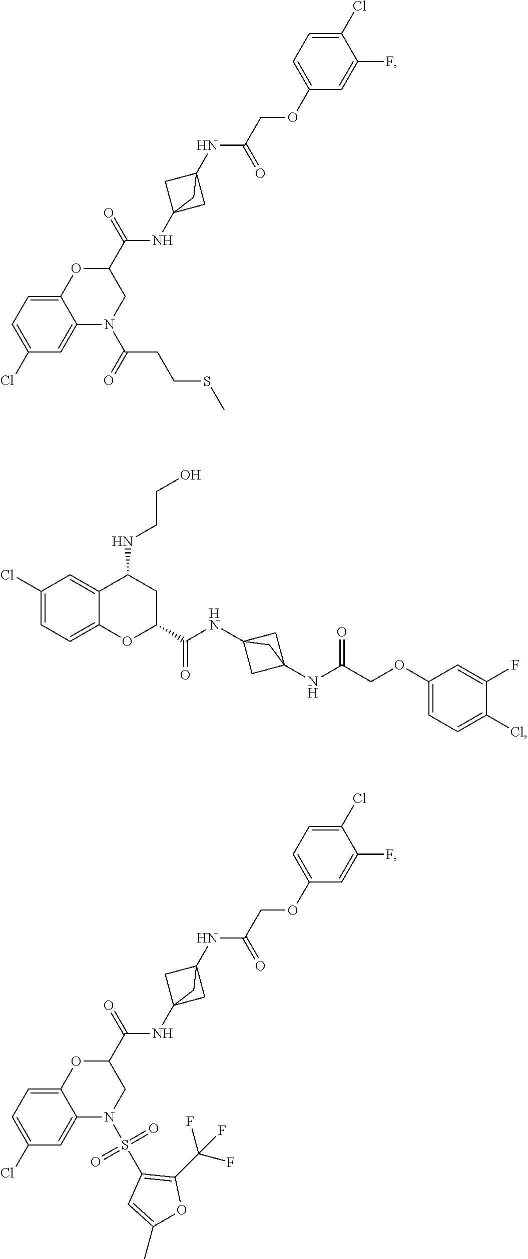 [SMILES: CSCCC(=O)N1CC(C(=O)NC23CC(NC(=O)COc4ccc(Cl)c(F)c4)(C2)C3)Oc2ccc(Cl)cc21 ; O=C(COc1ccc(Cl)c(F)c1)NC12CC(NC(=O)[C@H]3C[C@@H](NCCO)c4cc(Cl)ccc4O3)(C1)C2 ; Cc1cc(S(=O)(=O)N2CC(C(=O)NC34CC(NC(=O)COc5ccc(Cl)c(F)c5)(C3)C4)Oc3ccc(Cl)cc32)c(C(F)(F)F)o1] 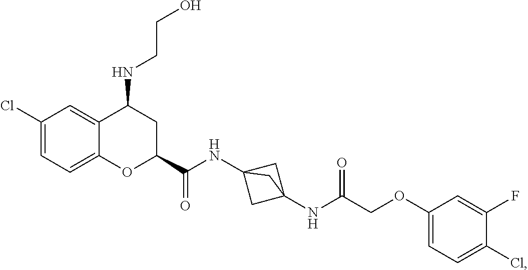 [SMILES: O=C(COc1ccc(Cl)c(F)c1)NC12CC(NC(=O)[C@@H]3C[C@H](NCCO)c4cc(Cl)ccc4O3)(C1)C2] 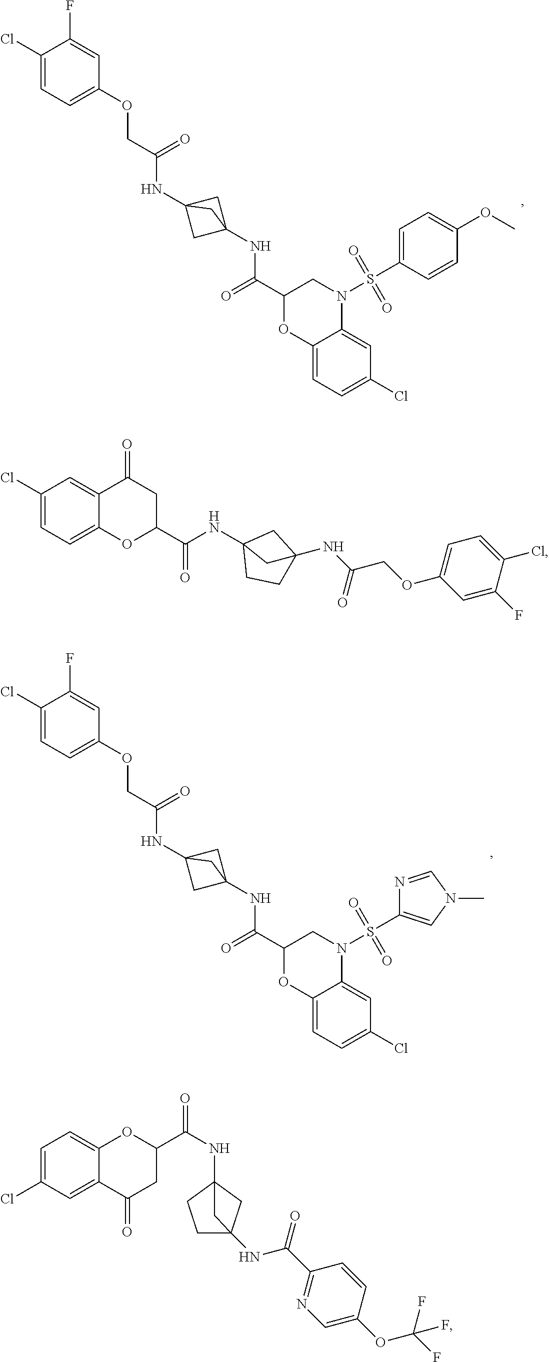 [SMILES: COc1ccc(S(=O)(=O)N2CC(C(=O)NC34CC(NC(=O)COc5ccc(Cl)c(F)c5)(C3)C4)Oc3ccc(Cl)cc32)cc1 ; O=C(COc1ccc(Cl)c(F)c1)NC12CCC(NC(=O)C3CC(=O)c4cc(Cl)ccc4O3)(C1)C2 ; Cn1cnc(S(=O)(=O)N2CC(C(=O)NC34CC(NC(=O)COc5ccc(Cl)c(F)c5)(C3)C4)Oc3ccc(Cl)cc32)c1 ; O=C(NC12CCC(NC(=O)C3CC(=O)c4cc(Cl)ccc4O3)(C1)C2)c1ccc(OC(F)(F)F)cn1] 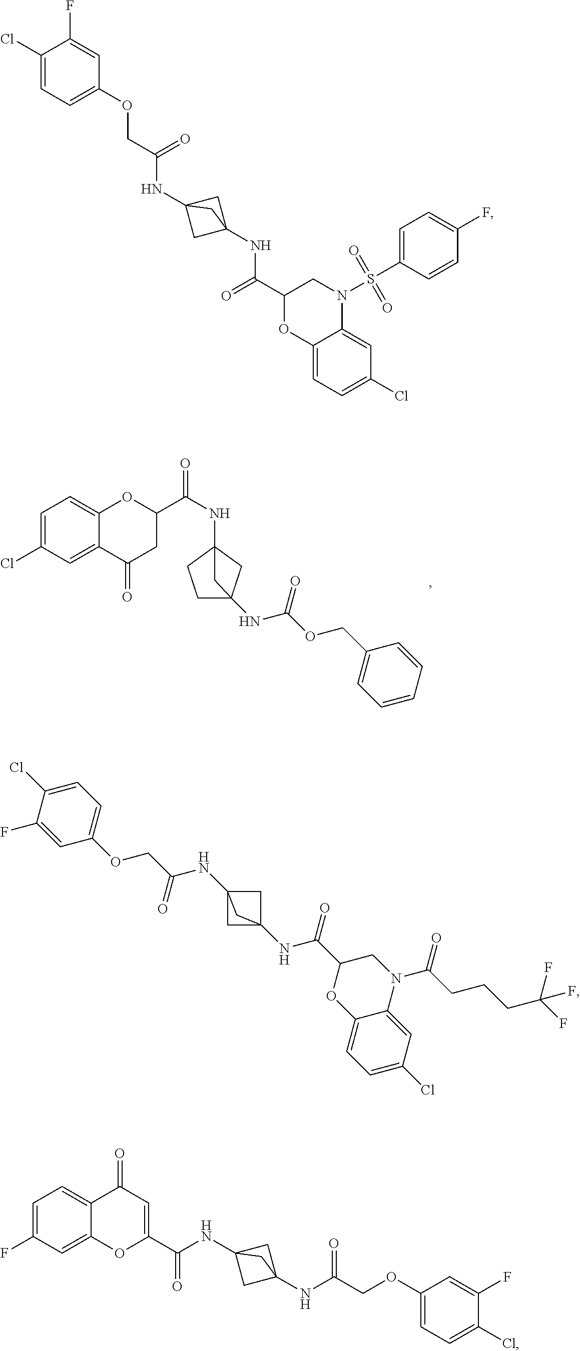 [SMILES: O=C(COc1ccc(Cl)c(F)c1)NC12CC(NC(=O)C3CN(S(=O)(=O)c4ccc(F)cc4)c4cc(Cl)ccc4O3)(C1)C2 ; O=C(NC12CCC(NC(=O)C3CC(=O)c4cc(Cl)ccc4O3)(C1)C2)OCc1ccccc1 ; O=C(COc1ccc(Cl)c(F)c1)NC12CC(NC(=O)C3CN(C(=O)CCCC(F)(F)F)c4cc(Cl)ccc4O3)(C1)C2 ; O=C(COc1ccc(Cl)c(F)c1)NC12CC(NC(=O)c3cc(=O)c4ccc(F)cc4o3)(C1)C2] 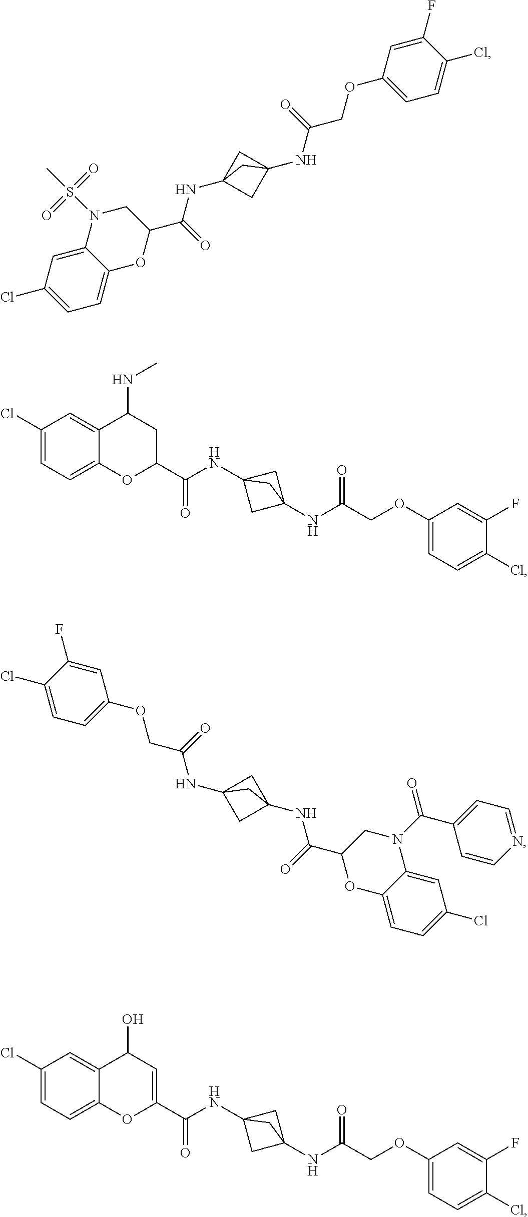 [SMILES: CS(=O)(=O)N1CC(C(=O)NC23CC(NC(=O)COc4ccc(Cl)c(F)c4)(C2)C3)Oc2ccc(Cl)cc21 ; CNC1CC(C(=O)NC23CC(NC(=O)COc4ccc(Cl)c(F)c4)(C2)C3)Oc2ccc(Cl)cc21 ; O=C(COc1ccc(Cl)c(F)c1)NC12CC(NC(=O)C3CN(C(=O)c4ccncc4)c4cc(Cl)ccc4O3)(C1)C2 ; O=C(COc1ccc(Cl)c(F)c1)NC12CC(NC(=O)C3=CC(O)c4cc(Cl)ccc4O3)(C1)C2] 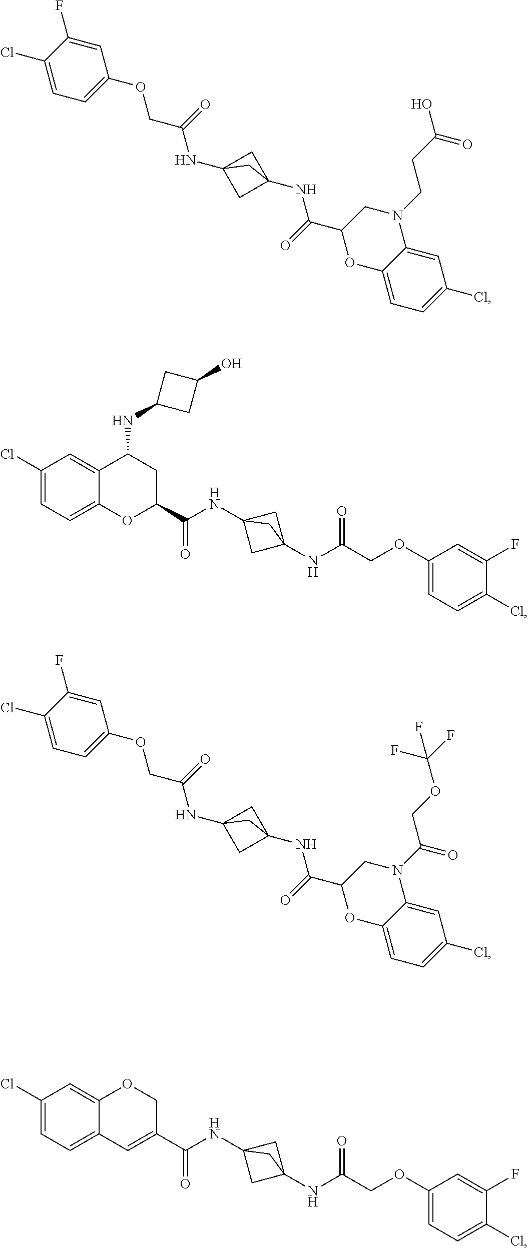 [SMILES: O=C(O)CCN1CC(C(=O)NC23CC(NC(=O)COc4ccc(Cl)c(F)c4)(C2)C3)Oc2ccc(Cl)cc21 ; O=C(COc1ccc(Cl)c(F)c1)NC12CC(NC(=O)[C@@H]3C[C@@H](N[C@H]4C[C@@H](O)C4)c4cc(Cl)ccc4O3)(C1)C2 ; O=C(COc1ccc(Cl)c(F)c1)NC12CC(NC(=O)C3CN(C(=O)COC(F)(F)F)c4cc(Cl)ccc4O3)(C1)C2 ; O=C(COc1ccc(Cl)c(F)c1)NC12CC(NC(=O)C3=Cc4ccc(Cl)cc4OC3)(C1)C2] 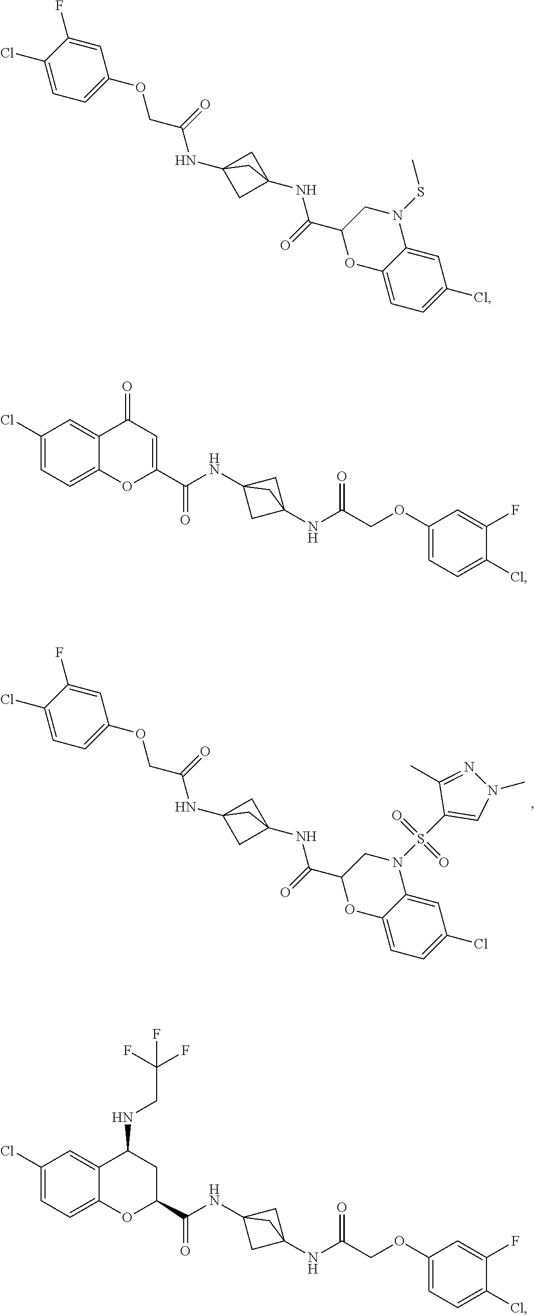 [SMILES: CSN1CC(C(=O)NC23CC(NC(=O)COc4ccc(Cl)c(F)c4)(C2)C3)Oc2ccc(Cl)cc21 ; O=C(COc1ccc(Cl)c(F)c1)NC12CC(NC(=O)c3cc(=O)c4cc(Cl)ccc4o3)(C1)C2 ; Cc1nn(C)cc1S(=O)(=O)N1CC(C(=O)NC23CC(NC(=O)COc4ccc(Cl)c(F)c4)(C2)C3)Oc2ccc(Cl)cc21 ; O=C(COc1ccc(Cl)c(F)c1)NC12CC(NC(=O)[C@@H]3C[C@H](NCC(F)(F)F)c4cc(Cl)ccc4O3)(C1)C2] 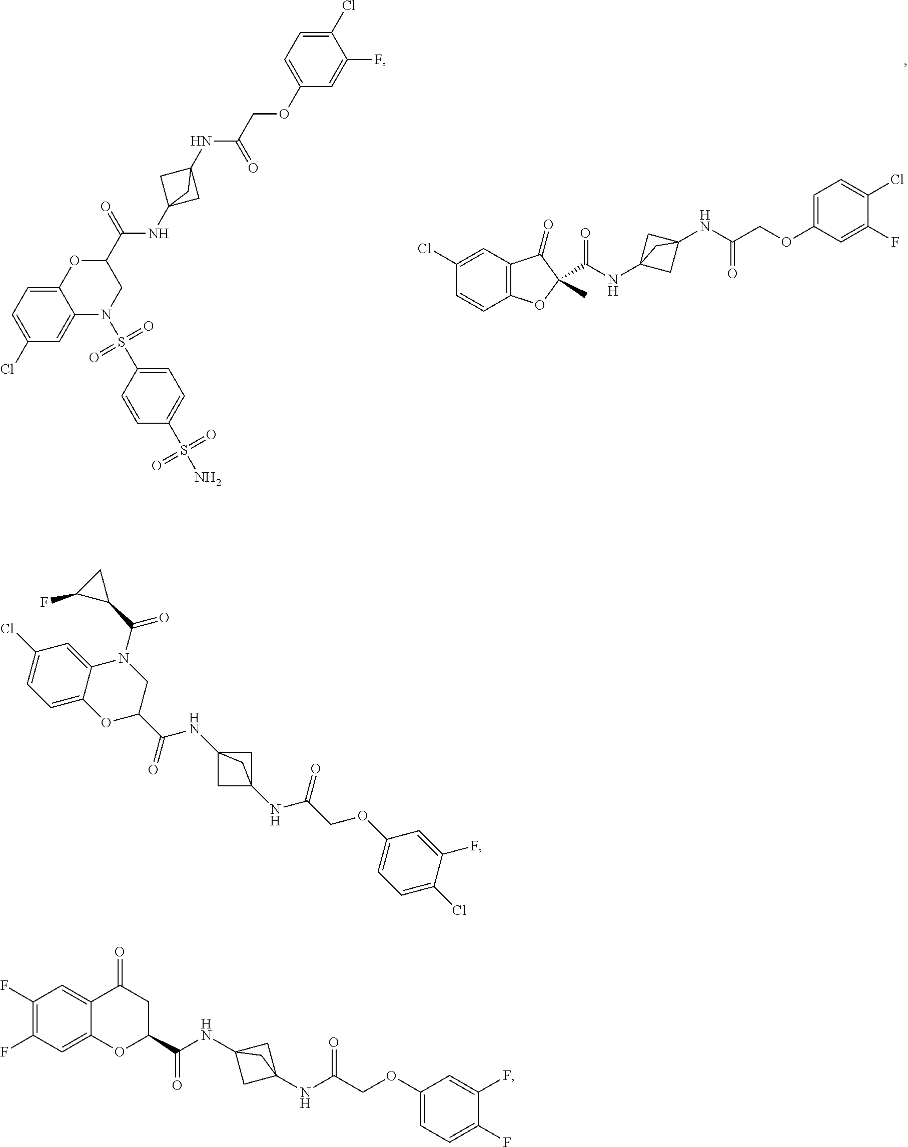 [SMILES: NS(=O)(=O)c1ccc(S(=O)(=O)N2CC(C(=O)NC34CC(NC(=O)COc5ccc(Cl)c(F)c5)(C3)C4)Oc3ccc(Cl)cc32)cc1 ; C[C@@]1(C(=O)NC23CC(NC(=O)COc4ccc(Cl)c(F)c4)(C2)C3)Oc2ccc(Cl)cc2C1=O ; O=C(COc1ccc(Cl)c(F)c1)NC12CC(NC(=O)C3CN(C(=O)[C@@H]4C[C@@H]4F)c4cc(Cl)ccc4O3)(C1)C2 ; O=C(COc1ccc(F)c(F)c1)NC12CC(NC(=O)[C@@H]3CC(=O)c4cc(F)c(F)cc4O3)(C1)C2] 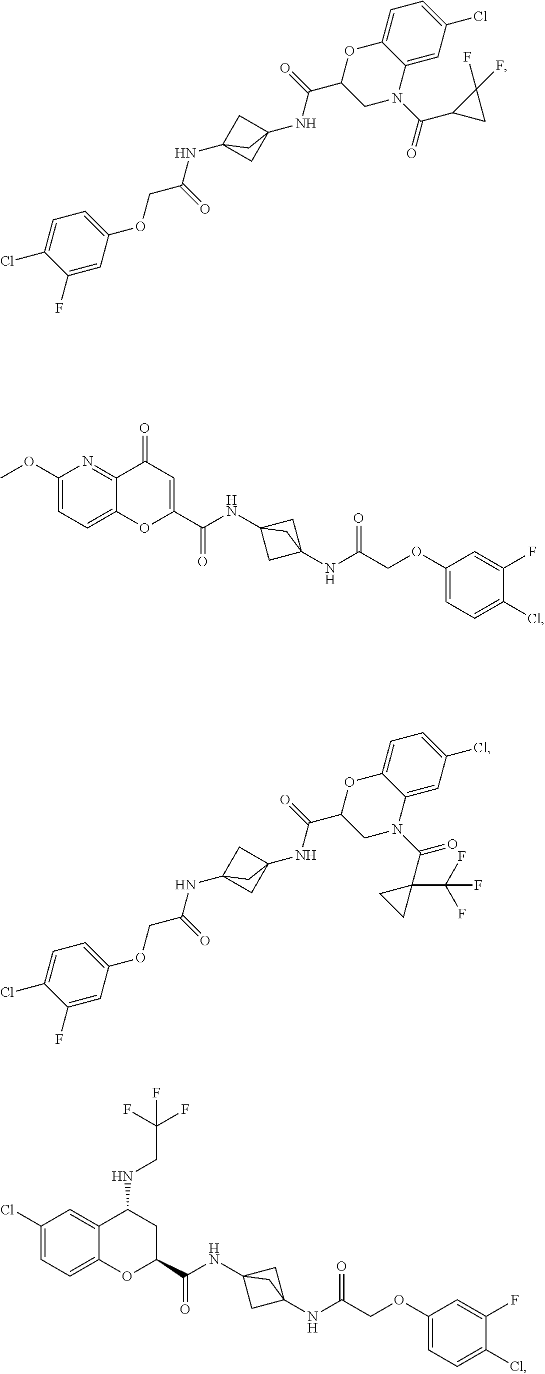 [SMILES: O=C(COc1ccc(Cl)c(F)c1)NC12CC(NC(=O)C3CN(C(=O)C4CC4(F)F)c4cc(Cl)ccc4O3)(C1)C2 ; COc1ccc2oc(C(=O)NC34CC(NC(=O)COc5ccc(Cl)c(F)c5)(C3)C4)cc(=O)c2n1 ; O=C(COc1ccc(Cl)c(F)c1)NC12CC(NC(=O)C3CN(C(=O)C4(C(F)(F)F)CC4)c4cc(Cl)ccc4O3)(C1)C2 ; O=C(COc1ccc(Cl)c(F)c1)NC12CC(NC(=O)[C@@H]3C[C@@H](NCC(F)(F)F)c4cc(Cl)ccc4O3)(C1)C2] 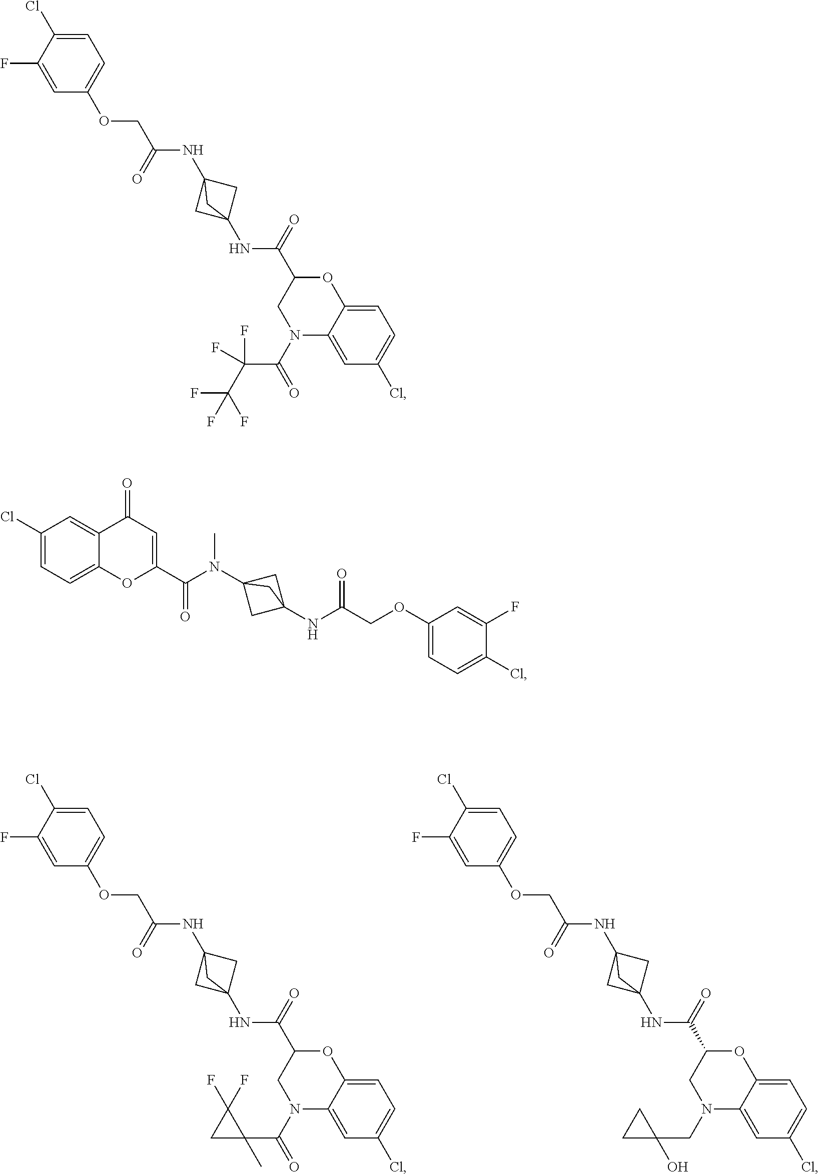 [SMILES: O=C(COc1ccc(Cl)c(F)c1)NC12CC(NC(=O)C3CN(C(=O)C(F)(F)C(F)(F)F)c4cc(Cl)ccc4O3)(C1)C2 ; CN(C(=O)c1cc(=O)c2cc(Cl)ccc2o1)C12CC(NC(=O)COc3ccc(Cl)c(F)c3)(C1)C2 ; CC1(C(=O)N2CC(C(=O)NC34CC(NC(=O)COc5ccc(Cl)c(F)c5)(C3)C4)Oc3ccc(Cl)cc32)CC1(F)F ; O=C(COc1ccc(Cl)c(F)c1)NC12CC(NC(=O)[C@H]3CN(CC4(O)CC4)c4cc(Cl)ccc4O3)(C1)C2] 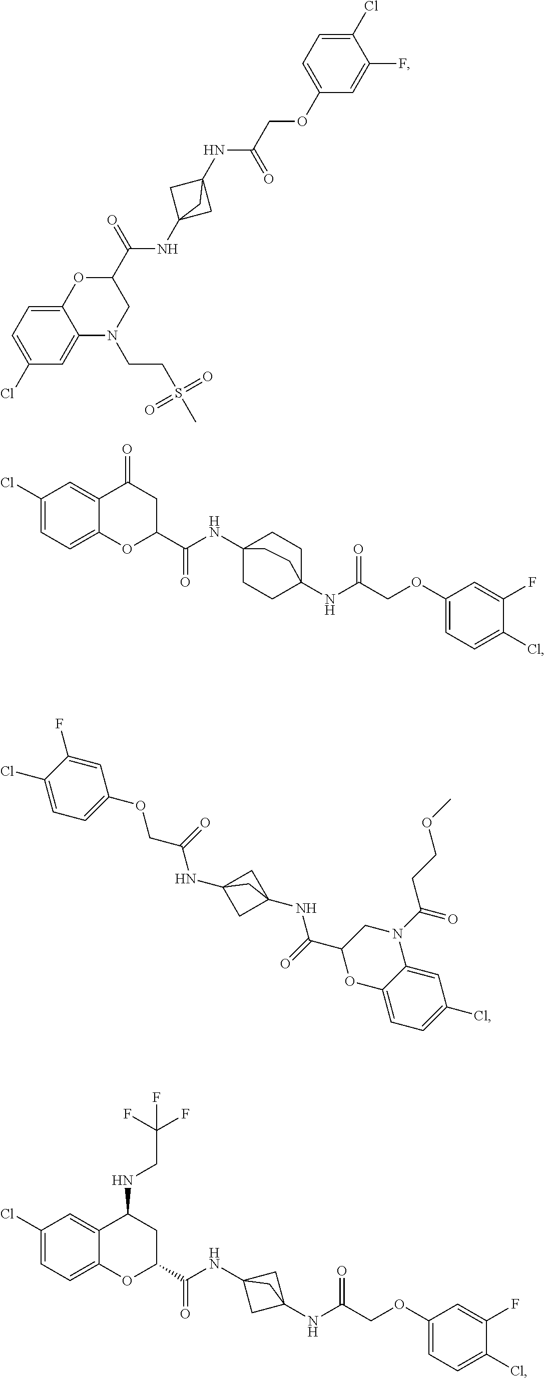 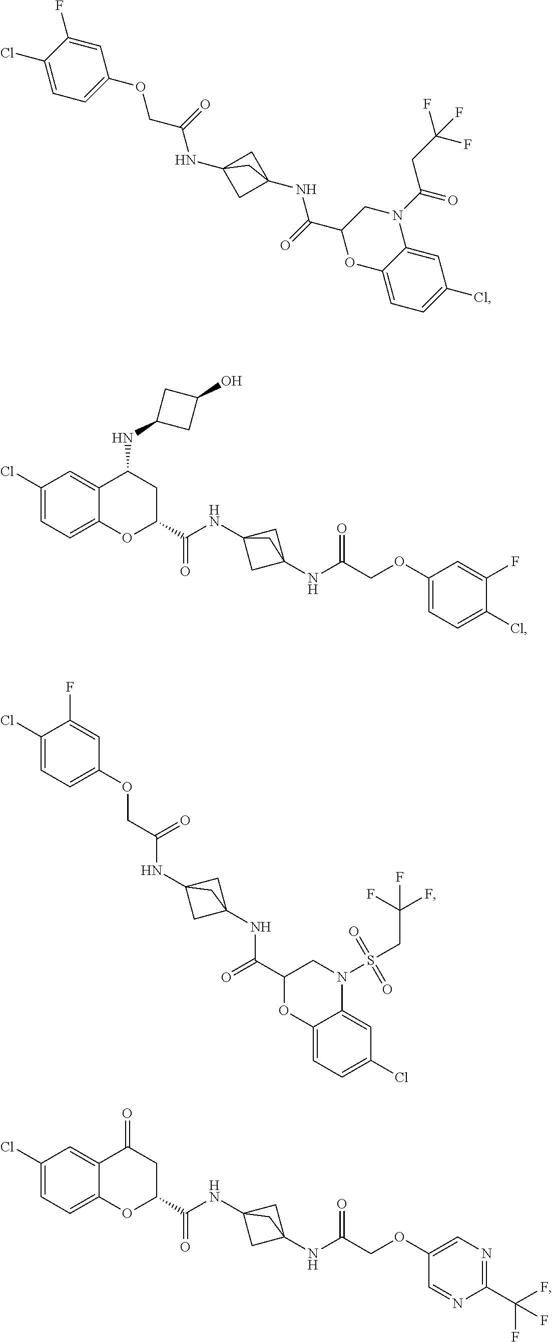 [SMILES: O=C(COc1ccc(Cl)c(F)c1)NC12CC(NC(=O)C3CN(C(=O)CC(F)(F)F)c4cc(Cl)ccc4O3)(C1)C2 ; O=C(COc1ccc(Cl)c(F)c1)NC12CC(NC(=O)[C@H]3C[C@@H](N[C@H]4C[C@@H](O)C4)c4cc(Cl)ccc4O3)(C1)C2 ; O=C(COc1ccc(Cl)c(F)c1)NC12CC(NC(=O)C3CN(S(=O)(=O)CC(F)(F)F)c4cc(Cl)ccc4O3)(C1)C2 ; O=C(COc1cnc(C(F)(F)F)nc1)NC12CC(NC(=O)[C@H]3CC(=O)c4cc(Cl)ccc4O3)(C1)C2] 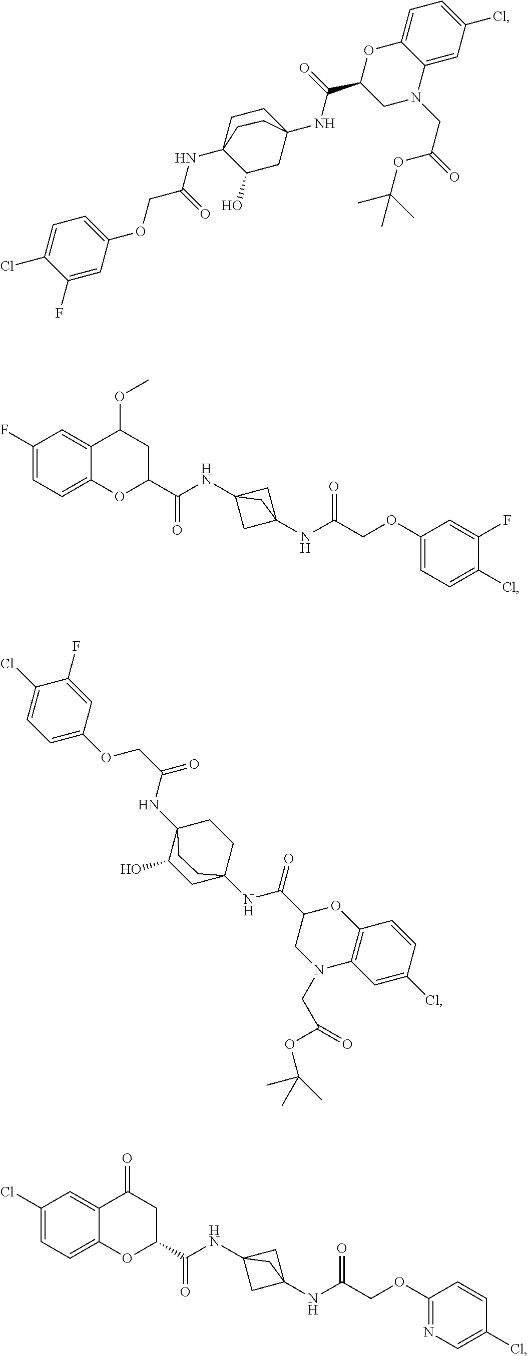 [SMILES: CC(C)(C)OC(=O)CN1C[C@@H](C(=O)NC23CCC(NC(=O)COc4ccc(Cl)c(F)c4)(CC2)[C@@H](O)C3)Oc2ccc(Cl)cc21 ; COC1CC(C(=O)NC23CC(NC(=O)COc4ccc(Cl)c(F)c4)(C2)C3)Oc2ccc(F)cc21 ; CC(C)(C)OC(=O)CN1CC(C(=O)NC23CCC(NC(=O)COc4ccc(Cl)c(F)c4)(CC2)[C@@H](O)C3)Oc2ccc(Cl)cc21 ; O=C(COc1ccc(Cl)cn1)NC12CC(NC(=O)[C@H]3CC(=O)c4cc(Cl)ccc4O3)(C1)C2] 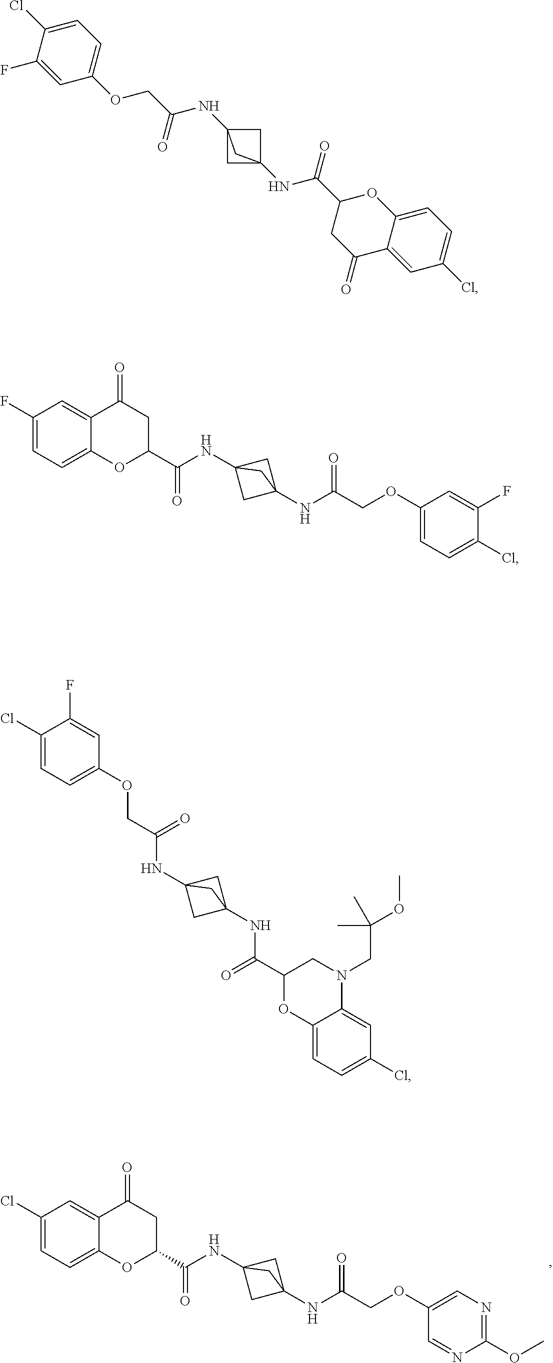 [SMILES: O=C(COc1ccc(Cl)c(F)c1)NC12CC(NC(=O)C3CC(=O)c4cc(Cl)ccc4O3)(C1)C2 ; O=C(COc1ccc(Cl)c(F)c1)NC12CC(NC(=O)C3CC(=O)c4cc(F)ccc4O3)(C1)C2 ; COC(C)(C)CN1CC(C(=O)NC23CC(NC(=O)COc4ccc(Cl)c(F)c4)(C2)C3)Oc2ccc(Cl)cc21 ; COc1ncc(OCC(=O)NC23CC(NC(=O)[C@H]4CC(=O)c5cc(Cl)ccc5O4)(C2)C3)cn1] 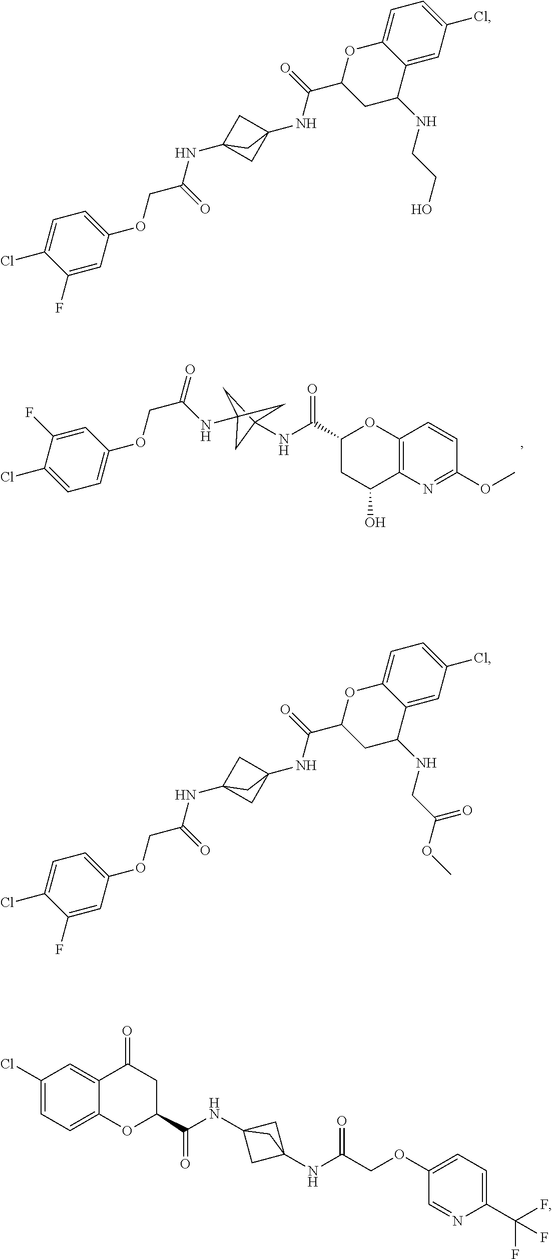 [SMILES: O=C(COc1ccc(Cl)c(F)c1)NC12CC(NC(=O)C3CC(NCCO)c4cc(Cl)ccc4O3)(C1)C2 ; COc1ccc2c(n1)[C@H](O)C[C@H](C(=O)NC13CC(NC(=O)COc4ccc(Cl)c(F)c4)(C1)C3)O2 ; COC(=O)CNC1CC(C(=O)NC23CC(NC(=O)COc4ccc(Cl)c(F)c4)(C2)C3)Oc2ccc(Cl)cc21 ; O=C(COc1ccc(C(F)(F)F)nc1)NC12CC(NC(=O)[C@@H]3CC(=O)c4cc(Cl)ccc4O3)(C1)C2] 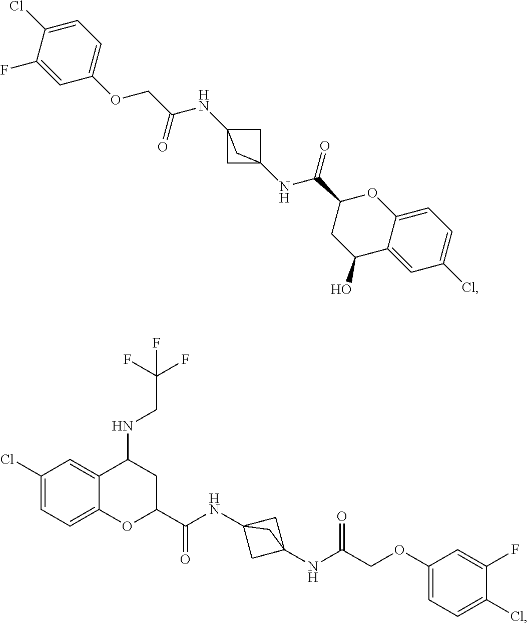 [SMILES: O=C(COc1ccc(Cl)c(F)c1)NC12CC(NC(=O)[C@@H]3C[C@H](O)c4cc(Cl)ccc4O3)(C1)C2 ; O=C(COc1ccc(Cl)c(F)c1)NC12CC(NC(=O)C3CC(NCC(F)(F)F)c4cc(Cl)ccc4O3)(C1)C2] 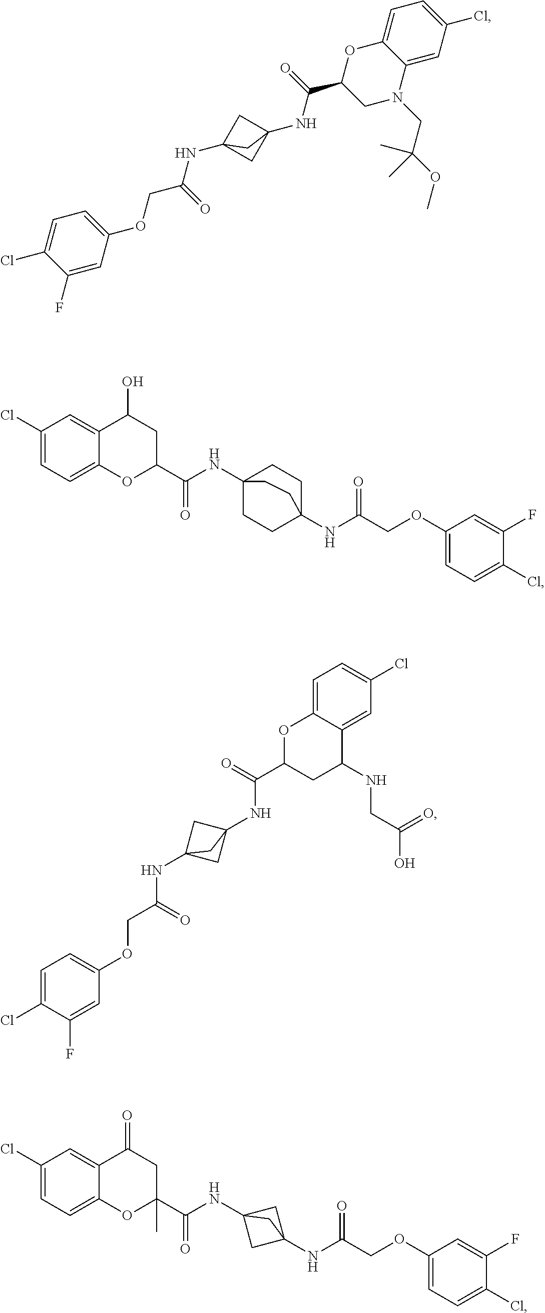 [SMILES: COC(C)(C)CN1C[C@@H](C(=O)NC23CC(NC(=O)COc4ccc(Cl)c(F)c4)(C2)C3)Oc2ccc(Cl)cc21 ; O=C(COc1ccc(Cl)c(F)c1)NC12CCC(NC(=O)C3CC(O)c4cc(Cl)ccc4O3)(CC1)CC2 ; O=C(O)CNC1CC(C(=O)NC23CC(NC(=O)COc4ccc(Cl)c(F)c4)(C2)C3)Oc2ccc(Cl)cc21 ; CC1(C(=O)NC23CC(NC(=O)COc4ccc(Cl)c(F)c4)(C2)C3)CC(=O)c2cc(Cl)ccc2O1] 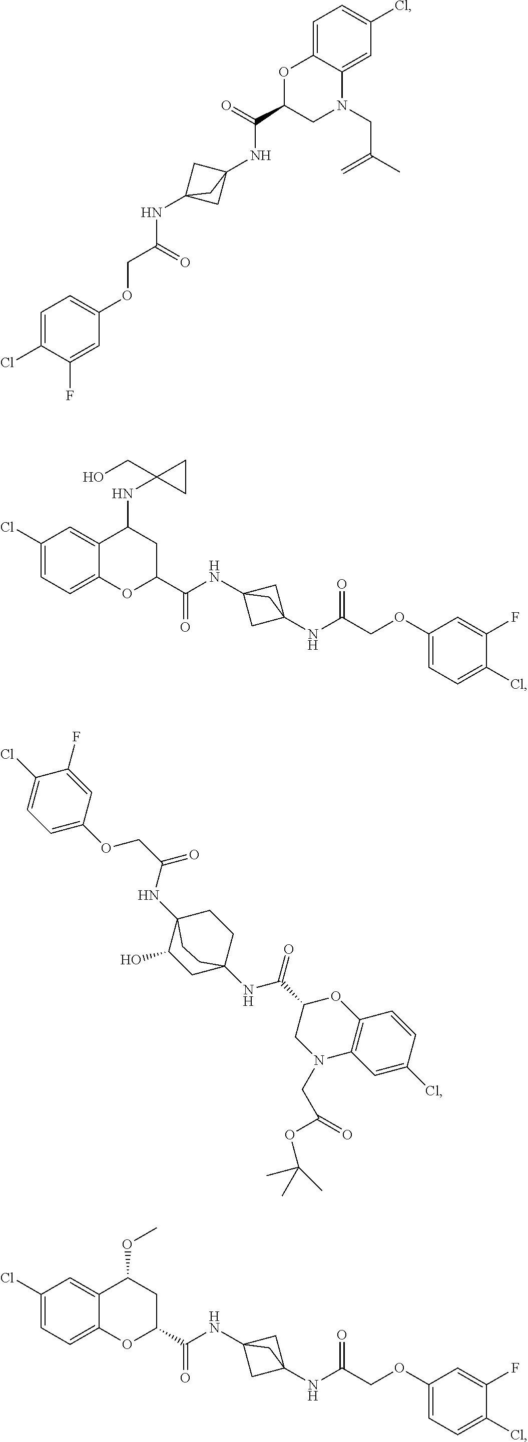 [SMILES: C=C(C)CN1C[C@@H](C(=O)NC23CC(NC(=O)COc4ccc(Cl)c(F)c4)(C2)C3)Oc2ccc(Cl)cc21 ; O=C(COc1ccc(Cl)c(F)c1)NC12CC(NC(=O)C3CC(NC4(CO)CC4)c4cc(Cl)ccc4O3)(C1)C2 ; CC(C)(C)OC(=O)CN1C[C@H](C(=O)NC23CCC(NC(=O)COc4ccc(Cl)c(F)c4)(CC2)[C@@H](O)C3)Oc2ccc(Cl)cc21 ; CO[C@@H]1C[C@H](C(=O)NC23CC(NC(=O)COc4ccc(Cl)c(F)c4)(C2)C3)Oc2ccc(Cl)cc21] 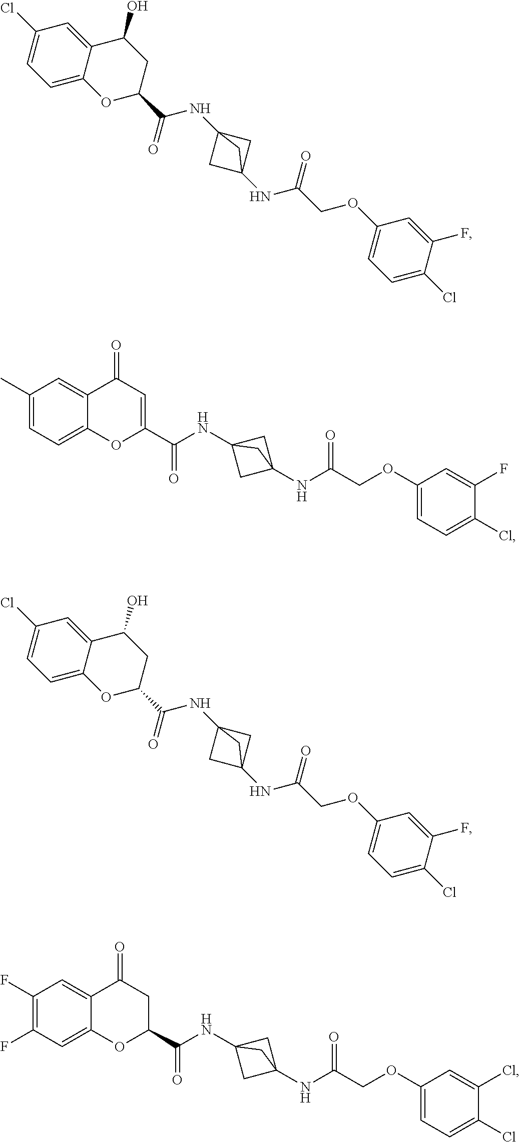 [SMILES: O=C(COc1ccc(Cl)c(F)c1)NC12CC(NC(=O)[C@@H]3C[C@H](O)c4cc(Cl)ccc4O3)(C1)C2 ; Cc1ccc2oc(C(=O)NC34CC(NC(=O)COc5ccc(Cl)c(F)c5)(C3)C4)cc(=O)c2c1 ; O=C(COc1ccc(Cl)c(F)c1)NC12CC(NC(=O)[C@H]3C[C@@H](O)c4cc(Cl)ccc4O3)(C1)C2 ; O=C(COc1ccc(Cl)c(Cl)c1)NC12CC(NC(=O)[C@@H]3CC(=O)c4cc(F)c(F)cc4O3)(C1)C2] 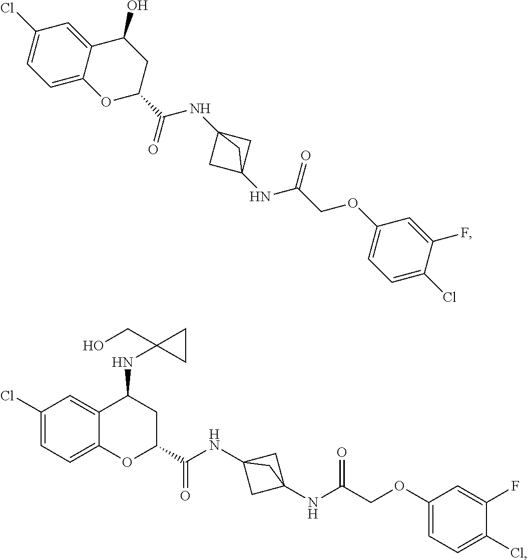 [SMILES: O=C(COc1ccc(Cl)c(F)c1)NC12CC(NC(=O)[C@H]3C[C@H](O)c4cc(Cl)ccc4O3)(C1)C2 ; O=C(COc1ccc(Cl)c(F)c1)NC12CC(NC(=O)[C@H]3C[C@H](NC4(CO)CC4)c4cc(Cl)ccc4O3)(C1)C2] 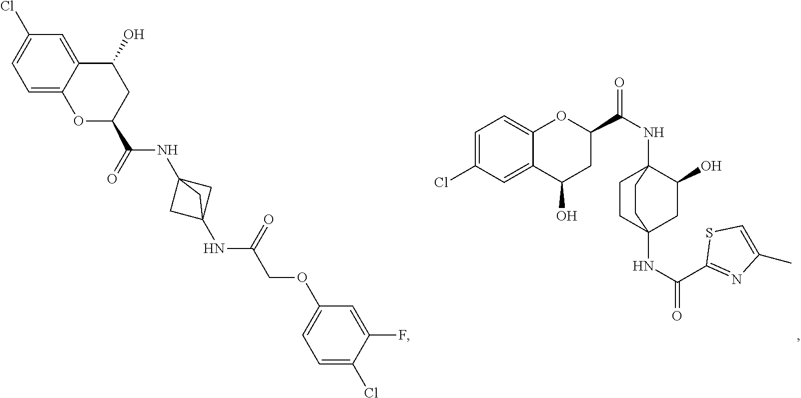 [SMILES: O=C(COc1ccc(Cl)c(F)c1)NC12CC(NC(=O)[C@@H]3C[C@@H](O)c4cc(Cl)ccc4O3)(C1)C2 ; Cc1csc(C(=O)NC23CCC(NC(=O)[C@H]4C[C@@H](O)c5cc(Cl)ccc5O4)(CC2)[C@@H](O)C3)n1] 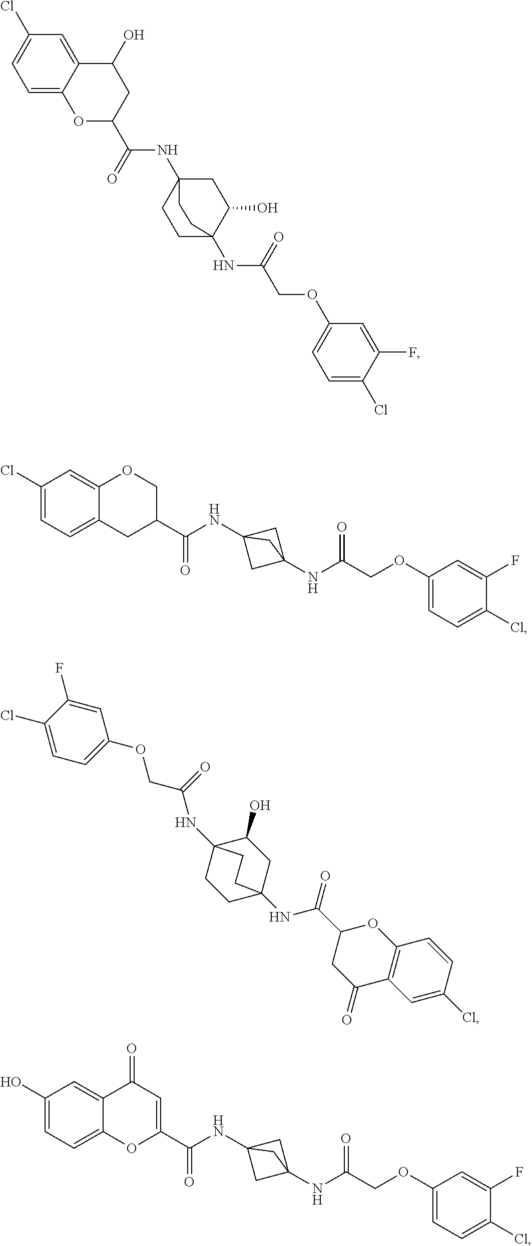 [SMILES: O=C(COc1ccc(Cl)c(F)c1)NC12CCC(NC(=O)C3CC(O)c4cc(Cl)ccc4O3)(CC1)C[C@@H]2O ; O=C(COc1ccc(Cl)c(F)c1)NC12CC(NC(=O)C3COc4cc(Cl)ccc4C3)(C1)C2 ; O=C(COc1ccc(Cl)c(F)c1)NC12CCC(NC(=O)C3CC(=O)c4cc(Cl)ccc4O3)(CC1)C[C@@H]2O ; O=C(COc1ccc(Cl)c(F)c1)NC12CC(NC(=O)c3cc(=O)c4cc(O)ccc4o3)(C1)C2] 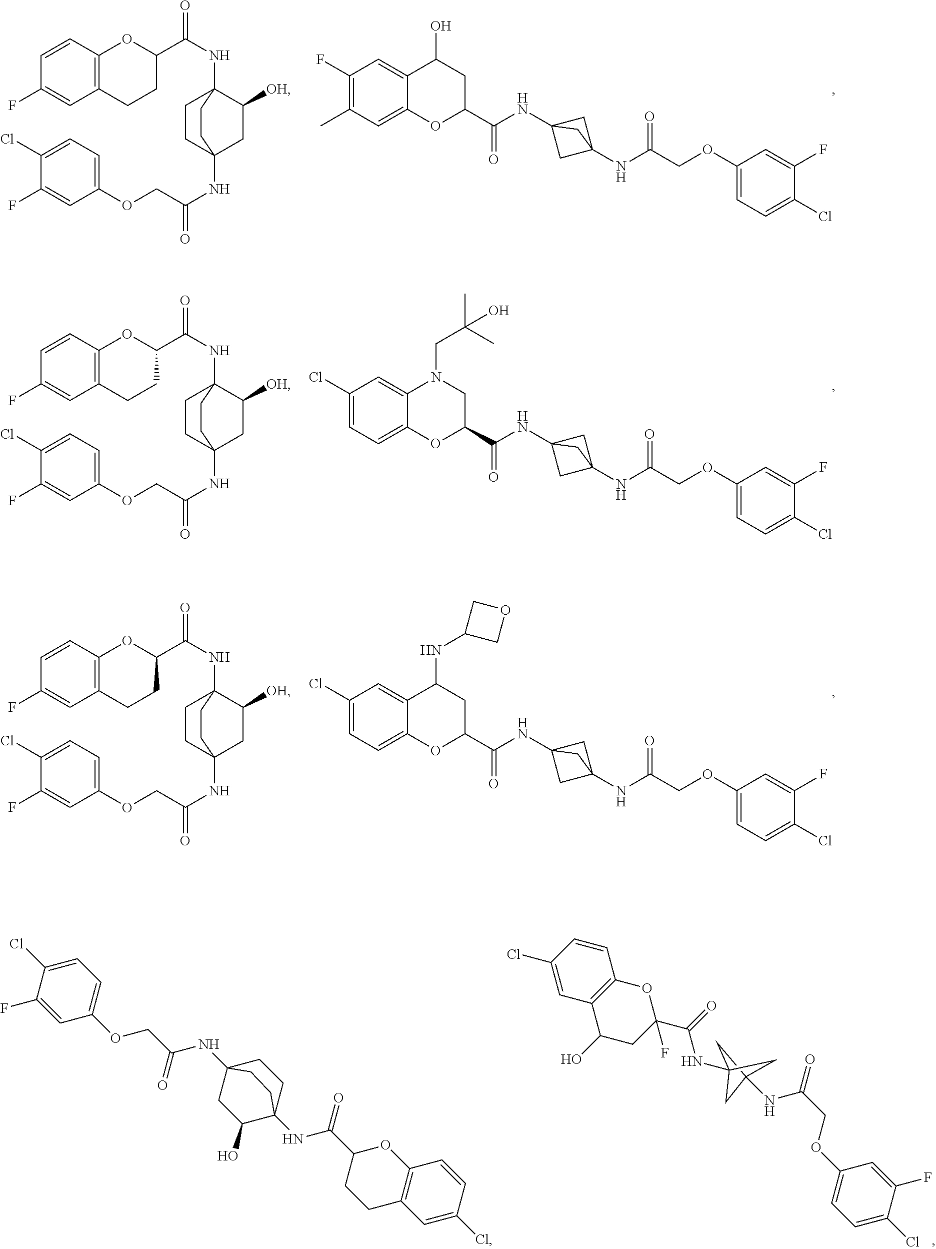 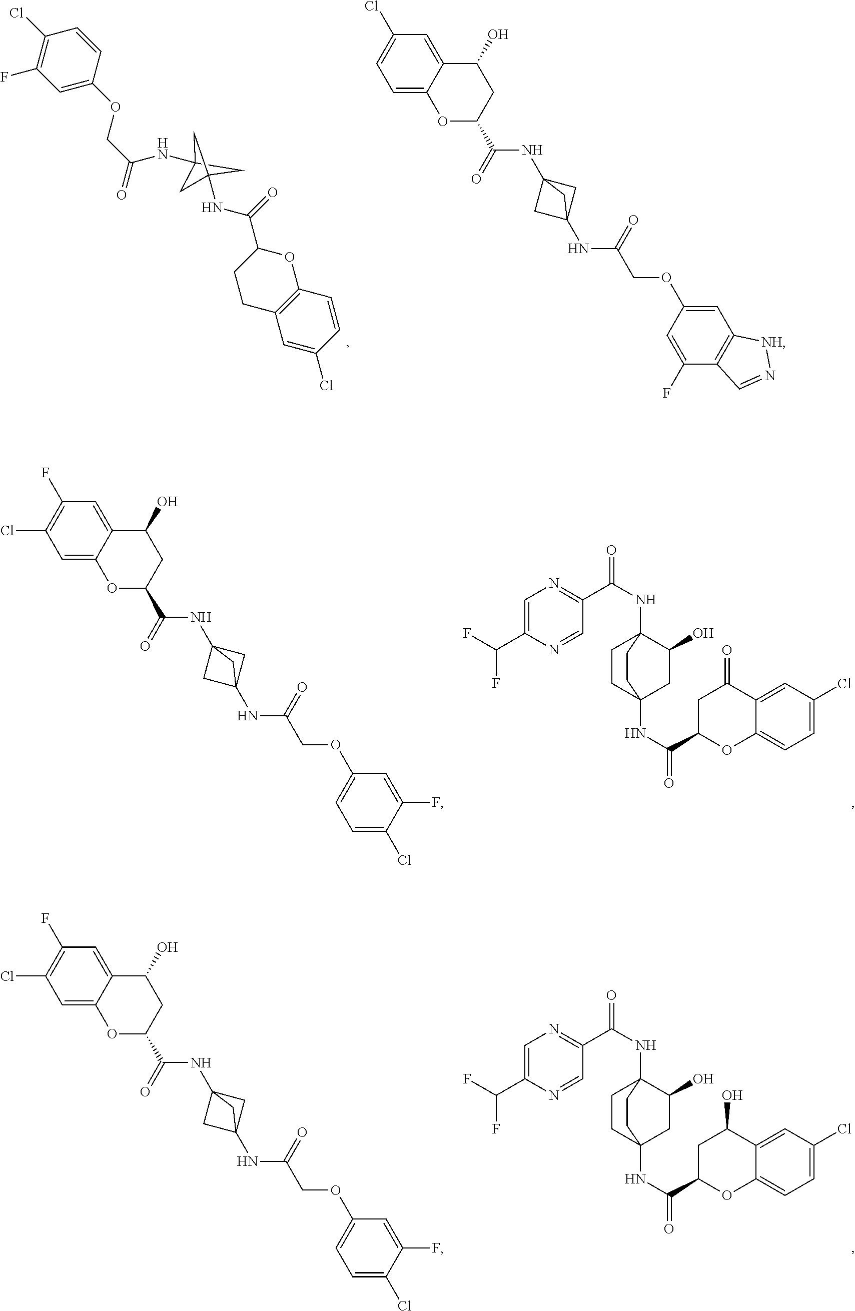 [SMILES: O=C(COc1ccc(Cl)c(F)c1)NC12CC(NC(=O)C3CCc4cc(Cl)ccc4O3)(C1)C2 ; O=C(COc1cc(F)c2cn[nH]c2c1)NC12CC(NC(=O)[C@H]3C[C@@H](O)c4cc(Cl)ccc4O3)(C1)C2 ; O=C(COc1ccc(Cl)c(F)c1)NC12CC(NC(=O)[C@@H]3C[C@H](O)c4cc(F)c(Cl)cc4O3)(C1)C2 ; O=C(NC12CCC(NC(=O)[C@H]3CC(=O)c4cc(Cl)ccc4O3)(CC1)C[C@@H]2O)c1cnc(C(F)F)cn1 ; O=C(COc1ccc(Cl)c(F)c1)NC12CC(NC(=O)[C@H]3C[C@@H](O)c4cc(F)c(Cl)cc4O3)(C1)C2 ; O=C(NC12CCC(NC(=O)[C@H]3C[C@@H](O)c4cc(Cl)ccc4O3)(CC1)C[C@@H]2O)c1cnc(C(F)F)cn1] 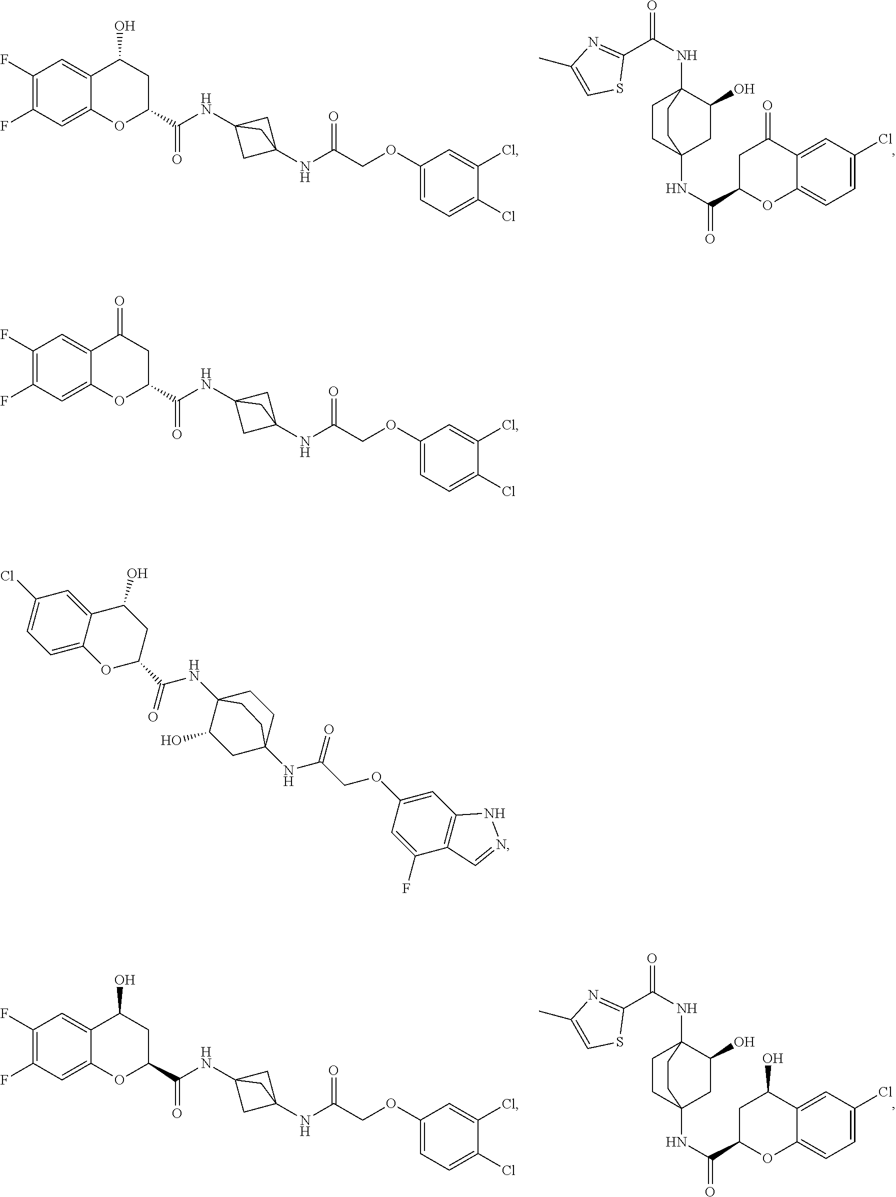 [SMILES: O=C(COc1ccc(Cl)c(Cl)c1)NC12CC(NC(=O)[C@H]3C[C@@H](O)c4cc(F)c(F)cc4O3)(C1)C2 ; Cc1csc(C(=O)NC23CCC(NC(=O)[C@H]4CC(=O)c5cc(Cl)ccc5O4)(CC2)C[C@@H]3O)n1 ; O=C(COc1ccc(Cl)c(Cl)c1)NC12CC(NC(=O)[C@H]3CC(=O)c4cc(F)c(F)cc4O3)(C1)C2 ; O=C(COc1cc(F)c2cn[nH]c2c1)NC12CCC(NC(=O)[C@H]3C[C@@H](O)c4cc(Cl)ccc4O3)(CC1)[C@@H](O)C2 ; O=C(COc1ccc(Cl)c(Cl)c1)NC12CC(NC(=O)[C@@H]3C[C@H](O)c4cc(F)c(F)cc4O3)(C1)C2 ; Cc1csc(C(=O)NC23CCC(NC(=O)[C@H]4C[C@@H](O)c5cc(Cl)ccc5O4)(CC2)C[C@@H]3O)n1] 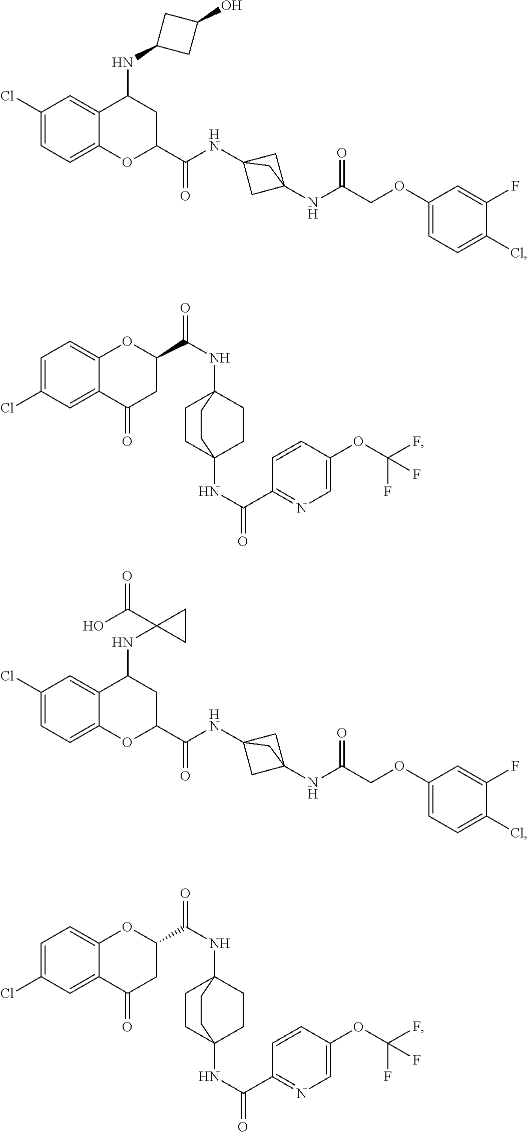 [SMILES: O=C(COc1ccc(Cl)c(F)c1)NC12CC(NC(=O)C3CC(N[C@H]4C[C@@H](O)C4)c4cc(Cl)ccc4O3)(C1)C2 ; O=C(NC12CCC(NC(=O)[C@H]3CC(=O)c4cc(Cl)ccc4O3)(CC1)CC2)c1ccc(OC(F)(F)F)cn1 ; O=C(COc1ccc(Cl)c(F)c1)NC12CC(NC(=O)C3CC(NC4(C(=O)O)CC4)c4cc(Cl)ccc4O3)(C1)C2 ; O=C(NC12CCC(NC(=O)[C@@H]3CC(=O)c4cc(Cl)ccc4O3)(CC1)CC2)c1ccc(OC(F)(F)F)cn1] 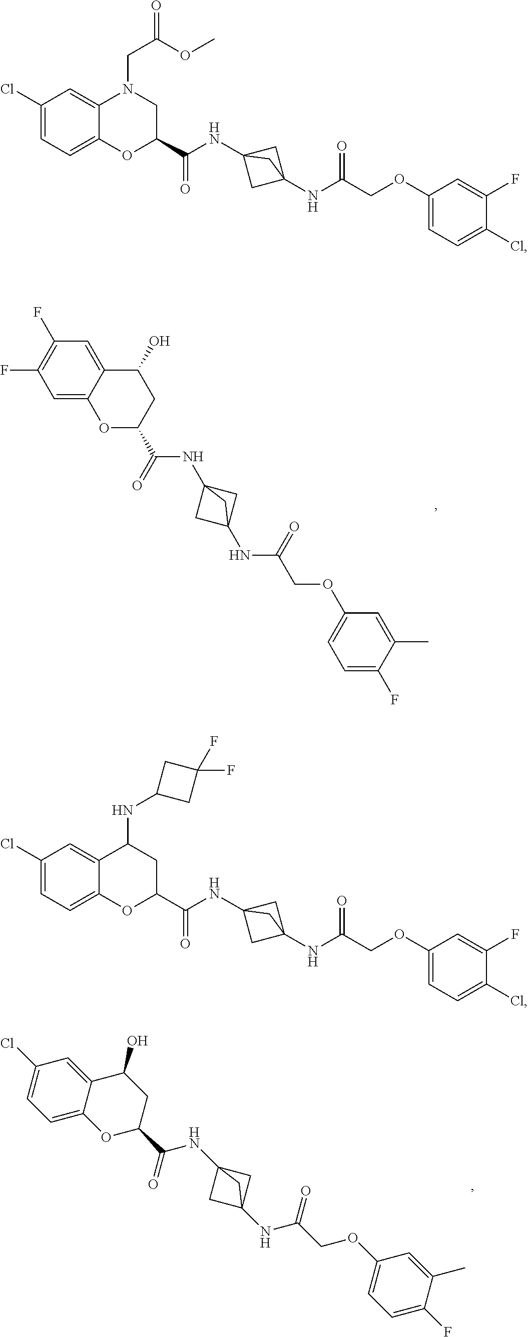 [SMILES: COC(=O)CN1C[C@@H](C(=O)NC23CC(NC(=O)COc4ccc(Cl)c(F)c4)(C2)C3)Oc2ccc(Cl)cc21 ; Cc1cc(OCC(=O)NC23CC(NC(=O)[C@H]4C[C@@H](O)c5cc(F)c(F)cc5O4)(C2)C3)ccc1F ; O=C(COc1ccc(Cl)c(F)c1)NC12CC(NC(=O)C3CC(NC4CC(F)(F)C4)c4cc(Cl)ccc4O3)(C1)C2 ; Cc1cc(OCC(=O)NC23CC(NC(=O)[C@@H]4C[C@H](O)c5cc(Cl)ccc5O4)(C2)C3)ccc1F] 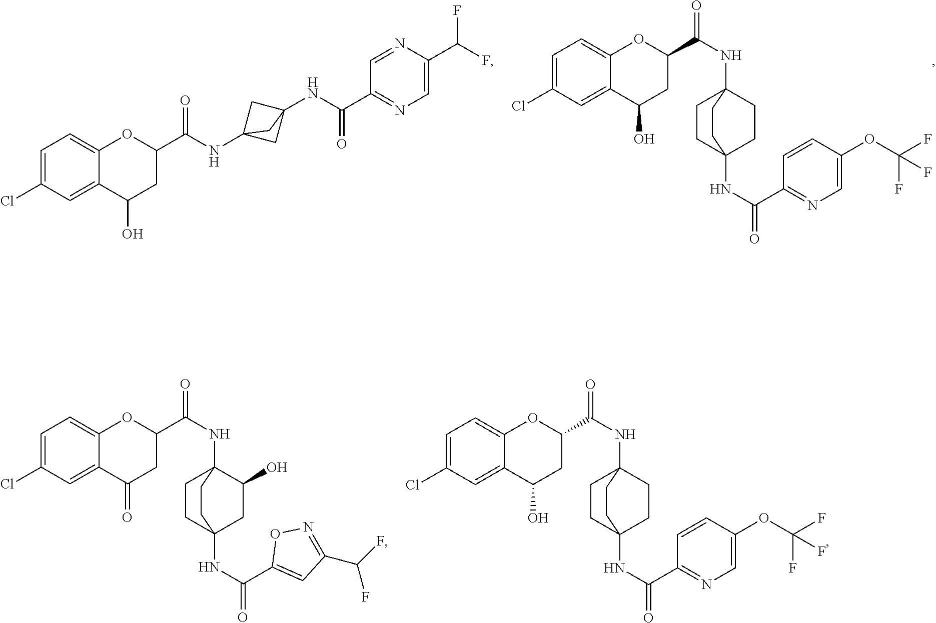 [SMILES: O=C(NC12CC(NC(=O)C3CC(O)c4cc(Cl)ccc4O3)(C1)C2)c1cnc(C(F)F)cn1 ; O=C(NC12CCC(NC(=O)[C@H]3C[C@@H](O)c4cc(Cl)ccc4O3)(CC1)CC2)c1ccc(OC(F)(F)F)cn1 ; O=C(NC12CCC(NC(=O)C3CC(=O)c4cc(Cl)ccc4O3)(CC1)[C@@H](O)C2)c1cc(C(F)F)no1 ; O=C(NC12CCC(NC(=O)[C@@H]3C[C@H](O)c4cc(Cl)ccc4O3)(CC1)CC2)c1ccc(OC(F)(F)F)cn1] 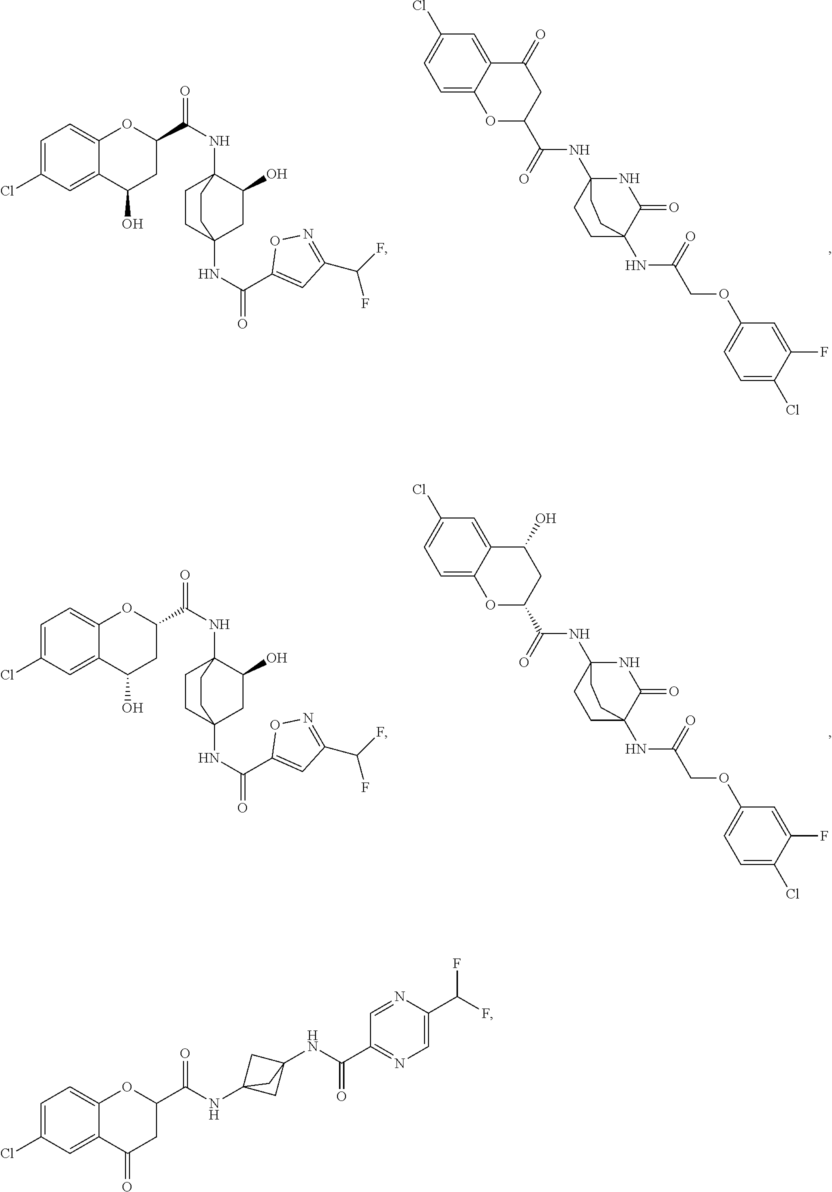 [SMILES: O=C(NC12CCC(NC(=O)[C@H]3C[C@@H](O)c4cc(Cl)ccc4O3)(CC1)[C@@H](O)C2)c1cc(C(F)F)no1 ; O=C(COc1ccc(Cl)c(F)c1)NC12CCC(NC(=O)C3CC(=O)c4cc(Cl)ccc4O3)(CC1)NC2=O ; O=C(NC12CCC(NC(=O)[C@@H]3C[C@H](O)c4cc(Cl)ccc4O3)(CC1)[C@@H](O)C2)c1cc(C(F)F)no1 ; O=C(COc1ccc(Cl)c(F)c1)NC12CCC(NC(=O)[C@H]3C[C@@H](O)c4cc(Cl)ccc4O3)(CC1)NC2=O ; O=C(NC12CC(NC(=O)C3CC(=O)c4cc(Cl)ccc4O3)(C1)C2)c1cnc(C(F)F)cn1] 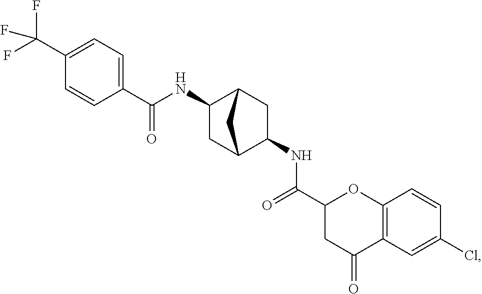 [SMILES: O=C(N[C@@H]1C[C@@H]2C[C@H]1C[C@H]2NC(=O)C1CC(=O)c2cc(Cl)ccc2O1)c1ccc(C(F)(F)F)cc1] 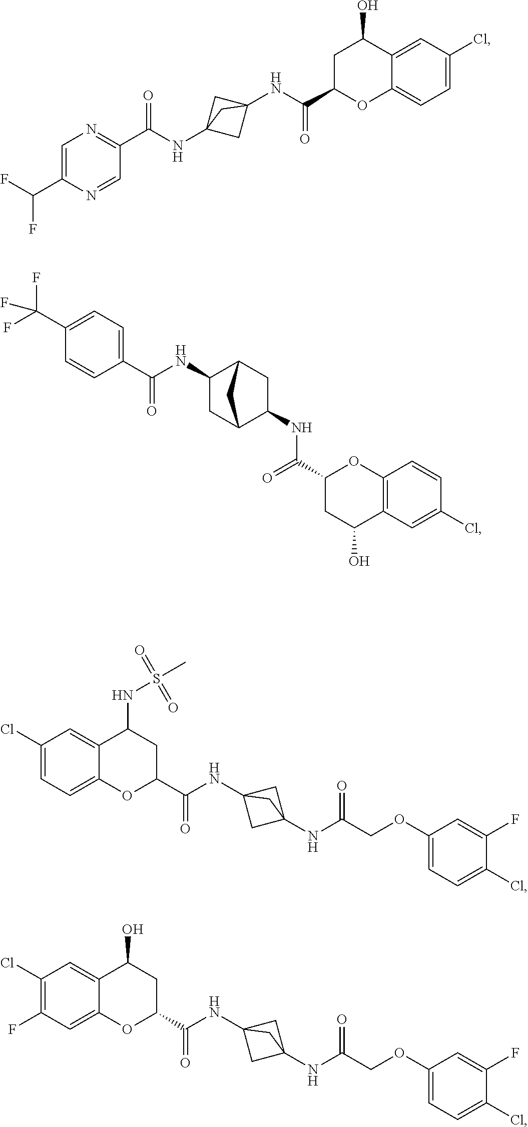 [SMILES: O=C(NC12CC(NC(=O)[C@H]3C[C@@H](O)c4cc(Cl)ccc4O3)(C1)C2)c1cnc(C(F)F)cn1 ; O=C(N[C@@H]1C[C@@H]2C[C@H]1C[C@H]2NC(=O)[C@H]1C[C@@H](O)c2cc(Cl)ccc2O1)c1ccc(C(F)(F)F)cc1 ; CS(=O)(=O)NC1CC(C(=O)NC23CC(NC(=O)COc4ccc(Cl)c(F)c4)(C2)C3)Oc2ccc(Cl)cc21 ; O=C(COc1ccc(Cl)c(F)c1)NC12CC(NC(=O)[C@H]3C[C@H](O)c4cc(Cl)c(F)cc4O3)(C1)C2] 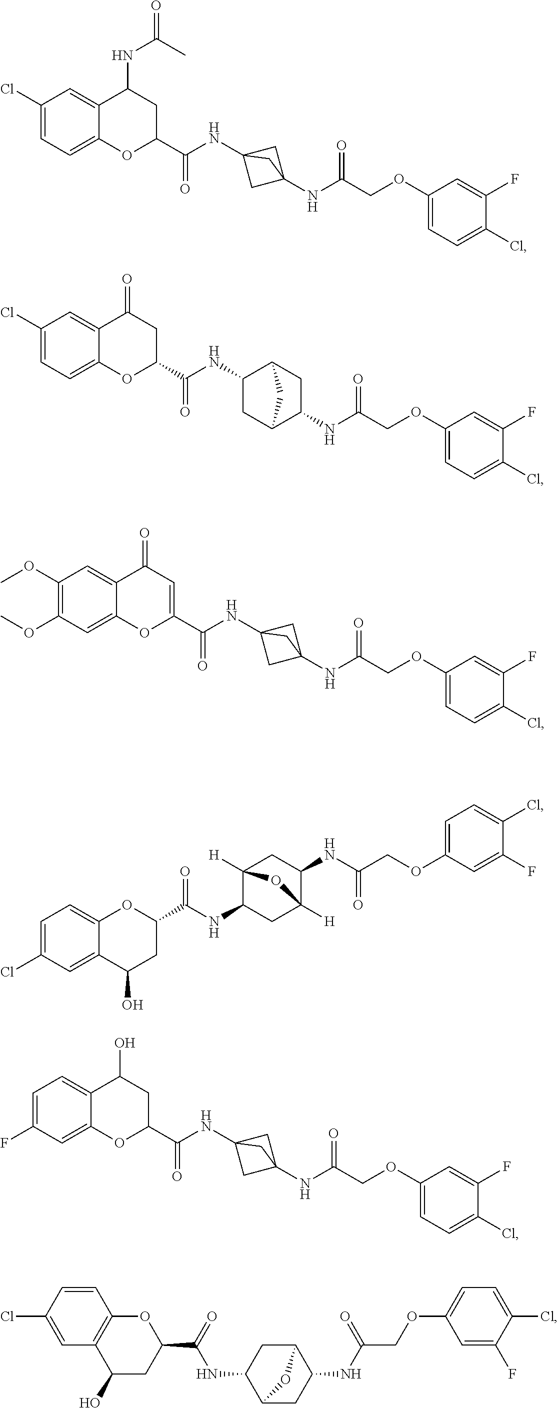 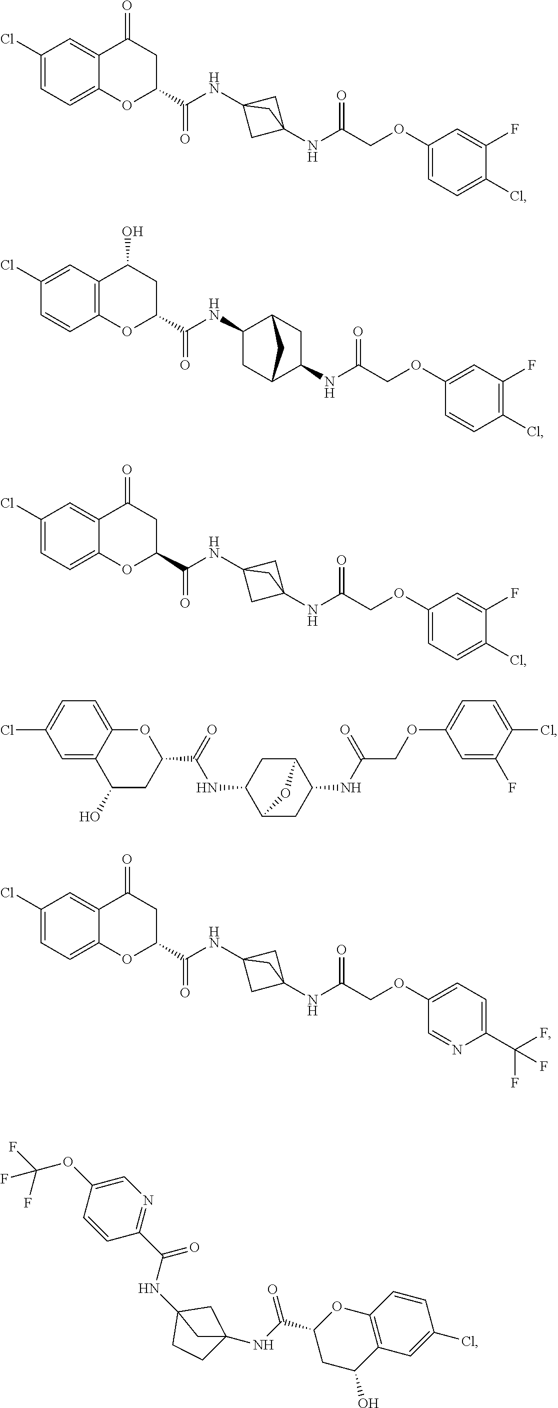 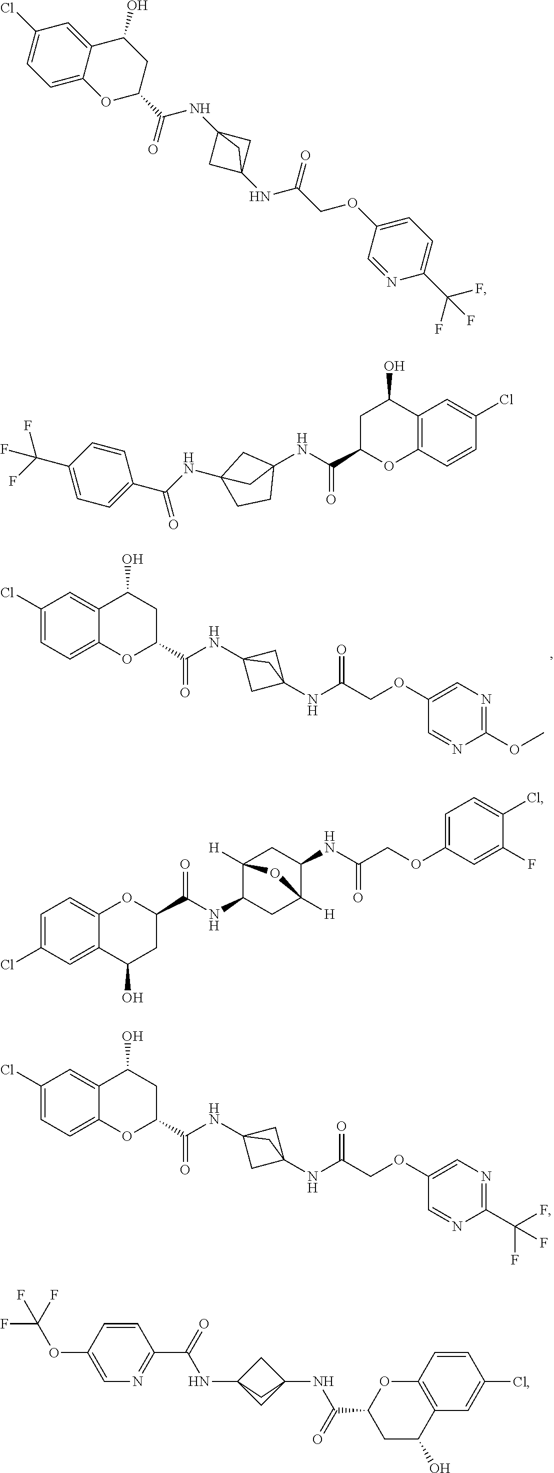 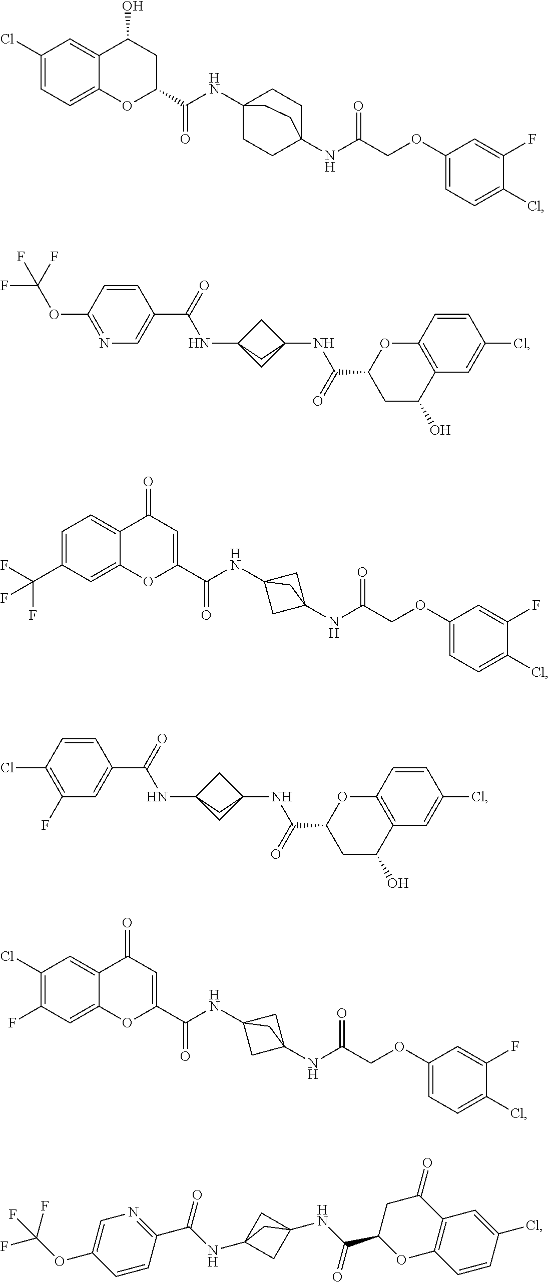 [SMILES: O=C(COc1ccc(Cl)c(F)c1)NC12CCC(NC(=O)[C@H]3C[C@@H](O)c4cc(Cl)ccc4O3)(CC1)CC2 ; O=C(NC12CC(NC(=O)[C@H]3C[C@@H](O)c4cc(Cl)ccc4O3)(C1)C2)c1ccc(OC(F)(F)F)nc1 ; O=C(COc1ccc(Cl)c(F)c1)NC12CC(NC(=O)c3cc(=O)c4ccc(C(F)(F)F)cc4o3)(C1)C2 ; O=C(NC12CC(NC(=O)[C@H]3C[C@@H](O)c4cc(Cl)ccc4O3)(C1)C2)c1ccc(Cl)c(F)c1 ; O=C(COc1ccc(Cl)c(F)c1)NC12CC(NC(=O)c3cc(=O)c4cc(Cl)c(F)cc4o3)(C1)C2 ; O=C(NC12CC(NC(=O)[C@H]3CC(=O)c4cc(Cl)ccc4O3)(C1)C2)c1ccc(OC(F)(F)F)cn1] 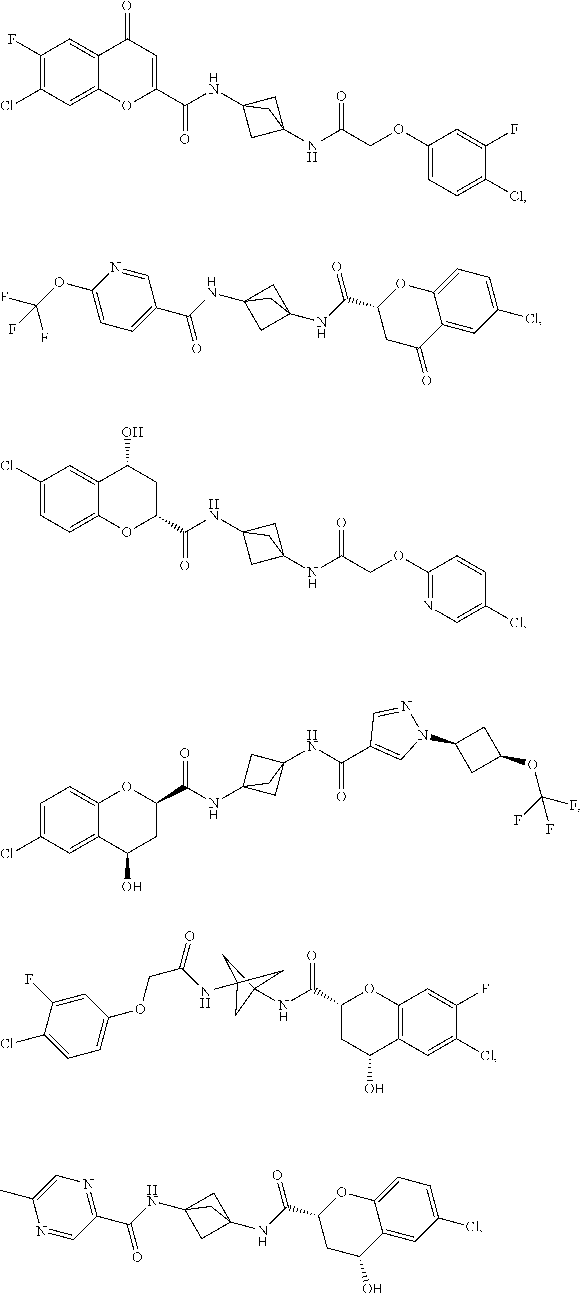 [SMILES: O=C(COc1ccc(Cl)c(F)c1)NC12CC(NC(=O)c3cc(=O)c4cc(F)c(Cl)cc4o3)(C1)C2 ; O=C(NC12CC(NC(=O)[C@H]3CC(=O)c4cc(Cl)ccc4O3)(C1)C2)c1ccc(OC(F)(F)F)nc1 ; O=C(COc1ccc(Cl)cn1)NC12CC(NC(=O)[C@H]3C[C@@H](O)c4cc(Cl)ccc4O3)(C1)C2 ; O=C(NC12CC(NC(=O)[C@H]3C[C@@H](O)c4cc(Cl)ccc4O3)(C1)C2)c1cnn([C@H]2C[C@@H](OC(F)(F)F)C2)c1 ; O=C(COc1ccc(Cl)c(F)c1)NC12CC(NC(=O)[C@H]3C[C@@H](O)c4cc(Cl)c(F)cc4O3)(C1)C2 ; Cc1cnc(C(=O)NC23CC(NC(=O)[C@H]4C[C@@H](O)c5cc(Cl)ccc5O4)(C2)C3)cn1] 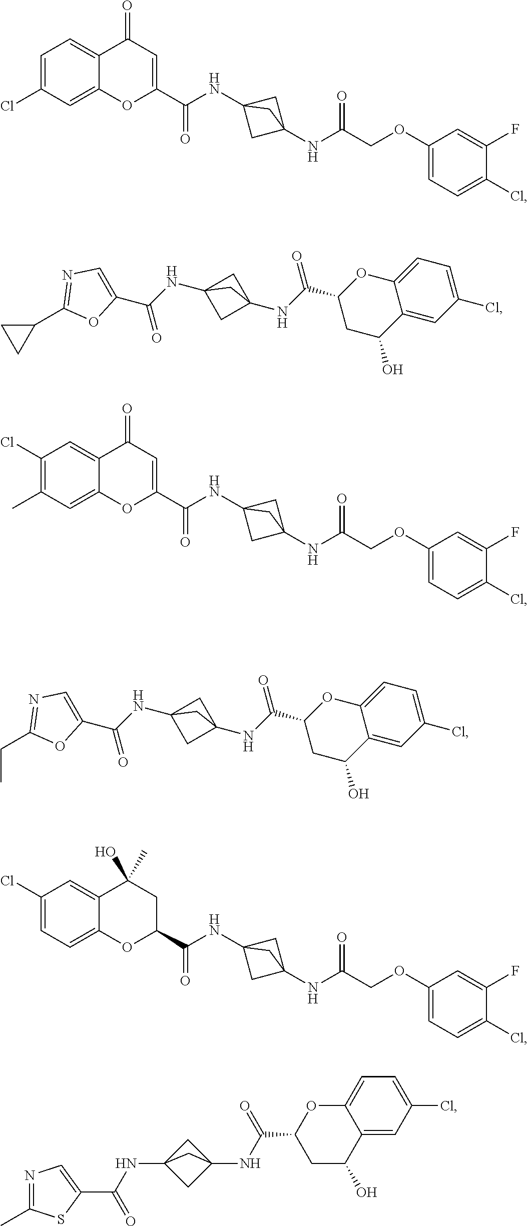 [SMILES: O=C(COc1ccc(Cl)c(F)c1)NC12CC(NC(=O)c3cc(=O)c4ccc(Cl)cc4o3)(C1)C2 ; O=C(NC12CC(NC(=O)[C@H]3C[C@@H](O)c4cc(Cl)ccc4O3)(C1)C2)c1cnc(C2CC2)o1 ; Cc1cc2oc(C(=O)NC34CC(NC(=O)COc5ccc(Cl)c(F)c5)(C3)C4)cc(=O)c2cc1Cl ; CCc1ncc(C(=O)NC23CC(NC(=O)[C@H]4C[C@@H](O)c5cc(Cl)ccc5O4)(C2)C3)o1 ; C[C@]1(O)C[C@@H](C(=O)NC23CC(NC(=O)COc4ccc(Cl)c(F)c4)(C2)C3)Oc2ccc(Cl)cc21 ; Cc1ncc(C(=O)NC23CC(NC(=O)[C@H]4C[C@@H](O)c5cc(Cl)ccc5O4)(C2)C3)s1] 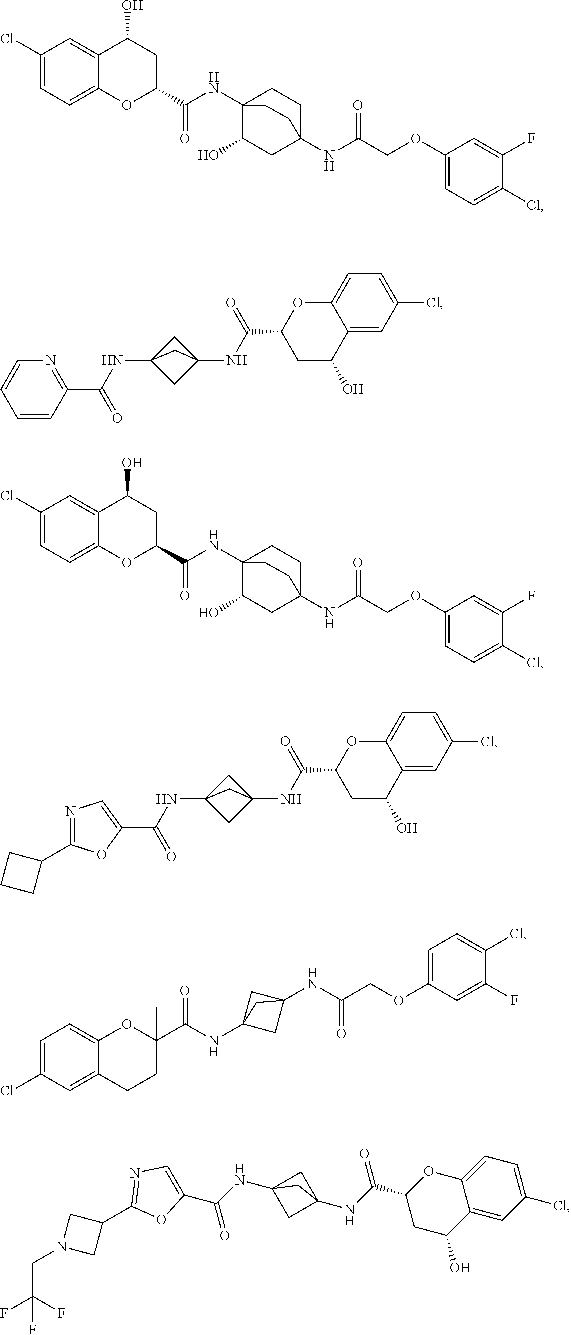 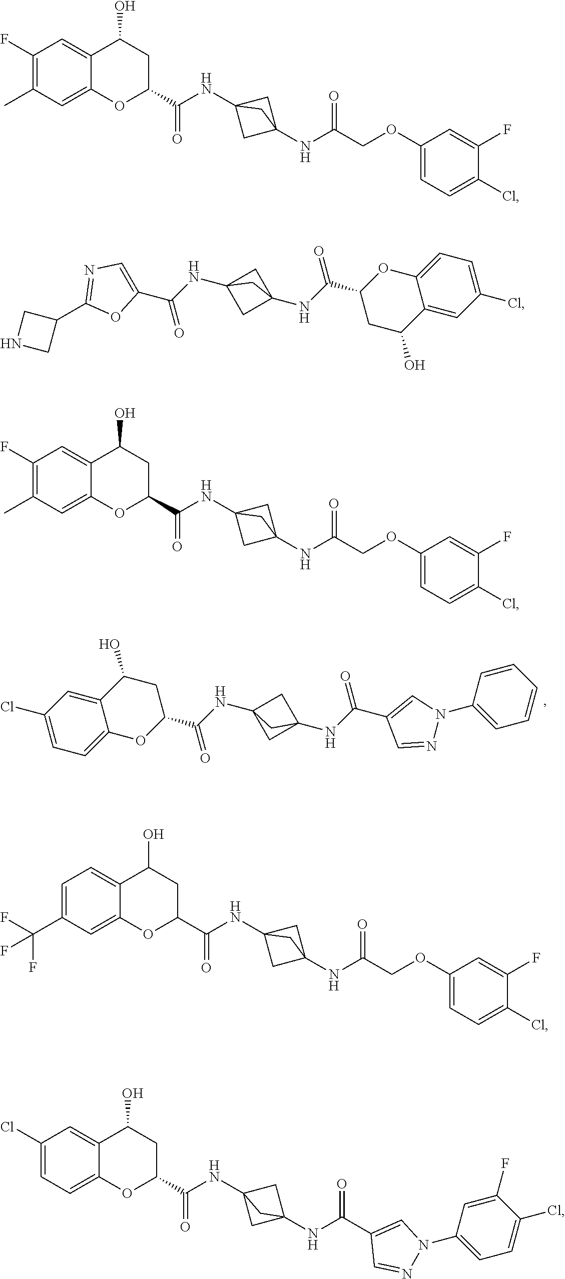 [SMILES: Cc1cc2c(cc1F)[C@H](O)C[C@H](C(=O)NC13CC(NC(=O)COc4ccc(Cl)c(F)c4)(C1)C3)O2 ; O=C(NC12CC(NC(=O)[C@H]3C[C@@H](O)c4cc(Cl)ccc4O3)(C1)C2)c1cnc(C2CNC2)o1 ; Cc1cc2c(cc1F)[C@@H](O)C[C@@H](C(=O)NC13CC(NC(=O)COc4ccc(Cl)c(F)c4)(C1)C3)O2 ; O=C(NC12CC(NC(=O)[C@H]3C[C@@H](O)c4cc(Cl)ccc4O3)(C1)C2)c1cnn(-c2ccccc2)c1 ; O=C(COc1ccc(Cl)c(F)c1)NC12CC(NC(=O)C3CC(O)c4ccc(C(F)(F)F)cc4O3)(C1)C2 ; O=C(NC12CC(NC(=O)[C@H]3C[C@@H](O)c4cc(Cl)ccc4O3)(C1)C2)c1cnn(-c2ccc(Cl)c(F)c2)c1] 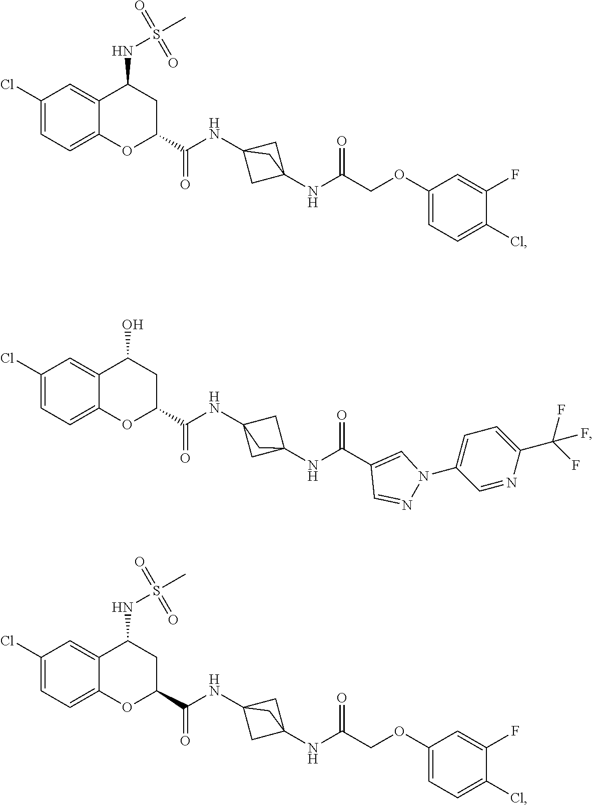 [SMILES: CS(=O)(=O)N[C@H]1C[C@H](C(=O)NC23CC(NC(=O)COc4ccc(Cl)c(F)c4)(C2)C3)Oc2ccc(Cl)cc21 ; O=C(NC12CC(NC(=O)[C@H]3C[C@@H](O)c4cc(Cl)ccc4O3)(C1)C2)c1cnn(-c2ccc(C(F)(F)F)nc2)c1 ; CS(=O)(=O)N[C@@H]1C[C@@H](C(=O)NC23CC(NC(=O)COc4ccc(Cl)c(F)c4)(C2)C3)Oc2ccc(Cl)cc21]
and a pharmaceutically acceptable salt, solvate, hydrate, tautomer, N-oxide, or stereoisomer thereof.

67. A pharmaceutically acceptable composition comprising a compound of any one of claims 1-66 and a pharmaceutically acceptable carrier.

68. A method of treating a neurodegenerative disease, a leukodystrophy, a cancer, an inflammatory disease, an autoimmune disease, a viral infection, a skin disease, a fibrotic disease, a hemoglobin disease, a kidney disease, a hearing loss condition, an ocular disease, a musculoskeletal disease, a metabolic disease, or a mitochondrial disease in a patient in need thereof, comprising administering to the patient a therapeutically effective amount of a compound of any of claims 1-66, or a pharmaceutically acceptable salt, solvate, hydrate, tautomer, N-oxide, or stereoisomer thereof.

69. The method of claim 68, wherein the neurodegenerative disease comprises a leukodystrophy, a leukoencephalopathy, a hypomyelinating or demyelinating disease, an intellectual disability syndrome, a cognitive impairment, a glial cell dysfunction, or a brain injury.

70. The method of any one of claims 68 or 69, wherein the neurodegenerative disease comprises vanishing white matter disease, childhood ataxia with CNS hypo myelination, Alzheimer's disease, amyotrophic lateral sclerosis, Creutzfeldt-Jakob disease, frontotemporal dementia, Gerstmann-Straussler-Scheinker disease, Huntington's disease, dementia, kuru, multiple sclerosis, Parkinson's disease, or a prion disease.

71. The method of any one of claims 68-70, wherein the neurodegenerative disease comprises vanishing white matter disease.

72. The method of claim 68, wherein the cancer comprises pancreatic cancer, breast cancer, multiple myeloma, or a cancer of the secretory cells.

73. The method of claim 68, wherein the inflammatory disease comprises postoperative cognitive dysfunction, arthritis, systemic lupus erythematosus (SLE), myasthenia gravis, diabetes), Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, Behcet's disease, Crohn's disease, ulcerative colitis, bullous pemphigoid, sarcoidosis, ichthyosis, Graves' ophthalmopathy, inflammatory bowel disease, Addison's disease, vitiligo, acne vulgaris, celiac disease, chronic prostatitis, pelvic inflammatory disease, reperfusion injury, sarcoidosis, transplant rejection, interstitial cystitis, atherosclerosis, or atopic dermatitis.

74. The method of claim 68, wherein the musculoskeletal disease comprises muscular dystrophy, multiple sclerosis, amyotropic lateral sclerosis, primary lateral sclerosis, progressive muscular atrophy, progressive bulbar palsy, pseudobulbar palsy, spinal muscular atrophy, progressive spinobulbar muscular atrophy, spinal cord spasticity, spinal muscle atrophy, myasthenia gravis, neuralgia, fibromyalgia, Machado-Joseph disease, cramp fasciculation syndrome, Freidrich's ataxia, a muscle wasting disorder), an inclusion body myopathy, motor neuron disease, or paralysis.

75. The composition of claim 68, wherein the metabolic disease comprises non-alcoholic steatohepatitis (NASH), non-alcoholic fatty liver disease (NAFLD), liver fibrosis, obesity, heart disease, atherosclerosis, arthritis, cystinosis, diabetes, phenylketonuria, proliferative retinopathy, or Kearns-Sayre disease.

76. The method of claim 68, wherein the mitochondrial disease is associated with or is a result of mitochondrial dysfunction, one or more mitochondrial protein mutations, or one or more mitochondrial DNA mutations.

77. The method of claim 68 or 76, wherein the mitochondrial disease is a mitochondrial myopathy.

78. The method of any one of claims 68 and 76-77, wherein the mitochondrial disease is selected from the group consisting of Barth syndrome, chronic progressive external ophthalmoplegia (cPEO), Kearns-Sayre syndrome (KSS), Leigh syndrome (e.g., MILS, or maternally inherited Leigh syndrome), mitochondrial DNA depletion syndromes (MDDS, e.g., Alpers syndrome), mitochondrial encephalomyopathy (e.g., mitochondrial encephalomyopathy, lactic acidosis, and stroke-like episodes (MELAS)), mitochondrial neurogastrointestinal encephalomyopathy (MNGIE), myoclonus epilepsy with ragged red fibers (MERRF), neuropathy, ataxia, retinitis pigmentosa (NARP), Leber's hereditary optic neuropathy (LHON) and Pearson syndrome.

79. The method of claim 68, wherein the autoimmune disease is selected from the group consisting of Achalasia, Addison's disease, Adult Still's disease, Agammaglobulinemia, Alopecia areata, Amyloidosis, Ankylosing spondylitis, Anti-GBM/Anti-TBM nephritis, Antiphospholipid syndrome, Autoimmune angioedema, Autoimmune dysautonomia, Autoimmune encephalomyelitis, Autoimmune hepatitis, Autoimmune inner ear disease (AIED), Autoimmune myocarditis, Autoimmune oophoritis, Autoimmune orchitis, Autoimmune pancreatitis, Autoimmune retinopathy, Autoimmune urticaria, Axonal & neuronal neuropathy (AMAN), Baló disease, Behcet's disease, Benign mucosal pemphigoid, Bullous pemphigoid, Castleman disease (CD), Celiac disease, Chagas disease, Chronic inflammatory demyelinating polyneuropathy (CIDP), Chronic recurrent multifocal osteomyelitis (CRMO), Churg-Strauss Syndrome (CSS) or Eosinophilic Granulomatosis (EGPA), Cicatricial pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease (neuromyelitis optica), Discoid lupus, Dressler's syndrome, Endometriosis, Eosinophilic esophagitis (EoE), Eosinophilic fasciitis, Erythema nodosum, Essential mixed cryoglobulinemia, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's thyroiditis, Hemolytic anemia, Henoch-Schonlein purpura (HSP), Herpes gestationis or pemphigoid gestationis (PG), Hidradenitis Suppurativa (HS) (Acne Inversa), Hypogammalglobulinemia, IgA Nephropathy, IgG4-related sclerosing disease, Immune thrombocytopenic purpura (ITP), Inclusion body myositis (IBM), Interstitial cystitis (IC), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus, Lyme disease chronic, Meniere's disease, Microscopic polyangiitis (MPA), Mixed connective tissue disease (MCTD), Mooren's ulcer, Mucha-Habermann disease, Multifocal Motor Neuropathy (MMN) or MMNCB, Multiple sclerosis, Myasthenia gravis, Myositis, Narcolepsy, Neonatal Lupus, Neuromyelitis optica, Neutropenia, Ocular cicatricial pemphigoid, Optic neuritis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonnage-Turner syndrome, Pemphigus, Peripheral neuropathy, Perivenous encephalomyelitis, Pernicious anemia (PA), POEMS syndrome, Polyarteritis nodosa, Polyglandular syndrome type I, Polyglandular syndrome type II, Polyglandular syndrome type III, Polymyalgia rheumatica, Polymyositis, Postmyocardial infarction syndrome, Postpericardiotomy syndrome, Primary biliary cirrhosis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Sarcoidosis, Schmidt syndrome, Scleritis, Scleroderma, Sjogren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopenic purpura (TTP), Tolosa-Hunt syndrome (THS), Transverse myelitis, Type 1 diabetes, Ulcerative colitis (UC), Undifferentiated connective tissue disease (UCTD), Uveitis, Vasculitis, Vitiligo, Vogt-Koyanagi-Harada Disease, and Wegener's granulomatosis (or Granulomatosis with Polyangiitis (GPA)).

80. The method of claim 68, wherein the viral infection is selected from the group consisting of influenza, human immunodeficiency virus (HIV) and herpes.

81. The method of claim 68, wherein the skin disease is selected from the group consisting of acne, alopecia areata, basal cell carcinoma, Bowen's disease, congenital erythropoietic porphyria, contact dermatitis, Darier's disease, disseminated superficial actinic porokeratosis, dystrophic epidermolysis bullosa, eczema (atopic eczema), extra-mammary Paget's disease, epidermolysis bullosa simplex, erythropoietic protoporphyria, fungal infections of nails, Hailey-Hailey disease, herpes simplex, hidradenitis suppurativa, hirsutism, hyperhidrosis, ichthyosis, impetigo, keloids, keratosis pilaris, lichen planus, lichen sclerosus, melanoma, melasma, mucous membrane pemphigoid, pemphigoid, Pemphigus vulgaris, Pityriasis lichenoides, Pityriasis rubra pilaris, plantar warts (verrucas), polymorphic light eruption, psoriasis, plaque psoriasis, pyoderma gangrenosum, rosacea, scabies, scleroderma, shingles, squamous cell carcinoma, sweet's syndrome, urticaria and angioedema and vitiligo.

82. The method of claim 68, wherein the fibrotic disease is selected from the group consisting of adhesive capsulitis, arterial stiffness, arthrofibrosis, atrial fibrosis, cardiac fibrosis, cirrhosis, congenital hepatic fibrosis, Crohn's disease, cystic fibrosis, Dupuytren's contracture, endomyocardial fibrosis, glial scar, hepatitis C, hypertrophic cardiomyopathy, hypersensitivity pneumonitis, idiopathic pulmonary fibrosis, idiopathic interstitial pneumonia, interstitial lung disease, keloid, mediastinal fibrosis, myelofibrosis, nephrogenic systemic fibrosis, non-alcoholic fatty liver disease, old myocardial infarction, Peyronie's disease, pneumoconiosis, pneumonitis, progressive massive fibrosis, pulmonary fibrosis, radiation-induced lung injury, retroperitoneal fibrosis, scleroderma/systemic sclerosis, silicosis and ventricular remodeling.

83. The method of claim 68, wherein the hemoglobin disease is selected from the group consisting of “dominant” β-thalassemia, acquired (toxic) methemoglobinemia, carboxyhemoglobinemia, congenital Heinz body hemolytic anemia, HbH disease, HbS/β-thalassemia, HbE/β-thalassemia, HbSC disease, homozygous α⁺-thalassemia (phenotype of α⁰-thalassemia), Hydrops fetalis with Hb Bart's, sickle cell anemia/disease, sickle cell trait, sickle β-thalassemia disease, α⁺-thalassemia, α⁰-thalassemia, α-Thalassemia associated with myelodysplastic syndromes, α-Thalassemia with mental retardation syndrome (ATR), β⁰-Thalassemia, β⁺-Thalassemia, δ-Thalassemia, γ-Thalassemia, β-Thalassemia major, β-Thalassemia intermedia, δβ-Thalassemia, and εγδβ-Thalassemia.

84. The method of claim 68, wherein the kidney disease is selected from the group consisting of Abderhalden-Kaufmann-Lignac syndrome (Nephropathic Cystinosis), Abdominal Compartment Syndrome, Acetaminophen-induced Nephrotoxicity, Acute Kidney Failure/Acute Kidney Injury, Acute Lobar Nephronia, Acute Phosphate Nephropathy, Acute Tubular Necrosis, Adenine Phosphoribosyltransferase Deficiency, Adenovirus Nephritis, Alagille Syndrome, Alport Syndrome, Amyloidosis, ANCA Vasculitis Related to Endocarditis and Other Infections, Angiomyolipoma, Analgesic Nephropathy, Anorexia Nervosa and Kidney Disease, Angiotensin Antibodies and Focal Segmental Glomerulosclerosis, Antiphospholipid Syndrome, Anti-TNF-α Therapy-related Glomerulonephritis, APOL1 Mutations, Apparent Mineralocorticoid Excess Syndrome, Aristolochic Acid Nephropathy, Chinese Herbal Nephropathy, Balkan Endemic Nephropathy, Arteriovenous Malformations and Fistulas of the Urologic Tract, Autosomal Dominant Hypocalcemia, Bardet-Biedl Syndrome, Bartter Syndrome, Bath Salts and Acute Kidney Injury, Beer Potomania, Beeturia, β-Thalassemia Renal Disease, Bile Cast Nephropathy, BK Polyoma Virus Nephropathy in the Native Kidney, Bladder Rupture, Bladder Sphincter Dyssynergia, Bladder Tamponade, Border-Crossers' Nephropathy, Bourbon Virus and Acute Kidney Injury, Burnt Sugarcane Harvesting and Acute Renal Dysfunction, Byetta and Renal Failure, C1q Nephropathy, C3 Glomerulopathy, C3 Glomerulopathy with Monoclonal Gammopathy, C4 Glomerulopathy, Calcineurin Inhibitor Nephrotoxicity, Callilepsis Laureola Poisoning, Cannabinoid Hyperemesis Acute Renal Failure, Cardiorenal syndrome, Carfilzomib-Induced Renal Injury, CFHR5 nephropathy, Charcot-Marie-Tooth Disease with Glomerulopathy, Chinese Herbal Medicines and Nephrotoxicity, Cherry Concentrate and Acute Kidney Injury, Cholesterol Emboli, Churg-Strauss syndrome, Chyluria, Ciliopathy, Cocaine and the Kidney, Cold Diuresis, Colistin Nephrotoxicity, Collagenofibrotic Glomerulopathy, Collapsing Glomerulopathy, Collapsing Glomerulopathy Related to CMV, Combination Antiretroviral (cART) Related-Nephropathy, Congenital Anomalies of the Kidney and Urinary Tract (CAKUT), Congenital Nephrotic Syndrome, Congestive Renal Failure, Conorenal syndrome (Mainzer-Saldino Syndrome or Saldino-Mainzer Disease), Contrast Nephropathy, Copper Sulphate Intoxication, Cortical Necrosis, Crizotinib-related Acute Kidney Injury, Cryocrystalglobulinemia, Cryoglobuinemia, Crystalglobulin-Induced Nephropathy, Crystal-Induced Acute Kidney injury, Crystal-Storing Histiocytosis, Cystic Kidney Disease, Acquired, Cystinuria, Dasatinib-Induced Nephrotic-Range Proteinuria, Dense Deposit Disease (MPGN Type 2), Dent Disease (X-linked Recessive Nephrolithiasis), DHA Crystalline Nephropathy, Dialysis Disequilibrium Syndrome, Diabetes and Diabetic Kidney Disease, Diabetes Insipidus, Dietary Supplements and Renal Failure, Diffuse Mesangial Sclerosis, Diuresis, Djenkol Bean Poisoning (Djenkolism), Down Syndrome and Kidney Disease, Drugs of Abuse and Kidney Disease, Duplicated Ureter, EAST syndrome, Ebola and the Kidney, Ectopic Kidney, Ectopic Ureter, Edema, Swelling, Erdheim-Chester Disease, Fabry's Disease, Familial Hypocalciuric Hypercalcemia, Fanconi Syndrome, Fraser syndrome, Fibronectin Glomerulopathy, Fibrillary Glomerulonephritis and Immunotactoid Glomerulopathy, Fraley syndrome, Fluid Overload, Hypervolemia, Focal Segmental Glomerulosclerosis, Focal Sclerosis, Focal Glomerulosclerosis, Galloway Mowat syndrome, Giant Cell (Temporal) Arteritis with Kidney Involvement, Gestational Hypertension, Gitelman Syndrome, Glomerular Diseases, Glomerular Tubular Reflux, Glycosuria, Goodpasture Syndrome, Green Smoothie Cleanse Nephropathy, HANAC Syndrome, Harvoni (Ledipasvir with Sofosbuvir)-Induced Renal Injury, Hair Dye Ingestion and Acute Kidney Injury, Hantavirus Infection Podocytopathy, Heat Stress Nephropathy, Hematuria (Blood in Urine), Hemolytic Uremic Syndrome (HUS), Atypical Hemolytic Uremic Syndrome (aHUS), Hemophagocytic Syndrome, Hemorrhagic Cystitis, Hemorrhagic Fever with Renal Syndrome (HFRS, Hantavirus Renal Disease, Korean Hemorrhagic Fever, Epidemic Hemorrhagic Fever, Nephropathis Epidemica), Hemosiderinuria, Hemosiderosis related to Paroxysmal Nocturnal Hemoglobinuria and Hemolytic Anemia, Hepatic Glomerulopathy, Hepatic Veno-Occlusive Disease, Sinusoidal Obstruction Syndrome, Hepatitis C-Associated Renal Disease, Hepatocyte Nuclear Factor 1β-Associated Kidney Disease, Hepatorenal Syndrome, Herbal Supplements and Kidney Disease, High Altitude Renal Syndrome, High Blood Pressure and Kidney Disease, HIV-Associated Immune Complex Kidney Disease (HIVICK), HIV-Associated Nephropathy (HIVAN), HNF1B-related Autosomal Dominant Tubulointerstitial Kidney Disease, Horseshoe Kidney (Renal Fusion), Hunner's Ulcer, Hydroxychloroquine-induced Renal Phospholipidosis, Hyperaldosteronism, Hypercalcemia, Hyperkalemia, Hypermagnesemia, Hypernatremia, Hyperoxaluria, Hyperphosphatemia, Hypocalcemia, Hypocomplementemic Urticarial Vasculitic Syndrome, Hypokalemia, Hypokalemia-induced renal dysfunction, Hypokalemic Periodic Paralysis, Hypomagnesemia, Hyponatremia, Hypophosphatemia, Hypophosphatemia in Users of Cannabis, Hypertension, Hypertension, Monogenic, Iced Tea Nephropathy, Ifosfamide Nephrotoxicity, IgA Nephropathy, IgG4 Nephropathy, Immersion Diuresis, Immune-Checkpoint Therapy-Related Interstitial Nephritis, Infliximab-Related Renal Disease, Interstitial Cystitis, Painful Bladder Syndrome (Questionnaire), Interstitial Nephritis, Interstitial Nephritis, Karyomegalic, Ivemark's syndrome, JC Virus Nephropathy, Joubert Syndrome, Ketamine-Associated Bladder Dysfunction, Kidney Stones, Nephrolithiasis, Kombucha Tea Toxicity, Lead Nephropathy and Lead-Related Nephrotoxicity, Lecithin Cholesterol Acyltransferase Deficiency (LCAT Deficiency), Leptospirosis Renal Disease, Light Chain Deposition Disease, Monoclonal Immunoglobulin Deposition Disease, Light Chain Proximal Tubulopathy, Liddle Syndrome, Lightwood-Albright Syndrome, Lipoprotein Glomerulopathy, Lithium Nephrotoxicity, LMX1B Mutations Cause Hereditary FSGS, Loin Pain Hematuria, Lupus, Systemic Lupus Erythematosis, Lupus Kidney Disease, Lupus Nephritis, Lupus Nephritis with Antineutrophil Cytoplasmic Antibody Seropositivity, Lupus Podocytopathy, Lyme Disease-Associated Glomerulonephritis, Lysinuric Protein Intolerance, Lysozyme Nephropathy, Malarial Nephropathy, Malignancy-Associated Renal Disease, Malignant Hypertension, Malakoplakia, McKittrick-Wheelock Syndrome, MDMA (Molly; Ecstacy; 3,4-Methylenedioxymethamphetamine) and Kidney Failure, Meatal Stenosis, Medullary Cystic Kidney Disease, Urolodulin-Associated Nephropathy, Juvenile Hyperuricemic Nephropathy Type 1, Medullary Sponge Kidney, Megaureter, Melamine Toxicity and the Kidney, MELAS Syndrome, Membranoproliferative Glomerulonephritis, Membranous Nephropathy, Membranous-like Glomerulopathy with Masked IgG Kappa Deposits, MesoAmerican Nephropathy, Metabolic Acidosis, Metabolic Alkalosis, Methotrexate-related Renal Failure, Microscopic Polyangiitis, Milk-alkalai syndrome, Minimal Change Disease, Monoclonal Gammopathy of Renal Significance, Dysproteinemia, Mouthwash Toxicity, MUC1 Nephropathy, Multicystic dysplastic kidney, Multiple Myeloma, Myeloproliferative Neoplasms and Glomerulopathy, Nail-patella Syndrome, NARP Syndrome, Nephrocalcinosis, Nephrogenic Systemic Fibrosis, Nephroptosis (Floating Kidney, Renal Ptosis), Nephrotic Syndrome, Neurogenic Bladder, 9/11 and Kidney Disease, Nodular Glomerulosclerosis, Non-Gonococcal Urethritis, Nutcracker syndrome, Oligomeganephronia, Orofaciodigital Syndrome, Orotic Aciduria, Orthostatic Hypotension, Orthostatic Proteinuria, Osmotic Diuresis, Osmotic Nephrosis, Ovarian Hyperstimulation Syndrome, Oxalate Nephropathy, Page Kidney, Papillary Necrosis, Papillorenal Syndrome (Renal-Coloboma Syndrome, Isolated Renal Hypoplasia), PARN Mutations and Kidney Disease, Parvovirus B19 and the Kidney, The Peritoneal-Renal Syndrome, POEMS Syndrome, Posterior Urethral Valve, Podocyte Infolding Glomerulopathy, Post-infectious Glomerulonephritis, Post-streptococcal Glomerulonephritis, Post-infectious Glomerulonephritis, Atypical, Post-Infectious Glomerulonephritis (IgA-Dominant), Mimicking IgA Nephropathy, Polyarteritis Nodosa, Polycystic Kidney Disease, Posterior Urethral Valves, Post-Obstructive Diuresis, Preeclampsia, Propofol infusion syndrome, Proliferative Glomerulonephritis with Monoclonal IgG Deposits (Nasr Disease), Propolis (Honeybee Resin) Related Renal Failure, Proteinuria (Protein in Urine), Pseudohyperaldosteronism, Pseudohypobicarbonatemia, Pseudohypoparathyroidism, Pulmonary-Renal Syndrome, Pyelonephritis (Kidney Infection), Pyonephrosis, Pyridium and Kidney Failure, Radiation Nephropathy, Ranolazine and the Kidney, Refeeding syndrome, Reflux Nephropathy, Rapidly Progressive Glomerulonephritis, Renal Abscess, Peripnephric Abscess, Renal Agenesis, Renal Arcuate Vein Microthrombi-Associated Acute Kidney Injury, Renal Artery Aneurysm, Renal Artery Dissection, Spontaneous, Renal Artery Stenosis, Renal Cell Cancer, Renal Cyst, Renal Hypouricemia with Exercise-induced Acute Renal Failure, Renal Infarction, Renal Osteodystrophy, Renal Tubular Acidosis, Renin Mutations and Autosomal Dominant Tubulointerstitial Kidney Disease, Renin Secreting Tumors (Juxtaglomerular Cell Tumor), Reset Osmostat, Retrocaval Ureter, Retroperitoneal Fibrosis, Rhabdomyolysis, Rhabdomyolysis related to Bariatric Sugery, Rheumatoid Arthritis-Associated Renal Disease, Sarcoidosis Renal Disease, Salt Wasting, Renal and Cerebral, Schistosomiasis and Glomerular Disease, Schimke immuno-osseous dysplasia, Scleroderma Renal Crisis, Serpentine Fibula-Polycystic Kidney Syndrome, Exner Syndrome, Sickle Cell Nephropathy, Silica Exposure and Chronic Kidney Disease, Sri Lankan Farmers' Kidney Disease, Sjogren's Syndrome and Renal Disease, Synthetic Cannabinoid Use and Acute Kidney Injury, Kidney Disease Following Hematopoietic Cell Transplantation, Kidney Disease Related to Stem Cell Transplantation, TAFRO Syndrome, Tea and Toast Hyponatremia, Tenofovir-Induced Nephrotoxicity, Thin Basement Membrane Disease, Benign Familial Hematuria, Thrombotic Microangiopathy Associated with Monoclonal Gammopathy, Trench Nephritis, Trigonitis, Tuberculosis, Genitourinary, Tuberous Sclerosis, Tubular Dysgenesis, Immune Complex Tubulointerstitial Nephritis Due to Autoantibodies to the Proximal Tubule Brush Border, Tumor Lysis Syndrome, Uremia, Uremic Optic Neuropathy, Ureteritis Cystica, Ureterocele, Urethral Caruncle, Urethral Stricture, Urinary Incontinence, Urinary Tract Infection, Urinary Tract Obstruction, Urogenital Fistula, Uromodulin-Associated Kidney Disease, Vancomycin-Associated Cast Nephropathy, Vasomotor Nephropathy, Vesicointestinal Fistula, Vesicoureteral Reflux, VGEF Inhibition and Renal Thrombotic Microangiopathy, Volatile Anesthetics and Acute Kidney Injury, Von Hippel-Lindau Disease, Waldenstrom's Macroglobulinemic Glomerulonephritis, Warfarin-Related Nephropathy, Wasp Stings and Acute Kidney Injury, Wegener's Granulomatosis, Granulomatosis with Polyangiitis, West Nile Virus and Chronic Kidney Disease, Wunderlich syndrome, Zellweger Syndrome, or Cerebrohepatorenal Syndrome.

85. The method of claim 68, wherein the hearing loss condition is selected from the group consisting of mitochondrial nonsyndromic hearing loss and deafness, hair cell death, age-related hearing loss, noise-induced hearing loss, genetic or inherited hearing loss, hearing loss experienced as a result of ototoxic exposure, hearing loss resulting from disease, and hearing loss resulting from trauma.

86. The method of claim 68, wherein the ocular disease cataracts, glaucoma, endoplasmic reticulum (ER) stress, autophagy deficiency, age-related macular degeneration (AMD), or diabetic retinopathy.

87. The method of any one of claims 68-86, further comprising a second agent for treating a neurodegenerative disease, a leukodystrophy, a cancer, an inflammatory disease, an autoimmune disease, a viral infection, a skin disease, a fibrotic disease, a hemoglobin disease, a kidney disease, a hearing loss condition, an ocular disease, a musculoskeletal disease, a metabolic disease, a mitochondrial disease, or a disease or disorder associated with impaired function of eIF2B, eIF2α, or a component of the eIF2 pathway or ISR pathway.

88. A method of treating a disease related to a modulation of eIF2B activity or levels, eIF2α activity or levels, or the activity or levels of a component of the eIF2 pathway or the ISR pathway in a patient in need thereof, comprising administering to the patient a therapeutically effective amount of a compound of any one of claims 1-66, or a pharmaceutically acceptable salt, solvate, hydrate, tautomer, N-oxide, or stereoisomer thereof.

89. The method of claim 88, wherein the modulation comprises an increase in eIF2B activity or levels, increase in eIF2α activity or levels, or increase in activity or levels of a component of the eIF2 pathway or the ISR pathway.

90. The method of claim 88, wherein the disease may be caused by a mutation to a gene or protein sequence related to a member of the eIF2 pathway.

91. A method of treating cancer in a subject in need thereof, comprising administering to the subject a compound of any one of claims 1-66 in combination with an immunotherapeutic agent.
